SEQUENCE LISTING (2 sequences)

SEQ ID NO: 1 - DNA, 2841 bp, synthetic construct
  source 1..2841: mol_type = other DNA
atgcatcaccatcaccatcaccatcacgagaacctgtacttccagggatccatggaaccccgtgatggtagccccgaagctcgtagctccgattccgagtccgccagcgcttcctcctccggtagcgaacgtgacgctggtcccgagcccgacaaagctccccgtcgtctgaataagcgccgttttcccggtctccgtctgttcggccaccgcaaggccatcactaagtccggtctccagcatctggctcctcctcctcctacccccggtgctccttgctccgaatccgagcgccagattcgctccactgtggattggtccgaaagcgccacctatggtgagcatatctggttcgagaccaacgtctccggcgacttctgttatgtcggtgagcaatactgtgtggctcgtatgctgcagaagtccgtgtcccgccgtaaatgcgccgcttgcaaaatcgtggtccataccccttgcatcgagcaactggagaaaatcaacttccgctgcaagcccagctttcgtgagtccggttcccgcaacgtgcgcgaacctactttcgtgcgccaccactgggtgcatcgtcgtcgccaagacggcaaatgccgccactgcggcaaaggttttcagcagaaattcaccttccacagcaaggagatcgtcgccatcagctgcagctggtgcaaacaagcttaccattccaaagtgagctgcttcatgctccagcagatcgaagagccttgctctctgggtgtgcatgctgctgtcgtgattccccctacttggattctgcgtgctcgccgtccccagaacactctgaaggcctccaaaaagaagaagcgcgccagcttcaagcgtaagagctccaaaaagggtcccgaagagggccgttggcgtcccttcatcatccgccctactccttcccctctgatgaagcctctgctggtcttcgtcaaccctaagagcggcggcaaccaaggtgctaaaatcatccagtccttcctctggtatctgaaccctcgtcaagtgttcgacctcagccaaggcggtcctaaggaggctctggagatgtaccgcaaggtccacaatctgcgcatcctcgcttgtggtggcgatggcaccgtgggctggattctgtccactctggaccaactgcgtctgaaacctcccccccccgtggctattctgcctctcggtaccggcaacgatctggctcgtactctgaattggggtggtggctacaccgatgagcccgtgtccaagattctgtcccacgtcgaagaaggcaatgtcgtccaactggaccgttgggacctccacgccgaacccaaccccgaggctggccccgaggaccgtgacgagggcgctactgaccgtctgcccctcgacgtcttcaataattacttctctctgggctttgacgctcacgtgactctggaatttcatgaaagccgcgaggccaaccccgagaagttcaattcccgtttccgcaacaagatgttctacgctggcaccgccttcagcgacttcctcatgggctccagcaaggacctcgctaagcatatccgcgtggtgtgcgatggcatggatctgacccctaagatccaagatctgaagccccaatgtgtcgtgtttctgaacatcccccgctactgcgctggtactatgccttggggccatcccggtgaacaccatgacttcgaacctcagcgtcatgatgacggctatctggaggtgatcggtttcaccatgacctccctcgctgctctgcaagtgggtggccacggcgaacgtctgactcaatgccgcgaggtggtgctgaccaccagcaaagccatccccgtccaagtggatggtgagccttgcaagctggccgcctcccgtatccgtatcgctctccgcaatcaagctaccatggtccagaaggccaaacgccgcagcgctgctcctctccacagcgaccaacaacccgtccccgaacagctgcgcatccaagtgtcccgtgtcagcatgcatgactacgaggctctgcactacgacaaggaacagctgaaggaagccagcgtgcctctgggtactgtggtcgtgcccggtgacagcgatctggagctctgccgtgcccacatcgagcgtctgcagcaagagcccgacggtgctggtgccaagagccctacttgccaaaaactctcccccaagtggtgtttcctcgacgctaccaccgccagccgcttctaccgcattgatcgcgcccaagagcatctgaactatgtcaccgagatcgctcaagacgagatctacatcctcgaccccgaactcctcggtgctagcgcccgtcccgacctccccactcctacctcccctctgcccacttccccttgttcccccacccctcgtagcctccaaggtgatgctgcccctcctcaaggtgaggagctcattgaggccgctaagcgtaacgatttctgcaagctccaagagctgcatcgtgctggtggcgacctcatgcaccgcgatgagcagagccgcactctgctgcaccacgctgtgtccactggtagcaaggacgtggtgcgctatctgctggaccacgctcctcccgagatcctcgacgctgtggaagaaaacggcgagacttgcctccaccaagctgctgctctgggtcaacgtaccatctgccactacatcgtcgaagctggtgcttctctgatgaagaccgaccagcaaggtgatactccccgtcagcgcgccgagaaagcccaagacaccgaactggctgcctatctggagaaccgtcagcactaccagatgattcagcgtgaagaccaagagaccgccgtgtaa

SEQ ID NO: 2 - protein, 929 aa, Homo sapiens
MEPRDGSPEARSSDSESASASSSGSERDAGPEPDKAPRRLNKRRFPGLRLFGHRKAITKSGLQHLAPPPPTPGAPCSESERQIRSTVDWSESATYGEHIWFETNVSGDFCYVGEQYCVARMLQKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAV

(19) United States
(12) Patent Application Publication
(10) Pub. No.: US 2025/0353846 A1
(43) Pub. Date: Nov. 20, 2025

(54) Heterocyclic Compounds

(71) Applicant: BeOne Medicines I GmbH, Basel (CH)

(72) Inventors: Guoliang Zhang, Beijing (CN); Zhikun Ni, Beijing (CN); Jianzhuang Miao, Beijing (CN); Zhi Zhang, Beijing (CN); Ce Wang, Beijing (CN)

(73) Assignee: BeOne Medicines I GmbH, Basel (CH)

(21) Appl. No.: 19/283,966

(22) Filed: Jul. 29, 2025

Related U.S. Application Data
(63) Continuation of application No. PCT/CN2024/075494, filed on Feb. 2, 2024.

(30) Foreign Application Priority Data
Feb. 2, 2023 (WO) PCT/CN2023/074235

(51) Int. Cl. C07D 471/04; A61K 31/4188; A61K 31/426; A61K 31/4355; A61K 31/436; A61K 31/522; C07D 473/28; C07D 491/048; C07D 491/052; C07D 491/056; C07D 513/04

(52) U.S. Cl. CPC C07D 471/04; A61K 31/4188; A61K 31/426; A61K 31/4355; A61K 31/436; A61K 31/522; C07D 473/28; C07D 491/048; C07D 491/052; C07D 491/056; C07D 513/04

(57) ABSTRACT

Disclosed herein is a compound of Formula (I) having the following structure for activating T cells, promoting T cell proliferation, and/or exhibiting antitumor activity, a method of using the compounds disclosed herein for treating cancer, and a pharmaceutical composition comprising the same.

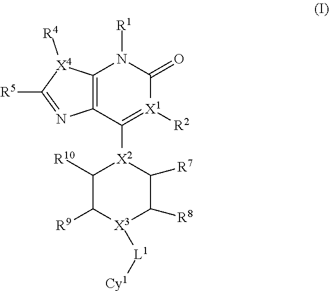

### CROSS-REFERENCE TO RELATED APPLICATIONS

This application is a continuation of International Application No. PCT/CN2024/075494, filed on Feb. 2, 2024, which claims priority to International Application No. PCT/CN2023/074235, filed on Feb. 2, 2023, the disclosures of each of which are hereby incorporated by reference in their entireties.

### SEQUENCE LISTING

This application contains a Sequence Listing, which has been submitted electronically in XML format. The XML file entitled “2024-05-15_01368-0102-00PCT_Sequence listing as filed,” was created on May 15, 2025, and is 6,531 bytes in size. The Sequence Listing is incorporated herein by reference in its entirety.

### FIELD OF THE DISCLOSURE

Disclosed herein is a compound of Formula (I) for activating T cells, promoting T cell proliferation, and/or exhibiting antitumor activity, a method of using the compounds disclosed herein for treating cancer, and a pharmaceutical composition comprising the same.

### BACKGROUND OF THE DISCLOSURE

Diacylglycerol kinases (DGKs) are a family of lipid kinases that phosphorylates and converts diacylglycerol (DAG) into phosphatidic acid (PA). As the substrate of DGKs, DAG is generated from inositol phospholipids and other phospholipids at the plasma membrane by phospholipase C (PLC) hydrolysis in response to the activation of various cell-surface receptors, including G-protein coupled receptors (GPCR) and immunoreceptor tyrosine-based activation motif (ITAM)-bearing receptors (Rhee, Sue Goo. Annual review of biochemistry. 2001, 70.1: 281-312). DAG is one of the key intracellular second messengers that recruits and activates many downstream effectors including protein kinase C (PKC), protein kinase D (PKD) families, and Ras guanyl nucleotide releasing proteins (RasGRPs), which in turn activates NF-κB and extracellular regulated kinase (ERK) pathways (Mdrida, Isabel, et al. Biochemical Journal. 2008, 409.1: 1-18, Joshi, Rohan P., et al. International Journal of Molecular Sciences. 2013, 14.4: 6649-6673). By consuming DAG, DGK controls and tunes the threshold and duration of DAG mediated signaling. Mammalian DGK family comprises 10 different members, in which DGKα, DGKζ and DGKδ are the three major isoforms that are abundantly expressed in lymphoid tissues (Joshi, Rohan P., et al. International Journal of Molecular Sciences. 2013, 14.4: 6649-6673).

Cancer immunotherapy is a type of cancer treatment to manipulate and boost host immune system to recognize and attack cancer cells. A vast majority of studies have focused on targeting immune checkpoint inhibitors, such as CTLA-4 and PD-1/PD-L1, to reinvigorate exhausted CD8⁺ T cells within tumor sites. It was emerged that peripheral T cell tolerance, which under normal circumstances prevents detrimental autoimmune disease, can be hijacked by tumors to prevent anti-tumor immune response during carcinogenesis (Nüssing, Simone, et al. Frontiers in Immunology. 2020, 11: 2461). T cell anergy is one of the most important mechanisms of T cell tolerance and has been reported to occur in tumor infiltrated T cells, which contributes to the immunosuppressive nature of tumor microenvironment (Abe, Brian T., and Fernando Macian. Oncoimmunology. 2013, 2.2: e22679). Anergy-associated transcription factor early growth response gene2 (Egr2) directly binds to Dgka and Dgkz promoter and increases their expression (Zheng, Yan, et al. Journal of Experimental Medicine 2012, 209.12: 2157-2163; Zheng, Yan, et al. Molecular Immunology. 2013, 55.3-4: 283-291). In anergic T cells, both DGKα and DGKζ play critical roles to negatively regulate DAG-signaling downstream of TCR and reduce the strength of TCR activation (Chen, Shelley S., et al. Frontiers in Cell and Developmental Biology. 2016, 4: 130). Thus, immune cell expressed DGKα and DGKζ were investigated as potential targets to reverse the hyporesponsiveness of the tumor infiltrated T cells. It was demonstrated that genetic deletion of DGKα or DGK enhanced cytokine production and proliferation of T cells (Olenchock, Benjamin A., et al. Nature immunology. 2006, 7.11: 1174-1181; Zhong, Xiao-Ping, et al. Nature immunology. 2003, 4.9: 882-890). DGKα or DGKζ single knockout in both mouse or human chimeric antigen receptor (CAR)-T cells showed superior effector function as determined by enhanced in vitro cytotoxicity and cytokine secretion when cocultured with antigen expressing titled cells (Riese, Matthew J., et al. Cancer Research. 2013, 73.12: 3566-3577; Jung, In-Young, et al. Cancer Research. 2018, 78.16: 4692-4703). MesoCAR-transduced DGKα or DGKζ deficient T cells also showed significantly elevated in vivo activity against mesotheliomas (Riese, Matthew J., et al. Cancer Research. 2013, 73.12: 3566-3577). DGKζ^{-/-} mice showed enhanced tumor suppressive efficacy with both orthotopic and subcutaneously implanted models (Wesley, Erin M., et al. Immunohorizons. 2018, 2.4: 107-118; Wee, Susan, et al. AACR; Cancer Res 2019; 79 (13 Suppl): Abstract nr 936). Besides the T cell regulatory function, both DGKα and DGKζ also involve in tuning NK cell activation at tumor site (Prinz, Petra U., et al. International Journal of Cancer. 2014. 135.8: 1832-1841; Yang, Enjun, et al. The Journal of Immunology. 2016, 197.3: 934-941). In addition, DGKζ were found to play a critical role to control the activation threshold of mature B cells (Wheeler, Matthew L., et al. Science Signaling. 2013, 6.297: ra91-ra91). In summary, all these preclinical data suggest titled inhibition of DGKα and DGKζ could be therapeutically beneficial to promote immunity against cancer.

Although the existing anti-CTLA-4 and anti-PD-1 therapies have shown clear clinical benefits in a subset of patients with various tumor types, there are still unmet medical needs to develop novel immunotherapies to achieve robust and durable clinical anti-tumor efficacy. Pre-clinical data strongly suggests there is a great potential of developing DGKα and DGKζ targeted therapies to improve antitumor immunity.

### SUMMARY OF THE DISCLOSURE

The above needs have been met by providing the compounds disclosed herein which have a novel core structure and show the desired inhibition of DGKα and DGKζ. In some embodiments, the compounds disclosed herein show the dual inhibitory activity of both DGKα and DGKζ. In some embodiments, the compounds disclosed herein show the selective inhibitory activity of DGKα over DGKζ. In some embodiments, the compounds disclosed herein show the selective inhibitory activity of DGKζ over DGKα.

Disclosed herein provides a compound of formula (I),

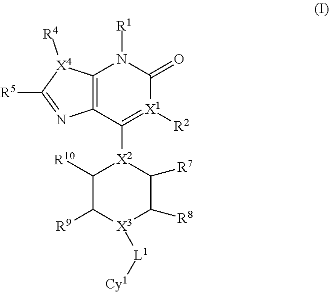

or a pharmaceutically acceptable salt, a tautomer, a stereoisomer, an enantiomer, an isotopologue, or a prodrug thereof, wherein the variables are defined as herein.

In some embodiments, the compound of formula (I) is a compound of formula (IA1); a compound of formula (IA2); a compound of formula (IA3); or a compound of formula (IA4):

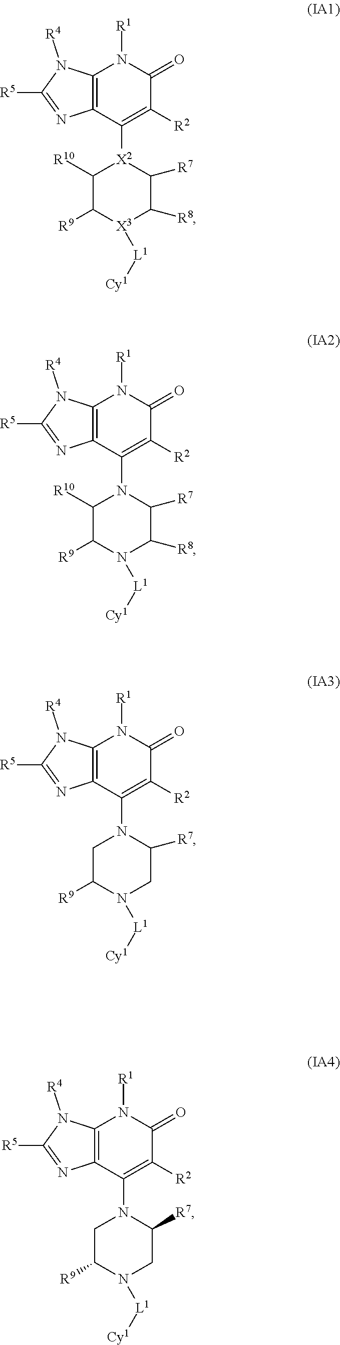

wherein the variables are defined as herein.

In some embodiments, the compound of formula (I) is a compound of formula (IB1); a compound of formula (IB2); a compound of formula (IB3); or a compound of formula (IB4):

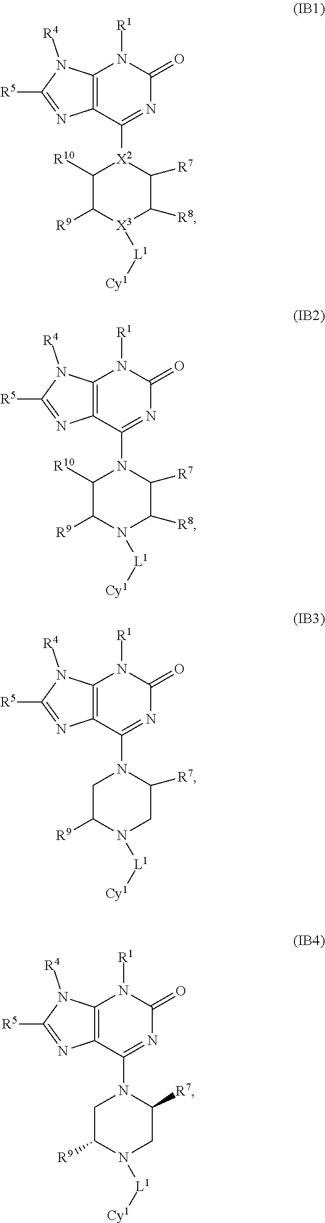

wherein the variables are defined as herein.

In some embodiments, the compound of formula (I) is a compound of formula (IC1); a compound of formula (IC2); a compound of formula (IC3); or a compound of formula (IC4):

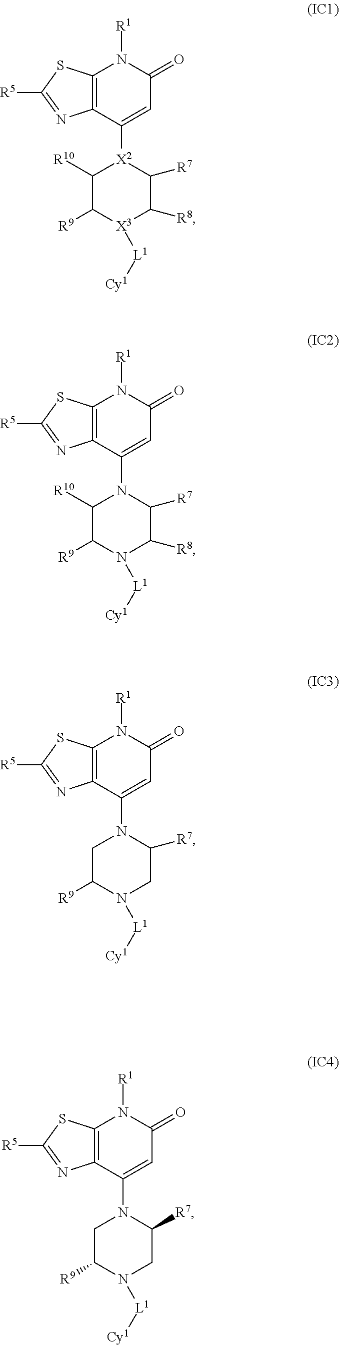

wherein the variables are defined as herein.

In some embodiments, the compound of formula (I) is a compound of formula (II):

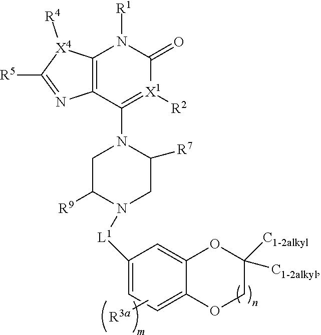

wherein m is 0, 1, 2 or 3, and n is 0 or 1, and the other variables are defined as herein.

In one aspect, provided herein are methods for inhibiting a kinase, for example, DGKα, DGKζ, or both, in a cell expressing said kinase, comprising contacting said cell with an effective amount of a compound as described herein. In one aspect, provided herein are methods for treating diseases of mammals, including humans, in particular hyperproliferative disorders, such as cancer.

In another aspect provided herein are methods for preparing compounds as described herein.

The present embodiments can be understood more fully by reference to the detailed description and examples, which are intended to exemplify non-limiting embodiments.

### DETAILED DESCRIPTION OF THE DISCLOSURE

### Definitions

The following terms have the indicated meanings throughout the specification:

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups derived from an alkane by removal of one hydrogen atom from the same carbon atom, which comprises from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups. The alkyl group can be optionally enriched in deuterium, e.g., —CD₃, —CD₂CD₃ and the like. The term “alkylene” refers to a hydrocarbon group selected from linear and branched saturated hydrocarbon groups derived from an alkane by removal of two hydrogen atoms from the same carbon atom, which comprises from 1 to 6, such as from 1 to 4, carbon atoms, further such as from 1 to 3, more further such as 1, 2 or 3 carbon atoms, include, but not limited to, methylene (—CH₂—), ethylene (—CH₂CH₂—), 1-methymethylene (—CH(CH₃)—), or trimethylene (—CH₂CH₂CH₂—). When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, O(alkyl)aminocarbonyl, aryl, heterocyclyl, or heteroaryl.

The term “halogen” refers to fluoro (F), chloro (Cl), bromo (Br) and iodo (I).

The term “haloalkyl” refers to an alkyl group in which one or more hydrogen is/are replaced by one or more halogen atoms such as fluoro, chloro, bromo, and iodo. Examples of the haloalkyl include haloC₁₋₆alkyl, haloC₁₋₆alkyl or halo C₁₋₄alkyl, but not limited to —CF₃, —CH₂Cl, —CH₂CF₃, —CCl₂, CF₃, and the like.

The term “alkyloxy” or “alkoxy” refers to an alkyl group as defined above attached to the parent molecular moiety through an oxygen atom. Examples of an alkyloxy, e.g., C₁₋₆alkyloxy or C₁₋₄ alkyloxy include, but not limited to, methoxy, ethoxy, isopropoxy, propoxy, n-butoxy, tert-butoxy, pentoxy and hexoxy and the like.

The term “amino” refers to —NH₂.

The term “alkenyl” herein refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C2-6 alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” herein refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C2-6 alkynyl, include, but not limited to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl. For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. In particular, Examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “deuterated compound” refers to a compound wherein one or more carbon-bound hydrogen(s) are replaced by one or more deuterium(s). The term “deuterated” is used herein to modify a chemical structure or an organic group or radical, wherein one or more carbon-bound hydrogen(s) are replaced by one or more deuterium(s), e.g., “deuterated-alkyl”, “deuterated-cycloalkyl”, “deuterated-heterocycloalkyl”, “deuterated-aryl”, “deuterated-morpholinyl”, and the like. For example, the term “deuterated-alkyl” defined above refers to an alkyl group as defined herein, wherein at least one hydrogen atom bound to carbon is replaced by a deuterium. In a deuterated alkyl group, at least one carbon atom is bound to a deuterium; and it is possible for a carbon atom to be bound to more than one deuterium; it is also possible that more than one carbon atom in the alkyl group is bound to a deuterium.

The term “aryl” used alone or in combination with other terms refers to an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like). In some embodiments, an aryl group refers to a group selected from: 5- and 6-membered carbocyclic aromatic rings, e.g., phenyl; bicyclic ring systems such as 7 to 12-membered bicyclic ring systems, wherein at least one ring is carbocyclic and aromatic, e.g., naphthyl and indanyl; and, tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “heteroaryl” herein refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from nitrogen, oxygen or optionally oxidized sulfur as ring member(s), with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

The term “optionally oxidized sulfur” used herein refers to —S—, SO or SO₂.

The terms “aromatic heterocyclic ring” and “heteroaryl” are used interchangeably throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is a 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) 1H-pyrazolyl (such as 1H-pyrazol-3-yl, 1H-pyrazol-4-yl or 1H-pyrazol-5-yl), pyridyl or pyridinyl (such as 2-pyridyl, 3-pyridyl, or 4-pyridyl), cinnolinyl, pyrazinyl, pyrimidinyl (such as pyrimidin-2-yl, pyrimidin-4-yl, pyrimidin-5-yl or 2,4-pyrimidinyl, 3,5-pyrimidinyl), imidazolyl (such as 1H-imidazol-2-yl, 1H-imidazol-4-yl, 1H-imidazol-5-yl, or 2,4-imidazolyl), imidazopyridinyl, isoxazolyl, oxazolyl, thiazolyl, isothiazolyl, thiadiazolyl (such as 1,2,3-thiadiazolyl, 1,2,4-thiadiazolyl, or 1,3,4-thiadiazolyl), tetrazolyl, thienyl (such as thien-2-yl, thien-3-yl), triazinyl, benzothienyl, furyl or furanyl, benzofuryl, benzoimidazolyl, indolyl (such as 1H-indol-2-yl, 1H-indol-3-yl, 1H-indol-4-yl, 1H-indol-5-yl, 1H-indol-6-yl or 1H-indol-7-yl), isoindolyl, indolinyl, oxadiazolyl (such as 1,2,3-oxadiazolyl, 1,2,4-oxadiazolyl, or 1,3,4-oxadiazolyl), phthalazinyl, pyrazinyl (such as pyrazin-2-yl), pyridazinyl, pyrrolyl, triazolyl (such as 1,2,3-triazolyl, 1,2,4-triazolyl, or 1,3,4-triazolyl), quinolinyl (such as quinolin-2-yl, quinolin-3-yl, quinolin-4-yl, quinolin-5-yl, quinolin-6-yl, or quinolin-7-yl), isoquinolinyl (such as isoquinolin-1-yl, isoquinolin-3-yl, isoquinolin-4-yl, isoquinolin-5-yl, isoquinolin-6-yl, isoquinolin-7-yl, or isoquinolin-8-yl), pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl (such as furazan-2-yl, furazan-3-yl), benzofurazanyl, benzothiophenyl, benzothiazolyl, benzoxazolyl (such as benzo[d]oxazol-2-yl, benzo[d]oxazol-4-yl, benzo[d]oxazol-5-yl, benzo[d]oxazol-6-yl or benzo[d]oxazol-7-yl), quinazolinyl, quinoxalinyl (such as quinoxalin-2-yl, quinoxalin-3-yl, quinoxalin-4-yl, quinoxalin-5-yl, quinoxalin-6-yl, quinoxalin-7-yl or quinoxalin-8-yl), naphthyridinyl (such as 1,8-naphthyridin-2-yl, 1,8-naphthyridin-3-yl, or 1,8-naphthyridin-4-yl), 2,3-dihydro-[1,4]dioxino[2,3-b]pyridinyl (such as 2,3-dihydro-[1,4]dioxino[2,3-b]pyridin-6-yl, 2,3-dihydro-[1,4]dioxino[2,3-b]pyridin-7-yl, or 2,3-dihydro-[1,4]dioxino[2,3-b]pyridin-8-yl), furopyridinyl, benzothiazolyl (such as benzo[d]thiazol-2-yl, benzo[d]thiazol-4-yl, benzo[d]thiazol-5-yl, benzo[d]thiazol-6-yl or benzo[d]thiazol-7-yl), benzo[d]imidazolyl (such as 1H-benzo[d]imidazol-2-yl, 1H-benzo[d]imidazol-4-yl, 1H-benzo[d]imidazol-5-yl, 1H-benzo[d]imidazol-6-yl or 1H-benzo[d]imidazol-7-yl), [1,2,4]triazolo[1,5-a]pyridinyl (such as [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, or [1,2,4]triazolo[1,5-a]pyridin-8-yl), 3H-imidazo[4,5-b]pyridinyl (such as 3H-imidazo[4,5-b]pyridin-2-yl, 3H-imidazo[4,5-b]pyridin-5-yl, 3H-imidazo[4,5-b]pyridin-6-yl or 3H-imidazo[4,5-b]pyridin-7-yl), 1H-imidazo[4,5-b]pyridinyl (such as 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl), [1,2,4]triazolo[1,5-a]pyridinyl (such as [1,2,4]triazolo[1,5-a]pyridin-2-yl, 1,2,4]triazolo[1,5-a]pyridin-5-yl, [1,2,4]triazolo[1,5-a]pyridin-6-yl, [1,2,4]triazolo[1,5-a]pyridin-7-yl or [1,2,4]triazolo[1,5-a]pyridin-8-yl), indazolyl (such as 1H-indazol-5-yl), 5,6,7,8-tetrahydroisoquinoline, thiazolo[5,4-b]pyridinyl (such as thiazolo[5,4-b]pyridin-2-yl, thiazolo[5,4-b]pyridin-5-yl, thiazolo[5,4-b]pyridin-6-yl or thiazolo[5,4-b]pyridin-7-yl), thiazolo[4,5-b]pyridinyl (such as thiazolo[4,5-b]pyridin-2-yl, thiazolo[4,5-b]pyridin-5-yl, thiazolo[4,5-b]pyridin-6-yl or thiazolo[4,5-b]pyridin-7-yl), thieno[2,3-b]pyridinyl (such as thieno[2,3-b]pyridin-2-yl, thieno[2,3-b]pyridin-3-yl, thieno[2,3-b]pyridin-4-yl, thieno[2,3-b]pyridin-5-yl or thieno[2,3-b]pyridin-6-yl), thieno[3,2-b]pyridinyl (such as thieno[3,2-b]pyridin-2-yl, thieno[3,2-b]pyridin-3-yl, thieno[3,2-b]pyridin-5-yl, thieno[3,2-b]pyridin-6-yl, or thieno[3,2-b]pyridin-7-yl).

Also, a “heteroaryl” fused with a “Heterocyclyl” is defined as “heteroaryl”.

“Heterocyclyl,” “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “monocyclic heterocyclyl” refers to monocyclic groups in which at least one ring member is a heteroatom selected from nitrogen, oxygen or optionally oxidized sulfur. A heterocycle may be saturated or partially saturated.

Exemplary monocyclic 4 to 9-membered heterocyclyl groups include, but not limited to, (as numbered from the linkage position assigned priority 1) pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2,5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, oxetan-3-yl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, or 1,1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/4-membered, 3-membered/5-membered, 4-membered/5-membered, 4-membered/6-membered, 5-membered/5-membered, or 5-membered/6-membered mono-spiro heterocyclyl. Examples of spiro heterocyclyl groups include, but not limited to, spiro[2.2]pentanyl, spiro[2.3]hexanyl, spiro[2.4]heptanyl, spiro[3.3]heptanyl, spiro[2.5]octanyl, spiro[3.4]octanyl, spiro[2.6]nonanyl, spiro[3.5]nonanyl, spiro[4.4]nonanyl, spiro[2.7]decanyl, spiro[3.6]decanyl, spiro[4.5]decanyl, spiro[3.7]undecanyl, spiro[4.6]undecanyl, spiro[5.5]undecanyl, spiro[4.7]dodecanyl, and spiro[5.6]dodecanyl, wherein one or two carbon atoms are replaced with oxygen, or nitrogen, for example, 2-oxa-6-azaspiro[3.3]heptanyl (e.g., 2-oxa-6-azaspiro[3.3]heptan-6-yl), 1,7-dioxaspiro[4.5]decyl, 2-oxa-7-aza-spiro[4.4]nonyl, 7-oxa spiro[3.5]nonyl and 5-oxa-spiro[2.4]heptyl, spiro[benzo[d][1,3]dioxole-2,1′-cyclobutan]-yl (e.g., spiro[benzo[d][1,3]dioxole-2,1′-cyclobutan]-5-yl).

The term “fused heterocyclic group” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but none of the rings has a completely conjugated pi-electron system. Preferably, a fused heterocyclyl is 6 to 14-membered, and more preferably 7 to 10-membered. According to the number of membered rings, a fused heterocyclyl is divided into bicyclic, tricyclic, tetracyclic, or polycyclic fused heterocyclyl, preferably refers to bicyclic or tricyclic fused heterocyclyl, and more preferably 5-membered/5-membered, or 5-membered/6-membered bicyclic fused heterocyclyl. Representative examples of fused heterocycles include, but not limited to, the following groups octahydrocyclopenta[c]pyrrole (e.g., octahydrocyclopenta[c]pyrrol-2-yl), octahydropyrrolo[3,4-c]pyrrolyl, octahydroisoindolyl, isoindolinyl (e.g., isoindoline-2-yl), octahydro-benzo[b][1,4]dioxin.

The term “bridged heterocyclyl” refers to a 5- to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but not limited to, the following groups: 2-azabicyclo[2.2.1]heptyl, azabicyclo[3.1.0]hexyl, 2-azabicyclo[2.2.2]octyl and 2-azabicyclo[3.3.2]decyl.

A “cycloalkylalkyl” group is a radical of the formula: -alkyl-cycloalkyl, wherein alkyl and cycloalkyl are as defined above. Substituted cycloalkylalkyl groups may be substituted at the alkyl, the cycloalkyl, or both the alkyl and the cycloalkyl portions of the group. Representative cycloalkylalkyl groups include but are not limited to methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” group is —O-(alkyl), wherein alkyl is defined above.

An “alkoxyalkyl” group is -(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R^{#}) or —C(O)NH₂, wherein each R^{#} is independently a substituted or unsubstituted alkyl, cycloalkyl, aryl, aralkyl, heterocyclyl or heterocyclyl group as defined herein.

An “acylamino” group is a radical of the formula: —NHC(O)(R^{#}) or —N(alkyl)C(O)(R^{#}), wherein each alkyl and Rare independently as defined above.

A “sulfonylamino” group is a radical of the formula: —NHSO₂(R^{#}) or —N(alkyl)SO₂(R^{#}), wherein each alkyl and Rare defined above.

A “urea” group is a radical of the formula: —N(alkyl)C(O)N(R^{#})₂, —N(alkyl)C(O)NH(R^{#}), —N(alkyl)C(O)NH₂, —NHC(O)N(R^{#})₂, —NHC(O)NH(R^{#}), or —NH(CO)NHR^{#}, wherein each alkyl and R^{#} are independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, orthiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the titled stereoisomer contains no more than 35%, such as no more than 30%, further such as no more than 25%, even further such as no more than 20%, by weight of any other stereoisomer(s). In some embodiments, the term “substantially pure” means that the titled stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by flash column chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

“Selective inhibitory activity” or “selectivity” refers to the difference in the degree of inhibition against DGKα and DGKζ; In some embodiments, “a compound showing selective inhibitory activity of DGKα over DGKζ” refers a compound which shows a ratio of IC50 against DGKζ and IC50 against DGKα larger than or equal to about 20. In some embodiments, “a compound showing selective inhibitory activity of DGKζ over DGKα” refers a compound which shows a ratio of IC50 against DGKα and IC50 against DGKζ larger than or equal to about 20. In some embodiments, “a compound showing selective inhibitory activity of DGKα over DGKζ” refers a compound which shows an IC50 against DGKα is not larger than about 2000 nM with the ratio of IC50 against DGKζ and IC50 against DGKα larger than or equal to about 20; “a compound showing selective inhibitory activity of DGKζ over DGKα” refers a compound which shows an IC50 against DGKζ is not larger than about 2000 nM with the ratio of IC50 against DGKα and IC50 against DGKζ larger than or equal to about 20; and “a compound showing dual inhibitory activity” refers to a compound which shows inhibitory activities against both DGKα and DGKζ with IC50 no larger than 500 nM and the ratio of the two IC50 values no more than 20. In some embodiments, “a compound showing dual inhibitory activity” refers to a compound which shows inhibitory activities against both DGKα and DGKζ with IC50 no larger than 1000 nM and the ratio of the two IC50 values no more than 20. In some embodiments, “a compound showing dual inhibitory activity” refers to a compound which shows inhibitory activities against both DGKα and DGKζ with IC50 no larger than 2000 nM and the ratio of the two IC50 values no more than 20.

As defined herein, “a pharmaceutically acceptable salt thereof” include salts of at least one compound of Formulas (I), (IA1), (IA2), (IA3), (IA4), (IB1), (IB2), (IB3), (IB4), (IC1), (IC2), (IC3), (IC4) and (II) (collectively referred to as formulas (I) to (II)), and salts of the stereoisomers of the compound of Formulas (I) to (II), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

All Formulations of the pharmaceutical composition disclosed herein can be produced by the conventional methods in the pharmaceutical field. For example, the active ingredient can be mixed with one or more excipients, then to make the desired Formulation. The “pharmaceutically acceptable excipient” refers to conventional pharmaceutical carriers suitable for the desired pharmaceutical Formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the claims which follow, unless the context requires otherwise, the term “comprise,” and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cn-m” indicates a range which includes the endpoints, wherein n and m are integers and indicate the number of carbons. Examples include C₁₋₈, C₁₋₆, and the like.

The term “Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

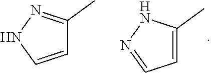

As readily understood by one skilled in the art, a wide variety of functional groups and other structures may exhibit tautomerism and all tautomers of compounds of any one of formulas (I) to (II) are within the scope of the present invention.

Unless specifically defined elsewhere in this document, all other technical and scientific terms used herein have the meaning commonly understood by one of ordinary skill in the art to which this invention belongs.

It should also be noted the compounds provided herein can contain unnatural proportions of atomic isotopes at one or more of the atoms. For example, the compounds may be radiolabeled with radioactive isotopes, such as for example tritium (H), iodine-125 (¹²⁵I), sulfur-35 (³⁵S), or carbon-14 (⁴C), or may be isotopically enriched, such as with deuterium (H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

The term “DGK” or “DAGK” (Diacylglycerol kinase) refers to a family of enzymes that catalyzes the conversion of diacylglycerol (DAG) to phosphatidic acid (PA), utilizing ATP as a source of the phosphate. In non-stimulated cells, DGK activity is low, allowing DAG to be used for glycerophospholipid biosynthesis, but on receptor activation of the phosphoinositide pathway, DGK activity increases, driving the conversion of DAG to PA. As both lipids are thought to function as bioactive lipid signaling molecules with distinct cellular targets, DGK therefore occupies an important position, effectively serving as a switch by terminating the signalling of one lipid while simultaneously activating signalling by another. See Mdrida I, Avila-Flores A, Merino E (January 2008). “Diacylglycerol kinases: at the hub of cell signalling,” The Biochemical Journal, 409 (1): 1-18. Currently, ten members of the DGK family have been cloned and identified. Although all family members have conserved catalytic domains and two cysteine rich domains, they are further classified into five groups according to the presence of additional functional domains and substrate specificity. See van Blitterswijk W J, Houssa B (October 2000), “Properties and functions of diacylglycerol kinases,” Cellular Signalling, 12 (9-10): 595-605. These are as follows:
a) Type 1—DGK-α, DGK-β, DGK-γ—contain EF-hand motifs and a recoverin homology domain
b) Type 2—DGK-δ, DGK-η, DGK-κ—contain a pleckstrin homology domain
c) Type 3—DGK-ε—has specificity for arachidonate-containing DAG
d) Type 4—DGK-ζ, DGK-t—contain a MARCKS homology domain, ankyrin repeats, a C-terminal nuclear localisation signal, and a PDZ-binding motif.
e) Type 5—DGK-θ—contains a third cysteine-rich domain, a pleckstrin homology domain and a proline rich region.

The term “treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, “treating” means and alleviation, in whole or in part, of a disorder, disease or condition, or symptoms associated with a condition, treatable or preventable by inhibition of a DGK pathway. In another embodiment, “treating” means and alleviation, in whole or in part, of a disorder, disease or condition, or symptoms associated with a condition, treatable or preventable by inhibition of a DGK-α pathway. In another embodiment, “treating” means and alleviation, in whole or in part, of a disorder, disease or condition, or symptoms associated with a condition, treatable or preventable by inhibition of a DGK-ζ pathway. In one embodiment, the disorder is cancer.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In another, the disorder is a condition, treatable or preventable by inhibition of a DGK pathway. In another, the disorder is a condition, treatable or preventable by inhibition of a DGK-α pathway. In another, the disorder is a condition, treatable or preventable by inhibition of a DGK-ζ pathway. In one embodiment, the disorder is cancer.

The term “subject” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having liver fibrotic disorders or diabetes or metabolic syndrome leading to liver fibrotic disorders, or a condition, treatable or preventable by inhibition of a JNK pathway, or a symptom thereof.

As used herein, the term “Compound” refers to compounds of formulas (I), (IA1), (IA2), (IA3), (IA4), (IB1), (IB2), (IB3), (IB4), (IC1), (IC2), (IC3), (IC4), (II) and salts of the stereoisomers of the compound of Formulas (I), (IA1), (IA2), (IA3), (IA4), (IB1), (IB2), (IB3), (IB4), (IC1), (IC2), (IC3), (IC4) and (II) (collectively referred to as formulas (I) to (II)) as well as to further embodiments provided herein. In one embodiment, a “Compound” is a compound exemplified herein. The term “Compound” includes pharmaceutically acceptable salts, tautomers, isotopologues, stereoisomers, deuterated compounds and prodrugs of the compounds provided herein.

As used herein and unless otherwise indicated, the term “prodrug” means a compound that can hydrolyze, oxidize, or otherwise react under biological conditions (in vitro or in vivo) to provide an active compound, particularly a compound of any one of formulas (I) to (II). Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a Compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

### Compounds

The compounds provided herein have a novel core structure and show the desired inhibition of DGKα and DGKζ. In some embodiments, the compounds disclosed herein show the dual inhibitory activity of both DGKα and DGKζ. In some embodiments, the compounds disclosed herein show the selective inhibitory activity of DGKα over DGKζ. In some embodiments, the compounds disclosed herein show the selective inhibitory activity of DGKζ over DGKα.

Disclosed herein provides a compound of formula (I),

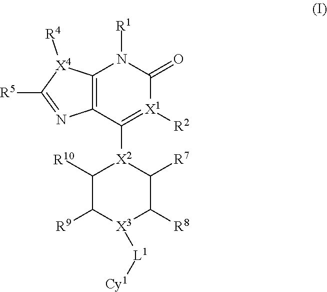

or a pharmaceutically acceptable salt, a tautomer, a stereoisomer, an enantiomer, an isotopologue, or a prodrug thereof,
wherein
X¹ is C or N,
each of X² and X³ is independently selected from —N— or —CH—;
X⁴ is N, O or S;
R¹ is hydrogen, deuterium, halogen, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl; R² is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl, provided that R² is absent when X¹ is N;
R⁴ is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl, provided that R⁴ is absent when X⁴ is O or S;
R⁵ is hydrogen, halogen, cyano, substituted or unsubstituted alkyl, substituted or unsubstituted alkoxyl, substituted or unsubstituted alkenyl, substituted or unsubstituted alkynyl, substituted or unsubstituted cycloalkyl, substituted or unsubstituted aryl, substituted or unsubstituted heterocyclyl, or substituted or unsubstituted heteroaryl, R^{5a}—C(O)—, R^{5a}—C(O)O—, R^{5a}—O—C(O)—, R^{5a}—C(O)NR^{5b}—, R^{5a}—NR^{5b}—C(O)—, or R^{5a}—SO₂—, wherein R^{5a} and R^{5b} are each independently hydrogen, alkyl, or cycloalkyl;
each of R⁷, R⁹, R⁸, and R¹⁰ is independently hydrogen, cyano, substituted or unsubstituted alkyl, substituted or unsubstituted alkoxyl, substituted or unsubstituted cycloalkyl, —C(O)R^{7a}, or -alkyl-C(O)R^{7a}, and wherein R^{a} is hydrogen, alkyl, or alkoxy, provided that at least one of R⁷ and R⁹ is not hydrogen; or R⁷ and R⁹ are each hydrogen and R⁹ and R¹⁰ together form a bridge containing at least one —CH₂-moiety in addition to the two bridgehead atoms; or R⁸ and R¹⁰ are each hydrogen and R⁷ and R⁹ together form a bridge containing at least one —CH₂— moiety in addition to the two bridgehead atoms;
L¹ is a direct bond, —O—, —N(R^{L})—, substituted or unsubstituted alkyl, -alkylene or —C(O)—, wherein R^{L} is hydrogen or alkyl;
Cy¹ is unsubstituted or substituted cycloalkyl, unsubstituted or substituted aryl, unsubstituted or substituted heterocyclyl, or unsubstituted or substituted heteroaryl, wherein Cy¹ is optionally substituted with 1 to 5 substituents R^{3a};
wherein R^{3a} is selected from deuterium, alkoxy, alkyl, halogen, oxo, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, cycloalkyl or heterocyclyl; optionally wherein two R^{3a} connect to the same carbon and together form a spirocyclic ring; optionally wherein two R^{3a} form a fused ring with Cy¹, wherein R^{3a} is unsubstituted or substituted with cyano, alkoxy, alkyl, halogen, or hydroxy; and
wherein R^{3b} and R^{3c} are each independently hydrogen or alkyl.
   The definitions of X¹ and X⁴

In some embodiments, X¹ is C. In some embodiments, X¹ is N. In some embodiments, X⁴ is N. In some embodiments, X⁴ is S. In some embodiments, X⁴ is O. In some embodiments, X¹ is C and X⁴ is N. In some embodiments, X¹ is N and X⁴ is N. In some embodiments, X¹ is C and X⁴ is S.

In some embodiments, the compound of formula (I) is a compound of any one of formulas (IA1), (IB1) or (IC1):

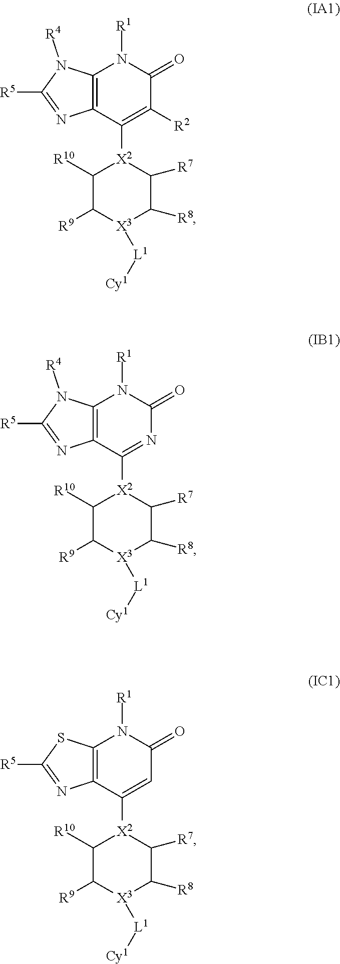

wherein the variables are defined as herein.

In some embodiments, the compound of formula (I) is a compound of any one of formula (IA2), (IB2) or (IC2):

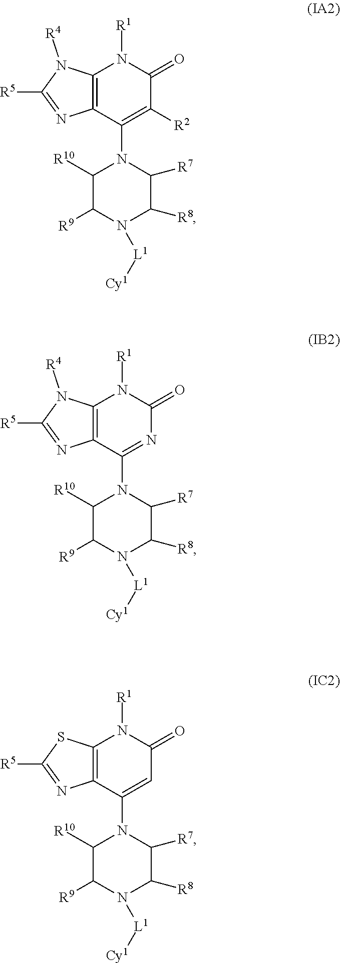

wherein the variables are defined as herein.

In some embodiments, the compound of formula (I) is a compound of any one of formula (IA3), (IB3) or (IC3):

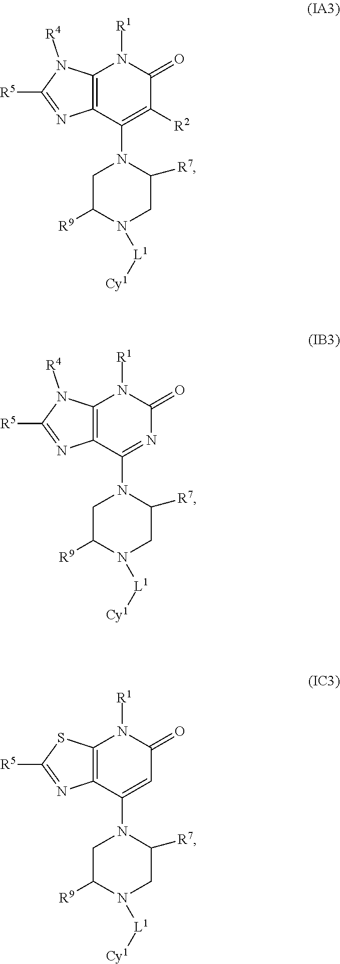

wherein the variables are defined as herein.

In some embodiments, the compound of formula (I) is a compound of any one of formula (IA4), (IB4) or (IC4):

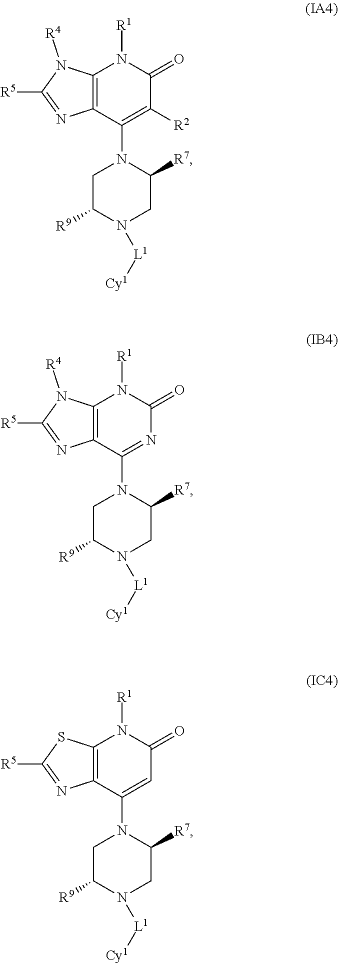

wherein the variables are defined as herein.

In some embodiments, the compound of formula (I) is a compound of formula (II):

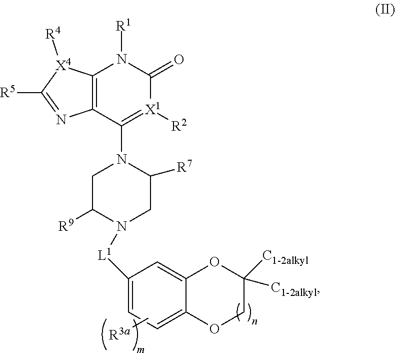

or a pharmaceutically acceptable salt, a tautomer, a stereoisomer, an enantiomer, an isotopologue, or a prodrug thereof,
wherein
X¹ is C or N,
X⁴ is N, O or S;
R¹ is hydrogen, deuterium, halogen, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl;
R² is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl, provided that R² is absent when X¹ is N;
R⁴ is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl, provided that R⁴ is absent when X⁴ is O or S;
R⁵ is hydrogen, halogen, cyano, substituted or unsubstituted alkyl, substituted or unsubstituted alkoxyl, substituted or unsubstituted alkenyl, substituted or unsubstituted alkynyl, substituted or unsubstituted cycloalkyl, substituted or unsubstituted aryl, substituted or unsubstituted heterocyclyl, or substituted or unsubstituted heteroaryl, R^{5a}—C(O)—, R^{5a}—C(O)O—, R^{5a}—O—C(O)—, R^{5a}—C(O)NR^{5b}—, R^{5a}—NR^{5b}—C(O)—, or R^{5a}—SO₂—, wherein R^{5a} and R^{5b} are each independently hydrogen, alkyl, or cycloalkyl;
each of R⁷, and R⁹ is independently hydrogen, cyano, substituted or unsubstituted alkyl, substituted or unsubstituted alkoxyl, substituted or unsubstituted cycloalkyl, —C(O)R^{7a}, or -alkyl-C(O)R^{7a}, and wherein R^{7a} is hydrogen, alkyl, or alkoxy, provided that at least one of R⁷ and R⁹ is not hydrogen;
or R⁷ and R⁹ together form a bridge containing at least one —CH₂— moiety in addition to the two bridgehead atoms;
L¹ is a direct bond, —O—, —N(R^{L})—, substituted or unsubstituted alkyl, -alkylene or —C(O)—, wherein R^{L} is hydrogen or alkyl;
m is 0, 1, 2 or 3,
n is 0 or 1
wherein R^{3a} is selected from deuterium, alkoxy, alkyl, halogen, oxo, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, cycloalkyl or heterocyclyl;
optionally wherein two R^{3a} connect to the same carbon and together form a spirocyclic ring;
optionally wherein two R^{3a} form a fused ring with Cy¹, wherein R^{3a} is unsubstituted or substituted with cyano, alkoxy, alkyl, halogen, or hydroxy; and
wherein R^{3b} and R^{3c} are each independently hydrogen or alkyl.

Unless specifically indicated otherwise, “embodiment” or “embodiments” hereinafter refer to embodiments concerning a compound of any one of formulas (I) to (II).

### The Definitions of R¹

In some embodiments, R¹ is hydrogen, or substituted or unsubstituted alkyl. In some embodiments, R¹ is hydrogen, or substituted or unsubstituted C₁₋₄alkyl.

In some embodiments, R¹ is hydrogen, or C₁₋₄alkyl optionally substituted with deuterium, halogen, hydroxy, alkoxy or cycloalkyl. In some embodiments, R¹ is hydrogen, or C₁₋₄alkyl optionally substituted with deuterium, halogen, hydroxy, alkoxy or cycloalkyl. In some embodiments, R¹ is hydrogen, or C₁₋₃alkyl optionally substituted with deuterium, or halogen. In some embodiments, R¹ is hydrogen, or C₁₋₃alkyl optionally substituted with deuterium.

In some embodiments, R¹ is hydrogen, methyl, methyl-d3, ethyl, isopropyl, 2-hydroxyethyl, 2-methoxyethyl, 2,2,2-trifluoroethyl, 2,2-difluoroethyl, or cyclopropylmethyl. In some embodiments, R¹ is hydrogen, methyl, ethyl or methyl-d3. In some embodiments, R¹ is methyl or methyl-d3. In some embodiments, R¹ is methyl.

### The Definitions of R²

In some embodiments, R² is hydrogen, halogen, alkyl, alkoxyl, or cyano, provided that R² is absent when X¹ is N. In some embodiments, R² is hydrogen, halogen, C₁₋₄alkyl, C₁₋₄ alkoxyl or cyano. In some embodiments, R² is hydrogen, F, Br, Cl or CN. In some embodiments, R² is hydrogen. In some embodiments, R² is F, Br, or Cl.

In some embodiments, R² is hydrogen, halogen, C₁₋₄alkyl, C₁₋₄ alkoxyl or cyano; preferably R² is hydrogen, F, Br, Cl or CN; more preferably R² is hydrogen, F, or CN; even more preferably R² is hydrogen.

### The Definitions of R⁴

In some embodiments, R⁴ is hydrogen, halogen or alkyl, wherein the alkyl is optionally substituted with deuterium, halogen or —OR^{4a}, wherein R^{4a} is hydrogen, alkyl, alkenyl, alkynyl, cycloalkyl or heterocycloalkyl, wherein alkyl, alkenyl, alkynyl, cycloalkyl or heterocycloalkyl is optionally substituted with —C₁₋₆alkyl, —C₁₋₆alkoxy or —C₃₋₈cycloalkyl. In some embodiments, R⁴ is hydrogen, halogen or C₁₋₄alkyl, wherein the alkyl is optionally substituted with halogen or —OR⁴a. In some embodiments, R⁴ is hydrogen, halogen or C₁₋₄alkyl, wherein the alkyl is optionally substituted with deuterium or halogen. In some embodiments, R⁴ is hydrogen, or C₁₋₃alkyl optionally substituted with deuterium, or halogen. In some embodiments, R⁴ is hydrogen, or C₁₋₃alkyl optionally substituted with deuterium.

In some embodiments, R⁴ is hydrogen, fluoro, chloro, bromo, methyl, methyl-d3, trifluoromethyl, ethyl, 2,2-difluoroethyl, 2,2,2-trifluoroethyl, propyl, cyclopropyl. In some embodiments, R⁴ is hydrogen, methyl, methyl-d3, ethyl, 2,2-difluoroethyl, propyl, or cyclopropyl.

### The Definitions of R⁵

In some embodiments, R⁵ is hydrogen, alkyl, alkenyl, alkynyl, cycloalkyl, R^{5a}—C(O)—, R^{5a}—C(O)O—, R^{5a}—O—C(O)—, R^{5a}—C(O)NR^{5b}—, R^{5a}—NR^{5b}—C(O)—, R^{5a}—SO₂—or heterocyclyl, wherein said alkyl is unsubstituted or substituted with cyano, —C(O)OR^{5c}, —C(O)R^{5c}, —C(O)NR^{5c}R^{5d}, heterocyclyl, alkoxy, hydroxy, cycloalkyl, or NR^{5c}R^{5d}; and wherein each of said cycloalkyl and heterocyclyl is unsubstituted or substituted with alkyl, cyano or halogen substituted alkyl, cyano, —C(O)OR^{5c}, —C(O)R^{5c}, —C(O)NR^{5c}R^{5d} heterocyclyl, alkoxy, hydroxy, cycloalkyl, NR^{5c}R^{5d}, or R^{5c}—SO₂—, wherein R^{5a} and R^{5b} are each independently hydrogen, alkyl, or cycloalkyl; and wherein R^{5c} and R^{5d} are hydrogen or alkyl.

In some embodiments, R⁵ is hydrogen, alkyl, alkenyl or alkynyl, wherein said alkyl is unsubstituted or substituted with cyano. In some embodiments, R⁵ is C₁₋₄alkyl, C₂₋₄alkenyl or C₂₋₄alkynyl, wherein said alkyl is substituted with cyano. In some embodiments, R⁵ is C₁₋₄alkyl, wherein said alkyl is substituted with cyano.

In some embodiments, R⁵ is C₁₋₄alkyl, wherein said alkyl is substituted with cyano, alkoxy, hydroxy, NR^{5c}R^{5d}, ═N—O—CH₃, —S(═O)—CH₃, —S(═O)₂—CH₃, or —S(═O)(=NH)—CH₃.

In some embodiments, R⁵ is hydrogen, —CH₂—CN, —CH₂C(O)—OMe, —CH(CH₃)CN, oxiran-2-ylmethyl-, prop-2-yn-1-yl, but-2-yn-1-yl, prop-1-en-2-yl, methyl, isopropyl, —CH₂CH₂—O—CH₃, —CH₂—O—CH₃, —CH(CH₃)—O—CH₃, —CH₂C(O)NH₂, —CH₂CH₂—OH, —CH₂—OH, cyclopropyl-CH₂—, —CH₂CH₂N(CH₃)₂, CH₃—SO₂—, cyclopropyl, cyclobutyl, cyclopropyl-C(O)—, 1-cyanocyclopropyl, 2-cyanocyclopropyl, 2-cyanocyclobutyl, 3-(cyanomethyl)-1-(ethylsulfonyl)azetidine-3-yl, 1-cyano-2-cyclopentyleth-2-yl, —CH₂—N(CH₃)₂, —CH═NO—CH₃, —CH₂—S(═O)—CH₃, —CH₂—S(═O)₂—CH₃, or —CH₂—S(═O)(=NH)—CH₃; preferably, R is hydrogen, CN—CH₂—, —CH₂C(O)—OMe, —CH(CH₃)CN, oxiran-2-ylmethyl-, prop-2-yn-1-yl, but-2-yn-1-yl, prop-1-en-2-yl, —CH₂—OH, —CH(CH₃)CN, —CH₂—O—CH₃, —CH(CH₃)—O—CH₃, —CH₂—N(CH₃)₂, —CH═N—O—CH₃, —CH₂—S(═O)—CH₃, —CH₂—S(═O)₂—CH₃, or —CH₂—S(═O)(=NH)—CH₃; more preferably, R⁵ is CN—CH₂—, prop-2-yn-1-yl, but-2-yn-1-yl, or prop-1-en-2-yl, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(=NH)—CH₃; more preferably R⁵ is but-2-yn-1-yl, CN—CH₂—, —CH₂—OH, —CH(CH₃)CN, —CH₂—O—CH₃, —CH(CH₃)—O—CH₃, —CH₂—N(CH₃)₂, —CH═NO—CH₃, —CH₂—S(═O)—CH₃, —CH₂—S(═O)₂—CH₃, or —CH₂—S(═O)(=NH)—CH₃; more preferably R⁵ is CN—CH₂—or but-2-yn-1-yl.

The definitions of R⁷/R⁹, R⁸/R¹⁰

In some embodiments, each of R⁷, R⁹, R, and R¹⁰ is independently hydrogen, alkyl, or —C(O)R^{7a}, wherein said alkyl is unsubstituted or substituted with halogen, cyano, hydroxy, or alkoxy, and wherein R^{7a} is hydrogen, alkyl, or alkoxy, provided that at least one of R⁷ and R⁹ is not hydrogen.

In some embodiments, each of R⁷ and R⁹ is independently hydrogen, alkyl, or —C(O)R^{7a}, wherein said alkyl is unsubstituted or substituted with halogen, cyano, hydroxy, or alkoxy, and wherein R^{7a} is hydrogen, alkyl, or alkoxy. In some embodiments, each of R⁷ and R⁹ is independently C₁₋₄alkyl. In some embodiments, each of R⁷ and R⁹ is independently C₁₋₂alkyl.

In some embodiments, R⁷ and R⁹ are each independently hydrogen, methyl, ethyl, methoxymethyl, 1-hydroxyethyl, 2-methoxyethyl, cyanomethyl, hydroxyethyl, hydroxymethyl, methoxycarbonyl, difluoromethyl, provided that at least one of R⁷ and R⁹ is not hydrogen.

In some embodiments, R⁸ and R¹⁰ are each hydrogen.

In some embodiments, R⁷ is methyl, and R⁹ is methyl; or R⁷ is ethyl, and R⁹ is ethyl; or R⁷ is methyl, and R⁹ is ethyl; or R⁷ is methyl, and R⁹ is methoxycarbonyl; or R⁷ is hydrogen, and R⁹ is methyl; or R⁷ is hydrogen, and R⁹ is ethyl.

In some embodiments, R⁷ and R⁹ are each hydrogen and R⁸ and R¹⁰ together form a bridge containing at least one —CH₂— moiety in addition to the two bridgehead atoms. In some embodiments, R⁷ and R⁹ are each hydrogen and R⁸ and R¹⁰ together form a bridge containing one —CH₂—moiety in addition to the two bridgehead atoms. In some embodiments, R⁷ and R⁹ are each hydrogen and R⁸ and R¹⁰ together form a bridge containing two —CH₂—moieties in addition to the two bridgehead atoms.

In some embodiments, R⁸ and R¹⁰ are each hydrogen and R⁷ and R⁹ together form a bridge containing at least one —CH₂— moiety in addition to the two bridgehead atoms. In some embodiments, R and R¹⁰ are each hydrogen and R⁷ and R⁹ together form a bridge containing one —CH₂—moiety in addition to the two bridgehead atoms. In some embodiments, R⁸ and R¹⁰ are each hydrogen and R⁷ and R⁹ together form a bridge containing two —CH₂—moieties in addition to the two bridgehead atoms.

In some embodiments, R⁸ and R¹⁰ are each hydrogen.

In some embodiments, R⁸ and R¹⁰ are each hydrogen; R⁷ is methyl; and R⁹ is methyl. In some embodiments, R⁸ and R¹⁰ are each hydrogen; R⁷ is ethyl; and R⁹ is ethyl. In some embodiments, R⁸ and R¹⁰ are each hydrogen; R⁷ is methyl; and R⁹ is ethyl. In some embodiments, R⁸ and R¹⁰ are each hydrogen; R⁷ is ethyl, and R⁹ is methyl. In some embodiments, R⁸ and R¹⁰ are each hydrogen; R⁷ is methoxymethyl, and R⁹ is methyl, ethyl or methoxymethyl.

### The Definitions of L¹

In some embodiments, L¹ is a direct bond, —O—, —N(R^{L})—, -alkylene- or —C(O)—, wherein R^{L} is hydrogen or alkyl and wherein said -alkylene- is unsubstituted or substituted with deuterium, halogen, alkoxy, alkynyl or heterocyclyl. In some embodiments, L¹ is a direct bond, —O—, —N(R^{L})—, -alkylene- or —C(O)—, wherein R^{L} is hydrogen or alkyl. In some embodiments, L¹ is C₁₋₄alkylene, preferably C₁₋₂alkylene. In some embodiments, L¹ is a direct bond, —CH₂—, —CH(CH₃)—, —CH(CD₃)-, —CH(CH₂CH₃)—, —CH(CHF₂)—, —CH(prop-1-ynyl)-, —N(H)—, —N(CH₃)—, —O—, —CH(C(O)—NHCH₂CH₂OCH₃)—or —C(CH₃)₂—. In some embodiments, L¹ is —CH₂—, —CH(CH₃)—or —CH(CD₃)-. In some embodiments, L¹ is —CH(CH₃)—or —CH(CD₃)-.

The Definition of X² and X³

In some embodiments, X² and X³ are independently N or CH. In some embodiments, X² is N, and X³ is N. In some embodiments, X² is N, and X³ is CH. In some embodiments, X² is CH, and X³ is N. In some embodiments, X² is CH, and X³ is CH.

### The Definitions of Cy¹

In some embodiments, Cy¹ is aryl, heterocyclyl, heterocyclyl, heteroaryl, or cycloalkyl, each of which is unsubstituted or substituted with one, two, three, or four substituents R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl.

In some embodiments, Cy¹ is unsubstituted or substituted cycloalkyl, unsubstituted or substituted aryl, unsubstituted or substituted heterocyclyl, or unsubstituted or substituted heteroaryl. In some embodiments, Cy¹ is optionally substituted with one, two or three substituents R^{3a}, wherein R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropyl, isopropoxy, difluoromethoxy, cyclopropyl, 2,2-difluorocyclopropyl, 1-fluorocyclopropyl, 2-fluorocyclopropyl, cyclobutyl, 1-hydroxyethyl, 2-hydroxyethyl, ethyl, 1,1-difluoroethyl, cyano, dimethoxy, dichloro, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, 1-hydroxyazetidin-3-yl, 2-cyanoethyl, 2-cyanopropan-2-yl, hydroxymethyl, 3-hydroxypropyl, 2-hydroxypropan-2-yl, or 2-fluoropropan-2-yl.

In some embodiments, Cy¹ is aryl, which is unsubstituted or substituted with one, two or three substituents R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl.

In some embodiments, Cy¹ is aryl, which is unsubstituted or substituted with one, two or three substituents R^{3a}, wherein R^{3a} is selected from alkoxy, halogen-substituted alkoxy, alkoxyalkyl-, alkyl, alkenyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, heterocyclylalkyl- or heterocyclyl, said cycloalkyl or heterocyclyl is unsubstituted or substituted with alkoxy, alkyl, halogen, or hydroxy, wherein R^{3b} and R^{3c} are each independently hydrogen or alkyl.

In some embodiments, Cy¹ is phenyl, which is substituted with one or two R^{3a}, wherein R^{3a} is fluoro, chloro, bromo; methyl, difluoromethyl, trifluoromethyl, 1,1-difluoroethyl, methoxymethyl, 1-methoxyethyl, ethyl, propyl, isopropyl, tert-butyl, 2-hydroxypropan-2-yl, 1-hydroxy-2-methylpropan-2-yl, 2-cyanopropan-2-yl, morpholinomethyl; prop-1-en-2-yl; cyclopropyl, 1-methylcyclopropyl; methoxy, difluoromethoxy, trifluoromethoxy, 1,1-difluoroethoxy, isopropoxy; oxetan-3-yl, morpholino, 2-oxa-6-azaspiro[3.3]heptan-6-yl; or methylcarbamoyl.

In some embodiments, Cy¹ is an aryl of from 6 to 14 carbon atoms having a single ring or multiple condensed rings which is unsubstituted or substituted with one, two or three R^{3a}. In some embodiments, said aromatic carbocyclic group is phenyl, naphthyl or anthryl, indanyl, indenyl, dihydroindenyl or tetrahydronaphthyl, which is unsubstituted or substituted with one, two or three R^{3a}.

In some embodiments, Cy¹ is phenyl. In some embodiments, Cy¹ is phenyl, which is substituted with one R^{3a} as disclosed herein at position 4 and optionally substituted with R^{3a} on the other position.

In some embodiments, Cy¹ is naphthalenyl. In some embodiments, Cy¹ is naphthalen-1-yl, naphthalen-2-yl, naphthalen-3-yl, naphthalen-4-yl.

In some embodiments, Cy¹ is a monocyclic 5- to 9-membered heterocyclyl or a bicyclic 7- to 10-membered heterocyclyl which is unsubstituted or substituted with one, two, three or four R^{3a} (provided that the valency theory has been met), wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, oxo or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably, R^{3a} is selected from alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl. In some embodiments, said monocyclic 5- to 9-membered heterocyclyl is tetrahydrofuranyl, tetrahydropyranyl, 1,4-dioxanyl, piperidinyl, piperazinyl, or dihydropyridinyl, each of which is unsubstituted or substituted with one, two or three R^{3a} as disclosed herein. In some embodiments, Cy¹ is tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-1-yl, piperidin-2-yl, piperidin-3-yl, piperidin-4-yl, piperazin-1-yl, piperazin-2-yl, piperazin-3-yl, piperazin-4-yl, 1,2-dihydropyridin-3-yl, 1,2-dihydropyridin-4-yl, 1,2-dihydropyridin-5-yl, or 1,2-dihydropyridin-6-yl.

In some embodiments, Cy¹ is piperidinyl (e.g., piperidin-1-yl) or piperazinyl (e.g., piperazin-4-yl), which is substituted with one R^{3a} as disclosed herein at position 4 and optionally substituted with R^{3a} on the other position.

In some embodiments, Cy¹ is a bicyclic 7- to 10-membered heterocyclyl which is unsubstituted or substituted with one, two, three or four R^{3a} (provided that the valency theory has been met). In some embodiments, Cy¹ is dihydrobenzodioxinyl, benzodioxolyl, chromanyl, or chromenyl, which is unsubstituted or substituted with one, two, three or three R^{3a}, wherein R^{3a} is selected from alkoxy, alkyl, halogen, and oxo. In some embodiments, Cy¹ is 2,3-dihydrobenzo[b][1,4]dioxin-5-yl, 2,3-dihydrobenzo[b][1,4]dioxin-6-yl, benzo[d][1,3]dioxol-4-yl, benzo[d][1,3]dioxol-5-yl, chroman-2-yl, chroman-3-yl, chroman-4-yl, chroman-5-yl, chroman-6-yl, chroman-7-yl, chromen-2-yl, chromen-3-yl, chromen-4-yl, chromen-5-yl, chromen-6-yl, chromen-7-yl, each of which is unsubstituted or substituted with one, two, three or three R^{3a}, wherein R^{3a} is selected from alkoxy, alkyl, halogen, and oxo. In some embodiments, R^{3a} is methyl, ethyl, propyl, isopropyl, fluoro, chloro, bromo or oxo. In some embodiments, two R^{3a} connect to the same carbon and together form a spirocyclic ring, e.g.,

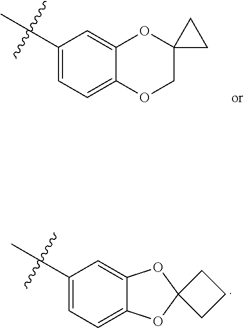 [SMILES: *c1ccc2c(c1)OC1(CC1)CO2 ; *c1ccc2c(c1)OC1(CCC1)O2]

In some embodiments, Cy¹ is a monocyclic 5- to 9-membered heteroaryl or a bicyclic 7- to 10-membered heteroaryl which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c}), N(R^{3b}R^{3c})—C(O)—, N(R^{3b}R^{3c}), R^{3b}—O—C(O)—, oxo, phenyl or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably, R^{3a} is selected from deuterium, alkoxy, halogen-substituted alkoxy, alkoxyalkyl-, alkyl, halogen-substituted alkyl, halogen, oxo, hydroxyalkyl-, cyano, cyano-substituted alkyl, N(R^{3b}R^{3c})—C(O)—, N(R^{3b}R^{3c})—, R^{3b}—O—C(O)—, oxo, phenyl or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, difluoromethyl, trifluoromethyl, trifluoromethoxy, methoxy, methoxymethyl, methylsulfonyl, ethoxy, isopropoxy, isopropyl, tert-butyl, cyclopropyl, 1-hydroxyethyl, ethyl, 1,1-difluoroethyl, cyano, cyclopropyl-, acetamido, 1-methoxyethyl, cyanomethyl, carbamoyl, methoxycarbonyl, methylcarbamoyl, dimethylcarbamoyl, (difluoromethoxy)methyl, amino, isopropylamino, (difluoromethoxy)ethyl, azetidin-1-yl, 2-methoxypropan-2-yl, 1-methoxycyclopropyl, oxetan-3-yl, 1-methylazetidin-3-yl, 1-hydroxyazetidin-3-yl, phenyl or oxo.

In some embodiments, said monocyclic 5- to 9-membered heteroaryl is pyrazolyl, imidazolyl, thiazolyl, pyridinyl, pyrimidinyl, pyrazinyl or pyridazinyl, each of which is unsubstituted or substituted with one, two or three R^{3a} as disclosed herein. In some embodiments, said monocyclic 5- to 9-membered heteroaryl is 1H-pyrazol-1-yl, 1H-pyrazol-3-yl, 1H-pyrazol-4-yl, thiazol-4-yl, thiazol-5-yl, 1H-pyrazol-5-yl, 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, pyrazin-2-yl, or pyridazin-4-yl, each of which is unsubstituted or substituted with one, two or three R^{3a} as disclosed herein. In some embodiments, R^{3a} is selected from deuterium, fluoro, bromo, chloro, methyl, difluoromethyl, trifluoromethyl, trifluoromethoxy, methoxy, ethoxy, isopropoxy, isopropyl, tert-butyl, carbamoyl, methylcarbamoyl, dimethylcarbamoyl, isopropylamino, azetidin-1-yl, phenyl or oxo.

In some embodiments, said bicyclic 7- to 10-membered heteroaryl is indolyl, triazolopyridinyl, imidazopyridinyl, benzooxazolyl, benzo[d]thiazolyl, quinolinyl, isoquinolinyl, naphthyridinyl, dioxinopyridineyl, quinoxalinyl, benzo[d]imidazolyl, benzofuranyl, benzoxazinyl, imidazo[4,5-b]pyridinyl, thiazolo[5,4-b]pyridinyl, thiazolo[4,5-b]pyridinyl, thieno[2,3-b]pyridinyl, dihydrodioxinopyridinyl, dioxinopyridinyl, dihydro-2H-pyranopyridinyl, 2H-pyranopyridinyl, dihydrofuropyridinyl, furopyridinyl, benzo[d]thiazolyl, isoindolinyl or thieno[3,2-b]pyridinyl, each of which is unsubstituted or substituted with one, two or three R^{3a} as disclosed herein. In some embodiments, said bicyclic 7- to 10-membered heteroaryl is quinoxalin-6-yl, quinoxalin-2-yl, isoquinolin-3-yl, isoquinolin-7-yl, quinolin-6-yl, 1H-benzo[d]imidazol-5-yl, benzofuran-6-yl, benzofuran-5-yl, benzo[e][1,3]oxazin-6-yl, 2,3-dihydro-[1,4]dioxino[2,3-b]pyridin-6-yl, 2,3-dihydro-[1,4]dioxino[2,3-b]pyridin-7-yl, 3,4-dihydro-2H-pyrano[3,2-b]pyridin-6-yl, 3,4-dihydro-2H-pyrano[3,2-b]pyridin-8-yl, 2,3-dihydrofuro[2,3-b]pyridin-6-yl, 2,3-dihydrofuro[3,2-b]pyridin-5-yl, benzo[d]thiazol-5-yl, benzo[d]thiazol-6-yl, isoindolin-5-yl, 1H-benzo[d]imidazol-2-yl, 1H-benzo[d]imidazol-4-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-7-yl, quinolin-2-yl, quinolin-3-yl, quinolin-4-yl, quinolin-5-yl, quinolin-7-yl, isoquinolin-1-yl, isoquinolin-4-yl, isoquinolin-5-yl, isoquinolin-6-yl, isoquinolin-8-yl, quinoxalin-3-yl, quinoxalin-4-yl, quinoxalin-5-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, quinoxalin-6-yl-2,3-d2, 1H-indol-2-yl, 1-methyl-1H-benzo[d]imidazol-6-yl, 3H-imidazo[4,5-b]pyridin-2-yl, 4,5,6,7-tetrahydro-1H-benzo[d]imidazol-2-yl, thiazolo[5,4-b]pyridin-2-yl, thiazolo[5,4-b]pyridin-5-yl, thiazolo[5,4-b]pyridin-6-yl, thiazolo[5,4-b]pyridin-7-yl, thiazolo[4,5-b]pyridin-2-yl, thiazolo[4,5-b]pyridin-5-yl, thiazolo[4,5-b]pyridin-6-yl, thiazolo[4,5-b]pyridin-7-yl, thieno[2,3-b]pyridin-2-yl, thieno[2,3-b]pyridin-3-yl, thieno[2,3-b]pyridin-4-yl, thieno[2,3-b]pyridin-5-yl, thieno[2,3-b]pyridin-6-yl, thieno[3,2-b]pyridin-2-yl, thieno[3,2-b]pyridin-3-yl, thieno[3,2-b]pyridin-5-yl, thieno[3,2-b]pyridin-6-yl, thieno[3,2-b]pyridin-7-yl, each of which is unsubstituted or substituted with one, two or three R^{3a} as disclosed herein.

In some embodiments, Cy¹ is quinoxalinyl, e.g., quinoxalin-2-yl, quinoxalin-3-yl, quinoxalin-4-yl, quinoxalin-5-yl, quinoxalin-6-yl, quinoxalin-7-yl, quinoxalin-8-yl, preferably quinoxalin-6-yl, which is unsubstituted or substituted with one, two or three R^{3a}, wherein R^{3a} is selected from deuterium, alkoxy, alkyl, halogen, oxo, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably, R^{3a} is selected from deuterium, alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c}) cyano-substituted alkyl, N(R^{3b}R^{3c})—C(O)—, N(R^{3b}R^{3c})—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropoxy, trifluoromethoxy, difluoromethoxy, cyclopropyl, 1-hydroxyethyl, ethyl, 1,1-difluoroethyl, cyano, dimethoxy, dichloro, cyclopropyl-, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, or 1-hydroxyazetidin-3-yl.

In some embodiments, Cy¹ is quinoxalin-2-yl or quinoxalin-6-yl, which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2,2,2-trifluoroethyl, 2-fluoropropan-2-yl, methoxy, isopropoxy, difluoromethoxy, fluoro, chloro, oxo, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl. In some embodiments, Cy¹ is quinoxalin-6-yl, 3-methylquinoxalin-6-yl, 3-(trifluoromethyl)quinoxalin-6-yl, or quinoxalin-2-yl.

In some embodiments, Cy¹ is benzo[d]thiazol-5-yl, benzo[d]thiazol-6-yl, isoindolin-4-yl, indolin-5-yl, 1H-benzo[d]imidazol-5-yl, benzofuran-6-yl, benzofuran-5-yl, or benzo[e][1,3]oxazin-6-yl, isoquinolin-3-yl, isoquinolin-7-yl, quinolin-6-yl, 2,3-dihydro-[1,4]dioxino[2,3-b]pyridin-6-yl, 2,3-dihydro-[1,4]dioxino[2,3-b]pyridin-7-yl, 3-dihydro-[1,4]dioxino[2,3-c]pyridin-7-yl, 3,4-dihydro-2H-pyrano[3,2-b]pyridin-6-yl, 3,4-dihydro-2H-pyrano[3,2-b]pyridin-8-yl, 2,3-dihydrofuro[2,3-b]pyridin-6-yl, 2,3-dihydrofuro[3,2-b]pyridin-5-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2,2,2-trifluoroethyl, 2-fluoropropan-2-yl, methoxy, isopropoxy, difluoromethoxy, fluoro, chloro, bromo, oxo, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

In some embodiments, Cy¹ is
4-fluoro-2-methoxyphenyl, p-tolyl, 4-fluoro-2-(trifluoromethyl)phenyl, 2-(difluoromethoxy)-4-fluorophenyl, 4-fluoro-2-(trifluoromethoxy)phenyl, 4-fluoro-2-(methoxymethyl)phenyl, 4-fluoro-2-(1-methoxyethyl)phenyl, 1-(difluoromethoxy-4-fluorophenyl, 2-(difluoromethyl)-4-fluorophenyl, 2-(1,1-difluoroethoxy)-4-fluorophenyl, 3-(trifluoromethyl)phenyl, 3-isopropoxyphenyl, 3-(trifluoromethoxy)phenyl, 3-isopropylphenyl, 3-(tert-butyl)phenyl, 3-(2-hydroxypropan-2-yl)phenyl, 3-(difluoromethyl)-4-fluorophenyl, 4-fluoro-3-(trifluoromethyl)phenyl, 2-fluoro-5-(trifluoromethyl)phenyl, 4-methyl-3-(trifluoromethyl)phenyl, 4-methyl-2-(trifluoromethyl)phenyl, 3-cyclopropylphenyl, 3-(1-methylcyclopropyl)phenyl, 3-(oxetan-3-yl)phenyl, 4-fluoro-3-(methylcarbamoyl)phenyl, 3-(1-hydroxy-2-methylpropan-2-yl)phenyl, 3-(2-cyanopropan-2-yl)phenyl, 4-fluoro-3-(prop-1-en-2-yl)phenyl, 4-fluoro-3-(1-methylcyclopropyl)phenyl, 3-methyl-4-(trifluoromethyl)phenyl, 4-(morpholinomethyl)phenyl, 3-(2-oxa-6-azaspiro[3.3]heptan-6-yl)phenyl, 3-morpholinophenyl, 3-(morpholinomethyl)phenyl, 4-morpholinophenyl, 4-(trifluoromethyl)phenyl, 4-fluorophenyl, 3-methyl-2-(trifluoromethyl)phenyl, 3-(difluoromethyl)-5-fluorophenyl, 3-(1,1-difluoroethyl)-4-methoxyphenyl, m-tolyl, 3-methoxyphenyl, or 3-(difluoromethyl)phenyl; or
3,3-dimethyl-2,3-dihydro-1H-inden-5-yl; or
4,4-difluorochroman-6-yl, 2,2-dimethyl-4-oxochroman-7-yl, 3-methyl-4-oxo-4H-chromen-6-yl, 2,2-dimethylchroman-7-yl, or 2-methyl-4-oxo-4H-chromen-7-yl; or
2,3-dihydrobenzo[b][1,4]dioxin-6-yl, 3,3-dimethyl-2,3-dihydrobenzo[b][1,4]dioxin-6-yl, 8-fluoro-3,3-dimethyl-2,3-dihydrobenzo[b][1,4]dioxin-6-yl, 7-fluoro-3,3-dimethyl-2,3-dihydrobenzo[b][1,4]dioxin-6-yl, 5-fluoro-3,3-dimethyl-2,3-dihydrobenzo[b][1,4]dioxin-6-yl, 8-chloro-3,3-dimethyl-2,3-dihydrobenzo[b][1,4]dioxin-6-yl, 2,2,3,3-tetrafluoro-2,3-dihydrobenzo[b][1,4]dioxin-6-yl, 3,3-difluoro-2,3-dihydrobenzo[b][1,4]dioxin-6-yl, 7-fluoro-2,2-dimethyl-2,3-dihydrobenzo[b][1,4]dioxin-6-yl, or 7-chloro-3,3-dimethyl-2,3-dihydrobenzo[b][1,4]dioxin-6-yl; or
2,2-dimethylbenzo[d][1,3]dioxol-5-yl, 2,2-difluorobenzo[d][1,3]dioxol-5-yl, 2-ethyl-2-methylbenzo[d][1,3]dioxol-5-yl, spiro[benzo[d][1,3]dioxole-2,1′-cyclobutan]-5-yl, 6-chloro-2,2-dimethylbenzo[d][1,3]dioxol-5-yl, 7-chloro-2,2-dimethylbenzo[d][1,3]dioxol-5-yl, 4-chloro-2,2-dimethylbenzo[d][1,3]dioxol-5-yl, 7-fluoro-2,2-dimethylbenzo[d][1,3]dioxol-5-yl, 4-fluoro-2,2-dimethylbenzo[d][1,3]dioxol-5-yl, 4,6-difluoro-2,2-dimethylbenzo[d][1,3]dioxol-5-yl, 2,2,7-trimethylbenzo[d][1,3]dioxol-5-yl, 7-fluoro-2,2-dimethylbenzo[d][1,3]dioxol-4-yl, 2,2-dimethylbenzo[d][1,3]dioxol-4-yl, 2,2-difluorobenzo[d][1,3]dioxol-4-yl, 4-fluoro-2,2-dimethylbenzo[d][1,3]dioxol-5-yl, or 2,2-dimethylbenzo[d][1,3]dioxol-5-yl; or
2-(trifluoromethyl)pyridin-3-yl, 6-isopropoxypyridin-2-yl, 6-(trifluoromethyl)pyridin-2-yl, 2-(trifluoromethyl)pyridin-4-yl, 4-(trifluoromethyl)pyridin-2-yl, 2-(azetidin-1-yl)pyridin-4-yl, 2-(isopropylamino)pyridin-4-yl, 6-isopropylpyridin-2-yl, 2-isopropoxypyridin-4-yl, 6-(trifluoromethoxy)pyridin-2-yl, 2-(tert-butyl)pyridin-4-yl, 2-(methylcarbamoyl)pyridin-4-yl, 2-(2,2-dimethylcarbamoyl)pyridin-4-yl, 5,6-dimethylpyridin-2-yl, 2-phenylpyridin-4-yl, 5-(difluoromethyl)pyridin-2-yl, 5-methylpyridin-2-yl, 5-(trifluoromethyl)pyridin-2-yl, 2-methoxypyridin-4-yl, 1-methyl-6-oxo-1,6-dihydropyridin-3-yl, 1-methyl-2-oxo-1,2-dihydropyridin-4-yl, 2-(trifluoromethyl)thiazol-4-yl, or 1-methyl-5-(trifluoromethyl)-1H-pyrazol-4-yl; or
quinoxalin-6-yl, 3-methylquinoxalin-6-yl, 3-(trifluoromethyl)quinoxalin-6-yl, or quinoxalin-2-yl; or
isoquinolin-3-yl, 2-methyl-1-oxo-1,2-dihydroisoquinolin-7-yl, 2-methyl-1-oxo-1,2,3,4-tetrahydroisoquinolin-7-yl, 1,2-dimethyl-4-oxo-1,4-dihydroquinolin-7-yl, 1,3-dimethyl-4-oxo-1,4-dihydroquinolin-6-yl, or 2-methylquinolin-7-yl; or
2-methyl-1H-benzo[d]imidazol-5-yl, 2,2-dimethyl-2,3-dihydrobenzofuran-6-yl, 2,2-dimethyl-3-oxo-2,3-dihydrobenzofuran-6-yl, 3,3-dimethyl-2,3-dihydrobenzofuran-5-yl, or 3-methyl-4-oxo-3,4-dihydro-2H-benzo[e][1,3]oxazin-6-yl;
2,3-dihydro-[1,4]dioxino[2,3-b]pyridin-6-yl, 3,3-dimethyl-2,3-dihydro-[1,4]dioxino[2,3-b]pyridin-6-yl, 4,4-difluoro-3,4-dihydro-2H-pyrano[3,2-b]pyridin-6-yl, 4,4-difluoro-3,4-dihydro-2H-pyrano[3,2-b]pyridin-8-yl,
7-chloro-3,3-dimethyl-2,3-dihydro-[1,4]dioxino[2,3-b]pyridin-6-yl, 7-fluoro-3,3-dimethyl-2,3-dihydro-[1,4]dioxino[2,3-b]pyridin-6-yl, 8-fluoro-3,3-dimethyl-2,3-dihydro-[1,4]dioxino[2,3-b]pyridin-6-yl, 2,2-dimethyl-2,3-dihydrofuro[2,3-b]pyridin-6-yl, 3,3-dimethyl-2,3-dihydrofuro[3,2-b]pyridin-5-yl, 3,3-dimethyl-2,3-dihydrofuro[3,2-b]pyridine-5-yl, 2,2-dimethyl-2,3-dihydro-[1,4]dioxino[2,3-c]pyridin-7-yl, 2,2-dimethyl-2,3-dihydro-[1,4]dioxino[2,3-b]pyridin-7-yl, 3-methyl-2,3-dihydro-[1,4]dioxino[2,3-b]pyridin-6-yl, or 3,3-dimethyl-2,3-dihydro-[1,4]dioxino[2,3-b]pyridin-6-yl; or
2-methylbenzo[d]thiazol-5-yl, 2-methylbenzo[d]thiazol-6-yl, 2-ethylbenzo[d]thiazol-5-yl, 2-isopropylbenzo[d]thiazol-5-yl, or 2-(trifluoromethyl)thiazol-5-yl; or
2-methyl-1,3-dioxoisoindolin-5-yl, 2-isopropyl-1,3-dioxoisoindolin-5-yl, 1-oxo-2-(2,2,2-trifluoroethyl)isoindolin-5-yl, 2,3,3-trimethyl-1-oxoisoindolin-5-yl, 2-methyl-3-oxoisoindolin-5-yl, or 2-methyl-1-oxoisoindolin-5-yl.

In some embodiments, the moiety m is

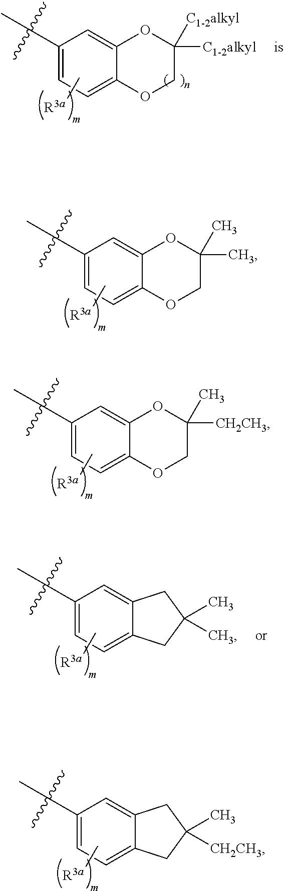

wherein m is 0, 1, 2 or 3 and R^{3a} is alkoxy, alkyl, halogen, and oxo.

In some embodiments, the moiety

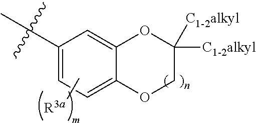

is 2,2-dimethylbenzo[d][1,3]dioxol-5-yl or 3,3-dimethyl-2,3-dihydrobenzo[b][1,4]dioxin-6-yl, wherein m is 0, 1, 2 or 3 and R^{3a} is alkoxy, alkyl, halogen, and oxo. In some embodiments, the moiety

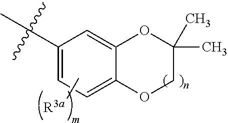

is 2,2-dimethylbenzo[d][1,3]dioxol-5-yl, 2,2-difluorobenzo[d][1,3]dioxol-5-yl, 2-ethyl-2-methylbenzo[d][1,3]dioxol-5-yl, spiro[benzo[d][1,3]dioxole-2,1′-cyclobutan]-5-yl, 6-chloro-2,2-dimethylbenzo[d][1,3]dioxol-5-yl, 7-chloro-2,2-dimethylbenzo[d][1,3]dioxol-5-yl, 4-chloro-2,2-dimethylbenzo[d][1,3]dioxol-5-yl, 7-fluoro-2,2-dimethylbenzo[d][1,3]dioxol-5-yl, 4-fluoro-2,2-dimethylbenzo[d][1,3]dioxol-5-yl, 4,6-difluoro-2,2-dimethylbenzo[d][1,3]dioxol-5-yl, 2,2,7-trimethylbenzo[d][1,3]dioxol-5-yl, 2,2-difluorobenzo[d][1,3]dioxol-4-yl, 4-fluoro-2,2-dimethylbenzo[d][1,3]dioxol-5-yl, 2,2-dimethylbenzo[d][1,3]dioxol-5-yl; 2,3-dihydrobenzo[b][1,4]dioxin-6-yl, 3,3-dimethyl-2,3-dihydrobenzo[b][1,4]dioxin-6-yl, 8-fluoro-3,3-dimethyl-2,3-dihydrobenzo[b][1,4]dioxin-6-yl, 7-fluoro-3,3-dimethyl-2,3-dihydrobenzo[b][1,4]dioxin-6-yl, 5-fluoro-3,3-dimethyl-2,3-dihydrobenzo[b][1,4]dioxin-6-yl, 8-chloro-3,3-dimethyl-2,3-dihydrobenzo[b][1,4]dioxin-6-yl, 2,2,3,3-tetrafluoro-2,3-dihydrobenzo[b][1,4]dioxin-6-yl, 3,3-difluoro-2,3-dihydrobenzo[b][1,4]dioxin-6-yl, 7-fluoro-2,2-dimethyl-2,3-dihydrobenzo[b][1,4]dioxin-6-yl, or 7-chloro-3,3-dimethyl-2,3-dihydrobenzo[b][1,4]dioxin-6-yl.

### General Synthetic Schemes

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from room temperature to the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica flash column chromatography.

Chiral analytic HPLC was used for the retention time analysis of different chiral examples, the conditions were divided into the methods as below according to the column, mobile phase, solvent ration used. Preparation of homochiral examples may be carried out by techniques known to one skilled in the art. The absolute stereochemistry was not assigned at the newly formed carbon-nitrogen bond.

Abbreviations:

| | |
|---|---|
| Ac | Acetyl |
| AcOH | Acetic acid |
| AcONa | Sodium acetate |
| AIBN | (E)-2,2′-(diazene-1,2-diyl)bis(2-methylpropanenitrile) |
| Aq | Aqueous |
| BINAP | 2,2′-bis(diphenylphosphaneyl)-1,1′-binaphthalene |
| Brine | Saturated aqueous sodium chloride solution |
| Bn | Benzyl |
| Boc | Tert-butyloxycarbonyl |
| (Boc)₂O | Di-tert-butyl dicarbonate |
| BOP | Benzotriazol-1-yloxytris-(dimethylamino)phosphonium |
| | hexafluorophosphate |
| DBU | 1,8-Diazabicyclo[5.4.0]undec-7-ene |
| DMF | N,N-Dimethylformamide |
| DBU | 1,8-diazabicyclo[5.4.0]undec-7-ene |
| DCE | 1,2-dichloroethane |
| DCM | Dichloromethane |
| DIBAL-H | N,N-diisobutylaluminum hydride |
| DIPEA | N,N-diisopropylethylamine |
| DMAc | N,N-dimethylacetamide |
| DMAP | 4-N,N-dimethylaminopyridine |
| DMF | N,N-dimethylformamide |
| DMSO | Dimethyl sulfoxide |
| EtOAc | Ethyl acetate |
| Eq | Equivalent |
| EtI | Iodoethane |
| EtOH | Ethanol |
| Et₂Zn | Diethylzinc |
| Et₃N | Triethylamine |
| g | Grams |
| HATU | O-(7-Azabenzotriazol-1-yl)-N,N,N′,N′-tetramethyluronium |
| | hexafluorophosphate |
| HCl | Hydrochloric acid |
| HPLC | High-performance liquid flash column chromatography |
| IPA | Isopropyl alcohol |
| LiHMDS | Lithium bis(trimethylsilyl)amide |
| LDA | Lithium diisopropylamide |
| mg | Milligrams |
| mL | Milliliters |
| mmol | Millimole |
| MeCN | Acetonitrile |
| MeI | Iodomethane |
| MeOH | Methanol |
| Min | Minutes |
| MeMgBr | Methylmagnesium bromide |
| MS | Mass spectrum |
| MsCl | Methanesulfonyl chloride |
| n-Bu₃SnH | Tributylstannane |
| n-BuLi | Butyllithium |
| n-BuOH | Butan-1-ol |
| n-Bu₄NF | Tetrabutylammonium fluoride |
| NMR | Nuclear magnetic resonance |
| NCS | N-Chlorosuccinimide |
| NBS | N-Bromosuccinimide |
| Pd/C | Palladium on carbon |
| Pd(dppf)Cl₂ | [1,1′-Bis(diphenylphosphino)ferrocene]dichloropalladium |
| Pd(OAc)₂ | Palladium diacetate |
| Pd(PPh₃)₂Cl₂ | Dichlorobis(triphenylphosphine)palladium |
| Pd(PPh₃)₄ | Tetrakis(triphenylphosphine)palladium |
| Pd₂(dba)₃ | Tris(dibenzylideneacetone)dipalladium |
| PE | Petroleum ether |
| PPh₃ | Triphenylphosphane |
| RT | room temperature |
| Selectfluor | 1-(chloromethyl)-4-fluoro-1,4-diazabicyclo[2.2.2]octane-1,4-diium |
| | tetrafluoroborate |
| TBAF | Tetrabutylammonium fluoride |
| TBSCl | tert-Butyldimethylsilyl chloride |
| tBuXPhos | Bis(1,1-dimethylethyl)[2′,4′,6′-tris(1-methylethyl)[1,1′-biphenyl]-2- |
| | yl]phosphine |
| tBuXPhos Pd G3 | Methanesulfonato(2-di-t-butylphosphino-2′,4′,6′-tri-i-propyl-1,1′- |
| | biphenyl)(2′-amino-1,1′-biphenyl-2-yl)palladium(II) |
| t-BuOK | Potassium 2-methylpropan-2-olate |
| TEA | Triethanolamine |
| TFA | Trifluoroacetic acid |
| THF | Tetrahydrofuran |
| THP | Tetrahydropyran3,4-dihydro-2H-pyran |
| TLC | Thin layer flash column chromatography |
| TMSCN | Trimethylsilanecarbonitrile |
| TrtCl | Triphenylmethyl Chloride |
| XPhos | 2-Dicyclohexylphosphino-2′,4′,6′-triisopropylbiphenyl |

### General Synthetic Schemes

Compounds disclosed herein, including salts thereof, can be prepared using known organic synthesis techniques and can be synthesized according to any of numerous possible synthetic routes.

The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from room temperature to the solvent's boiling temperature. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica flash column chromatography.

Chiral analytic HPLC was used for the retention time analysis of different chiral examples, the conditions were divided into the methods as below according to the column, mobile phase, solvent ration used. Preparation of homochiral examples may be carried out by techniques known to one skilled in the art. The absolute stereochemistry was not assigned at the newly formed carbon-nitrogen bond.

The compounds disclosed herein can be prepared by following Scheme I to V.

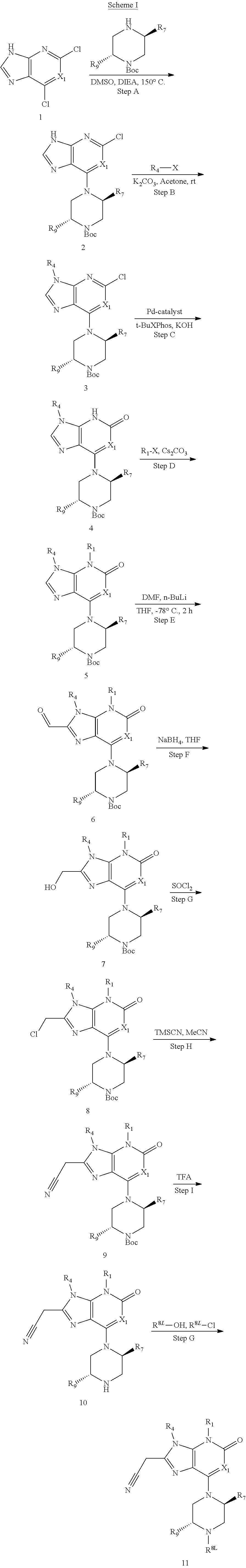

wherein the substitutions from R¹ to R⁹ and R^{8L} (corresponding to L¹-Cy¹) are as defined as in Formula (I).

In scheme I, a commercially available Compound 1 (X₁ is CH or N) is reacted with the appropriate chiral secondary amine by nucleophilic aromatic substitution reaction to give Compound 2. Compound 2 is reacted with appropriate R₄—X under basic condition (such as K₂CO₃, Cs₂CO₃) to give Compound 3. Compound 3 can be used to produce Compound 4 by normally Pd-catalyzed C—O coupling reaction with appropriate Pd catalyst and ligand under a basic condition (such as KOH). Compound 4 is reacted with appropriate R₁—X under a basic condition (such as K₂CO₃, Cs₂CO₃) to give Compound 5. Compound 5 is reacted with formylation reagent (such as DMF) to give Compound 6 under a basic condition (such as n-BuLi or LDA). Reduction of Compound 6 to give Compound 7 as a alcohol using reducing agent (such as NaBH₄). Compound 7 wherein contains a hydroxyl group may be chlorinated by treatment with a chlorination agent such as (SOCl₂) to give Compound 8. Compound 8 as a benzyl halide may be converted into corresponding Compound 9 by treatment with a cyanation agent (such as TMSCN) under a basic condition (such as Cs₂CO₃ or n-Bu₄NF). Compound 9 is deprotected using an acid condition (such as TFA or 4M solution of HCl in 1,4-dioxane) to give Compound 10. Tertiary amines Compound 11 is prepared by N-alkylation of secondary amines compound 10 by treatment with reductive alkylation with aldehydes or ketones, the most frequently used procedures via a phosphonium salt mediated alkylation of amines with corresponding alcohols (Florencio Zaragoza and Henrik Stephensen, J. Org. Chem. 2001, 66, 2518-2521).

Compound 7 disclosed herein can also be prepared by the following Scheme II.

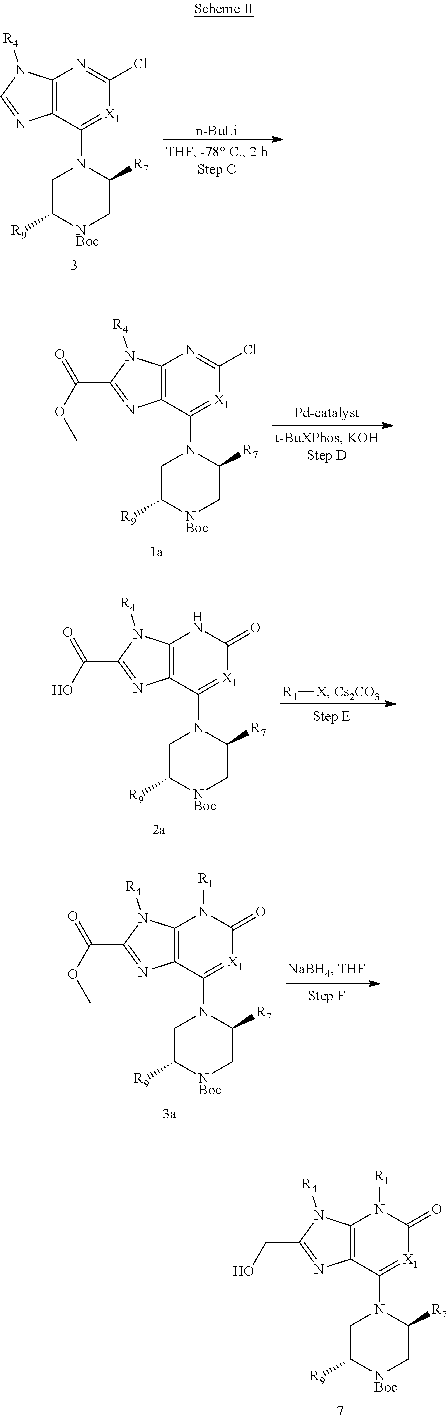

In Scheme II, Compound 3 may be converted into corresponding Compound 1a by treatment with a strong base (such as n-BuLi) and methyl chloroformate. Compound 1a wherein contains a chlorine atom may be converted into a corresponding compound containing a hydroxyl group by normally Pd-catalyzed C—O coupling reaction with appropriate Pd catalyst and ligand under a basic condition (such as KOH), meanwhile, the ester group of Compound 1a is hydrolyzed to carboxylic acid of Compound 2a. Compound 2a is reacted with appropriate R₁—X under a basic condition (such as K₂CO₃, Cs₂CO₃) to give Compound 3a. Reduction of Compound 3a to give Compound 7 as a alcohol using reducing agent (such as NaBH₄).

Compound 7 disclosed herein can also be prepared by the following Scheme III.

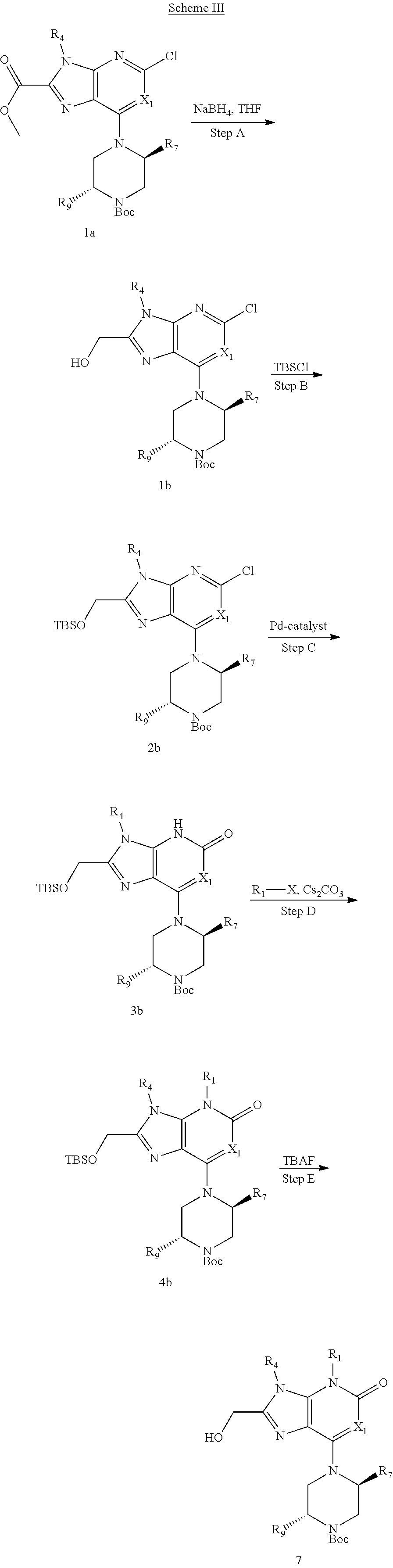

In Scheme III, the Reduction of Compound 1a to give Compound 1b as a alcohol using a reducing agent (such as NaBH₄). Compound 1b may be converted into corresponding Compound 2b by treatment with an organic base (such as imidazole) and TBSCl. Compound 2b wherein contains a chlorine atom may be converted into corresponding Compound 3b containing a hydroxyl group by normally Pd-catalyzed C—O coupling reaction with appropriate Pd catalyst and ligand under a basic condition (such as KOH). Compound 3b is reacted with appropriate R₁—X under basic condition (such as K₂CO₃, Cs₂CO₃) to give Compound 4b. Compound 4b is deprotected using a reagent containing fluorine (such as tetrabutylammonium fluoride) to give Compound 7.

The compound of Formula 12c disclosed herein can be prepared by following Scheme IV.

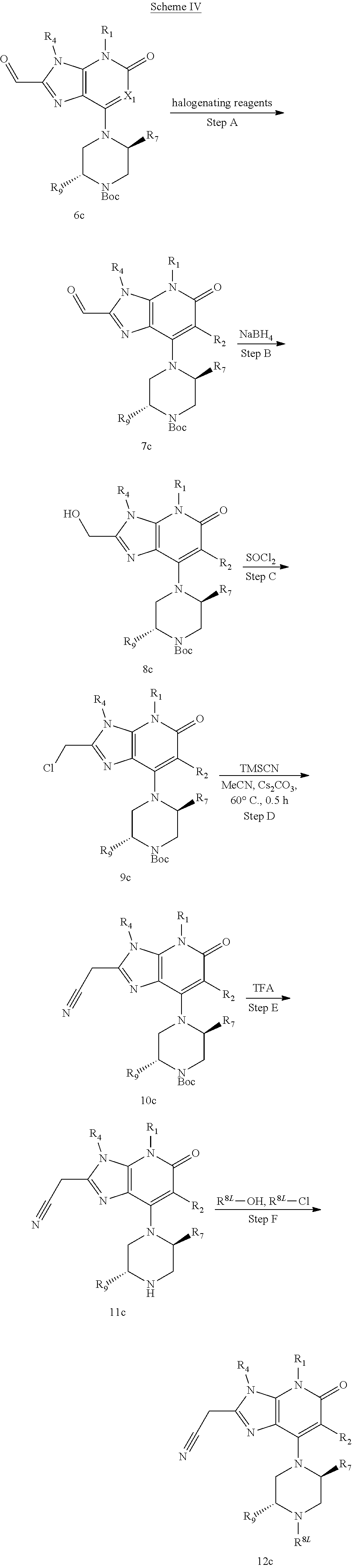

The halogenation of Compound 6c with electrophilic halogenating reagents (such as NBS, NCS, or Selectfluor) to afford a Compound 7c. Reduction of Compound 7c to give Compound 8c as a alcohol using a reductive agent (such as NaBH₄). Compound 8c wherein contains a hydroxyl group may be chlorinated by treatment with a chlorination agent such as (SOCl₂ or MsCl) to give Compound 9c. Compound 9c as a benzyl halide may be converted into corresponding Compound 10c by treatment with a cyanation agent (such as TMSCN) under a basic condition (such as Cs₂CO₃ or n-Bu₄NF). Compound 10c is deprotected using an acid condition (such as TFA or 4M solution of HCl in 1,4-dioxane) to give Compound 11c. Tertiary amines Compound 12c is prepared by N-alkylation of secondary amines compound 11c by treatment with reductive alkylation with aldehydes or ketones, the most frequently used procedures via a phosphonium salt mediated alkylation of amines with corresponding alcohols (Florencio Zaragoza and Henrik Stephensen, J. Org. Chem. 2001, 66, 2518-2521).

The compound of Formula 11d disclosed herein can also be prepared by following Scheme V.

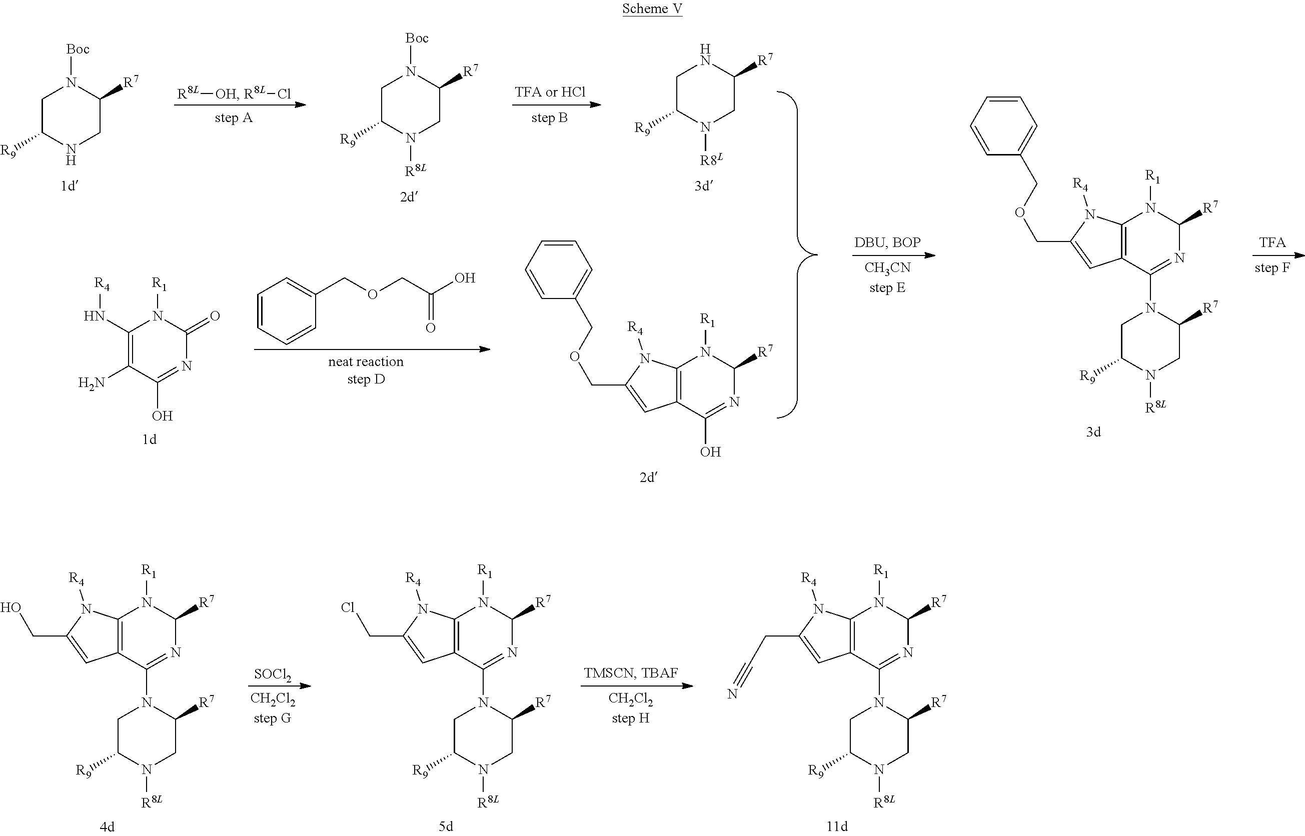

In scheme V, Compound 2d′ is prepared by N-alkylation of secondary amines Compound 1d′ by treatment with alkylating agents (such as alkyl halides or sulfonates) or reductive alkylation with aldehydes or ketones, the most frequently used procedures via a phosphonium salt mediated alkylation of amines with corresponding alcohols. Compound 2d′ is deprotected using acid conditions (such as TFA or 4M solution of HCl in 1,4-dioxane) to give Compound 3d′. Compound 1d was synthesized according to the method described in the literature (Shaker Youssef and Wolfgang Pfleiderer, J. Heterocyclic Chem. 35, 949-954, 1998). Compound 1d is reacted with the appropriate acid via cyclization reaction to give Compound 2d. Compound 2d is reacted with the appropriate chiral secondary amine (corresponding to Compound 3d′) via phosphonium-mediated S_{N}Ar reactions to give Compound 3d in the presence of phosphonium reagents (such as BOP) under a basic condition (such as DBU, DIPEA). Compound 3d is deprotected using an acid condition (such as TFA) to give Compound 4d. Compound 4d wherein contains a hydroxyl group may be chlorinated by treatment with a chlorination agent such as (SOCl₂) to give Compound 5d. Compound 5d as a benzyl halide may be converted into corresponding Compound 11d by treatment with a cyanation agent (such as TMSCN) under a basic condition (such as Cs₂CO₃ or n-Bu₄NF).

### Examples

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for flash column chromatography and silica gel (GF254) for thin-layer flash column chromatography (TLC) are commercially available from Tsingdao Haiyang Chemical Co., Ltd. or Yantai Chemical Co., Ltd. of China; all are eluted with petroleum ether (60-90° C.)/ethyl acetate (v/v), and visualized by iodine or the solution of molybdphosphoric acid in ethanol unless otherwise specified. All extraction solvents, unless otherwise specified, are dried over anhydrous Na₂SO₄. ¹H NMR spectra are recorded on Bruck-400 nuclear magnetic resonance spectrometer with TMS (tetramethylsilane) as the internal standard. LC/MS data are recorded by using Agilent1100 High Performance Liquid Flash column chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw®.

### Synthesis

Preparative HPLC Conditions (Method A)

| | |
|---|---|
| Column | Phenomenex Gemini NX-C18, 150 × 21.2 mm, 5 |
| | μm |
| Column Temp. | R.T. |
| Detection Wavelength | DAD, UV λ = 214/254 nm |
| Run Time | 17.0 min |
| Flow Rate | 20.0 mL/min |
| Mobile Phase A | 0.1% FA-H₂O (v/v) |
| Mobile Phase B | 0.1% FA-CH₃CN (v/v) |

Preparative HPLC Conditions (Method B)

| | |
|---|---|
| Column | Waters XSelect CSH C18, 150 × 19 mm, 5 μm |
| Column Temp. | R.T. |
| Detection Wavelength | DAD, UV λ = 214/254 nm |
| Run Time | 17.0 min |
| Flow Rate | 17 mL/min |
| Mobile Phase A | 0.03% NH₃•H₂O—H₂O (v/v) |
| Mobile Phase B | CH₃CN |

### Intermediate 1: tert-butyl (2S,5R)-2,5-diethylpiperazine-1-carboxylate

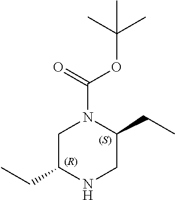

### Step A: methyl (R)-2-(benzylamino)butanoate

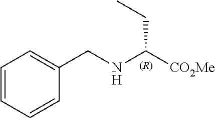

To a solution of methyl (R)-2-aminobutanoate (100.0 g, 0.85 mol) in CH₃CN (1000 mL) was added benzyl bromide (146.1 g, 0.85 mol) at 0° C. under N₂ atmosphere. The reaction was stirred at room temperature overnight and concentrated under reduced pressure. The resulting residue was dissolved in EtOAc (1000 mL) and washed with water (1000 mL×3). The organic layers were concentrated under reduced pressure. The resulting residue was purified by flash column chromatography (PE:EtOAc=10:1) to give the titled compound (106 g, 600%). MS: M/e 208 (M+1)⁺.

### Step B: methyl (R)-2-((S)—N-benzyl-2-((tert-butoxycarbonyl)amino)butanamido) butanoate

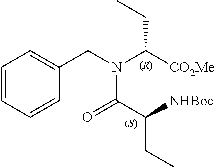

To a solution of methyl (R)-2-(benzylamino) butanoate (117.0 g, 0.56 mol), (S)-2-((tert-butoxycarbonyl)amino)butanoic acid (170.5 g, 0.84 mmol) and 4-Methylmorpholine (113.1 g, 1.12 mmol) in DCM (2000 mL) was added HATU (319.0 g, 0.84 mmol) at 0° C. The reaction was stirred at room temperature overnight and quenched by water and washed with water (1500 mL×2). The organic layers were concentrated under reduced pressure. The resulting residue was purified by flash column chromatography (PE:EtOAc=10:1) to give the titled compound (178 g, 80%). MS: Me 393 (M+1)+.

### Step C: methyl (R)-2-((S)-2-amino-N-benzylbutanamido)butanoate

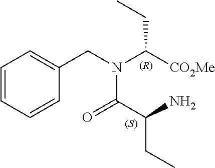

To a solution of methyl (R)-2-((S)—N-benzyl-2-((tert-butoxycarbonyl)amino)butanamido) butanoate (178 g, 0.45 mol) in 1,4-dioxane (100 mL) was added HCl (400 mL, 4 M in 1,4-dioxane) at room temperature. The resulting mixture was stirred at room temperature for another 2 hours and concentrated under vacuum to give the titled compound (200 g, crude). MS: M/e 293 (M+1)+.

### Step D: (3S,6R)-1-benzyl-3,6-diethylpiperazine-2,5-dione

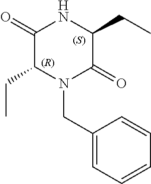

To a solution of methyl (R)-2-((S)-2-amino-N-benzylbutanamido)butanoate (200 g, crude) in EtOAc (1000 mL) was added aq. NaHCO₃ (300 mL) at room temperature. The reaction mixture was stirred at room temperature for another 2 hours. The organic layers were concentrated under reduced pressure. The resulting residue was triturated with MTBE to give the titled compound (61 g, 52% for 2 steps, ee: 97%). MS: M/e 261 (M+1)⁺.

### Step E: (2R,5S)-1-benzyl-2,5-diethylpiperazine

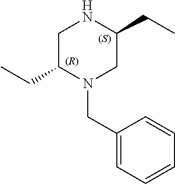

To a solution of LiAlH₄ (26.5 g, 0.69 mol) in THE (1000 mL) was added slowly (3S,6R)-1-benzyl-3,6-diethylpiperazine-2,5-dione (61.0 g, 0.23 mol) in THE (500 mL) at 0° C. The resulting mixture was stirred at room temperature for 2 hours, then stirred at 80° C. overnight. The reaction was quenched by water (27 mL) slowly at 0° C. Then, 1N aqueous NaOH solution (54 mL) and water (81 mL) was added sequentially. The resulting mixture was stirred for 2 hours. The white precipitates that formed was removed by filtration. The filter cake was washed with EtOAc (500 mL). The combined filtrates were evaporated. The resulting residue was dissolved in toluene. The solvent was removed under vacuum to dryness to afford the titled compound (51 g, 95%). MS: M/e 233 (M+1)⁺.

### Step F: tert-butyl (2S,5R)-4-benzyl-2,5-diethylpiperazine-1-carboxylate

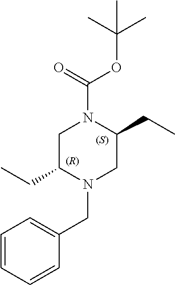

To a solution of (2R,5S)-1-benzyl-2,5-diethylpiperazine (46.4 g, 200 mmol) and Boc₂O (48 g, 220 mmol) in DCM (800 mL) was added Et₃N (30.3 g, 300 mmol). The reaction mixture was stirred at room temperature for 12 hours. The reaction was quenched with saturated NaCl (400 mL) at room temperature. The resulting mixture was extracted with DCM (800 mL×2). The combined organic layers were dried over Na₂SO₄ and concentrated under reduced pressure. The resulting residue was purified by flash column chromatography to give the titled compound (43.2 g, 65%). MS: M/e 333 (M+1)⁺.

### Step G: tert-butyl (2S,5R)-2,5-diethylpiperazine-1-carboxylate

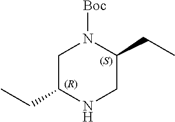

To a solution of tert-butyl (2S,5R)-4-benzyl-2,5-diethylpiperazine-1-carboxylate (43.2 g, 130 mmol) and Pd/C (6.6 g, 10% in water) in MeOH (240 mL) at room temperature was added AcOH (5 mL). The resulting mixture was degassed 3 times under H₂ atmosphere and stirred at room temperature under H₂ atmosphere for 12 hours. After filtration, the combined organic layers were concentrated under reduced pressure to give the crude product. The crude product was basified by Na₂CO₃ (4M) to pH ˜10 and extracted with EA (200 mL×3). The combined organic layers were washed with brine (20 mL×3), dried over Na₂SO₄ and concentrated under reduced pressure to give the titled compound (22 g, 70%). ¹H NMR (400 MHz, CDCl₃) δ 3.96-3.84 (m, 1H), 3.74 (d, J=13.2 Hz, 1H), 3.12-3.00 (m, 2H), 2.72-2.60 (m, 1H), 2.52 (dd, J=1.2 Hz, 12.8 Hz, 1H), 1.86-1.71 (m, 1H), 1.69-1.49 (m, 4H), 1.43 (s, 9H), 0.95-0.80 (m, 6H) ppm. MS: M/e 243 (M+1)⁺.

### Intermediate 2: tert-butyl (2R,5S)-2,5-diethylpiperazine-1-carboxylate

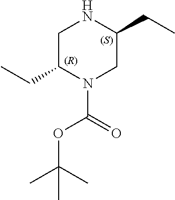

### Step A: methyl (S)-2-(benzylamino)butanoate

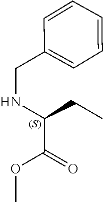

To a solution of compound 1 (250 g, 1.6 mol) in DCE (3500 mL) was added TEA (164 g, 1.6 mol), PhCHO (172 g, 1.6 mol) and NaBH(OAc)₃ (516 g, 2.4 mol) at ice-bath. The reaction mixture was stirred at rt for 5 h. The reaction mixture was quenched with Na₂CO₃ (1000 mL) and extracted with DCM (1500 mL×2). The combined organic layers were washed with brine (1000 mL) and concentrated. The residue was purified by chromatography on silica gel (PE:EA=20:1) to afford titled compound (315 g, 93.8%) as colorless oil. MS: M/e 208 (M+1)⁺

### Step B: methyl (S)-2-((R)—N-benzyl-2-((tert-butoxycarbonyl)amino)butanamido)butanoate

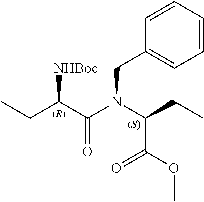

To a solution of methyl (S)-2-(benzylamino)butanoate (244 g, 1.2 mol) in DCM (7000 mL) was added (R)-2-((tert-butoxycarbonyl)amino)butanoic acid (285 g, 1.4 mol) and EDCI (113 g, 0.16 mol) at ice-bath. The reaction mixture was stirred for 30 min at rt. EDCI (113 g, 0.16 mol) was added. The reaction mixture was stirred for an additional hour and additional EDCI (113 g, 0.16 mol) was added. After the reaction mixture was stirred for 2 days at rt. The reaction mixture was poured into H₂O (2500 mL), and extracted with DCM (1500 mL×2). The combined organic layers were washed with brine (1000 mL) and concentrated. The residue was purified by chromatography on silica gel (PE:EA=10:1) to afford titled compound (152 g, 33%) as colorless oil. MS: M/e 393 (M+1)⁺

### Step C: (3R,6S)-1-benzyl-3,6-diethylpiperazine-2,5-dione

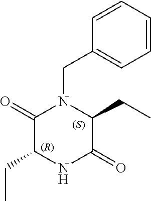

The reaction mixture of methyl (S)-2-((R)—N-benzyl-2-((tert-butoxycarbonyl)amino)butanamido)butanoate (143 g, 0.36 mol) in 4 N HCl/Dioxane (1500 mL) was stirred at rt for 5 h. Then the reaction mixture was basified to adjust pH 9.0 with sat. NaHCO₃ and stirred for 2 h. The mixture was extracted with DCM (1500 mL×2). The combined organic layers were washed with brine (2 L) and concentrated. The residue was purified by chromatography on silica gel (PE:EA=2:1) to afford titled compound (80 g, 85%) as an off-white solid. ¹H NMR (DMSO-d₆, 400 MHz): δ 8.23 (s, 1H), 7.36-7.23 (m, 5H), 5.10 (d, J=15.2 Hz, 1H), 4.15-4.13 (m, 1H), 4.08 (d, J=14.8 Hz 1H), 3.65-3.63 (m, 1H), 1.93-1.82 (m, 4H), 0.84-0.79 (m, 6H) ppm. MS: M/e 261 (M+1)⁺.

### Step D: (2S,5R)-1-benzyl-2,5-diethylpiperazine

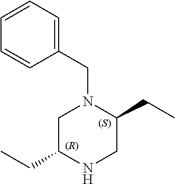

To a solution of (3R,6S)-1-benzyl-3,6-diethylpiperazine-2,5-dione (70 g, 0.27 mol) in THF (1000 mL) was added a solution of LiAlH₄ (51 g, 1.35 mol) at ice-bath. The reaction mixture was stirred at 70° C. for 4 h. The reaction mixture was cooled, added water (51.15 mL), 10% NaOH (51.15 mL) and water (153.45 mL). The mixture was stirred for 4 h. The mixture was filtered. The filtrate was concentrated to afford titled compound (62 g, 99.9%) which was used to the next step without further purification. MS: M/e 233 (M+1)⁺.

### Step E: tert-butyl (2R,5S)-4-benzyl-2,5-diethylpiperazine-1-carboxylate

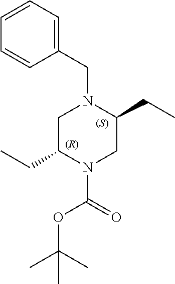

To a solution of (2S,5R)-1-benzyl-2,5-diethylpiperazine (62 g, 0.27 mol) in THF (1000 mL) was added Boc₂O (64 g, 0.29 mol) and DIEA (50 g, 0.39 mol) at ice-bath. The reaction mixture was stirred at rt overnight. The reaction mixture was poured into H₂O (300 mL), and extracted with DCM (1000 mL×2). The combined organic layers were washed with brine (1 L) and concentrated. The residue was purified by chromatography on silica gel (PE:EA=25:1) to afford titled compound (74 g, 88.7%) as colorless oil. ¹H NMR (DMSO-d₆, 400 MHz): δ7.34-7.23 (m, 5H), 3.88-3.84 (m, 2H), 3.67 (d, J=18.4 Hz, 1H), 3.50 (d, J=18.0 Hz, 1H), 3.06-3.04 (m, 1H), 2.58-2.50 (m, 1H), 2.27 (d, J=17.2 Hz, 1H), 1.82-1.75 (m, 1H), 1.59-1.45 (m, 13H), 0.88 (t, J=9.6 Hz, 3H), 0.74 (t, J=9.6 Hz, 3H) ppm. MS: M/e 333 (M+1)⁺.

### Step F: tert-butyl (2R,5S)-2,5-diethylpiperazine-1-carboxylate

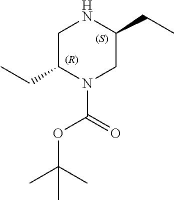

To a solution of tert-butyl (2R,5S)-4-benzyl-2,5-diethylpiperazine-1-carboxylate (70 g, 0.1 mol) in MeOH (500 mL) was added Pd/C (14 g) at rt. The reaction mixture was stirred at 30° C. overnight under H₂ (1 atm). The resulting mixture was filtered and concentrated to afford titled compound (50 g, 98%) as colorless oil. ¹H NMR (DMSO-d₆, 400 MHz): δ 3.76-3.74 (m, 1H), 3.63 (d, J=17.6 Hz, 1H), 3.00 (dd, J=5.2, 17.6 Hz, 1H), 2.89 (dd, J=6.0, 17.2 Hz, 1H), 2.57-2.56 (m, 1H), 2.41-2.24 (m, 1H), 1.79-1.72 (m, 1H), 1.57-1.46 (m, 3H), 1.38 (s, 9H), 0.88-0.77 (m, 6H) ppm. LCMS [column: Xbridge C18; column size: 4.6*50 mm, 3.5 um; mobile phase: from 95% water (0.1% TFA) and 5% CH₃CN to 5% water (0.1% TFA) and 95% CH₃CN in 6.5 min, finally under these conditions for 0.5 min.] purity is >95%, Rt=3.131 min; MS: M/e 243 (M+1)⁺.

### Intermediate 3: tert-butyl (2R,5S)-2-ethyl-5-methylpiperazine-1-carboxylate

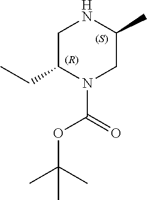

### Step A: methyl benzyl-L-alaninate

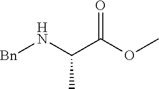

To a suspension of methyl L-alaninate hydrogen chloride (50 g, 0.36 mol) and K₂CO₃ (150 g, 1.08 mol) in MeCN (500 mL) was added BnBr (80 g, 0.47 mol) in drops at room temperature and the resulted mixture was stirred at room temperature for 16 hours. The mixture was filtered. The filter cake was washed with EtOAc (200 mL×3). The organics were combined and concentrated. The resulted residue was diluted with EtOAc (500 mL), washed with saturated aqueous solution of NaHCO₃ (200 mL), brine (200 mL×3), dried and concentrated. The resulted oil was purified by flash column chromatography to give the titled compound (28.0 g, 40%). MS: M/e 194 (M+1)⁺.

### Step B: methyl N-benzyl-N—((R)-2-((tert-butoxycarbonyl)amino)butanoyl)-L-alaninate

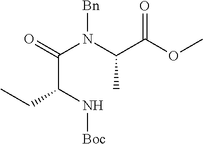 [SMILES: CC[C@@H](NC(=O)OC(C)(C)C)C(=O)N(Cc1ccccc1)[C@@H](C)C(=O)OC]

To a mixture of methyl benzyl-L-alaninate (27.0 g, 140 mmol), (R)-2-((tert-butoxycarbonyl)amino)butanoic acid (31.2 g, 154 mmol) and DIPEA (36.5 g, 280 mmol) in CH₂Cl₂ (300 mL) was added HATU (60.0 g, 158 mmol) in portions at 0° C. The resulted mixture was stirred at room temperature for 20 hours. The mixture was washed with saturated aqueous solution of NaHCO₃ (100 mL×2), brine (100 mL×2), dried over Na₂SO₄ and concentrated. The residue was purified by flash column chromatography to give the titled compound (45 g, 85%). MS: M/e 379 (M+1)⁺.

### Step C: methyl N—((R)-2-aminobutanoyl)-N-benzyl-L-alaninate

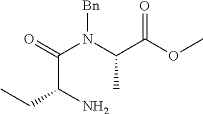 [SMILES: CC[C@@H](N)C(=O)N(Cc1ccccc1)[C@@H](C)C(=O)OC]

To a solution of methyl N-benzyl-N—((R)-2-((tert-butoxycarbonyl)amino)butanoyl)-L-alaninate (41.0 g, 108.5 mmol) in EtOAc (300 mL) was added HCl (100 mL, 4M in Dioxane) at room temperature and the mixture was stirred at room temperature for 3 days. The mixture was concentrated to dryness to give the titled compound (38.0 g, crude) which was used for the next step directly. MS: M/e 279 (M+1)⁺.

### Step D: (3R,6S)-1-benzyl-3-ethyl-6-methylpiperazine-2,5-dione

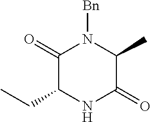 [SMILES: CC[C@H]1NC(=O)[C@H](C)N(Cc2ccccc2)C1=O]

To a mixture of methyl N—((R)-2-aminobutanoyl)-N-benzyl-L-alaninate hydrogen chloride (38.0 g, crude) in a mixed solvent EA/H₂O (200 mL/50 mL) was added NaHCO₃ (55 g, 650 mmol) in portions at room temperature. The resulted mixture was stirred at room temperature for 5 hours. Layers were separated. The aqueous layer was extracted with EtOAc (100 mL×3). The combined organic layers was washed with brine (200 mL×3), dried over Na₂SO₄ and concentrated. The resulting residue was purified by flash column chromatography to give the titled compound (24 g, 88% for 2 steps). MS: M/e 247 (M+1)⁺.

### Step E: (2S,5R)-1-benzyl-5-ethyl-2-methylpiperazine

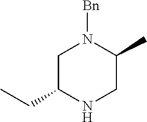 [SMILES: CC[C@@H]1CN(Cc2ccccc2)[C@@H](C)CN1]

A solution of borane in THF (1M, 480 mL) was slowly added to (3R,6S)-1-benzyl-3-ethyl-6-methylpiperazine-2,5-dione (23.6 g, 95.9 mmol) over 15 min. The resulted mixture was stirred at 70° C. for 72 hours. The mixture was cooled to 0° C., MeOH (100 mL) was added slowly and followed by HCl (5M, 40 mL) slowly. Large amount of bubble was found. After stirring for 30 min, the mixture was heated at 70° C. for 2 hours. The mixture was concentrated to dryness and purified by flash column chromatography to give the titled compound (21.5 g, crude) which was used for the next step directly. MS: M/e 219 (M+1)⁺.

### Step F: tert-butyl (2R,5S)-4-benzyl-2-ethyl-5-methylpiperazine-1-carboxylate

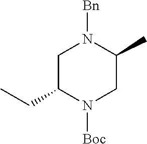 [SMILES: CC[C@@H]1CN(Cc2ccccc2)[C@@H](C)CN1C(=O)OC(C)(C)C]

To a solution of (2S,5R)-1-benzyl-5-ethyl-2-methylpiperazine (19.5 g, crude) in THF (150 mL) was added K₂CO₃ (24.5 g, 177 mmol) and H₂O (50 mL) at room temperature and followed by di-tert-butyl decarbonate (20.0 g, 91.7 mmol) in drops. The resulted mixture was stirred at room temperature for 16 hours. The mixture was diluted with of EtOAc (200 mL), washed with brine (100 mL×3), dried over Na₂SO₄, concentrated and purified by flash column chromatography to give the titled compound (23.8 g, 86% for 2 steps). MS: M/e 319 (M+1)⁺.

### Step G: tert-butyl (2R,5S)-2-ethyl-5-methylpiperazine-1-carboxylate

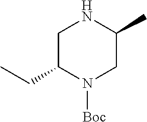 [SMILES: CC[C@@H]1CN[C@@H](C)CN1C(=O)OC(C)(C)C]

A mixture of tert-butyl (2R,5S)-4-benzyl-2-ethyl-5-methylpiperazine-1-carboxylate (23.8 g, 74.8 mmol), Pd/C (3.0 g), AcOH (2 mL) in MeOH (400 mL) was stirred at room temperature under H₂ for 16 hours. The mixture was filtered and the filtrate was concentrated. The resulted oil was treated with aqueous solution of NaOH (5M, 50 mL), extracted with CH₂Cl₂ (100 mL×3). The combined organic layers was washed with brine (100 mL×3), dried over Na₂SO₄, concentrated to dryness to give the titled compound (14.3 g, 84%). ¹H NMR (400 MHz, DMSO-d6) δ 3.42-3.23 (m, 1H), 3.04 (d, J=12.0 Hz, 1H), 2.64-2.46 (m, 3H), 2.15-2.05 (m, 1H), 1.99 (dd, J=12.8, 1.6 Hz, 1H), 1.41-1.24 (m, 1H), 1.16-1.01 (m, 1H), 0.97 (s, 9H), 0.60 (d, J=6.8 Hz, 3H), 0.36 (t, J=7.6 Hz, 3H). MS: M/e 229 (M+1)⁺.

### Intermediate 4: tert-butyl (2S,5R)-5-ethyl-2-methylpiperazine-1-carboxylate

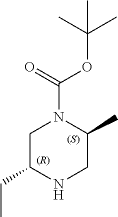

### Step A: methyl (R)-2-(benzylamino)butanoate

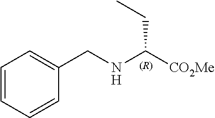

To a solution of methyl (R)-2-aminobutanoate hydrogen chloride (100 g, 651 mmol) and K₂CO₃ (225 g, 1.628 mol) in CH₃CN (700 mL) was added (bromomethyl)benzene (122.5 g, 716 mmol) dropwise at 0° C. The reaction mixture was stirred at room temperature for 12 hours. After filtration, the reaction solution was concentrated under reduced pressure. The resulting residue was purified by flash column chromatography to give the titled compound (70 g, 52%). MS: M/e 208 (M+1)⁺.

### Step B: methyl (R)-2-((S)—N-benzyl-2-((tert-butoxycarbonyl)amino)propanamido)butanoate

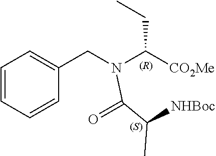

To a solution of methyl (R)-2-(benzylamino)butanoate (35 g, 169 mmol), (tert-butoxycarbonyl)-L-alanine (48 g, 254 mmol) and HATU (116 g, 304 mmol) in DCM (400 mL) was added NMM (43 g, 422 mmol). The reaction mixture solution was stirred at room temperature for 24 hours. The reaction was quenched with saturated NaCl (100 mL) at room temperature. The resulting mixture was extracted with DCM (300 mL×2). The combined organic layers were dried over Na₂SO₄ and concentrated under reduced pressure. The resulting residue was purified by flash column chromatography to give the titled compound (45 g, 70%). MS: M/e 379 (M+1)⁺.

### Step C: methyl (R)-2-((S)-2-amino-N-benzylpropanamido)butanoate hydrogen chloride

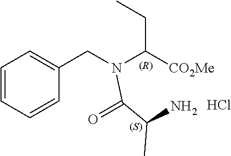

To a solution of methyl (R)-2-((S)—N-benzyl-2-((tert-butoxycarbonyl)amino)propanamido)butanoate (45 g, 119 mmol) in DCM (250 mL) at room temperature was added HCl (119 mL, 4 M in 1,4-dioxane). The reaction mixture was stirred at room temperature for 4 hours, concentrated under reduced pressure to give the titled compound (32 g, 86%). MS: M/e 279 (M+1)⁺.

### Step D: (3S,6R)-1-benzyl-6-ethyl-3-methylpiperazine-2,5-dione

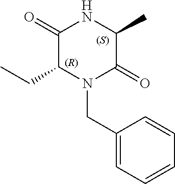

To a solution of methyl (R)-2-((S)-2-amino-N-benzylpropanamido)butanoate hydrogen chloride (32 g, 102 mmol) in NaHCO₃ (150 mL, 4M) was added EA (150 mL). The reaction mixture was stirred at room temperature for 2 hours. The combined organic layers were dried over Na₂SO₄ and concentrated under reduced pressure to give the titled compound (22 g, 87%). MS: M/e 247 (M+1)⁺.

### Step E: (2R,5S)-1-benzyl-2-ethyl-5-methylpiperazine

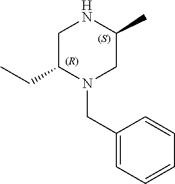

To a solution of (3S,6R)-1-benzyl-6-ethyl-3-methylpiperazine-2,5-dione (20 g, 81.3 mmol) in THE (300 mL) at 0° C. was added LiAlH₄ (6.2 g, 163 mmol) in portion. The reaction mixture was stirred at 70° C. for 36 hours and added H₂O (6.2 mL), followed NaOH (6.2 mL, 20%) and H₂O (12.4 mL) at 0° C. to quench the reaction solution. After filtration, the combined organic layers were dried over Na₂SO₄ and concentrated under reduced pressure to give the titled compound (11 g, 61%). MS: M/e 219 (M+1)⁺.

### Step F: tert-butyl (2S,5R)-4-benzyl-5-ethyl-2-methylpiperazine-1-carboxylate

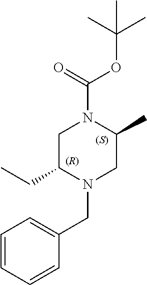

To a solution of (2R,5S)-1-benzyl-2-ethyl-5-methylpiperazine (11 g, 50 mmol) and Boc₂O (12 g, 55 mmol) in DCM (200 mL) was added Et₃N (7.6 g, 75 mmol). The reaction mixture was stirred at room temperature for 12 hours. The reaction was quenched with saturated NaCl (100 mL) at room temperature. The resulting mixture was extracted with DCM (200 mL×2). The combined organic layers were dried over Na₂SO₄ and concentrated under reduced pressure. The resulting residue was purified by flash column chromatography to give the titled compound (14 g, 69%). MS: M/e 319 (M+1)⁺.

### Step G: tert-butyl (2S,5R)-5-ethyl-2-methylpiperazine-1-carboxylate

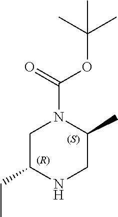

To a solution of tert-butyl (2S,5R)-4-benzyl-5-ethyl-2-methylpiperazine-1-carboxylate (10 g, 31.4 mmol) and Pd/C (2.5 g, 10% in water) in MeOH (60 mL) at room temperature was added AcOH (2 mL). The resulting mixture was degassed 3 times under H₂ atmosphere, and stirred at room temperature under H₂ atmosphere for 12 hours. After filtration, the combined organic layers were concentrated under reduced pressure to give the crude product (AcOH salt). The crude product was basified by Na₂CO₃ (4M) to pH ˜10 and extracted with EA (80 mL×3). The combined organic layers were washed with brine (20 mL×3), dried over Na₂SO₄ and concentrated under reduced pressure to give the titled compound (5.5 g, 77%). ¹H NMR (400 MHz, CDCl₃) δ 4.21-4.09 (m, 1H), 3.72-3.69 (d, J=12.9 Hz, 1H), 3.24-3.19 (dd, J=13.7, 4.0 Hz, 1H), 3.14-3.10 (dd, J=12.8, 4.7 Hz, 1H), 2.86-2.75 (m, 1H), 2.53-2.49 (dd, J=12.8, 2.7 Hz, 1H), 2.33-2.12 (m, 1H), 1.65-1.54 (m, 2H), 1.46 (s, 9H), 1.25-1.24 (d, J=4.0 Hz, 3H), 0.98-0.91 (m, 3H) ppm. MS: M/e 229 (M+1)⁺.

### Intermediate 5: 2-(6-((2S,5R)-2,5-dimethylpiperazin-1-yl)-3,9-dimethyl-2-oxo-3,9-dihydro-2H-purin-8-yl)acetonitrile

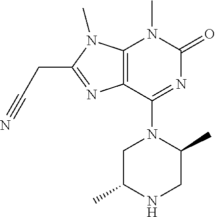 [SMILES: C[C@@H]1CN(c2nc(=O)n(C)c3c2nc(CC#N)n3C)[C@@H](C)CN1]

### Step A: tert-butyl (2R,5S)-4-(2-chloro-9H-purin-6-yl)-2,5-dimethylpiperazine-1-carboxylate

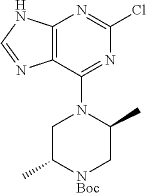 [SMILES: C[C@@H]1CN(c2nc(Cl)nc3[nH]cnc23)[C@@H](C)CN1C(=O)OC(C)(C)C]

To a solution of 2,6-dichloro-9H-purine (200 g, 529 mmol) in EtOH (2400 mL) was added tert-butyl (2R,5S)-2,5-dimethylpiperazine-1-carboxylate (238 g, 555 mmol), DIPEA (204.6 g, 794 mmol). The reaction was stirred at 80° C. for 16 h. The mixture was concentrated in vacuo. The residue was added DCM and washed with H₂O. The organic phase was dried over Na₂SO₄ and concentrated in vacuo to give the titled compound (387 g, 99%). ¹H NMR (400 MHz, CD₃OD) δ 7.97 (s, 1H), 6.17-5.05 (m, 2H), 4.43 (d, J=20.5 Hz, 1H), 3.83 (d, J=13.6 Hz, 1H), 3.80-3.35 (m, 2H), 1.50 (d, J=3.6 Hz, 9H), 1.30 (d, J=6.8 Hz, 3H), 1.18 (d, J=6.8 Hz, 3H) ppm. MS: M/e 367 (M+1)⁺.

### Step B: tert-butyl(2R,5S)-4-(2-chloro-9-methyl-9H-purin-6-yl)-2,5-dimethylpiperazine-1-carboxylate

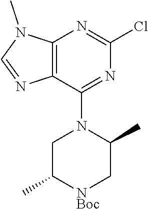 [SMILES: C[C@@H]1CN(c2nc(Cl)nc3c2ncn3C)[C@@H](C)CN1C(=O)OC(C)(C)C]

To a solution of tert-butyl (2R,5S)-4-(2-chloro-9H-purin-6-yl)-2,5-dimethylpiperazine-1-carboxylate (387 g, 1.06 mol) in acetone (2500 mL) was added K₂CO₃ (295 g, 2.12 mol) and CH₃I (181 g, 1.27 mol). The reaction was stirred at room temperature overnight. The mixture was filtered. The filter cake was washed with DCM. The filtrate was concentrated in vacuo. The residue was dissolved in DCM and washed by H₂O. The organic phase was dried over Na₂SO₄ and concentrated in vacuo. The residue was added a solution of PE/EtOAc=2/1 and stirred for 15 mins. The mixture was filtered. The filter cake was dried in vacuo to give the titled compound (350 g, 87%). ¹H NMR (400 MHz, CD3OD) δ 7.95 (s, 1H), 6.19-4.92 (m, 2H), 4.43 (d, J=19.6 Hz, 1H), 3.82 (d, J=13.8 Hz, 1H), 3.75 (s, 3H), 3.70-3.34 (m, 2H), 1.49 (s, 9H), 1.30 (d, J=6.7 Hz, 3H), 1.17 (d, J=6.8 Hz, 3H) ppm. MS: M/e 381 (M+1)⁺.

### Step C: tert-butyl(2R,5S)-2,5-dimethyl-4-(9-methyl-2-oxo-3,9-dihydro-2H-purin-6-yl) piperazine-1-carboxylate

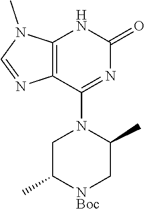 [SMILES: C[C@@H]1CN(c2nc(=O)[nH]c3c2ncn3C)[C@@H](C)CN1C(=O)OC(C)(C)C]

To a solution of tert-butyl (2R,5S)-4-(2-chloro-9-methyl-9H-purin-6-yl)-2,5-dimethylpiperazine-1-carboxylate (100 g, 263 mmol) in dioxane/H₂O (1400 mL/170 mL) was added 2-Di-tert-butylphosphino-2′,4′,6′-triisopropylbiphenyl (11.2 g, 26.3 mmol) KOH (44 g, 789 mmol) and Pd₂(dba)₃ (2.41 g, 2.63 mmol). The reaction was stirred at 90° C. overnight. The mixture was cooled down to rt, added EtOAc (1.5 L) and separated. The H₂O phase was filtered. The filtrate was adjusted pH 6-7 and extracted by DCM. The organic phase was dried over Na₂SO₄ and concentrated in vacuo to give the titled compound (100.8 g). ¹H NMR (400 MHz, CD3OD) δ 7.56 (s, 1H), 5.67-4.90 (m, 2H), 4.36 (d, J=22.3 Hz, 1H), 3.76 (d, J=13.5 Hz, 1H), 3.62 (s, 3H), 3.57-3.33 (m, 2H), 1.49 (d, J=4.2 Hz, 9H), 1.24 (d, J=7.0 Hz, 3H), 1.15 (d, J=6.8 Hz, 3H) ppm. MS: M/e 363 (M+1)⁺.

### Step D: tert-butyl (2R,5S)-4-(3,9-dimethyl-2-oxo-3,9-dihydro-2H-purin-6-yl)-2,5-dimethylpiperazine-1-carboxylate

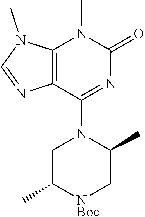 [SMILES: C[C@@H]1CN(c2nc(=O)n(C)c3c2ncn3C)[C@@H](C)CN1C(=O)OC(C)(C)C]

To a mixture of tert-butyl (2R,5S)-2,5-dimethyl-4-(9-methyl-2-oxo-3,9-dihydro-2H-purin-6-yl)piperazine-1-carboxylate (96 g, 265 mmol) in dioxane (2000 mL) and was added Cs₂CO₃ (432 g, 1.33 mol) and CH₃I (376 g, 2.65 mol). The reaction was stirred at room temperature overnight and 70° C. for 2 h. The reaction mixture was filtered. The filtrate was removed under reduced pressure to dryness. The crude product dissolved in DCM and washed with H₂O. The organic phase was dried over Na₂SO₄ and concentrated in vacuo. The residue was added PE/EtOAc=500 ml/500 ml and stirred for 0.5 h. The mixture was filtered. The filter cake was dried to give the titled compound (52 g, 53%). ¹H NMR (400 MHz, CDCl₃) δ 7.26 (s, 1H), 5.71 (s, 0.5H), 5.52 (s, 0.5H), 4.51 (s, 0.5H), 4.30 (s, 0.5H), 3.96 (d, J=6.9 Hz, 3H), 3.91-3.55 (m, 5H), 3.31 (m, 2H), 1.47 (s, 9H), 1.35-1.23 (m, 3H), 1.15 (d, J=6.5 Hz, 3H) ppm. MS: M/e 376 (M+1)⁺.

### Step E: tert-butyl(2R,5S)-4-(8-formyl-3,9-dimethyl-2-oxo-3,9-dihydro-2H-purin-6-yl)-2,5-dimethylpiperazine-1-carboxylate

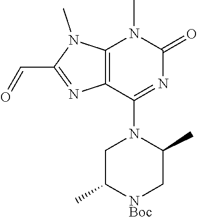 [SMILES: C[C@@H]1CN(c2nc(=O)n(C)c3c2nc(C=O)n3C)[C@@H](C)CN1C(=O)OC(C)(C)C]

To a solution of tert-butyl (2R,5S)-4-(3,9-dimethyl-2-oxo-3,9-dihydro-2H-purin-6-yl)-2,5-dimethylpiperazine-1-carboxylate (20 g, 53.2 mmol) in THE (1.2 L) was added LDA (106 mL, 2M, 212 mmol) at below −60° C. The reaction was stirred at −65° C. for 1 hour, then added DMF (19.4 g, 266 mmol) at −78° C. The reaction was stirred at −60° C. for another 1 hour. The reaction solvent was quenched by saturated NH₄Cl and extracted by EtOAc. The organic phase was dried over Na₂SO₄ and concentrated in vacuo to give the titled compound (23.1 g). ¹H NMR (400 MHz, CD₃OD) δ 9.68 (s, 1H), 6.35-5.72 (m, 1H), 5.40-4.65 (m, 1H), 4.39 (s, 1H), 4.07 (s, 3H), 3.80 (s, 3H), 3.78-3.35 (m, 3H), 1.49 (s, 9H), 1.33-1.24 (m, 3H), 1.18-1.07 (m, 3H) ppm. MS: M/e 405 (M+1)⁺.

### Step F: tert-butyl (2R,5S)-4-(8-(hydroxymethyl)-3,9-dimethyl-2-oxo-3,9-dihydro-2H-purin-6-yl)-2,5-dimethylpiperazine-1-carboxylate

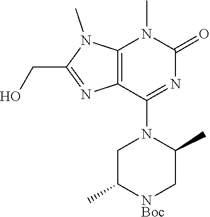 [SMILES: C[C@@H]1CN(c2nc(=O)n(C)c3c2nc(CO)n3C)[C@@H](C)CN1C(=O)OC(C)(C)C]

NaBH₄ (1.5 g, 40 mol) was added to tert-butyl (2R,5S)-4-(8-formyl-3,9-dimethyl-2-oxo-3,9-dihydro-2H-purin-6-yl)-2,5-dimethylpiperazine-1-carboxylate (23.1 g, 57 mmol) in MeOH (10 mL) at 0° C. for 5 min. The reaction solvent was quenched with water and extracted with DCM. The organic layer was purified by flash column chromatography (DCM/MeOH) to give the titled compound (15.6 g, 68%). MS: M/e 406 (M+1)⁺.

### Step G: tert-butyl (2R,5S)-4-(8-(chloromethyl)-3,9-dimethyl-2-oxo-3,9-dihydro-2H-purin-6-yl)-2,5-dimethylpiperazine-1-carboxylate

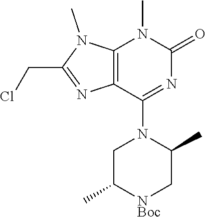 [SMILES: C[C@@H]1CN(c2nc(=O)n(C)c3c2nc(CCl)n3C)[C@@H](C)CN1C(=O)OC(C)(C)C]

To a solution of tert-butyl (2R,5S)-4-(8-(hydroxymethyl)-3,9-dimethyl-2-oxo-3,9-dihydro-2H-purin-6-yl)-2,5-dimethylpiperazine-1-carboxylate (12 g, 29.6 mmol) in DCM (150 mL) was added SOCl₂ (4.2 g, 35.5 mmol) at 0° C. The reaction was stirred at rt for 5 min. The reaction solvent was quenched with water and washed with water, sat. NaHCO₃ and sat. NaCl. The organic layer was dried over Na₂SO₄ and concentrated in vacuo. The crude product was used in next step directly.

### Step H: tert-butyl (2R,5S)-4-(8-(cyanomethyl)-3,9-dimethyl-2-oxo-3,9-dihydro-2H-purin-6-yl)-2,5-dimethylpiperazine-1-carboxylate

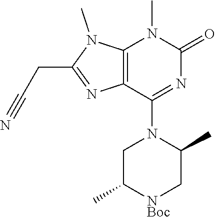 [SMILES: C[C@@H]1CN(c2nc(=O)n(C)c3c2nc(CC#N)n3C)[C@@H](C)CN1C(=O)OC(C)(C)C]

To the solution of tert-butyl (2R,5S)-4-(8-(chloromethyl)-3,9-dimethyl-2-oxo-3,9-dihydro-2H-purin-6-yl)-2,5-dimethylpiperazine-1-carboxylate from last step in MeCN (400 mL) were added (14.6 g, 148 mmol) and CS₂CO₃ (28.9 g, 88.8 mmol). The resulting mixture was stirred at 60° C. for 1 hours. The mixture was added H₂O and extracted by EtOAc. The organic phase was dried over Na₂SO₄ and concentrated in vacuo. The crude product was purified by flash column chromatography (DCM/MeOH) to give the titled compound (5.8 g, two steps 47%). ¹H NMR (400 MHz, CD₃OD) δ 6.25-5.64 (m, 1H), 5.36-4.66 (m, 1H), 4.38 (s, 1H), 4.23 (s, 2H), 3.94 (s, 3H), 3.80 (s, 3H), 3.75-3.33 (m, 3H), 1.48 (s, 9H), 1.28 (dd, J=19.6, 6.3 Hz, 3H), 1.15 (d, J=6.7 Hz, 3H) ppm. MS: M/e 416 (M+1)⁺.

### Step I: 2-(6-((2S,5R)-2,5-dimethylpiperazin-1-yl)-3,9-dimethyl-2-oxo-3,9-dihydro-2H-purin-8-yl)acetonitrile

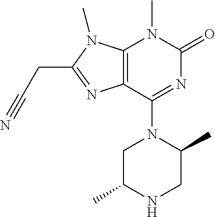 [SMILES: C[C@@H]1CN(c2nc(=O)n(C)c3c2nc(CC#N)n3C)[C@@H](C)CN1]

To a stirred solution of tert-butyl (2R,5S)-4-(8-(cyanomethyl)-3,9-dimethyl-2-oxo-3,9-dihydro-2H-purin-6-yl)-2,5-dimethylpiperazine-1-carboxylate (1.3 g, 3 mmol) in CH₂Cl₂ (15 mL) was added TFA (4 mL). After stirred for 3 hours, the reaction mixture was concentrated to give the residue, which was basified to pH=10˜12 with aq.K₂CO₃, extracted with CH₂Cl₂/IPA (3/1, 50 mL×6). The combined organic layers were dried over Na₂SO₄, concentrated to give the titled compound (700 mg, 71%). MS: M/e 316 (M+1)⁺.

### Intermediate 6: 2-(6-((2S,5R)-2,5-dimethylpiperazin-1-yl)-9-ethyl-3-methyl-2-oxo-3,9-dihydro-2H-purin-8-yl)acetonitrile

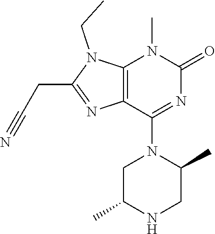 [SMILES: CCn1c(CC#N)nc2c(N3C[C@@H](C)NC[C@@H]3C)nc(=O)n(C)c21]

### Step A: tert-butyl (2R,5S)-4-(2-chloro-9-ethyl-9H-purin-6-yl)-2,5-dimethylpiperazine-1-carboxylate

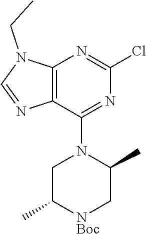 [SMILES: CCn1cnc2c(N3C[C@@H](C)N(C(=O)OC(C)(C)C)C[C@@H]3C)nc(Cl)nc21]

To a solution of tert-butyl (2R,5S)-4-(2-chloro-9H-purin-6-yl)-2,5-dimethylpiperazine-1-carboxylate (6.97 g, 19 mmol) in acetone (100 mL) was added K₂CO₃ (5.2 g, 38 mmol), followed by EtI (5.9 g, 38 mmol). After the addition, the reaction mixture was stirred overnight. The reaction mixture was filtered, and the filtrate was concentrated to give the residue, which was treated with CH₂Cl₂ (100 mL), washed with brine, dried over Na₂SO₄, concentrated to give the titled compound (6.5 g, 87%). MS: M/e 395 (M+1)⁺.

### Step B: tert-butyl (2R,5S)-4-(9-ethyl-2-oxo-3,9-dihydro-2H-purin-6-yl)-2,5-dimethylpiperazine-1-carboxylate

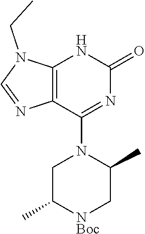 [SMILES: CCn1cnc2c(N3C[C@@H](C)N(C(=O)OC(C)(C)C)C[C@@H]3C)nc(=O)[nH]c21]

To a solution of tert-butyl (2R,5S)-4-(2-chloro-9-ethyl-9H-purin-6-yl)-2,5-dimethylpiperazine-1-carboxylate (6.5 g, 16.4 mmol) in dioxane (80 mL) was added aq.KOH (3.68 g, 65.8 mmol in 15 mL H₂O), Pd₂(dba)₃ (751 mg, 0.82 mmol) and t-BuXphOS (695 mg, 16.4 mmol). After the addition, the reaction mixture was stirred for an hour at 100° C. The reaction mixture was diluted with H₂O (80 mL), extracted with EtOAc (60 mL×3). The organic layers were discarded and the aqueous layer was acidified to pH=6˜7 with aq.citric acid and extracted with EtOAc (100 mL×3). The combined organic layers were washed with brine, dried over Na₂SO₄, concentrated to give the titled compound (5.3 g, 86%). MS: M/e 377 (M+1)⁺.

### Step C: tert-butyl (2R,5S)-4-(9-ethyl-3-methyl-2-oxo-3,9-dihydro-2H-purin-6-yl)-2,5-dimethylpiperazine-1-carboxylate

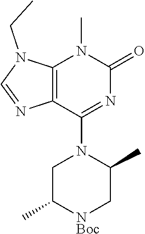 [SMILES: CCn1cnc2c(N3C[C@@H](C)N(C(=O)OC(C)(C)C)C[C@@H]3C)nc(=O)n(C)c21]

To a stirred solution of tert-butyl (2R,5S)-4-(9-ethyl-2-oxo-3,9-dihydro-2H-purin-6-yl)-2,5-dimethylpiperazine-1-carboxylate (5.3 g, 14.1 mmol) in dioxane (500 mL) was added Cs₂CO₃ (13.7 g, 42 mmol), followed by Mel (20 g, 0.14 mmol). After the addition, the reaction mixture was stirred for 3 days at RT and stirred at 80° C. for 3 hours. The reaction mixture was filtered, and the filtrate was concentrated to give the residue, which was purified by flash column chromatography to give the titled compound (3.32 g, 60%). MS: M/e 391 (M+1)⁺.

### Step D: tert-butyl (2R,5S)-4-(9-ethyl-8-formyl-3-methyl-2-oxo-3,9-dihydro-2H-purin-6-yl)-2,5-dimethylpiperazine-1-carboxylate

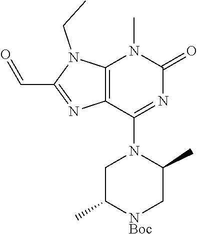 [SMILES: CCn1c(C=O)nc2c(N3C[C@@H](C)N(C(=O)OC(C)(C)C)C[C@@H]3C)nc(=O)n(C)c21]

To a stirred solution of tert-butyl (2R,5S)-4-(9-ethyl-3-methyl-2-oxo-3,9-dihydro-2H-purin-6-yl)-2,5-dimethylpiperazine-1-carboxylate (3.32 g, 8.51 mmol) in THE (300 mL) was added dropwise LDA (2.0 M, 17 mL, 34 mmol) at −78° C. After stirred for an hour at that temperature, DMF (3.06 g, 42.6 mmol) was added dropwise, then the mixture was allowed warm to RT. The reaction was quenched with aq.NH₄Cl, extracted with EtOAc (200 mL×2). The combined organic layers were washed with brine, dried over Na₂SO₄, concentrated to give the titled compound (crude, 100%), which was directly used to the next step.

### Step E: tert-butyl (2R,5S)-4-(9-ethyl-8-(hydroxymethyl)-3-methyl-2-oxo-3,9-dihydro-2H-purin-6-yl)-2,5-dimethylpiperazine-1-carboxylate

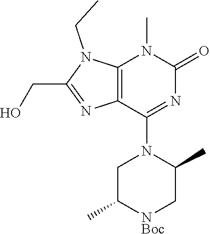 [SMILES: CCn1c(CO)nc2c(N3C[C@@H](C)N(C(=O)OC(C)(C)C)C[C@@H]3C)nc(=O)n(C)c21]

To a stirred solution of tert-butyl (2R,5S)-4-(9-ethyl-8-formyl-3-methyl-2-oxo-3,9-dihydro-2H-purin-6-yl)-2,5-dimethylpiperazine-1-carboxylate (crude, 8.51 mmol) in MeOH (20 mL) was added NaBH₄ (224 mg, 6 mmol). After stirred for 10 min, the reaction was quenched with H₂O, extracted with CH₂Cl₂ (50 mL×3). The combined organic layers were washed with brine, dried over Na₂SO₄, concentrated and purified by flash column chromatography to give the titled compound (2.9 g, 81%).

### Step F: tert-butyl (2R,5S)-4-(9-ethyl-3-methyl-8-(((methylsulfonyl)oxy)methyl)-2-oxo-3,9-dihydro-2H-purin-6-yl)-2,5-dimethylpiperazine-1-carboxylate

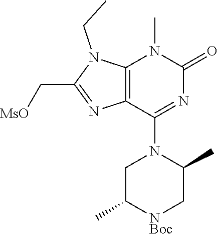 [SMILES: CCn1c(COS(C)(=O)=O)nc2c(N3C[C@@H](C)N(C(=O)OC(C)(C)C)C[C@@H]3C)nc(=O)n(C)c21]

To a stirred solution of tert-butyl (2R,5S)-4-(9-ethyl-8-(hydroxymethyl)-3-methyl-2-oxo-3,9-dihydro-2H-purin-6-yl)-2,5-dimethylpiperazine-1-carboxylate (2.9 g, 6.9 mmol) in CH₂Cl₂ (30 mL) was added Et₃N (1.4 g, 13.8 mmol), followed by MsCl (1.2 g, 10.4 mmol). After the addition, the reaction mixture was stirred for 2 hours, the reaction mixture was washed with H₂O (20 mL), extracted with CH₂Cl₂ (30 mL×2). The combined organic layers were washed with brine, dried over Na₂SO₄, concentrated to give the titled compound (2.4 g, 70%).

### Step G: tert-butyl (2R,5S)-4-(8-(cyanomethyl)-9-ethyl-3-methyl-2-oxo-3,9-dihydro-2H-purin-6-yl)-2,5-dimethylpiperazine-1-carboxylate

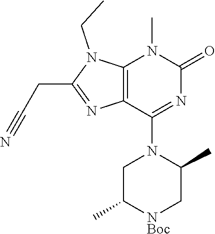 [SMILES: CCn1c(CC#N)nc2c(N3C[C@@H](C)N(C(=O)OC(C)(C)C)C[C@@H]3C)nc(=O)n(C)c21]

To a stirred solution of tert-butyl (2R,5S)-4-(9-ethyl-3-methyl-8-(((methylsulfonyl)oxy)methyl)-2-oxo-3,9-dihydro-2H-purin-6-yl)-2,5-dimethylpiperazine-1-carboxylate (2.4 g, 4.8 mmol) in CH₃CN (30 mL) was added Cs₂CO₃ (3.2 g, 9.6 mmol), followed by TMSCN (0.95 g, 9.6 mmol). After the addition, the reaction mixture was stirred at 70° C. for 2 hours. The reaction mixture was poured into H₂O (50 mL), extracted with EtOAc (30 mL×3). The combined organic layers were washed with brine, dried over Na₂SO₄, concentrated and purified by flash column chromatography to give the titled compound (1.3 g, 63%).

### Step H: 2-(6-((2S,5R)-2,5-dimethylpiperazin-1-yl)-9-ethyl-3-methyl-2-oxo-3,9-dihydro-2H-purin-8-yl)acetonitrile

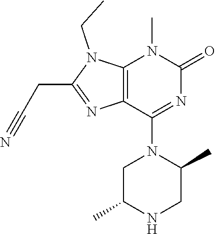 [SMILES: CCn1c(CC#N)nc2c(N3C[C@@H](C)NC[C@@H]3C)nc(=O)n(C)c21]

To a stirred solution of tert-butyl (2R,5S)-4-(8-(cyanomethyl)-9-ethyl-3-methyl-2-oxo-3,9-dihydro-2H-purin-6-yl)-2,5-dimethylpiperazine-1-carboxylate (1.3 g, 3 mmol) in CH₂Cl₂ (15 mL) was added TFA (4 mL). After stirred for 3 hours, the reaction mixture was concentrated to give the residue, which was basified to pH=10˜12 with aq.K₂CO₃, extracted with CH₂Cl₂/IPA (3/1, 50 mL×6). The combined organic layers were dried over Na₂SO₄, concentrated to give the titled compound (700 mg, 71%). MS: M/e 330 (M+1)⁺.

### Compound A1: 2-(7-((2S,5R)-2,5-dimethyl-4-(1-(quinoxalin-6-yl)ethyl)piperazin-1-yl)-3,4-dimethyl-5-oxo-4,5-dihydro-3H-imidazo[4,5-b]pyridin-2-yl)acetonitrile

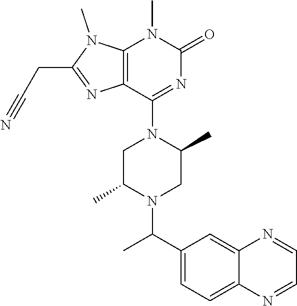 [SMILES: CC(c1ccc2nccnc2c1)N1C[C@H](C)N(c2nc(=O)n(C)c3c2nc(CC#N)n3C)C[C@H]1C]

### Step A: tert-butyl (2R,5S)-4-(5-chloro-3H-imidazo[4,5-b]pyridin-7-yl)-2,5-dimethylpiperazine-1-carboxylate

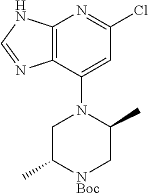 [SMILES: C[C@@H]1CN(c2cc(Cl)nc3[nH]cnc23)[C@@H](C)CN1C(=O)OC(C)(C)C]

To a solution of 5,7-dichloro-3H-imidazo[4,5-b]pyridine (2 g, 10.6 mmol) in DMSO (20 mL) were added tert-butyl (2R,5S)-2,5-dimethylpiperazine-1-carboxylate (11.4 g, 53.2 mmol), DIPEA (6.8 g, 53.2 mmol). The resulting mixture was stirred at 160° C. for 30 hours in sealed tube. The mixture was diluted with EtOAc then washed with water. The reaction solvent was concentrated to dryness. The resulting residue was purified by flash column chromatography to give the titled compound (3.2 g, 82%). MS: M/e 366 (M+1)⁺.

### Step B: tert-butyl (2R,5S)-4-(5-chloro-3-methyl-3H-imidazo[4,5-b]pyridin-7-yl)-2,5-dimethylpiperazine-1-carboxylate

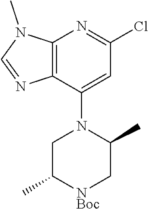 [SMILES: C[C@@H]1CN(c2cc(Cl)nc3c2ncn3C)[C@@H](C)CN1C(=O)OC(C)(C)C]

To a solution of tert-butyl (2R,5S)-4-(5-chloro-3H-imidazo[4,5-b]pyridin-7-yl)-2,5-dimethylpiperazine-1-carboxylate (3.2 g, 8.22 mmol) in acetone (50 mL) were added K₂CO₃ (2.3 g, 16.4 mmol) and CH₃I (2.3 g, 16.4 mmol). The resulting mixture was stirred at rt overnight. The organic layer was concentrated to dryness. The resulting residue was purified by flash column chromatography to give the titled compound (2.5 g, 76%). MS: M/e 380 (M+1)⁺.

### Step C: methyl 7-((2S,5R)-4-(tert-butoxycarbonyl)-2,5-dimethylpiperazin-1-yl)-5-chloro-3-methyl-3H-imidazo[4,5-b]pyridine-2-carboxylate

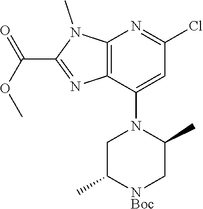 [SMILES: COC(=O)c1nc2c(N3C[C@@H](C)N(C(=O)OC(C)(C)C)C[C@@H]3C)cc(Cl)nc2n1C]

To a solution of tert-butyl (2R,5S)-4-(5-chloro-3-methyl-3H-imidazo[4,5-b]pyridin-7-yl)-2,5-dimethylpiperazine-1-carboxylate (1 g, 2.63 mmol) in THE (15 mL) were added n-BuLi (2.5 mL, 1.6M, 3.95 mmol) at −78° C. The resulting mixture was stirred at −78° C. for 1 h and warmed to −40° C. for another 1 h, then added methyl carbonochloridate (1.98 g, 21.1 mmol) at −78° C. The reaction solvent was stirred at −78° C. for another 10 min. The reaction solvent was quenched by saturated NH₄Cl and extracted by EtOAc. The crude product was concentrated to dryness. The resulting residue was purified by flash column chromatography to give the titled compound (700 mg, 61%). MS: M/e 438 (M+1)⁺.

### Step D: 7-((2S,5R)-4-(tert-butoxycarbonyl)-2,5-dimethylpiperazin-1-yl)-3-methyl-5-oxo-4,5-dihydro-3H-imidazo[4,5-b]pyridine-2-carboxylic acid

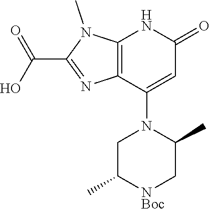 [SMILES: C[C@@H]1CN(c2cc(=O)[nH]c3c2nc(C(=O)O)n3C)[C@@H](C)CN1C(=O)OC(C)(C)C]

To a solution of methyl 7-((2S,5R)-4-(tert-butoxycarbonyl)-2,5-dimethylpiperazin-1-yl)-5-chloro-3-methyl-3H-imidazo[4,5-b]pyridine-2-carboxylate (0.9 g, 2.01 mmol) in dioxane (15 mL) were added t-BuXphos (88 mg, 0.2 mmol) KOH (346 mg, 6.2 mmol in 1.8 mL water) and Pd₂(dba)₃ (94 mg, 0.1 mmol). The resulting mixture was stirred at 90° C. for 4 hours. The reaction solvent was poured into water and extracted by EtOAc (discard). The aqueous layer was adjusted to pH=3 with citric acid. The aqueous layer was extracted with EtOAc to give the titled compound (900 mg, crude). MS: M/e 406 (M+1)⁺.

### Step E: methyl 7-((2S,5R)-4-(tert-butoxycarbonyl)-2,5-dimethylpiperazin-1-yl)-3,4-dimethyl-5-oxo-4,5-dihydro-3H-imidazo[4,5-b]pyridine-2-carboxylate

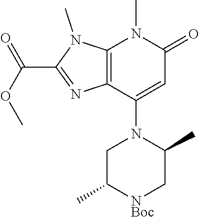 [SMILES: COC(=O)c1nc2c(N3C[C@@H](C)N(C(=O)OC(C)(C)C)C[C@@H]3C)cc(=O)n(C)c2n1C]

To a solution of 7-((2S,5R)-4-(tert-butoxycarbonyl)-2,5-dimethylpiperazin-1-yl)-3-methyl-5-oxo-4,5-dihydro-3H-imidazo[4,5-b]pyridine-2-carboxylic acid (700 mg, 1.73 mmol) in dioxane (20 mL) was added Cs₂CO₃ (1.69 g, 5.19 mmol) and CH₃I (736 mg, 5.19 mmol). The resulting mixture was stirred at 90° C. overnight. The reaction solvent was removed under reduced pressure to dryness. The crude product was purified by flash column chromatography to give the titled compound (163 mg, 22%). MS: M/e 434 (M+1)⁺.

### Step F: tert-butyl (2R,5S)-4-(2-(hydroxymethyl)-3,4-dimethyl-5-oxo-4,5-dihydro-3H-imidazo[4,5-b]pyridin-7-yl)-2,5-dimethylpiperazine-1-carboxylate

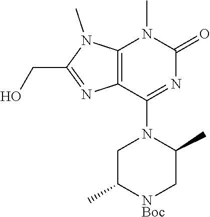 [SMILES: C[C@@H]1CN(c2nc(=O)n(C)c3c2nc(CO)n3C)[C@@H](C)CN1C(=O)OC(C)(C)C]

To a solution of methyl 7-((2S,5R)-4-(tert-butoxycarbonyl)-2,5-dimethylpiperazin-1-yl)-3,4-dimethyl-5-oxo-4,5-dihydro-3H-imidazo[4,5-b]pyridine-2-carboxylate (190 mg, 0.44 mmol) in THF (10 mL) were added NaBH₄ (34 mg, 0.88 mmol). The resulting mixture was stirred at 50° C. overnight. Then added NaBH₄ (34 mg, 0.88 mmol), the resulting mixture was stirred at 60° C. overnight. The reaction solvent was quenched by water and extracted with (DCM/IPA=4/1). The residue was purified by Prep-TLC (DCM:MeOH=20:1) to give the titled compound (130 mg, 73%) as a white solid. MS: M/e 406 (M+1)⁺.

### Step G: tert-butyl (2R,5S)-4-(2-(chloromethyl)-3,4-dimethyl-5-oxo-4,5-dihydro-3H-imidazo[4,5-b]pyridin-7-yl)-2,5-dimethylpiperazine-1-carboxylate

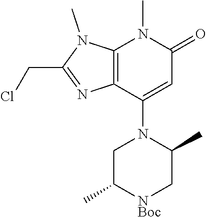 [SMILES: C[C@@H]1CN(c2cc(=O)n(C)c3c2nc(CCl)n3C)[C@@H](C)CN1C(=O)OC(C)(C)C]

To a solution of tert-butyl (2R,5S)-4-(2-(hydroxymethyl)-3,4-dimethyl-5-oxo-4,5-dihydro-3H-imidazo[4,5-b]pyridin-7-yl)-2,5-dimethylpiperazine-1-carboxylate (80 mg, 0.197 mmol) in DCM (20 mL) were added SOCl₂ (90 mg). The resulting mixture was stirred at RT for 5 min. The reaction solvent was washed with water, saturated NaHCO₃ and saturated NaCl. The organic layer was concentrated to give the titled compound which was used to next step directly without further purification.

### Step H: tert-butyl (2R,5S)-4-(2-(cyanomethyl)-3,4-dimethyl-5-oxo-4,5-dihydro-3H-imidazo[4,5-b]pyridin-7-yl)-2,5-dimethylpiperazine-1-carboxylate

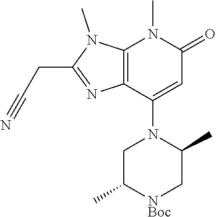 [SMILES: C[C@@H]1CN(c2cc(=O)n(C)c3c2nc(CC#N)n3C)[C@@H](C)CN1C(=O)OC(C)(C)C]

To a solution of tert-butyl (2R,5S)-4-(2-(chloromethyl)-3,4-dimethyl-5-oxo-4,5-dihydro-3H-imidazo[4,5-b]pyridin-7-yl)-2,5-dimethylpiperazine-1-carboxylate (crude) in CH₃CN (3 mL) were added TMSCN (80 mg, 0.79 mmol) Cs₂CO₃ (192 mg, 0.58 mmol). The resulting mixture was stirred at 70° C. for 30 min. The reaction solvent was removed under vacuum. The crude product was purified by Prep-TLC (DCM:MeOH=30:1) to give the titled compound (50 mg, two step 61% yield). MS: M/e 415 (M+1)⁺.

### Step I: 2-(7-((2S,5R)-2,5-dimethylpiperazin-1-yl)-3,4-dimethyl-5-oxo-4,5-dihydro-3H-imidazo[4,5-b]pyridin-2-yl)acetonitrile

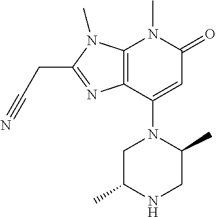 [SMILES: C[C@@H]1CN(c2cc(=O)n(C)c3c2nc(CC#N)n3C)[C@@H](C)CN1]

To a solution of tert-butyl (2R,5S)-4-(2-(cyanomethyl)-3,4-dimethyl-5-oxo-4,5-dihydro-3H-imidazo[4,5-b]pyridin-7-yl)-2,5-dimethylpiperazine-1-carboxylate (50 mg, 0.12 mmol) in DCM (5 mL) were added TFA (1 mL). The resulting mixture was stirred at rt for 3 hours. The reaction solvent was removed under vacuum. The crude product was dissolved with water and adjusted to pH=9 with saturated NaHCO₃. The aqueous layer was extracted with (DCM/IPA=4/1) and purified by Prep-TLC (DCM:MeOH=30:1) to give the titled compound (20 mg, 52%). MS: Me 315 (M+1)⁺.

### Step J: 2-(7-((2S,5R)-2,5-dimethyl-4-(1-(quinoxalin-6-yl)ethyl)piperazin-1-yl)-3,4-dimethyl-5-oxo-4,5-dihydro-3H-imidazo[4,5-b]pyridin-2-yl)acetonitrile

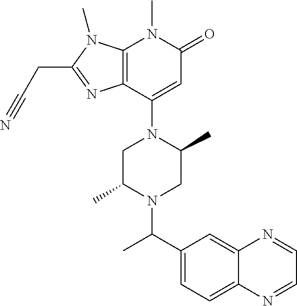 [SMILES: CC(c1ccc2nccnc2c1)N1C[C@H](C)N(c2cc(=O)n(C)c3c2nc(CC#N)n3C)C[C@H]1C]

To a solution of 2-(7-((2S,5R)-2,5-dimethylpiperazin-1-yl)-3,4-dimethyl-5-oxo-4,5-dihydro-3H-imidazo[4,5-b]pyridin-2-yl)acetonitrile (10 mg, 0.032 mmol) in CH₃CN (10 mL) was added 1-(quinoxalin-6-yl)ethan-1-ol (11 mg, 0.064 mmol), (cyanomethyl)trimethylphosphonium iodide (31 mg, 0.127 mmol) and DIPEA (41 mg, 0.32 mmol). The resulting mixture was stirred at 105° C. for 15 hours. The reaction solvent was removed under reduced pressure to dryness. The crude product was purified by Prep-TLC(DCM:MeOH=20:1) and further purified by Prep-HPLC(Method A) to give the titled compound (1 mg, 18%). ¹H NMR (400 MHz, CD₃OD) δ 8.90-8.80 (m, 2H), 8.15-7.95 (m, 3H), 5.52 (s, 1H), 4.20 (s, 2H), 3.97 (s, 3H), 3.83 (s, 3H), 3.75-3.55 (m, 2H), 3.50-3.33 (m, 2H), 3.20-3.02 (m, 1H), 2.98-2.63 (m, 2H), 2.25-2.15 (m, 1H), 1.49-1.30 (m, 4H), 1.25-1.18 (m, 3H), 1.08-1.01 (m, 1H) ppm. MS: M/e 471 (M+1)⁺.

Compound A1 also can be synthesized according to the following synthetic method.

### Compound A1: 2-(7-((2S,5R)-2,5-dimethyl-4-(1-(quinoxalin-6-yl)ethyl)piperazin-1-yl)-3,4-dimethyl-5-oxo-4,5-dihydro-3H-imidazo[4,5-b]pyridin-2-yl)acetonitrile

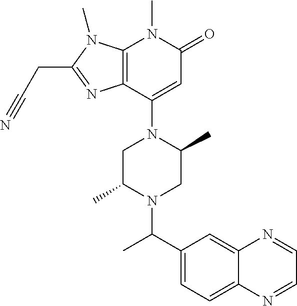 [SMILES: CC(c1ccc2nccnc2c1)N1C[C@H](C)N(c2cc(=O)n(C)c3c2nc(CC#N)n3C)C[C@H]1C]

### Step A: tert-butyl (2R,5S)-4-(5-chloro-2-(hydroxymethyl)-3-methyl-3H-imidazo[4,5-b]pyridin-7-yl)-2,5-dimethylpiperazine-1-carboxylate

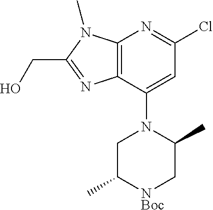 [SMILES: C[C@@H]1CN(c2cc(Cl)nc3c2nc(CO)n3C)[C@@H](C)CN1C(=O)OC(C)(C)C]

To a solution of methyl 7-((2S,5R)-4-(tert-butoxycarbonyl)-2,5-dimethylpiperazin-1-yl)-5-chloro-3-methyl-3H-imidazo[4,5-b]pyridine-2-carboxylate (1.6 g, 3.66 mmol) in THE (10 mL) were added NaBH₄ (278 mg, 7.32 mmol). The resulting mixture was stirred at 70° C. for 24 hours. The reaction solvent was quenched by water and extracted with EtOAc. The residue was purified by flash column chromatography (DCM:MeOH=20:1) to give the titled compound (1.2 g, 80% yield) as a white solid. MS: M/e 410 (M+1)⁺.

### Step B: tert-butyl (2R,5S)-4-(2-(((tert-butyldimethylsilyl)oxy)methyl)-5-chloro-3-methyl-3H-imidazo[4,5-b]pyridin-7-yl)-2,5-dimethylpiperazine-1-carboxylate

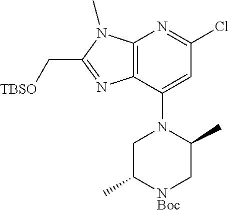 [SMILES: C[C@@H]1CN(c2cc(Cl)nc3c2nc(CO[Si](C)(C)C(C)(C)C)n3C)[C@@H](C)CN1C(=O)OC(C)(C)C]

To a mixture of tert-butyl (2R,5S)-4-(5-chloro-2-(hydroxymethyl)-3-methyl-3H-imidazo[4,5-b]pyridin-7-yl)-2,5-dimethylpiperazine-1-carboxylate (1.1 g, 2.6 mmol) in DCM (15 mL) and was added TBSCl (0.58 g, 3.9 mmol) and imidazole (0.53 g, 7.78 mmol). The resulting mixture was stirred at RT overnight. The reaction solvent was washed with water and purified by flash column chromatography (PE/EtOAc) to give the titled compound (1.2 g, 85% yield) as a colourless oil. MS: M/e 524 (M+1)⁺.

### Step C tert-butyl (2R,5S)-4-(2-(((tert-butyldimethylsilyl)oxy)methyl)-3-methyl-5-oxo-4,5-dihydro-3H-imidazo[4,5-b]pyridin-7-yl)-2,5-dimethylpiperazine-1-carboxylate

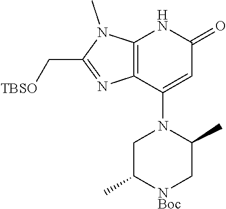 [SMILES: C[C@@H]1CN(c2cc(=O)[nH]c3c2nc(CO[Si](C)(C)C(C)(C)C)n3C)[C@@H](C)CN1C(=O)OC(C)(C)C]

To a solution of tert-butyl (2R,5S)-4-(2-(((tert-butyldimethylsilyl)oxy)methyl)-5-chloro-3-methyl-3H-imidazo[4,5-b]pyridin-7-yl)-2,5-dimethylpiperazine-1-carboxylate (1.2 g, 2.3 mmol) in 1,4-dioxane (20 mL) were added t-BuXphos (98 mg, 0.11 mmol), KOH (385 mg, 6.9 mmol in 2.5 mL water) and Pd₂(dba)₃ (107 mg, 0.11 mmol). The resulting mixture was stirred at 80° C. for 3 hours. The reaction solvent was poured into water and extracted by EtOAc (discard). The aqueous layer was adjusted to pH=3 with citric acid. The aqueous layer was extracted with EtOAc and purified by flash column chromatography (PE/EtOAc) to give the titled compound (900 mg, crude) as a white solid. MS: M/e 506 (M+1)⁺.

### Step D tert-butyl (2R,5S)-4-(2-(((tert-butyldimethylsilyl)oxy)methyl)-3,4-dimethyl-5-oxo-4,5-dihydro-3H-imidazo[4,5-b]pyridin-7-yl)-2,5-dimethylpiperazine-1-carboxylate

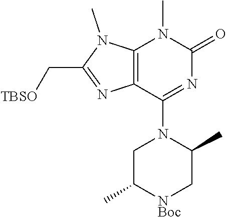 [SMILES: C[C@@H]1CN(c2nc(=O)n(C)c3c2nc(CO[Si](C)(C)C(C)(C)C)n3C)[C@@H](C)CN1C(=O)OC(C)(C)C]

To a mixture of tert-butyl (2R,5S)-4-(2-(((tert-butyldimethylsilyl)oxy)methyl)-3-methyl-5-oxo-4,5-dihydro-3H-imidazo[4,5-b]pyridin-7-yl)-2,5-dimethylpiperazine-1-carboxylate (600 mg, 1.19 mmol) in dioxane (80 mL) and was added Cs₂CO₃ (2.3 g, 7.1 mmol) and CH₃I (1 g, 7.1 mmol). The resulting mixture was stirred at rt overnight. The reaction solvent was removed under reduced pressure to dryness. The crude product was purified by flash column chromatography (PE/EtOAc and DCM/MeOH) to give the titled compound (380 mg). MS: M/e 520 (M+1)⁺.

### Step E: tert-butyl (2R,5S)-4-(2-(hydroxymethyl)-3,4-dimethyl-5-oxo-4,5-dihydro-3H-imidazo[4,5-b]pyridin-7-yl)-2,5-dimethylpiperazine-1-carboxylate

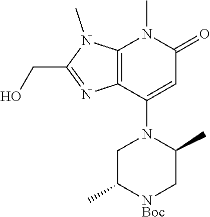 [SMILES: C[C@@H]1CN(c2cc(=O)n(C)c3c2nc(CO)n3C)[C@@H](C)CN1C(=O)OC(C)(C)C]

To a solution of tert-butyl (2R,5S)-4-(2-(((tert-butyldimethylsilyl)oxy)methyl)-3,4-dimethyl-5-oxo-4,5-dihydro-3H-imidazo[4,5-b]pyridin-7-yl)-2,5-dimethylpiperazine-1-carboxylate (380 mg, 0.73 mmol) in THF (10 mL) were added TBAF (1.1 mL, 1.1 mmol). The resulting mixture was stirred at rt for 1 hour. The reaction solvent was concentrated to dryness. The resulting residue was purified by flash column chromatography (DCM/MeOH) to give the titled compound (300 mg, crude) as a yellow solid. MS: M/e 406 (M+1)⁺.

### Step F: tert-butyl (2R,5S)-4-(2-(chloromethyl)-3,4-dimethyl-5-oxo-4,5-dihydro-3H-imidazo[4,5-b]pyridin-7-yl)-2,5-dimethylpiperazine-1-carboxylate

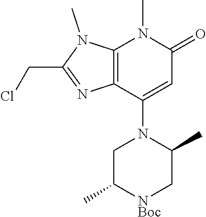 [SMILES: C[C@@H]1CN(c2cc(=O)n(C)c3c2nc(CCl)n3C)[C@@H](C)CN1C(=O)OC(C)(C)C]

To a solution of tert-butyl (2R,5S)-4-(2-(hydroxymethyl)-3,4-dimethyl-5-oxo-4,5-dihydro-3H-imidazo[4,5-b]pyridin-7-yl)-2,5-dimethylpiperazine-1-carboxylate (150 mg, 0.37 mmol) in DCM (150 mL) were added SOCl₂ (220 mg, 1.85 mmol). The resulting mixture was stirred at rt for 5 min. The reaction solvent was washed with water, NaHCO₃ aq. and NaCl aq., The organic layer was concentrated to give the titled compound which was used to next step directly without further purification.

### Step G: tert-butyl (2R,5S)-4-(2-(cyanomethyl)-3,4-dimethyl-5-oxo-4,5-dihydro-3H-imidazo[4,5-b]pyridin-7-yl)-2,5-dimethylpiperazine-1-carboxylate

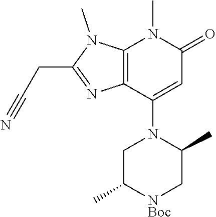 [SMILES: C[C@@H]1CN(c2cc(=O)n(C)c3c2nc(CC#N)n3C)[C@@H](C)CN1C(=O)OC(C)(C)C]

To the solution of tert-butyl (2R,5S)-4-(2-(chloromethyl)-3,4-dimethyl-5-oxo-4,5-dihydro-3H-imidazo[4,5-b]pyridin-7-yl)-2,5-dimethylpiperazine-1-carboxylate (crude) in CH₃CN (20 mL) were added TMSCN (183 mg, 1.85 mmol) and TBAF (1.8 ml, 1.85 mmol). The resulting mixture was stirred at 0° C. for 30 min. The reaction solvent was removed under vacuum. The crude product was purified by Prep-TLC (DCM:MeOH=30:1) to give the titled compound (60 mg). MS: M/e 415 (M+1)⁺.

### Step H: 2-(7-((2S,5R)-2,5-dimethylpiperazin-1-yl)-3,4-dimethyl-5-oxo-4,5-dihydro-3H-imidazo[4,5-b]pyridin-2-yl)acetonitrile

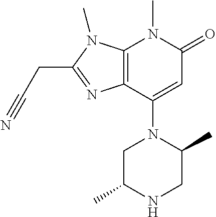 [SMILES: C[C@@H]1CN(c2cc(=O)n(C)c3c2nc(CC#N)n3C)[C@@H](C)CN1]

To a solution of tert-butyl (2R,5S)-4-(2-(cyanomethyl)-3,4-dimethyl-5-oxo-4,5-dihydro-3H-imidazo[4,5-b]pyridin-7-yl)-2,5-dimethylpiperazine-1-carboxylate (120 mg, 0.29 mmol) in DCM (10 mL) were added TFA (3 mL). The resulting mixture was stirred at rt for 1.5 hours. The reaction solvent was removed under vacuum. The crude product was dissolved with water and adjusted to pH=9 with NaHCO₃ aq. The aqueous layer was extracted with (DCM:IPA=3:1) to give the titled compound (100 mg, crude) as a yellow solid. MS: M/e 315 (M+1)⁺.

### Step I: 2-(7-((2S,5R)-2,5-dimethyl-4-(1-(quinoxalin-6-yl)ethyl)piperazin-1-yl)-3,4-dimethyl-5-oxo-4,5-dihydro-3H-imidazo[4,5-b]pyridin-2-yl)acetonitrile

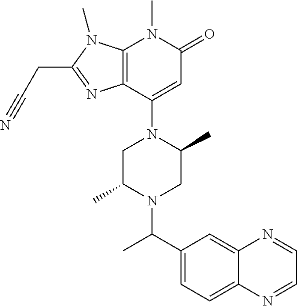 [SMILES: CC(c1ccc2nccnc2c1)N1C[C@H](C)N(c2cc(=O)n(C)c3c2nc(CC#N)n3C)C[C@H]1C]

To a mixture of 2-(7-((2S,5R)-2,5-dimethylpiperazin-1-yl)-3,4-dimethyl-5-oxo-4,5-dihydro-3H-imidazo[4,5-b]pyridin-2-yl)acetonitrile (100 mg, 0.32 mmol) in CH₃CN (10 mL) and was added 1-(quinoxalin-6-yl)ethan-1-ol (83 mg, 0.48 mmol), (cyanomethyl)trimethylphosphonium iodide (387 mg, 1.6 mmol) and DIPEA (410 mg, 3.2 mmol). The resulting mixture was stirred at 105° C. overnight. The reaction solvent was removed under reduced pressure to dryness. The crude product was purified by Prep-TLC (DCM:MeOH=50:1) to give the titled Compound A1 which was further separated into Compound A1a (1 mg) and Compound A1b (1 mg) by Prep-HPLC(Method B).

Compound A1a (the earlier peak): ¹H NMR (400 MHz, CD₃OD) δ 8.90-8.8. (m, 2H), 8.20-7.98 (m, 3H), 5.52 (s, 1H), 5.09-4.95 (m, 1H), 4.55-4.30 (m, 1H), 4.00-3.90 (m, 4H), 3.83 (s, 3H), 3.50-3.34 (m, 1H), 3.10-3.02 (m, 1H), 2.98-2.63 (m, 4H), 1.49-1.30 (m, 6H), 1.08-1.01 (m, 3H) ppm. MS: M/e 471 (M+1)⁺

Compound A1b (the later peak) ¹H NMR (400 MHz, CD₃OD) δ 8.90-8.8. (m, 2H), 8.15-7.95 (m, 3H), 5.52 (s, 2H), 4.87-4.55 (m, 2H), 3.97 (s, 3H), 3.83-3.75 (m, 4H), 3.70-3.55 (m, 2H), 2.95-2.63 (m, 2H), 2.25-2.15 (m, 1H), 1.49-1.30 (m, 3H), 1.25-1.13 (m, 6H) ppm. MS: M/e 471 (M+1)⁺

### Compound A4: 2-(7-((2S,5R)-4-(1-(4-fluoro-2-methoxyphenyl)ethyl)-2,5-dimethylpiperazin-1-yl)-3,4-dimethyl-5-oxo-4,5-dihydro-3H-imidazo[4,5-b]pyridin-2-yl)acetonitrile

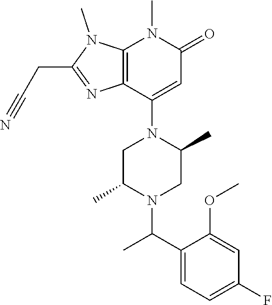 [SMILES: COc1cc(F)ccc1C(C)N1C[C@H](C)N(c2cc(=O)n(C)c3c2nc(CC#N)n3C)C[C@H]1C]

To a solution of 2-(7-((2S,5R)-2,5-dimethylpiperazin-1-yl)-3,4-dimethyl-5-oxo-4,5-dihydro-3H-imidazo[4,5-b]pyridin-2-yl)acetonitrile (30 mg, 0.096 mmol) in CH₃CN (3 mL) was added 1-(4-fluoro-2-methoxyphenyl)ethan-1-ol (33 mg, 1.91 mmol), (cyanomethyl)trimethylphosphonium iodide (116 mg, 0.48 mmol) and DIPEA (123 mg, 0.96 mmol). The resulting mixture was stirred at 105° C. overnight. The reaction solvent was removed under reduced pressure to dryness. The crude product was purified by Prep-TLC (DCM:MeOH=20:1) to give the titled Compound A4, which was further separated into Compound A4a (0.7 mg) and Compound A4b (0.91 mg) by Prep-HPLC (Method A).

Compound A4a (the earlier peak): ¹H NMR (400 MHz, CD₃OD) δ 7.58-7.50 (m, 1H), 6.78-6.59 (m, 2H), 5.51 (s, 1H), 4.80-4.55 (m, 1H), 4.55-3.98 (m, 2H), 3.97 (s, 3H), 3.83 (s, 6H), 3.60-3.50 (m, 1H), 2.98-2.55 (m, 4H), 2.25-2.10 (m, 1H), 1.35-1.01 (m, 9H) ppm. MS: M/e 467 (M+1)⁺.

Compound A4b (the later peak): ¹H NMR (400 MHz, CD₃OD) δ 7.55-7.45 (m, 1H), 6.68-6.56 (m, 2H), 5.51 (s, 1H), 4.57-4.10 (m, 2H), 4.05-3.85 (m, 4H), 3.85-3.55 (m, 5H), 3.50-3.34 (m, 2H), 2.99-2.60 (m, 3H), 2.15-1.98 (m, 1H), 1.60-1.10 (m, 5H), 1.08-0.60 (m, 4H) ppm. MS: M/e 467 (M+1)⁺.

### Compound A5: 2-(7-((2S,5R)-2,5-dimethyl-4-(1-(p-tolyl)ethyl)piperazin-1-yl)-3,4-dimethyl-5-oxo-4,5-dihydro-3H-imidazo[4,5-b]pyridin-2-yl)acetonitrile

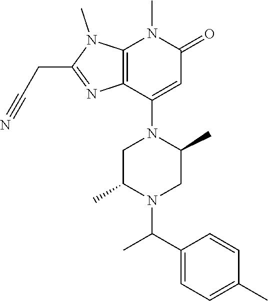 [SMILES: Cc1ccc(C(C)N2C[C@H](C)N(c3cc(=O)n(C)c4c3nc(CC#N)n4C)C[C@H]2C)cc1]

To a mixture of 2-(7-((2S,5R)-2,5-dimethylpiperazin-1-yl)-3,4-dimethyl-5-oxo-4,5-dihydro-3H-imidazo[4,5-b]pyridin-2-yl)acetonitrile (30 mg, 0.1 mmol) in CH₃CN (3 mL) and was added 1-(p-tolyl)ethan-1-ol (39 mg, 0.29 mmol), (cyanomethyl)trimethylphosphonium iodide (116 mg, 0.5 mmol) and DIPEA (123 mg, 1 mmol). The resulting mixture was stirred at 105° C. overnight. The reaction solvent was removed under reduced pressure to dryness. The crude product was purified by Prep-TLC (DCM:MeOH=20:1) to give the titled Compound A5, which was further separated into Compound A5a (0.65 mg) and Compound A5b (0.48 mg) by Prep-HPLC (Method A).

Compound A5a (the earlier peak): ¹H NMR (400 MHz, CD₃OD) δ 7.25 (d, J=7.9 Hz, 2H), 7.11 (d, J=7.8 Hz, 2H), 5.51 (s, 1H), 4.78-4.65 (m, 1H), 4.21 (s, 1H), 3.97 (s, 3H), 3.97 (s, 3H), 3.66-3.41 (m, 2H), 2.80-2.61 (m, 4H), 2.31 (s, 3H), 2.26-2.19 (m, 1H), 1.34 (d, J=10.5 Hz, 3H), 1.18 (d, J=6.6 Hz, 3H), 1.13 (d, J=6.1 Hz, 3H) ppm. MS: M/e 433 (M+1)⁺.

Compound A5b (the later peak): ¹H NMR (400 MHz, CD₃OD) δ 7.26 (d, J=7.9 Hz, 2H), 7.14 (d, J=7.8 Hz, 2H), 5.50 (s, 1H), 4.56-4.45 (m, 1H), 4.20 (s, 1H), 3.97 (s, 3H), 3.83 (s, 3H), 3.62 (s, 1H), 3.40-3.33 (m, 1H), 2.98-2.75 (m, 5H), 2.32 (s, 3H), 1.36 (d, J=6.5 Hz, 3H), 1.31 (d, J=6.5 Hz, 3H), 0.97 (d, J=6.5 Hz, 3H) ppm. MS: M/e 433 (M+1)⁺.

### Compound A6: 2-(7-((2S,5R)-2,5-dimethyl-4-(1-(1-methyl-5-(trifluoromethyl)-1H-pyrazol-4-yl)ethyl)piperazin-1-yl)-3,4-dimethyl-5-oxo-4,5-dihydro-3H-imidazo[4,5-b]pyridin-2-yl)acetonitrile

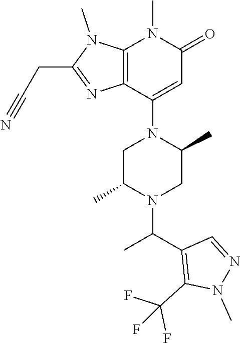 [SMILES: CC(c1cnn(C)c1C(F)(F)F)N1C[C@H](C)N(c2cc(=O)n(C)c3c2nc(CC#N)n3C)C[C@H]1C]

### Step A: 1-(1-methyl-5-(trifluoromethyl)-1H-pyrazol-4-yl)ethan-1-ol

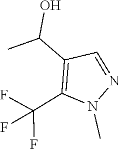

To a solution of 1-methyl-5-(trifluoromethyl)-1H-pyrazole-4-carbaldehyde (200 mg, 1.12 mmol) in THE (8 ml) at −60° C. under N₂, was added MeMgBr (3M, 0.41 ml, 1.23 mmol) dropwise. The solution was warmed to RT naturally and stirred at RT overnight. After completed, the solution was quenched with H₂O (10 ml) and then extracted with EtOAc (10 ml×2). The organic layer was washed with aq. NaHCO₃ (10 ml), brine (10 ml), dried over anhydrous Na₂SO₄ and then concentrated under reduced pressure to give the titled compound (217 mg, 100%), which was used directly for the next step without further purification. MS: M/e 195 (M+1)⁺.

### Step B: 2-(7-((2S,5R)-2,5-dimethyl-4-(1-(1-methyl-5-(trifluoromethyl)-1H-pyrazol-4-yl)ethyl)piperazin-1-yl)-3,4-dimethyl-5-oxo-4,5-dihydro-3H-imidazo[4,5-b]pyridin-2-yl)acetonitrile

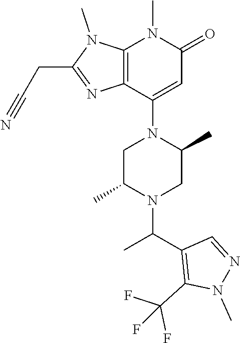 [SMILES: CC(c1cnn(C)c1C(F)(F)F)N1C[C@H](C)N(c2cc(=O)n(C)c3c2nc(CC#N)n3C)C[C@H]1C]

To a mixture of 2-(7-((2S,5R)-2,5-dimethylpiperazin-1-yl)-3,4-dimethyl-5-oxo-4,5-dihydro-3H-imidazo[4,5-b]pyridin-2-yl)acetonitrile (30 mg, 0.1 mmol) in CH₃CN (3 mL) and was added 1-(1-methyl-5-(trifluoromethyl)-1H-pyrazol-4-yl)ethan-1-ol (56 mg, 0.29 mmol), (cyanomethyl)trimethylphosphonium iodide (116 mg, 0.5 mmol) and DIPEA (123 mg, 1 mmol). The resulting mixture was stirred at 105° C. overnight. The reaction solvent was removed under reduced pressure to dryness. The crude product was purified by Prep-TLC (DCM:MeOH=15:1) to give the titled Compound A6, which was further separated into Compound A6a (0.77 mg) and Compound A6b (0.64 mg) by Prep-HPLC (Method A).

Compound A6a (the earlier peak): ¹H NMR (400 MHz, CD₃OD) δ 7.62 (s, 1H), 5.51 (s, 1H), 4.77-4.63 (m, 1H), 4.21 (s, 1H), 3.97 (s, 6H), 3.83 (s, 3H), 3.81-3.70 (m, 1H), 3.57-3.45 (m, 2H), 2.86-2.70 (m, 3H), 2.31 (d, J=12.5 Hz, 1H), 1.35 (d, J=6.6 Hz, 3H), 1.21 (d, J=6.6 Hz, 3H), 1.09 (d, J=6.5 Hz, 3H) ppm. MS: M/e 491 (M+1)⁺.

Compound A6b (the later peak): ¹H NMR (400 MHz, CD₃OD) δ 7.61 (s, 1H), 5.51 (s, 1H), 4.75-4.63 (m, 1H), 4.23-4.17 (m, 1H), 3.98 (s, 3H), 3.97 (s, 3H), 3.83 (s, 3H), 3.39-3.33 (m, 2H), 3.04-2.90 (m, 2H), 2.85-2.61 (m, 3H), 1.33 (d, J=6.7 Hz, 3H), 1.29 (d, J=6.5 Hz, 3H), 1.00 (d, J=6.5 Hz, 3H) ppm. MS: M/e 491 (M+1)⁺.

### Compound A7: 2-(7-((2S,5R)-2,5-dimethyl-4-(1-(2-(trifluoromethyl)pyridin-3-yl)ethyl)piperazin-1-yl)-3,4-dimethyl-5-oxo-4,5-dihydro-3H-imidazo[4,5-b]pyridin-2-yl)acetonitrile

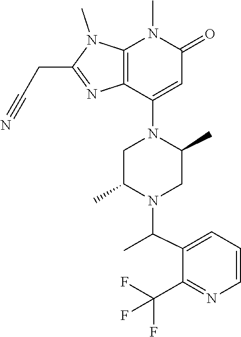 [SMILES: CC(c1cccnc1C(F)(F)F)N1C[C@H](C)N(c2cc(=O)n(C)c3c2nc(CC#N)n3C)C[C@H]1C]

### Step A: 1-(2-(trifluoromethyl)pyridin-3-yl)ethan-1-ol

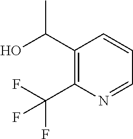

To a solution of 2-(trifluoromethyl)nicotinaldehyde (200 mg, 1.1 mmol) in THE (10 mL) was added methylmagnesium bromide (0.7 mL, 2.2 mmol, 3M in Et₂O) at 0° C. under N₂ atmosphere. The reaction mixture was stirred at room temperature for 2 hours. The reaction was quenched by water (5 mL) and extracted with EtOAc (15 mL×3). The organic phases were concentrated under reduced pressure. The resulting residue was purified by Prep-TLC (PE:EtOAc=2:1) to give the target compound (120 mg, 55% yield) as a clear oil. MS: M/e 192 (M+1)⁺.

### Step B: 2-(7-((2S,5R)-2,5-dimethyl-4-(1-(2-(trifluoromethyl)pyridin-3-yl)ethyl)piperazin-1-yl)-3,4-dimethyl-5-oxo-4,5-dihydro-3H-imidazo[4,5-b]pyridin-2-yl)acetonitrile

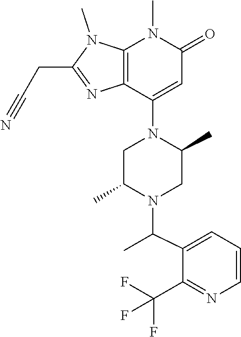 [SMILES: CC(c1cccnc1C(F)(F)F)N1C[C@H](C)N(c2cc(=O)n(C)c3c2nc(CC#N)n3C)C[C@H]1C]

To a mixture of 2-(7-((2S,5R)-2,5-dimethylpiperazin-1-yl)-3,4-dimethyl-5-oxo-4,5-dihydro-3H-imidazo[4,5-b]pyridin-2-yl)acetonitrile (30 mg, 0.1 mmol) in CH₃CN (3 mL) and was added 1-(2-(trifluoromethyl)pyridin-3-yl)ethan-1-ol (56 mg, 0.29 mmol), (cyanomethyl)trimethylphosphonium iodide (116 mg, 0.5 mmol) and DIPEA (123 mg, 1 mmol). The resulting mixture was stirred at 105° C. overnight. The reaction solvent was removed under reduced pressure. The resulting residue was purified by Prep-TLC (DCM:MeOH=20:1) to give the titled Compound A7, which was further separated into Compound A7a (1.21 mg) and Compound A7b (0.81 mg) by Prep-HPLC (Method A).

Compound A7a (the earlier peak): ¹H NMR (400 MHz, CD₃OD) δ 8.55 (d, J=4.6 Hz, 1H), 8.48 (d, J=8.4 Hz, 1H), 7.68 (dd, J=7.9, 4.7 Hz, 1H), 5.51 (s, 1H), 4.20 (d, J=4.9 Hz, 1H), 3.99-3.91 (m, 4H), 3.83 (s, 3H), 3.72-3.53 (m, 3H), 2.85-2.55 (m, 3H), 1.93 (d, J=12.1 Hz, 1H), 1.36 (d, J=6.4 Hz, 3H), 1.21-1.11 (m, 6H) ppm. MS: M/e 488 (M+1)⁺.

Compound A7b (the later peak): ¹H NMR (400 MHz, CD₃OD) δ 8.56 (d, J=4.2 Hz, 1H), 8.51 (d, J=7.9 Hz, 1H), 7.70 (dd, J=8.2, 4.4 Hz, 1H), 5.51 (s, 1H), 4.25-4.16 (m, 2H), 3.97 (s, 3H), 3.83 (s, 3H), 3.42-3.33 (m, 2H), 3.08-2.60 (m, 5H), 1.43 (d, J=6.6 Hz, 3H), 1.29 (d, J=6.4 Hz, 3H), 1.02 (d, J=6.5 Hz, 3H) ppm. MS: M/e 488 (M+1)⁺.

### Compound A9: 2-(7-((2S,5R)-4-(1-(4-fluoro-2-(trifluoromethyl)phenyl)ethyl)-2,5-dimethyl piperazin-1-yl)-4-methyl-5-oxo-4,5-dihydro-3H-imidazo[4,5-b]pyridin-2-yl)acetonitrile

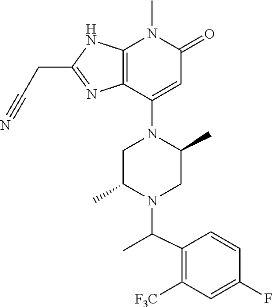 [SMILES: CC(c1ccc(F)cc1C(F)(F)F)N1C[C@H](C)N(c2cc(=O)n(C)c3[nH]c(CC#N)nc23)C[C@H]1C]

### Step A: tert-butyl (tert-butoxycarbonyl)(4,6-dichloro-3-nitropyridin-2-yl)carbamate

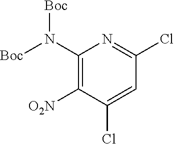 [SMILES: CC(C)(C)OC(=O)N(C(=O)OC(C)(C)C)c1nc(Cl)cc(Cl)c1[N+](=O)[O-]]

To a solution of 4,6-dichloro-3-nitropyridin-2-amine (2 g, 9.66 mmol) in THE (20 ml) at 0° C. was added NaH (60%, 1.16 g, 29 mmol). Then (Boc)₂O (6.3 g, 28.90 mmol) was added. The mixture was stirred at rt overnight. The reaction was diluted with EtOAc (30 ml) and then washed with water (20 ml). The organic layer was concentrated under reduced pressure. The resulting residue was purified by flash column chromatography with 0-60% EtOAc in PE to give the titled compound (3.63 g, 92%). MS: M/e 408 (M+1)⁺.

### Step B: tert-butyl (tert-butoxycarbonyl)(6-chloro-4-((2S,5R)-4-(1-(4-fluoro-2-(trifluoromethyl)phenyl)ethyl)-2,5-dimethylpiperazin-1-yl)-3-nitropyridin-2-yl)carbamate

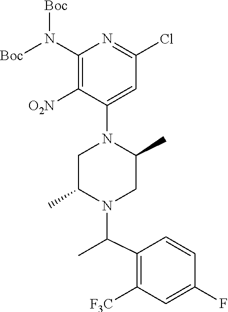 [SMILES: CC(c1ccc(F)cc1C(F)(F)F)N1C[C@H](C)N(c2cc(Cl)nc(N(C(=O)OC(C)(C)C)C(=O)OC(C)(C)C)c2[N+](=O)[O-])C[C@H]1C]

A solution of tert-butyl (tert-butoxycarbonyl)(4,6-dichloro-3-nitropyridin-2-yl)carbamate (1.9 g, 4.67 mmol), (2R,5S)-1-(1-(4-fluoro-2-(trifluoromethyl)phenyl)ethyl)-2,5-dimethylpiperazine (1.5 g, 4.93 mmol) and DIPEA (3 g, 23.26 mmol) in DCM (30 ml) was stirred at RT overnight. The mixture was concentrated under reduced pressure. The resulting residue was purified by flash column chromatography with 0-15% EtOAc in PE to give the titled compound (1.8 g, 57%). MS: M/e 676 (M+1)⁺.

### Step C: tert-butyl (tert-butoxycarbonyl)(4-((2S,5R)-4-(1-(4-fluoro-2-(trifluoromethyl)phenyl)ethyl)-2,5-dimethylpiperazin-1-yl)-3-nitro-6-oxo-1,6-dihydropyridin-2-yl)carbamate

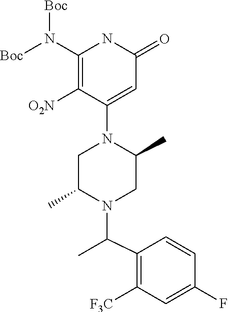 [SMILES: CC(c1ccc(F)cc1C(F)(F)F)N1C[C@H](C)N(c2cc(=O)[nH]c(N(C(=O)OC(C)(C)C)C(=O)OC(C)(C)C)c2[N+](=O)[O-])C[C@H]1C]

A solution of tert-butyl (tert-butoxycarbonyl)(6-chloro-4-((2S,5R)-4-(1-(4-fluoro-2-(trifluoromethyl)phenyl)ethyl)-2,5-dimethylpiperazin-1-yl)-3-nitropyridin-2-yl)carbamate (0.6 g, 0.89 mmol), Pd₂(dba)₃ (81.4 mg, 0.089 mmol), t-BuXPhos (75.6 mg, 0.18 mmol) and KOH (100 mg, 1.79 mmol) in dioxane (15 ml) and water (3 ml) was stirred at 80° C. for 3 h in a sealed tube under N₂. The mixture was concentrated. The residue was diluted with EtOAc (20 ml) and washed with brine (10 ml). The organic layer was concentrated to dryness. The resulting residue was purified by flash column chromatography with 0-60% EtOAc in PE to give the titled compound (0.4 g, 68%). MS: M/e 658 (M+1)⁺.

### Step D: tert-butyl (tert-butoxycarbonyl)(4-((2S,5R)-4-(1-(4-fluoro-2-(trifluoromethyl)phenyl)ethyl)-2,5-dimethylpiperazin-1-yl)-1-methyl-3-nitro-6-oxo-1,6-dihydropyridin-2-yl)carbamate

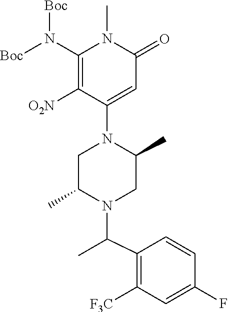 [SMILES: CC(c1ccc(F)cc1C(F)(F)F)N1C[C@H](C)N(c2cc(=O)n(C)c(N(C(=O)OC(C)(C)C)C(=O)OC(C)(C)C)c2[N+](=O)[O-])C[C@H]1C]

A solution of tert-butyl (tert-butoxycarbonyl)(4-((2S,5R)-4-(1-(4-fluoro-2-(trifluoromethyl)phenyl)ethyl)-2,5-dimethylpiperazin-1-yl)-3-nitro-6-oxo-1,6-dihydropyridin-2-yl)carbamate (1.2 g, 1.83 mmol), Mel (0.52 g, 3.66 mmol) and Cs₂CO₃ (1.8 g, 5.52 mmol) in dioxane (20 ml) was stirred at 80° C. overnight. The mixture was concentrated. The residue was diluted with EtOAc (30 ml) and washed with brine (15 ml). The organic layer was concentrated to dryness. The resulting residue was purified by flash column chromatography with 0-50% EtOAc in PE to give the titled compound (0.66 g, 54%). MS: M/e 672 (M+1)⁺.

### Step E: tert-butyl (3-amino-4-((2S,5R)-4-(1-(4-fluoro-2-(trifluoromethyl)phenyl)ethyl)-2,5-dimethylpiperazin-1-yl)-1-methyl-6-oxo-1,6-dihydropyridin-2-yl)(tert-butoxycarbonyl)carbamate

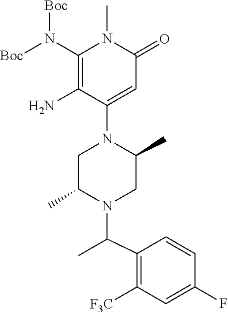 [SMILES: CC(c1ccc(F)cc1C(F)(F)F)N1C[C@H](C)N(c2cc(=O)n(C)c(N(C(=O)OC(C)(C)C)C(=O)OC(C)(C)C)c2N)C[C@H]1C]

A solution of tert-butyl (tert-butoxycarbonyl)(4-((2S,5R)-4-(1-(4-fluoro-2-(trifluoromethyl)phenyl)ethyl)-2,5-dimethylpiperazin-1-yl)-1-methyl-3-nitro-6-oxo-1,6-dihydropyridin-2-yl)carbamate (0.66 g, 0.98 mmol) and Pd/C (0.1 g) in MeOH (15 ml) under H₂ was stirred at rt overnight. The mixture was filtered. The filtrate was concentrated to dryness to give the titled compound (0.62 g, 98%). MS: M/e 642 (M+1)⁺.

### Step F: 5,6-diamino-4-((2S,5R)-4-(1-(4-fluoro-2-(trifluoromethyl)phenyl)ethyl)-2,5-dimethylpiperazin-1-yl)-1-methylpyridin-2 (1H)-one

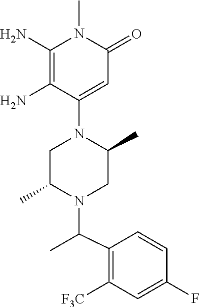 [SMILES: CC(c1ccc(F)cc1C(F)(F)F)N1C[C@H](C)N(c2cc(=O)n(C)c(N)c2N)C[C@H]1C]

A solution of tert-butyl (3-amino-4-((2S,5R)-4-(1-(4-fluoro-2-(trifluoromethyl)phenyl)ethyl)-2,5-dimethylpiperazin-1-yl)-1-methyl-6-oxo-1,6-dihydropyridin-2-yl)(tert-butoxycarbonyl)carbamate (310 mg, 0.48 mmol) and TFA (3 ml) in DCM (15 ml) was stirred at rt for 2 h. The mixture was washed with NaHCO₃ (aq., 10 ml), brine (10 ml), dried over Na₂SO₄ and then concentrated to dryness to give the titled compound (200 mg, 93%). MS: M/e 442 (M+1)⁺.

### Step G: N-(3-amino-4-((2S,5R)-4-(1-(4-fluoro-2-(trifluoromethyl)phenyl)ethyl)-2,5-dimethylpiperazin-1-yl)-1-methyl-6-oxo-1,6-dihydropyridin-2-yl)-2-cyanoacetamide

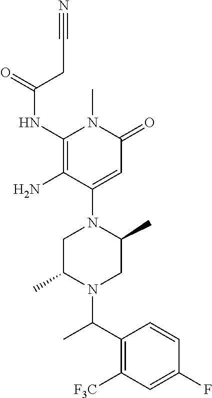 [SMILES: CC(c1ccc(F)cc1C(F)(F)F)N1C[C@H](C)N(c2cc(=O)n(C)c(NC(=O)CC#N)c2N)C[C@H]1C]

A solution of 5,6-diamino-4-((2S,5R)-4-(1-(4-fluoro-2-(trifluoromethyl)phenyl)ethyl)-2,5-dimethylpiperazin-1-yl)-1-methylpyridin-2 (1H)-one (200 mg, 0.45 mmol), 2-cyanoacetic acid (46 mg, 0.54 mmol), HATU (259 mg, 0.68 mmol) and DIPEA (176 mg, 1.36 mmol) in THE (10 ml) was stirred at rt for 30 min. The mixture was diluted with EtOAc (20 ml) and washed with brine (15 ml). The organic layer was concentrated. The resulting residue was purified by flash column chromatography with 0-10% MeOH in DCM to give the titled compound (130 mg, 56%). MS: M/e 509 (M+1)⁺.

### Step H: 2-(7-((2S,5R)-4-(1-(4-fluoro-2-(trifluoromethyl)phenyl)ethyl)-2,5-dimethylpiperazin-1-yl)-4-methyl-5-oxo-4,5-dihydro-3H-imidazo[4,5-b]pyridin-2-yl)acetonitrile

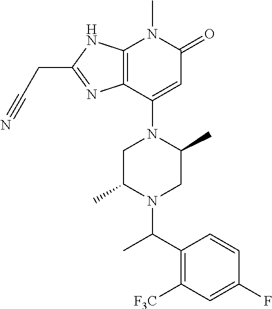 [SMILES: CC(c1ccc(F)cc1C(F)(F)F)N1C[C@H](C)N(c2cc(=O)n(C)c3[nH]c(CC#N)nc23)C[C@H]1C]

A solution of N-(3-amino-4-((2S,5R)-4-(1-(4-fluoro-2-(trifluoromethyl)phenyl)ethyl)-2,5-dimethylpiperazin-1-yl)-1-methyl-6-oxo-1,6-dihydropyridin-2-yl)-2-cyanoacetamide (65 mg, 0.13 mmol) in AcOH (1 ml) was stirred at 80° C. overnight. The mixture was concentrated under reduced pressure. The resulting residue was purified by Prep-TLC (DCM:MeOH=15:1) to give the titled Compound A9 (18 mg), which was further separated into Compound A9a (4 mg) and Compound A9b (7 mg) by Prep-HPLC (Method A).

Compound A9: ¹H NMR (400 MHz, DMSO-d6) δ 13.10-12.50 (m, 1H), 8.09-7.95 (m, 1H), 7.67-7.49 (m, 2H), 5.44-5.17 (m, 1H), 4.27 (s, 0.5H), 4.23 (s, 0.5H), 4.22-4.11 (m, 1H), 4.01 (s, 0.5H), 3.92-3.72 (m, 1H), 3.57 (s, 0.5H), 3.48-3.35 (m, 4H), 3.26-3.14 (m, 0.5H), 3.13-2.96 (m, 1H), 2.90 (s, 0.5H), 2.85 (d, J=11.4 Hz, 1H), 2.67 (s, 0.5H), 1.87 (d, J=11.5 Hz, 0.5H), 1.28 (d, J=6.2 Hz, 2H), 1.21 (dd, J=11.4, 6.5 Hz, 2.5H), 1.11-1.00 (m, 2H), 0.96-0.85 (m, 2.5H) ppm. MS: M/e 491 (M+1)⁺.

Compound A9a (the earlier peak): ¹H NMR (400 MHz, DMSO-d6)) δ 13.21-12.27 (m, 1H), 8.04-7.95 (m, 1H), 7.63-7.50 (m, 2H), 5.32 (s, 1H), 4.19 (s, 2H), 3.82 (s, 1H), 3.58 (s, 1H), 3.47-3.43 (m, 1H), 3.41 (s, 3H), 3.31-3.25 (m, 1H), 3.10-2.90 (m, 1H), 2.81 (s, 1H), 1.87 (d, J=11.2 Hz, 1H), 1.28 (d, J=6.4 Hz, 3H), 1.07 (d, J=6.4 Hz, 3H), 0.94 (s, 3H) ppm. MS: M/e 491 (M+1)⁺.

Compound A9b (the later peak): ¹H NMR (400 MHz, DMSO-d6)) δ 12.76 (s, 1H), 8.11-8.01 (m, 1H), 7.67-7.52 (m, 2H), 5.26 (s, 1H), 4.22 (s, 2H), 4.00 (s, 1H), 3.41 (s, 3H), 3.31-3.28 (m, 2H), 3.20 (d, J=9.2 Hz, 1H), 3.00 (s, 1H), 2.85 (d, J=11.0 Hz, 1H), 2.69 (s, 1H), 1.22 (t, J=7.6 Hz, 6H), 0.93 (d, J=6.4 Hz, 3H) ppm. MS: M/e 491 (M+1)⁺.

### Compound A10: 2-(6-((2R,5R)-4-(1-(4-fluoro-2-(trifluoromethyl)phenyl)ethyl)-2-(methoxymethyl)-5-methylpiperazin-1-yl)-3,9-dimethyl-2-oxo-3,9-dihydro-2H-purin-8-yl)acetonitrile

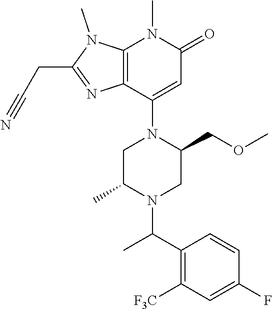 [SMILES: COC[C@H]1CN(C(C)c2ccc(F)cc2C(F)(F)F)[C@H](C)CN1c1cc(=O)n(C)c2c1nc(CC#N)n2C]

### Step A: 2,6-dichloro-9-methyl-9H-purine

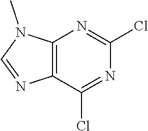

To a solution of 2,6-dichloro-9H-purine (20 g, 106 mmol) and K₂CO₃ (29.3 g, 212 mmol) in DMF (120 mL) was added CH₃I (22.5 g, 158 mmol). The reaction was stirred at room temperature overnight. The reaction mixture was diluted with water, extracted with EtOAc (300 mL×2), washed with brine, dried over Na₂SO₄, filtered, and concentrated to dryness. The resulting residue was purified by flash column chromatography (EtOAc:PE=1:1) to give the titled compound (14 g, 65%). MS: M/e 203 (M+1)⁺.

### Step B: tert-butyl (2R,5R)-4-(2-chloro-9-methyl-9H-purin-6-yl)-5-(hydroxymethyl)-2-methylpiperazine-1-carboxylate

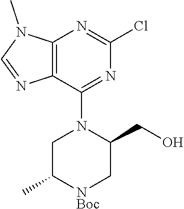 [SMILES: C[C@@H]1CN(c2nc(Cl)nc3c2ncn3C)[C@@H](CO)CN1C(=O)OC(C)(C)C]

To a solution of 2,6-dichloro-9-methyl-9H-purine (2.0 g, 9.9 mmol) and tert-butyl (2R,5R)-5-(hydroxymethyl)-2-methylpiperazine-1-carboxylate (2.28 g, 9.9 mmol) in n-BuOH (40 mL) was added DIPEA (3.83 g, 29.7 mmol). The reaction was stirred at room temperature overnight. The reaction mixture was concentrated under reduced pressure to give the title compound (3.3 g, yield: 84%). MS: M/e 397 (M+1)⁺.

### Step C: tert-butyl (2R,5R)-4-(2-chloro-9-methyl-9H-purin-6-yl)-5-(methoxymethyl)-2-methylpiperazine-1-carboxylate

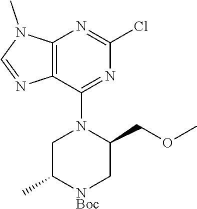 [SMILES: COC[C@H]1CN(C(=O)OC(C)(C)C)[C@H](C)CN1c1nc(Cl)nc2c1ncn2C]

To a solution of tert-butyl (2R,5R)-4-(2-chloro-9-methyl-9H-purin-6-yl)-5-(hydroxymethyl)-2-methylpiperazine-1-carboxylate (3.3 g, 8.329 mmol) in DMF (50 mL) at 0° C. was added NaH (1.33 g, 60% contained, 33.3 mmol). The mixture was stirred at 0° C. for 0.5 hours. Then CH₃I was added. The reaction was stirred at room temperature for 2 hours. The reaction mixture was quenched with water and extracted with EtOAc (120 mL×2). The combined organic layers were washed with brine, dried over Na₂SO₄, filtered and concentrated to dryness. The resulting residue was purified by flash column chromatography (DCM:MeOH=10:1) to give the titled compound (2.4 g, 70%). MS: M/e 411 (M+1)⁺.

### Step D: tert-butyl (2R,5R)-4-(2-hydroxy-9-methyl-9H-purin-6-yl)-5-(methoxymethyl)-2-methylpiperazine-1-carboxylate

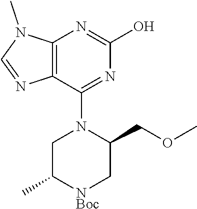 [SMILES: COC[C@H]1CN(C(=O)OC(C)(C)C)[C@H](C)CN1c1nc(O)nc2c1ncn2C]

To a solution of tert-butyl (2R,5R)-4-(2-chloro-9-methyl-9H-purin-6-yl)-5-(methoxymethyl)-2-methylpiperazine-1-carboxylate (2.4 g, 5.85 mmol), Pd₂(dba)₃ (536 mg, 0.585 mmol), t-BuXphos (498 mg, 1.17 mmol) and KOH (5.85 mL, 3M, 17.55 mmol) in dioxane (30 mL) was degassed 3 times under N₂ atmosphere. Then the mixture was stirred at 90° C. for 12 hours. The reaction mixture was diluted with water and extracted with EtOAc (60 mL×2). The combined organic layers were washed with brine, dried over Na₂SO₄, filtered and concentrated to dryness. The resulting residue was purified by flash column chromatography (DCM:MeOH=10:1) to give the titled compound (1.7 g, 74%). MS: M/e 393 (M+1)⁺.

### Step E: tert-butyl (2R,5R)-4-(3,9-dimethyl-2-oxo-3,9-dihydro-2H-purin-6-yl)-5-(methoxymethyl)-2-methylpiperazine-1-carboxylate

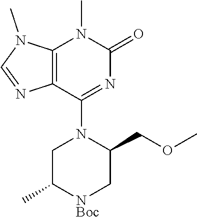 [SMILES: COC[C@H]1CN(C(=O)OC(C)(C)C)[C@H](C)CN1c1nc(=O)n(C)c2c1ncn2C]

To a solution of tert-butyl (2R,5R)-4-(2-hydroxy-9-methyl-9H-purin-6-yl)-5-(methoxymethyl)-2-methylpiperazine-1-carboxylate (1.7 g, 4.33 mmol) and Cs₂CO₃ (2.82 g, 8.66 mmol) in dioxane (40 mL) was added CH₃I (923 mg, 6.50 mmol). The reaction was stirred at room temperature overnight. The reaction mixture was diluted with water, extracted with EtOAc (80 mL×2), washed with brine, dried over Na₂SO₄, filtered, and concentrated to dryness. The resulting residue was purified by flash column chromatography (DCM:MeOH=10:1) to give the titled compound (960 mg, 54%). MS: M/e 407 (M+1)⁺.

### Step F: tert-butyl (2R,5R)-4-(8-bromo-3,9-dimethyl-2-oxo-3,9-dihydro-2H-purin-6-yl)-5-(methoxymethyl)-2-methylpiperazine-1-carboxylate

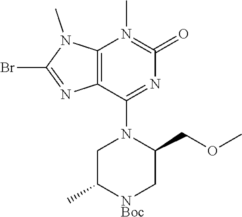

To a solution of tert-butyl (2R,5R)-4-(3,9-dimethyl-2-oxo-3,9-dihydro-2H-purin-6-yl)-5-(methoxymethyl)-2-methylpiperazine-1-carboxylate (300 mg, 0.739 mmol) in CH₃CN (10 mL) was added NBS (197 mg, 1.108 mmol). The reaction was stirred at room temperature for 4 hours. The reaction mixture was diluted with saturated NH₄Cl, extracted with EtOAc (50 mL×2), washed with brine, dried over Na₂SO₄, filtered, and concentrated to dryness. The resulting residue was purified by flash column chromatography (DCM:MeOH=10:1) to give the titled compound (350 mg, 98%). MS: M/e 485 (M+1)⁺.

### Step G: methyl 6-((2R,5R)-4-(tert-butoxycarbonyl)-2-(methoxymethyl)-5-methylpiperazin-1-yl)-3,9-dimethyl-2-oxo-3,9-dihydro-2H-purine-8-carboxylate

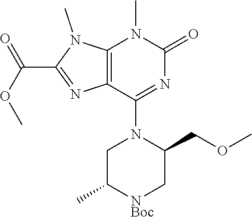 [SMILES: COC[C@H]1CN(C(=O)OC(C)(C)C)[C@H](C)CN1c1nc(=O)n(C)c2c1nc(C(=O)OC)n2C]

To a solution of tert-butyl (2R,5R)-4-(8-bromo-3,9-dimethyl-2-oxo-3,9-dihydro-2H-purin-6-yl)-5-(methoxymethyl)-2-methylpiperazine-1-carboxylate (350 mg, 0.723 mmol), Pd(dppf)Cl₂ and Et₃N (146 mg, 1.446 mmol) in MeOH (10 mL) was degassed 3 times under CO atmosphere. Then the mixture was stirred at 90° C. for 12 hours under CO atmosphere. The reaction mixture was diluted with water and extracted with EtOAc (35 mL×2). The combined organic layers were washed with brine, dried over Na₂SO₄, filtered and concentrated to dryness. The resulting residue was purified by flash column chromatography (DCM:MeOH=8:1) to give the titled compound (280 mg, 83%). MS: M/e 465 (M+1)⁺.

### Step H: tert-butyl (2R,5R)-4-(8-(hydroxymethyl)-3,9-dimethyl-2-oxo-3,9-dihydro-2H-purin-6-yl)-5-(methoxymethyl)-2-methylpiperazine-1-carboxylate

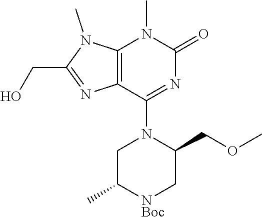 [SMILES: COC[C@H]1CN(C(=O)OC(C)(C)C)[C@H](C)CN1c1nc(=O)n(C)c2c1nc(CO)n2C]

To a solution of methyl 6-((2R,5R)-4-(tert-butoxycarbonyl)-2-(methoxymethyl)-5-methylpiperazin-1-yl)-3,9-dimethyl-2-oxo-3,9-dihydro-2H-purine-8-carboxylate (280 mg, 0.603 mmol) in MeOH (10 mL) was added NaBH₄ (92 mg, 2.414 mmol). The reaction was stirred at room temperature for 2 hours. The reaction mixture was quenched with saturated NH₄Cl, extracted with EtOAc (30 mL×2), washed with brine, dried over Na₂SO₄, filtered, and concentrated to dryness. The resulting residue was purified by flash column chromatography (DCM:MeOH=8:1) to give the titled compound (200 mg, 76%). MS: M/e 437 (M+1)⁺.

### Step I: tert-butyl (2R,5R)-4-(8-(chloromethyl)-3,9-dimethyl-2-oxo-3,9-dihydro-2H-purin-6-yl)-5-(methoxymethyl)-2-methylpiperazine-1-carboxylate

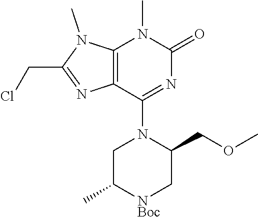 [SMILES: COC[C@H]1CN(C(=O)OC(C)(C)C)[C@H](C)CN1c1nc(=O)n(C)c2c1nc(CCl)n2C]

To a solution of tert-butyl (2R,5R)-4-(8-(hydroxymethyl)-3,9-dimethyl-2-oxo-3,9-dihydro-2H-purin-6-yl)-5-(methoxymethyl)-2-methylpiperazine-1-carboxylate (200 mg, 0.459 mmol) in DCM (8 mL) was added SOCl₂ (218 mg, 1.835 mmol). The reaction was stirred at room temperature for 0.5 hours. The reaction mixture concentrated to give the titled compound (190 mg, 91%). MS: M/e 455 (M+1)⁺.

### Step J: tert-butyl (2R,5R)-4-(8-(cyanomethyl)-3,9-dimethyl-2-oxo-3,9-dihydro-2H-purin-6-yl)-5-(methoxymethyl)-2-methylpiperazine-1-carboxylate

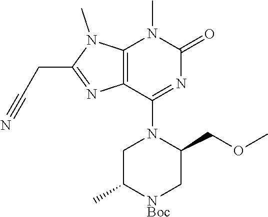 [SMILES: COC[C@H]1CN(C(=O)OC(C)(C)C)[C@H](C)CN1c1nc(=O)n(C)c2c1nc(CC#N)n2C]

To a solution of tert-butyl (2R,5R)-4-(8-(chloromethyl)-3,9-dimethyl-2-oxo-3,9-dihydro-2H-purin-6-yl)-5-(methoxymethyl)-2-methylpiperazine-1-carboxylate (190 mg, 0.419 mmol) in DCM (15 mL) was added TBAF (1.67 mL, 1M, 1.674 mmol), followed TMSCN (166 mg, 1.674 mmol). The reaction was stirred at room temperature overnight. The reaction mixture was diluted with water, extracted with DCM (40 mL×2), washed with brine, dried over Na₂SO₄, filtered, and concentrated to dryness. The resulting residue was purified by flash column chromatography (DCM:MeOH=8:1) to give the titled compound (150 mg, 81%). MS: M/e 446 (M+1)⁺.

### Step K: 2-(6-((2R,5R)-2-(methoxymethyl)-5-methylpiperazin-1-yl)-3,9-dimethyl-2-oxo-3,9-dihydro-2H-purin-8-yl)acetonitrile

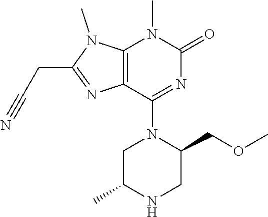 [SMILES: COC[C@H]1CN[C@H](C)CN1c1nc(=O)n(C)c2c1nc(CC#N)n2C]

To a stirred solution of tert-butyl (2R,5R)-4-(8-(cyanomethyl)-3,9-dimethyl-2-oxo-3,9-dihydro-2H-purin-6-yl)-5-(methoxymethyl)-2-methylpiperazine-1-carboxylate (150 mg, 0.337 mmol) in CH₂Cl₂ (15 mL) was added TFA (3 mL). Then reaction mixture was stirred at room temperature for 4 hours, concentrated to give a residue, basified to pH=10˜11 with saturated NaHCO₃ aq., and extracted with CH₂Cl₂/IPA (3/1, 30 mL×3). The combined organic layers were washed with brine, dried over Na₂SO₄, and concentrated to give the titled compound (40 mg, 34%). MS: M/e 346 (M+1)⁺.

### Step L: 2-(6-((2R,5R)-4-(1-(4-fluoro-2-(trifluoromethyl)phenyl)ethyl)-2-(methoxymethyl)-5-methylpiperazin-1-yl)-3,9-dimethyl-2-oxo-3,9-dihydro-2H-purin-8-yl)acetonitrile

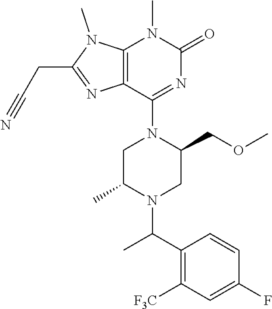 [SMILES: COC[C@H]1CN(C(C)c2ccc(F)cc2C(F)(F)F)[C@H](C)CN1c1nc(=O)n(C)c2c1nc(CC#N)n2C]

A solution of 2-(6-((2R,5R)-2-(methoxymethyl)-5-methylpiperazin-1-yl)-3,9-dimethyl-2-oxo-3,9-dihydro-2H-purin-8-yl)acetonitrile (40 mg, 0.116 mol), 1-(4-fluoro-2-(trifluoromethyl)phenyl)ethan-1-ol (36 mg, 0.174 mol), (cyanomethyl)trimethylphosphonium iodide (56 mg. 0.232 mol) and DIPEA (45 mg, 0.348 mol) in CH₃CN (2 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) to give the titled Compound A10 (6 mg), which was further separated into Compound A10a (2 mg) and Compound A10b (1.5 mg) by Prep-HPLC (Method A).

Compound A10: ¹H NMR (400 MHz, CD₃OD) δ 8.07-7.94 (m, 1H), 7.43-7.39 (m, 2H), 6.32-6.05 (m, 0.5H), 5.95-5.66 (m, 0.5H), 5.37-5.12 (m, 0.5H), 4.81-4.57 (m, 0.5H), 4.21 (dd, J=24.3, 4.1 Hz, 2H), 3.93 (t, J=5.6 Hz, 3H), 3.79 (s, 3H), 3.76-3.59 (m, 3H), 3.43 (d, J=4.0 Hz, 1H), 3.26-3.18 (m, 3H), 2.94-2.53 (m, 2H), 2.35-1.98 (m, 1H), 1.39-1.34 (m, 3H), 1.04-0.89 (m, 3H) ppm. MS: M/e 536 (M+1)⁺

Compound A10a (the earlier peak): ¹H NMR (400 MHz, CD₃OD) δ 8.06-7.91 (m, 1H), 7.45-7.36 (m, 2H), 6.06-6.02 (m, 0.5H), 5.95-5.91 (m, 0.5H), 5.16-5.12 (m, 0.5H), 4.94-4.90 (m, 0.5H), 4.21 (d, J=19.3 Hz, 1H), 3.94 (s, 3H), 3.92-3.88 (m, 1H), 3.79 (d, J=2.7 Hz, 3H), 3.69-3.60 (m, 3H), 3.40-3.33 (m, 1H), 3.22 (d, J=10.8 Hz, 3H), 2.94-2.51 (m, 2H), 2.27 (d, J=12.3 Hz, 1H), 1.31 (d, J=6.2 Hz, 3H), 1.11-0.98 (m, 3H) ppm. MS: M/e 536 (M+1)⁺

Compound A10b (the later peak): ¹H NMR (400 MHz, CD₃OD) δ 8.08-7.98 (m, 1H), 7.44-7.39 (m, 2H), 6.31 (s, 0.5H), 5.67 (d, J=13.6 Hz, 0.5H), 5.39 (s, 0.5H), 4.64-4.57 (m, 0.5H), 4.11-3.98 (m, 2H), 3.93 (d, J=11.3 Hz, 3H), 3.85-3.73 (m, 4H), 3.50-3.45 (m, 0.5H), 3.43 (d, J=3.9 Hz, 3H), 3.35-3.31 (m, 0.5H), 3.27-3.06 (m, 1.5H), 3.01-2.59 (m, 3.5H), 1.29-1.21 (m, 3H), 0.90 (d, J=6.5 Hz, 3H) ppm. MS: M/e 536 (M+1)⁺

### Compound A11: 2-(6-((2R,5R)-4-(1-(2,3-dihydrobenzo[b][1,4]dioxin-6-yl)ethyl)-2-(methoxymethyl)-5-methylpiperazin-1-yl)-3,9-dimethyl-2-oxo-3,9-dihydro-2H-purin-8-yl)acetonitrile

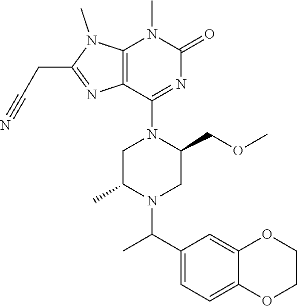 [SMILES: COC[C@H]1CN(C(C)c2ccc3c(c2)OCCO3)[C@H](C)CN1c1nc(=O)n(C)c2c1nc(CC#N)n2C]

### Step A: 1-(2,3-dihydrobenzo[b][1,4]dioxin-6-yl)ethan-1-ol

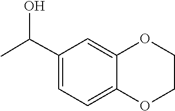

To a solution of 1-(2,3-dihydrobenzo[b][1,4]dioxin-6-yl)ethan-1-one (720 mg, 4.1 mmol) in MeOH (20 mL) was added NaBH₄ (228 mg, 6.0 mmol) at room temperature and the mixture was stirred at room temperature for 2 hours. The reaction mixture was concentrated to dryness. The resulting residue was treated with saturated NaHCO₃ aq., extracted with EtOAc (20 mL×3). The combined layers were washed with brine (20 mL×3), dried over Na₂SO₄ and concentrated to dryness. The resulting residue was purified by flash column chromatography to give the titled compound (360 mg, 50%). MS: M/e 181 (M+1)⁺.

### Step B: 2-(6-((2R,5R)-4-(1-(2,3-dihydrobenzo[b][1,4]dioxin-6-yl)ethyl)-2-(methoxymethyl)-5-methylpiperazin-1-yl)-3,9-dimethyl-2-oxo-3,9-dihydro-2H-purin-8-yl)acetonitrile

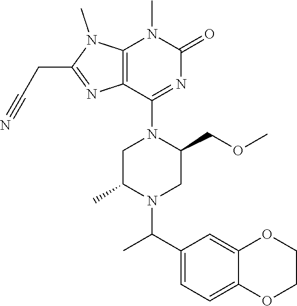 [SMILES: COC[C@H]1CN(C(C)c2ccc3c(c2)OCCO3)[C@H](C)CN1c1nc(=O)n(C)c2c1nc(CC#N)n2C]

A solution of 2-(6-((2R,5R)-2-(methoxymethyl)-5-methylpiperazin-1-yl)-3,9-dimethyl-2-oxo-3,9-dihydro-2H-purin-8-yl)acetonitrile (50 mg, 0.145 mol), 1-(2,3-dihydrobenzo[b][1,4]dioxin-6-yl)ethan-1-ol (52 mg, 0.290 mol), (cyanomethyl)trimethylphosphonium iodide (70 mg. 0.290 mol) and DIPEA (56 mg, 0.435 mol) in CH₃CN (1 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) to give the titled Compound A11, which was further separated into Compound A11a (3 mg) and Compound A11b (4 mg) by Prep-HPLC (Method A).

Compound A11a (the earlier peak): ¹H NMR (400 MHz, CD₃OD) δ 6.85 (s, 1H), 6.81-6.74 (m, 2H), 5.98 (d, J=53.1 Hz, 1H), 5.16 (s, 0.5H), 4.89-4.84 (m, 0.5H), 4.30-4.19 (m, 4.5H), 3.94 (s, 3H), 3.79 (s, 3H), 3.76-3.65 (m, 1.5H), 3.63-3.58 (m, 2H), 3.49-3.40 (m, 1H), 3.36-3.32 (m, 1H), 3.26 (s, 3H), 2.83-2.58 (m, 3H), 1.31 (d, J=6.3 Hz, 3H), 1.04 (d, J=6.3 Hz, 3H) ppm. MS: M/e 508 (M+1)⁺

Compound A11b (the later peak): ¹H NMR (400 MHz, CD₃OD) δ 6.84 (s, 1H), 6.83-6.75 (m, 2H), 6.24 (s, 0.5H), 5.69 (s, 0.5H), 5.32 (s, 0.5H), 4.59 (s, 0.5H), 4.30-4.21 (m, 4.5H), 3.96-3.82 (m, 4H), 3.80-3.75 (m, 4H), 3.70-3.48 (m, 2H), 3.39 (s, 3H), 3.19-3.06 (m, 1.5H), 2.95 (s, 1H), 2.88-2.64 (m, 2H), 1.29 (d, J=6.4 Hz, 3H), 0.88 (d, J=6.5 Hz, 3H) ppm. MS: M/e 508 (M+1)⁺

### Compound A12: 2-(6-((2R,5R)-4-(1-(3,3-dimethyl-2,3-dihydrobenzo[b][1,4]dioxin-6-yl)ethyl)-2-(methoxymethyl)-5-methylpiperazin-1-yl)-3,9-dimethyl-2-oxo-3,9-dihydro-2H-purin-8-yl)acetonitrile

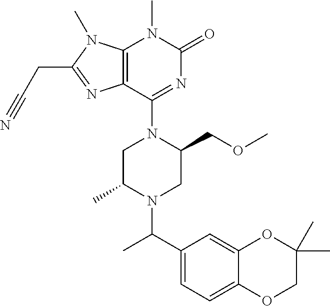 [SMILES: COC[C@H]1CN(C(C)c2ccc3c(c2)OC(C)(C)CO3)[C@H](C)CN1c1nc(=O)n(C)c2c1nc(CC#N)n2C]

### Step A: 3,3-dimethyl-2,3-dihydrobenzo[b][1,4]dioxine-6-carbonitrile

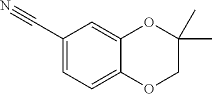

To a solution of 4-fluoro-3-hydroxybenzonitrile (100 g, 0.73 mol) and Cs₂CO₃ (951 g, 2.92 mol) in DMAc (1000 mL) was added 2,2-dimethyloxirane (210 g, 2.92 mol) at room temperature. The reaction mixture was stirred at 100° C. for overnight. The reaction mixture was filtered. The filtrates were diluted with EtOAc (1000 mL) and washed with H₂O (1000 mL). The aqueous was extracted with EtOAc (1000 mL×2). The combined organics were concentrated. The residue was triturated by H₂O (500 mL) to give the crude product (120 g), which was recrystallized by n-Hexane to give the titled compound (71 g).

### Step B: 3,3-dimethyl-2,3-dihydrobenzo[b][1,4]dioxine-6-carbaldehyde

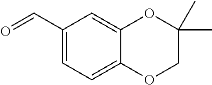

To a solution of 3,3-dimethyl-2,3-dihydrobenzo[b][1,4]dioxine-6-carbonitrile (71 g, 0.37 mol) in THE (700 mL) was added DIBAL-H (550 mL, 0.55 mol, 1M in n-Hexane) slowly at 0° C. The reaction mixture was stirred at 0° C. for 2 hours. The reaction was monitored by HPLC (quenched by 3N HCl). After completely disappearance of 3,3-dimethyl-2,3-dihydrobenzo[b][1,4]dioxine-6-carbonitrile, the reaction was quenched by 2N HCl slowly at 0° C. The resulting mixture was filtered. The filtrates were concentrated. The residue was dissolved in EtOAc (1000 mL) and washed with 1N HCl (1000 mL×2). The organic phases were concentrated to dryness. The crude product was purification by flash column chromatography to give the titled compound (64 g).

### Step C: 1-(3,3-dimethyl-2,3-dihydrobenzo[b][1,4]dioxin-6-yl)ethan-1-ol

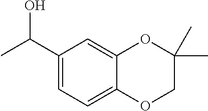

To a solution of 3,3-dimethyl-2,3-dihydrobenzo[b][1,4]dioxine-6-carbaldehyde (31 g, 0.16 mol) in THE (300 mL) was added MeMgBr (106 mL, 0.32 mmol, 3M in Et₂O) slowly at 0° C. The resulting mixture was stirred at r.t for 2 hours. The reaction mixture was quenched with H₂O slowly at 0° C., then filtered. The filtrates were extracted with EtOAc (500 mL×2). The combined organic phases were concentrated to give the crude product (30 g, contained 1.9% BP 1). The crude product was dissolved in DCM (300 mL), TEA (29.1 g, 2 eq), TrtCl (16 g, 0.4 eq) were added successively. The reaction was stirred at r.t for 2 hours. The reaction was monitored by LCMS. After completely disappearance of BP 1, the reaction was diluted with DCM (300 mL) and washed with H₂O (300 mL). The organic phases were concentrated to dryness. The resulting residue was purified by flash column chromatography to give the titled compound (26 g).

### Step D: 2-(6-((2R,5R)-4-(1-(3,3-dimethyl-2,3-dihydrobenzo[b][1,4]dioxin-6-yl)ethyl)-2-(methoxymethyl)-5-methylpiperazin-1-yl)-3,9-dimethyl-2-oxo-3,9-dihydro-2H-purin-8-yl)acetonitrile

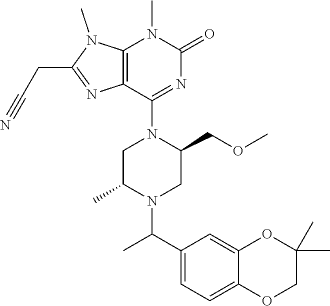 [SMILES: COC[C@H]1CN(C(C)c2ccc3c(c2)OC(C)(C)CO3)[C@H](C)CN1c1nc(=O)n(C)c2c1nc(CC#N)n2C]

A solution of 2-(6-((2R,5R)-2-(methoxymethyl)-5-methylpiperazin-1-yl)-3,9-dimethyl-2-oxo-3,9-dihydro-2H-purin-8-yl)acetonitrile (250 mg, 0.725 mol), 1-(3,3-dimethyl-2,3-dihydrobenzo[b][1,4]dioxin-6-yl)ethan-1-ol (302 mg, 1.449 mol), (cyanomethyl)trimethylphosphonium iodide (352 mg. 1.449 mol) and DIPEA (281 mg, 2.175 mol) in CH₃CN (3 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) to give the titled Compound A12 (240 mg), which was further separated into Compound A12a (70 mg) and Compound A12b (68 mg) by chiral Prep-HPLC. The chiral separation conditions are shown below.

| | |
|---|---|
| Column | CHIRALPAK ID |
| Column Size | 2 cm × 25 cm, 5 um |
| Mobile Phase A | Hex:DCM(0.5% 2M NH3—MeOH) |
| Mobile Phase B | MeOH |
| Flow Rate | 20 mL/min |
| Wave Length | UV 220 nm |
| Temperature | 25° C. |
| Prep-HPLC Equipment | Prep-HPLC-Gilson |

Compound A12a (the earlier peak): ¹H NMR (400 MHz, CD₃OD) δ 6.84-6.73 (m, 3H), 6.24 (s, 0.5H), 5.67-5.63 (m, 0.5H), 5.35-5.31 (m, 0.5H), 4.63-4.59 (m, 0.5H), 3.93 (s, 3H), 3.88 (s, 3H), 3.80-3.75 (m, 4H), 3.50-3.43 (m, 1H), 3.39 (s, 3H), 3.30 (s, 2H), 3.15-3.07 (m, 2H), 2.90 (s, 1H), 2.83-2.75 (m, 1H), 1.32 (s, 6H), 1.25 (d, J=6.4 Hz, 3H), 0.85 (d, J=6.5 Hz, 3H) ppm. MS: M/e 536 (M+1)⁺

Compound A12b (the later peak): ¹H NMR (400 MHz, CD₃OD) δ 6.81-6.72 (m, 3H), 6.03 (s, 0.5H), 5.89 (d, J=13.2 Hz, 0.5H), 5.13 (s, 0.5H), 4.84-4.79 (m, 0.5H), 3.93 (s, 3H), 3.87 (s, 2H), 3.78 (s, 3H), 3.75-3.68 (m, 1H), 3.66-3.56 (m, 2H), 3.54-3.49 (m, 1H), 3.40-3.30 (m, 4H), 3.29-3.18 (m, 3H), 2.60-2.40 (m, 1H), 1.31 (s, 6H), 1.27 (d, J=6.4 Hz, 3H), 0.99 (d, J=6.2 Hz, 3H) ppm. MS: M/e 536 (M+1)⁺

### Compound A13: 2-(6-((2R,5R)-2-(methoxymethyl)-5-methyl-4-(1-(3-methylquinoxalin-6-yl)ethyl)piperazin-1-yl)-3,9-dimethyl-2-oxo-3,9-dihydro-2H-purin-8-yl)acetonitrile

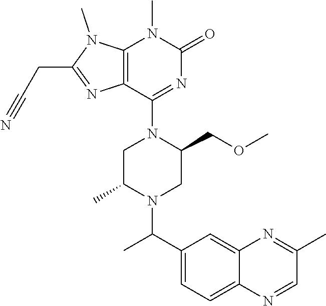 [SMILES: COC[C@H]1CN(C(C)c2ccc3ncc(C)nc3c2)[C@H](C)CN1c1nc(=O)n(C)c2c1nc(CC#N)n2C]

### Step A: 7-bromo-2-methylquinoxaline

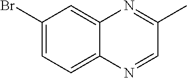

7-bromo-2-chloroquinoxaline (243 g, 1 mol) and Ferric acetylacetonate (17.63 g, 50 mmol) were dissolved in dry THE (1.5 L). A methyl magnesium bromide (3M in ethyl ether) solution (370 ml, 1.1 mol) was added dropwise at 0° C. After stirred for 1 hour, the reaction mixture was quenched with 1M aqueous HCl solution (1 L). The organic was removed under reduced pressure. DCM (1 L) was added to dilute the solid and filtered to separate the undissolved substance. The organic layer was washed with brine, dried over MgSO₄, filtered and the solvent was removed under vacuum. The residue (230 g) was slurried from ethanol (920 ml) for 2 hours and filtered. The solid (150 g) was dissolved in EtOAc (2 L) and decolorized with active carbon (70 g). The mixture was filtered and concentrated to give the titled compound (120 g, 54%). ¹H NMR (400 MHz, DMSO-d₆) δ 8.89 (s, 1H), 8.22 (d, J=2.0 Hz, 1H), 8.00 (d, J=8.9 Hz, 1H), 7.91 (dd, J=8.9, 2.0 Hz, 1H), 2.72 (s, 3H) ppm. MS: M/e 223 (M+1)⁺.

### Step B: 1-(3-methylquinoxalin-6-yl)ethan-1-one

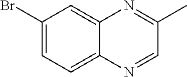

To a solution of 7-bromo-2-methylquinoxaline (66 g, 0.3 mol) and Cs₂CO₃ (147 g, 0.45 mol) in DMF/H₂O (8/1, 1.3 L) was added 1-(vinyloxy)butane (240 g, 2.4 mol), Pd(OAc)₂ (5.4 g, 0.024 mol) and DPPP (29.6 g, 0.072 mol). The mixture was stirred at 116° C. under N₂ for 16 h. The reaction was cooled to 0˜10° C., treated with 2N HCl (600 mL) drop wise, stirred at 0˜10° C. for one hour. The mixture was diluted with water (500 mL), a suspension was formed. The mixture was filtered through a pad of Celtic, washed with water (500 mL). The filtrate was extracted with EtOAc (2 L), washed with brine, dried over Na₂SO₄, filtered, concentrated. The water layer and brine layer were combined, extracted with DCM (1 L×2), washed with brine, dried over Na₂SO₄, filtered, concentrated to dryness. The combined residue was purified by flash column chromatography to give titled compound (35 g, 62%). ¹HNMR (400 MHz, CDCl₃) δ 8.83 (s, 1H), 8.60 (d, J=1.6 Hz, 1H), 8.28 (dd, J=1.6 Hz, 8.8 Hz, 1H), 8.13 (d, J=9.2 Hz, 1H), 2.82 (s, 3H), 2.76 (s, 3H) ppm. MS: M/e 187 (M+1)⁺.

### Step C: 1-(3-methylquinoxalin-6-yl)ethan-1-ol

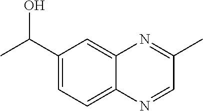

To a solution of 1-(3-methylquinoxalin-6-yl)ethan-1-one (55.4 g, 298 mmol) in EtOH (500 ml) at 0° C. was added NaBH₄ (6.8 g, 179 mmol) in some portions. After addition, the reaction was stirred at 0° C. for 1 hour. The reaction was quenched by adding water, most of EtOH was concentrated off under reduced pressure. The residue was diluted with water, extracted with DCM (500 mL×3), washed with brine, dried over Na₂SO₄, filtered, concentrated. The resulting residue was purified by flash column chromatography to give the titled compound (47.6 g, 85%). ¹H NMR (400 MHz, CDCl₃) δ 8.71 (s, 1H), 8.04 (d, J=8.8 Hz, 1H), 7.98 (d, J=0.8 Hz, 1H), 7.75 (dd, J=1.6 Hz, 8.4 Hz, 1H), 5.17-5.09 (m, 1H), 2.77 (s, 3H), 1.60 (d, J=6.8 Hz, 3H) ppm. MS: M/e 189 (M+1)⁺.

### Step D: 2-(6-((2R,5R)-2-(methoxymethyl)-5-methyl-4-(1-(3-methylquinoxalin-6-yl)ethyl)piperazin-1-yl)-3,9-dimethyl-2-oxo-3,9-dihydro-2H-purin-8-yl)acetonitrile

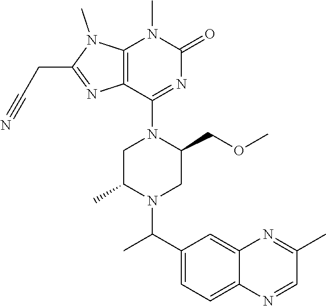 [SMILES: COC[C@H]1CN(C(C)c2ccc3ncc(C)nc3c2)[C@H](C)CN1c1nc(=O)n(C)c2c1nc(CC#N)n2C]

A solution of 2-(6-((2R,5R)-2-(methoxymethyl)-5-methylpiperazin-1-yl)-3,9-dimethyl-2-oxo-3,9-dihydro-2H-purin-8-yl)acetonitrile (43 mg, 0.125 mol), 1-(3-methylquinoxalin-6-yl)ethan-1-ol (47 mg, 0.249 mol), (cyanomethyl)trimethylphosphonium iodide (61 mg, 0.249 mol) and DIPEA (48 mg, 0.375 mol) in CH₃CN (1 ml). The mixture solution was degassed 3 times under N₂ atmosphere. Then the mixture solution was stirred at 105° C. for 24 hours. The reaction mixture was concentrated under reduced pressure to give the titled Compound A13 (crude), which was further separated into Compound A13a (6 mg) and Compound A13b (4 mg) by Prep-HPLC(Method A).

Compound A13a (the earlier peak): ¹H NMR (400 MHz, CD₃OD) δ 8.78 (s, 1H), 8.03 (d, J=8.6 Hz, 1H), 7.94 (s, 1H), 7.89 (dd, J=8.7, 1.5 Hz, 1H), 6.16-5.85 (m, 1H), 5.13 (s, 0.5H), 4.96-4.91 (m, 0.5H), 4.19 (d, J=25.6 Hz, 1H), 3.94 (s, 3H), 3.79 (s, 3H), 3.76 (d, J=6.6 Hz, 1H), 3.72-3.63 (m, 3.5H), 3.45-3.39 (m, 0.5H), 3.19 (s, 3H), 2.83-2.77 (m, 0.5H), 2.76 (s, 3H), 2.66-2.59 (m, 1.5H), 2.45 (d, J=12.1 Hz, 1H), 1.43 (d, J=6.5 Hz, 3H), 1.09 (d, J=6.5 Hz, 3H) ppm. MS: M/e 516 (M+1)⁺.

Compound A13b (the later peak): ¹H NMR (400 MHz, CD₃OD) δ 8.79 (s, 1H), 8.05 (d, J=8.6 Hz, 1H), 7.96 (s, 1H), 7.90 (dd, J=8.7, 1.6 Hz, 1H), 6.31 (s, 0.5H), 5.68-5.62 (m, 0.5H), 5.40 (s, 0.5H), 4.63-4.59 (m, 0.5H), 4.19 (d, J=33.4 Hz, 1H), 4.08-3.86 (m, 5H), 3.89-3.78 (m, 1H), 3.82 (s, 3H), 3.56-3.48 (m, 0.5H), 3.41 (s, 3H), 3.23-3.14 (m, 1.5H), 2.95-2.82 (m, 2.5H), 2.77-2.63 (m, 3.5H), 1.42 (d, J=6.5 Hz, 3H), 0.94 (d, J=6.5 Hz, 3H) ppm. MS: M/e 516 (M+1)⁺.

### Compound A14: 2-(6-fluoro-7-((2S,5R)-4-(1-(4-fluoro-2-(trifluoromethyl)phenyl)ethyl)-2,5-dimethylpiperazin-1-yl)-3,4-dimethyl-5-oxo-4,5-dihydro-3H-imidazo[4,5-b]pyridin-2-yl)acetonitrile

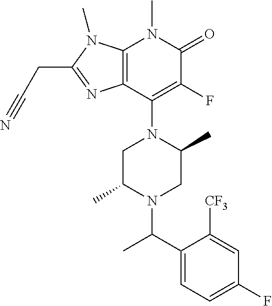 [SMILES: CC(c1ccc(F)cc1C(F)(F)F)N1C[C@H](C)N(c2c(F)c(=O)n(C)c3c2nc(CC#N)n3C)C[C@H]1C]

### Step A: tert-butyl(2R,5S)-4-(6-fluoro-2-formyl-3,4-dimethyl-5-oxo-4,5-dihydro-3H-imidazo[4,5-b]pyridin-7-yl)-2,5-dimethylpiperazine-1-carboxylate

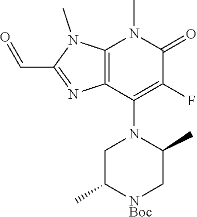 [SMILES: C[C@@H]1CN(c2c(F)c(=O)n(C)c3c2nc(C=O)n3C)[C@@H](C)CN1C(=O)OC(C)(C)C]

To a solution of tert-butyl (2R,5S)-4-(2-formyl-3,4-dimethyl-5-oxo-4,5-dihydro-3H-imidazo[4,5-b]pyridin-7-yl)-2,5-dimethylpiperazine-1-carboxylate (5.5 g, 13.7 mmol) in THE (30 mL) were added 1-Chloromethyl-4-fluoro-1,4-diazoniabicyclo[2.2.2]octane bis(tetrafluoroborate) (7.2 g, 20.5 mmol). The resulting mixture was stirred at 40° C. overnight. The reaction solvent was collected by filtration and concentrated under reduced pressure. The resulting residue was purified by flash column chromatography to give the titled compound (2.5 g, 43%). MS: M/e 422 (M+1)⁺.

### Step B: tert-butyl(2R,5S)-4-(6-fluoro-2-(hydroxymethyl)-3,4-dimethyl-5-oxo-4,5-dihydro-3H-imidazo[4,5-b]pyridin-7-yl)-2,5-dimethylpiperazine-1-carboxylate

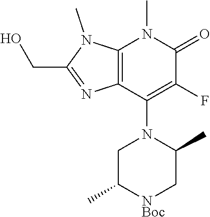 [SMILES: C[C@@H]1CN(c2c(F)c(=O)n(C)c3c2nc(CO)n3C)[C@@H](C)CN1C(=O)OC(C)(C)C]

To a solution of tert-butyl(2R,5S)-4-(6-fluoro-2-formyl-3,4-dimethyl-5-oxo-4,5-dihydro-3H-imidazo[4,5-b]pyridin-7-yl)-2,5-dimethylpiperazine-1-carboxylate (3 g, 7.1 mmol) in MeOH (10 mL) were added NaBH₄ (270 mg, 7.1 mmol). The resulting mixture was stirred at 0° C. for 5 min. The reaction solvent was diluted with DCM and washed with water, the organic layer was concentrated to dryness. The resulting residue was purified by flash column chromatography to give the titled compound (2.4 g, 73%). MS: M/e 424 (M+1)⁺.

### Step C: tert-butyl(2R,5S)-4-(2-(chloromethyl)-6-fluoro-3,4-dimethyl-5-oxo-4,5-dihydro-3H-imidazo[4,5-b]pyridin-7-yl)-2,5-dimethylpiperazine-1-carboxylate

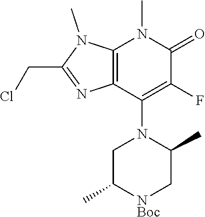 [SMILES: C[C@@H]1CN(c2c(F)c(=O)n(C)c3c2nc(CCl)n3C)[C@@H](C)CN1C(=O)OC(C)(C)C]

To a solution of SOCl₂ (1.4 g, 11.8 mmol) in DCM (600 mL) were added tert-butyl(2R,5S)-4-(6-fluoro-2-(hydroxymethyl)-3,4-dimethyl-5-oxo-4,5-dihydro-3H-imidazo[4,5-b]pyridin-7-yl)-2,5-dimethylpiperazine-1-carboxylate (1 g, 2.36 mmol). The resulting mixture was stirred at RT for 10 min. The reaction solvent was washed with water, aq. NaHCO₃ and brine. The organic layer was concentrated to give the titled compound which was used to next step directly without further purification.

### Step D: tert-butyl(2R,5S)-4-(2-(cyanomethyl)-6-fluoro-3,4-dimethyl-5-oxo-4,5-dihydro-3H-imidazo[4,5-b]pyridin-7-yl)-2,5-dimethylpiperazine-1-carboxylate

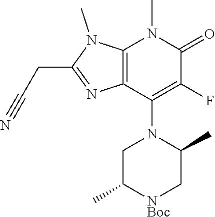 [SMILES: C[C@@H]1CN(c2c(F)c(=O)n(C)c3c2nc(CC#N)n3C)[C@@H](C)CN1C(=O)OC(C)(C)C]

To the solution of tert-butyl(2R,5S)-4-(2-(chloromethyl)-6-fluoro-3,4-dimethyl-5-oxo-4,5-dihydro-3H-imidazo[4,5-b]pyridin-7-yl)-2,5-dimethylpiperazine-1-carboxylate (crude) in CH₃CN (30 mL) were added TMSCN (1.17 g, 11.8 mmol) Cs₂CO₃ (3.8 g, 11.8 mmol). The resulting mixture was stirred at 60° C. for 30 min. The reaction solvent was removed under vacuum. The crude product was purified by flash column chromatography to give the titled compound (400 mg). MS: M/e 433 (M+1)⁺.

### Step E: 2-(7-((2S,5R)-2,5-dimethylpiperazin-1-yl)-6-fluoro-3,4-dimethyl-5-oxo-4,5-dihydro-3H-imidazo[4,5-b]pyridin-2-yl)acetonitrile

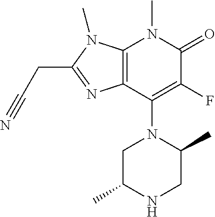 [SMILES: C[C@@H]1CN(c2c(F)c(=O)n(C)c3c2nc(CC#N)n3C)[C@@H](C)CN1]

To a solution of tert-butyl(2R,5S)-4-(2-(cyanomethyl)-6-fluoro-3,4-dimethyl-5-oxo-4,5-dihydro-3H-imidazo[4,5-b]pyridin-7-yl)-2,5-dimethylpiperazine-1-carboxylate (400 mg, 0.93 mmol) in DCM (10 mL) were added TFA (3 mL). The resulting mixture was stirred at rt for 2 hours. The reaction solvent was removed under vacuum. The crude product was dissolved with water and adjusted to pH=9 with aq. NaHCO₃. The aqueous layer was extracted with (DCM/IPA=3/1) to give the titled compound (400 mg, crude). MS: M/e 333 (M+1)⁺.

### Step F: 2-(6-fluoro-7-((2S,5R)-4-(1-(4-fluoro-2-(trifluoromethyl)phenyl)ethyl)-2,5-dimethylpiperazin-1-yl)-3,4-dimethyl-5-oxo-4,5-dihydro-3H-imidazo[4,5-b]pyridin-2-yl)acetonitrile

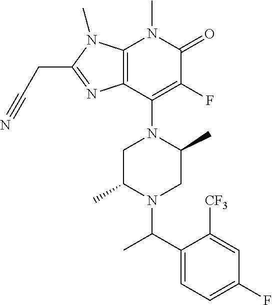 [SMILES: CC(c1ccc(F)cc1C(F)(F)F)N1C[C@H](C)N(c2c(F)c(=O)n(C)c3c2nc(CC#N)n3C)C[C@H]1C]

To a solution of 2-(7-((2S,5R)-2,5-dimethylpiperazin-1-yl)-6-fluoro-3,4-dimethyl-5-oxo-4,5-dihydro-3H-imidazo[4,5-b]pyridin-2-yl)acetonitrile (30 mg, 0.09 mmol) in CH₃CN (3 mL) and was added 1-(4-fluoro-2-(trifluoromethyl)phenyl)ethan-1-ol (57 mg, 0.27 mmol), (cyanomethyl)trimethylphosphonium iodide (110 mg, 0.45 mmol) and DIPEA (117 mg, 0.9 mmol). The resulting mixture was stirred at 105° C. overnight. The reaction solvent was removed under reduced pressure. The resulting residue was purified by Prep-TLC (DCM:MeOH=20:1) to give the titled Compound 14, which was further separated into Compound A14a (2 mg) and Compound A14b (1 mg) by Prep-HPLC(Method A).

Compound A14a (the earlier peak): ¹H NMR (400 MHz, CD₃OD) δ 8.10-8.02 (m, 1H), 7.39 (t, J=8.2 Hz, 2H), 4.66 (br s, 1H), 4.23-4.09 (m, 2H), 4.02-3.93 (m, 5H), 3.89 (s, 3H), 3.56 (s, 1H), 2.95-2.65 (m, 2H), 1.95 (d, J=12.2 Hz, 1H), 1.31 (d, J=6.4 Hz, 3H), 1.26 (d, J=6.7 Hz, 3H), 1.15 (d, J=6.5 Hz, 3H) ppm. MS: M/e 523 (M+1)⁺.

Compound A14b (the later peak): ¹H NMR (400 MHz, CD₃OD) δ 8.17-8.06 (m, 1H), 7.47-7.35 (m, 2H), 4.95 (br s, 1H), 4.24-3.95 (m, 2H), 3.98 (s, 3H), 3.89 (s, 3H), 3.80 (q, J=13.1 Hz, 2H), 3.17-3.07 (m, 1H), 2.89 (d, J=10.9 Hz, 1H), 2.75-2.66 (m, 2H), 1.52 (d, J=6.6 Hz, 3H), 1.25 (d, J=6.4 Hz, 3H), 1.00 (d, J=6.5 Hz, 3H) ppm. MS: M/e 523 (M+1)⁺.

### Compound A16: 2-(6-fluoro-7-((2S,5R)-4-(1-(4-fluoro-2-methoxyphenyl)ethyl)-2,5-dimethylpiperazin-1-yl)-3,4-dimethyl-5-oxo-4,5-dihydro-3H-imidazo[4,5-b]pyridin-2-yl)acetonitrile

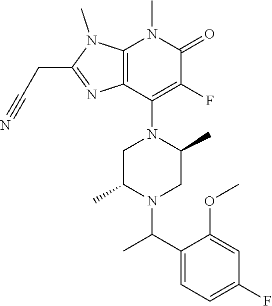 [SMILES: COc1cc(F)ccc1C(C)N1C[C@H](C)N(c2c(F)c(=O)n(C)c3c2nc(CC#N)n3C)C[C@H]1C]

To a solution of 2-(7-((2S,5R)-2,5-dimethylpiperazin-1-yl)-6-fluoro-3,4-dimethyl-5-oxo-4,5-dihydro-3H-imidazo[4,5-b]pyridin-2-yl)acetonitrile (30 mg, 0.09 mmol) in CH₃CN (3 mL) and was added 1-(4-fluoro-2-methoxyphenyl)ethan-1-ol (46 mg, 0.27 mmol), (cyanomethyl)trimethylphosphonium iodide (110 mg, 0.45 mmol) and DIPEA (117 mg, 0.9 mmol). The resulting mixture was stirred at 105° C. overnight. The reaction solvent was removed under reduced pressure. The resulting residue was purified by Prep-HPLC (Method A) to give the titled compound (4 mg, 8%). ¹H NMR (400 MHz, DMSO-d₆) 7.56-7.42 (m, 1H), 6.95-6.67 (m, 2H), 4.68-4.42 (m, 1H), 4.37 (d, J=3.8 Hz, 2H), 4.11-3.90 (m, 1H), 3.80 (s, 3H), 3.80 (d, J=2.9 Hz, 3H), 3.76 (s, 3H), 3.68-3.51 (m, 1H), 3.36-3.34 (m, 1H), 2.96-2.55 (m, 2H), 2.07 (d, J=11.4 Hz, 1H), 1.30 (d, J=6.4 Hz, 2H), 1.24-1.12 (m, 4H), 1.04 (d, J=6.4 Hz, 1H), 0.91 (d, J=6.4 Hz, 2H) ppm. MS: M/e 485 (M+1)⁺.

### Compound A18: 2-(7-((2S,5R)-4-(1-(2,3-dihydrobenzo[b][1,4]dioxin-6-yl)ethyl)-2,5-dimethylpiperazin-1-yl)-6-fluoro-3,4-dimethyl-5-oxo-4,5-dihydro-3H-imidazo[4,5-b]pyridin-2-yl)acetonitrile

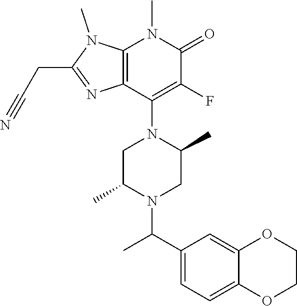 [SMILES: CC(c1ccc2c(c1)OCCO2)N1C[C@H](C)N(c2c(F)c(=O)n(C)c3c2nc(CC#N)n3C)C[C@H]1C]

To a solution of 2-(7-((2S,5R)-2,5-dimethylpiperazin-1-yl)-6-fluoro-3,4-dimethyl-5-oxo-4,5-dihydro-3H-imidazo[4,5-b]pyridin-2-yl)acetonitrile (30 mg, 0.09 mmol) in CH₃CN (3 mL) and was added 1-(2,3-dihydrobenzo[b][1,4]dioxin-6-yl)ethan-1-ol (49 mg, 0.27 mmol), (cyanomethyl)trimethylphosphonium iodide (110 mg, 0.45 mmol) and DIPEA (117 mg, 0.9 mmol). The resulting mixture was stirred at 105° C. overnight. The reaction solvent was removed under reduced pressure to give the titled Compound A18, which was further separated into Compound A18a (1 mg) and Compound A18b (2 mg) by Prep-HPLC (Method A).

Compound A18a (the earlier peak): ¹H NMR (400 MHz, DMSO-d₆) δ 6.82 (d, J=7.1 Hz, 1H), 6.81 (s, 2H), 4.56 (s, 1H), 4.38 (s, 2H), 4.23 (s, 4H), 3.88 (s, 3H), 3.76 (s, 3H), 3.64-3.42 (m, 3H), 2.91 (d, J=8.6 Hz, 1H), 2.68 (s, 1H), 2.58-2.56 (m, 1H), 1.26 (d, J=6.4 Hz, 3H), 1.21 (d, J=6.5 Hz, 3H), 0.92 (d, J=6.4 Hz, 3H) ppm. MS: M/e 495 (M+1)⁺.

Compound A18b (the later peak): ¹H NMR (400 MHz, DMSO-d₆) δ 6.84 (s, 1H), 6.79 (t, J=8.3 Hz, 2H), 4.40 (s, 1H), 4.37 (s, 2H), 4.21 (s, 4H), 3.87 (s, 3H), 3.76 (s, 5H), 3.44 (d, J=6.6 Hz, 1H), 3.33-3.31 (m, 1H), 2.64 (d, J=8.0 Hz, 1H), 2.10 (d, J=9.1 Hz, 1H), 1.20 (d, J=6.3 Hz, 3H), 1.13 (d, J=6.4 Hz, 3H), 1.03 (d, J=6.4 Hz, 3H) ppm. MS: M/e 495 (M+1)⁺.

### Compound A19: 2-(7-((2S,5R)-4-(1-(3,3-dimethyl-2,3-dihydrobenzo[b][1,4]dioxin-6-yl)ethyl)-2,5-dimethylpiperazin-1-yl)-6-fluoro-3,4-dimethyl-5-oxo-4,5-dihydro-3H-imidazo[4,5-b]pyridin-2-yl)acetonitrile

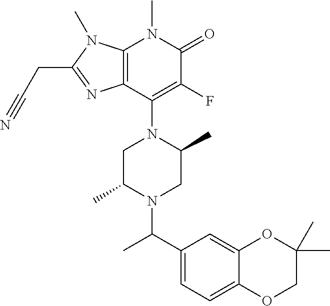 [SMILES: CC(c1ccc2c(c1)OC(C)(C)CO2)N1C[C@H](C)N(c2c(F)c(=O)n(C)c3c2nc(CC#N)n3C)C[C@H]1C]

A solution of 2-(7-((2S,5R)-2,5-dimethylpiperazin-1-yl)-6-fluoro-3,4-dimethyl-5-oxo-4,5-dihydro-3H-imidazo[4,5-b]pyridin-2-yl)acetonitrile (250 mg, 0.753 mol), 1-(3,3-dimethyl-2,3-dihydrobenzo[b][1,4]dioxin-6-yl)ethan-1-ol (313 mg, 1.506 mol), (cyanomethyl)trimethylphosphonium iodide (366 mg. 1.506 mol) and DIPEA (292 mg, 2.259 mol) in CH₃CN (3 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) to give the titled Compound A19 (60 mg), which was further separated into Compound A19a (18 mg) and Compound A19b (18 mg) by chiral Prep-HPLC. The chiral separation conditions are shown below.

| | |
|---|---|
| Column | CHIRALPAK IH |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A19a (the earlier peak): ¹H NMR (400 MHz, CD₃OD) δ 6.83-6.77 (m, 3H), 4.67 (s, 1H), 4.58 (s, 1H), 3.98 (s, 3H), 3.93 (s, 3H), 3.89 (s, 2H), 3.72-3.61 (m, 3H), 3.04 (dd, J=11.5, 3.3 Hz, 1H), 2.85-2.75 (m, 1H), 2.62 (dd, J=11.5, 3.7 Hz, 1H), 1.36 (d, J=6.4 Hz, 3H), 1.33 (s, 6H), 1.30 (d, J=6.5 Hz, 4H), 1.01 (d, J=6.4 Hz, 3H) ppm. MS: M/e 523 (M+1)⁺.

Compound A19b (the later peak): ¹H NMR (400 MHz, CD₃OD) δ 6.82 (d, J=6.6 Hz, 2H), 6.77 (d, J=8.8 Hz, 1H), 4.60-4.53 (m, 2H), 4.06-3.95 (m, 1H), 3.98 (s, 3H), 3.93-3.89 (m, 1H), 3.90 (s, 3H), 3.87 (s, 2H), 3.5-3.39 (m, 2H), 2.76 (dd, J=11.7, 3.2 Hz, 1H), 2.21 (d, J=11.3 Hz, 1H), 1.31 (d, J=2.7 Hz, 6H), 1.28 (d, J=6.5 Hz, 4H), 1.24 (d, J=6.5 Hz, 3H), 1.11 (d, J=6.4 Hz, 3H) ppm. MS: M/e 523 (M+1)⁺.

### Compound A20: 2-(7-((2S,5R)-4-(1-(3,3-dimethyl-2,3-dihydrobenzo[b][1,4]dioxin-6-yl)ethyl)-2,5-dimethylpiperazin-1-yl)-3-ethyl-6-fluoro-4-methyl-5-oxo-4,5-dihydro-3H-imidazo[4,5-b]pyridin-2-yl)acetonitrile

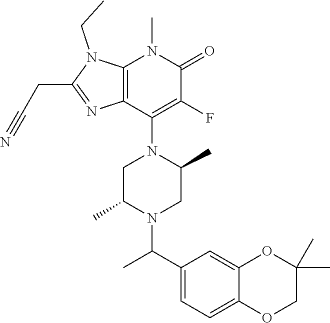 [SMILES: CCn1c(CC#N)nc2c(N3C[C@@H](C)N(C(C)c4ccc5c(c4)OC(C)(C)CO5)C[C@@H]3C)c(F)c(=O)n(C)c21]

### Step A: tert-butyl (2R,5S)-4-(5-chloro-3-ethyl-3H-imidazo[4,5-b]pyridin-7-yl)-2,5-dimethylpiperazine-1-carboxylate

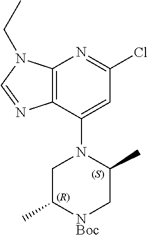

To a solution of tert-butyl (2R,5S)-4-(5-chloro-3H-imidazo[4,5-b]pyridin-7-yl)-2,5-dimethylpiperazine-1-carboxylate (49 g, 134.2 mmol) and K₂CO₃ (55.6 g, 403 mmol) in acetone (300 mL) was added CH₃CH₂I (41.9 g, 268.4 mmol). The reaction was stirred at room temperature for 16 hours. After filtration, the reaction mixture was diluted with water, extracted with EtOAc (300 mL×2), washed with brine, dried over Na₂SO₄, filtered, and concentrated to dryness. The resulting residue was purified by flash column chromatography (PE:EtOAc=1:1) to give the titled compound (23.5 g, 45%). MS: M/e 394 (M+1)⁺.

### Step B: tert-butyl (2R,5S)-4-(3-ethyl-5-hydroxy-3H-imidazo[4,5-b]pyridin-7-yl)-2,5-dimethylpiperazine-1-carboxylate

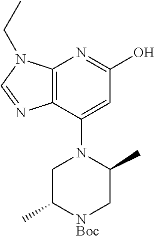 [SMILES: CCn1cnc2c(N3C[C@@H](C)N(C(=O)OC(C)(C)C)C[C@@H]3C)cc(O)nc21]

To a solution of tert-butyl (2R,5S)-4-(5-chloro-3-ethyl-3H-imidazo[4,5-b]pyridin-7-yl)-2,5-dimethylpiperazine-1-carboxylate (23.5 g, 60 mmol), Pd₂(dba)₃ (5.47 g, 6 mmol), t-BuXphos (5.11 g, 12 mmol) and KOH (60 mL, 3M, 180 mmol) in dioxane (120 mL) was degassed 3 times under N₂ atmosphere. Then the mixture was stirred at 90° C. for 12 hours. The reaction mixture was diluted with water and extracted with EtOAc (150 mL×2). The combined organic layers were washed with brine, dried over Na₂SO₄, filtered and concentrated to dryness. The resulting residue was purified by flash column chromatography (DCM:MeOH=10:1) to give the titled compound (15.6 g, 69%). MS: M/e 376 (M+1)⁺.

### Step C: tert-butyl (2R,5S)-4-(3-ethyl-4-methyl-5-oxo-4,5-dihydro-3H-imidazo[4,5-b]pyridin-7-yl)-2,5-dimethylpiperazine-1-carboxylate

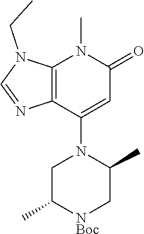 [SMILES: CCn1cnc2c(N3C[C@@H](C)N(C(=O)OC(C)(C)C)C[C@@H]3C)cc(=O)n(C)c21]

To a solution of tert-butyl (2R,5S)-4-(3-ethyl-5-hydroxy-3H-imidazo[4,5-b]pyridin-7-yl)-2,5-dimethylpiperazine-1-carboxylate (7.5 g, 20 mmol) and Cs₂CO₃ (22.8 g, 60 mmol) in dioxane (210 mL) was added CH₃I (28.4 g, 200 mmol). The reaction was stirred at room temperature for 16 hours. After filtration, the reaction mixture was diluted with water, extracted with EtOAc (150 mL×2), washed with brine, dried over Na₂SO₄, filtered, and concentrated to dryness. The resulting residue was purified by flash column chromatography (DCM:MeOH=20:1) to give the titled compound (5 g, 64%). MS: M/e 390 (M+1)⁺.

### Step D: tert-butyl (2R,5S)-4-(3-ethyl-2-formyl-4-methyl-5-oxo-4,5-dihydro-3H-imidazo[4,5-b]pyridin-7-yl)-2,5-dimethylpiperazine-1-carboxylate

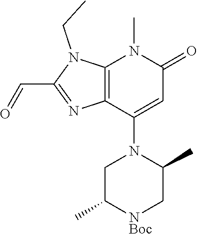 [SMILES: CCn1c(C=O)nc2c(N3C[C@@H](C)N(C(=O)OC(C)(C)C)C[C@@H]3C)cc(=O)n(C)c21]

To a solution of tert-butyl (2R,5S)-4-(3-ethyl-4-methyl-5-oxo-4,5-dihydro-3H-imidazo[4,5-b]pyridin-7-yl)-2,5-dimethylpiperazine-1-carboxylate (5.0 g, 12.853 mmol) in THE (150 mL) was degassed 3 times under N₂ atmosphere. Then added LDA (19.3 mL, 2M, 38.560 mmol) dropwise at −78° C. The mixture was stirred at −78° C. for 1 hours. Added DMF (9.39 g, 128.53 mmol) to quench the reaction solution. The reaction mixture was stirred for 1 hour and diluted with saturated NH₄Cl, extracted with EtOAc (150 mL×2), washed with brine, dried over Na₂SO₄, filtered, and concentrated to dryness. The resulting residue was purified by flash column chromatography (DCM:MeOH=20:1) to give the titled compound (5.0 g, 93%). MS: M/e 418 (M+1)⁺.

### Step E: tert-butyl (2R,5S)-4-(3-ethyl-6-fluoro-2-formyl-4-methyl-5-oxo-4,5-dihydro-3H-imidazo[4,5-b]pyridin-7-yl)-2,5-dimethylpiperazine-1-carboxylate

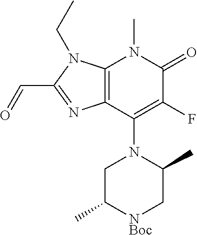 [SMILES: CCn1c(C=O)nc2c(N3C[C@@H](C)N(C(=O)OC(C)(C)C)C[C@@H]3C)c(F)c(=O)n(C)c21]

To a solution of tert-butyl (2R,5S)-4-(3-ethyl-2-formyl-4-methyl-5-oxo-4,5-dihydro-3H-imidazo[4,5-b]pyridin-7-yl)-2,5-dimethylpiperazine-1-carboxylate (1.0 g, 2.40 mmol) in THE (60 mL) was added Selectfluor (2.55 g, 7.20 mmol). The reaction was stirred at 60° C. for 6 hours. After filtration, the reaction mixture was concentrated to dryness. The resulting residue was purified by flash column chromatography (PE:EtOAc=1:1) to give the titled compound (450 mg, 43%). MS: M/e 436 (M+1)⁺.

### Step F: tert-butyl (2R,5S)-4-(3-ethyl-6-fluoro-2-(hydroxymethyl)-4-methyl-5-oxo-4,5-dihydro-3H-imidazo[4,5-b]pyridin-7-yl)-2,5-dimethylpiperazine-1-carboxylate

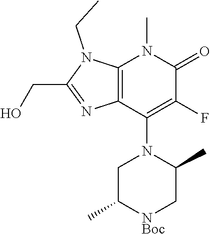 [SMILES: CCn1c(CO)nc2c(N3C[C@@H](C)N(C(=O)OC(C)(C)C)C[C@@H]3C)c(F)c(=O)n(C)c21]

To a solution of tert-butyl (2R,5S)-4-(3-ethyl-6-fluoro-2-formyl-4-methyl-5-oxo-4,5-dihydro-3H-imidazo[4,5-b]pyridin-7-yl)-2,5-dimethylpiperazine-1-carboxylate (450 mg, 1.034 mmol) in MeOH (10 mL) was added NaBH₄ (39 mg, 1.034 mmol). The reaction was stirred at room temperature for 1 hours. The reaction mixture was quenched with saturated NH₄Cl, extracted with EtOAc (30 mL×2), washed with brine, dried over Na₂SO₄, filtered, and concentrated to dryness. The resulting residue was purified by flash column chromatography (DCM:MeOH=10:1) to give the titled compound (350 mg, 77%). MS: M/e 438 (M+1)⁺.

### Step G: tert-butyl (2R,5S)-4-(2-(chloromethyl)-3-ethyl-6-fluoro-4-methyl-5-oxo-4,5-dihydro-3H-imidazo[4,5-b]pyridin-7-yl)-2,5-dimethylpiperazine-1-carboxylate

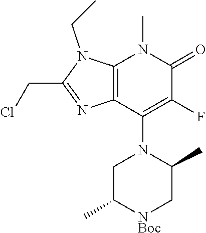 [SMILES: CCn1c(CCl)nc2c(N3C[C@@H](C)N(C(=O)OC(C)(C)C)C[C@@H]3C)c(F)c(=O)n(C)c21]

To a solution of tert-butyl (2R,5S)-4-(3-ethyl-6-fluoro-2-(hydroxymethyl)-4-methyl-5-oxo-4,5-dihydro-3H-imidazo[4,5-b]pyridin-7-yl)-2,5-dimethylpiperazine-1-carboxylate (350 mg, 0.801 mmol) in DCM (20 mL) was added SOCl₂ (100 mg, 0.841 mmol). The reaction was stirred at room temperature for 0.5 hours. The reaction mixture concentrated to give the titled compound (350 mg, 96%). MS: M/e 456 (M+1)⁺.

### Step H: tert-butyl (2R,5S)-4-(2-(cyanomethyl)-3-ethyl-6-fluoro-4-methyl-5-oxo-4,5-dihydro-3H-imidazo[4,5-b]pyridin-7-yl)-2,5-dimethylpiperazine-1-carboxylate

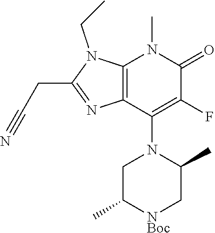 [SMILES: CCn1c(CC#N)nc2c(N3C[C@@H](C)N(C(=O)OC(C)(C)C)C[C@@H]3C)c(F)c(=O)n(C)c21]

To a solution of tert-butyl (2R,5S)-4-(2-(chloromethyl)-3-ethyl-6-fluoro-4-methyl-5-oxo-4,5-dihydro-3H-imidazo[4,5-b]pyridin-7-yl)-2,5-dimethylpiperazine-1-carboxylate (350 mg, 0.769 mmol) in DCM (20 mL) was added TBAF (1.54 mL, 1M, 1.538 mmol), followed TMSCN (153 mg, 1.538 mmol). The reaction was stirred at room temperature for 16 hours. The reaction mixture was diluted with water, extracted with DCM (40 mL×2), washed with brine, dried over Na₂SO₄, filtered, and concentrated to dryness. The resulting residue was purified by flash column chromatography (PE:EtOAc=1:5) to give the titled compound (300 mg, 87%). MS: M/e 447 (M+1)⁺.

### Step I: 2-(7-((2S,5R)-2,5-dimethylpiperazin-1-yl)-3-ethyl-6-fluoro-4-methyl-5-oxo-4,5-dihydro-3H-imidazo[4,5-b]pyridin-2-yl)acetonitrile

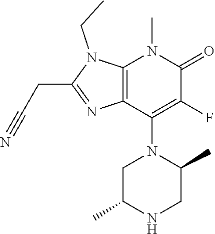 [SMILES: CCn1c(CC#N)nc2c(N3C[C@@H](C)NC[C@@H]3C)c(F)c(=O)n(C)c21]

To a stirred solution of tert-butyl (2R,5S)-4-(2-(cyanomethyl)-3-ethyl-6-fluoro-4-methyl-5-oxo-4,5-dihydro-3H-imidazo[4,5-b]pyridin-7-yl)-2,5-dimethylpiperazine-1-carboxylate (300 mg, 0.673 mmol) in CH₂Cl₂ (20 mL) was added TFA (5 mL). Then the reaction mixture was stirred at room temperature for 4 hours, concentrated to give a residue, basified to pH=10˜11 with saturated NaHCO₃ aq., and extracted with CH₂Cl₂/IPA (3/1, 50 mL×3). The combined organic layers were washed with brine, dried over Na₂SO₄, and concentrated to give the titled compound (180 mg, 78%). MS: M/e 347 (M+1)⁺.

### Step J: 2-(7-((2S,5R)-4-(1-(3,3-dimethyl-2,3-dihydrobenzo[b][1,4]dioxin-6-yl)ethyl)-2,5-dimethylpiperazin-1-yl)-3-ethyl-6-fluoro-4-methyl-5-oxo-4,5-dihydro-3H-imidazo[4,5-b]pyridin-2-yl)acetonitrile

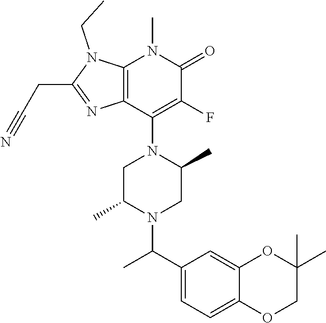 [SMILES: CCn1c(CC#N)nc2c(N3C[C@@H](C)N(C(C)c4ccc5c(c4)OC(C)(C)CO5)C[C@@H]3C)c(F)c(=O)n(C)c21]

A solution of 2-(7-((2S,5R)-2,5-dimethylpiperazin-1-yl)-3-ethyl-6-fluoro-4-methyl-5-oxo-4,5-dihydro-3H-imidazo[4,5-b]pyridin-2-yl)acetonitrile (180 mg, 0.520 mmol), 1-(3,3-dimethyl-2,3-dihydrobenzo[b][1,4]dioxin-6-yl)ethan-1-ol (216 mg, 1.040 mmol), (cyanomethyl) trimethyl phosphonium iodide (253 mg. 1.040 mmol) and DIPEA (201 mg, 1.560 mol) in CH₃CN (2 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) and Prep-HPLC(Method A) to give the titled compound (16 mg, 6%). ¹H NMR (400 MHz, CD₃OD) δ 6.88-6.84 (m, 3H), 4.59 (s, 1H), 4.42 (d, J=7.2 Hz, 2H), 4.26-4.21 (m, 0.5H), 4.08-3.99 (m, 0.5H), 3.89 (d, J=11.0 Hz, 6H), 3.76-3.49 (m, 2H), 3.13-3.04 (m, 2H), 3.03-2.31 (m, 2H), 1.44 (dd, J=17.7, 10.6 Hz, 6H), 1.33 (d, J=5.8 Hz, 8H), 1.21 (t, J=17.9 Hz, 4H) ppm. MS: M/e 537 (M+1)⁺

### Compound A21: 2-(7-((2S,5R)-4-(1-(3,3-dimethyl-2,3-dihydro-[1,4]dioxino[2,3-b]pyridin-6-yl)ethyl)-2,5-dimethylpiperazin-1-yl)-3-ethyl-6-fluoro-4-methyl-5-oxo-4,5-dihydro-3H-imidazo[4,5-b]pyridin-2-yl)acetonitrile

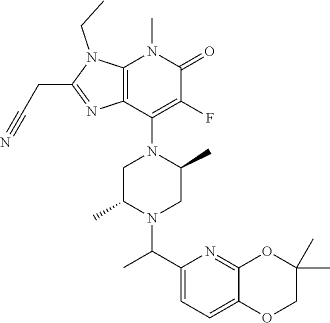 [SMILES: CCn1c(CC#N)nc2c(N3C[C@@H](C)N(C(C)c4ccc5c(n4)OC(C)(C)CO5)C[C@@H]3C)c(F)c(=O)n(C)c21]

### Step A: 2-chloro-6-(hydroxymethyl)pyridin-3-ol

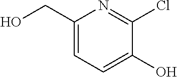

A solution of 2-chloropyridin-3-ol (2 g, 15.50 mmol), formaldehyde (37%, 12.6 g, 155 mmol), NaHCO₃ (3.9 g, 46.5 mmol) in water (50 mL) was stirred at 90° C. overnight. The reaction mixture was quenched with con. HCl and extracted with CH₂Cl₂/IPA (3/1, 10 mL×3). The combined organic layers were washed with brine, dried over Na₂SO₄, concentrated to give the titled compound (2.1 g, 85%) as colorless oil. MS: M/e 160 (M+1)⁺.

### Step B: 1-((2-chloro-6-(hydroxymethyl)pyridin-3-yl)oxy)propan-2-one

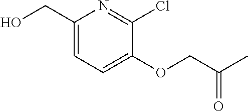

A solution of 2-chloro-6-(hydroxymethyl)pyridin-3-ol (2.1 g, 13.21 mmol), 1-chloropropan-2-one (1.46 g, 15.85 mmol), KI (219 mg, 1.32 mmol) and K₂CO₃ (3.65 g, 26.42 mmol) in ACN (100 mL) was stirred at 60° C. for 1 h. The mixture was diluted with CH₂Cl₂ (20 mL), then washed with H₂O, brine, dried over Na₂SO₄, concentrated to dryness. The resulting residue was purified by flash column chromatography to give the titled compound (2.7 g, 95%) as colorless oil. MS: M/e 216 (M+1)⁺.

### Step C: 6-chloro-5-(2-oxopropoxy)picolinaldehyde

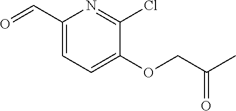

A mixture of 1-((2-chloro-6-(hydroxymethyl)pyridin-3-yl)oxy)propan-2-one (2.7 g, 12.56 mmol) and Dess-Martin Periodinane (6.4 g, 15.07 mmol) in DCM (30 mL) was stirred at r.t for 3 h. The reaction mixture was diluted with DCM and washed with water, concentrated to dryness. The resulting residue was purified by flash column chromatography to give the titled compound (2.6 g, 97%). MS: M/e 214 (M+1)⁺.

### Step D: 1-((2-chloro-6-(1-hydroxyethyl)pyridin-3-yl)oxy)-2-methylpropan-2-ol

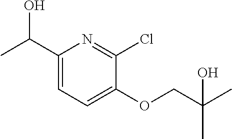 [SMILES: CC(O)c1ccc(OCC(C)(C)O)c(Cl)n1]

To a stirred solution of 6-chloro-5-(2-oxopropoxy)picolinaldehyde (2.6 g, 12.21 mmol) in dry THE (200 mL) was added dropwise MeMgBr (1.0 M, 30.5 mL, 30.5 mmol) at 0° C. After then, the mixture was stirred for half an hour. The reaction was quenched with aq.NH₄Cl, extracted with EtOAc (100 mL×3). The combined organic layers were washed with brine, dried over Na₂SO₄, concentrated to dryness. The resulting residue was purified by flash column chromatography to give the titled compound (2.6 g, 87%) as colorless oil. MS: M/e 246 (M+1)⁺.

### Step E: 1-(3,3-dimethyl-2,3-dihydro-[1,4]dioxino[2,3-b]pyridin-6-yl)ethan-1-ol

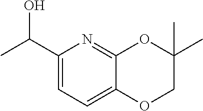

A mixture of 1-((2-chloro-6-(1-hydroxyethyl)pyridin-3-yl)oxy)-2-methylpropan-2-ol (1 g, 4.08 mmol), Pd(OAc)₂ (91 mg, 0.41 mmol) BINAP (254 mg, 0.41 mmol) and Cs₂CO₃ (2.66 g, 8.16 mmol) in toluene (30 mL) was stirred at 100° C. under N₂ overnight. The reaction mixture was extracted with EtOAc (100 mL×3). The combined organic layers were washed with brine, dried over Na₂SO₄, concentrated to dryness. The resulting residue was purified by flash column chromatography to give the titled compound (550 mg, 64%) as colorless oil. MS: M/e 210 (M+1)⁺.

### Step F: 2-(7-((2S,5R)-4-(1-(3,3-dimethyl-2,3-dihydro-[1,4]dioxino[2,3-b]pyridin-6-yl)ethyl)-2,5-dimethylpiperazin-1-yl)-3-ethyl-6-fluoro-4-methyl-5-oxo-4,5-dihydro-3H-imidazo[4,5-b]pyridin-2-yl)acetonitrile

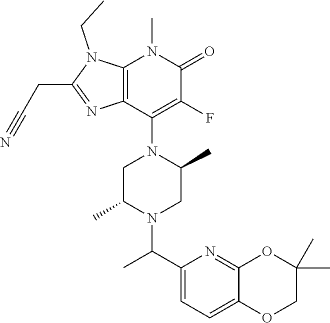 [SMILES: CCn1c(CC#N)nc2c(N3C[C@@H](C)N(C(C)c4ccc5c(n4)OC(C)(C)CO5)C[C@@H]3C)c(F)c(=O)n(C)c21]

A solution of 2-(7-((2S,5R)-2,5-dimethylpiperazin-1-yl)-3-ethyl-6-fluoro-4-methyl-5-oxo-4,5-dihydro-3H-imidazo[4,5-b]pyridin-2-yl)acetonitrile (120 mg, 0.347 mmol), 1-(3,3-dimethyl-2,3-dihydro-[1,4]dioxino[2,3-b]pyridin-6-yl)ethan-1-ol (218 mg, 1.040 mmol), (cyanomethyl)trimethylphosphonium iodide (337 mg. 1.388 mmol) and DIPEA (224 mg, 1.735 mol) in CH₃CN (2 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) and Prep-HPLC(Method A) to give the titled compound (11 mg, 6%). ¹H NMR (400 MHz, CD₃OD) δ 7.33 (t, J=6.1 Hz, 1H), 7.12 (t, J=8.0 Hz, 1H), 4.65 (d, J=34.1 Hz, 1H), 4.43 (dd, J=13.1, 6.0 Hz, 2H), 4.26-4.05 (m, 2H), 4.03-3.97 (m, 0.5H), 3.98 (s, 2H), 3.89 (s, 3H), 3.79-3.62 (m, 2H), 3.38-3.34 (m, 1.5H), 3.23-3.00 (m, 1H), 2.88-2.50 (m, 1H), 1.46 (t, J=7.3 Hz, 6H), 1.41 (s, 6H), 1.32 (d, J=6.4 Hz, 4H), 1.21 (d, J=6.3 Hz, 2H) ppm. MS: M/e 538 (M+1)⁺

### Compound A22: 2-(7-((2S,5R)-2,5-dimethyl-4-(1-(quinoxalin-6-yl)ethyl)piperazin-1-yl)-6-fluoro-3,4-dimethyl-5-oxo-4,5-dihydro-3H-imidazo[4,5-b]pyridin-2-yl)acetonitrile

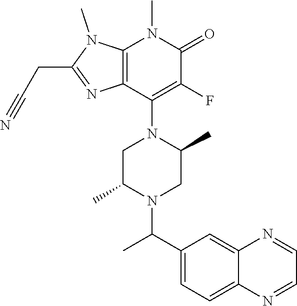 [SMILES: CC(c1ccc2nccnc2c1)N1C[C@H](C)N(c2c(F)c(=O)n(C)c3c2nc(CC#N)n3C)C[C@H]1C]

To a solution of 2-(7-((2S,5R)-2,5-dimethyl-4-(1-(quinoxalin-6-yl)ethyl)piperazin-1-yl)-3,4-dimethyl-5-oxo-4,5-dihydro-3H-imidazo[4,5-b]pyridin-2-yl)acetonitrile (20 mg, 0.0.04 mmol) in CH₃CN (0.2 mL) and THE (2 mL) and was added 1-Chloromethyl-4-fluoro-1,4-diazoniabicyclo[2.2.2]octane bis(tetrafluoroborate) (23 mg, 0.06 mmol). The resulting mixture was stirred for 10 min at rt. The reaction solvent was removed under reduced pressure. The resulting residue was purified by Prep-TLC to give the titled Compound A22, which was further separated into Compound A22a (0.63 mg) and Compound A22b (0.3 mg, crude) by Prep-HPLC(Method A).

Compound A22a (the earlier peak): ¹H NMR (400 MHz, DMSO-d₆) δ 8.93 (t, J=4.2 Hz, 2H), 8.18-7.90 (m, 3H), 4.37 (s, 2H), 4.01 (d, J=6.5 Hz, 1H), 3.93-3.83 (m, 4H), 3.76 (s, 3H), 3.69-3.61 (m, 1H), 3.51 (d, J=13.5 Hz, 1H), 3.06-2.93 (m, 2H), 2.68 (d, J=7.9 Hz, 1H), 1.42-1.29 (m, 5H), 1.20-1.08 (m, 2H), 0.97 (t, J=11.8 Hz, 2H) ppm. MS: M/e 489 (M+1)⁺.

### Compound A23: 2-(7-((2S,5R)-2,5-dimethyl-4-(1-(3-methylquinoxalin-6-yl)ethyl)piperazin-1-yl)-6-fluoro-3,4-dimethyl-5-oxo-4,5-dihydro-3H-imidazo[4,5-b]pyridin-2-yl)acetonitrile

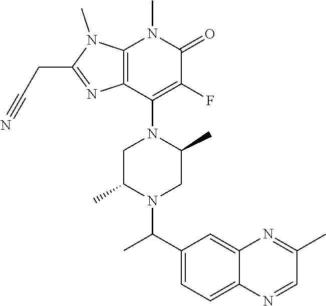 [SMILES: Cc1cnc2ccc(C(C)N3C[C@H](C)N(c4c(F)c(=O)n(C)c5c4nc(CC#N)n5C)C[C@H]3C)cc2n1]

A solution of 2-(7-((2S,5R)-2,5-dimethylpiperazin-1-yl)-6-fluoro-3,4-dimethyl-5-oxo-4,5-dihydro-3H-imidazo[4,5-b]pyridin-2-yl)acetonitrile (40 mg, 0.093 mol), 1-(3-methylquinoxalin-6-yl)ethan-1-ol (35 mg, 0.185 mol), (cyanomethyl)trimethylphosphonium iodide (45 mg. 0.185 mol) and DIPEA (36 mg, 0.279 mol) in CH₃CN (1 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) to give the titled Compound A23, which was further separated into Compound A23a (7 mg) and Compound A23b (4 mg) by Prep-HPLC(Method A).

Compound A23a (the earlier peak): ¹H NMR (400 MHz, CD₃OD) δ 8.79 (s, 1H), 8.05 (d, J=8.6 Hz, 1H), 7.98 (d, J=1.4 Hz, 1H), 7.93 (dd, J=8.7, 1.7 Hz, 1H), 4.84-4.76 (m, 2H), 4.23-4.18 (m, 1H), 4.07-3.98 (m, 1H), 3.96 (s, 3H), 3.89 (s, 3H), 3.85-3.75 (m, 2H), 3.17 (dd, J=11.5, 3.7 Hz, 1H), 2.84-2.78 (m, 2H), 2.77 (s, 3H), 1.48 (dd, J=6.5 Hz, 3H), 1.45 (dd, J=6.5 Hz, 3H), 1.07 (d, J=6.5 Hz, 3H) ppm. MS: M/e 503 (M+1)⁺.

Compound A23b (the later peak): ¹H NMR (400 MHz, CD₃OD) δ 8.78 (s, 1H), 8.02 (d, J=8.6 Hz, 1H), 7.98 (s, 1H), 7.95 (dd, J=8.6, 1.7 Hz, 1H), 4.84-4.80 (m, 1H), 4.63-4.54 (m, 1H), 4.25-1.28 (m, 1H), 4.10-3.99 (m, 2H), 3.98 (s, 3H), 3.90 (s, 3H), 3.88-3.83 (m, 1H), 3.61-3.50 (m, 1H), 2.90 (dd, J=11.9, 3.8 Hz, 1H), 2.76 (s, 3H), 2.16 (dd, J=11.8, 2.5 Hz, 1H), 1.44 (d, J=6.6 Hz, 3H), 1.27 (d, J=6.5 Hz, 3H), 1.20 (d, J=6.5 Hz, 3H) ppm. MS: M/e 503 (M+1)⁺.

### Compound A24: 2-(6-((2S,5R)-4-(1-(4-fluoro-2-methoxyphenyl)ethyl)-2,5-dimethylpiperazin-1-yl)-3,9-dimethyl-2-oxo-3,9-dihydro-2H-purin-8-yl)acetonitrile

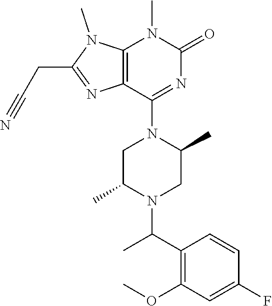 [SMILES: COc1cc(F)ccc1C(C)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (50 mg, 0.159 mol), 1-(4-fluoro-2-methoxyphenyl)ethan-1-ol (40 mg, 0.238 mol), (cyanomethyl)trimethylphosphonium iodide (77 mg. 0.318 mol) and DIPEA (62 mg, 0.477 mol) in CH₃CN (1 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) to give the titled Compound A24, which was further separated into Compound A24a (5 mg) and Compound A24b (3 mg) by Prep-HPLC (Method A).

Compound A24a (the earlier peak): ¹H NMR (400 MHz, CD₃OD) δ 7.56 (s, 1H), 6.90-6.64 (m, 2H), 5.99-5.94 (m, 0.5H), 5.08-5.04 (m, 0.5H), 4.85-4.41 (m, 1H), 4.29-4.03 (m, 2H), 3.93 (s, 3H), 3.83 (s, 3H), 3.79 (s, 3H), 3.70-3.58 (m, 2H), 2.95-2.64 (m, 2H), 2.56-2.20 (m, 1H), 1.43-1.35 (m, 3H), 1.30 (d, J=5.7 Hz, 3H), 1.18-1.01 (m, 3H) ppm. MS: M/e 468 (M+1)⁺.

Compound A24b (the later peak): ¹H NMR (400 MHz, CD₃OD) δ 7.52 (dd, J=8.4, 7.0 Hz, 1H), 6.80 (dd, J=11.2, 2.2 Hz, 1H), 6.74 (td, J=8.4, 2.3 Hz, 1H), 6.19-5.02 (m, 2H), 4.38-3.34 (m, 1H), 4.21 (s, 1H), 3.93 (s, 3H), 3.85 (s, 3H), 3.80 (s, 3H), 3.53-3.38 (m, 1.5H), 3.12-2.91 (m, 2H), 2.92-2.70 (m, 1.5H), 1.41 (d, J=6.3 Hz, 3H), 1.33 (d, J=6.5 Hz, 3H), 1.00 (d, J=6.1 Hz, 3H) ppm. MS: M/e 468 (M+1)⁺.

### Compound A25: 2-(6-((2S,5R)-4-(1-(2-(difluoromethoxy)-4-fluorophenyl)ethyl)-2,5-dimethylpiperazin-1-yl)-3,9-dimethyl-2-oxo-3,9-dihydro-2H-purin-8-yl)acetonitrile

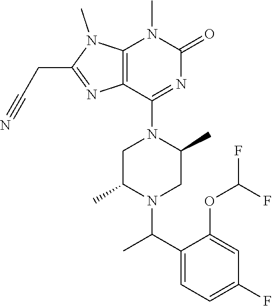 [SMILES: CC(c1ccc(F)cc1OC(F)F)N1C[C@H](C)N(c2nc(=O)n(C)c3c2nc(CC#N)n3C)C[C@H]1C]

A mixture of 2-(6-((2S,5R)-2,5-dimethylpiperazin-1-yl)-3,9-dimethyl-2-oxo-3,9-dihydro-2H-purin-8-yl)acetonitrile (50 mg, 0.158 mmol), 1-(2-(difluoromethoxy)-4-fluorophenyl)ethan-1-ol (65.4 mg, 0.317 mmol), (cyanomethyl)trimethylphosphonium iodide (115 mg, 0.474 mmol) and DIPEA (204 mg, 1.58 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to give the residue, which was purified by Pre-TLC (CH₂Cl₂/MeOH=10:1) to give the titled compound (18 mg, 23%). ¹H NMR (400 MHz, CD₃OD) δ 7.81-7.65 (m, 1H), 7.13-6.68 (m, 3H), 6.04-4.45 (m, 2H), 4.21 (s, 2H), 4.16-3.97 (m, 1H), 3.93 (s, 3H), 3.78 (s, 3H), 3.65-3.46 (m, 1H), 3.04-2.60 (m, 2.5H), 2.28-2.07 (m, 0.5H), 1.50-1.22 (m, 6H), 1.13-0.85 (m, 3H) ppm. MS: M/e 504 (M+1)⁺.

### Compound A26: 2-(6-((2S,5R)-4-(1-(4-fluoro-2-(trifluoromethoxy)phenyl)ethyl)-2,5-dimethylpiperazin-1-yl)-3,9-dimethyl-2-oxo-3,9-dihydro-2H-purin-8-yl)acetonitrile

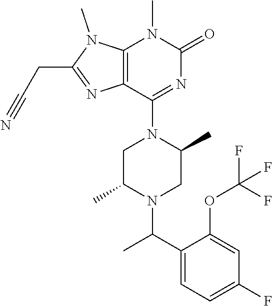 [SMILES: CC(c1ccc(F)cc1OC(F)(F)F)N1C[C@H](C)N(c2nc(=O)n(C)c3c2nc(CC#N)n3C)C[C@H]1C]

A mixture of 2-(6-((2S,5R)-2,5-dimethylpiperazin-1-yl)-3,9-dimethyl-2-oxo-3,9-dihydro-2H-purin-8-yl)acetonitrile (50 mg, 0.158 mmol), 1-(4-fluoro-2-(trifluoromethoxy)phenyl)ethan-1-ol (71 mg, 0.317 mmol), (cyanomethyl)trimethylphosphonium iodide (115 mg, 0.474 mmol) and DIPEA (204 mg, 1.58 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to give the residue, which was purified by Pre-TLC (CH₂Cl₂/MeOH=10:1) to give the titled compound (4 mg). ¹H NMR (400 MHz, CD₃OD) δ 7.47-7.34 (m, 1H), 7.23-7.07 (m, 2H), 6.32-4.99 (m, 2H), 4.27-4.06 (m, 3H), 3.93 (s, 3H), 3.78 (s, 3H), 3.62-3.54 (m, 1H), 2.94-2.71 (m, 2H), 2.15-2.04 (m, 1H), 1.54-1.18 (m, 6H), 1.04-0.89 (m, 3H) ppm. MS: M/e 522 (M+1)⁺.

### Compound A27: 2-(6-((2S,5R)-4-(1-(4-fluoro-2-(methoxymethyl)phenyl)ethyl)-2,5-dimethylpiperazin-1-yl)-3,9-dimethyl-2-oxo-3,9-dihydro-2H-purin-8-yl)acetonitrile

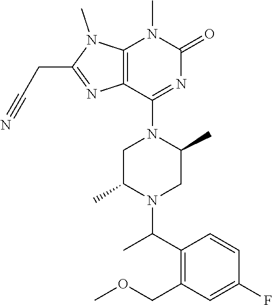 [SMILES: COCc1cc(F)ccc1C(C)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (50 mg, 0.159 mol), 1-(4-fluoro-2-(methoxymethyl)phenyl)ethan-1-ol (44 mg, 0.238 mol), (cyanomethyl)trimethylphosphonium iodide (77 mg. 0.318 mol) and DIPEA (62 mg, 0.477 mol) in CH₃CN (1 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) to give the titled Compound A27, which was further separated into Compound A27a (2 mg) and Compound A27b (1.5 mg) by Prep-HPLC (Method A).

Compound A27a (the earlier peak): ¹H NMR (400 MHz, CD₃OD) δ 7.74-7.64 (m, 1H), 7.04 (dd, J=13.8, 5.8 Hz, 2H), 6.00-5.89 (m, 1H), 5.02-4.96 (m, 0.5H), 4.83-4.71 (m, 0.5H), 4.68-4.38 (m, 2H), 4.21 (s, 1H), 3.95-3.91 (m, 3H), 3.82-3.75 (m, 4H), 3.67-3.62 (m, 2H), 3.37 (s, 3H), 2.96-2.56 (m, 2H), 2.12 (d, J=11.6 Hz, 1H), 1.30 (d, J=6.5 Hz, 3H), 1.24 (d, J=6.3 Hz, 3H), 1.05 (d, J=6.5 Hz, 3H) ppm. MS: M/e 482 (M+1)⁺.

Compound A27b (the later peak): ¹H NMR (400 MHz, CD₃OD) δ 7.61 (dd, J=8.6, 5.9 Hz, 1H), 7.11 (dd, J=9.9, 2.6 Hz, 1H), 7.04 (dd, J=9.7, 7.1 Hz, 1H), 6.17-6.13 (m, 0.5H), 5.65 (d, J=12.5 Hz, 0.5H), 5.25 (s, 0.5H), 4.63-4.51 (m, 2.5H), 4.22-4.17 (m, 1H), 4.06-3.91 (m, 4H), 3.78 (s, 3H), 3.55-3.49 (m, 1H), 3.40 (s, 3H), 3.15-3.10 (m, 1H), 2.92-2.75 (m, 3H), 1.45 (d, J=17.2 Hz, 3H), 1.27 (d, J=6.4 Hz, 3H), 0.88 (d, J=6.5 Hz, 3H) ppm. MS: M/e 482 (M+1)⁺.

### Compound A28: 2-(6-((2S,5R)-4-(1-(4-fluoro-2-(1-methoxyethyl)phenyl)ethyl)-2,5-dimethylpiperazin-1-yl)-3,9-dimethyl-2-oxo-3,9-dihydro-2H-purin-8-yl)acetonitrile

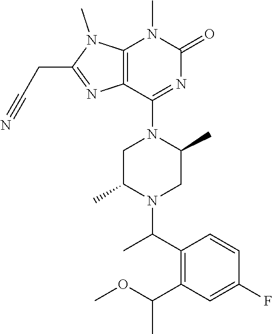 [SMILES: COC(C)c1cc(F)ccc1C(C)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (50 mg, 0.16 mmol), 1-(4-fluoro-2-(1-methoxyethyl)phenyl)ethan-1-ol (31 mg, 0.16 mmol), (cyanomethyl)trimethylphosphonium iodide (115 mg, 0.48 mmol) and DIPEA (102 mg, 0.79 mmol) in MeCN (3 ml) was stirred at 100° C. overnight. After completed, the solution was concentrated under reduced pressure to dryness. The resulting residue was purified by Prep-HPLC (Method A) to give the titled compound (12 mg, 15%). ¹H NMR (400 MHz, CD₃OD) δ 7.74-7.59 (m, 1H), 7.16-6.96 (m, 2H), 6.00-4.80 (m, 3H), 4.57 (s, 3H), 3.93 (s, 3H), 3.78 (s, 3H), 3.60-3.22 (m, 2H), 2.96-2.70 (m, 4H), 2.23-1.99 (m, 1H), 1.60-1.41 (m, 6H), 1.26-1.03 (m, 3H), 0.90 (t, J=7.5 Hz, 3H) ppm. MS: M/e 496 (M+1)⁺

### Compound A29: 2-(6-((2S,5R)-4-(1-(2-(1-(difluoromethoxy)ethyl)-4-fluorophenyl)ethyl)-2,5-dimethylpiperazin-1-yl)-3,9-dimethyl-2-oxo-3,9-dihydro-2H-purin-8-yl)acetonitrile

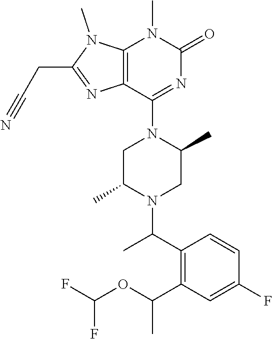 [SMILES: CC(OC(F)F)c1cc(F)ccc1C(C)N1C[C@H](C)N(c2nc(=O)n(C)c3c2nc(CC#N)n3C)C[C@H]1C]

### Step A: 1-bromo-2-(1-(difluoromethoxy)ethyl)-4-fluorobenzene

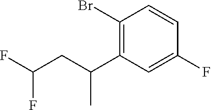

To a solution of 1-(2-bromo-5-fluorophenyl)ethan-1-ol (657 mg, 3 mmol) in DCM (11.8 ml) and H₂O (1.8 ml) were added (bromodifluoromethyl)trimethylsilane (1.8 g, 9 mmol) and AcOK (1.79 g, 18 mmol). The mixture was stirred at RT for 4 hours. The reaction was diluted with DCM and washed with water. The organic layer was separated, dried by Na₂SO₄, filtered, and concentrated to dryness. The resulting residue was purified by flash column chromatography to give the titled compound (260 mg, 32%). MS: M/e 269 (M+1)⁺.

### Step B: 1-(2-(1-(difluoromethoxy)ethyl)-4-fluorophenyl)ethan-1-one

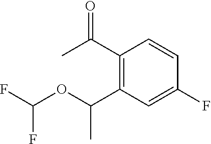 [SMILES: CC(=O)c1ccc(F)cc1C(C)OC(F)F]

To a solution of 1-bromo-2-(1-(difluoromethoxy)ethyl)-4-fluorobenzene (268 mg, 1 mol) in toluene (10 mL) was added tributyl(1-ethoxyvinyl)stannane (430 mg, 1.2 mol) and Pd(PPh₃)₄ (140 mg, 0.2 mmol). The reaction mixture was protected by N₂ atmosphere and stirred at 100° C. for overnight. The mixture was cooled down to rt, added HCl/Dioxane (4M, 10 ml) and stirred at room temperature for 30 mins. The mixture was concentrated in vacuo. The residue was added H₂O and adjusted pH 7-8 by NaHCO₃ aqueous solution. The resulting mixture was extracted with EtOAc, and then concentrated by using a rotary evaporator, to give a residue. The resulting residue was purified by flash column chromatography to give the titled compound (190 mg, 82%). MS: M/e 233 (M+1)⁺.

### Step C: 1-(2-(1-(difluoromethoxy)ethyl)-4-fluorophenyl)ethan-1-ol

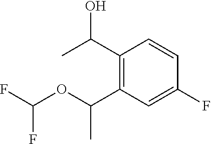 [SMILES: CC(O)c1ccc(F)cc1C(C)OC(F)F]

NaBH₄ (38 mg, 1 mmol) was added to 1-(2-(1-(difluoromethoxy)ethyl)-4-fluorophenyl)ethan-1-one (190 mg, 0.8 mol) in EtOH (200 ml) at 0° C. for 1 hour. The reaction was quenched by adding water. The mixture was extracted with EtOAc and washed with brine. The organic layer was separated, dried by Na₂SO₄, filtered and concentrated to dryness. The resulting residue was purified by flash column chromatography to give the titled compound (50 mg, 26%). MS: M/e 235 (M+1)⁺.

### Step D: 2-(6-((2S,5R)-4-(1-(2-(1-(difluoromethoxy)ethyl)-4-fluorophenyl)ethyl)-2,5-dimethylpiperazin-1-yl)-3,9-dimethyl-2-oxo-3,9-dihydro-2H-purin-8-yl)acetonitrile

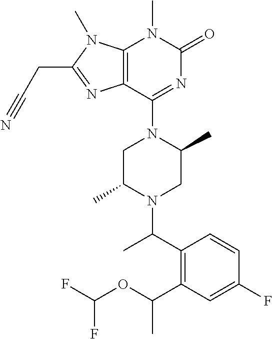 [SMILES: CC(OC(F)F)c1cc(F)ccc1C(C)N1C[C@H](C)N(c2nc(=O)n(C)c3c2nc(CC#N)n3C)C[C@H]1C]

To a solution of 2-(6-((2S,5R)-2,5-dimethylpiperazin-1-yl)-3,9-dimethyl-2-oxo-3,9-dihydro-2H-purin-8-yl)acetonitrile (40 mg, 0.12 mmol) and (cyanomethyl)trimethylphosphonium iodide (120 mg, 1 mmol) in CH₃CN (5 mL) was added DIPEA (250 mg, 2 mmol). The mixture solution was degassed 3 times under N₂ atmosphere. Then the mixture solution was stirred at 105° C. for 24 hours. The reaction was quenched with saturated NH₄Cl (20 mL) at room temperature. The resulting mixture was extracted with EtOAc (35 mL×2). The combined organic layers were dried over Na₂SO₄ and concentrated under reduced pressure. The resulting residue was purified by Prep-HPLC(Method A) to give the titled Compound A29a (0.51 mg) and Compound A29b (0.83 mg).

Compound A29a (the earlier peak): ¹H NMR (400 MHz, CD₃OD) δ 7.72 (dd, J=8.8, 6.0 Hz, 1H), 7.14 (dd, J=10.3, 2.7 Hz, 1H), 7.04 (dd, J=8.5, 5.6 Hz, 1H), 6.55-6.17 (m, 1H), 5.66 (d, J=6.4 Hz, 1H), 5.34 (s, 1H), 4.56 (s, 2H), 3.94 (s, 3H), 3.84 (d, J=6.5 Hz, 1H), 3.78 (s, 3H), 3.59 (s, 1H), 2.77 (s, 1H), 2.68 (d, J=12.3 Hz, 1H), 2.21-2.15 (m, 1H), 2.03 (m, 1H), 1.50 (d, J=6.5 Hz, 3H), 1.30 (s, 3H), 1.22 (d, J=6.6 Hz, 3H), 1.07 (d, J=6.6 Hz, 3H) ppm. MS: M/e 532 (M+1)⁺.

Compound A29b (the later peak): ¹H NMR (400 MHz, CD₃OD) δ 7.62-7.54 (m, 1H), 7.21 (dd, J=10.4, 2.7 Hz, 1H), 7.04 (td, J=8.4, 2.6 Hz, 1H), 6.58-6.20 (m, 1H), 5.90 (d, J=6.6 Hz, 1H), 5.65 (d, J=13.2 Hz, 1H), 5.26 (s, 1H), 4.56 (s, 2H), 3.90 (dd, J=21.3, 5.6 Hz, 4H), 3.78 (s, 3H), 3.55-3.48 (m, 1H), 2.84 (s, 2H), 2.03 (m, 1H), 1.51 (d, J=6.3 Hz, 3H), 1.30 (d, J=6.5 Hz, 6H), 0.91 (d, J=6.5 Hz, 3H) ppm. MS: M/e 532 (M+1)⁺.

### Compound A30: 2-(6-((2S,5R)-4-(1-(2-(difluoromethyl)-4-fluorophenyl)ethyl)-2,5-dimethylpiperazin-1-yl)-3,9-dimethyl-2-oxo-3,9-dihydro-2H-purin-8-yl)acetonitrile

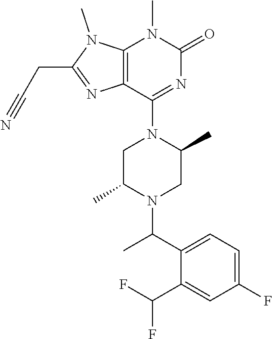 [SMILES: CC(c1ccc(F)cc1C(F)F)N1C[C@H](C)N(c2nc(=O)n(C)c3c2nc(CC#N)n3C)C[C@H]1C]

### Step A: 1-(2-(difluoromethyl)-4-fluorophenyl)ethan-1-one

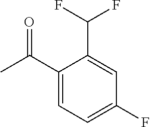

A mixture of 1-bromo-2-(difluoromethyl)-4-fluorobenzene (1.12 g, 5 mmol), tributyl(1-ethoxyvinyl)stannane (2.17 g, 6 mmol) and Pd(PPh₃)₂Cl₂ (350 mg, 0.5 mmol) in toluene (10 mL) was stirred at 100° C. under N₂ overnight. Then to the solution was added HCl (4 mL, 4 M in 1,4-dioxane) in drops and the mixture was stirred at room temperature for 0.5 hour. The reaction mixture was diluted with EtOAc (50 mL), treated with saturated NaHCO₃ aq. to pH˜8, washed with brine (20 mL×3), dried over Na₂SO₄ and concentrated to dryness. The resulting residue was purified by flash column chromatography to give the titled compound (800 mg, 85%). ¹H NMR (400 MHz, CD₃OD) δ 8.10 (dd, J=8.2, 5.9 Hz, 1H), 7.54-7.18 (m, 3H), 2.61 (d, J=6.9 Hz, 3H) ppm. MS: M/e 189 (M+1)⁺.

### Step B: 1-(2-(difluoromethyl)-4-fluorophenyl)ethan-1-ol

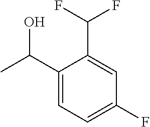

To a solution of 1-(2-(difluoromethyl)-4-fluorophenyl)ethan-1-one (400 mg, 2.13 mmol) in MeOH (5 mL) was added NaBH₄ (65 mg, 1.7 mmol) at ° C. and the resulting mixture was stirred at room temperature for 1 hour. The resulting residue was treated with water, extracted with EtOAc (20 mL×3). The combined organic layers were washed with brine (20 mL×3), dried over Na₂SO₄ and concentrated to dryness. The resulting residue was purified by flash column chromatography (EtOAc:PE=0-100% in 20 minutes) to give the titled compound (190 mg, 47%). ¹H NMR (400 MHz, DMSO-d6) δ 7.70-7.62 (m, 1H), 7.51-7.19 (m, 3H), 5.44 (d, J=4.0 Hz, 1H), 5.10-5.02 (m, 1H), 1.32 (d, J=6.4 Hz, 3H) ppm. MS: M/e 191 (M+1)⁺.

### Step C: 2-(6-((2S,5R)-4-(1-(2-(difluoromethyl)-4-fluorophenyl)ethyl)-2,5-dimethylpiperazin-1-yl)-3,9-dimethyl-2-oxo-3,9-dihydro-2H-purin-8-yl)acetonitrile

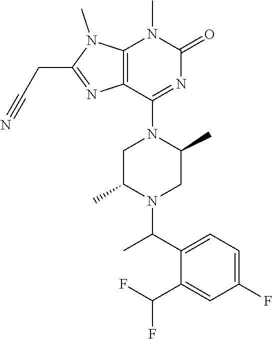 [SMILES: CC(c1ccc(F)cc1C(F)F)N1C[C@H](C)N(c2nc(=O)n(C)c3c2nc(CC#N)n3C)C[C@H]1C]

A mixture of 1-(2-(difluoromethyl)-4-fluorophenyl)ethan-1-ol (57 mg, 0.3 mmol), 2-(6-((2S,5R)-2,5-dimethylpiperazin-1-yl)-3,9-dimethyl-2-oxo-3, 9-dihydro-2H-purin-8-yl)acetonitrile (63 mg, 0.2 mmol), (cyanomethyl)trimethylphosphonium iodide (97 mg, 0.4 mmol) and DIPEA (103 mg, 0.8 mmol) in MeCN (2 mL) was stirred at 100° C. for overnight in a sealed tube. The mixture was treated with water, extracted with EtOAc. The combined organic layers were washed with brine (20 mL×2), dried over Na₂SO₄, concentrated to dryness. The resulting residue was purified by Prep-TLC (CH₂Cl₂:MeOH=13:1) to give the titled compound (5 mg, 5%). ¹H NMR (400 MHz, CD₃OD) δ 7.81-7.10 (m, 4H), 6.29-5.22 (m, 2H), 4.22 (t, J=13.1 Hz, 1H), 3.93 (d, J=6.6 Hz, 3H), 3.86-3.76 (m, 3H), 3.67-3.50 (m, 1H), 3.35-3.20 (m, 2H), 2.92-2.69 (m, 2H), 2.09-1.98 (m, 1H), 1.48-1.22 (m, 6H), 1.05-0.87 (m, 3H) ppm. MS: M/e 488 (M+1)⁺.

### Compound A31: 2-(6-((2S,5R)-4-(1-(2-(1,1-difluoroethoxy)-4-fluorophenyl)ethyl)-2,5-dimethylpiperazin-1-yl)-3,9-dimethyl-2-oxo-3,9-dihydro-2H-purin-8-yl)acetonitrile

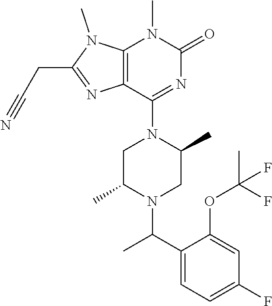 [SMILES: CC(c1ccc(F)cc1OC(C)(F)F)N1C[C@H](C)N(c2nc(=O)n(C)c3c2nc(CC#N)n3C)C[C@H]1C]

A mixture of 2-(6-((2S,5R)-2,5-dimethylpiperazin-1-yl)-3,9-dimethyl-2-oxo-3,9-dihydro-2H-purin-8-yl)acetonitrile (50 mg, 0.158 mmol), 1-(2-(1,1-difluoroethoxy)-4-fluorophenyl)ethan-1-ol (64 mg, 0.317 mmol), (cyanomethyl)trimethylphosphonium iodide (115 mg, 0.474 mmol) and DIPEA (204 mg, 1.58 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to give the residue, which was purified by Pre-TLC (CH₂Cl₂/MeOH=10:1) to the desired compound, which was further purified by Prep-HPLC (Method A) to give the titled compound (0.5 mg). ¹H NMR (400 MHz, CD₃OD) δ 8.06-7.95 (m, 1H), 7.28-7.16 (m, 2H), 6.21-5.21 (m, 2H), 4.28-4.14 (m, 1H), 4.03-3.97 (m, 1H), 3.96-3.90 (m, 3H), 3.78 (s, 3H), 3.63 (s, 1H), 2.98-2.66 (m, 4H), 2.08-1.91 (m, 3H), 1.56-1.28 (m, 3H), 1.28-1.18 (m, 3H), 1.08-0.88 (m, 3H) ppm. MS: M/e 518 (M+1)⁺.

### Compound A33: 2-(6-((2S,5R)-4-(1-(4-fluoro-2-(trifluoromethyl)phenyl)ethyl)-2,5-dimethylpiperazin-1-yl)-3,9-dimethyl-2-oxo-3,9-dihydro-2H-purin-8-yl)acetonitrile

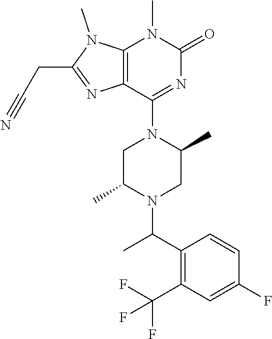 [SMILES: CC(c1ccc(F)cc1C(F)(F)F)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (50 mg, 0.159 mol), 1-(4-fluoro-2-(trifluoromethyl)phenyl)ethan-1-ol (66 mg, 0.317 mol), (cyanomethyl)trimethylphosphonium iodide (77 mg. 0.317 mol) and DIPEA (62 mg, 0.477 mol) in CH₃CN (1 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) to give the titled Compound A33, which was further separated into Compound A33a (4 mg) and Compound A33b (3 mg) by Prep-HPLC (Method A).

Compound A33a (the earlier peak): ¹H NMR (400 MHz, CD₃OD) δ 8.10-8.00 (m, 1H), 7.40 (t, J=8.6 Hz, 2H), 5.94-5.89 (m, 1H), 5.07-5.03 (m, 0.5H), 4.86-4.82 (m, 0.5H), 4.23-4.18 (m, 1.5H), 3.93 (s, 3H), 3.92-3.87 (m, 1H), 3.79-3.74 (m, 3.5H), 3.64 (s, 1H), 3.46-3.42 (m, 0.5H), 2.73-2.64 (m, 1.5H), 1.99 (d, J=12.2 Hz, 1H), 1.32 (d, J=6.3 Hz, 3H), 1.23 (dd, J=17.0, 6.3 Hz, 3H), 1.04 (d, J=6.4 Hz, 3H) ppm. MS: M/e 506 (M+1)⁺.

Compound A33b (the later peak): ¹H NMR (400 MHz, CD₃OD) δ 8.18-8.05 (m, 1H), 7.44 (t, J=8.8 Hz, 2H), 6.23-6.18 (m, 0.5H), 5.66 (d, J=13.3 Hz, 0.5H), 5.29 (s, 0.5H), 4.60 (d, J=13.1 Hz, 0.5H), 4.08-4.02 (m, 1H), 3.95 (d, J=14.6 Hz, 3H), 3.83-3.76 (m, 3.5H), 3.57-3.52 (m, 0.5H), 3.35-3.31 (m, 1.5H), 3.25-3.20 (m, 0.5H), 2.97-2.84 (m, 2H), 2.83-2.77 (m, 1H), 1.52 (dd, J=26.0, 6.6 Hz, 3H), 1.30-1.21 (m, 3H), 0.99-0.83 (m, 3H) ppm. MS: M/e 506 (M+1)⁺.

### Compound A34: 2-(6-((2S,5R)-2,5-diethyl-4-(1-(4-fluoro-2-(trifluoromethyl)phenyl)ethyl) piperazin-1-yl)-3,9-dimethyl-2-oxo-3,9-dihydro-2H-purin-8-yl)acetonitrile

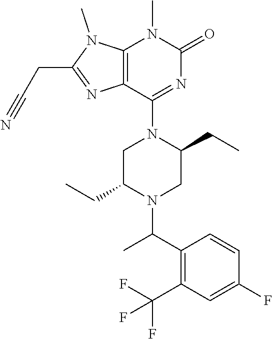 [SMILES: CC[C@H]1CN(C(C)c2ccc(F)cc2C(F)(F)F)[C@H](CC)CN1c1nc(=O)n(C)c2c1nc(CC#N)n2C]

### Step A: 2-chloro-6-((2S,5R)-2,5-diethyl-4-(1-(4-fluoro-2-(trifluoromethyl)phenyl)ethyl) piperazin-1-yl)-9-methyl-9H-purine

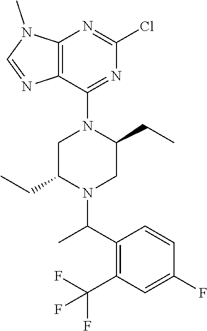 [SMILES: CC[C@H]1CN(C(C)c2ccc(F)cc2C(F)(F)F)[C@H](CC)CN1c1nc(Cl)nc2c1ncn2C]

To a solution of 2,6-dichloro-9-methyl-9H-purine (311 mg, 1.54 mmol) and (2R,5S)-2,5-diethyl-1-(1-(4-fluoro-2-(trifluoromethyl)phenyl)ethyl)piperazine (450 mg, 1.40 mmol) in n-BuOH (20 mL) was added DIPEA (542 mg, 4.20 mmol). The reaction was stirred at room temperature overnight. The reaction mixture was concentrated under reduced pressure to give the title compound (650 mg, 84%). MS: M/e 499 (M+1)⁺.

### Step B: 6-((2S,5R)-2,5-diethyl-4-(1-(4-fluoro-2-(trifluoromethyl)phenyl)ethyl)piperazin-1-yl)-9-methyl-9H-purin-2-ol

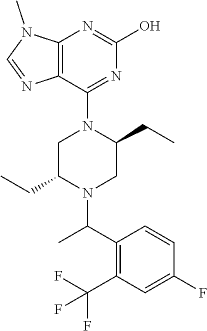 [SMILES: CC[C@H]1CN(C(C)c2ccc(F)cc2C(F)(F)F)[C@H](CC)CN1c1nc(O)nc2c1ncn2C]

To a solution of 2-chloro-6-((2S,5R)-2,5-diethyl-4-(1-(4-fluoro-2-(trifluoromethyl)phenyl)ethyl)piperazin-1-yl)-9-methyl-9H-purine (600 mg, 1.204 mmol), Pd₂(dba)₃ (55 mg, 0.06 mmol), t-BuXphos (51 mg, 0.12 mmol) and KOH (1.2 mL, 3M, 3.6 mmol) in dioxane (10 mL) was degassed 3 times under N₂ atmosphere. Then the mixture was stirred at 90° C. for 12 hours. The reaction mixture was diluted with water and extracted with EtOAc (30 mL×2). The combined organic layers were washed with brine, dried over Na₂SO₄, filtered and concentrated to dryness. The resulting residue was purified by flash column chromatography (DCM:MeOH=10:1) to give the titled compound (470 mg, 82%). MS: M/e 481 (M+1)⁺.

### Step C: 6-((2S,5R)-2,5-diethyl-4-(1-(4-fluoro-2-(trifluoromethyl)phenyl)ethyl)piperazin-1-yl)-3,9-dimethyl-3,9-dihydro-2H-purin-2-one

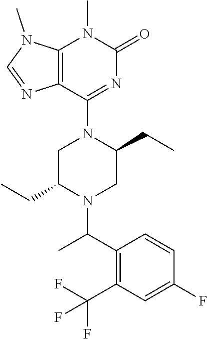 [SMILES: CC[C@H]1CN(C(C)c2ccc(F)cc2C(F)(F)F)[C@H](CC)CN1c1nc(=O)n(C)c2c1ncn2C]

To a solution of 6-((2S,5R)-2,5-diethyl-4-(1-(4-fluoro-2-(trifluoromethyl)phenyl)ethyl)piperazin-1-yl)-9-methyl-9H-purin-2-ol (470 mg, 0.979 mmol) and Cs₂CO₃ (638 mg, 1.958 mmol) in dioxane (30 mL) was added CH₃I (208 mg, 1.468 mmol). The reaction was stirred at room temperature overnight. The reaction mixture was diluted with water, extracted with EtOAc (60 mL×2), washed with brine, dried over Na₂SO₄, filtered, and concentrated to dryness. The resulting residue was purified by flash column chromatography (DCM:MeOH=10:1) to give the titled compound (270 mg, 56%). MS: M/e 495 (M+1)⁺.

### Step D: 6-((2S,5R)-2,5-diethyl-4-(1-(4-fluoro-2-(trifluoromethyl)phenyl)ethyl)piperazin-1-yl)-3,9-dimethyl-2-oxo-3,9-dihydro-2H-purine-8-carbaldehyde

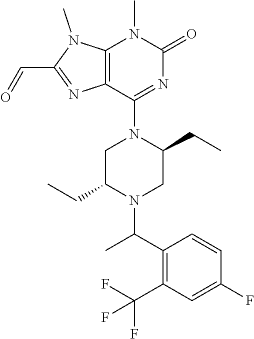 [SMILES: CC[C@H]1CN(C(C)c2ccc(F)cc2C(F)(F)F)[C@H](CC)CN1c1nc(=O)n(C)c2c1nc(C=O)n2C]

To a solution of 6-((2S,5R)-2,5-diethyl-4-(1-(4-fluoro-2-(trifluoromethyl)phenyl)ethyl)piperazin-1-yl)-3,9-dimethyl-3,9-dihydro-2H-purin-2-one (240 mg, 0.485 mmol) in THE (15 mL) was degassed 3 times under N₂ atmosphere. Then added LDA (0.97 mL, 2M, 1.940 mmol) at −78° C. The mixture was stirred at −78° C. for 1 hour, DMF (106 mg, 1.455 mmol) was added. The reaction solution was stirred to room temperature for 2 hours. The reaction mixture was diluted with water, extracted with EtOAc (45 mL×2), washed with brine, dried over Na₂SO₄, filtered, and concentrated to dryness. The resulting residue was purified by flash column chromatography (DCM:MeOH=10:1) to give the titled compound (180 mg, 71%). MS: M/e 523 (M+1)⁺.

### Step E: 6-((2S,5R)-2,5-diethyl-4-(1-(4-fluoro-2-(trifluoromethyl)phenyl)ethyl)piperazin-1-yl)-8-(hydroxymethyl)-3,9-dimethyl-3,9-dihydro-2H-purin-2-one

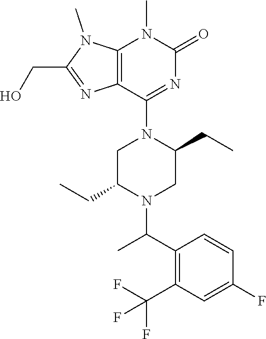 [SMILES: CC[C@H]1CN(C(C)c2ccc(F)cc2C(F)(F)F)[C@H](CC)CN1c1nc(=O)n(C)c2c1nc(CO)n2C]

To a solution of 6-((2S,5R)-2,5-diethyl-4-(1-(4-fluoro-2-(trifluoromethyl)phenyl)ethyl)piperazin-1-yl)-3,9-dimethyl-2-oxo-3, 9-dihydro-2H-purine-8-carbaldehyde (180 mg, 0.345 mmol) in MeOH (10 mL) was added NaBH₄ (20 mg, 0.517 mmol). The reaction was stirred at room temperature for 1 hour. The reaction mixture was quenched with saturated NH₄Cl, extracted with EtOAc (30 mL×2), washed with brine, dried over Na₂SO₄, filtered, and concentrated to dryness. The resulting residue was purified by flash column chromatography (DCM:MeOH=8:1) to give the titled compound (150 mg, 83%). MS: M/e 525 (M+1)⁺.

### Step F: 8-(chloromethyl)-6-((2S,5R)-2,5-diethyl-4-(1-(4-fluoro-2-(trifluoromethyl)phenyl)ethyl) piperazin-1-yl)-3,9-dimethyl-3,9-dihydro-2H-purin-2-one

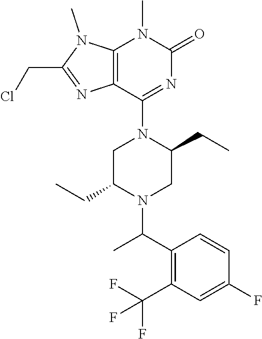 [SMILES: CC[C@H]1CN(C(C)c2ccc(F)cc2C(F)(F)F)[C@H](CC)CN1c1nc(=O)n(C)c2c1nc(CCl)n2C]

To a solution of 6-((2S,5R)-2,5-diethyl-4-(1-(4-fluoro-2-(trifluoromethyl)phenyl)ethyl)piperazin-1-yl)-8-(hydroxymethyl)-3,9-dimethyl-3,9-dihydro-2H-purin-2-one (150 mg, 0.286 mmol) in DCM (5 mL) was added SOCl₂ (136 mg, 1.145 mmol). The reaction was stirred at room temperature for 0.5 hours. The reaction mixture concentrated to give the titled compound (150 mg, 97%). MS: M/e 543 (M+1)⁺.

### Step G: 2-(6-((2S,5R)-2,5-diethyl-4-(1-(4-fluoro-2-(trifluoromethyl)phenyl)ethyl)piperazin-1-yl)-3,9-dimethyl-2-oxo-3,9-dihydro-2H-purin-8-yl)acetonitrile

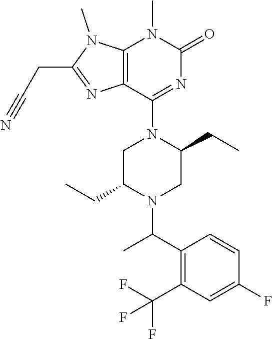 [SMILES: CC[C@H]1CN(C(C)c2ccc(F)cc2C(F)(F)F)[C@H](CC)CN1c1nc(=O)n(C)c2c1nc(CC#N)n2C]

To a solution of 8-(chloromethyl)-6-((2S,5R)-2,5-diethyl-4-(1-(4-fluoro-2-(trifluoromethyl)phenyl)ethyl)piperazin-1-yl)-3,9-dimethyl-3,9-dihydro-2H-purin-2-one (150 mg, 0.277 mmol) and Cs₂CO₃ (361 mg, 1.107 mmol) in CH₃CN (10 mL) was added TMSCN (110 mg, 1.107 mmol). The reaction was stirred at room temperature for 4 hours. The reaction mixture was diluted with water, extracted with DCM (30 mL×2), washed with brine, dried over Na₂SO₄, filtered, and concentrated to dryness. The resulting residue was purified by Prep-TLC (DCM:MeOH=15:1) to give the titled Compound A34, which was further separated into Compound A34a (19 mg) and Compound A34b (10 mg) by Prep-HPLC (Method A).

Compound A34a (the earlier peak): ¹H NMR (400 MHz, CD₃OD) 8.09-7.96 (m, 1H), 7.40 (t, J=7.9 Hz, 2H), 6.14 (d, J=13.7 Hz, 0.5H), 5.78-5.74 (m, 0.5H), 5.12 (d, J=13.2 Hz, 0.5H), 4.91-4.87 (m, 0.5H), 4.21 (d, J=24.8 Hz, 2H), 4.02-3.93 (m, 1H), 3.92 (d, J=4.3 Hz, 3H), 3.79 (t, J=3.9 Hz, 3H), 3.62 (d, J=13.7 Hz, 0.5H), 3.25-3.19 (m, 1.5H), 2.68-2.51 (m, 1H), 2.11 (d, J=12.2 Hz, 1H), 1.84-1.66 (m, 2H), 1.54-1.48 (m, 2H), 1.28 (d, J=6.4 Hz, 3H), 1.04-0.99 (m, 3H), 0.66-0.61 (m, 3H) ppm. MS: M/e 534 (M+1)⁺.

Compound A34b (the later peak): ¹H NMR (400 MHz, CD₃OD) δ 8.06 (dd, J=8.2, 6.0 Hz, 1H), 7.46-7.40 (m, 2H), 6.05-6.01 (m, 0.5H), 5.88 (d, J=13.5 Hz, 0.5H), 5.18-5.13 (m, 0.5H), 4.87-4.83 (m, 0.5H), 4.30-4.09 (m, 3H), 3.92 (d, J=10.3 Hz, 3H), 3.78 (d, J=2.1 Hz, 3H), 3.37 (d, J=11.7 Hz, 0.5H), 3.08-2.96 (m, 1.5H), 2.88-2.71 (m, 1H), 2.28 (t, J=10.4 Hz, 1H), 2.18-1.96 (m, 2H), 1.51-1.35 (m, 2H), 1.26 (dd, J=6.2, 2.4 Hz, 3H), 0.99-0.93 (m, 3H), 0.79-0.64 (m, 3H) ppm. MS: M/e 534 (M+1)⁺.

### Compound A35: 2-(9-ethyl-6-((2S,5R)-4-(1-(4-fluoro-2-(trifluoromethyl)phenyl)ethyl)-2,5-dimethylpiperazin-1-yl)-3-methyl-2-oxo-3,9-dihydro-2H-purin-8-yl)acetonitrile

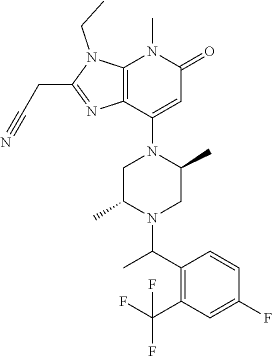 [SMILES: CCn1c(CC#N)nc2c(N3C[C@@H](C)N(C(C)c4ccc(F)cc4C(F)(F)F)C[C@@H]3C)cc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol), 1-(4-fluoro-2-(trifluoromethyl)phenyl)ethan-1-ol (32 mg, 0.15 mmol), (cyanomethyl)trimethylphosphonium iodide (110 mg, 0.46 mmol) and DIPEA (98 mg, 0.76 mmol) in MeCN (3 ml) was stirred at 100° C. overnight. After completed, the solution was concentrated under reduced pressure. The resulting residue was purified by Prep-HPLC (Method A) to give the titled Compound A35a (16 mg) and Compound A35b (9 mg).

Compound A35a (the earlier peak): ¹H NMR (400 MHz, CD₃OD) δ 8.11-8.00 (m, 1H), 7.39 (t, J=8.9 Hz, 2H), 5.92 (d, J=13.2 Hz, 1H), 5.05 (s, 1H), 4.37 (q, J=7.2 Hz, 2H), 4.26-4.20 (m, 1H), 3.88 (s, 1H), 3.76 (s, 3H), 3.65 (s, 1H), 3.48 (s, 1H), 2.84-2.56 (m, 2H), 1.99 (d, J=12.5 Hz, 1H), 1.46 (t, J=7.2 Hz, 3H), 1.32 (d, J=6.3 Hz, 3H), 1.23 (dd, J=18.9, 6.8 Hz, 3H), 1.06 (d, J=6.6 Hz, 3H) ppm. MS: M/e 520 (M+1)⁺

Compound A35b (the later peak): ¹H NMR (400 MHz, CD₃OD) δ 8.16-8.05 (m, 1H), 7.42 (t, J=9.0 Hz, 2H), 6.34-5.04 (m, 2H), 4.37 (dd, J=15.2, 7.5 Hz, 2H), 4.05 (s, 1H), 3.76 (s, 3H), 3.61-3.36 (m, 1H), 3.26-3.10 (m, 1H), 2.99-2.88 (m, 2H), 2.78 (s, 2H), 1.57-1.41 (m, 6H), 1.25 (d, J=5.8 Hz, 3H), 0.90 (t, J=6.7 Hz, 3H) ppm. MS: M/e 520 (M+1)⁺

### Compound A36: 2-(6-((2S,5R)-4-(1-(4-fluoro-2-(trifluoromethyl)phenyl)ethyl)-2,5-dimethylpiperazin-1-yl)-3,9-bis(methyl-d3)-2-oxo-3,9-dihydro-2H-purin-8-yl)acetonitrile

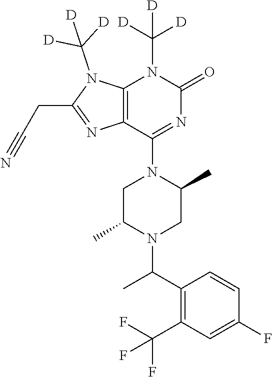 [SMILES: [2H]C([2H])([2H])n1c(CC#N)nc2c(N3C[C@@H](C)N(C(C)c4ccc(F)cc4C(F)(F)F)C[C@@H]3C)nc(=O)n(C([2H])([2H])[2H])c21]

### Step A: 2,6-dichloro-9-(methyl-d3)-9H-purine

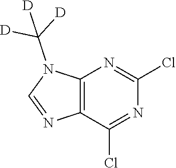 [SMILES: [2H]C([2H])([2H])n1cnc2c(Cl)nc(Cl)nc21]

To a stirred solution of 2,6-dichloro-9H-purine (3.78 g, 20 mmol) in DMF (30 mL) was added K₂CO₃ (5.52 g, 40 mmol), followed by CD3I (4.35 g, 30 mmol). After the addition, the reaction mixture was stirred overnight. The reaction mixture was treated with H₂O (50 mL) and extracted with CH₂Cl₂ (50 mL×3). The combined organic layers were washed with brine, dried over Na₂SO₄, concentrated and purified by flash column chromatography to give the titled compound (2.52 g, 61%). MS: M/e 206 (M+1)⁺.

### Step B: tert-butyl (2R,5S)-4-(2-chloro-9-(methyl-d3)-9H-purin-6-yl)-2,5-dimethylpiperazine-1-carboxylate

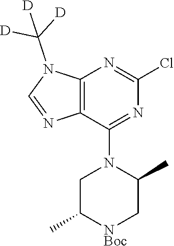 [SMILES: [2H]C([2H])([2H])n1cnc2c(N3C[C@@H](C)N(C(=O)OC(C)(C)C)C[C@@H]3C)nc(Cl)nc21]

A mixture of 2,6-dichloro-9-(methyl-d3)-9H-purine (2.52 g, 12.2 mmol), tert-butyl (2R,5S)-2,5-dimethylpiperazine-1-carboxylate (2.61 g, 12.2 mmol) and DIPEA (2.37 g, 18.3 mmol) in n-BuOH (25 mL) was stirred for 3 days. The reaction mixture was concentrated to give the titled (4.3 g, 92%). MS: M/e 384 (M+1)⁺.

### Step C: tert-butyl (2R,5S)-4-(2-hydroxy-9-(methyl-d3)-9H-purin-6-yl)-2,5-dimethylpiperazine-1-carboxylate

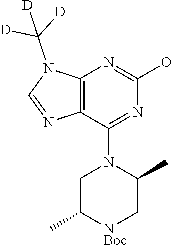 [SMILES: [2H]C([2H])([2H])n1cnc2c(N3C[C@@H](C)N(C(=O)OC(C)(C)C)C[C@@H]3C)nc(O)nc21]

To a stirred solution of tert-butyl (2R,5S)-4-(2-chloro-9-(methyl-d3)-9H-purin-6-yl)-2,5-dimethylpiperazine-1-carboxylate (3.51 g, 9.2 mmol) in dioxane (50 mL) was added Pd₂(dba)₃ (390 mg, 0.92 mmol), t-BuXPhos (390 mg, 0.92 mmol) and aq.KOH (1.55 g, 27.6 mmol in 5 mL H₂O). After the addition, the reaction mixture was stirred at 100° C. for 3 hours under N₂. The mixture was poured into H₂O (100 mL), extracted with EtOAc (50 mL×2). The combined organic layers were discarded and the aqueous layer was acidified to pH=3˜4 with aq.citric acid, then extracted with CH₂Cl₂ (50 mL×3). The combined organic layers were washed with brine, dried over Na₂SO₄, concentrated to give the titled compound (4.15 g, 100%). MS: M/e 366 (M+1)⁺.

### Step D: tert-butyl (2R,5S)-4-(3,9-bis(methyl-d3)-2-oxo-3,9-dihydro-2H-purin-6-yl)-2,5-dimethylpiperazine-1-carboxylate

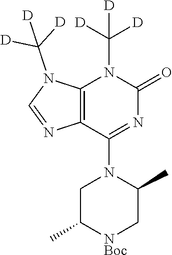 [SMILES: [2H]C([2H])([2H])n1cnc2c(N3C[C@@H](C)N(C(=O)OC(C)(C)C)C[C@@H]3C)nc(=O)n(C([2H])([2H])[2H])c21]

To a stirred mixture of tert-butyl (2R,5S)-4-(2-hydroxy-9-(methyl-d3)-9H-purin-6-yl)-2,5-dimethylpiperazine-1-carboxylate (2 g, 5.46 mmol) in dioxane (50 mL) was added Cs₂CO₃ (5.3 g, 16.38 mmol), followed by CD3I (1.58 g, 10.9 mmol). After then, the mixture was stirred overnight at 80° C. in a sealed tube. The mixture was poured into H₂O (150 mL), extracted with EtOAc (50 mL×3). The combined organic layers were washed with brine, dried over Na₂SO₄, concentrated to dryness. The resulting residue was purified by flash column chromatography to give the titled compound (1 g, 48%). MS: M/e 383 (M+1)⁺.

### Step E: tert-butyl (2R,5S)-4-(8-formyl-3,9-bis(methyl-d3)-2-oxo-3,9-dihydro-2H-purin-6-yl)-2,5-dimethylpiperazine-1-carboxylate

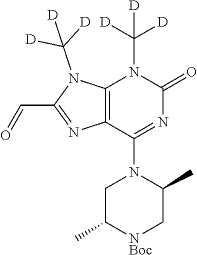 [SMILES: [2H]C([2H])([2H])n1c(C=O)nc2c(N3C[C@@H](C)N(C(=O)OC(C)(C)C)C[C@@H]3C)nc(=O)n(C([2H])([2H])[2H])c21]

To a stirred solution of tert-butyl (2R,5S)-4-(3,9-bis(methyl-d3)-2-oxo-3,9-dihydro-2H-purin-6-yl)-2,5-dimethylpiperazine-1-carboxylate (500 mg, 1.3 mmol) in THE (10 mL) was added LDA (2.0 M, 1.73 mL, 5.2 mmol) dropwise at −78° C. After stirred for an hour, DMF (379 mg, 5.2 mmol) was added and the mixture was allowed warm to room temperature and stirred for an hour. The mixture was quenched with aq.NH₄Cl, extracted with EtOAc (10 mL×3). The combined organic layers were washed with brine, dried over Na₂SO₄, concentrated and directly used to the next step without further purification. MS: M/e 411 (M+1)⁺.

### Step F: tert-butyl (2R,5S)-4-(8-(hydroxymethyl)-3,9-bis(methyl-d3)-2-oxo-3,9-dihydro-2H-purin-6-yl)-2,5-dimethylpiperazine-1-carboxylate

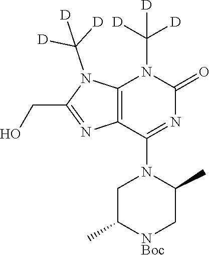 [SMILES: [2H]C([2H])([2H])n1c(CO)nc2c(N3C[C@@H](C)N(C(=O)OC(C)(C)C)C[C@@H]3C)nc(=O)n(C([2H])([2H])[2H])c21]

To a stirred solution of tert-butyl (2R,5S)-4-(8-formyl-3,9-bis(methyl-d3)-2-oxo-3,9-dihydro-2H-purin-6-yl)-2,5-dimethylpiperazine-1-carboxylate (crude, 1.3 mmol) in MeOH (15 mL) was added NaBH₄ (74.1 mg, 1.96 mmol), then stirred for 10 min. The reaction mixture was poured into H₂O (30 mL), extracted with EtOAc (15 mL×3). The combined organic layers were washed with brine, dried over Na₂SO₄, concentrated and purified by flash column chromatography to give the titled compound (240 mg, 45%). MS: M/e 413 (M+1)⁺.

### Step G: tert-butyl (2R,5S)-4-(8-(chloromethyl)-3,9-bis(methyl-d3)-2-oxo-3,9-dihydro-2H-purin-6-yl)-2,5-dimethylpiperazine-1-carboxylate

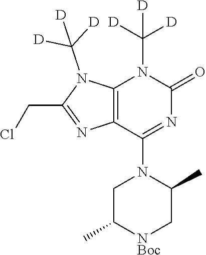 [SMILES: [2H]C([2H])([2H])n1c(CCl)nc2c(N3C[C@@H](C)N(C(=O)OC(C)(C)C)C[C@@H]3C)nc(=O)n(C([2H])([2H])[2H])c21]

To a stirred solution of tert-butyl (2R,5S)-4-(8-(hydroxymethyl)-3,9-bis(methyl-d3)-2-oxo-3,9-dihydro-2H-purin-6-yl)-2,5-dimethylpiperazine-1-carboxylate (240 mg, 0.58 mmol) in CH₂Cl₂ (10 mL) was added SOCl₂ (138 mg, 1.16 mmol). After then, the reaction mixture was stirred for 10 min. The reaction mixture was concentrated to give the titled compound, which was directly used to the next step. MS: M/e 431 (M+1)⁺

### Step H: tert-butyl (2R,5S)-4-(8-(cyanomethyl)-3,9-bis(methyl-d3)-2-oxo-3,9-dihydro-2H-purin-6-yl)-2,5-dimethylpiperazine-1-carboxylate

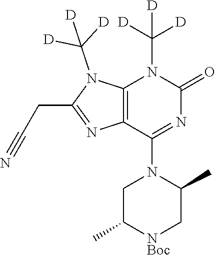 [SMILES: [2H]C([2H])([2H])n1c(CC#N)nc2c(N3C[C@@H](C)N(C(=O)OC(C)(C)C)C[C@@H]3C)nc(=O)n(C([2H])([2H])[2H])c21]

To a stirred solution of tert-butyl (2R,5S)-4-(8-(chloromethyl)-3,9-bis(methyl-d3)-2-oxo-3,9-dihydro-2H-purin-6-yl)-2,5-dimethylpiperazine-1-carboxylate (crude, 0.58 mmol) in CH₂Cl₂ (10 mL) was added TBAF (1.0 M, 2.32 mL, 2.32 mmol), followed by TMSCN (232 mg, 2.32 mmol). After the addition, the reaction mixture was stirred for 5 hours. The reaction mixture was poured into H₂O (30 mL) and extracted with EtOAc (15 mL×3). The combined organic layers were washed with brine, dried over Na₂SO₄, concentrated and purified by flash column chromatography to give the titled compound (crude, 100%). MS: M/e 422 (M+1)⁺

### Step I: 2-(6-((2S,5R)-2,5-dimethylpiperazin-1-yl)-3,9-bis(methyl-d3)-2-oxo-3,9-dihydro-2H-purin-8-yl)acetonitrile

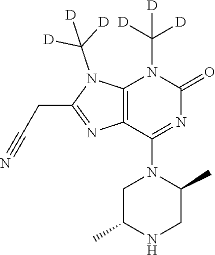 [SMILES: [2H]C([2H])([2H])n1c(CC#N)nc2c(N3C[C@@H](C)NC[C@@H]3C)nc(=O)n(C([2H])([2H])[2H])c21]

To a stirred solution of tert-butyl (2R,5S)-4-(8-(cyanomethyl)-3,9-bis(methyl-d3)-2-oxo-3,9-dihydro-2H-purin-6-yl)-2,5-dimethylpiperazine-1-carboxylate (crude, 0.58 mmol) in CH₂Cl₂ (10 mL) was added TFA (2 mL). After the addition, the reaction mixture was stirred for an hour. The reaction mixture was concentrated to give the residue, which was treated with EtOAc/H₂O (20 mL/20 mL), most TBAF was extracted, and the organic layers were discarded. The aqueous layer was basified to pH=10˜12 with aq.Na₂CO₃ and extracted with CH₂Cl₂/IPA (3/1, 10 mL×4). The combined organic layers were washed with brine, dried over Na₂SO₄, concentrated to give the titled compound (120 mg, 64%). MS: M/e 322 (M+1)⁺

### Step J: 2-(6-((2S,5R)-4-(1-(4-fluoro-2-(trifluoromethyl)phenyl)ethyl)-2,5-dimethylpiperazin-1-yl)-3,9-bis(methyl-d3)-2-oxo-3,9-dihydro-2H-purin-8-yl)acetonitrile

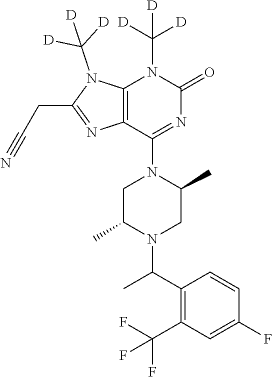 [SMILES: [2H]C([2H])([2H])n1c(CC#N)nc2c(N3C[C@@H](C)N(C(C)c4ccc(F)cc4C(F)(F)F)C[C@@H]3C)nc(=O)n(C([2H])([2H])[2H])c21]

A mixture of 2-(6-((2S,5R)-2,5-dimethylpiperazin-1-yl)-3,9-bis(methyl-d3)-2-oxo-3,9-dihydro-2H-purin-8-yl)acetonitrile (32.1 mg, 0.1 mmol), 1-(4-fluoro-2-(trifluoromethyl)phenyl)ethan-1-ol (41.6 mg, 0.2 mmol), (cyanomethyl)trimethylphosphonium iodide (73 mg, 0.3 mmol) and DIPEA (129 mg, 1 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to give the residue, which was purified by Pre-TLC (CH₂Cl₂/MeOH=10:1) to give the titled compound (15 mg). ¹H NMR (400 MHz, CD₃OD) δ 8.14-8.02 (m, 1H), 7.46-7.34 (m, 2H), 6.24-5.01 (m, 2H), 4.29-4.15 (m, 2H), 4.10-3.41 (m, 3H), 2.99-2.62 (m, 3H), 1.57-1.46 (m, 1.5H), 1.34-1.21 (m, 4.5H), 1.07-1.04 (m, 1.5H), 0.93-0.86 (m, 1.5H) ppm. MS: M/e 512 (M+1)⁺.

### Compound A37: 2-(6-((2S,5R)-4-(1-(4-fluoro-2-(trifluoromethyl)phenyl)ethyl)-2,5-dimethylpiperazin-1-yl)-3-methyl-9-(methyl-d3)-2-oxo-3,9-dihydro-2H-purin-8-yl)acetonitrile

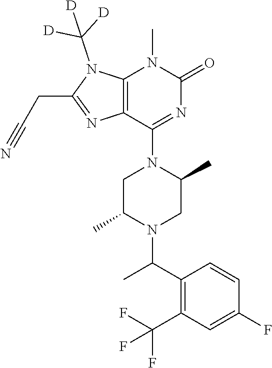 [SMILES: [2H]C([2H])([2H])n1c(CC#N)nc2c(N3C[C@@H](C)N(C(C)c4ccc(F)cc4C(F)(F)F)C[C@@H]3C)nc(=O)n(C)c21]

### Step A: tert-butyl (2R,5S)-2,5-dimethyl-4-(3-methyl-9-(methyl-d3)-2-oxo-3,9-dihydro-2H-purin-6-yl)piperazine-1-carboxylate

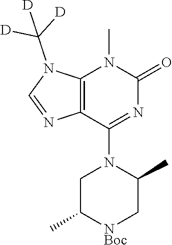 [SMILES: [2H]C([2H])([2H])n1cnc2c(N3C[C@@H](C)N(C(=O)OC(C)(C)C)C[C@@H]3C)nc(=O)n(C)c21]

To a stirred mixture of tert-butyl (2R,5S)-4-(2-hydroxy-9-(methyl-d3)-9H-purin-6-yl)-2,5-dimethylpiperazine-1-carboxylate (2 g, 5.46 mmol) in dioxane (50 mL) was added Cs₂CO₃ (5.3 g, 16.38 mmol), followed by Mel (1.54 g, 10.9 mmol). After then, the mixture was stirred overnight at 80° C. in a sealed tube. The mixture was poured into H₂O (150 mL), extracted with EtOAc (15 mL×3). The combined organic layers were washed with brine, dried over Na₂SO₄, concentrated to dryness. The resulting residue was purified by flash column chromatography to give the titled compound (860 mg, 41%). MS: M/e 380 (M+1)⁺.

### Step B: tert-butyl (2R,5S)-4-(8-formyl-3-methyl-9-(methyl-d3)-2-oxo-3,9-dihydro-2H-purin-6-yl)-2,5-dimethylpiperazine-1-carboxylate

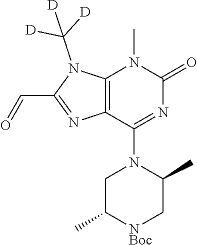 [SMILES: [2H]C([2H])([2H])n1c(C=O)nc2c(N3C[C@@H](C)N(C(=O)OC(C)(C)C)C[C@@H]3C)nc(=O)n(C)c21]

To a stirred solution of tert-butyl (2R,5S)-2,5-dimethyl-4-(3-methyl-9-(methyl-d3)-2-oxo-3,9-dihydro-2H-purin-6-yl)piperazine-1-carboxylate (860 mg, 2.25 mmol) in THE (15 mL) was added LDA (2.0 M, 4.5 mL, 9 mmol) dropwise at −78° C. After stirred for an hour, DMF (657 mg, 9 mmol) was added, and the mixture was stirred for an hour. The mixture was quenched with aq.NH₄Cl, extracted with EtOAc (20 mL×3). The combined organic layers were washed with brine, dried over Na₂SO₄, concentrated to dryness. The resulting residue was directly used to the next step without further purification. MS: M/e 408 (M+1)⁺.

### Step C: tert-butyl (2R,5S)-4-(8-(hydroxymethyl)-3-methyl-9-(methyl-d3)-2-oxo-3,9-dihydro-2H-purin-6-yl)-2,5-dimethylpiperazine-1-carboxylate

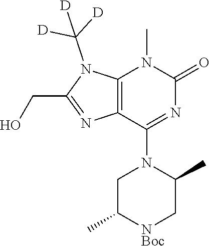 [SMILES: [2H]C([2H])([2H])n1c(CO)nc2c(N3C[C@@H](C)N(C(=O)OC(C)(C)C)C[C@@H]3C)nc(=O)n(C)c21]

To a stirred solution of tert-butyl (2R,5S)-4-(8-formyl-3-methyl-9-(methyl-d3)-2-oxo-3,9-dihydro-2H-purin-6-yl)-2,5-dimethylpiperazine-1-carboxylate (crude, 2.25 mmol) in MeOH (15 mL) was added NaBH₄ (128 mg, 3.37 mmol), then stirred for 10 min. The reaction mixture was poured into H₂O (30 mL), extracted with EtOAc (15 mL×3). The combined organic layers were washed with brine, dried over Na₂SO₄, concentrated and purified by flash column chromatography to give the titled compound (420 mg, 45%) as a white solid. MS: M/e 410 (M+1)⁺.

### Step D: tert-butyl (2R,5S)-4-(8-(chloromethyl)-3-methyl-9-(methyl-d3)-2-oxo-3,9-dihydro-2H-purin-6-yl)-2,5-dimethylpiperazine-1-carboxylate

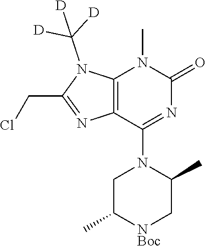 [SMILES: [2H]C([2H])([2H])n1c(CCl)nc2c(N3C[C@@H](C)N(C(=O)OC(C)(C)C)C[C@@H]3C)nc(=O)n(C)c21]

To a stirred solution of tert-butyl (2R,5S)-4-(8-(hydroxymethyl)-3-methyl-9-(methyl-d3)-2-oxo-3,9-dihydro-2H-purin-6-yl)-2,5-dimethylpiperazine-1-carboxylate (420 mg, 1.02 mmol) in CH₂Cl₂ (10 mL) was added SOCl₂ (243 mg, 2.04 mmol). After then, the reaction mixture was stirred for 10 min. The reaction mixture was concentrated to give the titled compound, which was directly used to the next step. MS: M/e 428 (M+1)⁺

### Step E: tert-butyl (2R,5S)-4-(8-(cyanomethyl)-3-methyl-9-(methyl-d3)-2-oxo-3,9-dihydro-2H-purin-6-yl)-2,5-dimethylpiperazine-1-carboxylate

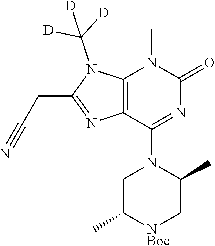 [SMILES: [2H]C([2H])([2H])n1c(CC#N)nc2c(N3C[C@@H](C)N(C(=O)OC(C)(C)C)C[C@@H]3C)nc(=O)n(C)c21]

To a stirred solution of tert-butyl (2R,5S)-4-(8-(chloromethyl)-3-methyl-9-(methyl-d3)-2-oxo-3,9-dihydro-2H-purin-6-yl)-2,5-dimethylpiperazine-1-carboxylate (crude, 1.02 mmol) in CH₂Cl₂ (15 mL) was added TBAF (1.0 M, 4 mL, 4.08 mmol), followed by TMSCN (408 mg, 4.08 mmol). After the addition, the reaction mixture was stirred for 4 hours. The reaction mixture was poured into H₂O (30 mL) and extracted with EtOAc (15 mL×3). The combined organic layers were washed with brine, dried over Na₂SO₄, concentrated to dryness. The resulting residue was purified by flash column chromatography to give the titled compound (240 mg, 56%). MS: M/e 419 (M+1)⁺

### Step F: 2-(6-((2S,5R)-2,5-dimethylpiperazin-1-yl)-3-methyl-9-(methyl-d3)-2-oxo-3,9-dihydro-2H-purin-8-yl)acetonitrile

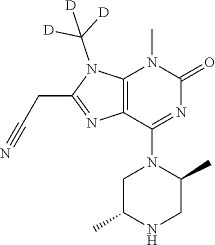 [SMILES: [2H]C([2H])([2H])n1c(CC#N)nc2c(N3C[C@@H](C)NC[C@@H]3C)nc(=O)n(C)c21]

To a stirred solution of tert-butyl (2R,5S)-4-(8-(cyanomethyl)-3-methyl-9-(methyl-d3)-2-oxo-3,9-dihydro-2H-purin-6-yl)-2,5-dimethylpiperazine-1-carboxylate (240 mg, 0.57 mmol) in CH₂Cl₂ (10 mL) was added TFA (2 mL). After the addition, the reaction mixture was stirred for an hour. The reaction mixture was concentrated to give the residue, which was basified to pH=10˜12 with aq.Na₂CO₃ and extracted with CH₂Cl₂/IPA (3/1, 30 mL×4). The combined organic layers were washed with brine, dried over Na₂SO₄, concentrated to give the titled compound (120 mg, 66%). MS: M/e 319 (M+1)⁺

### Step G: 2-(6-((2S,5R)-4-(1-(4-fluoro-2-(trifluoromethyl)phenyl)ethyl)-2,5-dimethylpiperazin-1-yl)-3-methyl-9-(methyl-d3)-2-oxo-3,9-dihydro-2H-purin-8-yl)acetonitrile

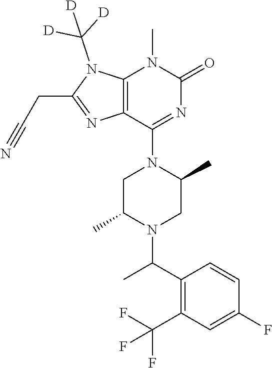 [SMILES: [2H]C([2H])([2H])n1c(CC#N)nc2c(N3C[C@@H](C)N(C(C)c4ccc(F)cc4C(F)(F)F)C[C@@H]3C)nc(=O)n(C)c21]

A mixture of 2-(6-((2S,5R)-2,5-dimethylpiperazin-1-yl)-3-methyl-9-(methyl-d3)-2-oxo-3,9-dihydro-2H-purin-8-yl)acetonitrile (31.8 mg, 0.1 mmol), 1-(4-fluoro-2-(trifluoromethyl)phenyl)ethan-1-ol (41.6 mg, 0.2 mmol), (cyanomethyl)trimethylphosphonium iodide (73 mg, 0.3 mmol) and DIPEA (129 mg, 1 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to give the residue, which was purified by Pre-TLC (EtOAc, then CH₂Cl₂/MeOH=10:1) to give the titled compound (5 mg). ¹H NMR (400 MHz, CD₃OD) δ 8.12-8.03 (m, 1H), 7.46-7.36 (m, 2H), 6.23-5.00 (m, 2H), 4.25-4.15 (m, 2H), 4.10-3.85 (m, 1H), 3.78 (s, 3H), 3.68-3.41 (m, 1H), 3.00-2.63 (m, 2.5H), 1.99 (d, J=12.4 Hz, 0.5H), 1.56-1.19 (m, 6H), 1.07-0.88 (m, 3H) ppm. MS: M/e 509 (M+1)⁺.

### Compound A41: 2-(6-((2S,5R)-2,5-dimethyl-4-(1-(3-(trifluoromethyl)phenyl)ethyl)piperazin-1-yl)-3,9-dimethyl-2-oxo-3,9-dihydro-2H-purin-8-yl)acetonitrile

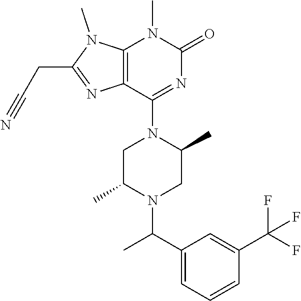 [SMILES: CC(c1cccc(C(F)(F)F)c1)N1C[C@H](C)N(c2nc(=O)n(C)c3c2nc(CC#N)n3C)C[C@H]1C]

### Step A: 2-(6-((2S,5R)-2,5-dimethyl-4-(1-(3-(trifluoromethyl)phenyl)ethyl)piperazin-1-yl)-3,9-dimethyl-2-oxo-3,9-dihydro-2H-purin-8-yl)acetonitrile

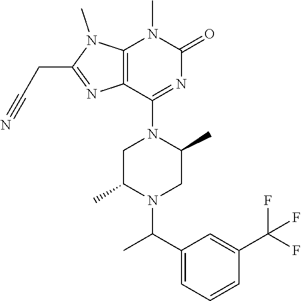 [SMILES: CC(c1cccc(C(F)(F)F)c1)N1C[C@H](C)N(c2nc(=O)n(C)c3c2nc(CC#N)n3C)C[C@H]1C]

To a solution of 2-(6-((2S,5R)-2,5-dimethylpiperazin-1-yl)-3,9-dimethyl-2-oxo-3,9-dihydro-2H-purin-8-yl)acetonitrile (100 mg, 0.31 mmol) in CH₃CN (2 mL) and was added 1-(3-(trifluoromethyl)phenyl)ethan-1-ol (180 mg, 0.95 mmol), (cyanomethyl)trimethylphosphonium iodide (308 mg, 1.27 mmol) and DIPEA (0.4 g, 3.1 mmol). The resulting mixture was stirred at 105° C. overnight. The reaction solvent was removed under reduced pressure and purified by Prep-TLC (DCM/MeOH=30/1) to give the titled compound (crude), which was further purified to give the titled compound (20 mg) by Prep-HPLC (Method A). ¹H NMR (400 MHz, CD₃OD) δ 7.78-7.46 (m, 4H), 6.01-4.95 (m, 2H), 4.21 (s, 2H), 3.93 (s, 3H), 3.78 (s, 3H), 3.74-3.45 (m, 2H), 3.05-2.75 (m, 2H), 2.73-2.03 (m, 1H), 1.98-1.21 (m, 6H), 1.15-0.83 (m, 3H) ppm. MS: M/e 488 (M+1)⁺.

### Compound A42: 2-(6-((2S,5R)-2,5-dimethyl-4-(1-(3-(trifluoromethyl)phenyl)ethyl)piperazin-1-yl)-9-ethyl-3-methyl-2-oxo-3,9-dihydro-2H-purin-8-yl)acetonitrile

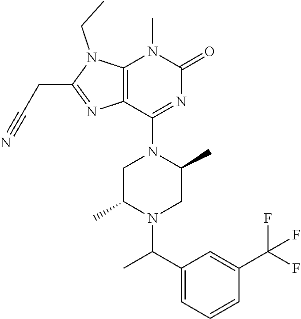 [SMILES: CCn1c(CC#N)nc2c(N3C[C@@H](C)N(C(C)c4cccc(C(F)(F)F)c4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (300 mg, 0.9 mmol) in CH₃CN (8 mL) and was added 1-(3-(trifluoromethyl)phenyl)ethan-1-ol (520 mg, 2.7 mmol), (cyanomethyl)trimethylphosphonium iodide (875 mg, 3.6 mmol) and DIPEA (1.16 g, 9 mmol). The resulting mixture was stirred at 105° C. overnight. The reaction solvent was removed under reduced pressure and purified by Prep-TLC (DCM/MeOH=30/1) to give the titled Compound A42, which was further separated into Compound A42a (68.23 mg) and Compound A42b (76.15 mg) by chiral Prep-HPLC. The chiral separation conditions are shown below.

| | |
|---|---|
| Column | CHIRALPAK IH |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A42: ¹H NMR (400 MHz, CD₃OD) δ 7.78-7.60 (m, 2H), 7.60-7.46 (m, 2H), 6.25-5.52 (m, 1H), 5.43-5.01 (m, 0.5H), 4.73-4.62 (m, 0.5H), 4.37 (q, J=7.0 Hz, 2H), 4.23 (s, 2H), 3.85-3.72 (m, 4H), 3.67-3.40 (m, 1.5H), 2.94-2.54 (m, 2H), 2.13 (d, J=12.2 Hz, 0.5H), 1.46 (t, J=6.9 Hz, 4.5H), 1.39-1.31 (m, 3H), 1.26 (d, J=5.2 Hz, 1.5H), 1.06 (d, J=6.3 Hz, 1.5H), 0.92 (d, J=6.4 Hz, 1.5H) ppm. MS: M/e 502 (M+1)⁺

Compound A42a (the earlier peak): ¹H NMR (400 MHz, CD₃OD) δ 7.73-7.63 (m, 2H), 7.60-7.49 (m, 2H), 6.35-4.51 (m, 2H), 4.37 (d, J=7.1 Hz, 2H), 4.22 (s, 2H), 3.76 (s, 3H), 3.73 (s, 1H), 3.65-3.36 (m, 1H), 2.85 (d, J=20.8 Hz, 3H), 1.52-1.40 (m, 6H), 1.33 (d, J=6.5 Hz, 3H), 0.91 (d, J=6.4 Hz, 3H) ppm. MS: M/e 502 (M+1)⁺.

Compound A42b (the later peak): ¹H NMR (400 MHz, CD₃OD) δ 7.75 (s, 1H), 7.63 (d, J=7.1 Hz, 1H), 7.60-7.47 (m, 2H), 6.05-4.95 (m, 2H), 4.37 (q, J=7.2 Hz, 2H), 4.23 (s, 2H), 3.76 (s, 3H), 3.56 (dd, J=30.0, 23.5 Hz, 3H), 2.68 (s, 1H), 2.13 (d, J=12.1 Hz, 1H), 1.46 (t, J=7.2 Hz, 3H), 1.35 (d, J=6.5 Hz, 3H), 1.27 (s, 3H), 1.06 (d, J=6.3 Hz, 3H) ppm. MS: M/e 502 (M+1)⁺.

### Compound A43: 2-(9-ethyl-6-((2S,5R)-4-(1-(3-isopropoxyphenyl)ethyl)-2,5-dimethylpiperazin-1-yl)-3-methyl-2-oxo-3,9-dihydro-2H-purin-8-yl)acetonitrile

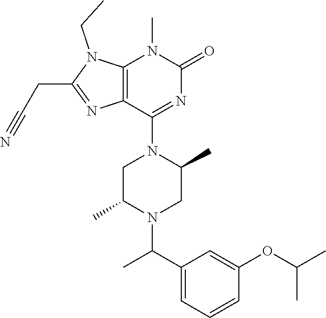 [SMILES: CCn1c(CC#N)nc2c(N3C[C@@H](C)N(C(C)c4cccc(OC(C)C)c4)C[C@@H]3C)nc(=O)n(C)c21]

### Step A: 1-(3-isopropoxyphenyl)ethan-1-ol

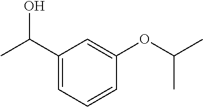

To a solution of 3-isopropoxybenzaldehyde (500 mg, 3.045 mmol) in THE (30 mL) was degassed 3 times under N₂ atmosphere. Then added CH₃MgBr (2 mL, 3M, 6.09 mmol) dropwise at 0° C. The mixture was stirred at 0° C. for 4 hours and diluted with saturated NH₄Cl, extracted with EtOAc (150 mL×2), washed with brine, dried over Na₂SO₄, filtered, and concentrated to dryness. The resulting residue was purified by flash column chromatography (PE:EtOAc=4:1) to give the titled compound (500 mg, 91%). ¹H NMR (400 MHz, CDCl₃) δ 7.29-7.21 (m, 1H), 6.92 (d, J=6.7 Hz, 2H), 6.85-6.73 (m, 1H), 4.86 (q, J=6.4 Hz, 1H), 4.57 (dt, J=12.1, 6.0 Hz, 1H), 1.79 (s, 1H), 1.48 (d, J=6.4 Hz, 3H), 1.34 (d, J=6.1 Hz, 6H) ppm.

### Step B: 2-(9-ethyl-6-((2S,5R)-4-(1-(3-isopropoxyphenyl)ethyl)-2,5-dimethylpiperazin-1-yl)-3-methyl-2-oxo-3,9-dihydro-2H-purin-8-yl)acetonitrile

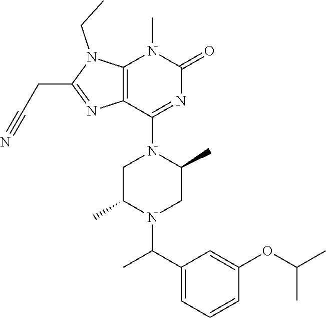 [SMILES: CCn1c(CC#N)nc2c(N3C[C@@H](C)N(C(C)c4cccc(OC(C)C)c4)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (120 mg, 0.365 mmol), 1-(3-isopropoxyphenyl)ethan-1-ol (197 mg, 1.094 mmol), (cyanomethyl)trimethylphosphonium iodide (177 mg. 0.730 mmol) and DIPEA (141 mg, 1.094 mol) in CH₃CN (2 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) and Prep-HPLC (Method A) to give the titled compound (58 mg, 32%). ¹H NMR (400 MHz, CD₃OD) δ 7.26-7.14 (m, 1H), 6.93 (dd, J=19.2, 6.1 Hz, 2H), 6.79 (t, J=9.2 Hz, 1H), 6.23-5.53 (m, 1H), 5.26-5.01 (m, 0.5H), 4.85-4.71 (m, 0.5H), 4.59 (dd, J=11.9, 6.0 Hz, 1H), 4.37 (q, J=6.9 Hz, 2H), 4.22 (s, 1H), 3.76 (s, 3H), 3.61 (s, 1H), 3.48 (s, 2H), 3.10-2.58 (m, 2.5H), 2.29 (d, J=11.7 Hz, 0.5H), 1.46 (t, J=6.9 Hz, 5H), 1.31 (t, J=6.1 Hz, 10H), 0.98 (dd, J=55.1, 6.2 Hz, 3H) ppm. MS: M/e 492 (M+1)⁺

### Compound A44: 2-(6-((2S,5R)-2,5-dimethyl-4-(1-(3-(trifluoromethoxy)phenyl)ethyl)piperazin-1-yl)-9-ethyl-3-methyl-2-oxo-3,9-dihydro-2H-purin-8-yl)acetonitrile

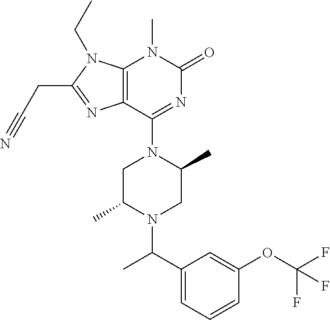 [SMILES: CCn1c(CC#N)nc2c(N3C[C@@H](C)N(C(C)c4cccc(OC(F)(F)F)c4)C[C@@H]3C)nc(=O)n(C)c21]

To a solution of 1-(3-(trifluoromethoxy)phenyl)ethan-1-ol (60 mg, 0.29 mmol), 2-(6-((2S,5R)-2,5-dimethylpiperazin-1-yl)-9-ethyl-3-methyl-2-oxo-3,9-dihydro-2H-purin-8-yl)acetonitrile (60 mg, 0.19 mmol) and (cyanomethyl) trimethyl phosphonium iodide (68 mg, 0.28 mmol) in CH₃CN (5 mL) was added DIPEA (116 mg, 0.9 mmol). The mixture was sealed in a bottle and heated at 100° C. for 16 hours. The mixture was cooled to room temperature, diluted with water, extracted with EtOAc (10 mL×2), washed with brine, dried over Na₂SO₄, filtered, and concentrated to dryness. The resulting residue was purified by Prep-TLC (DCM:MeOH=15:1) to give the titled compound (19 mg, 19%). ¹H NMR (400 MHz, CD₃OD) δ 7.45-7.34 (m, 3H), 7.17-7.12 (m, 1H), 5.97-5.91 (m, 1H), 5.31-5.24 (m, 1H), 4.38-4.34 (m, 2H), 4.22 (s, 2H), 3.76 (s, 3H), 3.59-3.48 (m, 2H), 2.88-2.84 (m, 2H), 2.70-2.66 (m, 0.5H), 2.18-2.15 (m, 0.5H), 1.49-1.44 (m, 4H), 1.35-1.29 (m, 4H), 1.06-1.02 (m, 2H), 0.92-0.90 (m, 2H) ppm. MS: M/e 518 (M+1)⁺.

### Compound A48: 2-(6-((2S,5R)-2,5-dimethyl-4-(1-(quinoxalin-6-yl)ethyl)piperazin-1-yl)-3,9-dimethyl-2-oxo-3,9-dihydro-2H-purin-8-yl)acetonitrile

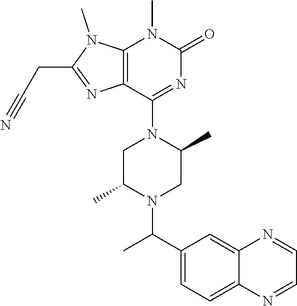 [SMILES: CC(c1ccc2nccnc2c1)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl) acetonitrile (50 mg, 0.117 mol), 1-(quinoxalin-6-yl)ethan-1-ol (41 mg, 0.233 mol), (cyanomethyl)trimethylphosphonium iodide (57 mg. 0.233 mol) and DIPEA (45 mg, 0.351 mol) in CH₃CN (2 ml). The mixture solution was degassed 3 times under N₂ atmosphere. Then the mixture solution was stirred at 105° C. for 24 hours. The reaction mixture was concentrated under reduced pressure. The resulting residue was purified by Prep-HPLC (Method A) to give the titled compound (7 mg, 13%). ¹H NMR (400 MHz, CD₃OD) 8.87 (t, J=3.3 Hz, 2H), 8.13-8.02 (m, 3H), 5.90-4.65 (m, 2H), 4.26-4.15 (m, 2H), 4.00-3.96 (m, 1H), 3.94 (s, 3H), 3.78 (s, 3H), 3.68-3.49 (m, 1H), 3.15-2.00 (m, 3H), 1.54-1.49 (m, 1.5H), 1.44 (dd, J=12.1, 6.5 Hz, 3H), 1.31-1.27 (m, 1.5H), 1.11 (d, J=6.5 Hz, 1.5H), 0.96 (d, J=6.3 Hz, 1.5H) ppm. MS: M/e 472 (M+1)⁺.

### Compound A49: 2-(6-((2S,5R)-2,5-dimethyl-4-(1-(3-methylquinoxalin-6-yl)ethyl)piperazin-1-yl)-3,9-dimethyl-2-oxo-3,9-dihydro-2H-purin-8-yl)acetonitrile

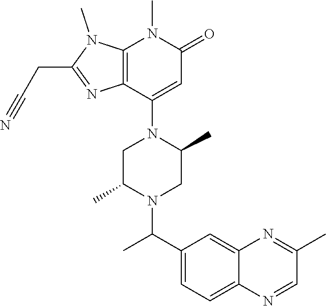 [SMILES: Cc1cnc2ccc(C(C)N3C[C@H](C)N(c4cc(=O)n(C)c5c4nc(CC#N)n5C)C[C@H]3C)cc2n1]

A solution of 2-(6-((2S,5R)-2,5-dimethylpiperazin-1-yl)-3,9-dimethyl-2-oxo-3,9-dihydro-2H-purin-8-yl)acetonitrile (315 mg, 1 mol), 1-(3-methylquinoxalin-6-yl)ethan-1-ol (376 mg, 2 mol), (cyanomethyl)trimethylphosphonium iodide (486 mg. 2 mol) and DIPEA (387 mg, 3 mol) in CH₃CN (3 ml). The mixture solution was degassed 3 times under N₂ atmosphere. Then the mixture solution was stirred at 105° C. for 24 hours. The reaction mixture was concentrated under reduced pressure. The crude product was purified by Prep-TLC (DCM:MeOH=15:1) to give the titled Compound A49 (80 mg), which was further separated into Compound A49a (32 mg) and Compound A49b (35 mg) by Prep-HPLC (Method A).

Compound A49: ¹H NMR (400 MHz, CD₃OD) δ 8.79 (d, J=4.7 Hz, 1H), 8.09-7.90 (m, 3H), 6.27-4.55 (m, 2H), 4.20 (s, 2H), 3.96-3.86 (m, 4H), 3.78 (s, 3H), 3.76-3.60 (m, 2H), 2.97-2.87 (m, 1.5H), 2.77 (d, J=1.6 Hz, 3H), 2.20 (d, J=11.5 Hz, 0.5H), 1.45-1.39 (m, 3H), 1.36-1.22 (m, 3H), 1.10-0.94 (m, 3H) ppm. MS: M/e 486 (M+1)⁺.

Compound A49a (the earlier peak): ¹H NMR (400 MHz, CD₃OD) δ 8.79 (s, 1H), 8.06 (d, J=8.6 Hz, 1H), 7.98 (s, 1H), 7.93 (dd, J=8.7, 1.6 Hz, 1H), 6.29-5.16 (m, 2H), 4.58 (s, 1H), 4.30-4.16 (m, 2H), 3.96-3.89 (m, 4H), 3.79 (s, 3H), 3.00-2.85 (m, 3H), 2.77 (s, 3H), 1.50 (s, 3H), 1.41 (t, J=8.9 Hz, 3H), 0.94 (t, J=8.4 Hz, 3H) ppm. MS: M/e 486 (M+1)⁺.

Compound A49b (the later peak): ¹H NMR (400 MHz, CD₃OD) δ 8.78 (s, 1H), 8.03 (d, J=8.6 Hz, 1H), 7.96 (s, 1H), 7.92 (dd, J=8.7, 1.6 Hz, 1H), 5.96-5.68 (m, 1H), 5.08-4.92 (m, 1H), 4.58 (s, 1H), 4.20 (s, 2H), 3.94 (s, 3H), 3.80-3.73 (m, 4H), 3.67-3.61 (m, 1H), 2.79-2.73 (m, 4H), 2.20 (d, J=11.5 Hz, 1H), 1.45 (d, J=6.5 Hz, 3H), 1.28 (d, J=5.6 Hz, 3H), 1.10 (d, J=6.5 Hz, 3H) ppm. MS: M/e 486 (M+1)⁺.

### Compound A50: 2-(6-((2S,5R)-5-ethyl-2-methyl-4-(1-(3-methylquinoxalin-6-yl)ethyl)piperazin-1-yl)-3,9-dimethyl-2-oxo-3,9-dihydro-2H-purin-8-yl)acetonitrile

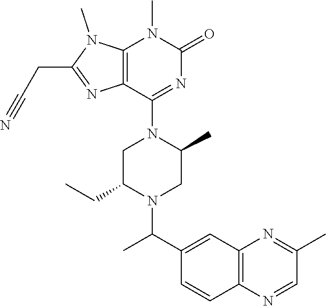 [SMILES: CC[C@@H]1CN(c2nc(=O)n(C)c3c2nc(CC#N)n3C)[C@@H](C)CN1C(C)c1ccc2ncc(C)nc2c1]

### Step A: tert-butyl (2S,5R)-5-ethyl-2-methyl-4-(1-(3-methylquinoxalin-6-yl)ethyl)piperazine-1-carboxylate

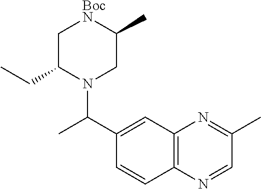 [SMILES: CC[C@@H]1CN(C(=O)OC(C)(C)C)[C@@H](C)CN1C(C)c1ccc2ncc(C)nc2c1]

A solution of 1-(3-methylquinoxalin-6-yl)ethan-1-ol (500 mg, 2.66 mmol), tert-butyl (2S,5R)-5-ethyl-2-methylpiperazine-1-carboxylate (758 mg, 3.32 mmol), (cyanomethyl)trimethylphosphonium iodide (1.29 g, 5.31 mmol) and DIPEA (2.74 g, 21.24 mmol) in MeCN (6 ml) was stirred at 100° C. overnight. The reaction was diluted with EtOAc (20 ml) and washed with brine (10 ml). The organic layer was concentrated to dryness. The resulting residue was purified by flash column chromatography with 0-20% EtOAc in PE to give the titled compound product (1 g, 94%). MS: M/e 399 (M+1)⁺.

### Step B: 7-(1-((2R,5S)-2-ethyl-5-methylpiperazin-1-yl)ethyl)-2-methylquinoxaline

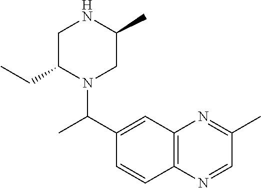 [SMILES: CC[C@@H]1CN[C@@H](C)CN1C(C)c1ccc2ncc(C)nc2c1]

A solution of tert-butyl (2S,5R)-5-ethyl-2-methyl-4-(1-(3-methylquinoxalin-6-yl)ethyl)piperazine-1-carboxylate (1 g, 2.51 mmol) and TFA (4 ml) in DCM (16 ml) was stirred at rt for 60 min. The reaction was concentrated under reduced pressure. The residue was diluted with EtOAc (20 ml) and washed with aq. NaHCO₃ (10 ml×2). The organic layer was dried over Na₂SO₄ and concentrated to dryness. The resulting residue was purified by flash column chromatography with 0-5% MeOH in DCM to give the titled compound product (745 mg, 100%). MS: M/e 299 (M+1)⁺.

### Step C: 8-((benzyloxy)methyl)-6-((2S,5R)-5-ethyl-2-methyl-4-(1-(3-methylquinoxalin-6-yl)ethyl)piperazin-1-yl)-3,9-dimethyl-3,9-dihydro-2H-purin-2-one

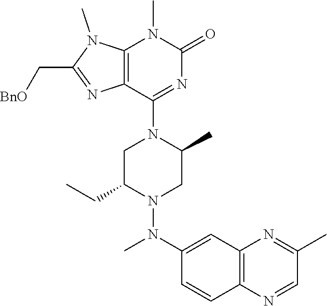 [SMILES: CC[C@@H]1CN(c2nc(=O)n(C)c3c2nc(COCc2ccccc2)n3C)[C@@H](C)CN1N(C)c1ccc2ncc(C)nc2c1]

A solution of 7-(1-((2R,5S)-2-ethyl-5-methylpiperazin-1-yl)ethyl)-2-methylquinoxaline (645 mg, 2.16 mmol), 8-((benzyloxy)methyl)-6-hydroxy-3,9-dimethyl-3,9-dihydro-2H-purin-2-one (649 mg, 2.16 mmol), BOP (1.44 g, 3.25 mmol) and DBU (993 mg, 6.49 mmol) in CH₃CN (15 ml) was stirred at rt for 24 h. The reaction was concentrated to dryness. The resulting residue was diluted with EtOAc (20 ml) and washed with brine (10 ml). The organic layer was dried over Na₂SO₄ and concentrated. The residue was purified by flash column chromatography with 0-10% MeOH in DCM to give the titled compound (crude), which was used directly for the next step without further purification. MS: M/e 581 (M+1)⁺.

### Step D: 6-((2S,5R)-5-ethyl-2-methyl-4-(1-(3-methylquinoxalin-6-yl)ethyl)piperazin-1-yl)-8-(hydroxymethyl)-3,9-dimethyl-3,9-dihydro-2H-purin-2-one

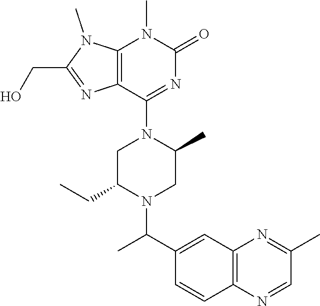 [SMILES: CC[C@@H]1CN(c2nc(=O)n(C)c3c2nc(CO)n3C)[C@@H](C)CN1C(C)c1ccc2ncc(C)nc2c1]

A solution of 8-((benzyloxy)methyl)-6-((2S,5R)-5-ethyl-2-methyl-4-(1-(3-methylquinoxalin-6-yl)ethyl)piperazin-1-yl)-3,9-dimethyl-3,9-dihydro-2H-purin-2-one (crude) in TFA (20 ml) was stirred at 66° C. for 2 days. The solution was concentrated to dryness. The residue was diluted with EtOAc (20 ml), washed with aq. NaHCO₃ (10 ml×2) and brine (10 ml), dried over Na₂SO₄ and concentrated. The resulting residue was purified by flash column chromatography with 0-15% MeOH in DCM to give the titled compound (580 mg) as an off-white solid. MS: M/e 491 (M+1)⁺.

### Step E: 8-(chloromethyl)-6-((2S,5R)-5-ethyl-2-methyl-4-(1-(3-methylquinoxalin-6-yl)ethyl)piperazin-1-yl)-3,9-dimethyl-3,9-dihydro-2H-purin-2-one

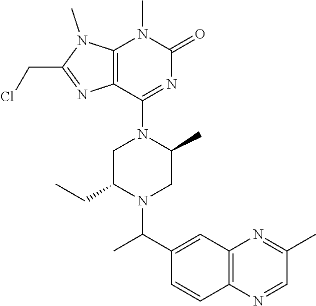 [SMILES: CC[C@@H]1CN(c2nc(=O)n(C)c3c2nc(CCl)n3C)[C@@H](C)CN1C(C)c1ccc2ncc(C)nc2c1]

To a solution of 6-((2S,5R)-5-ethyl-2-methyl-4-(1-(3-methylquinoxalin-6-yl)ethyl)piperazin-1-yl)-8-(hydroxymethyl)-3,9-dimethyl-3,9-dihydro-2H-purin-2-one (580 mg, 1.18 mmol) in DCM (15 ml) at 0° C., was added SOCl₂ (423 mg, 3.55 mmol) and stirred at 0° C. for 20 min. The reaction was washed with aq. NaHCO₃ (10 ml) and brine (10 ml), dried over Na₂SO₄ and concentrated to dryness to give the titled compound (601 mg, 100%), which was used directly for the next step. MS: M/e 509 (M+1)⁺.

### Step F: 2-(6-((2S,5R)-5-ethyl-2-methyl-4-(1-(3-methylquinoxalin-6-yl)ethyl)piperazin-1-yl)-3,9-dimethyl-2-oxo-3,9-dihydro-2H-purin-8-yl)acetonitrile

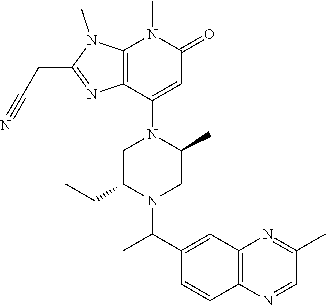 [SMILES: CC[C@@H]1CN(c2cc(=O)n(C)c3c2nc(CC#N)n3C)[C@@H](C)CN1C(C)c1ccc2ncc(C)nc2c1]

A solution of 8-(chloromethyl)-6-((2S,5R)-5-ethyl-2-methyl-4-(1-(3-methylquinoxalin-6-yl)ethyl)piperazin-1-yl)-3,9-dimethyl-3,9-dihydro-2H-purin-2-one (601 mg, 1.18 mmol), TMSCN (351 mg, 3.55 mmol) and Cs₂CO₃ (1.15 g, 3.53 mmol) in MeCN (15 ml) was stirred at 66° C. for 60 min. The reaction mixture was poured into water (15 ml) and then extracted with EtOAc (20 ml×2). The organic layer was washed with brine (10 ml), dried over Na₂SO₄ and concentrated to dryness. The resulting residue was purified by flash column chromatography with 0-10% MeOH in DCM to give the titled Compound A50, which was separated into Compound A50a (40 mg) and Compound A50b (60 mg) by chiral Prep-HPLC. The chiral separation conditions are shown below.

| | |
|---|---|
| Column | CHIRALPAK IA |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A50a (the earlier peak): ¹H NMR (400 MHz, DMSO-d6) δ 8.83 (s, 1H), 8.05 (d, J=8.6 Hz, 1H), 7.92 (s, 1H), 7.85 (d, J=8.6 Hz, 1H), 5.97 (s, 0.5H), 5.70 (d, J=14.0 Hz, 0.5H), 5.05 (s, 0.5H), 4.64 (d, J=14.0 Hz, 0.5H), 4.36 (d, J=33.8 Hz, 2H), 3.98 (q, J=6.5 Hz, 1H), 3.82 (d, J=23.8 Hz, 3H), 3.64 (s, 3H), 3.31 (s, 0.5H), 3.00 (s, 0.5H), 2.82 (d, J=25.2 Hz, 2H), 2.70 (s, 3H), 2.34 (s, 1H), 1.56-1.47 (m, 1H), 1.45-1.39 (m, 1H), 1.38-1.30 (m, 6H), 0.63 (t, J=7.2 Hz, 3H) ppm. MS: M/e 500 (M+1)⁺.

Compound A50b (the later peak): ¹H NMR (400 MHz, DMSO-d6) δ 8.81 (s, 1H), 8.02 (d, J=8.7 Hz, 1H), 7.95 (s, 1H), 7.86 (d, J=8.6 Hz, 1H), 5.97 (d, J=13.2 Hz, 0.5H), 5.73 (s, 0.5H), 4.98-4.80 (m, 1H), 4.37 (d, J=37.3 Hz, 2H), 3.83 (s, 4H), 3.64 (s, 3H), 3.59 (s, 0.5H), 3.32-3.19 (m, 0.5H), 3.11 (s, 1H), 2.70 (s, 3H), 2.55 (d, J=9.0 Hz, 1H), 2.16 (d, J=11.8 Hz, 1H), 1.51-1.41 (m, 2H), 1.34 (d, J=6.2 Hz, 3H), 1.21-1.10 (m, 3H), 0.99 (t, J=9.2 Hz, 3H) ppm. MS: M/e 500 (M+1)⁺.

### Compound A51: 2-(6-((2S,5R)-2,5-diethyl-4-(1-(3-methylquinoxalin-6-yl)ethyl)piperazin-1-yl)-3,9-dimethyl-2-oxo-3,9-dihydro-2H-purin-8-yl)acetonitrile

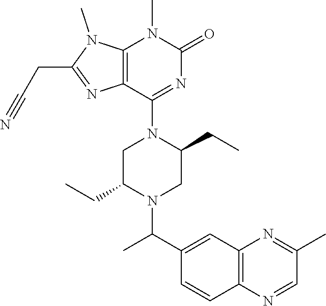 [SMILES: CC[C@H]1CN(C(C)c2ccc3ncc(C)nc3c2)[C@H](CC)CN1c1nc(=O)n(C)c2c1nc(CC#N)n2C]

### Step A: tert-butyl (2S,5R)-2,5-diethyl-4-(1-(3-methylquinoxalin-6-yl)ethyl)piperazine-1-carboxylate

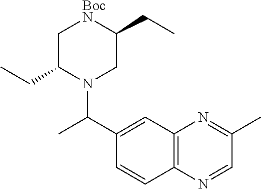 [SMILES: CC[C@H]1CN(C(C)c2ccc3ncc(C)nc3c2)[C@H](CC)CN1C(=O)OC(C)(C)C]

A solution of 1-(3-methylquinoxalin-6-yl)ethan-1-ol (500 mg, 2.66 mmol), tert-butyl (2S,5R)-2,5-diethylpiperazine-1-carboxylate (805 mg, 3.33 mmol), (cyanomethyl)trimethylphosphonium iodide (1.29 g, 5.31 mmol) and DIPEA (2.74 g, 21.24 mmol) in MeCN (6 ml) was stirred at 100° C. overnight. The reaction was diluted with EtOAc (20 ml) and washed with brine (10 ml). The organic layer was concentrated to dryness. The resulting residue was purified by flash column chromatography with 0-20% EtOAc in PE to give the titled compound (1 g, 91%). MS: M/e 413 (M+1)⁺.

### Step B: 7-(1-((2R,5S)-2,5-diethylpiperazin-1-yl)ethyl)-2-methylquinoxaline

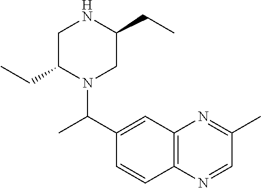 [SMILES: CC[C@H]1CN(C(C)c2ccc3ncc(C)nc3c2)[C@H](CC)CN1]

A solution of tert-butyl (2S,5R)-2,5-diethyl-4-(1-(3-methylquinoxalin-6-yl)ethyl)piperazine-1-carboxylate (1 g, 2.43 mmol) and TFA (4 ml) in DCM (16 ml) was stirred at rt for 30 min. The reaction was concentrated under reduced pressure. The residue was diluted with EtOAc (20 ml) and washed with aq. NaHCO₃ (10 ml×2). The organic layer was dried over Na₂SO₄ and concentrated to dryness. The resulting residue was purified by flash column chromatography with 0-5% MeOH in DCM to give the titled compound (757 mg, 100%). MS: M/e 313 (M+1)⁺.

### Step C: 8-((benzyloxy)methyl)-6-((2S,5R)-2,5-diethyl-4-(1-(3-methylquinoxalin-6-yl)ethyl)piperazin-1-yl)-3,9-dimethyl-3,9-dihydro-2H-purin-2-one

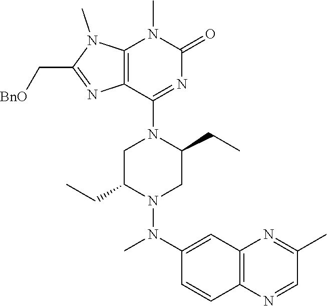 [SMILES: CC[C@H]1CN(N(C)c2ccc3ncc(C)nc3c2)[C@H](CC)CN1c1nc(=O)n(C)c2c1nc(COCc1ccccc1)n2C]

A solution of 7-(1-((2R,5S)-2,5-diethylpiperazin-1-yl)ethyl)-2-methylquinoxaline (400 mg, 1.28 mmol), 8-((benzyloxy)methyl)-6-hydroxy-3,9-dimethyl-3,9-dihydro-2H-purin-2-one (423 mg, 1.41 mmol), BOP (737 mg, 1.67 mmol) and DBU (392 mg, 2.56 mmol) in MeCN (12 ml) was stirred at rt for 24 h and then 50° C. overnight. The reaction was concentrated to dryness. The resulting residue was diluted with EtOAc (20 ml) and washed with brine (10 ml). The organic layer was dried over Na₂SO₄ and concentrated. The residue was purified by flash column chromatography with 0-10% MeOH in DCM to give the titled compound (crude), which was used directly for the next step without further purification. MS: M/e 595 (M+1)⁺.

### Step D: 6-((2S,5R)-2,5-diethyl-4-(1-(3-methylquinoxalin-6-yl)ethyl)piperazin-1-yl)-8-(hydroxymethyl)-3,9-dimethyl-3,9-dihydro-2H-purin-2-one

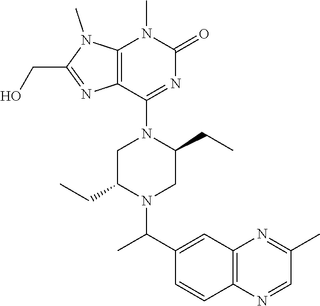 [SMILES: CC[C@H]1CN(C(C)c2ccc3ncc(C)nc3c2)[C@H](CC)CN1c1nc(=O)n(C)c2c1nc(CO)n2C]

A solution of 8-((benzyloxy)methyl)-6-((2S,5R)-2,5-diethyl-4-(1-(3-methylquinoxalin-6-yl)ethyl)piperazin-1-yl)-3,9-dimethyl-3,9-dihydro-2H-purin-2-one (crude) in TFA (10 ml) was stirred at 66° C. for 3 days. The solution was concentrated to dryness. The residue was diluted with EtOAc (20 ml), washed with aq. NaHCO₃ (10 ml×2) and brine (10 ml), dried over Na₂SO₄ and concentrated. The resulting residue was purified by flash column chromatography with 0-15% MeOH in EtOAc to give the titled compound (250 mg). MS: M/e 505 (M+1)⁺.

### Step E: 8-(chloromethyl)-6-((2S,5R)-2,5-diethyl-4-(1-(3-methylquinoxalin-6-yl)ethyl)piperazin-1-yl)-3,9-dimethyl-3,9-dihydro-2H-purin-2-one

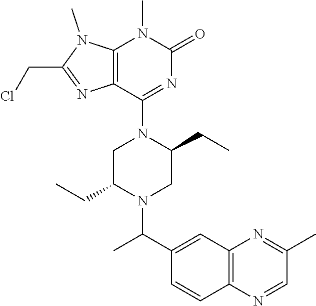 [SMILES: CC[C@H]1CN(C(C)c2ccc3ncc(C)nc3c2)[C@H](CC)CN1c1nc(=O)n(C)c2c1nc(CCl)n2C]

To a solution of 6-((2S,5R)-2,5-diethyl-4-(1-(3-methylquinoxalin-6-yl)ethyl)piperazin-1-yl)-8-(hydroxymethyl)-3,9-dimethyl-3,9-dihydro-2H-purin-2-one (250 mg, 0.50 mmol) in DCM (15 ml) at 0° C., was added SOCl₂ (177 mg, 1.49 mmol) and stirred at 0° C. for 25 min. The reaction was washed with aq. NaHCO₃ (10 ml) and brine (10 ml), dried over Na₂SO₄ and concentrated to dryness to give the titled compound (259 mg, 100%), which was used directly for the next step. MS: M/e 523 (M+1)⁺.

### Step F: 2-(6-((2S,5R)-2,5-diethyl-4-(1-(3-methylquinoxalin-6-yl)ethyl)piperazin-1-yl)-3,9-dimethyl-2-oxo-3,9-dihydro-2H-purin-8-yl)acetonitrile

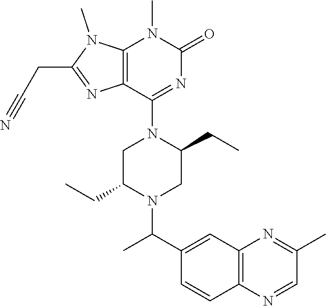 [SMILES: CC[C@H]1CN(C(C)c2ccc3ncc(C)nc3c2)[C@H](CC)CN1c1nc(=O)n(C)c2c1nc(CC#N)n2C]

A solution of 8-(chloromethyl)-6-((2S,5R)-2,5-diethyl-4-(1-(3-methylquinoxalin-6-yl)ethyl)piperazin-1-yl)-3,9-dimethyl-3,9-dihydro-2H-purin-2-one (259 mg, 0.50 mmol), TMSCN (148 mg, 1.49 mmol) and Cs₂CO₃ (485 mg, 1.49 mmol) in MeCN (10 ml) was stirred at 70° C. for 60 min. The reaction mixture was poured into water (15 ml) and then extracted with EtOAc (15 ml×2). The organic layer was washed with brine (10 ml), dried over Na₂SO₄ and concentrated to dryness. The resulting residue was purified by Prep-TLC with EtOAc:MeOH (13:1) to give the titled Compound A51, which was separated into Compound A51a (21 mg) and Compound A51b (31 mg) by chiral Prep-HPLC. The chiral separation conditions are shown below.

| | |
|---|---|
| Column | CHIRALPAK IA |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A51a (the earlier peak): ¹H NMR (400 MHz, DMSO-d6) δ 8.82 (s, 1H), 8.04 (d, J=8.5 Hz, 1H), 7.91 (s, 1H), 7.84 (d, J=8.8 Hz, 1H), 5.85 (s, 0.5H), 5.74 (d, J=13.0 Hz, 0.5H), 4.98 (s, 0.5H), 4.72 (d, J=13.1 Hz, 0.5H), 4.40 (s, 1H), 4.31 (s, 1H), 3.97 (q, J=6.1 Hz, 1H), 3.81 (d, J=17.1 Hz, 3H), 3.63 (s, 3H), 3.30-3.28 (m, 0.5H), 2.94 (q, J=22.9, 1.5H), 2.78 (d, J=12.4 Hz, 0.5H), 2.70 (s, 3H), 2.67 (s, 0.5H), 2.32 (dd, J=23.0, 11.0 Hz, 1H), 2.02-1.87 (m, 2H), 1.50 (s, 1H), 1.36 (d, J=5.2 Hz, 3H), 1.33-1.26 (m, 1H), 0.87 (dt, J=14.7, 7.3 Hz, 3H), 0.65-0.54 (m, 3H) ppm. MS: M/e 514 (M+1)⁺.

Compound A51b (the later peak): ¹H NMR (400 MHz, DMSO-d6) δ 8.81 (s, 1H), 8.02 (d, J=8.6 Hz, 1H), 7.93 (s, 1H), 7.85 (d, J=8.6 Hz, 1H), 6.01 (d, J=13.8 Hz, 0.5H), 5.57 (s, 0.5H), 4.98 (d, J=12.5 Hz, 0.5H), 4.74 (s, 0.5H), 4.41 (s, 1H), 4.31 (s, 1H), 3.82 (d, J=13.9 Hz, 4H), 3.64 (s, 3H), 3.55 (d, J=13.5 Hz, 0.5H), 3.12 (dd, J=20.6, 10.0 Hz, 1.5H), 2.70 (s, 3H), 2.59 (d, J=8.9 Hz, 0.5H), 2.47 (s, 0.5H), 2.23 (d, J=12.4 Hz, 1H), 1.84-1.64 (m, 2H), 1.44 (s, 2H), 1.33 (d, J=5.6 Hz, 3H), 0.97 (s, 3H), 0.54 (t, J=7.3 Hz, 3H) ppm. MS: M/e 514 (M+1)⁺.

### Compound A52: 2-(6-((2S,5R)-2,5-dimethyl-4-(1-(3-methylquinoxalin-6-yl)ethyl)piperazin-1-yl)-9-ethyl-3-methyl-2-oxo-3,9-dihydro-2H-purin-8-yl)acetonitrile

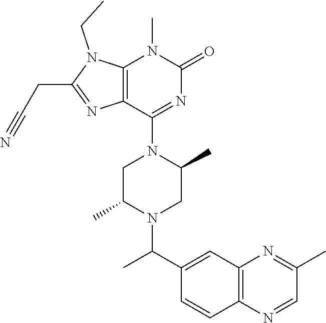 [SMILES: CCn1c(CC#N)nc2c(N3C[C@@H](C)N(C(C)c4ccc5ncc(C)nc5c4)C[C@@H]3C)nc(=O)n(C)c21]

A mixture of 2-(6-((2S,5R)-2,5-dimethylpiperazin-1-yl)-9-ethyl-3-methyl-2-oxo-3,9-dihydro-2H-purin-8-yl)acetonitrile (165 mg, 0.5 mmol), 1-(3-methylquinoxalin-6-yl)ethan-1-ol (122.2 mg, 0.65 mmol) and (cyanomethyl)trimethylphosphonium iodide (243 mg, 1 mmol) in CH₃CN (5 mL) was added DIPEA (645 mg, 5 mmol). The mixture was stirred at 100° C. overnight in a sealed tube. The reaction mixture was diluted with EtOAc (20 mL), washed with brine, dried over Na₂SO₄, concentrated. The resulting residue was purified by flash column chromatography to give the titled Compound A52 (65 mg), which was separated into Compound A52a (23 mg) and Compound A52b (28 mg) by chiral Prep-HPLC. The chiral separation conditions are shown below.

| | |
|---|---|
| Column | CHIRALPAK IH |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A52a (the earlier peak): ¹H NMR (400 MHz, CD₃OD) δ 8.79 (s, 1H), 8.06 (d, J=8.4 Hz, 1H), 8.00-7.90 (m, 2H), 6.33-5.16 (m, 2H), 4.43-4.29 (m, 2H), 4.20 (s, 2H), 3.96-3.89 (m, 1H), 3.76 (s, 3H), 3.67-3.42 (m, 1H), 3.04-2.85 (m, 3H), 2.77 (s, 3H), 1.62-1.37 (m, 9H), 0.95 (d, J=6.4 Hz, 3H) ppm. MS: M/e 500 (M+1)⁺.

Compound A52b (the later peak): ¹H NMR (400 MHz, CD₃OD) δ 8.77 (s, 1H), 8.07-7.85 (m, 3H), 6.11-4.94 (m, 2H), 4.37 (q, J=7.2 Hz, 2H), 4.22 (s, 1H), 3.76 (s, 4H), 3.71-3.42 (m, 2H), 3.30-3.10 (m, 1H), 2.76 (s, 4H), 2.20 (d, J=12 Hz, 1H), 1.54-1.36 (m, 6H), 1.37-1.22 (m, 3H), 1.10 (d, J=6.4 Hz, 3H) ppm. MS: M/e 500 (M+1)⁺.

### Compound A53: 2-(6-((2S,5R)-2,5-dimethyl-4-(1-(3-methylquinoxalin-6-yl)ethyl)piperazin-1-yl)-3-ethyl-9-methyl-2-oxo-3,9-dihydro-2H-purin-8-yl)acetonitrile

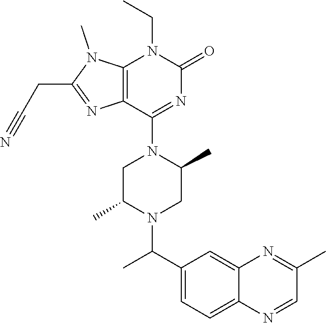 [SMILES: CCn1c(=O)nc(N2C[C@@H](C)N(C(C)c3ccc4ncc(C)nc4c3)C[C@@H]2C)c2nc(CC#N)n(C)c21]

### Step A: tert-butyl (2R,5S)-4-(3-ethyl-9-methyl-2-oxo-3,9-dihydro-2H-purin-6-yl)-2,5-dimethylpiperazine-1-carboxylate

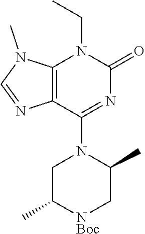 [SMILES: CCn1c(=O)nc(N2C[C@@H](C)N(C(=O)OC(C)(C)C)C[C@@H]2C)c2ncn(C)c21]

To a mixture of tert-butyl (2R,5S)-2,5-dimethyl-4-(9-methyl-2-oxo-3,9-dihydro-2H-purin-6-yl)piperazine-1-carboxylate (5 g, 13.8 mmol) in 1,4-dioxane (700 mL) and was added Cs₂CO₃ (22.5 g, 69 mmol) and CH₃CH₂I (10.7 g, 69 mmol). The reaction was stirred at room temperature overnight. The reaction mixture was filtered. The filtrate was removed under reduced pressure to dryness. The crude product was purified by flash column chromatography to give the titled compound (1.7 g, 31%). MS: M/e 391 (M+1)⁺.

### Step B: tert-butyl (2R,5S)-4-(3-ethyl-8-formyl-9-methyl-2-oxo-3,9-dihydro-2H-purin-6-yl)-2,5-dimethylpiperazine-1-carboxylate

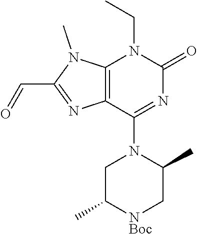 [SMILES: CCn1c(=O)nc(N2C[C@@H](C)N(C(=O)OC(C)(C)C)C[C@@H]2C)c2nc(C=O)n(C)c21]

To a solution of tert-butyl (2R,5S)-4-(3-ethyl-9-methyl-2-oxo-3,9-dihydro-2H-purin-6-yl)-2,5-dimethylpiperazine-1-carboxylate (1.7 g, 4.4 mmol) in THE (100 mL) was added LDA (6.1 mL, 2M, 13.2 mmol) at below −60° C. The reaction was stirred at −65° C. for 1 hour, then added DMF (1.56 g, 22 mmol) at −78° C. The reaction was stirred at −60° C. for another 1 hour. The reaction solvent was quenched by saturated NH₄Cl and extracted by EtOAc. The crude product was concentrated to dryness. The resulting residue was purified by flash column chromatography (DCM/MeOH) to give the titled compound (1.2 g, 66%). MS: M/e 419 (M+1)⁺.

### Step C: tert-butyl (2R,5S)-4-(3-ethyl-8-(hydroxymethyl)-9-methyl-2-oxo-3,9-dihydro-2H-purin-6-yl)-2,5-dimethylpiperazine-1-carboxylate

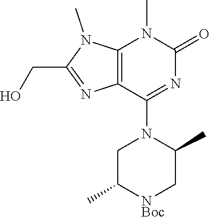 [SMILES: C[C@@H]1CN(c2nc(=O)n(C)c3c2nc(CO)n3C)[C@@H](C)CN1C(=O)OC(C)(C)C]

NaBH₄ (106 mg, 2.38 mol) was added to tert-butyl (2R,5S)-4-(3-ethyl-8-formyl-9-methyl-2-oxo-3,9-dihydro-2H-purin-6-yl)-2,5-dimethylpiperazine-1-carboxylate (1.2 g, 2.8 mmol) in MeOH (15 mL) at 0° C. for 5 min. The reaction solvent was quenched with water and extracted with DCM. The organic layer was purified by flash column chromatography (DCM/MeOH) to give the titled compound (800 mg, 66%). MS: M/e 421 (M+1)⁺.

### Step D: tert-butyl (2R,5S)-4-(8-(chloromethyl)-3-ethyl-9-methyl-2-oxo-3,9-dihydro-2H-purin-6-yl)-2,5-dimethylpiperazine-1-carboxylate

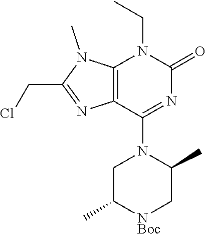 [SMILES: CCn1c(=O)nc(N2C[C@@H](C)N(C(=O)OC(C)(C)C)C[C@@H]2C)c2nc(CCl)n(C)c21]

To a solution of SOCl₂ (340 mg, 2.8 mmol) in DCM (400 mL) was added tert-butyl (2R,5S)-4-(3-ethyl-8-(hydroxymethyl)-9-methyl-2-oxo-3,9-dihydro-2H-purin-6-yl)-2,5-dimethylpiperazine-1-carboxylate (800 mg, 1.9 mmol) at −10° C. The reaction was stirred at rt for 5 min. The reaction solvent was quenched with water and washed with water, sat. NaHCO₃ and sat. NaCl. The organic layer was dried over Na₂SO₄ and used the next step directly.

### Step E: tert-butyl (2R,5S)-4-(8-(cyanomethyl)-3-ethyl-9-methyl-2-oxo-3,9-dihydro-2H-purin-6-yl)-2,5-dimethylpiperazine-1-carboxylate

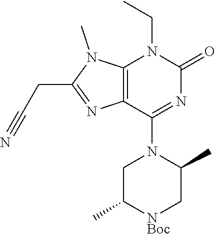 [SMILES: CCn1c(=O)nc(N2C[C@@H](C)N(C(=O)OC(C)(C)C)C[C@@H]2C)c2nc(CC#N)n(C)c21]

To the solution of tert-butyl (2R,5S)-4-(8-(chloromethyl)-3-ethyl-9-methyl-2-oxo-3,9-dihydro-2H-purin-6-yl)-2,5-dimethylpiperazine-1-carboxylate in DCM (40 mL) were added TMSCN (564 mg, 5.7 mmol) and TBAF (1M, 5.7 mL, 5.7 mmol). The resulting mixture was stirred at room temperature for 3 hours. The reaction solvent was washed by water and dried over Na₂SO₄, removed under vacuum. The crude product was purified by flash column chromatography (DCM/MeOH) to give the titled compound (500 mg, two step 61%). MS: M/e 430 (M+1)⁺.

### Step F: 2-(6-((2S,5R)-2,5-dimethylpiperazin-1-yl)-3-ethyl-9-methyl-2-oxo-3,9-dihydro-2H-purin-8-yl)acetonitrile

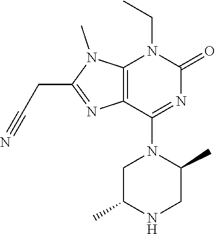 [SMILES: CCn1c(=O)nc(N2C[C@@H](C)NC[C@@H]2C)c2nc(CC#N)n(C)c21]

To a solution of tert-butyl (2R,5S)-4-(8-(cyanomethyl)-3-ethyl-9-methyl-2-oxo-3,9-dihydro-2H-purin-6-yl)-2,5-dimethylpiperazine-1-carboxylate (500 mg, 1.17 mmol) in DCM (10 mL) were added TFA (3 mL). The resulting mixture was stirred at rt for 3 hours. The reaction solvent was removed under vacuum. The crude product was dissolved with water and adjusted to pH=9 with saturated NaHCO₃. The aqueous layer was extracted with (DCM/IPA=4/1) to give the titled compound by lyophilization (400 mg, crude). MS: M/e 330 (M+1)⁺.

### Step G: 2-(6-((2S,5R)-2,5-dimethyl-4-(1-(3-methylquinoxalin-6-yl)ethyl)piperazin-1-yl)-3-ethyl-9-methyl-2-oxo-3,9-dihydro-2H-purin-8-yl)acetonitrile

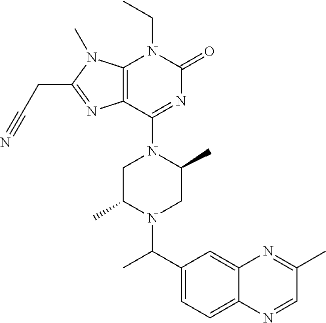 [SMILES: CCn1c(=O)nc(N2C[C@@H](C)N(C(C)c3ccc4ncc(C)nc4c3)C[C@@H]2C)c2nc(CC#N)n(C)c21]

To a solution of 2-(6-((2S,5R)-2,5-dimethylpiperazin-1-yl)-3-ethyl-9-methyl-2-oxo-3,9-dihydro-2H-purin-8-yl)acetonitrile (200 mg, 0.6 mmol) in CH₃CN (5 mL) and was added 1-(3-methylquinoxalin-6-yl)ethan-1-ol (339 mg, 1.8 mmol), (cyanomethyl)trimethylphosphonium iodide (583 mg, 2.4 mmol) and DIPEA (0.74 g, 6 mmol). The resulting mixture was stirred at 105° C. overnight. The reaction solvent was removed under reduced pressure and purified by Prep-TLC (DCM:MeOH=30: 1) to give the titled Compound A53 (50 mg), which was further separated into Compound A53a (20 mg) and Compound A53b (16 mg) by chiral Prep-SFC. The chiral separation conditions are shown below.

| | |
|---|---|
| Column | CHIRALPAK IG |
| Column Size | 3 cm × 25 cm, 5 um |
| Mobile Phase A | CO2 |
| Mobile Phase B | MeOH |
| Flow Rate | 100 mL/min |
| Wave Length | UV 220 nm |
| Temperature | 25° C. |
| Prep-SFC Equipment | Prep-SFC-150 |

Compound A53a (the earlier peak): ¹H NMR (400 MHz, CD₃OD) δ 8.79 (s, 1H), 8.06 (d, J=8.6 Hz, 1H), 8.01-7.86 (m, 2H), 6.20-5.14 (m, 2H), 4.25-4.10 (m, 4H), 3.89 (s, 4H), 3.55 (d, J=61.5 Hz, 1H), 2.90 (d, J=27.4 Hz, 3H), 2.77 (s, 3H), 1.50 (s, 3H), 1.44-1.29 (m, 6H), 0.95 (d, J=6.3 Hz, 3H) ppm. MS: M/e 500 (M+1)⁺.

Compound A53b (the later peak): ¹H NMR (400 MHz, CD₃OD) δ 8.78 (s, 1H), 8.03 (d, J=8.6 Hz, 1H), 7.99-7.87 (m, 2H), 6.01-4.98 (m, 2H), 4.32-4.26 (m, 2H), 4.21 (s, 1H), 3.97-3.82 (m, 3H), 3.82-3.47 (m, 3H), 2.76 (s, 3H), 2.71-2.66 (m, 2H), 2.19 (d, J=11.9 Hz, 1H), 1.47-1.41 (m, 3H), 1.36 (t, J=7.0 Hz, 3H), 1.29 (s, 3H), 1.10 (d, J=6.4 Hz, 3H) ppm. MS: M/e 500 (M+1)⁺.

### Compound A54: 2-(6-((2S,5R)-2,5-dimethyl-4-(1-(3-(trifluoromethyl)quinoxalin-6-yl)ethyl) piperazin-1-yl)-9-ethyl-3-methyl-2-oxo-3,9-dihydro-2H-purin-8-yl)acetonitrile

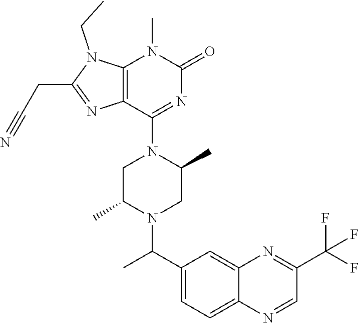 [SMILES: CCn1c(CC#N)nc2c(N3C[C@@H](C)N(C(C)c4ccc5ncc(C(F)(F)F)nc5c4)C[C@@H]3C)nc(=O)n(C)c21]

### Step A: 7-bromo-2-(trifluoromethyl)quinoxaline

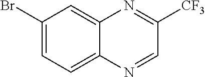 [SMILES: FC(F)(F)c1cnc2ccc(Br)cc2n1]

To a solution of 3,3-dibromo-1,1,1-trifluoropropan-2-one (5.77 g, 21.386 mmol) and AcONa (8.44 g, 106.9 mmol) in CH₃₀H (60 mL) and H₂O (60 mL) was stirred at 90° C. for 30 mins. Then 4-bromobenzene-1,2-diamine (2.0 g, 10.7 mmol) was added. The mixture was stirred at room temperature for 12 hours. After filtered, the mixture was extracted with EtOAc (35 mL×2), washed with brine, dried over Na₂SO₄, filtered, and concentrated to dryness. The resulting residue was purified by flash column chromatography (PE:EtOAc=5:1) to give the crude compound (3.2 g). The crude product (1.6 g) was further separated into the titled compound (680 mg, MS: M/e 277 (M+1)⁺) and another position isomer (620 mg, MS: M/e 277 (M+1)⁺) by chiral Prep-SFC. The chiral separation conditions are shown below.

| | |
|---|---|
| Column | CHIRALPAK IG |
| Column Size | 3 cm × 25 cm, 5 um |
| Mobile Phase A | CO₂ |
| Mobile Phase B | MeOH |
| Flow Rate | 100 mL/min |
| Wave Length | UV 220 nm |
| Temperature | 25° C. |
| Prep-SFC Equipment | Prep-SFC-150 |

### Step B: 1-(3-(trifluoromethyl)quinoxalin-6-yl)ethan-1-one

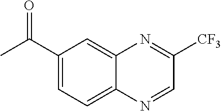 [SMILES: CC(=O)c1ccc2ncc(C(F)(F)F)nc2c1]

To a solution of 6-bromo-2-(trifluoromethyl)quinoxaline (360 mg, 1.304 mmol), tributyl(1-ethoxyvinyl)stannane (942 mg, 2.609 mmol), Pd(PPh₃)₂Cl₂ (183 mg, 0.261 mmol) in toluene (20 mL) was stirred at 90° C. under N₂ for 12 hours. The reaction mixture was quenched with saturated NaHCO₃ aq. (30 mL), extracted with EtOAc (50 mL×2), combined, washed brine (30 mL×2), dried and concentrated to dryness. The resulting oil was diluted with THE (30 mL). Then to the solution was added HCl (3 mL, 4 M in 1,4-dioxane) in drops and the mixture was stirred at room temperature for 1 hour. The reaction mixture was diluted with EtOAc (30 mL), treated with saturated NaHCO₃ aq. to pH˜8, washed with brine (30 mL×3), dried over Na₂SO₄ and concentrated to dryness. The resulting residue was purified by flash column chromatography (PE:EtOAc=6:1) to give the titled compound (300 mg, 96%). MS: M/e 241 (M+1)⁺

### Step C: 1-(3-(trifluoromethyl)quinoxalin-6-yl)ethan-1-ol

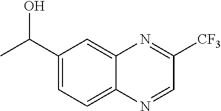 [SMILES: CC(O)c1ccc2ncc(C(F)(F)F)nc2c1]

To a solution of 1-(3-(trifluoromethyl)quinoxalin-6-yl)ethan-1-one (300 mg, 1.25 mmol) in CH₃₀H (15 mL) was added NaBH₄ (48 mg, 1.25 mmol). The reaction was stirred at room temperature for 1 hours. The reaction mixture was quenched with saturated NH₄Cl, extracted with EtOAc (35 mL×2), washed with brine, dried over Na₂SO₄, filtered, and concentrated to dryness. The resulting residue was purified by flash column chromatography (PE:EtOAc=5:1) to give the titled compound (290 mg, 96%). MS: M/e 243 (M+1)⁺

### Step D: 2-(6-((2S,5R)-2,5-dimethyl-4-(1-(3-(trifluoromethyl)quinoxalin-6-yl)ethyl)piperazin-1-yl)-9-ethyl-3-methyl-2-oxo-3,9-dihydro-2H-purin-8-yl)acetonitrile

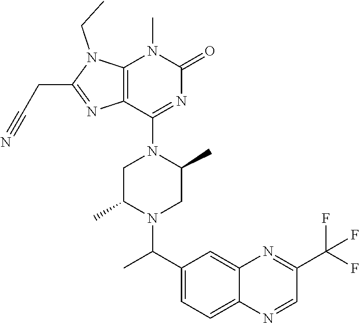 [SMILES: CCn1c(CC#N)nc2c(N3C[C@@H](C)N(C(C)c4ccc5ncc(C(F)(F)F)nc5c4)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (70 mg, 0.213 mmol), 1-(3-(trifluoromethyl)quinoxalin-6-yl)ethan-1-ol (77 mg, 0.319 mmol), (cyanomethyl)trimethylphosphonium iodide (104 mg. 0.426 mmol) and DIPEA (82 mg, 0.639 mol) in CH₃CN (1 ml). The mixture solution was degassed 3 times under N₂ atmosphere. Then the mixture solution was stirred at 100° C. for 24 hours. The reaction mixture was concentrated under reduced pressure. The crude product was purified by Prep-TLC (DCM:MeOH=15:1) to give the titled compound (56 mg, 48%). ¹H NMR (400 MHz, CD₃OD) δ 9.23 (d, J=4.1 Hz, 1H), 8.27-8.12 (m, 3H), 6.25-5.56 (m, 1H), 5.32-5.01 (m, 0.5H), 4.78-4.56 (m, 0.5H), 4.37 (q, J=7.1 Hz, 2H), 4.21 (s, 1H), 3.99 (q, J=6.3 Hz, 0.5H), 3.84 (q, J=6.3 Hz, 0.5H), 3.76 (s, 3H), 3.72-3.47 (m, 1H), 3.30-3.17 (m, 1H), 3.06-2.65 (m, 2.5H), 2.19 (d, J=12.1 Hz, 0.5H), 1.45 (dd, J=13.2, 6.5 Hz, 7H), 1.28 (s, 2H), 1.12 (d, J=6.4 Hz, 1.5H), 0.97 (d, J=6.3 Hz, 1.5H) ppm. MS: M/e 554 (M+1)⁺

### Compound A55: 2-(6-((2S,5R)-2,5-dimethyl-4-(1-(2-methylbenzo[d]thiazol-5-yl)ethyl) piperazin-1-yl)-9-ethyl-3-methyl-2-oxo-3,9-dihydro-2H-purin-8-yl)acetonitrile

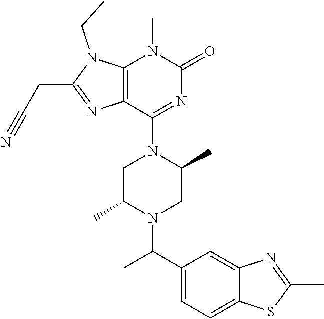 [SMILES: CCn1c(CC#N)nc2c(N3C[C@@H](C)N(C(C)c4ccc5sc(C)nc5c4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol) in CH₃CN (2 mL) and was added 1-(2-methylbenzo[d]thiazol-5-yl)ethan-1-ol (64 mg, 0.33 mmol), (cyanomethyl)trimethylphosphonium iodide (111 mg, 0.46 mmol) and DIPEA (156 mg, 1.2 mmol). The resulting mixture was stirred at 100° C. overnight. The reaction solvent was removed under reduced pressure. The resulting residue was purified by Prep-TLC (DCM:MeOH=13:1) to give the titled compound (36 mg, 24%). ¹H NMR (400 MHz, CDCl₃) δ 7.94 (d, J=17.7 Hz, 1H), 7.77 (t, J=8.6 Hz, 1H), 7.42 (t, J=7.8 Hz, 1H), 5.75-4.95 (m, 2H), 4.30 (d, J=7.3 Hz, 2H), 3.96-3.67 (m, 6H), 3.55 (d, J=6.1 Hz, 2H), 2.84 (d, J=3.1 Hz, 4H), 2.21-2.05 (m, 1H), 1.51 (d, J=6.4 Hz, 4H), 1.38-1.22 (m, 5H), 1.06-0.84 (m, 3H) ppm. MS: M/e 460 (M+1)⁺.

### Compound A56: 2-(6-((2S,5R)-2,5-dimethyl-4-(1-(2-methylbenzo[d]thiazol-6-yl)ethyl) piperazin-1-yl)-9-ethyl-3-methyl-2-oxo-3,9-dihydro-2H-purin-8-yl)acetonitrile

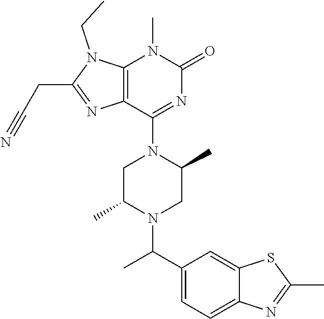 [SMILES: CCn1c(CC#N)nc2c(N3C[C@@H](C)N(C(C)c4ccc5nc(C)sc5c4)C[C@@H]3C)nc(=O)n(C)c21]

### Step A: 1-(2-methylbenzo[d]thiazol-6-yl)ethan-1-ol

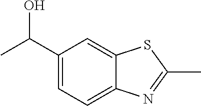

To a solution of 1-(2-methylbenzo[d]thiazol-6-yl)ethan-1-one (150 mg, 0.79 mmol) in MeOH (15 mL) was added NaBH₄ (30 mg, 0.79 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (130 mg, 86%). MS: M/e 194 (M+1)⁺.

### Step B: 2-(6-((2S,5R)-2,5-dimethyl-4-(1-(2-methylbenzo[d]thiazol-6-yl)ethyl)piperazin-1-yl)-9-ethyl-3-methyl-2-oxo-3,9-dihydro-2H-purin-8-yl)acetonitrile

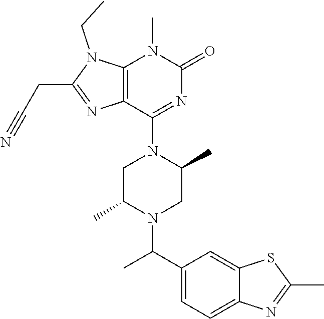 [SMILES: CCn1c(CC#N)nc2c(N3C[C@@H](C)N(C(C)c4ccc5nc(C)sc5c4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol) in CH₃CN (3 mL) and was added 1-(2-methylbenzo[d]thiazol-6-yl)ethan-1-ol (29 mg, 0.15 mmol), (cyanomethyl)trimethylphosphonium iodide (110 mg, 0.46 mmol) and DIPEA (98 mg, 0.76 mmol). The resulting mixture was stirred at 105° C. overnight. The reaction solvent was removed under reduced pressure to dryness. The resulting residue was purified by Prep-HPLC (Method A) to give the titled Compound A56a (26 mg) and Compound A56b (24 mg).

Compound A56a (the earlier peak): ¹H NMR (400 MHz, CD₃OD) δ 7.92 (s, 2H), 7.62 (s, 1H), 6.38-4.98 (m, 2H), 4.37 (d, J=7.2 Hz, 2H), 4.24 (s, 2H), 4.11-3.83 (m, 1H), 3.77 (s, 3H), 3.73-3.32 (m, 2H), 3.29-3.05 (m, 2H), 2.84 (s, 3H), 1.93-1.41 (m, 6H), 1.40-1.04 (m, 6H) ppm. MS: M/e 505 (M+1)⁺

Compound A56b (the later peak): ¹H NMR (400 MHz, CD₃OD) δ 8.41-7.83 (m, 2H), 7.81-7.52 (m, 1H), 6.74-5.95 (m, 1H), 5.95-5.17 (m, 1H), 4.84-4.48 (m, 2H), 4.36 (s, 2H), 4.26 (s, 2H), 3.78 (s, 3H), 3.73-3.36 (m, 2H), 3.10-2.95 (m, 1H), 2.87 (s, 3H), 1.82 (s, 3H), 1.66-1.35 (m, 6H), 1.35-1.13 (m, 3H) ppm. MS: M/e 505 (M+1)⁺

### Compound A57: 2-(6-((2S,5R)-2,5-dimethyl-4-(1-(2-methyl-1H-benzo[d]imidazol-5-yl)ethyl)piperazin-1-yl)-9-ethyl-3-methyl-2-oxo-3,9-dihydro-2H-purin-8-yl)acetonitrile

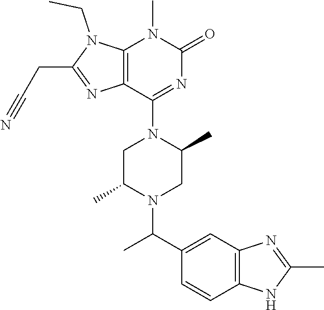 [SMILES: CCn1c(CC#N)nc2c(N3C[C@@H](C)N(C(C)c4ccc5[nH]c(C)nc5c4)C[C@@H]3C)nc(=O)n(C)c21]

### Step A: mixture of 6-bromo-2-methyl-1-((2-(trimethylsilyl)ethoxy)methyl)-1H-benzo[d]imidazole and 5-bromo-2-methyl-1-((2-(trimethylsilyl)ethoxy)methyl)-1H-benzo[d]imidazole

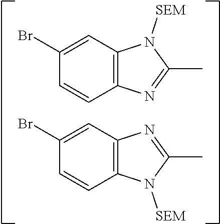

To a solution of 5-bromo-2-methyl-1H-benzo[d]imidazole (1.05 g, 5 mmol) in THE (20 mL) was added NaH (60%, 400 mg, 10 mmol) at 0° C. After 30 minutes, (2-(chloromethoxy)ethyl)trimethylsilane (1 g, 6 mmol) in THE (5 ml) was added to the reaction and the mixture was stirred at RT for 2 hours. The reaction mixture was quenched with H₂O and extracted with EtOAc. The combined organic layers were washed with brine, dried over Na₂SO₄, filtered, and concentrated to dryness. The resulting residue was purified by flash column chromatography (DCM:MeOH=10:1) to give the titled compound (1.5 g, 88%). MS: M/e 341 (M+1)⁺.

### Step B: mixture of 1-(2-methyl-1-((2-(trimethylsilyl)ethoxy)methyl)-1H-benzo[d]imidazol-6-yl)ethan-1-one and 1-(2-methyl-1-((2-(trimethylsilyl)ethoxy)methyl)-1H-benzo[d]imidazol-5-yl)ethan-1-one

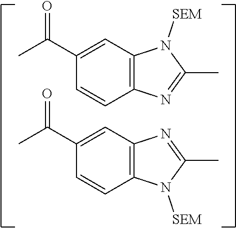

A mixture of 6-bromo-2-methyl-1-((2-(trimethylsilyl)ethoxy)methyl)-1H-benzo[d]imidazole and 5-bromo-2-methyl-1-((2-(trimethylsilyl)ethoxy)methyl)-1H-benzo[d]imidazole (1.5 g, 4.4 mmol), tributyl(1-ethoxyvinyl)stannane (1.9 g, 5.3 mmol) and Pd(PPh₃)₂Cl₂ (308 mg, 0.44 mmol) in toluene (20 mL) was stirred at 100° C. under N₂ overnight. The reaction mixture was concentrated under reduced pressure. The residue was diluted with THE (20 mL) followed by addition of TFA (1.5 mL) and stirred for 30 minutes. The resulting mixture was washed with saturated NaHCO₃ aq., extracted with EtOAc, dried and concentrated to dryness. The resulting oil was purified by flash column chromatography (DCM:MeOH=10:1) to give the titled compound (500 mg, 37%) as a mixture. MS: M/e 305 (M+1)⁺.

### Step C: mixture of 1-(2-methyl-1-((2-(trimethylsilyl)ethoxy)methyl)-1H-benzo[d]imidazol-6-yl)ethan-1-ol and 1-(2-methyl-1-((2-(trimethylsilyl)ethoxy)methyl)-1H-benzo[d]imidazol-5-yl)ethan-1-ol

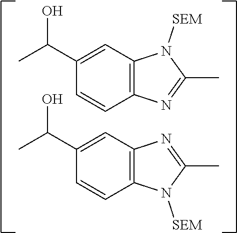

To a solution of the mixture of 1-(2-methyl-1-((2-(trimethylsilyl)ethoxy)methyl)-1H-benzo[d]imidazol-6-yl)ethan-1-one and 1-(2-methyl-1-((2-(trimethylsilyl)ethoxy)methyl)-1H-benzo[d]imidazol-5-yl)ethan-1-one (200 mg, 0.67 mmol) in EtOH (4 mL) was added NaBH₄ (25 mg, 0.67 mmol). The mixture was stirred at room temperature for 1 hour. The mixture was treated with water, extracted with DCM. The combined organic layers were dried over MgSO₄ and concentrated to dryness. The resulting residue (200 mg, crude) was used in the next step without further purification. MS: M/e 307 (M+1)⁺.

### Step D: mixture of 2-(6-((2S,5R)-2,5-dimethyl-4-(1-(2-methyl-1-((2-(trimethylsilyl)ethoxy)methyl)-1H-benzo[d]imidazol-5-yl)ethyl)piperazin-1-yl)-9-ethyl-3-methyl-2-oxo-3,9-dihydro-2H-purin-8-yl)acetonitrile and 2-(6-((2S,5R)-2,5-dimethyl-4-(1-(2-methyl-1-((2-(trimethylsilyl)ethoxy)methyl)-1H-benzo[d]imidazol-6-yl)ethyl)piperazin-1-yl)-9-ethyl-3-methyl-2-oxo-3,9-dihydro-2H-purin-8-yl)acetonitrile

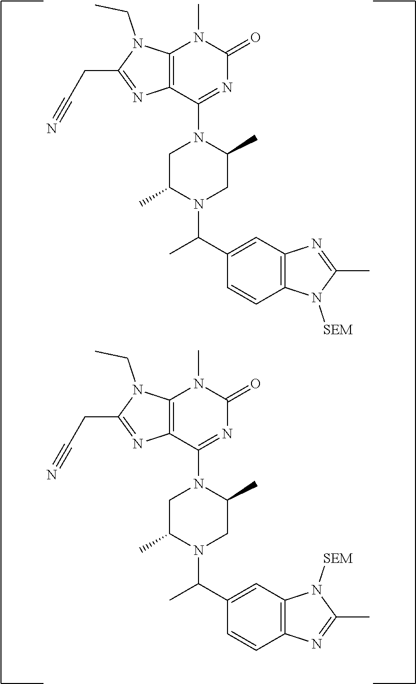

To a solution of the mixture of 1-(2-methyl-1-((2-(trimethylsilyl)ethoxy)methyl)-1H-benzo[d]imidazol-6-yl)ethan-1-ol and 1-(2-methyl-1-((2-(trimethylsilyl)ethoxy)methyl)-1H-benzo[d]imidazol-5-yl)ethan-1-ol (100 mg, 0.33 mmol), 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol), (cyanomethyl)trimethylphosphonium iodide (145 mg, 0.6 mmol) and DIPEA (155 mg, 1.2 mmol) in MeCN (3 mL) was stirred at 100° C. overnight in a sealed tube. The mixture was diluted with EA (20 mL), washed with brine (10 mL×3), dried, concentrated. The resulting residue was purified by Prep-TLC (CH₂Cl₂:MeOH=13:1) to give the titled compound (20 mg, 11%) as a mixture. MS: M/e 618 (M+1)⁺.

### Step E: 2-(6-((2S,5R)-2,5-dimethyl-4-(1-(2-methyl-1H-benzo[d]imidazol-5-yl)ethyl)piperazin-1-yl)-9-ethyl-3-methyl-2-oxo-3,9-dihydro-2H-purin-8-yl)acetonitrile

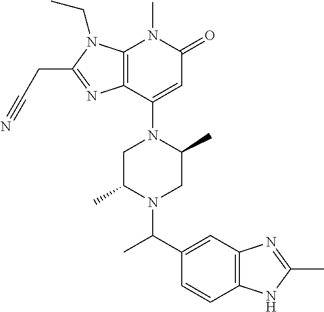 [SMILES: CCn1c(CC#N)nc2c(N3C[C@@H](C)N(C(C)c4ccc5[nH]c(C)nc5c4)C[C@@H]3C)cc(=O)n(C)c21]

To a stirred mixture of 2-(6-((2S,5R)-2,5-dimethyl-4-(1-(2-methyl-1-((2-(trimethylsilyl)ethoxy)methyl)-1H-benzo[d]imidazol-5-yl)ethyl)piperazin-1-yl)-9-ethyl-3-methyl-2-oxo-3,9-dihydro-2H-purin-8-yl)acetonitrile and 2-(6-((2S,5R)-2,5-dimethyl-4-(1-(2-methyl-1-((2-(trimethylsilyl)ethoxy)methyl)-1H-benzo[d]imidazol-6-yl)ethyl)piperazin-1-yl)-9-ethyl-3-methyl-2-oxo-3,9-dihydro-2H-purin-8-yl)acetonitrile (20 mg) in DCM (1 mL) was added TFA (1 mL). The resulting mixture was stirred at room temperature overnight. The reaction mixture was concentrated to dryness. The resulting residue was purified by Prep-HPLC(Method A) to give the titled compound (2 mg, 12%). ¹H NMR (400 MHz, CD₃OD) δ 7.49-7.40 (m, 2H), 7.26 (t, J=8.6 Hz, 1H), 5.90 (s, 2H), 4.37 (d, J=7.0 Hz, 2H), 3.76 (s, 3H), 3.68-3.57 (m, 2H), 2.90 (s, 2H), 2.65 (s, 2H), 2.55 (d, J=3.6 Hz, 3H), 2.25 (d, J=12.0 Hz, 1H), 1.46 (t, J=6.9 Hz, 4H), 1.37 (dd, J=10.3, 6.5 Hz, 3H), 1.30-1.14 (m, 2H), 1.06-0.88 (m, 3H) ppm. MS: M/e 488 (M+1)⁺.

### Compound A58: 2-(6-((2S,5R)-4-(1-(2,3-dihydrobenzo[b][1,4]dioxin-6-yl)ethyl)-2,5-dimethyl piperazin-1-yl)-3,9-dimethyl-2-oxo-3,9-dihydro-2H-purin-8-yl)acetonitrile

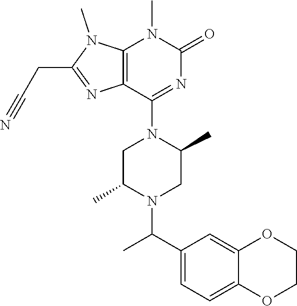 [SMILES: CC(c1ccc2c(c1)OCCO2)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (50 mg, 0.159 mol), 1-(2,3-dihydrobenzo[b][1,4]dioxin-6-yl)ethan-1-ol (43 mg, 0.238 mol), (cyanomethyl)trimethylphosphonium iodide (77 mg. 0.318 mol) and DIPEA (62 mg, 0.477 mol) in CH₃CN (1 ml). The mixture solution was degassed 3 times under N₂ atmosphere. Then the mixture solution was stirred at 105° C. for 24 hours. The reaction mixture was concentrated under reduced pressure. The resulting residue was purified by Prep-TLC (DCM:MeOH=15:1) to give the titled Compound A58 (30 mg), which was further separated into Compound A58a (12 mg) and Compound A58b (12 mg) by Prep-HPLC (Method A).

Compound A58: ¹H NMR (400 MHz, CDCl₃) δ 7.69-7.44 (m, 0.5H), 7.30-7.27 (m, 1H), 6.91-6.68 (m, 1.5H), 6.21-4.42 (m, 2H), 4.29-4.23 (m, 4H), 4.06-3.86 (m, 4H), 3.80 (d, J=9.6 Hz, 3H), 3.55-3.40 (m, 2H), 3.35-3.04 (m, 0.5H), 3.01-2.52 (m, 1.5H), 1.95-1.72 (m, 2H), 1.47-1.32 (m, 3H), 1.25 (s, 3H), 1.10-0.75 (m, 3H) ppm. MS: M/e 478 (M+1)⁺

Compound A58a (the earlier peak): ¹H NMR (400 MHz, CDCl₃) δ 7.53-7.33 (m, 1H), 7.22-6.70 (m, 2H), 6.28-5.17 (m, 2H), 4.32-4.23 (m, 4H), 3.91 (d, J=27.8 Hz, 3H), 3.79 (s, 3H), 3.56-3.51 (m, 2H), 3.23-2.62 (m, 2H), 2.10-1.95 (m, 2H), 1.48-1.36 (m, 3H), 1.25 (s, 3H), 1.20-0.93 (m, 3H) ppm. MS: M/e 478 (M+1)⁺

Compound A58b (the later peak): ¹H NMR (400 MHz, CDCl₃) δ 7.52-7.34 (m, 1H), 7.10-6.78 (m, 2H), 6.15-5.80 (m, 0.5H), 5.53-5.13 (m, 1H), 4.59-4.54 (m, 0.5H), 4.29-4.24 (m, 4H), 4.05-3.90 (m, 4H), 3.79 (s, 3H), 3.59-3.18 (m, 2H), 3.07-2.42 (m, 2H), 2.09-1.82 (m, 2H), 1.54-1.37 (m, 3H), 1.31 (s, 3H), 1.26-0.82 (m, 3H) ppm. MS: M/e 478 (M+1)⁺.

### Compound A59: 2-(6-((2S,5R)-4-(1-(3,3-dimethyl-2,3-dihydrobenzo[b][1,4]dioxin-6-yl)ethyl)-2,5-dimethylpiperazin-1-yl)-3,9-dimethyl-2-oxo-3,9-dihydro-2H-purin-8-yl)acetonitrile

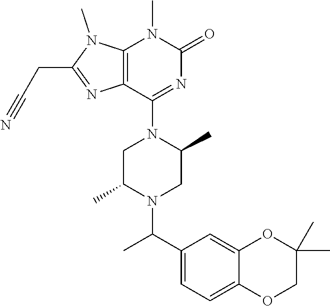 [SMILES: CC(c1ccc2c(c1)OC(C)(C)CO2)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (60 mg, 0.190 mol), 1-(3,3-dimethyl-2,3-dihydrobenzo[b][1,4]dioxin-6-yl)ethan-1-ol (80 mg, 0.381 mol), (cyanomethyl)trimethylphosphonium iodide (93 mg. 0.381 mol) and DIPEA (147 mg, 1.143 mol) in CH₃CN (1 ml). The mixture solution was degassed 3 times under N₂ atmosphere. Then the mixture solution was stirred at 105° C. for 24 hours. The reaction mixture was concentrated under reduced pressure. The resulting residue was purified by Prep-TLC (DCM:MeOH=15:1) to give the titled Compound A59 (30 mg), which was further separated into Compound A59a (8 mg) and Compound A59b (8 mg) by Prep-HPLC (Method A).

Compound A59a (the earlier peak): ¹H NMR (400 MHz, CD₃OD) δ 6.92-6.71 (m, 3H), 5.93-5.76 (m, 1H), 5.10-5.05 (m, 0.5H), 4.57-4.50 (m, 0.5H), 4.23-4.16 (m, 0.5H), 3.93 (s, 3H), 3.88 (s, 2H), 3.77 (s, 3H), 3.75-3.53 (m, 1.5H), 3.49-3.43 (m, 2H), 2.93-2.54 (m, 2H), 2.33 (d, J=12.3 Hz, 1H), 1.34 (d, J=6.6 Hz, 3H), 1.31 (d, J=1.5 Hz, 6H), 1.26 (d, J=6.6 Hz, 3H), 1.05 (d, J=6.4 Hz, 3H) ppm. MS: M/e 506 (M+1)⁺

Compound A59b (the later peak): ¹H NMR (400 MHz, CD₃OD) δ 6.89-6.76 (m, 3H), 6.10-5.47 (m, 1H), 5.35-4.56 (m, 1H), 4.20 (d, J=5.0 Hz, 0.5H), 3.93 (s, 3H), 3.89 (s, 2H), 3.78 (s, 3H), 3.69-3.60 (m, 1H), 3.48-3.34 (m, 1.5H), 3.13-2.64 (m, 4H), 1.43 (d, J=6.4 Hz, 3H), 1.34 (d, J=8.8 Hz, 9H), 0.95 (d, J=6.4 Hz, 3H) ppm. MS: M/e 506 (M+1)⁺

### Compound A60: 2-(6-((2S,5R)-4-(1-(3,3-dimethyl-2,3-dihydrobenzo[b][1,4]dioxin-6-yl)ethyl)-2,5-dimethylpiperazin-1-yl)-9-ethyl-3-methyl-2-oxo-3,9-dihydro-2H-purin-8-yl)acetonitrile

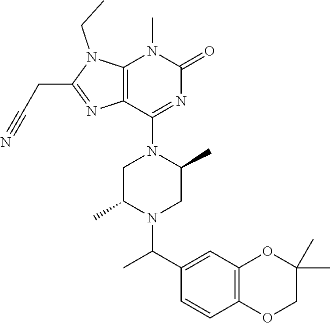 [SMILES: CCn1c(CC#N)nc2c(N3C[C@@H](C)N(C(C)c4ccc5c(c4)OC(C)(C)CO5)C[C@@H]3C)nc(=O)n(C)c21]

A mixture of 2-(6-((2S,5R)-2,5-dimethylpiperazin-1-yl)-9-ethyl-3-methyl-2-oxo-3,9-dihydro-2H-purin-8-yl)acetonitrile (165 mg, 0.5 mmol), 1-(3,3-dimethyl-2,3-dihydrobenzo[b][1,4]dioxin-6-yl)ethan-1-ol (270 mg, 1.3 mmol) and (cyanomethyl)trimethylphosphonium iodide (243 mg, 1 mmol) in CH₃CN (5 mL) was added DIPEA (645 mg, 5 mmol). The mixture was stirred at 100° C. overnight in a sealed tube. The reaction mixture was diluted with EtOAc (20 mL), washed with brine, dried over Na₂SO₄, concentrated. The resulting residue was purified by flash column chromatography (DCM:MeOH=15:1) to give the titled Compound A60 (30 mg), which was separated into Compound A60a (9 mg) and Compound A60b (12 mg) by chiral Prep-HPLC. The chiral separation conditions are shown below.

| | |
|---|---|
| Column | CHIRALPAK IH |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A60a (the earlier peak): ¹H NMR (400 MHz, CD₃OD) δ 6.84-6.74 (m, J=6.4 Hz, 3H), 6.13-5.04 (m, 2H), 4.60-4.52 (m, 0.5H), 4.36 (q, J=7.2 Hz, 2H), 4.25-4.17 (m, 0.5H), 3.89 (s, 2H), 3.76 (s, 3H), 3.67-3.41 (m, 2H), 3.06-2.67 (m, 4H), 1.50-1.39 (m, 6H), 1.35-1.23 (m, 9H), 0.93 (d, J=6 Hz, 3H) ppm. MS: M/e 520 (M+1)⁺.

Compound A60b (the later peak): ¹H NMR (400 MHz, CD₃OD) δ 6.84-6.74 (m, 3H), 6.02-5.00 (m, 2H), 4.37 (q, J=7.2 Hz, 2H), 3.87 (s, 2H), 3.76 (s, 3H), 3.70-3.31 (m, 4H), 3.09-2.52 (m, 2H), 2.26 (d, J=11.6 Hz, 1H), 1.46 (t, J=7.2 Hz, 3H), 1.34-1.21 (m, 12H), 1.02 (d, J=6 Hz, 3H) ppm. MS: M/e 500 (M+1)⁺.

### Compound A61: 2-(6-((2S,5R)-4-(1-(3,3-dimethyl-2,3-dihydrobenzo[b][1,4]dioxin-6-yl)ethyl)-2,5-dimethylpiperazin-1-yl)-3-ethyl-9-methyl-2-oxo-3,9-dihydro-2H-purin-8-yl)acetonitrile

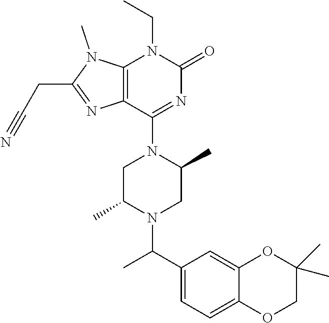 [SMILES: CCn1c(=O)nc(N2C[C@@H](C)N(C(C)c3ccc4c(c3)OC(C)(C)CO4)C[C@@H]2C)c2nc(CC#N)n(C)c21]

To a solution of 2-(6-((2S,5R)-2,5-dimethylpiperazin-1-yl)-3-ethyl-9-methyl-2-oxo-3,9-dihydro-2H-purin-8-yl)acetonitrile (330 mg, 1 mmol) in CH₃CN (5 mL) and was added 1-(3,3-dimethyl-2,3-dihydrobenzo[b][1,4]dioxin-6-yl)ethan-1-ol (624 mg, 3 mmol), (cyanomethyl)trimethylphosphonium iodide (972 mg, 4 mmol) and DIPEA (1.3 g, 10 mmol). The resulting mixture was stirred at 105° C. overnight. The reaction solvent was removed under reduced pressure and purified by Prep-TLC (DCM:MeOH=30:1) to give the titled Compound A61 (120 mg), which was further separated into Compound A61a (55 mg) and Compound A61b (40 mg) by chiral Prep-SFC. The chiral separation conditions are shown below.

| | |
|---|---|
| Column | CHIRALPAK IG |
| Column Size | 3 cm × 25 cm, 5 um |
| Mobile Phase A | CO2 |
| Mobile Phase B | MeOH |
| Flow Rate | 100 mL/min |
| Wave Length | UV 220 nm |
| Temperature | 25° C. |
| Prep-SFC Equipment | Prep-SFC-150 |

Compound A61a (the earlier peak): ¹H NMR (400 MHz, CD₃OD) δ 6.80 (d, J=3.0 Hz, 3H), 6.25-5.14 (m, 2H), 4.28 (q, J=6.9 Hz, 2H), 3.89 (d, J=3.7 Hz, 5H), 3.54-3.33 (m, 3H), 2.95-2.90 (m, 2H), 2.89-2.80 (m, 2H), 1.43 (d, J=5.9 Hz, 3H), 1.39-1.30 (m, 9H), 1.26 (d, J=6.4 Hz, 3H), 0.87 (d, J=6.5 Hz, 3H) ppm. MS: M/e 520 (M+1)⁺.

Compound A61b (the later peak): ¹H NMR (400 MHz, CD₃OD) δ 6.85-6.70 (m, 3H), 6.01-4.99 (m, 2H), 4.28 (q, J=7.2 Hz, 2H), 3.93-3.86 (m, 5H), 3.75-3.70 (m, 1H), 3.51 (d, J=25.8 Hz, 1H), 3.35-3.33 (m, 2H), 2.79 (s, 1H), 2.58 (s, 1H), 2.25 (d, J=12.3 Hz, 1H), 1.41-1.16 (m, 15H), 1.01 (d, J=6.4 Hz, 3H) ppm. MS: M/e 520 (M+1)⁺.

### Compound A62: 2-(6-((2S,5R)-4-(1-(3,3-dimethyl-2,3-dihydrobenzo[b][1,4]dioxin-6-yl)ethyl)-2,5-dimethylpiperazin-1-yl)-3,9-diethyl-2-oxo-3,9-dihydro-2H-purin-8-yl)acetonitrile

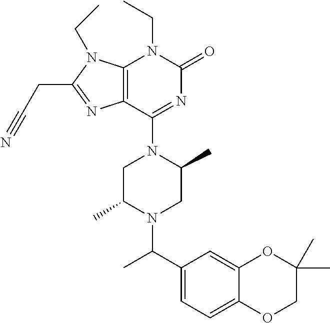 [SMILES: CCn1c(CC#N)nc2c(N3C[C@@H](C)N(C(C)c4ccc5c(c4)OC(C)(C)CO5)C[C@@H]3C)nc(=O)n(CC)c21]

### Step A: 1-ethyl-6-(ethylamino)pyrimidine-2,4 (1H,3H)-dione

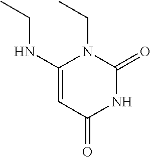

To a stirred mixture of 6-chloro-1-methylpyrimidine-2,4 (1H,3H)-dione (35 g, 0.2 mol) in EtOH (200 mL) was added aq. CH₃CH₂NH₂ (w/w=50%, 36 g, 0.4 mol). After the addition, the reaction mixture was stirred at 65° C. overnight. The reaction mixture was cooled to room temperature and filtered. The caked was collected and dried to give the titled compound (35 g, 96%) as a white solid. MS: M/e 184 (M+1)⁺.

### Step B: 1-ethyl-6-(ethylamino)-5-nitrosopyrimidine-2,4 (1H,3H)-dione

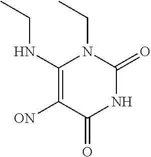

To a stirred mixture of 1-ethyl-6-(ethylamino)pyrimidine-2,4 (1H,3H)-dione (35 g, 0.19 mol) in H₂O (400 mL) was added NaNO₂ (14.5 g, 0.21 mol) at 0˜5° C. Then AcOH (34.2 g, 0.57 mol) was added dropwise at that temperature. After then, the mixture was stirred for an hour. The reaction mixture was filtered. The resulting cake was collected and dried to give the titled compound, which was directly used to the next step. MS: M/e 213 (M+1)⁺.

### Step C: 5-amino-1-ethyl-6-(ethylamino)-4-hydroxypyrimidin-2 (1H)-one

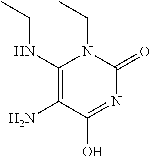

To a stirred mixture of 1-ethyl-6-(ethylamino)-5-nitrosopyrimidine-2,4 (1H,3H)-dione (crude, 0.19 mol) in H₂O (400 mL) was added Na₂S₂O₄ (99.2 g, 0.57 mmol) portionwise at 20˜30° C. After the addition, the reaction mixture was stirred for 30 minutes and cooled in an ice-water bath for several hours and filtered. The cake was collected, dried to give the titled compound (17 g, 45%) as a white solid. MS: M/e 199 (M+1)⁺.

### Step D: 8-((benzyloxy)methyl)-3,9-diethyl-6-hydroxy-3,9-dihydro-2H-purin-2-one

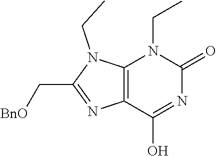 [SMILES: CCn1c(COCc2ccccc2)nc2c(O)nc(=O)n(CC)c21]

A mixture of 5-amino-1-ethyl-6-(ethylamino)-4-hydroxypyrimidin-2 (1H)-one (17 g, 86 mmol) in 2-(benzyloxy)acetic acid (15 g, 90 mmol) was stirred at 240° C. for 1.5 hour. The mixture was allowed cool to room temperature and dissolved in aq.NaOH (2.0 M, 100 mL), then acidified to pH=5˜6 with aq.HCl and filtered. The cake was collected, dried to give the titled compound (17 g, 60%) as a light-yellow solid. MS: M/e 329 (M+1)⁺.

### Step E: tert-butyl (2R,5S)-4-(8-((benzyloxy)methyl)-3,9-diethyl-2-oxo-3,9-dihydro-2H-purin-6-yl)-2,5-dimethylpiperazine-1-carboxylate

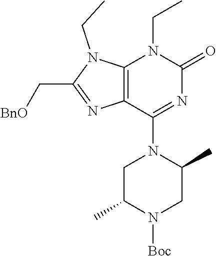 [SMILES: CCn1c(COCc2ccccc2)nc2c(N3C[C@@H](C)N(C(=O)OC(C)(C)C)C[C@@H]3C)nc(=O)n(CC)c21]

To a stirred mixture of 8-((benzyloxy)methyl)-3,9-diethyl-6-hydroxy-3,9-dihydro-2H-purin-2-one (4.28 g, 20 mmol) in CH₃CN (50 mL) was added BOP (3.06 g, 20 mmol), followed by DBU (8.8 g, 20 mmol). After the addition, the reaction was stirred overnight. The reaction mixture was concentrated to give the residue, which was dissolved in EtOAc (150 mL), washed with H₂O (20 mL), brine, dried over Na₂SO₄, concentrated. The resulting residue was purified by flash column chromatography to give the titled compound (1.8 g, 34%) as a white solid. MS: M/e 525 (M+1)⁺.

### Step F: tert-butyl (2R,5S)-4-(3,9-diethyl-8-(hydroxymethyl)-2-oxo-3,9-dihydro-2H-purin-6-yl)-2,5-dimethylpiperazine-1-carboxylate

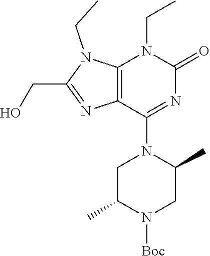 [SMILES: CCn1c(CO)nc2c(N3C[C@@H](C)N(C(=O)OC(C)(C)C)C[C@@H]3C)nc(=O)n(CC)c21]

To a stirred solution of tert-butyl (2R,5S)-4-(8-((benzyloxy)methyl)-3,9-diethyl-2-oxo-3,9-dihydro-2H-purin-6-yl)-2,5-dimethylpiperazine-1-carboxylate (1.8 g, 3.43 mmol) in MeOH/AcOH (100 mL/2 mL) was added Pd/C (500 mg). After the addition, the reaction mixture was stirred for 2 days under H₂ (1 atm). The reaction mixture was filtered, and filtrate was concentrated to give the titled compound (1.5 g, 100%) as a whited solid. MS: M/e 435 (M+1)⁺.

### Step G: tert-butyl (2R,5S)-4-(8-(cyanomethyl)-3,9-diethyl-2-oxo-3,9-dihydro-2H-purin-6-yl)-2,5-dimethylpiperazine-1-carboxylate

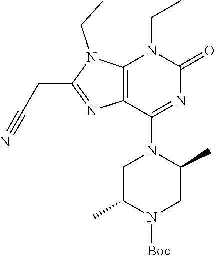 [SMILES: CCn1c(CC#N)nc2c(N3C[C@@H](C)N(C(=O)OC(C)(C)C)C[C@@H]3C)nc(=O)n(CC)c21]

To a stirred solution of tert-butyl (2R,5S)-4-(3,9-diethyl-8-(hydroxymethyl)-2-oxo-3,9-dihydro-2H-purin-6-yl)-2,5-dimethylpiperazine-1-carboxylate (crude, 3.24 mmol) in CH₂Cl₂ (15 mL) was added SOCl₂ (0.41 g, 3.4 mmol). After stirred for 10 min, the reaction mixture was concentrated and dissolved in CH₂Cl₂ (10 mL), TBAF (1.0 M, 6.48 mL, 6.48 mmol) was added followed by TMSCN (0.64 g, 6.48 mmol). After the addition, the reaction mixture was stirred overnight. The reaction mixture was concentrated to give the residue, treated with H₂O and extracted with EtOAc (40 mL×2). The combined organic layers were washed with brine, dried over Na₂SO₄, concentrated to dryness. The resulting residue was purified by flash column chromatography to give the titled compound (0.9 g, 63%) as a white solid. MS: M/e 444 (M+1)⁺.

### Step H: 2-(6-((2S,5R)-2,5-dimethylpiperazin-1-yl)-3,9-diethyl-2-oxo-3,9-dihydro-2H-purin-8-yl)acetonitrile

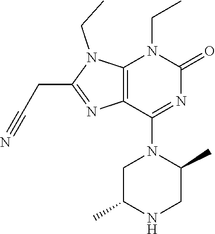 [SMILES: CCn1c(CC#N)nc2c(N3C[C@@H](C)NC[C@@H]3C)nc(=O)n(CC)c21]

To a stirred solution of tert-butyl (2R,5S)-4-(8-(cyanomethyl)-3,9-diethyl-2-oxo-3,9-dihydro-2H-purin-6-yl)-2,5-dimethylpiperazine-1-carboxylate (0.9 g, 2.02 mmol) in CH₂Cl₂ (10 mL) was added TFA (2 mL). After then, the reaction mixture was stirred for 2 hours. The reaction mixture was concentrated to give the residue, which was basified to pH=9˜10 with K₂CO₃ and extracted with CH₂Cl₂/IPA (3/1, 40 mL×5). The combined organic layers were dried over Na₂SO₄, concentrated, further lyophilized to give the titled compound (0.9 g, 100%), which was directly used to the next step. MS: M/e 342 (M+1)⁺.

### Step I: 2-(6-((2S,5R)-4-(1-(3,3-dimethyl-2,3-dihydrobenzo[b][1,4]dioxin-6-yl)ethyl)-2,5-dimethylpiperazin-1-yl)-3,9-diethyl-2-oxo-3,9-dihydro-2H-purin-8-yl)acetonitrile

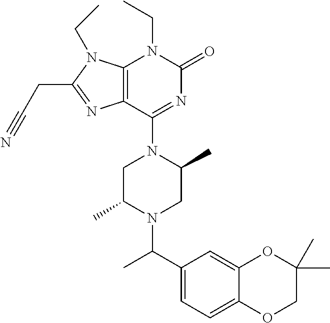 [SMILES: CCn1c(CC#N)nc2c(N3C[C@@H](C)N(C(C)c4ccc5c(c4)OC(C)(C)CO5)C[C@@H]3C)nc(=O)n(CC)c21]

A mixture of 2-(6-((2S,5R)-2,5-dimethylpiperazin-1-yl)-3,9-diethyl-2-oxo-3,9-dihydro-2H-purin-8-yl)acetonitrile (1 g, 2.92 mmol), 1-(3,3-dimethyl-2,3-dihydrobenzo[b][1,4]dioxin-6-yl)ethan-1-ol (0.91 g, 4.4 mmol), (cyanomethyl)trimethylphosphonium iodide (1.42 g, 5.84 mmol) and DIPEA (1.9 g, 14.6 mmol) in CH₃CN (20 mL) was stirred overnight at 100° C. in a sealed tube. The reaction mixture was concentrated to give the residue, treated with H₂O and extracted with EtOAc (30 mL×2). The combined organic layers were washed with brine, dried over Na₂SO₄, concentrated. The resulting residue was purified by flash column chromatography to give the titled compound (230 mg, 15%). ¹H NMR (400 MHz, CD3OD) δ 6.90-6.75 (m, 3H), 6.21-4.98 (m, 2H), 4.33-4.17 (m, 6H), 3.88 (d, J=2.4 Hz, 2H), 3.78-3.68 (m, 1H), 3.63-3.41 (m, 1.5H), 3.27-3.19 (m, 1H), 3.06-2.76 (m, 1.5H), 1.46 (t, J=7.2 Hz, 3H), 1.40-1.34 (m, 6H), 1.34-1.29 (m, 9H), 1.08-0.84 (m, 3H) ppm. MS: M/e 534 (M+1)⁺.

### Compound A63: 2-(6-((2S,5R)-4-(1-(3,3-dimethyl-2,3-dihydrobenzo[b][1,4]dioxin-6-yl)ethyl)-5-ethyl-2-methylpiperazin-1-yl)-3,9-dimethyl-2-oxo-3,9-dihydro-2H-purin-8-yl)acetonitrile

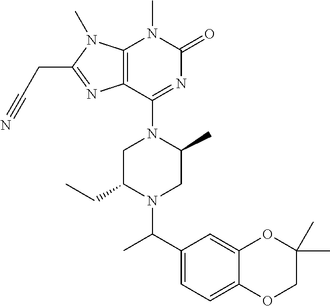 [SMILES: CC[C@@H]1CN(c2nc(=O)n(C)c3c2nc(CC#N)n3C)[C@@H](C)CN1C(C)c1ccc2c(c1)OC(C)(C)CO2]

### Step A: tert-butyl (2R,5S)-4-(2-chloro-9H-purin-6-yl)-2-ethyl-5-methylpiperazine-1-carboxylate

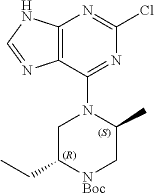

To a solution of 2,6-dichloro-9H-purine (8.0 g, 42.33 mmol) and tert-butyl (2R,5S)-2-ethyl-5-methylpiperazine-1-carboxylate (10.6 g, 46.56 mmol) in EtOH (130 mL) was added DIPEA (10.92 g, 84.66 mmol). The reaction was stirred at 80° C. for 12 hours. The reaction mixture was concentrated under reduced pressure to give the title compound (16.0 g, 99%). MS: M/e 381 (M+1)⁺.

### Step B: tert-butyl (2R,5S)-4-(2-chloro-9-methyl-9H-purin-6-yl)-2-ethyl-5-methylpiperazine-1-carboxylate

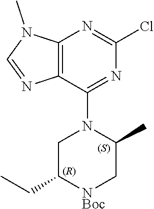

To a solution of tert-butyl (2R,5S)-4-(2-chloro-9H-purin-6-yl)-2-ethyl-5-methylpiperazine-1-carboxylate (16.0 g, 42.11 mmol) and K₂CO₃ (11.62 g, 84.21 mmol) in acetone (150 mL) was added CH₃I (12.0 g, 84.21 mmol). The reaction was stirred at room temperature for 16 hours. The reaction mixture was diluted with water, extracted with EtOAc (300 mL×2), washed with brine, dried over Na₂SO₄, filtered, and concentrated to dryness. The resulting residue was purified by flash column chromatography (EtOAc:PE=1:1) to give the titled compound (14 g, 84%). MS: M/e 395 (M+1)⁺.

### Step C: tert-butyl (2R,5S)-2-ethyl-4-(2-hydroxy-9-methyl-9H-purin-6-yl)-5-methylpiperazine-1-carboxylate

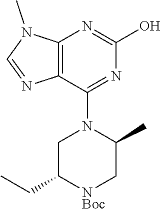 [SMILES: CC[C@@H]1CN(c2nc(O)nc3c2ncn3C)[C@@H](C)CN1C(=O)OC(C)(C)C]

To a solution of tert-butyl (2R,5S)-4-(2-chloro-9-methyl-9H-purin-6-yl)-2-ethyl-5-methylpiperazine-1-carboxylate (14.0 g, 35.53 mmol), Pd₂(dba)₃ (3.26 g, 3.56 mmol), t-BuXphos (3.03 g, 7.11 mmol) and KOH (35.6 mL, 3M, 106.06 mmol) in dioxane (120 mL) was degassed 3 times under N₂ atmosphere. Then the mixture was stirred at 90° C. for 12 hours. The reaction mixture was diluted with water and extracted with EtOAc (90 mL×2). The combined organic layers were washed with brine, dried over Na₂SO₄, filtered and concentrated to dryness. The resulting residue was purified by flash column chromatography (DCM:MeOH=10:1) to give the titled compound (13 g, 97%). MS: M/e 377 (M+1)⁺.

### Step D: tert-butyl (2R,5S)-4-(3,9-dimethyl-2-oxo-3,9-dihydro-2H-purin-6-yl)-2-ethyl-5-methylpiperazine-1-carboxylate

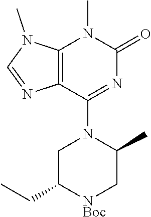 [SMILES: CC[C@@H]1CN(c2nc(=O)n(C)c3c2ncn3C)[C@@H](C)CN1C(=O)OC(C)(C)C]

To a solution of tert-butyl (2R,5S)-2-ethyl-4-(2-hydroxy-9-methyl-9H-purin-6-yl)-5-methylpiperazine-1-carboxylate (13 g, 34.6 mmol) and Cs₂CO₃ (33.8 g, 103.7 mmol) in dioxane (180 mL) was added CH₃I (14.7 g, 103.7 mmol). The reaction was stirred at room temperature for 16 hours. The reaction mixture was diluted with water, extracted with EtOAc (150 mL×2), washed with brine, dried over Na₂SO₄, filtered, and concentrated to dryness. The resulting residue was purified by flash column chromatography (DCM:MeOH=20:1) to give the titled compound (9.5 g, 70%). MS: M/e 391 (M+1)⁺.

### Step E: tert-butyl (2R,5S)-2-ethyl-4-(8-formyl-3,9-dimethyl-2-oxo-3,9-dihydro-2H-purin-6-yl)-5-methylpiperazine-1-carboxylate

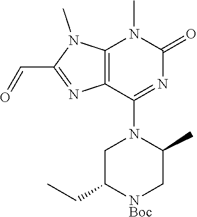 [SMILES: CC[C@@H]1CN(c2nc(=O)n(C)c3c2nc(C=O)n3C)[C@@H](C)CN1C(=O)OC(C)(C)C]

To a solution of tert-butyl (2R,5S)-4-(3,9-dimethyl-2-oxo-3,9-dihydro-2H-purin-6-yl)-2-ethyl-5-methylpiperazine-1-carboxylate (9.5 g, 24.35 mmol) in THE (150 mL) was degassed 3 times under N₂ atmosphere. Then added LDA (49 mL, 2M, 97.44 mmol) dropwise at −78° C. The mixture was stirred at −78° C. for 1 hours. Added DMF (8.9 g, 121.75 mmol) to quench the reaction solution. The reaction mixture was stirred for 1 hour and diluted with saturated NH₄Cl, extracted with EtOAc (100 mL×2), washed with brine, dried over Na₂SO₄, filtered, and concentrated to dryness. The resulting residue was purified by flash column chromatography (DCM:MeOH=20:1) to give the titled compound (6.8 g, 67%). MS: M/e 419 (M+1)⁺.

### Step F: tert-butyl (2R,5S)-2-ethyl-4-(8-(hydroxymethyl)-3,9-dimethyl-2-oxo-3,9-dihydro-2H-purin-6-yl)-5-methylpiperazine-1-carboxylate

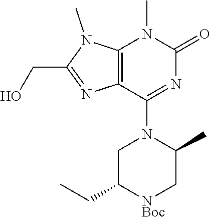 [SMILES: CC[C@@H]1CN(c2nc(=O)n(C)c3c2nc(CO)n3C)[C@@H](C)CN1C(=O)OC(C)(C)C]

To a solution of tert-butyl (2R,5S)-2-ethyl-4-(8-formyl-3,9-dimethyl-2-oxo-3,9-dihydro-2H-purin-6-yl)-5-methylpiperazine-1-carboxylate (3.7 g, 8.831 mmol) in MeOH (30 mL) was added NaBH₄ (671 mg, 17.66 mmol). The reaction was stirred at room temperature for 1 hours. The reaction mixture was quenched with saturated NH₄Cl, extracted with EtOAc (50 mL×2), washed with brine, dried over Na₂SO₄, filtered, and concentrated to dryness. The resulting residue was purified by flash column chromatography (DCM:MeOH=10:1) to give the titled compound (2.8 g, 75%). MS: M/e 421 (M+1)⁺.

### Step G: tert-butyl (2R,5S)-4-(8-(chloromethyl)-3,9-dimethyl-2-oxo-3,9-dihydro-2H-purin-6-yl)-2-ethyl-5-methylpiperazine-1-carboxylate

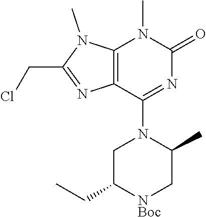 [SMILES: CC[C@@H]1CN(c2nc(=O)n(C)c3c2nc(CCl)n3C)[C@@H](C)CN1C(=O)OC(C)(C)C]

To a solution of tert-butyl (2R,5S)-2-ethyl-4-(8-(hydroxymethyl)-3,9-dimethyl-2-oxo-3,9-dihydro-2H-purin-6-yl)-5-methylpiperazine-1-carboxylate (1.6 g, 3.80 mmol) in DCM (20 mL) was added SOCl₂ (497 mg, 4.181 mmol). The reaction was stirred at room temperature for 0.5 hours. The reaction mixture concentrated to give the titled compound (1.62 g, 97%). MS: M/e 439 (M+1)⁺.

### Step H: tert-butyl (2R,5S)-4-(8-(cyanomethyl)-3,9-dimethyl-2-oxo-3,9-dihydro-2H-purin-6-yl)-2-ethyl-5-methylpiperazine-1-carboxylate

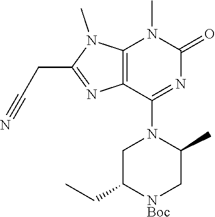 [SMILES: CC[C@@H]1CN(c2nc(=O)n(C)c3c2nc(CC#N)n3C)[C@@H](C)CN1C(=O)OC(C)(C)C]

To a solution of tert-butyl (2R,5S)-4-(8-(chloromethyl)-3,9-dimethyl-2-oxo-3,9-dihydro-2H-purin-6-yl)-2-ethyl-5-methylpiperazine-1-carboxylate (1.62 g, 3.7 mmol) in DCM (25 mL) was added TBAF (7.4 mL, 1M, 7.4 mmol), followed TMSCN (734 mg, 7.4 mmol). The reaction was stirred at room temperature for 16 hours. The reaction mixture was diluted with water, extracted with DCM (40 mL×2), washed with brine, dried over Na₂SO₄, filtered, and concentrated to dryness. The resulting residue was purified by flash column chromatography (PE:EtOAc=1:5) to give the titled compound (1.3 g, 82%). MS: M/e 430 (M+1)⁺.

### Step I: 2-(6-((2S,5R)-5-ethyl-2-methylpiperazin-1-yl)-3,9-dimethyl-2-oxo-3,9-dihydro-2H-purin-8-yl)acetonitrile

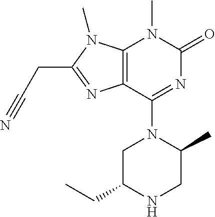 [SMILES: CC[C@@H]1CN(c2nc(=O)n(C)c3c2nc(CC#N)n3C)[C@@H](C)CN1]

To a stirred solution of tert-butyl (2R,5S)-4-(8-(cyanomethyl)-3,9-dimethyl-2-oxo-3,9-dihydro-2H-purin-6-yl)-2-ethyl-5-methylpiperazine-1-carboxylate (1.3 g, 3.03 mmol) in CH₂Cl₂ (30 mL) was added TFA (6 mL). Then reaction mixture was stirred at room temperature for 4 hours, concentrated to give a residue, basified to pH=10˜11 with saturated NaHCO₃ aq., and extracted with CH₂Cl₂/IPA (3/1, 50 mL×3). The combined organic layers were washed with brine, dried over Na₂SO₄, and concentrated to give the titled compound (800 mg, 80%). MS: M/e 330 (M+1)⁺.

### Step J: 2-(6-((2S,5R)-4-(1-(3,3-dimethyl-2,3-dihydrobenzo[b][1,4]dioxin-6-yl)ethyl)-5-ethyl-2-methylpiperazin-1-yl)-3,9-dimethyl-2-oxo-3,9-dihydro-2H-purin-8-yl)acetonitrile

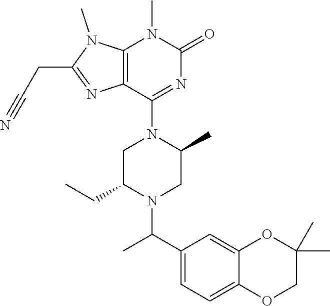 [SMILES: CC[C@@H]1CN(c2nc(=O)n(C)c3c2nc(CC#N)n3C)[C@@H](C)CN1C(C)c1ccc2c(c1)OC(C)(C)CO2]

A solution of 2-(6-((2S,5R)-5-ethyl-2-methylpiperazin-1-yl)-3,9-dimethyl-2-oxo-3,9-dihydro-2H-purin-8-yl)acetonitrile (420 mg, 1.276 mmol), 1-(3,3-dimethyl-2,3-dihydrobenzo[b][1,4]dioxin-6-yl)ethan-1-ol (531 mg, 2.552 mmol), (cyanomethyl)trimethylphosphonium iodide (620 mg. 2.552 mmol) and DIPEA (494 mg, 3.828 mol) in CH₃CN (2 ml). The mixture solution was degassed 3 times under N₂ atmosphere. Then the mixture solution was stirred at 105° C. for 24 hours. The reaction mixture was concentrated under reduced pressure. The resulting residue was purified by Prep-TLC (DCM:MeOH=15:1) to give the titled Compound A63 (260 mg), which was further separated into Compound A63a (87 mg) and Compound A63b (68 mg) by Prep-HPLC (Method A).

Compound A63a (the earlier peak): ¹H NMR (400 MHz, CD₃OD) δ 6.85-6.72 (m, 3H), 6.10-5.73 (m, 1H), 5.18-5.12 (m, 0.5H), 4.83-4.77 (m, 0.5H), 3.91 (s, 2H), 3.85 (s, 3H), 3.78 (s, 3H), 3.63-3.54 (m, 1H), 3.42-3.34 (m, 0.5H), 3.30-3.24 (m, 2H), 3.12-3.06 (m, 0.5H), 2.80 (s, 2H), 2.53-2.46 (m, 1H), 1.48-1.36 (m, 4H), 1.32 (d, J=3.1 Hz, 6H), 1.29 (d, J=6.3 Hz, 4H), 0.72 (t, J=7.3 Hz, 3H) ppm. MS: M/e 520 (M+1)⁺

Compound A63b (the later peak): ¹H NMR (400 MHz, CD₃OD) δ 6.86-6.66 (m, 3H), 6.16-5.76 (m, 1H), 5.04-4.95 (m, 1H), 3.92 (s, 3H), 3.87 (s, 2H), 3.78 (s, 3H), 3.69-3.57 (m, 0.5H), 3.48-3.40 (m, 1H), 3.30-3.24 (m, 2H), 3.14-3.05 (m, 1H), 2.85-2.45 (m, 1.5H), 2.27 (d, J=12.1 Hz, 1H), 1.51-1.39 (m, 2H), 1.31 (d, J=1.5 Hz, 6H), 1.25-1.19 (m, 6H), 1.00 (t, J=7.3 Hz, 3H) ppm. MS: M/e 520 (M+1)⁺

### Compound A64: 2-(6-((2S,5R)-4-(1-(3,3-dimethyl-2,3-dihydrobenzo[b][1,4]dioxin-6-yl)ethyl)-2,5-diethylpiperazin-1-yl)-3,9-dimethyl-2-oxo-3,9-dihydro-2H-purin-8-yl)acetonitrile

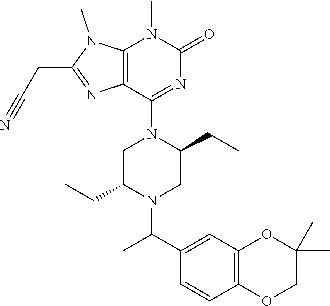 [SMILES: CC[C@H]1CN(C(C)c2ccc3c(c2)OC(C)(C)CO3)[C@H](CC)CN1c1nc(=O)n(C)c2c1nc(CC#N)n2C]

### Step A: tert-butyl (2R,5S)-4-(8-((benzyloxy)methyl)-3,9-dimethyl-2-oxo-3,9-dihydro-2H-purin-6-yl)-2,5-diethylpiperazine-1-carboxylate

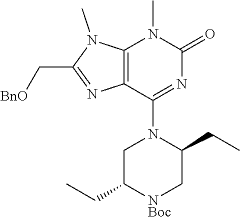 [SMILES: CC[C@@H]1CN(c2nc(=O)n(C)c3c2nc(COCc2ccccc2)n3C)[C@@H](CC)CN1C(=O)OC(C)(C)C]

To a solution of 8-((benzyloxy)methyl)-6-hydroxy-3,9-dimethyl-3,9-dihydro-2H-purin-2-one (3.85 g, 12.83 mmol), tert-butyl (2R,5S)-2,5-diethylpiperazine-1-carboxylate (6.21 g, 25.66 mmol) and BOP (11.35 g, 25.66 mmol) in CH₃CN (100 mL) was added DBU (3.91 g, 25.66 mmol). The reaction mixture was diluted with water, extracted with EtOAc (150 mL×2), washed with brine, dried over Na₂SO₄, filtered, and concentrated to dryness. The resulting residue was purified by flash column chromatography (PE:EtOAc=3:1) to give the titled compound (4.7 g, 70%). MS: M/e 525 (M+1)⁺.

### Step B: tert-butyl (2R,5S)-2,5-diethyl-4-(8-(hydroxymethyl)-3,9-dimethyl-2-oxo-3,9-dihydro-2H-purin-6-yl)piperazine-1-carboxylate

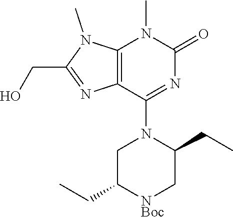 [SMILES: CC[C@@H]1CN(c2nc(=O)n(C)c3c2nc(CO)n3C)[C@@H](CC)CN1C(=O)OC(C)(C)C]

To a solution of tert-butyl (2R,5S)-4-(8-((benzyloxy)methyl)-3,9-dimethyl-2-oxo-3,9-dihydro-2H-purin-6-yl)-2,5-diethylpiperazine-1-carboxylate (4.7 g, 8.97 mmol) and Pd/C (800 mg, 10% in water) in MeOH (60 mL) at room temperature was added AcOH (2 mL). The resulting mixture was degassed 3 times under H₂ atmosphere and stirred at room temperature under H₂ atmosphere for 36 hours. After filtration, the combined organic layers were concentrated under reduced pressure to give the titled compound (3.2 g, 82%). MS: M/e 435 (M+1)⁺.

### Step C: tert-butyl (2R,5S)-4-(8-(chloromethyl)-3,9-dimethyl-2-oxo-3,9-dihydro-2H-purin-6-yl)-2,5-diethylpiperazine-1-carboxylate

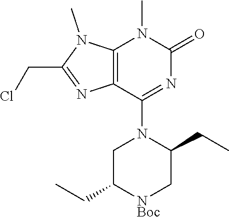 [SMILES: CC[C@@H]1CN(c2nc(=O)n(C)c3c2nc(CCl)n3C)[C@@H](CC)CN1C(=O)OC(C)(C)C]

To a solution of tert-butyl (2R,5S)-2,5-diethyl-4-(8-(hydroxymethyl)-3,9-dimethyl-2-oxo-3,9-dihydro-2H-purin-6-yl)piperazine-1-carboxylate (1.84 g, 4.24 mmol) in DCM (30 mL) was added SOCl₂ (555 mg, 4.66 mmol). The reaction was stirred at room temperature for 0.5 hours. The reaction mixture concentrated to give the titled compound (1.9 g, 99%). MS: M/e 453 (M+1)⁺.

### Step D: tert-butyl (2R,5S)-4-(8-(cyanomethyl)-3,9-dimethyl-2-oxo-3,9-dihydro-2H-purin-6-yl)-2,5-diethylpiperazine-1-carboxylate

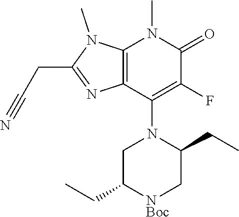 [SMILES: CC[C@@H]1CN(c2c(F)c(=O)n(C)c3c2nc(CC#N)n3C)[C@@H](CC)CN1C(=O)OC(C)(C)C]

To a solution of tert-butyl (2R,5S)-4-(8-(chloromethyl)-3,9-dimethyl-2-oxo-3,9-dihydro-2H-purin-6-yl)-2,5-diethylpiperazine-1-carboxylate (1.90 g, 4.20 mmol) in DCM (40 mL) was added TBAF (8.4 mL, 1M, 8.4 mmol), followed TMSCN (834 mg, 8.4 mmol). The reaction was stirred at room temperature for 16 hours. The reaction mixture was diluted with water, extracted with DCM (40 mL×2), washed with brine, dried over Na₂SO₄, filtered, and concentrated to dryness. The resulting residue was purified by flash column chromatography (PE:EtOAc=1:5) to give the titled compound (1.3 g, 70%). MS: M/e 444 (M+1)⁺.

### Step E: 2-(6-((2S,5R)-2,5-diethylpiperazin-1-yl)-3,9-dimethyl-2-oxo-3,9-dihydro-2H-purin-8-yl)acetonitrile

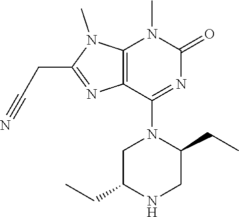 [SMILES: CC[C@@H]1CN(c2nc(=O)n(C)c3c2nc(CC#N)n3C)[C@@H](CC)CN1]

To a stirred solution of tert-butyl (2R,5S)-4-(8-(cyanomethyl)-3,9-dimethyl-2-oxo-3,9-dihydro-2H-purin-6-yl)-2,5-diethylpiperazine-1-carboxylate (1.3 g, 2.935 mmol) in CH₂Cl₂ (30 mL) was added TFA (5 mL). Then reaction mixture was stirred at room temperature for 4 hours, concentrated to give a residue, basified to pH=10˜11 with saturated NaHCO₃ aq., and extracted with CH₂Cl₂/IPA (3/1, 50 mL×3). The combined organic layers were washed with brine, dried over Na₂SO₄, and concentrated to give the titled compound (820 mg, 82%). MS: M/e 344 (M+1)⁺.

### Step F: 2-(6-((2S,5R)-4-(1-(3,3-dimethyl-2,3-dihydrobenzo[b][1,4]dioxin-6-yl)ethyl)-2,5-diethylpiperazin-1-yl)-3,9-dimethyl-2-oxo-3,9-dihydro-2H-purin-8-yl)acetonitrile

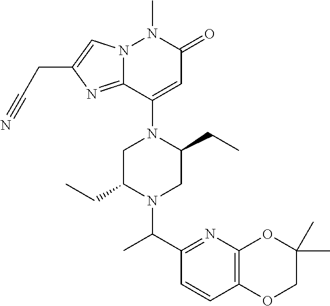 [SMILES: CC[C@H]1CN(C(C)c2ccc3c(n2)OC(C)(C)CO3)[C@H](CC)CN1c1cc(=O)n(C)n2cc(CC#N)nc12]

A solution of 2-(6-((2S,5R)-2,5-diethylpiperazin-1-yl)-3,9-dimethyl-2-oxo-3,9-dihydro-2H-purin-8-yl)acetonitrile (450 mg, 1.312 mmol), 1-(3,3-dimethyl-2,3-dihydrobenzo[b][1,4]dioxin-6-yl)ethan-1-ol (546 mg, 2.624 mmol), (cyanomethyl)trimethylphosphonium iodide (638 mg. 2.624 mmol) and DIPEA (508 mg, 3.936 mol) in CH₃CN (2 ml). The mixture solution was degassed 3 times under N₂ atmosphere. Then the mixture solution was stirred at 105° C. for 24 hours. The reaction mixture was concentrated under reduced pressure. The resulting residue was purified by Prep-TLC (DCM:MeOH=15:1) to give the titled Compound A64 (240 mg), which was further separated into Compound A64a (68 mg) and Compound A64b (54 mg) by Prep-HPLC (Method A).

Compound A64a (the earlier peak): ¹H NMR (400 MHz, CD₃OD) δ 6.87-6.70 (m, 3H), 5.93 (s, 0.5H), 5.84 (d, J=13.3 Hz, 0.5H), 5.07 (s, 0.5H), 4.83 (s, 0.5H), 3.91 (d, J=7.7 Hz, 3H), 3.87 (s, 2H), 3.77 (s, 3H), 3.60-3.53 (m, 1H), 3.40-3.32 (m, 1.5H), 3.06-2.89 (m, 1.5H), 2.84-2.65 (m, 1H), 2.49-2.36 (m, 1H), 2.10-1.87 (m, 2H), 1.49-1.38 (m, 1H), 1.32 (d, J=3.6 Hz, 7H), 1.28 (d, J=6.4 Hz, 4H), 0.92 (t, J=7.3 Hz, 3H), 0.68 (dt, J=13.9, 7.0 Hz, 3H) ppm. MS: M/e 534 (M+1)⁺

Compound A64b (the later peak): ¹H NMR (400 MHz, CD₃OD) δ 6.82 (s, 1H), 6.80-6.74 (m, 2H), 6.09 (d, J=13.5 Hz, 0.5H), 5.69 (s, 0.5H), 5.08 (d, J=13.3 Hz, 0.5H), 4.81 (s, 0.5H), 3.92 (d, J=3.7 Hz, 3H), 3.87 (d, J=1.6 Hz, 2H), 3.77 (s, 3H), 3.59-3.52 (m, 0.5H), 3.47-3.38 (m, 1H), 3.20 (d, J=13.1 Hz, 0.5H), 3.12-3.03 (m, 1H), 2.53-3.35 (m, 2H), 1.89-1.70 (m, 2H), 1.51-1.38 (m, 2H), 1.31 (d, J=8.0 Hz, 8H), 1.25 (d, J=6.7 Hz, 3H), 0.98 (t, J=5.8 Hz, 3H), 0.67 (dd, J=8.6, 5.4 Hz, 3H) ppm. MS: M/e 534 (M+1)⁺

### Compound A65: 2-(6-((2S,5R)-4-(1-(3,3-dimethyl-2,3-dihydrobenzo[b][1,4]dioxin-6-yl)ethyl)-5-ethyl-2-methylpiperazin-1-yl)-9-ethyl-3-methyl-2-oxo-3,9-dihydro-2H-purin-8-yl)acetonitrile

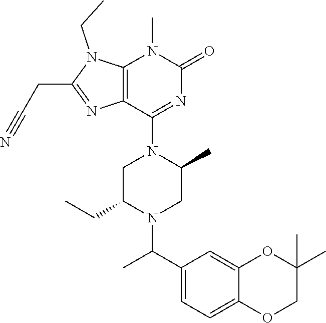 [SMILES: CC[C@@H]1CN(c2nc(=O)n(C)c3c2nc(CC#N)n3CC)[C@@H](C)CN1C(C)c1ccc2c(c1)OC(C)(C)CO2]

A solution of 2-(9-ethyl-6-((2S,5R)-5-ethyl-2-methylpiperazin-1-yl)-3-methyl-2-oxo-3,9-dihydro-2H-purin-8-yl)acetonitrile (50 mg, 0.15 mmol), 1-(3,3-dimethyl-2,3-dihydrobenzo[b][1,4]dioxin-6-yl)ethan-1-ol (30 mg, 0.15 mmol), (cyanomethyl)trimethylphosphonium iodide (106 mg, 0.44 mmol) and DIPEA (94 mg, 0.73 mmol) in MeCN (3 ml) was stirred at 100° C. overnight. After completed, the solution was concentrated under reduced pressure to dryness. The resulting residue was purified by Prep-HPLC (Method A) to give the titled Compound A65 (50 mg, 50%). ¹H NMR (400 MHz, CD₃OD) δ 6.91-6.72 (m, 3H), 6.42-5.58 (m, 1H), 5.33-4.88 (m, 1H), 4.36 (q, J=7.0 Hz, 2H), 4.23 (s, 1H), 3.89 (d, J=8.7 Hz, 2H), 3.76 (s, 3H), 3.71-3.48 (m, 1H), 3.47-3.31 (m, 1H), 3.25-3.08 (m, 1H), 3.02-2.87 (m, 1H), 2.79-2.33 (m, 2H), 1.52-1.34 (m, 8H), 1.34-1.20 (m, 9H), 1.04-0.75 (m, 3H) ppm. MS: M/e 534 (M+1)⁺

### Compound A66: 2-(6-((2S,5R)-4-(1-(3,3-dimethyl-2,3-dihydrobenzo[b][1,4]dioxin-6-yl)ethyl)-5-ethyl-2-methylpiperazin-1-yl)-3-ethyl-9-methyl-2-oxo-3,9-dihydro-2H-purin-8-yl)acetonitrile

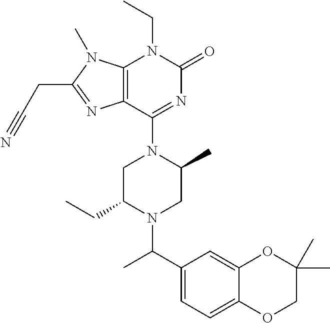 [SMILES: CC[C@@H]1CN(c2nc(=O)n(CC)c3c2nc(CC#N)n3C)[C@@H](C)CN1C(C)c1ccc2c(c1)OC(C)(C)CO2]

### Step A: 1-ethyl-6-(methylamino)pyrimidine-2,4 (1H,3H)-dione

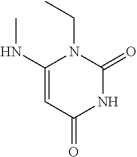

To a stirred mixture of 6-chloro-1-methylpyrimidine-2,4 (1H,3H)-dione (10 g, 57.5 mmol) in EtOH (120 mL) was added aq. CH₃NH₂ (w/w=33%, 16 g, 0.174 mol). After the addition, the reaction mixture was stirred at 65° C. overnight. The reaction mixture was cooled to room temperature and filtered. The caked was collected and dried to give the titled compound (10 g) as a white solid, which was directly used to the next step. MS: M/e 170 (M+1)⁺.

### Step B: 1-ethyl-6-(methylamino)-5-nitrosopyrimidine-2,4 (1H,3H)-dione

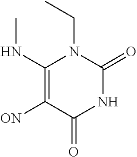

To a stirred mixture of 1-ethyl-6-(methylamino)pyrimidine-2,4 (1H,3H)-dione (10 g, 0.059 mol) in H₂O (100 mL) was added NaNO₂ (4.5 g, 0.065 mol) at 0˜5° C. Then AcOH (10.6 g, 0.177 mol) was added dropwise at that temperature. After then, the mixture was stirred for 1.5 hour. The reaction mixture was filtered. The resulting cake was collected and dried to give the titled compound (7.2 g, 84%), which was directly used to the next step. MS: M/e 199 (M+1)⁺.

### Step C: 5-amino-1-ethyl-4-hydroxy-6-(methylamino)pyrimidin-2 (1H)-one

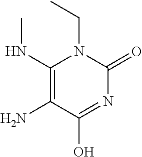

To a stirred mixture of 1-ethyl-6-(ethylamino)-5-nitrosopyrimidine-2,4 (1H,3H)-dione (7.2, 0.036 mol) in H₂O (70 mL) was added Na₂S₂O₄ (18.7 g, 0.107 mol) portionwise at 20-30° C. After the addition, the reaction mixture was stirred for 30 minutes and cooled in an ice-water bath for several hours and filtered. The cake was collected, dried to give the titled compound (17 g, 45%) as a white solid, which was directly used to the next step. MS: M/e 1 (M+1)⁺.

### Step D: 8-((benzyloxy)methyl)-3-ethyl-6-hydroxy-9-methyl-3,9-dihydro-2H-purin-2-one

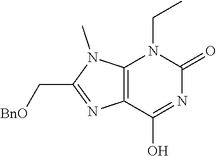 [SMILES: CCn1c(=O)nc(O)c2nc(COCc3ccccc3)n(C)c21]

A mixture of 5-amino-1-ethyl-6-(ethylamino)-4-hydroxypyrimidin-2 (1H)-one (2.5 g, 13.5 mmol) in 2-(benzyloxy)acetic acid (2.5 g, 14.9 mmol) was stirred at 240° C. for 1.5 hour. The mixture was allowed cool to room temperature and dissolved in aq.NaOH (2.0 M, 40 mL), then acidified to pH=5˜6 with aq.HCl and filtered. The cake was collected, dried to give the titled compound (2.8 g, 66%) as a light-yellow solid, which was directly used to the next step. MS: M/e 315 (M+1)⁺.

### Step E: tert-butyl (2R,5S)-4-(8-((benzyloxy)methyl)-3-ethyl-9-methyl-2-oxo-3,9-dihydro-2H-purin-6-yl)-2-ethyl-5-methylpiperazine-1-carboxylate

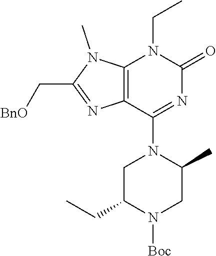 [SMILES: CC[C@@H]1CN(c2nc(=O)n(CC)c3c2nc(COCc2ccccc2)n3C)[C@@H](C)CN1C(=O)OC(C)(C)C]

To a stirred mixture of 8-((benzyloxy)methyl)-3-ethyl-6-hydroxy-9-methyl-3,9-dihydro-2H-purin-2-one (1 g, 3.2 mmol) in CH₃CN (10 mL) was added tert-butyl tert-butyl (2R,5S)-2-ethyl-5-methylpiperazine-1-carboxylate (1.36 g, 6.4 mmol), BOP (2.8 g, 6.4 mmol), followed by DBU (1 g, 6.4 mmol). After the addition, the reaction was stirred overnight. The reaction mixture was concentrated to give the residue, which was dissolved in EtOAc (50 mL), washed with H₂O (20 mL), brine, dried over Na₂SO₄, concentrated. The resulting residue was purified by flash column chromatography to give the titled compound (1.6 g, 95%) as a white solid. MS: M/e 525 (M+1)⁺.

### Step F: tert-butyl (2R,5S)-2-ethyl-4-(3-ethyl-8-(hydroxymethyl)-9-methyl-2-oxo-3,9-dihydro-2H-purin-6-yl)-5-methylpiperazine-1-carboxylate

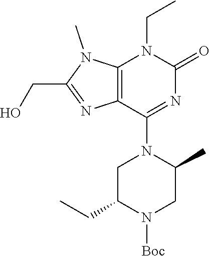 [SMILES: CC[C@@H]1CN(c2nc(=O)n(CC)c3c2nc(CO)n3C)[C@@H](C)CN1C(=O)OC(C)(C)C]

To a stirred solution of tert-butyl (2R,5S)-4-(8-((benzyloxy)methyl)-3-ethyl-9-methyl-2-oxo-3,9-dihydro-2H-purin-6-yl)-2-ethyl-5-methylpiperazine-1-carboxylate (1.6 g, 3.1 mmol) in MeOH/AcOH (15 mL/0.5 mL) was added Pd/C (500 mg). After the addition, the reaction mixture was stirred for 2 days under H₂ (1 atm). The reaction mixture was filtered. The resulting filtrate was concentrated to give the titled compound (1.3 g, 81%) as a whited solid, which was directly used to the next step. MS: M/e 435 (M+1)⁺.

### Step G: tert-butyl (2R,5S)-4-(8-(cyanomethyl)-3-ethyl-9-methyl-2-oxo-3,9-dihydro-2H-purin-6-yl)-2-ethyl-5-methylpiperazine-1-carboxylate

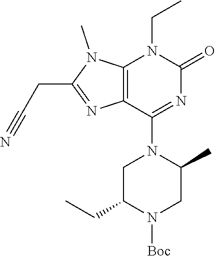 [SMILES: CC[C@@H]1CN(c2nc(=O)n(CC)c3c2nc(CC#N)n3C)[C@@H](C)CN1C(=O)OC(C)(C)C]

To a stirred solution of tert-butyl (2R,5S)-2-ethyl-4-(3-ethyl-8-(hydroxymethyl)-9-methyl-2-oxo-3,9-dihydro-2H-purin-6-yl)-5-methylpiperazine-1-carboxylate (3.3 g, 7.6 mmol) in CH₂Cl₂ (15 mL) was added SOCl₂ (1.35 g, 11.4 mmol). After stirred for 10 min, the reaction mixture was concentrated and dissolved in CH₂Cl₂ (100 mL), TBAF (1.0 M, 38 mL, 38 mmol) was added followed by TMSCN (3.7 g, 38 mmol). After the addition, the reaction mixture was stirred overnight. The reaction mixture was concentrated to give the residue, treated with H₂O and extracted with EtOAc (100 mL×2). The combined organic layers were washed with brine, dried over Na₂SO₄, concentrated. The resulting residue was purified by flash column chromatography to give the titled compound (1.5 g, 45%) as a white solid. MS: M/e 444 (M+1)⁺.

### Step H: 2-(3-ethyl-6-((2S,5R)-5-ethyl-2-methylpiperazin-1-yl)-9-methyl-2-oxo-3,9-dihydro-2H-purin-8-yl)acetonitrile

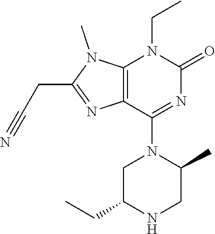 [SMILES: CC[C@@H]1CN(c2nc(=O)n(CC)c3c2nc(CC#N)n3C)[C@@H](C)CN1]

To a stirred solution of tert-butyl (2R,5S)-4-(8-(cyanomethyl)-3-ethyl-9-methyl-2-oxo-3,9-dihydro-2H-purin-6-yl)-2-ethyl-5-methylpiperazine-1-carboxylate (0.4 g, 0.9 mmol) in CH₂Cl₂ (10 mL) was added TFA (2 mL). After then, the reaction mixture was stirred for 2 hours. The reaction mixture was concentrated to give the residue, which was basified to pH=9˜10 with K₂CO₃ (water 5 mL) and extracted with CH₂Cl₂/IPA (3/1, 100 mL×3). The combined organic layers were dried over Na₂SO₄, concentrated, further lyophilized to give the titled compound (0.38 g, crude), which was directly used to the next step. MS: M/e 342 (M+1)⁺.

### Step I: 2-(6-((2S,5R)-4-(1-(3,3-dimethyl-2,3-dihydrobenzo[b][1,4]dioxin-6-yl)ethyl)-5-ethyl-2-methylpiperazin-1-yl)-3-ethyl-9-methyl-2-oxo-3,9-dihydro-2H-purin-8-yl)acetonitrile

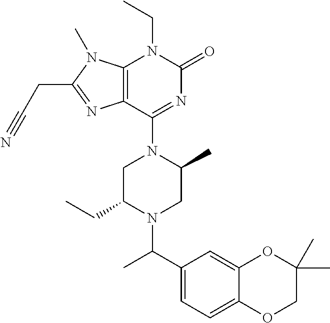 [SMILES: CC[C@@H]1CN(c2nc(=O)n(CC)c3c2nc(CC#N)n3C)[C@@H](C)CN1C(C)c1ccc2c(c1)OC(C)(C)CO2]

A mixture of 2-(3-ethyl-6-((2S,5R)-5-ethyl-2-methylpiperazin-1-yl)-9-methyl-2-oxo-3,9-dihydro-2H-purin-8-yl)acetonitrile (0.38 g, 1.1 mmol), 1-(3,3-dimethyl-2,3-dihydrobenzo[b][1,4]dioxin-6-yl)ethan-1-ol (0.7 g, 3.3 mmol), (cyanomethyl)trimethylphosphonium iodide (1.1 g, 4.43 mmol) and DIPEA (1.43 g, 11 mmol) in CH₃CN (8 mL) was stirred overnight at 105° C. in a sealed tube. The reaction mixture was concentrated to give the residue, treated with H₂O and extracted with EtOAc (30 mL×2). The combined organic layers were washed with brine, dried over Na₂SO₄, concentrated. The reaction solvent was removed under reduced pressure to give the titled Compound A66, which was further separated into Compound A66a (25 mg) and Compound A66b (10 mg) by Prep-HPLC (Method A).

Compound A66a (the earlier peak): 1H NMR (400 MHz, DMSO-d6) δ 6.87-6.73 (m, 3H), 6.21-4.93 (m, 2H), 4.28 (q, J=7.0 Hz, 2H), 4.23-4.18 (m, 1H), 3.88 (d, J=3.9 Hz, 5H), 3.68-3.38 (m, 2H), 3.13 (s, 2H), 2.70-2.45 (m, 1H), 2.35 (s, 1H), 1.49 (d, J=7.5 Hz, 2H), 1.41-1.15 (m, 15H), 1.01 (t, J=7.3 Hz, 3H) ppm. MS: M/e 534 (M+1)⁺.

Compound A66b (the later peak): 1H NMR (400 MHz, DMSO-d6) δ 6.90-6.70 (m, 3H), 6.10-5.05 (m, 2H), 4.28 (q, J=7.0 Hz, 2H), 4.21 (s, 1H), 3.89 (d, J=5.8 Hz, 5H), 3.75-3.35 (m, 2H), 3.12-2.61 (m, 3H), 2.52 (s, 1H), 1.53-1.22 (m, 17H), 0.74 (t, J=7.2 Hz, 3H) ppm. MS: M/e 534 (M+1)⁺.

### Compound A67: 2-(6-((2S,5R)-4-(1-(3,3-dimethyl-2,3-dihydrobenzo[b][1,4]dioxin-6-yl)ethyl)-2,5-diethylpiperazin-1-yl)-9-ethyl-3-methyl-2-oxo-3,9-dihydro-2H-purin-8-yl)acetonitrile

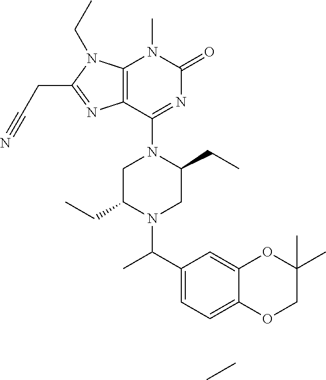

A solution of 2-(6-((2S,5R)-2,5-diethylpiperazin-1-yl)-9-ethyl-3-methyl-2-oxo-3,9-dihydro-2H-purin-8-yl)acetonitrile (50 mg, 0.14 mmol), 1-(3,3-dimethyl-2,3-dihydrobenzo[b][1,4]dioxin-6-yl)ethan-1-ol (29 mg, 0.14 mmol), (cyanomethyl)trimethylphosphonium iodide (102 mg, 0.42 mmol) and DIPEA (90 mg, 0.70 mmol) in CH₃CN (3 ml) was stirred at 100° C. overnight. After completed, the solution was concentrated under reduced pressure to dryness. The resulting residue was purified by Prep-HPLC (Method A) to give the titled compound (42 mg, 58%). ¹H NMR (400 MHz, CD₃OD) δ 7.01-6.75 (m, 3H), 6.42-4.93 (m, 2H), 4.41-4.20 (m, 4H), 3.91 (d, J=6.7 Hz, 2H), 3.77 (s, 3H), 3.48 (s, 2H), 2.89-2.49 (m, 2H), 2.14-1.67 (m, 3H), 1.66-1.36 (m, 8H), 1.32 (d, J=7.3 Hz, 6H), 1.09-0.93 (m, 3H), 0.85-0.60 (m, 3H) ppm. MS: M/e 548 (M+1)⁺

### Compound A68: 2-(6-((2S,5R)-4-(1-(8-fluoro-3,3-dimethyl-2,3-dihydrobenzo[b][1,4]dioxin-6-yl)ethyl)-2,5-dimethylpiperazin-1-yl)-3,9-dimethyl-2-oxo-3,9-dihydro-2H-purin-8-yl)acetonitrile

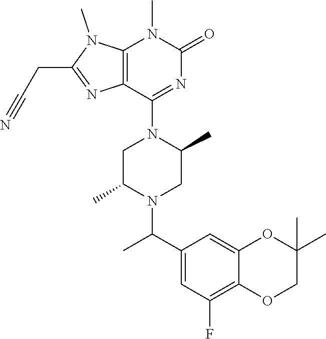 [SMILES: CC(c1cc(F)c2c(c1)OC(C)(C)CO2)N1C[C@H](C)N(c2nc(=O)n(C)c3c2nc(CC#N)n3C)C[C@H]1C]

### Step A: 3,4-difluoro-5-hydroxybenzonitrile

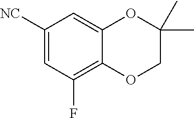

A mixture of 5-bromo-2,3-difluorophenol (3 g, 14 mmol), tBuXPhos Pd G3 (222 mg, 0.28 mmol) and Zn(CN)₂ (3.27 g, 28 mmol) in THE (15 mL) and H₂O (15 mL) was stirred at 40° C. for overnight. The reaction mixture was extracted with EtOAc (50 mL×2). The combined organics were concentrated to dryness. The resulting residue was purified by flash column chromatography to give the titled compound (1.7 g, 76%). MS: M/e 156 (M+1)⁺.

### Step B: 8-fluoro-3,3-dimethyl-2,3-dihydrobenzo[b][1,4]dioxine-6-carbonitrile

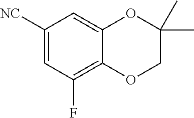

To a solution of 3,4-difluoro-5-hydroxybenzonitrile (1.7 g, 10.9 mmol) and Cs₂CO₃ (10.7 g, 32.9 mmol) in DMAc (50 mL) was added 2,2-dimethyloxirane (6.3 g, 87.2 mol) at room temperature. The reaction mixture was stirred at 100° C. for overnight. The reaction mixture was filtered. H₂O (50 mL) was added to the filtrates. The mixture was extracted with PE (100 mL×3). The organic phases were concentrated and purified by silica gel chromatography (PE:EtOAc=10:1) to give the titled compound (1.3 g, 57%). MS: M/e 208 (M+1)⁺.

### Step C: 8-fluoro-3,3-dimethyl-2,3-dihydrobenzo[b][1,4]dioxine-6-carbaldehyde

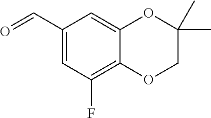

To a solution of 8-fluoro-3,3-dimethyl-2,3-dihydrobenzo[b][1,4]dioxine-6-carbonitrile (1.3 g, 6.2 mmol) in THE (20 mL) was added Dibal-H (9 mL, 9 mmol, 1M in n-Hexane) slowly at 0° C. The reaction mixture was stirred at 0° C. for 2 hours. The reaction was quenched by 2 N HCl (30 mL). The resulting mixture was stirred at room temperature for 1 hours. The mixture was extracted with EtOAc (50 mL×3). The organic phases were concentrated and purified by combi flash to give the titled compound (0.9 g, 69%). MS: M/e 211 (M+1)⁺.

### Step D: 1-(8-fluoro-3,3-dimethyl-2,3-dihydrobenzo[b][1,4]dioxin-6-yl)ethan-1-ol

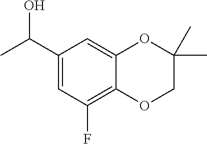 [SMILES: CC(O)c1cc(F)c2c(c1)OC(C)(C)CO2]

To a solution of 8-fluoro-3,3-dimethyl-2,3-dihydrobenzo[b][1,4]dioxine-6-carbaldehyde (0.9 g, 4.3 mmol) in THE (10 mL) was added MeMgBr (2.7 mL, 8.6 mmol, 3M in Et₂O) slowly at 0° C. The resulting mixture was stirred at 0° C. for 2 hours. The reaction mixture was quenched with H₂O (10 mL) and extracted with EtOAc (30 mL×3). The combined organic phases were concentrated and purified by combi flash to give the titled compound (550 mg, 57% yield). MS: M/e 227 (M+1)⁺.

### Step E: 2-(6-((2S,5R)-4-(1-(8-fluoro-3,3-dimethyl-2,3-dihydrobenzo[b][1,4]dioxin-6-yl)ethyl)-2,5-dimethylpiperazin-1-yl)-3,9-dimethyl-2-oxo-3,9-dihydro-2H-purin-8-yl)acetonitrile

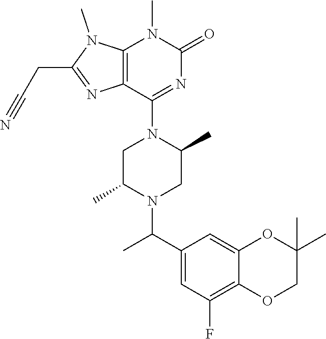 [SMILES: CC(c1cc(F)c2c(c1)OC(C)(C)CO2)N1C[C@H](C)N(c2nc(=O)n(C)c3c2nc(CC#N)n3C)C[C@H]1C]

To a solution of 1-(8-fluoro-3,3-dimethyl-2,3-dihydrobenzo[b][1,4]dioxin-6-yl)ethan-1-ol

(64 mg, 0.28 mmol), 2-(6-((2S,5R)-2,5-dimethylpiperazin-1-yl)-3,9-dimethyl-2-oxo-3,9-dihydro-2H-purin-8-yl)acetonitrile (60 mg, 0.19 mmol) and (cyanomethyl) trimethyl phosphonium iodide (68 mg, 0.28 mmol) in CH₃CN (5 mL) was added DIPEA (116 mg, 0.9 mmol). The mixture was sealed in a bottle and heated at 100° C. for 16 hours. The mixture was cooled to room temperature, diluted with water, extracted with EtOAc (10 mL×2), washed with brine, dried over Na₂SO₄, filtered, and concentrated to dryness. The resulting residue was purified by Prep-TLC (DCM:MeOH=15:1) to give the titled Compound A68 (23 mg, 23%), which was separated into Compound A68a (5 mg) and Compound A68b (4 mg) by chiral Prep-HPLC. The chiral separation conditions are shown below.

| | |
|---|---|
| Column | CHIRALPAK IH |
| Column Size | 2 cm × 25 cm, 5 um |
| Mobile Phase A | MtBE (0.5% 2 mM NH3—MeOH) |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A68: ¹H NMR (400 MHz, CD₃OD) δ 6.73-6.65 (m, 2H), 6.01-5.95 (m, 1H), 5.50-5.03 (m, 1H), 4.21 (s, 2H), 3.95-3.91 (m, 4H), 3.74-3.71 (m, 4H), 3.29-3.25 (m, 2H), 2.83-2.12 (m, 3H), 1.44-1.26 (m, 12H), 1.05-0.90 (m, 3H) ppm. MS: M/e 524 (M+1)⁺.

Compound A68a (the earlier peak) ¹H NMR (400 MHz, CD₃OD) δ 6.71 (d, J=11.3 Hz, 1H), 6.64 (s, 1H), 6.19-5.95 (m, 1H), 5.50-5.25 (m, 1H), 4.20 (s, 1H), 3.93 (d, J=3.4 Hz, 4H), 3.78 (s, 3H), 3.50-3.10 (m, 2H), 2.90-2.75 (m, 5H), 1.46-1.35 (m, 6H), 1.29-1.24 (m, 5H), 0.89-0.88 (m, 3H) ppm. MS: M/e 524 (M+1)⁺.

Compound A68b (the later peak) ¹H NMR (400 MHz, CD₃OD) δ 6.71 (d, J=11.3 Hz, 1H), 6.63 (s, 1H), 6.01-5.75 (m, 1H), 5.23-5.03 (m, 1H), 4.21 (s, 1H), 3.95-3.93 (m, 5H), 3.78 (s, 3H), 3.51-3.47 (m, 1H), 2.83-2.51 (m, 4H), 2.25-2.22 (m, 1H), 1.40-1.26 (m, 12H), 1.05-0.95 (m, 3H) ppm. MS: M/e 524 (M+1)⁺.

### Compound A69: 2-(6-((2S,5R)-4-(1-(7-fluoro-3,3-dimethyl-2,3-dihydrobenzo[b][1,4]dioxin-6-yl)ethyl)-2,5-dimethylpiperazin-1-yl)-3,9-dimethyl-2-oxo-3,4,5,9-tetrahydro-2H-purin-8-yl)acetonitrile

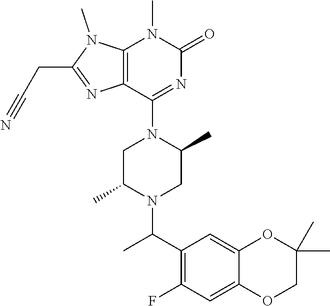 [SMILES: CC(c1cc2c(cc1F)OCC(C)(C)O2)N1C[C@H](C)N(c2nc(=O)n(C)c3c2nc(CC#N)n3C)C[C@H]1C]

### Step A: 2,4-difluoro-5-hydroxybenzonitrile

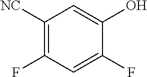 [SMILES: N#Cc1cc(O)c(F)cc1F]

To a solution of 5-bromo-2,4-difluorophenol (1.0 g, 4.785 mmol), t-BuXphos Pd G3 (190 mg, 0.239 mmol), t-BuXphos (204 mg, 0.48 mmol) and Zn(CN)₂ (1.12 g, 9.570 mmol) in THE (20 mL) and H₂O (10 mL) was degassed 3 times under N₂ atmosphere. Then the mixture was stirred at 70° C. for 12 hours. After filtration, the reaction mixture was diluted with water and extracted with EtOAc (50 mL×2). The combined organic layers were washed with brine, dried over Na₂SO₄, filtered and concentrated to dryness. The resulting residue was purified by flash column chromatography (PE:EtOAc=5:1) to give the titled compound (730 mg, 98%).

### Step B: 7-fluoro-3,3-dimethyl-2,3-dihydrobenzo[b][1,4]dioxine-6-carbonitrile

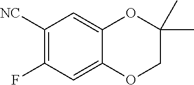

To a solution of 2,4-difluoro-5-hydroxybenzonitrile (700 mg, 4.516 mmol) and Cs₂CO₃ (5.15 g, 13.548 mmol) in DMAc (25 mL) was added 2,2-dimethyloxirane (4.885 g, 67.74 mmol). The reaction was stirred at 90° C. for 12 hours. After filtration, the reaction mixture was diluted with water, extracted with PE (60 mL×3), washed with brine, dried over Na₂SO₄, filtered, and concentrated to dryness. The resulting residue was purified by flash column chromatography (PE:EtOAc=5:1) to give the titled compound (220 mg, 24%). ¹H NMR (400 MHz, CDCl₃) δ 7.09 (d, J=6.1 Hz, 1H), 6.66 (d, J=9.7 Hz, 1H), 3.88 (s, 2H), 1.36 (s, 6H) ppm.

### Step C: 7-fluoro-3,3-dimethyl-2,3-dihydrobenzo[b][1,4]dioxine-6-carbaldehyde

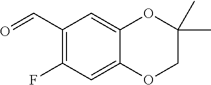

To a solution of 7-fluoro-3,3-dimethyl-2,3-dihydrobenzo[b][1,4]dioxine-6-carbaldehyde (200 mg, 0.966 mmol) in THE (15 mL) was degassed 3 times under N₂ atmosphere. Then added DIBAL-H (1.45 mL, 1M, 1.45 mmol) dropwise at 0° C. The mixture was stirred at 0° C. for 4 hours. The reaction solution was quenched by 1M HCl. After filtration, the reaction mixture was diluted with water, extracted with EtOAc (50 mL×3), washed with brine, dried over Na₂SO₄, filtered, and concentrated to dryness. The resulting residue was purified by flash column chromatography (PE:EtOAc=5:1) to give the titled compound (120 mg, 54%).

### Step D: 1-(7-fluoro-3,3-dimethyl-2,3-dihydrobenzo[b][1,4]dioxin-6-yl)ethan-1-ol

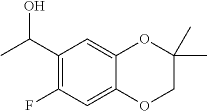 [SMILES: CC(O)c1cc2c(cc1F)OCC(C)(C)O2]

To a solution of 7-fluoro-3,3-dimethyl-2,3-dihydrobenzo[b][1,4]dioxine-6-carbaldehyde (110 mg, 0.523 mmol) in THE (10 mL) was degassed 3 times under N₂ atmosphere. Then added CH₃MgBr (0.35 mL, 3M, 1.048 mmol) dropwise at 0° C. The mixture was stirred at 0° C. for 4 hours. The reaction solution was quenched with saturated NH₄Cl, extracted with EtOAc (50 mL×2), washed with brine, dried over Na₂SO₄, filtered, and concentrated to dryness. The resulting residue was purified by flash column chromatography (PE:EtOAc=5:1) to give the titled compound (86 mg, 73%).

### Step E: 2-(6-((2S,5R)-4-(1-(7-fluoro-3,3-dimethyl-2,3-dihydrobenzo[b][1,4]dioxin-6-yl)ethyl)-2,5-dimethylpiperazin-1-yl)-3,9-dimethyl-2-oxo-3,4,5,9-tetrahydro-2H-purin-8-yl)acetonitrile

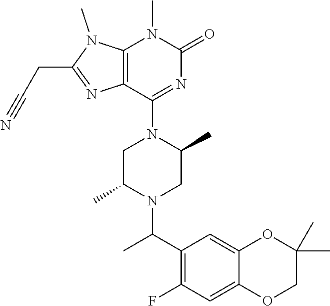 [SMILES: CC(c1cc2c(cc1F)OCC(C)(C)O2)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (80 mg, 0.254 mmol), 1-(7-fluoro-3,3-dimethyl-2,3-dihydrobenzo[b][1,4]dioxin-6-yl)ethan-1-ol (86 mg, 0.381 mmol), (cyanomethyl)trimethylphosphonium iodide (123 mg. 0.508 mmol) and DIPEA (98 mg, 0.762 mol) in CH₃CN (1 ml). The mixture solution was degassed 3 times under N₂ atmosphere. Then the mixture solution was stirred at 100° C. for 24 hours. The reaction mixture was concentrated under reduced pressure. The resulting residue was purified by Prep-TLC (DCM:MeOH=15:1) to give the titled Compound A69 (29 mg), which was further separated into Compound A69a (8 mg) and Compound A69b (6 mg) by Prep-HPLC (Method A).

Compound A69: ¹H NMR (400 MHz, CD₃OD) δ 7.08-6.90 (m, 1H), 6.54 (dt, J=24.0, 11.8 Hz, 1H), 6.15-5.52 (m, 1H), 5.37-4.96 (m, 0.5H), 4.72-4.49 (m, 0.5H), 4.21 (s, 1H), 3.93 (s, 3H), 3.88 (d, J=6.0 Hz, 2H), 3.80 (s, 3H), 3.76-3.67 (m, 1.5H), 3.58-3.47 (m, 0.5H), 3.23 (dd, J=14.8, 7.4 Hz, 1H), 2.95-2.87 (m, 1H), 2.80-2.55 (m, 1.5H), 2.32-2.19 (m, 0.5H), 1.37 (d, J=5.2 Hz, 3H), 1.32 (d, J=5.9 Hz, 6H), 1.29 (s, 3H), 1.02 (d, J=5.4 Hz, 1.5H), 0.91 (d, J=5.6 Hz, 1.5H) ppm. MS: M/e 524 (M+1)⁺

Compound A69a (the earlier peak): ¹H NMR (400 MHz, CD₃OD) δ 6.95 (d, J=6.9 Hz, 1H), 6.56 (d, J=10.7 Hz, 1H), 6.20-6.00 (m, 0.5H), 5.78-5.46 (m, 0.5H), 5.26-5.05 (m, 0.5H), 4.76-4.51 (m, 0.5H), 4.21 (s, 1H), 3.93 (s, 3H), 3.90 (d, J=4.4 Hz, 2H), 3.78 (s, 3H), 3.58-3.47 (m, 1H), 2.95-2.87 (m, 2H), 2.83-2.65 (m, 2H), 2.32-1.98 (m, 1H), 1.42 (d, J=4.4 Hz, 3H), 1.32 (d, J=4.0 Hz, 6H), 1.28 (d, J=6.3 Hz, 3H), 0.90 (d, J=5.9 Hz, 3H) ppm. MS: M/e 524 (M+1)⁺

Compound A69b (the later peak): ¹H NMR (400 MHz, CD₃OD) δ 7.05 (d, J=6.9 Hz, 1H), 6.49 (d, J=11.0 Hz, 1H), 6.02-5.78 (m, 1H), 5.14-5.01 (m, 0.5H), 4.84-4.71 (m, 0.5H), 4.21 (s, 1H), 3.93 (s, 3H), 3.88 (s, 2H), 3.78 (s, 3H), 3.54-3.31 (m, 2H), 2.74-2.53 (m, 2H), 2.34-1.98 (m, 2H), 1.31 (s, 6H), 1.30 (s, 6H), 1.02 (d, J=6.2 Hz, 3H) ppm. MS: M/e 524 (M+1)⁺

### Compound A70: 2-(6-((2S,5R)-4-(1-(5-fluoro-3,3-dimethyl-2,3-dihydrobenzo[b][1,4]dioxin-6-yl)ethyl)-2,5-dimethylpiperazin-1-yl)-3,9-dimethyl-2-oxo-3,9-dihydro-2H-purin-8-yl)acetonitrile

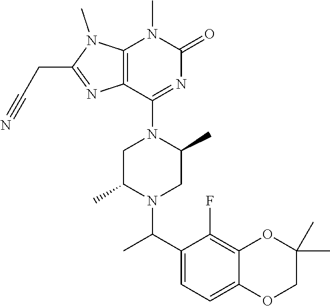 [SMILES: CC(c1ccc2c(c1F)OC(C)(C)CO2)N1C[C@H](C)N(c2nc(=O)n(C)c3c2nc(CC#N)n3C)C[C@H]1C]

### Step A: 2,4-difluoro-3-hydroxybenzonitrile

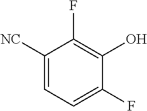 [SMILES: N#Cc1ccc(F)c(O)c1F]

To a solution of 3-bromo-2,6-difluorophenol (1.0 g, 4.785 mmol), t-BuXphos Pd G3 (190 mg, 0.239 mmol), t-BuXphos (204 mg, 0.48 mmol) and Zn(CN)₂ (1.12 g, 9.570 mmol) in THE (20 mL) and H₂O (10 mL) was degassed 3 times under N₂ atmosphere. Then the mixture was stirred at 70° C. for 12 hours. After filtration, the reaction mixture was diluted with water and extracted with EtOAc (50 mL×2). The combined organic layers were washed with brine, dried over Na₂SO₄, filtered and concentrated to dryness. The resulting residue was purified by flash column chromatography (PE:EtOAc=5:1) to give the titled compound (690 mg, 93%). MS: M/e 156 (M+1)⁺

### Step B: 5-fluoro-3,3-dimethyl-2,3-dihydrobenzo[b][1,4]dioxine-6-carbonitrile

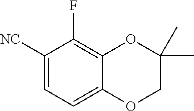

To a solution of 2,4-difluoro-3-hydroxybenzonitrile (690 mg, 4.452 mmol) and Cs₂CO₃ (5.08 g, 13.355 mmol) in DMAc (25 mL) was added 2,2-dimethyloxirane (4.815 g, 66.78 mmol). The reaction was stirred at 90° C. for 12 hours. After filtration, the reaction mixture was diluted with water, extracted with PE (60 mL×3), washed with brine, dried over Na₂SO₄, filtered, and concentrated to dryness. The resulting residue was purified by flash column chromatography (PE:EtOAc=5:1) to give the titled compound (500 mg, 54%). MS: M/e 208 (M+1)⁺

### Step C: 5-fluoro-3,3-dimethyl-2,3-dihydrobenzo[b][1,4]dioxine-6-carbaldehyde

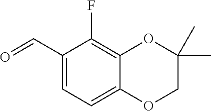

To a solution of 5-fluoro-3,3-dimethyl-2,3-dihydrobenzo[b][1,4]dioxine-6-carbonitrile (500 mg, 2.415 mmol) in THE (50 mL) was degassed 3 times under N₂ atmosphere. Then added DIBAL-H (4.8 mL, 1M, 4.831 mmol) dropwise at 0° C. The mixture was stirred at 0° C. for 4 hours. The reaction solution was quenched by 1M HCl. After filtration, the reaction mixture was diluted with water, extracted with EtOAc (50 mL×3), washed with brine, dried over Na₂SO₄, filtered, and concentrated to dryness. The resulting residue was purified by flash column chromatography (PE:EtOAc=5:1) to give the titled compound (400 mg, 79%). MS: M/e 211 (M+1)⁺

### Step D: 1-(5-fluoro-3,3-dimethyl-2,3-dihydrobenzo[b][1,4]dioxin-6-yl)ethan-1-ol

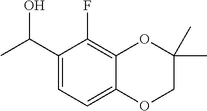 [SMILES: CC(O)c1ccc2c(c1F)OC(C)(C)CO2]

To a solution of 5-fluoro-3,3-dimethyl-2,3-dihydrobenzo[b][1,4]dioxine-6-carbaldehyde (400 mg, 1.905 mmol) in THE (20 mL) was degassed 3 times under N₂ atmosphere. Then added CH₃MgBr (1.3 mL, 3M, 3.810 mmol) dropwise at 0° C. The mixture was stirred at 0° C. for 4 hours. The reaction solution was quenched with saturated NH₄Cl, extracted with EtOAc (50 mL×2), washed with brine, dried over Na₂SO₄, filtered, and concentrated to dryness. The resulting residue was purified by flash column chromatography (PE:EtOAc=5:1) to give the titled compound (320 mg, 74%). ¹H NMR (400 MHz, CDCl₃) δ 6.84 (dd, J=13.2, 6.3 Hz, 1H), 6.68 (dd, J=20.2, 9.8 Hz, 1H), 5.04 (dd, J=23.0, 6.2 Hz, 1H), 3.95 (s, 2H), 1.49 (dd, J=8.5, 3.5 Hz, 3H), 1.40 (s, 6H) ppm.

### Step E: 2-(6-((2S,5R)-4-(1-(5-fluoro-3,3-dimethyl-2,3-dihydrobenzo[b][1,4]dioxin-6-yl)ethyl)-2,5-dimethylpiperazin-1-yl)-3,9-dimethyl-2-oxo-3,9-dihydro-2H-purin-8-yl)acetonitrile

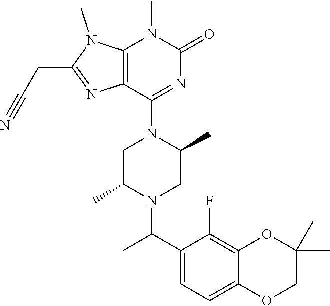 [SMILES: CC(c1ccc2c(c1F)OC(C)(C)CO2)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (80 mg, 0.254 mmol), 1-(5-fluoro-3,3-dimethyl-2,3-dihydrobenzo[b][1,4]dioxin-6-yl)ethan-1-ol (86 mg, 0.381 mmol), (cyanomethyl)trimethylphosphonium iodide (123 mg. 0.508 mmol) and DIPEA (98 mg, 0.762 mol) in CH₃CN (1 ml). The mixture solution was degassed 3 times under N₂ atmosphere. Then the mixture solution was stirred at 100° C. for 24 hours. The reaction mixture was concentrated under reduced pressure. The resulting residue was purified by Prep-TLC (DCM:MeOH=15:1) to give the titled compound (38 mg, 29%). ¹H NMR (400 MHz, CD₃OD) δ 7.16-6.89 (m, 1H), 6.78-6.56 (m, 1H), 6.04-5.45 (m, 1H), 5.35-5.08 (m, 0.5H), 4.73-4.54 (m, 0.5H), 4.27-4.08 (m, 1.5H), 4.01-3.96 (m, 2.5H), 3.86 (s, 3H), 3.79 (s, 3H), 3.76-3.66 (m, 0.5H), 3.57-3.36 (m, 1H), 3.25-3.21 (m, 0.5H), 2.99-2.52 (m, 2.5H), 2.26 (t, J=11.3 Hz, 0.5H), 1.39 (d, J=8.0 Hz, 2H), 1.34 (d, J=6.2 Hz, 6H), 1.27 (d, J=8.5 Hz, 4H), 1.06-0.85 (m, 3H) ppm. MS: M/e 524 (M+1)⁺

### Compound A71: 2-(9-ethyl-6-((2S,5R)-4-(1-(5-fluoro-3,3-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile

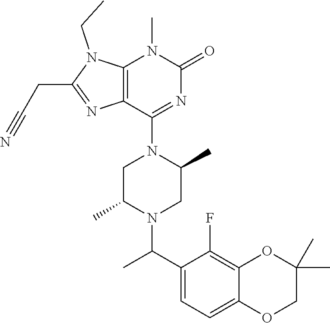 [SMILES: CCn1c(CC#N)nc2c(N3C[C@@H](C)N(C(C)c4ccc5c(c4F)OC(C)(C)CO5)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (80 mg, 0.243 mmol), 1-(5-fluoro-3,3-dimethyl-2,3-dihydrobenzo[b][1,4]dioxin-6-yl)ethan-1-ol (82 mg, 0.365 mmol), (cyanomethyl)trimethylphosphonium iodide (119 mg. 0.486 mmol) and DIPEA (94 mg, 0.729 mol) in CH₃CN (1 ml). The mixture solution was degassed 3 times under N₂ atmosphere. Then the mixture solution was stirred at 100° C. for 24 hours. The reaction mixture was concentrated under reduced pressure. The resulting residue was purified by Prep-TLC (DCM:MeOH=15:1) to give the titled compound (46 mg, 35%). ¹H NMR (400 MHz, CD₃OD) δ 7.14-6.95 (m, 1H), 6.67 (q, J=9.9 Hz, 1H), 6.17-5.39 (m, 1H), 5.35-4.94 (m, 0.5H), 4.65-4.58 (m, 0.5H), 4.45-4.29 (m, 2H), 4.21-4.06 (m, 1H), 3.92 (d, J=11.1 Hz, 2H), 3.76 (s, 3H), 3.58-3.38 (m, 1H), 3.30-3.05 (m, 2H), 2.93-2.55 (m, 2H), 2.35-1.95 (m, 1H), 1.46 (t, J=7.1 Hz, 3H), 1.37-1.32 (m, 6H), 1.26 (d, J=17.2 Hz, 6H), 1.03 (d, J=4.7 Hz, 1.5H), 0.92 (d, J=5.6 Hz, 1.5H) ppm. MS: M/e 538 (M+1)⁺

### Compound A72: 2-(6-((2S,5R)-4-(1-(8-chloro-3,3-dimethyl-2,3-dihydrobenzo[b][1,4]dioxin-6-yl)ethyl)-2,5-dimethylpiperazin-1-yl)-9-ethyl-3-methyl-2-oxo-3,9-dihydro-2H-purin-8-yl)acetonitrile

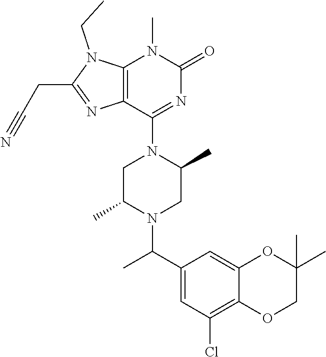 [SMILES: CCn1c(CC#N)nc2c(N3C[C@@H](C)N(C(C)c4cc(Cl)c5c(c4)OC(C)(C)CO5)C[C@@H]3C)nc(=O)n(C)c21]

### Step A: 3-chloro-4-fluoro-5-hydroxybenzonitrile

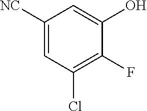 [SMILES: N#Cc1cc(O)c(F)c(Cl)c1]

A mixture of 5-bromo-3-chloro-2-fluorophenol (1 g, 4.4 mmol), tBuXPhos Pd G3 (71 mg, 0.09 mmol) and Zn(CN)₂ (1.0 g, 8.8 mmol) in THE (5 mL) and H₂O (5 mL) was stirred at 40° C. for overnight. The reaction mixture was extracted with EtOAc (20 mL×2). The combined organics were concentrated to dryness. The resulting residue was purified by flash column chromatography to give the titled compound (210 mg, 28%). MS: M/e 172 (M+1)⁺.

### Step B: 8-chloro-3,3-dimethyl-2,3-dihydrobenzo[b][1,4]dioxine-6-carbonitrile

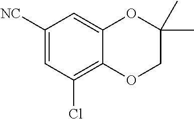

To a solution of 3-chloro-4-fluoro-5-hydroxybenzonitrile (210 mg, 1.2 mmol) and Cs₂CO₃ (1.2 g, 3.6 mmol) in DMAc (5 mL) was added 2,2-dimethyloxirane (690 mg, 9.6 mol) at room temperature. The reaction mixture was stirred at 100° C. for overnight. The reaction mixture was filtered. H₂O (10 mL) was added to the filtrates. The mixture was extracted with PE (50 mL×3). The organic phases were concentrated to dryness. The resulting residue was purified by flash column chromatography (PE:EtOAc=10:1) to give the titled compound (170 mg, 62%). MS: M/e 224 (M+1)⁺.

### Step C: 1-(8-chloro-3,3-dimethyl-2,3-dihydrobenzo[b][1,4]dioxin-6-yl)ethan-1-one

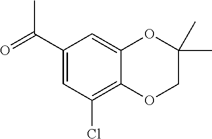 [SMILES: CC(=O)c1cc(Cl)c2c(c1)OC(C)(C)CO2]

To a solution of 8-chloro-3,3-dimethyl-2,3-dihydrobenzo[b][1,4]dioxine-6-carbonitrile (170 mg, 0.76 mmol) in THE (2 mL) was added MeMgBr (1.5 mL, 1.5 mmol, 1M in THF) slowly at 0° C. The reaction mixture was stirred at 70° C. for overnight. The reaction was quenched by 2 N HCl (10 mL). The resulting mixture was stirred at room temperature for 1 hours. The mixture was extracted with EtOAc (50 mL×3). The organic phases were concentrated to dryness. The resulting residue was purified by flash column chromatography to give the titled compound (150 mg, 82%). MS: M/e 241 (M+1)⁺.

### Step D: 1-(8-chloro-3,3-dimethyl-2,3-dihydrobenzo[b][1,4]dioxin-6-yl)ethan-1-ol

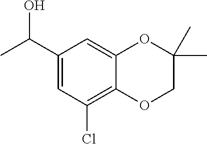 [SMILES: CC(O)c1cc(Cl)c2c(c1)OC(C)(C)CO2]

To a solution of 1-(8-chloro-3,3-dimethyl-2,3-dihydrobenzo[b][1,4]dioxin-6-yl)ethan-1-one (150 mg, 0.63 mmol) in MeOH (5 mL) was added NaBH₄ (26 mg, 0.7 mmol) slowly at 0° C. The resulting mixture was stirred at room temperature for 2 hours. The reaction mixture was quenched with H₂O (10 mL) and extracted with EtOAc (30 mL×3). The combined organic phases were concentrated to dryness. The resulting residue was purified by flash column chromatography to give the titled compound (120 mg, 80% yield). MS: M/e 243 (M+1)⁺.

### Step E: 2-(6-((2S,5R)-4-(1-(8-chloro-3,3-dimethyl-2,3-dihydrobenzo[b][1,4]dioxin-6-yl)ethyl)-2,5-dimethylpiperazin-1-yl)-9-ethyl-3-methyl-2-oxo-3,9-dihydro-2H-purin-8-yl)acetonitrile

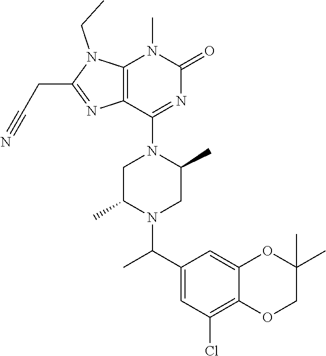 [SMILES: CCn1c(CC#N)nc2c(N3C[C@@H](C)N(C(C)c4cc(Cl)c5c(c4)OC(C)(C)CO5)C[C@@H]3C)nc(=O)n(C)c21]

To a solution of 1-(8-chloro-3,3-dimethyl-2,3-dihydrobenzo[b][1,4]dioxin-6-yl)ethan-1-ol (60 mg, 0.28 mmol), 2-(6-((2S,5R)-2,5-dimethylpiperazin-1-yl)-9-ethyl-3-methyl-2-oxo-3,9-dihydro-2H-purin-8-yl)acetonitrile (60 mg, 0.19 mmol) and (cyanomethyl) trimethyl phosphonium iodide (68 mg, 0.28 mmol) in CH₃CN (5 mL) was added DIPEA (116 mg, 0.9 mmol). The mixture was sealed in a bottle and heated at 100° C. for 16 hours. The mixture was cooled to room temperature, diluted with water, extracted with EtOAc (10 mL×2), washed with brine, dried over Na₂SO₄, filtered, and concentrated to dryness. The resulting residue was purified by Prep-TLC (DCM:MeOH=15:1) to give the titled compound (18 mg, 18%). ¹H NMR (400 MHz, CD₃OD) δ 6.96 (s, 1H), 6.79 (s, 1H), 6.01-5.75 (m, 1H), 5.28-5.01 (m, 1H), 4.38-4.36 (m, 2H), 4.23 (s, 2H), 3.99 (s, 2H) 3.76 (s, 3H), 3.75-3.30 (m, 2H), 2.90-2.75 (m, 3H), 1.49-1.44 (m, 3H), 1.34-1.24 (m, 12H), 1.05-0.90 (m, 3H) ppm. MS: M/e 554 (M+1)⁺.

### Compound A74: 2-(6-((2S,5R)-4-(1-(3,3-dimethyl-2,3-dihydro-[1,4]dioxino[2,3-b]pyridin-6-yl)ethyl)-2,5-dimethylpiperazin-1-yl)-9-ethyl-3-methyl-2-oxo-3,9-dihydro-2H-purin-8-yl)acetonitrile

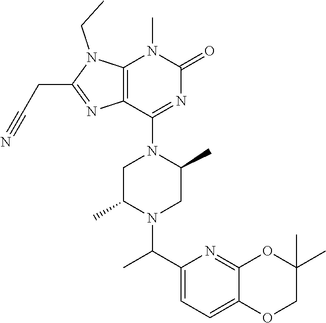 [SMILES: CCn1c(CC#N)nc2c(N3C[C@@H](C)N(C(C)c4ccc5c(n4)OC(C)(C)CO5)C[C@@H]3C)nc(=O)n(C)c21]

### Step A: 2-(6-((2S,5R)-4-(1-(3,3-dimethyl-2,3-dihydro-[1,4]dioxino[2,3-b]pyridin-6-yl)ethyl)-2,5-dimethylpiperazin-1-yl)-9-ethyl-3-methyl-2-oxo-3,9-dihydro-2H-purin-8-yl)acetonitrile

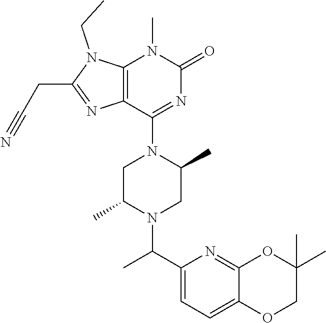 [SMILES: CCn1c(CC#N)nc2c(N3C[C@@H](C)N(C(C)c4ccc5c(n4)OC(C)(C)CO5)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (300 mg, 0.91 mmol), 1-(3,3-dimethyl-2,3-dihydro-[1,4]dioxino[2,3-b]pyridin-6-yl)ethan-1-ol (190 mg, 0.91 mmol), (cyanomethyl)trimethylphosphonium iodide (662 mg, 2.74 mmol) and DIPEA (588 mg, 4.56 mmol) in MeCN (15 ml) was stirred at 100° C. overnight. After completed, the solution was concentrated under reduced pressure, The resulting residue was purified by Prep-HPLC (Method A) to give the titled Compound A74 (121 mg, 26%), which was further purified by Prep-HPLC (Method A) to give the titled Compound A74a (40 mg) and Compound A74b (29 mg).

Compound A74: ¹H NMR (400 MHz, CD₃OD) δ 7.31 (dd, J=8.0, 5.2 Hz, 1H), 7.12 (dd, J=8.1, 2.7 Hz, 1H), 6.50-4.50 (m, 2H), 4.37 (q, J=6.9 Hz, 2H), 4.22 (s, 1H), 3.96 (s, 2H), 3.76 (s, 3H), 3.73-3.52 (m, 2H), 3.50-3.31 (m, 2H), 2.95-2.77 (m, 1.5H), 2.20-2.10 (m, 0.5H), 1.46 (t, J=7.0 Hz, 6H), 1.41 (s, 6H), 1.30 (d, J=6.6 Hz, 3H), 1.11-0.91 (m, 3H) ppm. MS: M/e 521 (M+1)⁺

Compound A74a (the earlier peak): ¹H NMR (400 MHz, CD₃OD) δ 7.32 (d, J=8.1 Hz, 1H), 7.12 (d, J=8.0 Hz, 1H), 6.32-5.45 (m, 1H), 5.39-4.89 (m, 1H), 4.36 (d, J=7.2 Hz, 2H), 4.30-4.20 (m, 1H), 3.96 (s, 2H), 3.76 (s, 3H), 3.64 (d, J=5.6 Hz, 1H), 3.42-3.31 (m, 2H), 2.90 (s, 2H), 2.81 (d, J=11.9 Hz, 1H), 1.46 (t, J=7.1 Hz, 6H), 1.41 (s, 6H), 1.29 (d, J=6.5 Hz, 3H), 0.93 (d, J=6.3 Hz, 3H) ppm. MS: M/e 521 (M+1)⁺

Compound A74b (the later peak): ¹H NMR (400 MHz, CD₃OD) δ 7.30 (d, J=8.0 Hz, 1H), 7.14 (d, J=8.1 Hz, 1H), 6.18-5.63 (m, 1H), 5.27-4.90 (m, 1H), 4.37 (q, J=7.0 Hz, 2H), 3.95 (s, 2H), 3.76 (s, 3H 3.59-3.51 (m, 1H), 3.49-3.39 (m, 1H), 3.30 (s, 3H), 2.72 (s, 1H), 2.18 (d, J=11.8 Hz, 1H), 1.46 (t, J=7.2 Hz, 3H), 1.40 (s, 6H), 1.31 (d, J=6.3 Hz, 6H), 1.02 (d, J=5.2 Hz, 3H) ppm. MS: M/e 521 (M+1)⁺

### Compound A77: 2-(6-((2S,5R)-4-(1-(4,4-difluorochroman-6-yl)ethyl)-2,5-dimethylpiperazin-1-yl)-3,9-dimethyl-2-oxo-3,9-dihydro-2H-purin-8-yl)acetonitrile

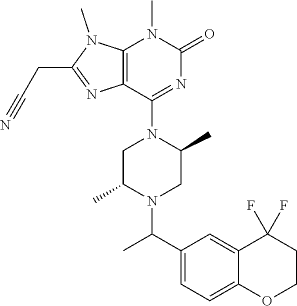 [SMILES: CC(c1ccc2c(c1)C(F)(F)CCO2)N1C[C@H](C)N(c2nc(=O)n(C)c3c2nc(CC#N)n3C)C[C@H]1C]

### Step A: 6-bromospiro[chromane-4,2′-[1,3] dithiolane]

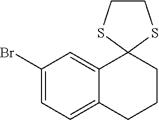 [SMILES: Brc1ccc2c(c1)C1(CCC2)SCCS1]

To a stirred solution of 6-bromo-chroman-4-one (2 g, 8.8 mmol) in DCM (20 mL) at 0° C. are added 1,2-ethanedithiol (1.7 g, 17.6 mmol) and boron trifluoride etherate (640 mg, 4.4 mmol). The reaction mixture is stirred at room temperature for overnight before it is poured into 1.0 N NaOH solution. The mixture is extracted with DCM (50 mL) and the organic layers are separated, concentrated and purified by combi flash to give the titled compound (2.3 g, 96%) MS: M/e 303 (M+1)⁺.

### Step B: 6-bromo-4,4-difluorochromane

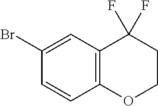 [SMILES: FC1(F)CCOc2ccc(Br)cc21]

A suspension of N-iodosuccinimide (3.3 g, 15 mmol) in DCM (40 mL) is cooled down to −70° C. Hydrogen fluoride pyridine complex (2.5 g, 30 mmol) is added dropwise. A solution of 6-bromospiro[chromane-4,2′-[1,3] dithiolane](2.3 g, 7.6 mmol) in DCM is added dropwise and the mixture is stirred at −70° C. for 30 min. Then the reaction solution is poured into a mixture of hexane (10 mL) and DCM (10 mL) and the resulting mixture is filtered through a short pad of silica gel. The filtrate is concentrated and purified by combi flash to give the title compound (1.3 g, 69%). MS: M/e 249 (M+1)⁺.

### Step C: 1-(4,4-difluorochroman-6-yl)ethan-1-one

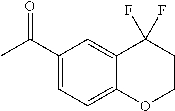

A mixture of 6-bromo-4,4-difluorochromane (500 mg, 2.0 mmol), tributyl(1-ethoxyvinyl) stannane (1.1 g, 3.0 mmol) and Pd(PPh₃)₂Cl₂ (140 mg, 0.2 mmol) in toluene (5 mL) was stirred at 100° C. under N₂ for 4 hours. The reaction mixture was quenched with saturated NaHCO₃ aq. (10 mL), extracted with EtOAc (25 mL×3), combined, washed brine (10 mL×3), dried and concentrated to dryness. The resulting oil was diluted with THE (5 mL). Then to the solution was added HCl (2 mL, 4 M in 1,4-dioxane) in drops and the mixture was stirred at room temperature for 1 hour. The reaction mixture was diluted with EtOAc (20 mL), treated with saturated NaHCO₃ aq. to pH˜8, washed with brine (20 mL×3), dried over Na₂SO₄ and concentrated to dryness. The resulting residue was purified by flash column chromatography to give the titled compound (320 mg, 75%). MS: M/e 213 (M+1)⁺.

### Step D: 1-(4,4-difluorochroman-6-yl)ethan-1-ol

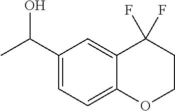

To a solution of 1-(4,4-difluorochroman-6-yl)ethan-1-one (320 mg, 1.5 mmol) in MeOH (10 mL) was added NaBH₄ (66 mg, 1.8 mmol) at room temperature and the resulting mixture was stirred at room temperature for 2 hours. The reaction mixture was concentrated to dryness. The resulting residue was treated with saturated NaHCO₃ aq., extracted with EtOAc (10 mL×3). The combined organic layers were washed with brine (10 mL×3), dried over Na₂SO₄ and concentrated to dryness. The resulting residue was purified by flash column chromatography to give the titled compound (280 mg, 88%). MS: M/e 215 (M+1)⁺.

### Step E: 2-(6-((2S,5R)-4-(1-(4,4-difluorochroman-6-yl)ethyl)-2,5-dimethylpiperazin-1-yl)-3,9-dimethyl-2-oxo-3,9-dihydro-2H-purin-8-yl)acetonitrile

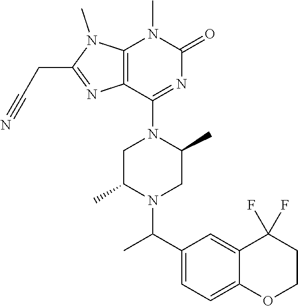 [SMILES: CC(c1ccc2c(c1)C(F)(F)CCO2)N1C[C@H](C)N(c2nc(=O)n(C)c3c2nc(CC#N)n3C)C[C@H]1C]

To a solution of 1-(4,4-difluorochroman-6-yl)ethan-1-ol (60 mg, 0.28 mmol), 2-(6-((2S, 5R)-2,5-dimethylpiperazin-1-yl)-3,9-dimethyl-2-oxo-3,9-dihydro-2H-purin-8-yl) acetonitrile (60 mg, 0.19 mmol) and (cyanomethyl) trimethyl phosphonium iodide (68 mg, 0.28 mmol) in CH₃CN (5 mL) was added DIPEA (116 mg, 0.9 mmol). The mixture was sealed in a bottle and heated at 100° C. for 16 hours. The mixture was cooled to room temperature, diluted with water, extracted with EtOAc (10 mL×2), washed with brine, dried over Na₂SO₄, filtered, and concentrated to dryness. The resulting residue was purified by Prep-TLC (DCM:MeOH=15:1) to give the titled compound (8 mg, 9%). ¹H NMR (400 MHz, CD₃OD) δ 7.60-7.45 (m, 2H), 6.92 (s, 1H), 6.02-5.75 (m, 1H), 5.25-5.01 (m, 1H), 4.34 (s, 2H), 4.21 (s, 2H), 3.93 (s, 3H), 3.69 (s, 3H), 3.68-3.60 (m, 1H), 3.32-3.28 (m, 1H), 2.81-2.75 (m, 3H), 2.51-2.48 (m, 2H), 1.38-1.29 (m, 9H) ppm. MS: M/e 512 (M+1)⁺.

### Compound A78: 2-(6-((2S,5R)-4-(1-(4,4-difluorochroman-6-yl)ethyl)-2,5-dimethylpiperazin-1-yl)-9-ethyl-3-methyl-2-oxo-3,9-dihydro-2H-purin-8-yl)acetonitrile

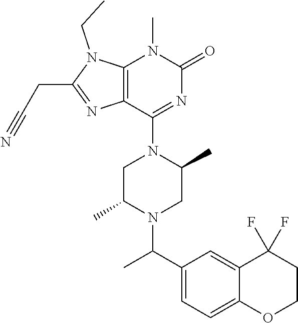 [SMILES: CCn1c(CC#N)nc2c(N3C[C@@H](C)N(C(C)c4ccc5c(c4)C(F)(F)CCO5)C[C@@H]3C)nc(=O)n(C)c21]

To a solution of 1-(4,4-difluorochroman-6-yl)ethan-1-ol (60 mg, 0.27 mmol), 2-(6-((2S,5R)-2,5-dimethylpiperazin-1-yl)-9-ethyl-3-methyl-2-oxo-3,9-dihydro-2H-purin-8-yl)acetonitrile (60 mg, 0.18 mmol) and (cyanomethyl) trimethyl phosphonium iodide (66 mg, 0.27 mmol) in CH₃CN (5 mL) was added DIPEA (116 mg, 0.9 mmol). The mixture was sealed in a bottle and heated at 100° C. for 16 hours. The mixture was cooled to room temperature, diluted with water, extracted with EtOAc (10 mL×2), washed with brine, dried over Na₂SO₄, filtered, and concentrated to dryness. The resulting residue was purified by Prep-TLC (DCM:MeOH=15:1) to give the titled compound (9 mg, 10%). ¹H NMR (400 MHz, CD₃OD) δ 7.61-7.58 (m, 1H), 7.44-7.38 (m, 1H), 6.92-6.86 (m, 1H), 6.01-5.70 (m, 1H), 5.25-4.99 (m, 1H), 4.38-4.33 (m, 4H), 4.23 (s, 2H), 3.71 (s, 3H), 3.68-3.63 (m, 1H), 3.50-3.30 (m, 1H), 3.23-3.13 (m, 1H), 3.00-2.50 (m, 2H), 2.48-2.45 (m, 2H), 1.46-1.28 (m, 9H), 0.99-0.67 (m, 3H) ppm. MS: M/e 526 (M+1)⁺.

### Compound A79: 2-(6-((2S,5R)-2,5-dimethyl-4-(1-(2,2,3,3-tetrafluoro-2,3-dihydrobenzo[b][1,4]dioxin-6-yl)ethyl)piperazin-1-yl)-3,9-dimethyl-2-oxo-3,9-dihydro-2H-purin-8-yl)acetonitrile

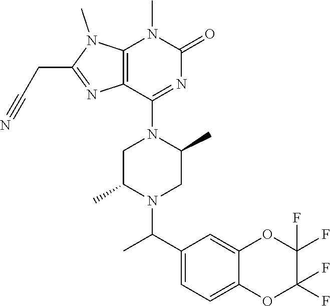 [SMILES: CC(c1ccc2c(c1)OC(F)(F)C(F)(F)O2)N1C[C@H](C)N(c2nc(=O)n(C)c3c2nc(CC#N)n3C)C[C@H]1C]

### Step A: 1-(2,2,3,3-tetrafluoro-2,3-dihydrobenzo[b][1,4]dioxin-6-yl)ethan-1-one

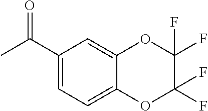 [SMILES: CC(=O)c1ccc2c(c1)OC(F)(F)C(F)(F)O2]

A solution of 6-bromo-2,2,3,3-tetrafluoro-2,3-dihydrobenzo[b][1,4]dioxine (450 mg, 1.57 mmol), tributyl(1-ethoxyvinyl)stannane (849 mg, 2.35 mmol) and Pd(PPh₃)₂Cl₂ (110 mg, 0.16 mmol) in toluene (5 ml) was stirred at 100° C. under N₂ overnight. After completed, the mixture was cooled to rt. HCl (4M in dioxane, 2 ml) was added and stirred at rt for 20 min. The reaction mixture was diluted with EtOAc (20 ml), washed with brine (10 ml), dried over Na₂SO₄ and then concentrated under reduced pressure. The residue was purified by flash column chromatography with 0-10% EtOAc in PE to give the titled compound (390 mg, 99%) as a colorless liquid. ¹H NMR (400 MHz, CDCl₃) δ 7.80 (dd, J=8.7 Hz, 1.7 Hz, 1H), 7.77 (d, J=1.7 Hz, 1H), 7.24 (d, J=8.7 Hz, 1H), 2.60 (s, 3H) ppm.

### Step B: 1-(2,2,3,3-tetrafluoro-2,3-dihydrobenzo[b][1,4]dioxin-6-yl)ethan-1-ol

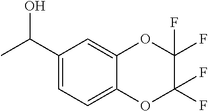 [SMILES: CC(O)c1ccc2c(c1)OC(F)(F)C(F)(F)O2]

To a solution of 1-(2,2,3,3-tetrafluoro-2,3-dihydrobenzo[b][1,4]dioxin-6-yl)ethan-1-one (390 mg, 1.56 mmol) in MeOH (4 ml) at rt, was added NaBH₄ (59 mg, 1.56 mmol). The solution was stirred at rt for 15 min. The reaction solution was diluted with EtOAc (15 ml), washed with brine (10 ml), dried over Na₂SO₄ and then evaporated to dryness to give the titled compound (390 mg, 100%) as a light-yellow oil. ¹H NMR (400 MHz, CDCl₃) δ 7.20 (d, J=1.7 Hz, 1H), 7.17 (dd, J=8.4 Hz, 1.7 Hz, 1H), 7.11 (d, J=8.4 Hz, 1H), 4.92 (q, J=6.2 Hz, 1H), 1.49 (d, J=6.4 Hz, 3H) ppm.

### Step C: 2-(6-((2S,5R)-2,5-dimethyl-4-(1-(2,2,3,3-tetrafluoro-2,3-dihydrobenzo[b][1,4]dioxin-6-yl)ethyl)piperazin-1-yl)-3,9-dimethyl-2-oxo-3,9-dihydro-2H-purin-8-yl)acetonitrile

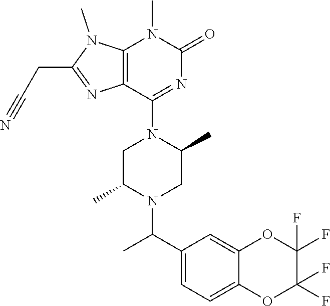 [SMILES: CC(c1ccc2c(c1)OC(F)(F)C(F)(F)O2)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (370 mg, 1.17 mmol), 1-(2,2,3,3-tetrafluoro-2,3-dihydrobenzo[b][1,4]dioxin-6-yl)ethan-1-ol (290 mg, 1.15 mmol), (cyanomethyl)trimethylphosphonium iodide (856 mg, 3.52 mmol) and DIPEA (1.52 g, 11.78 mmol) in CH₃CN (6 ml) was stirred at 100° C. overnight. The reaction was diluted with EtOAc (20 ml) and washed with brine (10 ml). The organic layer was concentrated under reduced pressure. The resulting residue was purified by flash column chromatography with 0-5% MeOH in DCM to give the titled Compound A79 (140 mg), which was further separated into Compound A79a (49 mg) and Compound A79b (61 mg) by chiral Prep-HPLC. The chiral separation conditions are shown below.

| | |
|---|---|
| Column | CHIRALPAK ID |
| Column Size | 2 cm × 25 cm, 5 um |
| Mobile Phase A | Hex(0.5% 2 mM NH3—MeOH) |
| Mobile Phase B | EtOH |
| Flow Rate | 20 mL/min |
| Wave Length | UV 220 nm |
| Temperature | 25° C. |
| Prep-HPLC Equipment | Prep-HPLC-Gilson |

Compound A79: ¹H NMR (400 MHz, DMSO-d6) δ 7.49-7.42 (m, 2H), 7.37 (t, J=8.4 Hz, 1H), 6.18-4.45 (m, 2H), 4.45-4.30 (m, 2H), 3.84 (s, 3H), 3.67 (q, J=6.3 Hz, 0.5H), 3.64 (s, 3H), 3.54-3.47 (m, 1H), 3.34-3.31 (m, 1.5H), 2.84-2.69 (m, 1.5H), 2.08 (d, J=12.0 Hz, 0.5H), 1.34 (s, 1.5H), 1.24 (dd, J=9.9, 6.5 Hz, 3H), 1.18 (d, J=6.5 Hz, 1.5H), 0.94 (d, J=6.1 Hz, 1.5H), 0.83 (d, J=6.5 Hz, 1.5H) ppm. MS: M/e 550 (M+1)⁺.

Compound A79a (the earlier peak): ¹H NMR (400 MHz, DMSO-d6) δ 7.49-7.42 (m, 2H), 7.38 (d, J=8.5 Hz, 1H), 6.10-4.40 (m, 2H), 4.41-4.30 (m, 2H), 3.83 (s, 3H), 3.67 (q, J=6.0 Hz, 1H), 3.64 (s, 3H), 3.31-3.28 (m, 1H), 2.85-2.70 (m, 3H), 1.34 (s, 3H), 1.23 (d, J=6.4 Hz, 3H), 0.83 (d, J=6.3 Hz, 3H) ppm. MS: M/e 550 (M+1)⁺.

Compound A79b (the later peak): ¹H NMR (400 MHz, DMSO-d6) δ 7.46-7.41 (m, 2H), 7.36 (d, J=8.3 Hz, 1H), 6.04-5.48 (m, 1H), 5.08-4.50 (m, 1H), 4.42-4.31 (m, 2H), 3.84 (s, 3H), 3.64 (s, 3H), 3.54-3.45 (m, 2H), 3.32-3.28 (m, 2H), 2.08 (d, J=11.8 Hz, 1H), 1.25 (d, J=6.3 Hz, 3H), 1.17 (d, J=5.8 Hz, 3H), 0.94 (d, J=6.1 Hz, 3H) ppm. MS: M/e 550 (M+1)⁺.

### Compound A80: 2-(6-((2S,5R)-4-(1-(3,3-difluoro-2,3-dihydrobenzo[b][1,4]dioxin-6-yl)ethyl)-2,5-dimethylpiperazin-1-yl)-9-ethyl-3-methyl-2-oxo-3,9-dihydro-2H-purin-8-yl)acetonitrile

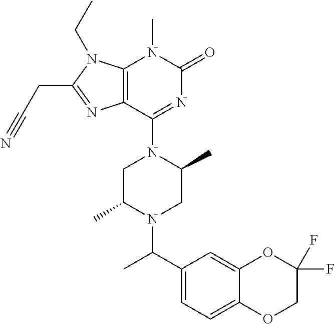 [SMILES: CCn1c(CC#N)nc2c(N3C[C@@H](C)N(C(C)c4ccc5c(c4)OC(F)(F)CO5)C[C@@H]3C)nc(=O)n(C)c21]

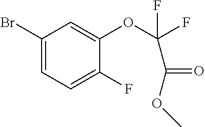 [SMILES: COC(=O)C(F)(F)Oc1cc(Br)ccc1F]

A solution of 5-bromo-2-fluorophenol (10 g, 0.052 mol), methyl 2-bromo-2,2-difluoroacetate (14.8 g, 0.078 mol), DBU (15.9 g, 0.10 mol) in DMF (60 ml) was stirred at 70° C. overnight. The solution was poured into water (50 ml) and then extracted with EtOAc (50 ml×2). The organic layer was washed with brine and concentrated. The residue was purified by flash column chromatography with 0-1% EtOAc in PE to give the titled compound (8.4 g, 53%). ¹H NMR (400 MHz, CDCl₃) δ 7.48 (d, J=6.3 Hz, 1H), 7.42-7.35 (m, 1H), 7.08 (t, J=9.2 Hz, 1H), 3.98 (s, 3H) ppm

### Step B: 2-(5-bromo-2-fluorophenoxy)-2,2-difluoroethan-1-ol

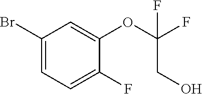 [SMILES: OCC(F)(F)Oc1cc(Br)ccc1F]

To a solution of methyl 2-(5-bromo-2-fluorophenoxy)-2,2-difluoroacetate (8.4 g, 0.028 mol) in THE (80 ml), was added LiAlH₄ (1M in THF, 56 ml, 56 mmol) dropwise. The solution was stirred at RT overnight. The solution was quenched with water (50 ml) and separated. The organic layer was concentrated. The residue was purified by flash column chromatography with 0-10% EtOAc in PE to give the title compound (5 g, 65%). ¹H NMR (400 MHz, CD₃OD) δ 7.34 (t, J=7.7 Hz, 1H), 7.28-7.16 (m, 2H), 3.94 (t, J=9.8 Hz, 2H) ppm

### Step C: 3-(1,1-difluoro-2-hydroxyethoxy)-4-fluorobenzonitrile

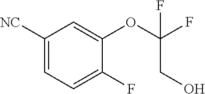 [SMILES: N#Cc1ccc(F)c(OC(F)(F)CO)c1]

A solution of 2-(5-bromo-2-fluorophenoxy)-2,2-difluoroethan-1-ol (2.8 g, 0.010 mol), Pd(PPh₃)₄ (1.2 g, 0.0010 mol) and Zn(CN)₂ (2.4 g, 0.021 mol) in DMF (20 ml) was stirred at 120° C. overnight under N₂. The solution was poured into water (40 ml) and extracted with EtOAc (30 ml×2). The organic layer was washed with brine (20 ml), dried and concentrated. The residue was purified by flash column chromatography with 0-20% EtOAc in PE to give the titled compound (390 mg, 17%). MS: M/e 218 (M+1)⁺.

### Step D: 3,3-difluoro-2,3-dihydrobenzo[b][1,4]dioxine-6-carbonitrile

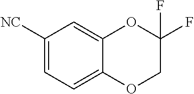 [SMILES: N#Cc1ccc2c(c1)OC(F)(F)CO2]

A solution of 3-(1,1-difluoro-2-hydroxyethoxy)-4-fluorobenzonitrile (260 mg, 1.20 mmol) and NaH (60%, 72 mg, 1.8 mmol) in THE (10 ml) was stirred at RT for 30 min. The mixture was diluted with EtOAc (10 ml), washed with brine (10 ml), dried and concentrated. The residue was purified by flash column chromatography with 0-15% EtOAc in PE to give the titled compound (150 mg, 63%). ¹H NMR (400 MHz, CDCl₃) δ 7.36 (d, J=6.9 Hz, 2H), 7.11 (d, J=8.9 Hz, 1H), 4.33 (t, J=5.9 Hz, 2H) ppm

### Step E: 1-(3,3-difluoro-2,3-dihydrobenzo[b][1,4]dioxin-6-yl)ethan-1-one

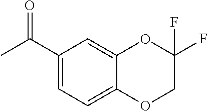

A solution of 3,3-difluoro-2,3-dihydrobenzo[b][1,4]dioxine-6-carbonitrile (200 mg, 1.1 mmol), MeMgBr (1M in THF, 1.52 ml, 1.52 mmol) and CuBr (10.9 mg, 0.076 mmol) in THF (6 ml) was stirred at 60° C. overnight. The mixture was cooled to RT, HCl (3N, 1 ml) was added and stirred for 30 min. The mixture was diluted with EtOAc (15 ml), washed with brine (10 ml), dried and concentrated. The residue was purified by flash column chromatography with 0-15% EtOAc in PE to give the titled compound (170 mg, 82%). ¹H NMR (400 MHz, CDCl₃) δ 7.67 (d, J=8.4 Hz, 2H), 7.08 (d, J=8.4 Hz, 1H), 4.32 (t, J=5.9 Hz, 2H), 2.57 (s, 3H) ppm

### Step F: 1-(3,3-difluoro-2,3-dihydrobenzo[b][1,4]dioxin-6-yl)ethan-1-ol

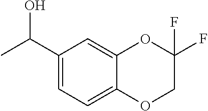

A solution of 1-(3,3-difluoro-2,3-dihydrobenzo[b][1,4]dioxin-6-yl)ethan-1-one (170 mg, 0.79 mmol) and NaBH₄ (30 mg, 0.79 mmol) in MeOH (4 ml) was stirred at RT for 40 min. The solution was diluted with EtOAc (10 ml), washed with brine (10 ml), dried and evaporated to dryness to give the titled compound (170 mg, 100%), which was used directly for the next step without further purification. MS: M/e 217 (M+1)⁺.

### Step G: 2-(6-((2S,5R)-4-(1-(3,3-difluoro-2,3-dihydrobenzo[b][1,4]dioxin-6-yl)ethyl)-2,5-dimethylpiperazin-1-yl)-9-ethyl-3-methyl-2-oxo-3,9-dihydro-2H-purin-8-yl)acetonitrile

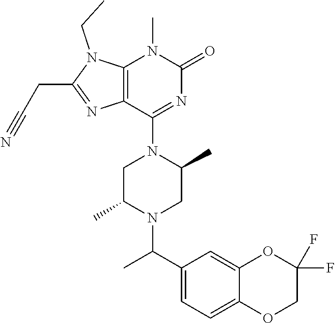 [SMILES: CCn1c(CC#N)nc2c(N3C[C@@H](C)N(C(C)c4ccc5c(c4)OC(F)(F)CO5)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (261 mg, 0.79 mmol), 1-(3,3-difluoro-2,3-dihydrobenzo[b][1,4]dioxin-6-yl)ethan-1-ol (170 mg, 0.79 mmol), (cyanomethyl)trimethylphosphonium iodide (579 mg, 2.38 mmol) and DIPEA (1.02 g, 7.91 mmol) in MeCN (4 ml) was stirred at 100° C. overnight. The reaction was diluted with EtOAc (10 ml) and washed with brine (10 ml). The organic layer was concentrated under reduced pressure. The resulting residue was purified by flash column chromatography with 0-5% MeOH in DCM to give the titled Compound A80 (100 mg), which was further separated into Compound A80a (29 mg) and Compound A80b (32 mg) by chiral prep-HPLC. The chiral separation conditions are shown below.

| | |
|---|---|
| Column | CHIRALPAK IH |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

### Prep-HPLC Equipment Prep-HPLC-Gilson

Compound A80: ¹H NMR (400 MHz, CD₃OD) δ 7.17-7.07 (m, 2H), 7.06-6.97 (m, 1H), 6.30-4.95 (m, 2H), 4.40-4.32 (m, 4H), 4.23 (s, 2H), 3.80-3.74 (m, 3.5H), 3.74-3.48 (m, 2H), 3.07-2.66 (m, 2H), 2.29 (d, J=12 Hz, 0.5H), 1.46 (t, J=7.1 Hz, 4.5H), 1.39-1.32 (m, 3H), 1.27 (d, J=5.3 Hz, 1.5H), 1.07 (d, J=6.0 Hz, 1.5H), 0.96 (d, J=4.8 Hz, 1.5H) ppm. MS: M/e 528 (M+1)⁺.

Compound A80a (the earlier peak): ¹H NMR (400 MHz, DMSO-d6) δ [0888]7.16 (s, 1H), 7.10 (q, J=5.5 Hz, 2H), 6.10-5.40 (m, 1H), 5.25-4.92 & 4.51-4.44 (m, 1H), 4.62-4.53 (m, 2H), 4.44-4.33 (m, 2H), 4.32-4.16 (m, 2H), 3.61 (s, 3H), 3.56 (q, J=5.3 Hz, 1H), 3.33-3.28 (m, 2H), 2.80-2.72 (m, 2H), 1.45-1.27 (m, 6H), 1.21 (d, J=6.1 Hz, 3H), 0.82 (d, J=5.4 Hz, 3H) ppm. MS: M/e 528 (M+1)⁺.

Compound A80b (the later peak): ¹H NMR (400 MHz, DMSO-d6) δ 7.18-7.04 (m, 3H), 5.93-5.63 (m, 1H), 5.00-4.65 (m, 1H), 4.56 (t, J=5.7 Hz, 2H), 4.47-4.35 (m, 2H), 4.33-4.20 (m, 2H), 3.61 (s, 3H), 3.53-3.40 (m, 2H), 3.33-3.29 (m, 2H), 2.14 (d, J=9.7 Hz, 1H), 1.34 (t, J=5.7 Hz, 3H), 1.23 (d, J=5.7 Hz, 3H), 1.21-1.12 (m, 3H), 1.00-0.88 (m, 3H) ppm. MS: M/e 528 (M+1)⁺.

### Compound A81: 2-(6-((2S,5R)-4-(1-(2,2-dimethyl-4-oxochroman-7-yl)ethyl)-2,5-dimethyl piperazin-1-yl)-9-ethyl-3-methyl-2-oxo-3,9-dihydro-2H-purin-8-yl)acetonitrile

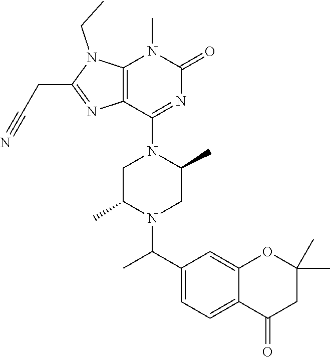 [SMILES: CCn1c(CC#N)nc2c(N3C[C@@H](C)N(C(C)c4ccc5c(c4)OC(C)(C)CC5=O)C[C@@H]3C)nc(=O)n(C)c21]

### Step A: 7-acetyl-2,2-dimethylchroman-4-one

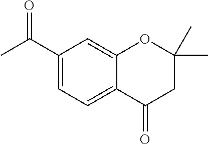 [SMILES: CC(=O)c1ccc2c(c1)OC(C)(C)CC2=O]

A mixture of 7-bromo-2,2-dimethylchroman-4-one (510 mg, 2.0 mmol), tributyl(1-ethoxyvinyl)stannane (1.45 g, 4.0 mmol) and Pd(PPh₃)₂Cl₂ (70 mg, 0.1 mmol) in toluene (5 mL) was stirred at 100° C. under N₂ for 16 hrs. The mixture was cooled and diluted with EtOAc (10 mL), washed with brine (10 mL×2). The organic layer was added HCl/Dioxane (4M, 5 mL), stirred for 5 min and washed with brine (10 mL×2), NaHCO₃ (10 mL×2), brine (10 mL), dried over Na₂SO₄, filtered and concentrated to dryness. The resulted residue was purified by flash column chromatography to give the titled compound (458 mg, 95%). ¹H NMR (400 MHz, CDCl₃) δ 7.94 (d, J=8.0 Hz, 1H), 7.56-7.46 (m, 2H), 2.77 (s, 2H), 2.61 (s, 3H), 1.48 (s, 6H) ppm.

### Step B: 7-(1-hydroxyethyl)-2,2-dimethylchroman-4-one

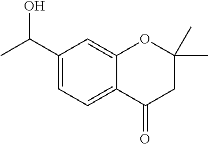 [SMILES: CC(O)c1ccc2c(c1)OC(C)(C)CC2=O]

To a solution of 7-acetyl-2,2-dimethylchroman-4-one (458 mg, 2.0 mmol) in MeOH (5 mL) was added NaBH₄ (38 mg, 1.0 mmol) at room temperature and the mixture was stirred at rt for 2 hours. The mixture was treated with NaHCO₃ (15 mL), extracted with CH₂Cl₂ (10 mL×5). The combined extracts was washed with brine (10 mL), dried over Na₂SO₄ and concentrated to dryness and purified by flash column chromatography to give the title compound (307 mg, 67%). ¹H NMR (400 MHz, DMSO-d6) δ 7.66 (d, J=8.0 Hz, 1H), 6.99 (d, J=8.0 Hz, 1H), 6.92 (s, 1H), 5.31 (d, J=4.4 Hz, 1H), 4.75-4.63 (m, 1H), 2.75 (s, 2H), 1.38 (s, 6H), 1.30 (d, J=6.4 Hz, 3H) ppm.

### Step C: 2-(6-((2S,5R)-4-(1-(2,2-dimethyl-4-oxochroman-7-yl)ethyl)-2,5-dimethylpiperazin-1-yl)-9-ethyl-3-methyl-2-oxo-3,9-dihydro-2H-purin-8-yl)acetonitrile

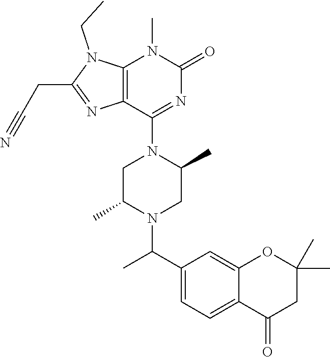 [SMILES: CCn1c(CC#N)nc2c(N3C[C@@H](C)N(C(C)c4ccc5c(c4)OC(C)(C)CC5=O)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol), 7-(1-hydroxyethyl)-2,2-dimethylchroman-4-one (134 mg, 0.61 mmol) and (cyanomethyl)trimethylphosphonium iodide (150 mg, 0.61 mmol) in CH₃CN (2 mL) was added DIPEA (155 mg, 1.2 mmol). The mixture was stirred at 100° C. in a sealed tube for 16 hours. The mixture was cooled and diluted with EtOAc (20 mL), washed with brine (10 mL×3), dried over Na₂SO₄, filtered and concentrated under reduced pressure. The resulting residue was purified by flash column chromatography and further purified by Prep-HPLC (Method A) to give the titled compound (68 mg, 43%). ¹H NMR (400 MHz, CD₃OD) δ 7.82-7.71 (m, 1H), 7.12-7.03 (m, 1H), 7.00 (s, 1H), 6.38-5.45 (m, 1H), 5.41-4.50 (m, 1H), 4.37 (q, J=7.2 Hz, 2H), 4.23 (s, 2H), 3.76 (s, 3H), 3.68-3.37 (m, 2H), 2.96-2.15 (m, 5H), 1.48-1.27 (m, 15H), 1.08-0.88 (m, 3H) ppm. MS: M/e 532 (M+1)⁺.

### Compound A83: 2-(6-((2S,5R)-2,5-dimethyl-4-(1-(2-methyl-1,3-dioxoisoindolin-5-yl)ethyl) piperazin-1-yl)-9-ethyl-3-methyl-2-oxo-3,9-dihydro-2H-purin-8-yl)acetonitrile

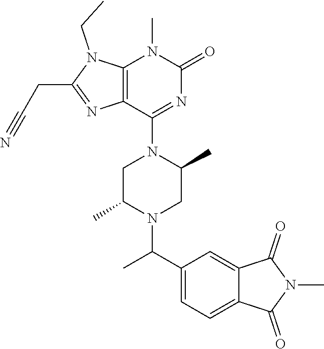 [SMILES: CCn1c(CC#N)nc2c(N3C[C@@H](C)N(C(C)c4ccc5c(c4)C(=O)N(C)C5=O)C[C@@H]3C)nc(=O)n(C)c21]

### Step A: 5-bromo-2-methylisoindoline-1,3-dione

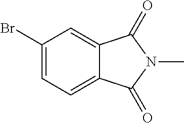

To a 0° C. solution of 5-bromoisoindoline-1,3-dione (2.26 g, 10 mmol) in DMF (40 mL) was added NaH (600 mg, 15 mmol) in portions. The resulted mixture was stirred at 0° C. for 10 min. Iodomethane (2.13 g, 15 mmol) was added and the mixture was stirred at room temperature for 2 hours. The mixture was quenched with aqueous solution of NH₄Cl (100 mL). EtOAc (100 mL) was added and the mixture was washed with brine (100 mL×3), dried over Na₂SO₄, filtered and concentrated to dryness. The resulted residue was purified by column chromatography to give the titled compound (1.51 g, 63%). ¹H NMR (400 MHz, DMSO-d6) δ 8.06 (s, 1H), 8.02 (d, J=8.0 Hz, 1H), 7.79 (d, J=8.0 Hz, 1H), 3.03 (s, 3H) ppm.

### Step B: 5-acetyl-2-methylisoindoline-1,3-dione

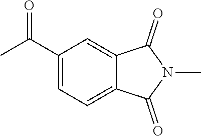

A mixture of 5-bromo-2-methylisoindoline-1,3-dione (1.0 g, 4.16 mmol), tributyl(1-ethoxyvinyl)stannane (3.0 g, 8.33 mmol) and Pd(PPh₃)₂Cl₂ (140 mg, 0.2 mmol) in toluene (10 mL) was stirred at 100° C. under N₂ for 16 hrs. The mixture was cooled and diluted with EtOAc (50 mL), washed with brine (20 mL×2). The mixture was filtered through a celite pad and the organic layer was treated with HCl/Dioxane (4M, 5 mL), and washed with brine (20 mL×2), NaHCO₃ (20 mL×2), dried over Na₂SO₄, filtered and concentrated to dryness. The resulted residue was purified by flash column chromatography to give the titled compound (800 mg, 93%). ¹H NMR (400 MHz, CDCl₃) δ 8.38 (s, 1H), 8.32 (d, J=7.6 Hz, 1H), 7.95 (d, J=8.0 Hz, 1H), 3.22 (s, 3H), 2.70 (s, 3H) ppm.

### Step C: 5-(1-hydroxyethyl)-2-methylisoindoline-1,3-dione

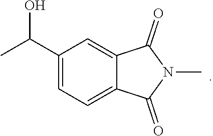

To a solution of 5-acetyl-2-methylisoindoline-1,3-dione (800 mg, 3.9 mmol) in MeOH (10 mL) was added NaBH₄ (80 mg, 2.1 mmol) at room temperature and the mixture was stirred at rt for 2 hours. The mixture was treated with NaHCO₃ (50 mL), extracted with EtOAc (50 mL×2). The combined extracts was washed with brine (50 mL×2), dried over Na₂SO₄ and concentrated to dryness and purified by flash column chromatography to give the title compound (300 mg, 37%). ¹H NMR (400 MHz, DMSO-d6) δ 7.88-7.80 (m, 3H), 5.50 (s, 1H), 4.89 (q, J=6.0 Hz, 1H), 3.03 (s, 3H), 1.36 (d, J=6.4 Hz, 3H) ppm.

### Step D: 2-(6-((2S,5R)-2,5-dimethyl-4-(1-(2-methyl-1,3-dioxoisoindolin-5-yl)ethyl)piperazin-1-yl)-9-ethyl-3-methyl-2-oxo-3,9-dihydro-2H-purin-8-yl)acetonitrile

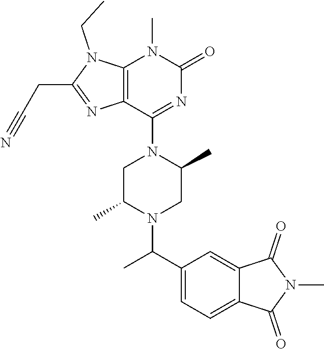 [SMILES: CCn1c(CC#N)nc2c(N3C[C@@H](C)N(C(C)c4ccc5c(c4)C(=O)N(C)C5=O)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (82 mg, 0.25 mmol), 5-(1-hydroxyethyl)-2-methylisoindoline-1,3-dione (100 mg, 0.49 mmol) and (cyanomethyl)trimethylphosphonium iodide (121 mg, 0.50 mmol) in CH₃CN (2 mL) was added DIPEA (129 mg, 1.0 mmol). The mixture was stirred at 100° C. in a sealed tube for 16 hours. The mixture was cooled and diluted with EtOAc (20 mL), washed with brine (10 mL×3), dried over Na₂SO₄, filtered and concentrated under reduced pressure. The resulting residue was purified by flash column chromatography and further purified by Prep-HPLC(Method A) to give the titled compound (18 mg, 14%). ¹H NMR (400 MHz, CD₃OD) δ 7.92 (s, 1H), 7.86-7.74 (m, 2H), 6.16-4.50 (m, 2H), 4.44-4.30 (m, 2H), 4.29-4.08 (m, 2H), 3.89-3.57 (m, 5H), 3.12 (s, 3H), 3.00-2.57 (m, 2.5H), 2.23-2.08 (m, 0.5H), 1.55-1.20 (m, 9H), 1.11-0.88 (m, 3H) ppm. MS: M/e 517 (M+1)⁺.

### Compound A85: 2-(6-((2S,5R)-4-(1-(2,2-dimethylbenzo[d][1,3]dioxol-5-yl)ethyl)-2,5-dimethyl piperazin-1-yl)-9-ethyl-3-methyl-2-oxo-3,9-dihydro-2H-purin-8-yl)acetonitrile

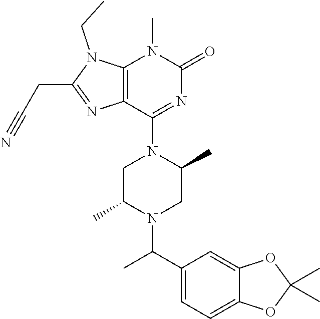 [SMILES: CCn1c(CC#N)nc2c(N3C[C@@H](C)N(C(C)c4ccc5c(c4)OC(C)(C)O5)C[C@@H]3C)nc(=O)n(C)c21]

To a solution of 1-(2,2-dimethylbenzo[d][1,3]dioxol-5-yl)ethan-1-ol (116 mg, 0.6 mmol), 2-(6-((2S,5R)-2,5-dimethylpiperazin-1-yl)-9-ethyl-3-methyl-2-oxo-3,9-dihydro-2H-purin-8-yl)acetonitrile (99 mg, 0.3 mmol) and (cyanomethyl)trimethylphosphonium iodide (145 mg, 0.6 mmol) in CH₃CN (3 mL) was added DIPEA (155 mg, 1.2 mmol). The mixture was sealed in a bottle and heated at 100° C. overnight. Then the mixture was cooled to room temperature, diluted with water, extracted with EtOAc (50 mL×2), washed with brine, dried over Na₂SO₄, filtered, and concentrated to dryness. The resulting residue was purified by Prep-TLC (DCM:MeOH=13:1) to give the titled Compound A85 (25 mg), which was further separated into Compound A85a (11 mg) and Compound A85b (10 mg) by chiral Prep-HPLC. The chiral separation conditions are shown below.

| | |
|---|---|
| Column | CHIRALPAK IF |
| Column Size | 2 cm × 25 cm, 5 um |
| Mobile Phase A | MtBE |
| Mobile Phase B | ACN |
| Flow Rate | 20 mL/min |
| Wave Length | UV 220 nm |
| Temperature | 25° C. |
| Prep-HPLC Equipment | Prep-HPLC-Gilson |

Compound A85: ¹HNMR (400 MHz, CDCl₃) δ 6.82 (d, J=9.4 Hz, 1H), 6.75-6.69 (m, 1H), 6.63 (d, J=7.9 Hz, 1H), 6.00-4.50 (m, 2H), 4.30 (d, J=6.7 Hz, 2H), 3.87 (d, J=23.4 Hz, 2H), 3.77 (s, 3H), 3.58-3.18 (m, 3H), 2.95-2.74 (m, 1H), 2.58-2.27 (m, 1H), 1.69 (d, J=8.6 Hz, 6H), 1.49 (dd, J=26.0, 18.9 Hz, 5H), 1.26 (dd, J=15.2, 6.4 Hz, 4H), 1.05-0.84 (m, 3H) ppm. MS: M/e 506 (M+1)⁺.

Compound A85a (the earlier peak) ¹H NMR (400 MHz, DMSO-d6) δ 6.82 (s, 1H), 6.74 (q, J=7.9 Hz, 2H), 5.94-5.60 (m, 1H), 4.96-4.58 (m, 1H), 4.40 (s, 2H), 4.26 (d, J=7.2 Hz, 2H), 3.61 (s, 4H), 3.46 (s, 1H), 3.34 (s, 1H), 2.49-2.39 (m, 1H), 2.18 (d, J=12.3 Hz, 1H), 1.62 (s, 6H), 1.34 (t, J=7.0 Hz, 3H), 1.22 (d, J=6.5 Hz, 3H), 1.16 (s, 3H), 0.93 (s, 3H) ppm. MS: M/e 506 (M+1)⁺.

Compound A85b (the later peak) ¹H NMR (400 MHz, DMSO-d6) δ 6.84 (s, 1H), 6.75 (q, J=8.0 Hz, 2H), 6.1-5.40 (m, 1H), 5.15-4.45 (m, 1H), 4.38 (s, 2H), 4.25 (s, 2H), 3.61 (s, 3H), 3.47 (d, J=6.3 Hz, 1H), 3.30-2.95 (m, 1H), 2.90-2.62 (m, 3H), 1.63 (d, J=5.0 Hz, 7H), 1.34 (s, 6H), 1.19 (d, J=6.2 Hz, 3H), 0.81 (d, J=5.9 Hz, 3H) ppm. MS: M/e 506 (M+1)⁺.

### Compound A86: 2-(9-cyclopropyl-6-((2S,5R)-4-(1-(3,3-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile

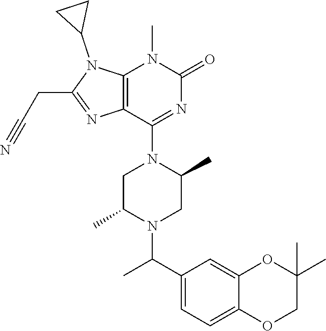 [SMILES: CC(c1ccc2c(c1)OC(C)(C)CO2)N1C[C@H](C)N(c2nc(=O)n(C)c3c2nc(CC#N)n3C2CC2)C[C@H]1C]

### Step A: 6-(cyclopropylamino)-1-methylpyrimidine-2,4 (1H,3H)-dione

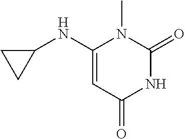

To a stirred mixture of 6-chloro-1-methylpyrimidine-2,4 (1H,3H)-dione (4.8 g, 30 mmol) in EtOH (50 mL) was added cyclopropanamine (3.42 g, 60 mmol). After the addition, the reaction mixture was stirred at 60° C. overnight. The reaction mixture was cooled to room temperature and filtered. The caked was collected and dried to give the titled compound (4.2 g, 77%). MS: M/e 182 (M+1)⁺.

### Step B: 6-(cyclopropylamino)-1-methyl-5-nitrosopyrimidine-2,4 (1H,3H)-dione

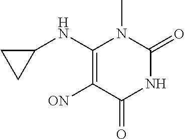

To a stirred mixture of 6-(cyclopropylamino)-1-methylpyrimidine-2,4 (1H,3H)-dione (4.2 g, 23.2 mmol) in H₂O (50 mL) was added NaNO₂ (1.76 g, 25.5 mmol) at 0˜5° C. Then AcOH (4.2 g, 69.6 mmol) was added dropwise at that temperature. After then, the mixture was stirred for 30 minutes. The reaction mixture was filtered and the cake was collected, dried to give the titled compound (3.3 g, 68%). MS: M/e 211 (M+1)⁺.

### Step C: 5-amino-6-(cyclopropylamino)-1-methylpyrimidine-2,4 (1H,3H)-dione

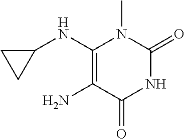

To a stirred mixture of 6-(cyclopropylamino)-1-methyl-5-nitrosopyrimidine-2,4 (1H,3H)-dione (3.3 g, 15.7 mmol) in H₂O (50 mL) was added Na₂S₂O₄ (8.2 g, 47.1 mmol) portionwise at 20-30° C. After the addition, the reaction mixture was stirred for 30 minutes. The reaction mixture was cooled in a icebox for several hours and filtered. The cake was collected, dried to give the titled compound (2 g, 65%). MS: M/e 197 (M+1)⁺.

### Step D: 8-((benzyloxy)methyl)-9-cyclopropyl-3-methyl-3,9-dihydro-1H-purine-2,6-dione

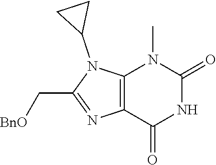 [SMILES: Cn1c(=O)[nH]c(=O)c2nc(COCc3ccccc3)n(C3CC3)c21]

A mixture of 5-amino-6-(cyclopropylamino)-1-methylpyrimidine-2,4 (1H,3H)-dione (2 g, 10.2 mmol) in 2-(benzyloxy)acetic acid (1.86 g, 11.2 mmol) was stirred at 240° C. for 1.5 hour. The mixture was allowed cool to room temperature and dissolved in aq.NaOH (2.0 M, 20 mL), then acidified to pH=5˜6 with aq.HCl and filtered. The cake was collected, dried to give the titled compound (2 g, 61%). MS: M/e 327 (M+1)⁺.

### Step E: tert-butyl (2R,5S)-4-(8-((benzyloxy)methyl)-9-cyclopropyl-3-methyl-2-oxo-3,9-dihydro-2H-purin-6-yl)-2,5-dimethylpiperazine-1-carboxylate

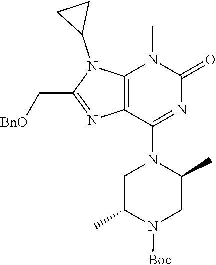 [SMILES: C[C@@H]1CN(c2nc(=O)n(C)c3c2nc(COCc2ccccc2)n3C2CC2)[C@@H](C)CN1C(=O)OC(C)(C)C]

To a stirred mixture of 8-((benzyloxy)methyl)-9-cyclopropyl-3-methyl-3,9-dihydro-1H-purine-2,6-dione (978 mg, 3 mmol) and tert-butyl (2R,5S)-2,5-dimethylpiperazine-1-carboxylate (1.28 g, 6 mmol) in CH₃CN (15 mL) was added BOP (2.65 g, 6 mmol), followed by DBU (918 mg, 6 mmol). After the addition, the reaction mixture was concentrated to give the residue, which was dissolved in EtOAc (50 mL), washed with H₂O (20 mL), brine, dried over Na₂SO₄, concentrated to dryness. The resulting residue was purified by flash column chromatography to give the titled compound (0.84 g, 54%). MS: M/e 523 (M+1)⁺.

### Step F: tert-butyl (2R,5S)-4-(9-cyclopropyl-8-(hydroxymethyl)-3-methyl-2-oxo-3,9-dihydro-2H-purin-6-yl)-2,5-dimethylpiperazine-1-carboxylate

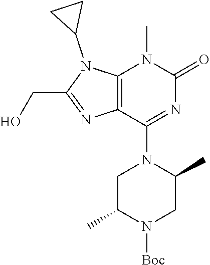 [SMILES: C[C@@H]1CN(c2nc(=O)n(C)c3c2nc(CO)n3C2CC2)[C@@H](C)CN1C(=O)OC(C)(C)C]

To a stirred solution of tert-butyl (2R,5S)-4-(8-((benzyloxy)methyl)-9-cyclopropyl-3-methyl-2-oxo-3,9-dihydro-2H-purin-6-yl)-2,5-dimethylpiperazine-1-carboxylate (0.84 g, 1.6 mmol) in MeOH/AcOH (80 mL/2 mL) was added Pd/C (200 mg). After the addition, the reaction mixture was stirred over a weekend under H₂ (1 atm). The reaction mixture was filtered, and filtrate was concentrated to give the titled compound (crude, 100%). MS: M/e 433 (M+1)⁺.

### Step G: tert-butyl (2R,5S)-4-(8-(cyanomethyl)-9-cyclopropyl-3-methyl-2-oxo-3,9-dihydro-2H-purin-6-yl)-2,5-dimethylpiperazine-1-carboxylate

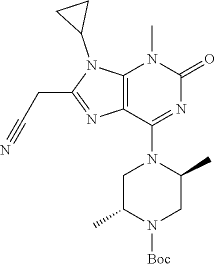 [SMILES: C[C@@H]1CN(c2nc(=O)n(C)c3c2nc(CC#N)n3C2CC2)[C@@H](C)CN1C(=O)OC(C)(C)C]

To a stirred solution of tert-butyl (2R,5S)-4-(9-cyclopropyl-8-(hydroxymethyl)-3-methyl-2-oxo-3,9-dihydro-2H-purin-6-yl)-2,5-dimethylpiperazine-1-carboxylate (crude, 1.6 mmol) in CH₂Cl₂ (10 mL) was added SOCl₂ (0.21 g, 1.77 mmol). After stirred for 10 min, the reaction mixture was quenched with aq.NaHCO₃, washed with brine, dried over Na₂SO₄, concentrated to give the intermediate, dissolved in CH₂Cl₂ (10 mL), TBAF (1.0 M, 3.2 mL, 3.2 mmol) was added followed by TMSCN (0.32 g, 3.2 mmol). After the addition, the reaction mixture was stirred for 3 hours. The reaction mixture was concentrated to give the residue, treated with H₂O and extracted with EtOAc (15 mL×3). The combined organic layers were washed with brine, dried over Na₂SO₄, concentrated to dryness. The resulting residue was purified by flash column chromatography to give the titled compound (330 mg, 47%). MS: M/e 442 (M+1)⁺.

### Step H: 2-(9-cyclopropyl-6-((2S,5R)-2,5-dimethylpiperazin-1-yl)-3-methyl-2-oxo-3,9-dihydro-2H-purin-8-yl)acetonitrile

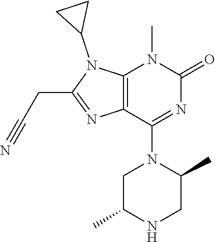 [SMILES: C[C@@H]1CN(c2nc(=O)n(C)c3c2nc(CC#N)n3C2CC2)[C@@H](C)CN1]

To a stirred solution of tert-butyl (2R,5S)-4-(8-(cyanomethyl)-9-cyclopropyl-3-methyl-2-oxo-3,9-dihydro-2H-purin-6-yl)-2,5-dimethylpiperazine-1-carboxylate (330 mg, 0.748 mmol) in EtOAc (10 mL) was added dioxane/HCl (g) (4.0 M, 4 mL). After the addition, the reaction mixture was stirred for 4 hours. The reaction mixture was concentrated to give the residue, which was treated with CH₃CN/aq.NaHCO₃ (20 mL/3 mL), stirred for 10 min and filtered. The filtrate was concentrated, dried to give the titled compound (240 mg, 97%), which was directly used to the next step. MS: M/e 342 (M+1)⁺.

### Step I: 2-(9-cyclopropyl-6-((2S,5R)-4-(1-(3,3-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile

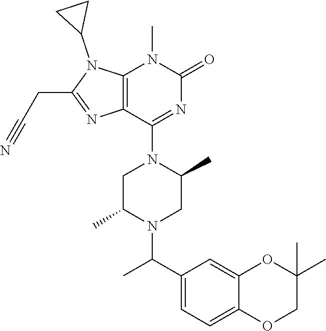 [SMILES: CC(c1ccc2c(c1)OC(C)(C)CO2)N1C[C@H](C)N(c2nc(=O)n(C)c3c2nc(CC#N)n3C2CC2)C[C@H]1C]

A mixture of 2-(9-cyclopropyl-6-((2S,5R)-2,5-dimethylpiperazin-1-yl)-3-methyl-2-oxo-3,9-dihydro-2H-purin-8-yl)acetonitrile (240 mg, 0.7 mmol), 1-(3,3-dimethyl-2,3-dihydrobenzo[b][1,4]dioxin-6-yl)ethan-1-ol (439 mg, 2.1 mmol), (cyanomethyl)trimethylphosphonium iodide (680 mg, 2.8 mmol) and DIPEA (903 mg, 7 mmol) in CH₃CN (6 mL) was stirred overnight at 100° C. in a sealed tube. The reaction mixture was diluted with EtOAc (20 mL), washed with H₂O, brine, dried over Na₂SO₄, concentrated to dryness. The resulting residue was purified by Pre-TLC (CH₂Cl₂/MeOH=10:1) to give the desired compound, and further purified by Prep-HPLC (Method A) to give the titled compound (60 mg, 18%). ¹H NMR (400 MHz, CD₃OD) δ 6.86-6.73 (m, 3H), 6.25-4.97 (m, 2H), 4.31-4.19 (m, 1H), 3.88 (d, J=3.2 Hz, 2H), 3.83 (s, 3H), 3.64-3.39 (m, 3.5H), 3.04-2.82 (m, 1.5H), 2.65 (s, 1H), 2.31 (d, J=12.4 Hz, 0.5H), 2.04-1.94 (m, 0.5H), 1.48-1.34 (m, 3H), 1.34-1.28 (m, 9H), 1.27-1.13 (m, 4H), 1.05-0.88 (m, 3H) ppm. MS: M/e 532 (M+1)⁺.

### Compound A87: 2-(9-(2,2-difluoroethyl)-6-((2S,5R)-4-(1-(3,3-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile

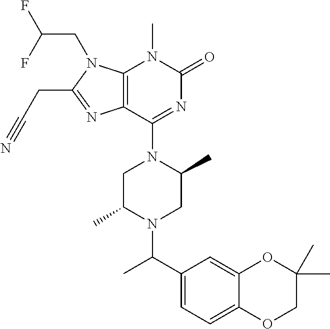 [SMILES: CC(c1ccc2c(c1)OC(C)(C)CO2)N1C[C@H](C)N(c2nc(=O)n(C)c3c2nc(CC#N)n3CC(F)F)C[C@H]1C]

### Step A: 6-((2,2-difluoroethyl)amino)-1-methylpyrimidine-2,4 (1H,3H)-dione

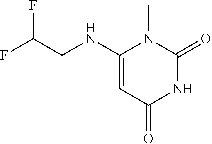

To a stirred mixture of 6-chloro-1-methylpyrimidine-2,4 (1H,3H)-dione (4.8 g, 30 mmol) in EtOH (50 mL) was added 2,2-difluoroethan-1-amine (4.8 g, 60 mmol). After the addition, the reaction mixture was stirred at 60° C. overnight. The reaction mixture was cooled to room temperature and filtered. The caked was collected and dried to give the titled compound (6.2 g, 100%). MS: M/e 206 (M+1)⁺.

### Step B: 6-((2,2-difluoroethyl)amino)-1-methyl-5-nitrosopyrimidine-2,4 (1H,3H)-dione

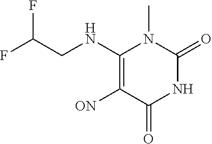

To a stirred mixture of 6-((2,2-difluoroethyl)amino)-1-methylpyrimidine-2,4 (1H,3H)-dione (6.1 g, 29.7 mmol) in H₂O (80 mL) was added NaNO₂ (2.3 g, 32.7 mmol) at 0˜5° C. Then AcOH (5.3 g, 89.1 mmol) was added dropwise at that temperature. After then, the mixture was stirred for 30 minutes. The reaction mixture was filtered, and the cake was collected, dried to give the titled compound (7.6 g, 100%). MS: M/e 235 (M+1)⁺.

### Step C: 5-amino-6-((2,2-difluoroethyl)amino)-1-methylpyrimidine-2,4 (1H,3H)-dione

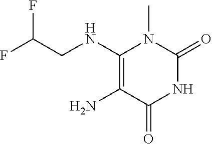

To a stirred mixture of 6-((2,2-difluoroethyl)amino)-1-methyl-5-nitrosopyrimidine-2,4 (1H,3H)-dione (7.6 g, 32.5 mmol) in H₂O (80 mL) was added Na₂S₂O₄ (17 g, 97.4 mmol) portionwise at 20˜30° C. After the addition, the reaction mixture was stirred for 30 minutes. The reaction mixture was cooled in a icebox for several hours and filtered. The cake was collected, dried to give the titled compound (2.9 g, 40.5%). MS: M/e 221 (M+1)⁺.

### Step D: 8-((benzyloxy)methyl)-9-(2,2-difluoroethyl)-3-methyl-3,9-dihydro-1H-purine-2,6-dione

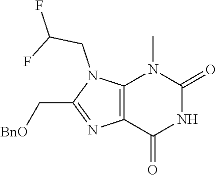 [SMILES: Cn1c(=O)[nH]c(=O)c2nc(COCc3ccccc3)n(CC(F)F)c21]

A mixture of 5-amino-6-((2,2-difluoroethyl)amino)-1-methylpyrimidine-2,4 (1H,3H)-dione (2.9 g, 13.2 mmol) in 2-(benzyloxy)acetic acid (2.52 g, 14.5 mmol) was stirred at 240° C. for 1.5 hour. The mixture was allowed cool to room temperature and dissolved in aq.NaOH (2.0 M, 20 mL), then acidified to pH=5˜6 with aq.HCl and filtered. The cake was collected, dried to give the titled compound (2.3 g, 50%). MS: M/e 351 (M+1)⁺.

### Step E: tert-butyl (2R,5S)-4-(8-((benzyloxy)methyl)-9-(2,2-difluoroethyl)-3-methyl-2-oxo-3,9-dihydro-2H-purin-6-yl)-2,5-dimethylpiperazine-1-carboxylate

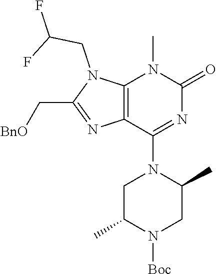 [SMILES: C[C@@H]1CN(c2nc(=O)n(C)c3c2nc(COCc2ccccc2)n3CC(F)F)[C@@H](C)CN1C(=O)OC(C)(C)C]

To a stirred mixture of 8-((benzyloxy)methyl)-9-(2,2-difluoroethyl)-3-methyl-3,9-dihydro-1H-purine-2,6-dione (1.05 mg, 3 mmol) and tert-butyl (2R,5S)-2,5-dimethylpiperazine-1-carboxylate (1.28 g, 6 mmol) in CH₃CN (15 mL) was added BOP (2.65 g, 6 mmol), followed by DBU (918 mg, 6 mmol). After the addition, the reaction mixture was concentrated to give the residue, which was dissolved in EtOAc (50 mL), washed with H₂O (20 mL), brine, dried over Na₂SO₄, concentrated and purified by flash column chromatography to give the titled compound (1.1 g, 67%). MS: M/e 547 (M+1)⁺.

### Step F: tert-butyl (2R,5S)-4-(9-(2,2-difluoroethyl)-8-(hydroxymethyl)-3-methyl-2-oxo-3,9-dihydro-2H-purin-6-yl)-2,5-dimethylpiperazine-1-carboxylate

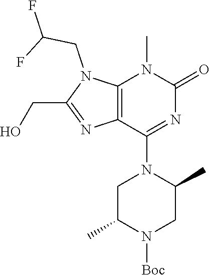 [SMILES: C[C@@H]1CN(c2nc(=O)n(C)c3c2nc(CO)n3CC(F)F)[C@@H](C)CN1C(=O)OC(C)(C)C]

To a stirred solution of tert-butyl (2R,5S)-4-(8-((benzyloxy)methyl)-9-(2,2-difluoroethyl)-3-methyl-2-oxo-3,9-dihydro-2H-purin-6-yl)-2,5-dimethylpiperazine-1-carboxylate (1.1 g, 2 mmol) in MeOH/AcOH (80 mL/2 mL) was added Pd/C (200 mg). After the addition, the reaction mixture was stirred over a weekend under H₂ (1 atm). The reaction mixture was filtered, and filtrate was concentrated to give the titled compound (crude, 100%). MS: M/e 457 (M+1)⁺.

### Step G: tert-butyl (2R,5S)-4-(8-(cyanomethyl)-9-(2,2-difluoroethyl)-3-methyl-2-oxo-3,9-dihydro-2H-purin-6-yl)-2,5-dimethylpiperazine-1-carboxylate

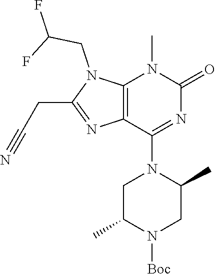 [SMILES: C[C@@H]1CN(c2nc(=O)n(C)c3c2nc(CC#N)n3CC(F)F)[C@@H](C)CN1C(=O)OC(C)(C)C]

To a stirred solution of tert-butyl (2R,5S)-4-(9-(2,2-difluoroethyl)-8-(hydroxymethyl)-3-methyl-2-oxo-3,9-dihydro-2H-purin-6-yl)-2,5-dimethylpiperazine-1-carboxylate (crude, 2 mmol) in CH₂Cl₂ (10 mL) was added SOCl₂ (0.26 g, 2.2 mmol). After stirred for 10 min, the reaction mixture was quenched with aq.NaHCO₃, washed with brine, dried over Na₂SO₄, concentrated to give the intermediate, dissolved in CH₂Cl₂ (10 mL), TBAF (1.0 M, 4 mL, 4 mmol) was added followed by TMSCN (0.4 g, 4 mmol). After the addition, the reaction mixture was stirred for 3 hours. The reaction mixture was concentrated to give the residue, treated with H₂O and extracted with EtOAc (15 mL×3). The combined organic layers were washed with brine, dried over Na₂SO₄, concentrated to dryness. The resulting residue was purified by flash column chromatography to give the titled compound (250 mg, 27%). MS: M/e 466 (M+1)⁺.

### Step H: 2-(9-(2,2-difluoroethyl)-6-((2S,5R)-2,5-dimethylpiperazin-1-yl)-3-methyl-2-oxo-3,9-dihydro-2H-purin-8-yl)acetonitrile

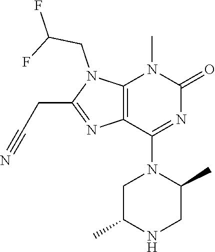 [SMILES: C[C@@H]1CN(c2nc(=O)n(C)c3c2nc(CC#N)n3CC(F)F)[C@@H](C)CN1]

To a stirred solution of tert-butyl (2R,5S)-4-(8-(cyanomethyl)-9-(2,2-difluoroethyl)-3-methyl-2-oxo-3,9-dihydro-2H-purin-6-yl)-2,5-dimethylpiperazine-1-carboxylate (250 mg, 0.54 mmol) in EtOAc (10 mL) was added dioxane/HCl (g) (4.0 M, 4 mL). After the addition, the reaction mixture was stirred for 4 hours. The reaction mixture was concentrated to give the residue, which was treated with CH₃CN/aq.NaHCO₃ (20 mL/3 mL), stirred for 10 min and filtered. The filtrate was concentrated, dried to give the titled compound (190 mg, 96%), which was directly used to the next step. MS: M/e 366 (M+1)⁺.

### Step I: 2-(9-(2,2-difluoroethyl)-6-((2S,5R)-4-(1-(3,3-dimethyl-2,3-dihydrobenzo [b][1,4]dioxin-6-yl)ethyl)-2,5-dimethylpiperazin-1-yl)-3-methyl-2-oxo-3,9-dihydro-2H-purin-8-yl)acetonitrile

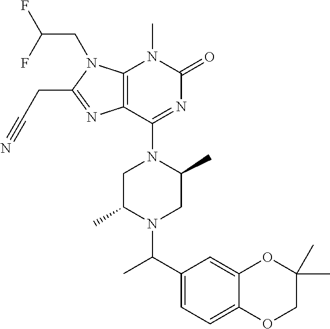 [SMILES: CC(c1ccc2c(c1)OC(C)(C)CO2)N1C[C@H](C)N(c2nc(=O)n(C)c3c2nc(CC#N)n3CC(F)F)C[C@H]1C]

A mixture of 2-(9-(2,2-difluoroethyl)-6-((2S,5R)-2,5-dimethylpiperazin-1-yl)-3-methyl-2-oxo-3,9-dihydro-2H-purin-8-yl)acetonitrile (190 mg, 0.52 mmol), 1-(3,3-dimethyl-2,3-dihydrobenzo[b][1,4]dioxin-6-yl)ethan-1-ol (324 mg, 1.56 mmol), (cyanomethyl)trimethylphosphonium iodide (505 mg, 2.08 mmol) and DIPEA (670 mg, 5.2 mmol) in CH₃CN (6 mL) was stirred overnight at 100° C. in a sealed tube. The reaction mixture was diluted with EtOAc (20 mL), washed with H₂O, brine, dried over Na₂SO₄, concentrated to give the residue, which was purified by Pre-TLC (CH₂Cl₂/MeOH=10:1) to give the desired compound, and further purified by Prep-HPLC (Method A) to give the titled compound (36 mg, 14%). ¹H NMR (400 MHz, CD₃OD) δ 6.90-6.75 (m, 3H), 6.33 (t, J=54.0 Hz, 1H), 6.01-5.04 (m, 2H), 4.93-4.85 (m, 2H), 4.24-4.16 (m, 1H), 3.88 (d, J=3.2 Hz, 2H), 3.72 (s, 3H), 3.66-3.38 (m, 3H), 3.08-2.68 (m, 2.5H), 2.33 (d, J=11.2 Hz, 0.5H), 1.51-1.40 (m, 1.5H), 1.37-1.23 (m, 10.5H), 1.11-0.91 (m, 3H) ppm. MS: M/e 556 (M+1)⁺.

### Compound A88: 2-(6-((2S,5R)-4-(1-(3,3-dimethyl-2,3-dihydrobenzo[b][1,4]dioxin-6-yl)ethyl)-2,5-dimethylpiperazin-1-yl)-3-methyl-2-oxo-9-propyl-3,9-dihydro-2H-purin-8-yl)acetonitrile

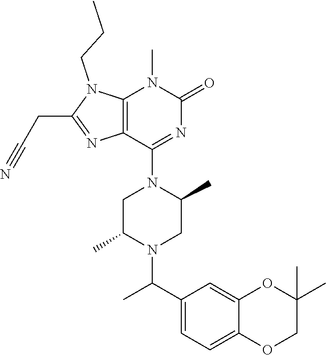 [SMILES: CCCn1c(CC#N)nc2c(N3C[C@@H](C)N(C(C)c4ccc5c(c4)OC(C)(C)CO5)C[C@@H]3C)nc(=O)n(C)c21]

### Step A: 1-methyl-6-(propylamino)pyrimidine-2,4 (1H,3H)-dione

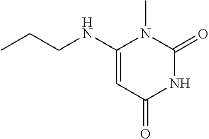

A solution of 6-chloro-1-methylpyrimidine-2,4 (1H,3H)-dione (5 g, 31.25 mmol) and propan-1-amine (3.7 g, 62.71 mmol) in EtOH (50 ml) was stirred at 60° C. for 4 h. The mixture was filtered to give the titled compound (4.6 g, 80%). MS: M/e 184 (M+1)⁺.

### Step B: 1-methyl-5-nitroso-6-(propylamino)pyrimidine-2,4 (1H,3H)-dione

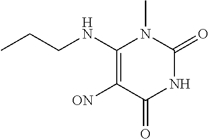

To a solution of 1-methyl-6-(propylamino)pyrimidine-2,4 (1H,3H)-dione (4.59 g, 25.08 mmol) in H₂O (60 ml), NaNO₂ (1.90 g, 27.54 mmol) was added at 0˜5° C. Then AcOH (4.51 g, 75.17 mmol) was added dropwise to the above solution and stirred at 0˜5° C. for 15 min. The reaction mixture was filtered to give the titled compound (5 g, 94%). MS: M/e 213 (M+1)⁺.

### Step C: 5-amino-1-methyl-6-(propylamino)pyrimidine-2,4 (1H,3H)-dione

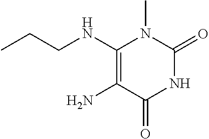

A solution of 1-methyl-5-nitroso-6-(propylamino)pyrimidine-2,4 (1H,3H)-dione (5 g, 23.58 mmol) and Na₂S₂O₄ (12 g, 68.97 mmol) in H₂O (50 ml) was stirred at RT for 30 min. The reaction mixture was filtered and dried to give the titled compound (3.8 g, 81%). MS: M/e 199 (M+1)⁺.

### Step D: 8-((benzyloxy)methyl)-3-methyl-9-propyl-3,9-dihydro-1H-purine-2,6-dione

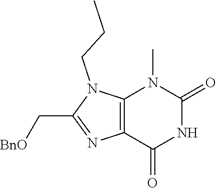 [SMILES: CCCn1c(COCc2ccccc2)nc2c(=O)[nH]c(=O)n(C)c21]

5-amino-1-methyl-6-(propylamino)pyrimidine-2,4 (1H,3H)-dione (3.8 g, 19.19 mmol) and 2-(benzyloxy)acetic acid (3.5 g, 21.08 mmol) were stirred at 240° C. for 2 hrs. The mixture was cooled to RT, and then dissolved in aq. NaOH (2N, 20 ml). The solution was neutralized with aq. HCl (2N) to pH=7, which was then filtered. The resulting solid was dried to give the titled compound (3.7 g, 59%). MS: M/e 329 (M+1)⁺.

### Step E: tert-butyl (2R,5S)-4-(8-((benzyloxy)methyl)-3-methyl-2-oxo-9-propyl-3,9-dihydro-2H-purin-6-yl)-2,5-dimethylpiperazine-1-carboxylate

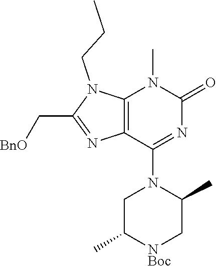 [SMILES: CCCn1c(COCc2ccccc2)nc2c(N3C[C@@H](C)N(C(=O)OC(C)(C)C)C[C@@H]3C)nc(=O)n(C)c21]

A solution of 8-((benzyloxy)methyl)-3-methyl-9-propyl-3,9-dihydro-1H-purine-2,6-dione (3.2 g, 9.76 mmol), tert-butyl (2R,5S)-2,5-dimethylpiperazine-1-carboxylate (2.3 g, 10.75 mmol), BOP (6.5 g, 14.71 mmol) and DBU (4.5 g, 29.41 mmol) in MeCN (35 ml) was stirred at RT overnight. The reaction solution was dissolved in EtOAc (50 ml) and washed with brine (20 ml×2). The organic layer was dried over Na₂SO₄ and concentrated under reduced pressure. The residue was purified by flash column chromatography with 0-5% MeOH in DCM to give the titled compound (4 g, 78%). MS: M/e 525 (M+1)⁺

### Step F: 6-((2S,5R)-2,5-dimethylpiperazin-1-yl)-8-(hydroxymethyl)-3-methyl-9-propyl-3,9-dihydro-2H-purin-2-one

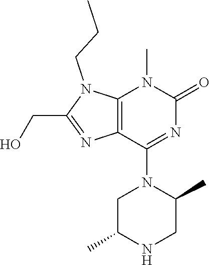 [SMILES: CCCn1c(CO)nc2c(N3C[C@@H](C)NC[C@@H]3C)nc(=O)n(C)c21]

To a solution of tert-butyl (2R,5S)-4-(8-((benzyloxy)methyl)-3-methyl-2-oxo-9-propyl-3,9-dihydro-2H-purin-6-yl)-2,5-dimethylpiperazine-1-carboxylate (4 g, 7.63 mmol) in DCM (50 ml) at RT, was added BBr₃ (1M in DCM, 22.90 ml, 22.90 mmol) dropwise. The solution was stirred at RT for 6 hrs and then evaporated to dryness. The resulting residue was slurried in DCM & MeOH and then filtered. The filtrate was evaporated to dryness to give the titled compound (2.5 g, crude), which was used directly for the next step without further purification. MS: M/e 335 (M+1)⁺.

### Step G: tert-butyl (2R,5S)-4-(8-(hydroxymethyl)-3-methyl-2-oxo-9-propyl-3,9-dihydro-2H-purin-6-yl)-2,5-dimethylpiperazine-1-carboxylate

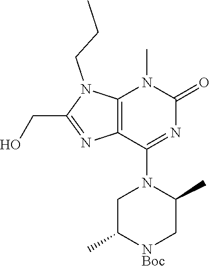 [SMILES: CCCn1c(CO)nc2c(N3C[C@@H](C)N(C(=O)OC(C)(C)C)C[C@@H]3C)nc(=O)n(C)c21]

A solution of 6-((2S,5R)-2,5-dimethylpiperazin-1-yl)-8-(hydroxymethyl)-3-methyl-9-propyl-3,9-dihydro-2H-purin-2-one (2.5 g, crude), (Boc)₂O (1.79 g, 8.21 mmol) and TEA (1.5 g, 14.85 mmol) in THE (30 ml) and H₂O (2 ml) was stirred at RT overnight. The mixture was diluted with EtOAc (30 ml) and then washed with brine (20 ml×2). The organic layer was dried over Na₂SO₄ and concentrated under reduced pressure. The residue was purified by flash column chromatography with 0-5% MeOH in DCM to give the titled compound (1.05 g). MS: M/e 435 (M+1)⁺.

### Step H: tert-butyl (2R,5S)-4-(8-(chloromethyl)-3-methyl-2-oxo-9-propyl-3,9-dihydro-2H-purin-6-yl)-2,5-dimethylpiperazine-1-carboxylate

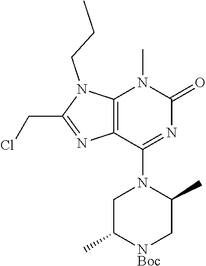 [SMILES: CCCn1c(CCl)nc2c(N3C[C@@H](C)N(C(=O)OC(C)(C)C)C[C@@H]3C)nc(=O)n(C)c21]

A solution of tert-butyl (2R,5S)-4-(8-(hydroxymethyl)-3-methyl-2-oxo-9-propyl-3,9-dihydro-2H-purin-6-yl)-2,5-dimethylpiperazine-1-carboxylate (500 mg, 1.15 mmol) and SOCl₂ (411 mg, 3.45 mmol) in DCM (10 ml) was stirred at 0° C. for 15 min. The solution was washed with aq. NaHCO₃ (10 ml), dried over Na₂SO₄ and evaporated to dryness to give the titled compound (520 mg, 100%), which was used directly for the next step.

### Step I: tert-butyl (2R,5S)-4-(8-(cyanomethyl)-3-methyl-2-oxo-9-propyl-3,9-dihydro-2H-purin-6-yl)-2,5-dimethylpiperazine-1-carboxylate

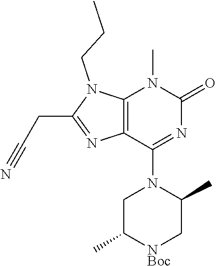 [SMILES: CCCn1c(CC#N)nc2c(N3C[C@@H](C)N(C(=O)OC(C)(C)C)C[C@@H]3C)nc(=O)n(C)c21]

A solution of tert-butyl (2R,5S)-4-(8-(chloromethyl)-3-methyl-2-oxo-9-propyl-3,9-dihydro-2H-purin-6-yl)-2,5-dimethylpiperazine-1-carboxylate (520 mg, 1.15 mmol), trimethylsilanecarbonitrile (342 mg, 3.45 mmol) and Cs₂CO₃ (1.1 g, 3.37 mmol) in MeCN (10 ml) was stirred at 70° C. overnight. The mixture was diluted with EtOAc (20 ml) and washed with brine (15 ml). The organic layer was dried over Na₂SO₄ and concentrated. The resulting residue was purified by flash column chromatography with 0-5% MeOH in DCM to give the titled compound (210 mg, 41%). MS: M/e 444 (M+1)⁺.

### Step J: 2-(6-((2S,5R)-2,5-dimethylpiperazin-1-yl)-3-methyl-2-oxo-9-propyl-3,9-dihydro-2H-purin-8-yl)acetonitrile

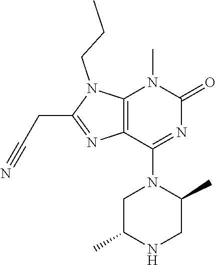 [SMILES: CCCn1c(CC#N)nc2c(N3C[C@@H](C)NC[C@@H]3C)nc(=O)n(C)c21]

A solution of tert-butyl (2R,5S)-4-(8-(cyanomethyl)-3-methyl-2-oxo-9-propyl-3,9-dihydro-2H-purin-6-yl)-2,5-dimethylpiperazine-1-carboxylate (210 mg, 0.47 mmol) and TFA (2 ml) in DCM (10 ml) was stirred at RT for 4 hrs. The solution was washed with aq. NaHCO₃, dried over Na₂SO₄ and evaporated to dryness to give the titled compound (162.5 mg, 100%), which was used directly for the next step without further purification. MS: M/e 344 (M+1)⁺.

### Step K: 2-(6-((2S,5R)-4-(1-(3,3-dimethyl-2,3-dihydrobenzo[b][1,4]dioxin-6-yl)ethyl)-2,5-dimethylpiperazin-1-yl)-3-methyl-2-oxo-9-propyl-3,9-dihydro-2H-purin-8-yl)acetonitrile

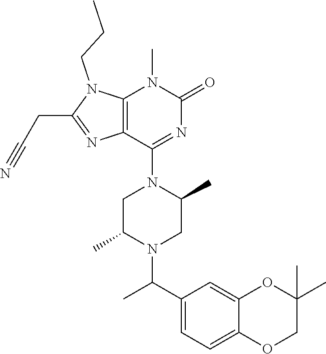 [SMILES: CCCn1c(CC#N)nc2c(N3C[C@@H](C)N(C(C)c4ccc5c(c4)OC(C)(C)CO5)C[C@@H]3C)nc(=O)n(C)c21]

A solution of 2-(6-((2S,5R)-2,5-dimethylpiperazin-1-yl)-3-methyl-2-oxo-9-propyl-3,9-dihydro-2H-purin-8-yl)acetonitrile (160 mg, 0.47 mmol), 1-(3,3-dimethyl-2,3-dihydrobenzo[b][1,4]dioxin-6-yl)ethan-1-ol (291 mg, 1.40 mmol), (cyanomethyl)trimethylphosphonium iodide (340 mg, 1.40 mmol) and DIPEA (602 mg, 4.67 mmol) in MeCN (4 ml) was stirred at 100° C. overnight. The reaction was diluted with EtOAc (10 ml) and washed with brine (10 ml). The organic layer was concentrated under reduced pressure. The resulting residue was purified by flash column chromatography with 0-5% MeOH in DCM and then Prep-HPLC (Method A) to give the titled compound (47.1 mg). ¹H NMR (400 MHz, DMSO-d6) δ 6.86-6.77 (m, 3H), 6.10-5.36 (m, 1H), 5.20-4.52 (m, 1H), 4.40 (s, 2H), 4.13 (s, 2H), 3.90 (d, J=4.5 Hz, 2H), 3.59 (s, 3H), 3.45 (q, J=6.1 Hz, 1.5H), 3.32 (d, J=6.4 Hz, 2H), 2.76 (d, J=36.8 Hz, 1H), 2.18 (d, J=10.8 Hz, 0.5H), 1.72 (q, J=7.4 Hz, 2H), 1.40-1.24 (m, 7.5H), 1.23-1.13 (m, 4.5H), 0.91 (t, J=7.0 Hz, 4.5H), 0.80 (d, J=6.2 Hz, 1.5H) ppm. MS. M/e 534 (M+1)⁺.

### Compound A91: 2-(6-((2S,5R)-2,5-dimethyl-4-(1-(3-methyl-4-oxo-4H-chromen-6-yl)ethyl)piperazin-1-yl)-9-ethyl-3-methyl-2-oxo-3,9-dihydro-2H-purin-8-yl)acetonitrile

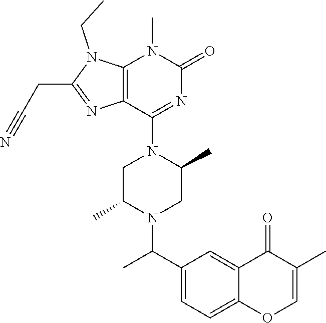 [SMILES: CCn1c(CC#N)nc2c(N3C[C@@H](C)N(C(C)c4ccc5occ(C)c(=O)c5c4)C[C@@H]3C)nc(=O)n(C)c21]

### Step A: 6-bromo-3-methyl-4H-chromen-4-one

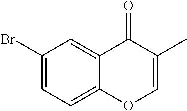

To a solution of 1-(5-bromo-2-hydroxyphenyl)propan-1-one (600 mg, 2.6 mmol) in DMF (10 mL) was added DMF-DMA (626 mg, 5.2 mol). The mixture was stirred at 120° C. for 3 hrs. The organic layer was diluted with EtOAc and washed with brine, dried over MgSO₄, filtered and the solvent was removed under vacuum. The resulting residue was purified by flash column chromatography (PE:EA) to give the titled compound (510 mg, 88%). MS: M/e 221 (M+1-18)⁺.

### Step B: 6-acetyl-3-methyl-4H-chromen-4-one

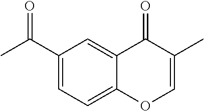

To a solution of 6-bromo-3-methyl-4H-chromen-4-one (400 mg, 1.7 mmol) in toluene (10 mL) was added 1-(vinyloxy) butane (1.7 g, 3.5 mmol), Pd(PPh₃)₂Cl₂ (120 mg, 0.17 mmol). The mixture was stirred at 100° C. under N₂ for 16 hrs. The reaction was cooled to room temperature, treated with dioxane (4N HCl, 1 mL) dropwise, stirred at room temperature for one hour. The mixture was concentrated to give titled compound for the next step.

### Step C: 6-(1-hydroxyethyl)-3-methyl-4H-chromen-4-one

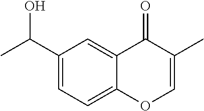

To a solution of 6-acetyl-3-methyl-4H-chromen-4-one (crude, 1.7 mmol) in MeOH (2 ml) at 0° C. was added NaBH₄ (65 mg, 1.7 mmol) in some portions. After addition, the reaction was stirred at 0° C. for 5 min. The reaction was quenched by adding water and extracted with DCM, washed with brine, dried over Na₂SO₄, filtered, concentrated to give the titled compound (90 mg, two steps 27%). MS: M/e 187 (M+1-18)⁺.

### Step D: 2-(6-((2S,5R)-2,5-dimethyl-4-(1-(3-methyl-4-oxo-4H-chromen-6-yl)ethyl)piperazin-1-yl)-9-ethyl-3-methyl-2-oxo-3,9-dihydro-2H-purin-8-yl)acetonitrile

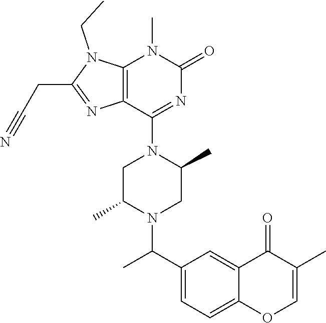 [SMILES: CCn1c(CC#N)nc2c(N3C[C@@H](C)N(C(C)c4ccc5occ(C)c(=O)c5c4)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (80 mg, 0.24 mol), 6-(1-hydroxyethyl)-3-methyl-4H-chromen-4-one (90 mg, 0.48 mol), (cyanomethyl)trimethylphosphonium iodide (175 mg, 0.72 mol) and DIPEA (310 mg, 2.4 mol) in CH₃CN (2 ml). Then the mixture solution was stirred at 105° C. for 16 hours. The reaction mixture was concentrated under reduced pressure, then the crude was diluted with DCM and washed with brine, dried over MgSO₄, filtered and the solvent was removed under vacuum. The resulting residue was purified by flash column chromatography (DCM:MeOH) to give the titled compound (42 mg). ¹H NMR (400 MHz, CD₃OD) δ 8.17-8.07 (m, 2H), 7.87 (t, J=8.0 Hz, 1H), 7.56 (dd, J=12.5, 8.8 Hz, 1H), 6.15-4.91 (m, 2H), 4.42-4.31 (m, 2H), 3.79 (d, J=6.8 Hz, 1H), 3.76 (s, 3H), 3.70-3.40 (m, 2H), 3.28-3.15 (m, 1H), 2.99-2.80 (m, 2H), 2.79-2.10 (m, 1H), 2.02 (s, 3H), 1.50-1.43 (m, 4H), 1.41-1.22 (m, 5H), 1.10-0.87 (m, 3H) ppm. MS: M/e 516 (M+1)⁺.

### Compound A94: 2-(6-((2S,5R)-4-(1-(2,2-dimethylchroman-7-yl)ethyl)-2,5-dimethylpiperazin-1-yl)-9-ethyl-3-methyl-2-oxo-3,9-dihydro-2H-purin-8-yl)acetonitrile

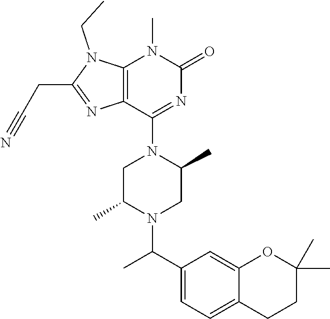 [SMILES: CCn1c(CC#N)nc2c(N3C[C@@H](C)N(C(C)c4ccc5c(c4)OC(C)(C)CC5)C[C@@H]3C)nc(=O)n(C)c21]

### Step A: 7-bromo-2,2-dimethylchroman-4-one

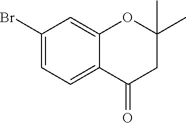

To a solution of 1-(4-bromo-2-hydroxyphenyl)ethan-1-one (4 g, 0.019 mol) in Tol. (16 ml) was added pyrrolidine (4 ml) at 0˜5° C. and stirred for 20 min. Acetone (1.62 g, 0.030 mmol) was added to the above solution and stirred at 80° C. overnight. The solution was diluted with EtOAc (20 ml), washed with brine (10 ml), dried and concentrated. The residue was purified by flash column chromatography with 20%-40% DCM in PE to give the titled compound (3.6 g, 75%). ¹H NMR (400 MHz, CDCl₃) δ 7.71 (d, J=8.3 Hz, 1H), 7.14 (s, 1H), 7.11 (d, J=9.6 Hz, 1H), 2.71 (s, 2H), 1.45 (s, 6H) ppm.

### Step B: 7-bromo-2,2-dimethylchroman-4-ol

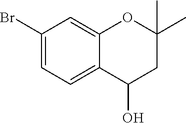

A solution of 7-bromo-2,2-dimethylchroman-4-one (3.1 g, 0.012 mol) and NaBH₄ (0.46 g, 0.012 mol) in MeOH (30 ml) was stirred at RT for 15 min. The solution was quenched with water (10 ml) and then concentrated under reduced pressure. The residue was diluted with EtOAc (30 ml), washed with brine (10 ml), dried and evaporated to dryness to give the titled compound (3.1 g, 100%), which was used directly for the next step. MS: M/e 257 (M+1)⁺.

### Step C: 7-bromo-2,2-dimethylchromane

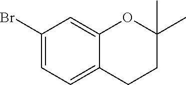

A solution of 7-bromo-2,2-dimethylchroman-4-ol (3.1 g, 0.012 mol) in TFA (10 ml) and triethylsilane (10 ml) was stirred at RT for 30 min. The solution was diluted with EtOAc (30 ml), washed with brine (15 ml), died and then concentrated. The residue was purified by flash column chromatography with 0%-5% EtOAc in PE to give the titled compound (2.1 g, 72%). ¹H NMR (400 MHz, CDCl₃) δ 6.97-6.87 (m, 3H), 2.71 (t, J=6.8 Hz, 2H), 1.79 (t, J=6.8 Hz, 2H), 1.32 (s, 6H) ppm.

### Step D: 1-(2,2-dimethylchroman-7-yl)ethan-1-one

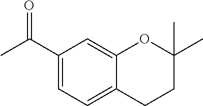

A solution of 7-bromo-2,2-dimethylchromane (500 mg, 2.07 mmol), Pd(PPh₃)₂Cl₂ (73 mg, 0.10 mmol) and tributyl(1-ethoxyvinyl)stannane (1.1 g, 3.05 mmol) in Tol. (10 ml) was stirred at 100° C. overnight. The mixture was cooled to RT and HCl (4M in dioxane, 2 ml) was added, stirred for 30 min. The mixture was diluted with EtOAc (20 ml), washed with brine (10 ml), dried and concentrated. The residue was purified by flash column chromatography with 0%-10% EtOAc in PE to give the titled compound (250 mg, 59%). ¹H NMR (400 MHz, CDCl₃) δ 7.43 (d, J=8.0 Hz, 1H), 7.36 (s, 1H), 7.14 (d, J=8.0 Hz, 1H), 2.82 (t, J=6.7 Hz, 2H), 2.54 (s, 3H), 1.84 (t, J=6.7 Hz, 2H), 1.35 (s, 6H) ppm.

### Step E: 1-(2,2-dimethylchroman-7-yl)ethan-1-ol

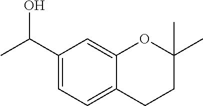

A solution of 1-(2,2-dimethylchroman-7-yl)ethan-1-one (250 mg, 1.23 mmol) and NaBH₄ (47 mg, 1.24 mmol) in MeOH (4 ml) and THE (1 ml) was stirred at RT for 30 min. The solution was quenched with water (10 ml) and then concentrated under reduced pressure. The residue was diluted with EtOAc (15 ml), washed with brine (10 ml), dried and evaporated to dryness to give the titled compound (250 mg, 100%), which was used directly for the next step. MS: M/e 207 (M+1)⁺.

### Step F: 2-(6-((2S,5R)-4-(1-(2,2-dimethylchroman-7-yl)ethyl)-2,5-dimethylpiperazin-1-yl)-9-ethyl-3-methyl-2-oxo-3,9-dihydro-2H-purin-8-yl)acetonitrile

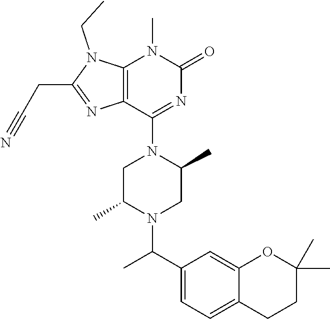 [SMILES: CCn1c(CC#N)nc2c(N3C[C@@H](C)N(C(C)c4ccc5c(c4)OC(C)(C)CC5)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (200 mg, 0.61 mmol), 1-(2,2-dimethylchroman-7-yl)ethan-1-ol (250 mg, 1.21 mmol), (cyanomethyl)trimethylphosphonium iodide (443 mg, 1.82 mmol) and DIPEA (784 mg, 6.08 mmol) in MeCN (3 ml) was stirred at 100° C. overnight. The reaction was diluted with EtOAc (10 ml) and washed with brine (10 ml). The organic layer was concentrated under reduced pressure. The resulting residue was purified by flash column chromatography with 0-5% MeOH in DCM to give the titled Compound A94 (70 mg), which was further separated into Compound A94a (21 mg) and Compound A94b (21 mg) by chiral Prep-HPLC. The chiral separation conditions are shown below.

| | |
|---|---|
| Column | CHIRALPAK IH |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A94: ¹H NMR (400 MHz, DMSO-d6) δ 7.01 (t, J=7.6 Hz, 1H), 6.81 (d, J=7.6 Hz, 1H), 6.68 (s, 1H), 6.10-5.40 (m, 1H), 5.20-4.50 (m, 1H), 4.40 (s, 2H), 4.26 (d, J=6.4 Hz, 2H), 3.61 (s, 3H), 3.45 (s, 1H), 3.33 (s, 1.5H), 2.70 (q, J=5.3 Hz, 3H), 2.44 (s, 1H), 2.17 (d, J=12.3 Hz, 0.5H), 1.75 (q, J=4.2 Hz, 2H), 1.39-1.12 (m, 15H), 0.93 (s, 1.5H), 0.80 (d, J=6.0 Hz, 1.5H) ppm. MS: M/e 518 (M+1)⁺.

Compound A94a (the earlier peak): ¹H NMR (400 MHz, DMSO-d6) δ 7.02 (d, J=7.3 Hz, 1H), 6.81 (d, J=7.5 Hz, 1H), 6.68 (s, 1H), 6.12-5.39 (m, 1H), 5.20-4.42 (m, 1H), 4.39 (s, 2H), 4.25 (s, 2H), 3.60 (s, 3H), 3.43 (s, 1H), 3.33 (s, 2H), 2.77-2.65 (m, 4H), 1.75 (t, J=7.8 Hz, 2H), 1.33 (s, 6H), 1.27 (s, 6H), 1.19 (d, J=5.5 Hz, 3H), 0.80 (d, J=5.1 Hz, 3H) ppm. MS: M/e 518 (M+1)⁺.

Compound A94b (the later peak): ¹H NMR (400 MHz, DMSO-d6) δ 7.00 (d, J=7.1 Hz, 1H), 6.82 (d, J=7.3 Hz, 1H), 6.68 (s, 1H), 5.95-5.60 (m, 1H), 5.00-4.55 (m, 1H), 4.40 (s, 2H), 4.27 (s, 2H), 3.61 (s, 3H), 3.46 (s, 1H), 3.33 (s, 3H), 2.70 (t, J=4.0 Hz, 2H), 2.18 (d, J=7.8 Hz, 1H), 1.74 (t, J=7.8 Hz, 2H), 1.34 (t, J=6.8 Hz, 3H), 1.26 (d, J=4.3 Hz, 6H), 1.21 (d, J=5.8 Hz, 3H), 1.17 (s, 3H), 0.93 (s, 3H) ppm. MS: M/e 518 (M+1)⁺.

### Compound A95: 2-(9-ethyl-6-((2S,5R)-4-(1-(2-ethylbenzo[d]thiazol-5-yl)ethyl)-2,5-dimethylpiperazin-1-yl)-3-methyl-2-oxo-3,9-dihydro-2H-purin-8-yl)acetonitrile

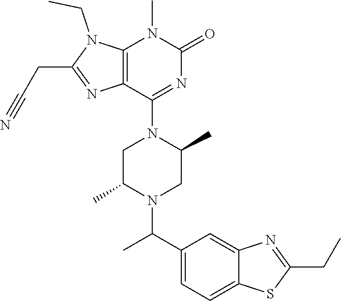 [SMILES: CCc1nc2cc(C(C)N3C[C@H](C)N(c4nc(=O)n(C)c5c4nc(CC#N)n5CC)C[C@H]3C)ccc2s1]

### Step A: 5-bromo-2-ethylbenzo[d]thiazole

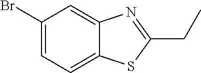

To a stirred solution of 5-bromo-2-methylbenzo[d]thiazole (1.3 g, 5.7 mmol) in THE (30 mL) was added dropwise LDA (2.0 M, 3.4 mL, 6.8 mmol) at −78° C. After stirred for 30 min, Mel (4.05 g, 28.5 mmol) was added dropwise at −78° C. The reaction mixture was quenched with aq.NH₄Cl, extracted with EtOAc (15 mL×3). The combined organic layers were washed with brine, dried over Na₂SO₄, concentrated and purified by flash column chromatography to give the titled compound (700 mg, 51%). MS: M/e 243 (M+1)⁺.

### Step B: 1-(2-ethylbenzo[d]thiazol-5-yl)ethan-1-one

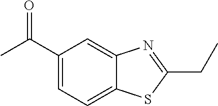

A mixture of 5-bromo-2-ethylbenzo[d]thiazole (400 mg, 1.65 mmol), tributyl(1-ethoxyvinyl)stannane (893 mg, 2.48 mmol) and Pd(PPh₃)₂Cl₂ (58 mg, 0.083 mmol) in toluene (10 mL) was stirred at 100° C. overnight under N₂. The reaction mixture was treated with aq.HCl (2.0 M, 5 mL) and stirred for 10 min, then extracted with EtOAc (15 mL×3). The combined organic layers were washed with brine, dried over Na₂SO₄, concentrated and purified by flash column chromatography to give the titled compound (250 mg, 74%). MS: M/e 206 (M+1)⁺.

### Step C: 1-(2-ethylbenzo[d]thiazol-5-yl)ethan-1-ol

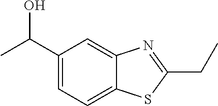

To a stirred solution of 1-(2-ethylbenzo[d]thiazol-5-yl)ethan-1-one (250 mg, 1.22 mmol) in MeOH (5 mL) was added NaBH₄ (46.36 mg, 1.22 mmol). After then, the mixture was stirred for 10 min. The reaction mixture was poured into H₂O (10 mL) and extracted with EtOAc (10 mL×3). The combined organic layers were washed with brine, dried over Na₂SO₄, concentrated to give the titled compound (240 mg, 95%). MS: M/e 208 (M+1)⁺.

### Step D: 2-(9-ethyl-6-((2S,5R)-4-(1-(2-ethylbenzo[d]thiazol-5-yl)ethyl)-2,5-dimethylpiperazin-1-yl)-3-methyl-2-oxo-3,9-dihydro-2H-purin-8-yl)acetonitrile

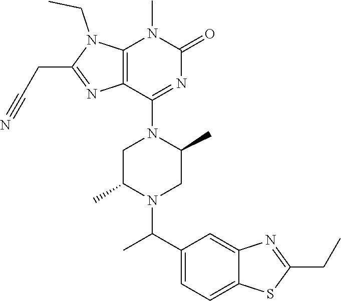 [SMILES: CCc1nc2cc(C(C)N3C[C@H](C)N(c4nc(=O)n(C)c5c4nc(CC#N)n5CC)C[C@H]3C)ccc2s1]

A mixture of 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol), 1-(2-ethylbenzo[d]thiazol-5-yl)ethan-1-ol (94 mg, 0.45 mmol), (cyanomethyl)trimethylphosphonium iodide (218 mg, 0.9 mmol) and DIPEA (387 mg, 3 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to give the residue, which was purified by Prep-HPLC (Method A) to give the titled Compound A95 (40 mg). which was further separated into Compound A95a (14 mg) and Compound A95b (15 mg) by chiral Prep-HPLC. The chiral separation conditions are shown below.

| | |
|---|---|
| Column | CHIRALPAK ID |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A95: ¹H NMR (400 MHz, CD₃OD) δ 7.97-7.87 (m, 2H), 7.50 (t, J=7.6 Hz, 1H), 6.25-4.94 (m, 2H), 4.43-4.31 (m, 2H), 4.27-3.80 (m, 2H), 3.76 (s, 3H), 3.73-3.64 (m, 1H), 3.34-3.31 (m, 2H), 3.21-3.12 (m, 2H), 3.00-2.91 (m, 1.5H), 2.81-2.68 (m, 0.5H), 1.53-1.23 (m, 12H), 1.12-0.92 (m, 3H) ppm. MS: M/e 519 (M+1)⁺.

Compound A95a (the earlier peak): ¹H NMR (400 MHz, CD₃OD) δ 7.93 (s, 1H), 7.91 (d, J=8.4 Hz, 1H), 7.50 (d, J=7.6 Hz, 1H), 6.35-5.04 (m, 2H), 4.36 (dd, J=13.6, 6.4 Hz, 2H), 4.26-4.16 (m, 1H), 3.82-3.77 (m, 1H), 3.76 (s, 3H), 3.65-3.40 (m, 1H), 3.32-3.29 (m, 2H), 3.16 (q, J=7.6 Hz, 2H), 2.99-2.82 (m, 2H), 1.56-1.41 (m, 9H), 1.37 (d, J=6.4 Hz, 3H), 0.92 (d, J=6.4 Hz, 3H)) ppm. MS: M/e 519 (M+1)⁺.

Compound A95b (the later peak): ¹H NMR (400 MHz, CD₃OD) δ 7.93 (s, 1H), 7.88 (d, J=8.4 Hz, 1H), 7.49 (d, J=8.4 Hz, 1H), 6.11-4.94 (m, 2H), 4.37 (q, J=7.2 Hz, 2H), 4.26-4.16 (m, 1H), 3.76 (s, 3H), 3.70-3.58 (m, 2H), 3.34-3.31 (m, 2H), 3.15 (q, J=7.6 Hz, 2H), 2.78-2.59 (m, 1H), 2.23 (d, J=12 Hz, 1H), 1.52-1.37 (m, 9H), 1.27 (d, J=6.4 Hz, 3H), 1.08 (d, J=6.4 Hz, 3H) ppm. MS: M/e 519 (M+1)⁺.

### Compound A96: 2-(9-ethyl-6-((2S,5R)-4-(1-(2-isopropylbenzo[d]thiazol-5-yl)ethyl)-2,5-dimethylpiperazin-1-yl)-3-methyl-2-oxo-3,9-dihydro-2H-purin-8-yl)acetonitrile

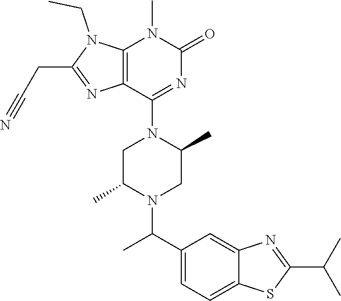 [SMILES: CCn1c(CC#N)nc2c(N3C[C@@H](C)N(C(C)c4ccc5sc(C(C)C)nc5c4)C[C@@H]3C)nc(=O)n(C)c21]

### Step A: 5-bromo-2-isopropylbenzo[d]thiazole

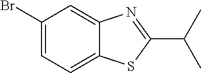

To a stirred solution of 5-bromo-2-methylbenzo[d]thiazole (2.28 g, 10 mmol) in THE (30 mL) was added dropwise LDA (2.0 M, 11 mL, 22 mmol) at −78° C. After stirred for 30 min, Mel (7.1 g, 50 mmol) was added dropwise at −78° C. The reaction mixture was quenched with aq.NH₄Cl, extracted with EtOAc (15 mL×3). The combined organic layers were washed with brine, dried over Na₂SO₄, concentrated and purified by flash column chromatography to give the titled compound (1.5 g, 59%). MS: M/e 257 (M+1)⁺.

### Step B: 1-(2-isopropylbenzo[d]thiazol-5-yl)ethan-1-one

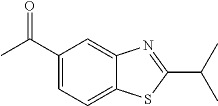

A mixture of 5-bromo-2-isopropylbenzo[d]thiazole (512 mg, 2 mmol), tributyl(1-ethoxyvinyl)stannane (1.08 g, 3 mmol) and Pd(PPh₃)₂Cl₂ (70 mg, 0.1 mmol) in toluene (10 mL) was stirred at 100° C. overnight under N₂. The reaction mixture was treated with aq.HCl (2.0 M, 5 mL) and stirred for 10 min, then extracted with EtOAc (15 mL×3). The combined organic layers were washed with brine, dried over Na₂SO₄, concentrated and purified by flash column chromatography to give the titled compound (385 mg, 88%). MS: M/e 220 (M+1)⁺.

### Step C: 1-(2-isopropylbenzo[d]thiazol-5-yl)ethan-1-ol

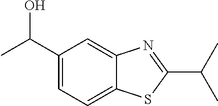

To a stirred solution of 1-(2-isopropylbenzo[d]thiazol-5-yl)ethan-1-one (385 mg, 1.76 mmol) in MeOH (5 mL) was added NaBH₄ (67 mg, 1.76 mmol). After then, the mixture was stirred for 10 min. The reaction mixture was poured into H₂O (10 mL) and extracted with EtOAc (10 mL×3). The combined organic layers were washed with brine, dried over Na₂SO₄, concentrated to give the titled compound (212 mg, 55%). MS: M/e 222 (M+1)⁺.

### Step D: 2-(9-ethyl-6-((2S,5R)-4-(1-(2-isopropylbenzo[d]thiazol-5-yl)ethyl)-2,5-dimethyl piperazin-1-yl)-3-methyl-2-oxo-3,9-dihydro-2H-purin-8-yl)acetonitrile

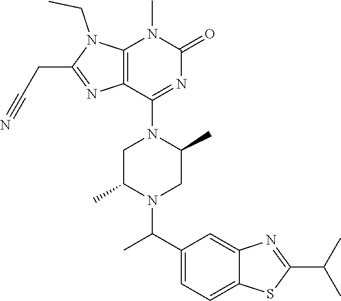 [SMILES: CCn1c(CC#N)nc2c(N3C[C@@H](C)N(C(C)c4ccc5sc(C(C)C)nc5c4)C[C@@H]3C)nc(=O)n(C)c21]

A mixture of 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol), 1-(2-isopropylbenzo[d]thiazol-5-yl)ethan-1-ol (99.4 mg, 0.45 mmol), (cyanomethyl)trimethylphosphonium iodide (218 mg, 0.9 mmol) and DIPEA (387 mg, 3 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to give the residue, which was purified by Prep-HPLC (Method A) to give the titled compound (55 mg). ¹H NMR (400 MHz, CD₃OD) δ 8.00-7.87 (m, 2H), 7.54-7.45 (m, 1H), 6.34-4.97 (m, 2H), 4.46-4.31 (m, 2H), 4.29-3.82 (m, 2H), 3.76 (s, 3H), 3.73-3.63 (m, 1H), 3.48-3.37 (m, 1H), 3.04-2.64 (m, 4H), 1.54-1.24 (m, 15H), 1.03 (dd, J=56.4, 6.0 Hz, 3H) ppm. MS: M/e 533 (M+1)⁺.

### Compound A97: 2-(6-((2S,5R)-4-(1-(2,2-dimethyl-2,3-dihydrobenzofuran-6-yl)ethyl)-2,5-dimethylpiperazin-1-yl)-9-ethyl-3-methyl-2-oxo-3,9-dihydro-2H-purin-8-yl)acetonitrile

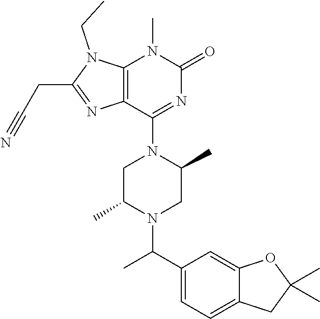 [SMILES: CCn1c(CC#N)nc2c(N3C[C@@H](C)N(C(C)c4ccc5c(c4)OC(C)(C)C5)C[C@@H]3C)nc(=O)n(C)c21]

### Step A: 1-(4-bromo-2-fluorophenyl)-2-methylpropan-2-ol

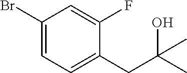

To a solution of methyl 2-(4-bromo-2-fluorophenyl)acetate (1 g, 4.05 mmol) in THE (10 ml) at −70° C. under N₂, was added MeMgBr (3M, 3.37 ml, 10.11 mmol) dropwise. The solution was warmed to RT and then stirred at RT for 2 hrs. The reaction was quenched with brine (10 ml) and extracted with EtOAc (10 ml×2). The organic layer was concentrated under reduced pressure. The resulting residue was purified by flash column chromatography with 0-20% EtOAc in PE to give the titled compound (840 mg, 84%). ¹H NMR (400 MHz, CDCl₃) δ 7.24 (d, J=8.4 Hz, 2H), 7.14 (t, J=8.0 Hz, 1H), 2.78 (s, 2H), 1.24 (s, 6H) ppm.

### Step B: 6-bromo-2,2-dimethyl-2,3-dihydrobenzofuran

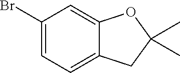

A solution of 1-(4-bromo-2-fluorophenyl)-2-methylpropan-2-ol (740 mg, 3.00 mmol) and tBuOK (1M, 3.5 ml, 3.5 mmol) in THE (10 ml) was stirred at 60° C. overnight. The reaction was diluted with EtOAc (20 ml) and washed with brine (10 ml). The organic layer was concentrated under reduced pressure. The resulting residue was purified by flash column chromatography with 0-5% EtOAc in PE to give the titled compound (490 mg, 72%). ¹H NMR (400 MHz, CDCl₃) δ 6.95 (dd, J=18.4, 7.9 Hz, 2H), 6.87 (s, 1H), 2.94 (s, 2H), 1.46 (s, 6H) ppm.

### Step C: 1-(2,2-dimethyl-2,3-dihydrobenzofuran-6-yl)ethan-1-one

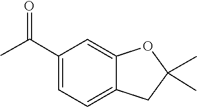

A solution of 6-bromo-2,2-dimethyl-2,3-dihydrobenzofuran (570 mg, 2.51 mmol), ethyl tributylstannanecarboxylate (1.36 g, 3.77 mmol) and Pd(PPh₃)₂Cl₂ (88 mg, 0.13 mmol) in toluene (10 ml) under N₂ was stirred at 100° C. overnight. The solution was cooled to RT. HCl/1,4-dioxane (4M, 2 ml) was added and stirred at RT for 10 min. The solution was diluted with EtOAc (15 ml), washed with aq. Na₂CO₃ (10 ml×2), dried over Na₂SO₄ and concentrated to dryness. The resulting residue was purified by flash column chromatography with 0-10% EtOAc in PE to give the titled compound (0.2 g, 42%). ¹H NMR (400 MHz, CDCl₃) δ 7.46 (d, J=7.8 Hz, 1H), 7.29 (s, 1H), 7.20 (d, J=7.6 Hz, 1H), 3.04 (s, 2H), 2.56 (s, 3H), 1.49 (s, 6H) ppm.

### Step D: 1-(2,2-dimethyl-2,3-dihydrobenzofuran-6-yl)ethan-1-ol

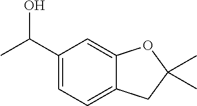

A solution of 1-(2,2-dimethyl-2,3-dihydrobenzofuran-6-yl)ethan-1-one (200 mg, 1.05 mmol) and NaBH₄ (40 mg, 1.05 mmol) in MeOH (3 ml) was stirred at RT for 10 min. The solution was diluted with EtOAc (10 ml), washed with brine (5 ml×2), dried over Na₂SO₄ and concentrated to dryness to give the titled compound (200 mg, 99%), which was used directly for the next step without further purification. MS: M/e 193 (M+1)⁺.

### Step E: 2-(6-((2S,5R)-4-(1-(2,2-dimethyl-2,3-dihydrobenzofuran-6-yl)ethyl)-2,5-dimethylpiperazin-1-yl)-9-ethyl-3-methyl-2-oxo-3,9-dihydro-2H-purin-8-yl)acetonitrile

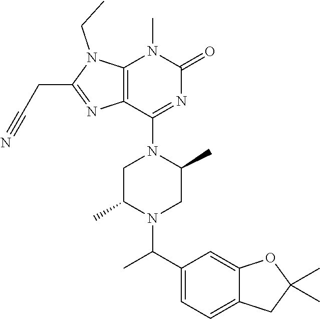 [SMILES: CCn1c(CC#N)nc2c(N3C[C@@H](C)N(C(C)c4ccc5c(c4)OC(C)(C)C5)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (115 mg, 0.35 mmol), 1-(2,2-dimethyl-2,3-dihydrobenzofuran-6-yl)ethan-1-ol (200 mg, 1.04 mmol), (cyanomethyl)trimethylphosphonium iodide (255 mg, 1.05 mmol) and DIPEA (451 mg, 3.50 mmol) in CH₃CN (2 ml) was stirred at 100° C. overnight. The reaction was diluted with EtOAc (15 ml) and washed with brine (10 ml). The organic layer was concentrated under reduced pressure. The resulting residue was purified by flash column chromatography and then Prep-HPLC (Method A) to give the titled compound (73 mg). ¹H NMR (400 MHz, DMSO-d6) δ 7.10 (t, J=8.1 Hz, 1H), 6.80 (d, J=7.1 Hz, 1H), 6.70 (d, J=5.1 Hz, 1H), 6.10-5.40 (m, 1H), 5.20-4.47 (m, 1H), 4.41 (s, 2H), 4.25 (s, 2H), 3.61 (s, 3.5H), 3.52-3.20 (m, 2.5H), 2.97 (d, J=5.5 Hz, 2H), 2.79 (d, J=32.7 Hz, 1.5H), 2.19 (d, J=10.3 Hz, 0.5H), 1.46-1.29 (m, 10.5H), 1.26-1.14 (m, 4.5H), 0.94 (s, 1.5H), 0.80 (d, J=5.3 Hz, 1.5H) ppm. MS: M/e 504 (M+1)⁺.

### Compound A98: 2-(6-((2S,5R)-4-(1-(2,2-dimethylbenzo[d][1,3]dioxol-5-yl)ethyl)-2,5-dimethyl piperazin-1-yl)-3,9-dimethyl-2-oxo-3,9-dihydro-2H-purin-8-yl)acetonitrile

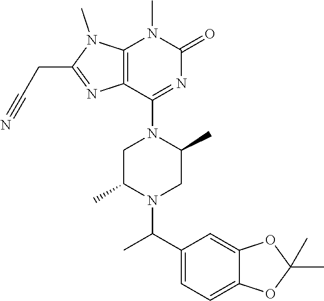 [SMILES: CC(c1ccc2c(c1)OC(C)(C)O2)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (100 mg, 0.32 mmol), 1-(2,2-dimethylbenzo[d][1,3]dioxol-5-yl)ethan-1-ol (62 mg, 0.31 mmol), (cyanomethyl)trimethylphosphonium iodide (230 mg, 0.95 mmol) and DIPEA (205 mg, 1.59 mmol) in MeCN (5 ml) was stirred at 100° C. overnight. After completed, the solution was concentrated under reduced pressure to dryness. The resulting residue was purified by Prep-HPLC (Method A) to give the Compound A98a (34 mg) and Compound A98b (32 mg).

Compound A98: ¹H NMR (400 MHz, CD₃OD) δ 6.83 (s, 1H), 6.77 (t, J=7.0 Hz, 1H), 6.66 (t, J=8.4 Hz, 1H), 6.05-5.02 (m, 2H), 4.21 (s, 1H), 3.93 (s, 3H), 3.78 (s, 3H), 3.75-3.34 (m, 3H), 3.26-2.70 (m, 2H), 2.70-1.92 (m, 1H), 1.64 (d, J=5.1 Hz, 6H), 1.45-1.25 (m, 6H), 1.11-0.88 (m, 3H) ppm. MS: M/e 492 (M+1)⁺

Compound A98a (the earlier peak): ¹H NMR (400 MHz, CD₃OD) δ 6.81 (s, 1H), 6.75 (d, J=7.1 Hz, 1H), 6.65 (d, J=6.9 Hz, 1H), 6.08-5.11 (m, 2H), 3.93 (s, 3H), 3.78 (s, 3H), 3.48 (s, 1H), 3.28-2.66 (m, 6H), 1.64 (d, J=2.2 Hz, 6H), 1.44 (s, 3H), 1.28 (s, 3H), 0.88 (s, 3H) ppm. MS: M/e 492 (M+1)⁺

Compound A98b (the later peak): ¹H NMR (400 MHz, CD₃OD) δ 6.81 (s, 1H), 6.74 (d, J=8.1 Hz, 1H), 6.62 (d, J=7.7 Hz, 1H), 5.47 (d, J=324.8 Hz, 2H), 3.93 (s, 3H), 3.78 (s, 3H), 3.55 (s, 1H), 3.37 (s, 4H), 2.61 (s, 1H), 2.27 (d, J=12.2 Hz, 1H), 1.63 (s, 6H), 1.30 (d, J=5.5 Hz, 3H), 1.27 (s, 3H), 1.01 (d, J=6.2 Hz, 3H) ppm. MS: M/e 492 (M+1)⁺

### Compound A99: 2-(6-((2S,5R)-4-(1-(2,2-difluorobenzo[d][1,3]dioxol-5-yl)ethyl)-2,5-dimethyl piperazin-1-yl)-9-ethyl-3-methyl-2-oxo-3,9-dihydro-2H-purin-8-yl)acetonitrile

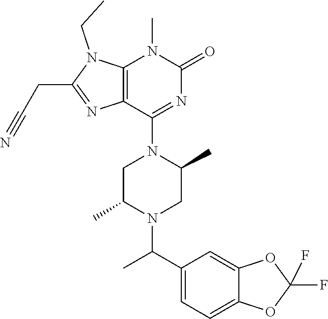 [SMILES: CCn1c(CC#N)nc2c(N3C[C@@H](C)N(C(C)c4ccc5c(c4)OC(F)(F)O5)C[C@@H]3C)nc(=O)n(C)c21]

### Step A: 1-(2,2-difluorobenzo[d][1,3]dioxol-5-yl)ethan-1-ol

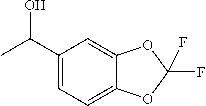

To a solution of 2,2-difluorobenzo[d][1,3]dioxole-5-carbaldehyde (200 mg, 1.07 mmol) in THE (20 mL) was added methylmagnesium bromide (1.3 mL, 1.29 mmol) at −78° C. and stirred for 1 h. The mixture was extracted with DCM (100 mL) and washed with water. The organic layer was dried, concentrated and purified by Prep-TLC (DCM:MeOH=20:1) to give the titled compound (180 mg, 83%). MS: M/e 203 (M+1)⁺

### Step B: 2-(6-((2S,5R)-4-(1-(2,2-difluorobenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile

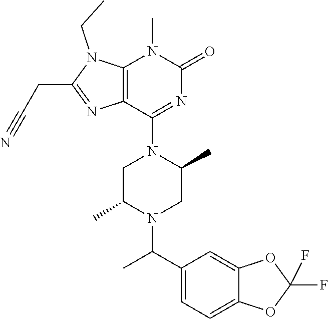 [SMILES: CCn1c(CC#N)nc2c(N3C[C@@H](C)N(C(C)c4ccc5c(c4)OC(F)(F)O5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol) in CH₃CN (3 mL) and was added 1-(2,2-difluorobenzo[d][1,3]dioxol-5-yl)ethan-1-ol (33 mg, 0.15 mmol), (cyanomethyl)trimethylphosphonium iodide (110 mg, 0.46 mmol) and DIPEA (98 mg, 0.76 mmol). The resulting mixture was stirred at 105° C. overnight. The reaction solvent was removed under reduced pressure to give the titled Compound A99 (crude), which was further purified and separated into Compound A99a (10 mg) and Compound A99b (14 mg) by Prep-HPLC (Method A).

Compound A99: ¹H NMR (400 MHz, CD₃OD) δ 7.30 (s, 1H), 7.23-7.08 (m, 2H), 6.35-5.51 (m, 1H), 5.47-4.88 (m, 1H), 4.37 (q, J=7.2 Hz, 2H), 4.23 (s, 2H), 3.76 (s, 3H), 3.73-3.52 (m, 2H), 3.43-3.31 (m, 0.5H), 2.96-2.66 (m, 2H), 2.40-2.20 (m, 0.5H), 1.48-1.43 (m, 4H), 1.40-1.21 (m, 5H), 1.08-0.91 (m, 3H) ppm. MS: M/e 514 (M+1)⁺

Compound A99a (the earlier peak): ¹H NMR (400 MHz, CD₃OD) δ 7.34-7.24 (m, 1H), 7.16 (dd, J=18.2, 8.2 Hz, 2H), 5.88-5.16 (m, 1H), 4.86-4.50 (m, 1H), 4.37 (d, J=6.3 Hz, 2H), 4.22 (s, 1H), 3.76 (s, 3H), 3.67 (s, 1H), 3.62-3.32 (m, 2H), 2.86 (s, 3H), 1.46 (s, 6H), 1.30 (d, J=5.7 Hz, 3H), 0.91 (d, J=5.4 Hz, 3H) ppm. MS: M/e 514 (M+1)⁺

Compound A99b (the later peak): ¹H NMR (400 MHz, CD₃OD) δ 7.29 (s, 1H), 7.16 (d, J=8.0 Hz, 1H), 7.11 (d, J=8.4 Hz, 1H), 5.93-5.04 (m, 1H), 4.86-4.59 (m, 1H), 4.37 (d, J=7.2 Hz, 2H), 4.23 (s, 1H), 3.76 (s, 3H), 3.59 (s, 1H), 3.52 (s, 1H), 3.29-3.07 (m, 2H), 2.66 (s, 1H), 2.19 (d, J=12.1 Hz, 1H), 1.46 (t, J=7.1 Hz, 3H), 1.33 (d, J=5.7 Hz, 3H), 1.27 (s, 3H), 1.04 (d, J=5.1 Hz, 3H) ppm. MS: M/e 514 (M+1)⁺

### Compound A102: 2-(6-((2S,5R)-4-(1-(4,4-difluoro-3,4-dihydro-2H-pyrano[3,2-b]pyridin-6-yl)ethyl)-2,5-dimethylpiperazin-1-yl)-9-ethyl-3-methyl-2-oxo-3,9-dihydro-2H-purin-8-yl)acetonitrile

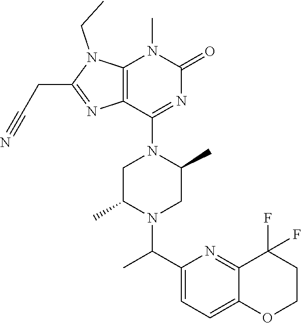 [SMILES: CCn1c(CC#N)nc2c(N3C[C@@H](C)N(C(C)c4ccc5c(n4)C(F)(F)CCO5)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol), 1-(4,4-difluoro-3,4-dihydro-2H-pyrano[3,2-b]pyridin-6-yl)ethan-1-ol (33 mg, 0.15 mmol), (cyanomethyl)trimethylphosphonium iodide (110 mg, 0.46 mmol) and DIPEA (98 mg, 0.76 mmol) in MeCN (3 ml) was stirred at 100° C. overnight. After completed, the solution was concentrated under reduced pressure. The resulting residue was purified by Prep-HPLC (Method A) to give the titled compound (24 mg, 30%). ¹H NMR (400 MHz, CD₃OD) δ 7.67 (t, J=7.6 Hz, 1H), 7.43 (d, J=8.5 Hz, 1H), 6.36-4.80 (m, 2H), 4.47-4.30 (m, 4H), 4.30-3.98 (m, 2H), 3.98-3.80 (m, 1H), 3.77 (s, 3H), 3.74-3.48 (m, 1H), 3.44-3.31 (m, 1H), 3.06-2.84 (m, 2H), 2.63 (d, J=16.0 Hz, 2H), 1.57-1.39 (m, 6H), 1.38-1.28 (m, 3H), 1.19-0.96 (m, 3H) ppm. MS: M/e 527 (M+1)⁺

### Compound A103: 2-(6-((2S,5R)-4-(1-(4,4-difluoro-3,4-dihydro-2H-pyrano[3,2-b]pyridin-8-yl)ethyl)-2,5-dimethylpiperazin-1-yl)-9-ethyl-3-methyl-2-oxo-3,9-dihydro-2H-purin-8-yl)acetonitrile

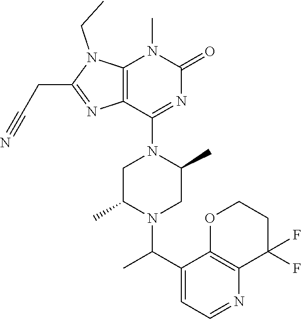 [SMILES: CCn1c(CC#N)nc2c(N3C[C@@H](C)N(C(C)c4ccnc5c4OCCC5(F)F)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol), 1-(4,4-difluoro-3,4-dihydro-2H-pyrano[3,2-b]pyridin-8-yl)ethan-1-ol (33 mg, 0.15 mmol), (cyanomethyl)trimethylphosphonium iodide (110 mg, 0.46 mmol) and DIPEA (98 mg, 0.76 mmol) in MeCN (3 ml) was stirred at 100° C. overnight. After completed, the solution was concentrated under reduced pressure. The resulting residue was purified and separated into Compound A103a (16 mg) and Compound A103b (20 mg) by Prep-HPLC (Method A).

Compound A103a (the earlier peak): ¹H NMR (400 MHz, CD₃OD) δ 8.24 (d, J=4.7 Hz, 1H), 7.80 (d, J=4.7 Hz, 1H), 6.00-4.50 (m, 2H), 4.49-4.40 (m, 2H), 4.37 (q, J=7.2 Hz, 2H), 4.24-3.97 (m, 2H), 3.76 (s, 3H), 3.57 (s, 1H), 3.30-2.36 (m, 5H), 2.16 (d, J=12.1 Hz, 1H), 1.46 (t, J=7.2 Hz, 3H), 1.42-1.20 (m, 6H), 1.05 (d, J=6.0 Hz, 3H) ppm. MS: M/e 527 (M+1)⁺

Compound A103b (the later peak): ¹H NMR (400 MHz, CD₃OD) δ 8.26 (d, J=4.8 Hz, 1H), 7.73 (d, J=4.5 Hz, 1H), 6.23-4.50 (m, 2H), 4.45 (s, 2H), 4.37 (d, J=6.8 Hz, 2H), 4.24-4.17 (m, 2H), 3.76 (s, 3H), 3.30-2.74 (m, 5H), 2.69-2.58 (m, 2H), 1.46 (t, J=7.0 Hz, 6H), 1.27 (d, J=6.6 Hz, 3H), 0.94 (d, J=6.2 Hz, 3H) ppm. MS: M/e 527 (M+1)⁺

### Compound A108: 2-(9-ethyl-6-((2S,5R)-4-(1-(3-isopropylphenyl)ethyl)-2,5-dimethylpiperazin-1-yl)-3-methyl-2-oxo-3,9-dihydro-2H-purin-8-yl)acetonitrile

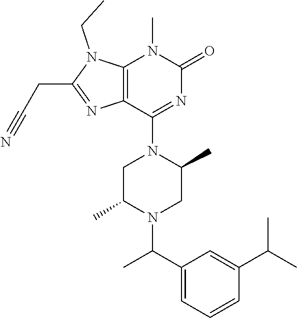 [SMILES: CCn1c(CC#N)nc2c(N3C[C@@H](C)N(C(C)c4cccc(C(C)C)c4)C[C@@H]3C)nc(=O)n(C)c21]

### Step A: 1-(3-isopropylphenyl)ethan-1-ol

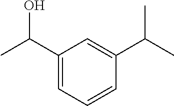

To a mixture of 1-bromo-3-isopropylbenzene (0.6 g, 3 mmol) in THE (10 mL) at −70° C. was added n-BuLi (2 mL, 1.5 mol/L in toluene). After 1 hour, a solution of acetaldehyde (264 mg, 6 mmol) in THE (2 mL) was added. The reaction was stirred for 1 hour. The reaction was quenched with saturated NH₄Cl solution, extracted with EtOAc (60 mL×2), washed with brine, dried over Na₂SO₄, filtered and concentrated. The resulting residue was purified by flash column chromatography to give the titled compound (225 mg, 45%). ¹H NMR (400 MHz, DMSO-d6) δ 7.25-7.18 (m, 2H), 7.13 (d, J=7.2 Hz, 1H), 7.08 (d, J=7.6 Hz, 1H), 5.08 (brs, 1H), 4.74-4.62 (m, 1H), 2.91-2.80 (m, 1H), 1.30 (d, J=6.4 Hz, 3H), 1.19 (d, J=7.2 Hz, 6H) ppm.

### Step B: 2-(9-ethyl-6-((2S,5R)-4-(1-(3-isopropylphenyl)ethyl)-2,5-dimethylpiperazin-1-yl)-3-methyl-2-oxo-3,9-dihydro-2H-purin-8-yl)acetonitrile

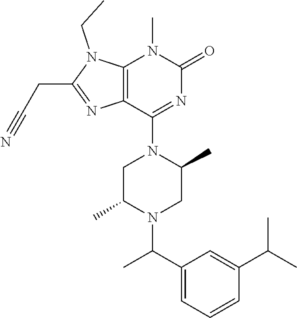 [SMILES: CCn1c(CC#N)nc2c(N3C[C@@H](C)N(C(C)c4cccc(C(C)C)c4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol), 1-(3-isopropylphenyl)ethan-1-ol (75 mg, 0.45 mmol) and (cyanomethyl) trimethyl phosphonium iodide (218 mg, 0.9 mmol) in CH₃CN (4 mL) was added DIPEA (387 mg, 3 mmol). The mixture was sealed in a bottle and heated at 100° C. for 16 hours. The mixture was cooled to room temperature, diluted with water, extracted with EtOAc (60 mL×2), washed with brine, dried over Na₂SO₄, filtered, and concentrated to dryness. The resulting residue was purified by Prep-HPLC (Method A) to give the titled Compound A108 (60 mg, 41%), which was further separated into Compound A108a (12 mg) and Compound A108b (15 mg) by chiral-Prep-HPLC. The chiral separation conditions are shown below.

| | |
|---|---|
| Column | CHIRALPAK IH |
| Column Size | 2 cm × 25 cm, 5 um |
| Mobile Phase A | Hex:DCM |
| Mobile Phase B | EtOH |
| Flow Rate | 20 mL/min |
| Wave Length | UV 220 nm |
| Temperature | 25° C. |
| Prep-HPLC Equipment | Prep-HPLC-Gilson |

Compound A108: ¹H NMR (400 MHz, CD₃OD) δ 7.40-7.06 (m, 4H), 6.39-5.45 (m, 1H), 5.25-4.95 (m, 1H), 4.44-4.31 (m, 2H), 4.28-4.16 (m, 1.5H), 3.85-3.73 (m, 3.5H), 3.72-3.55 (m, 1.5H), 3.35-3.31 (m, 1H), 3.10-2.84 (m, 2.5H), 2.82-2.68 (m, 0.5H), 2.37-2.27 (m, 0.5H), 1.53-1.35 (m, 7H), 1.32-1.21 (m, 8H), 1.14-1.06 (m, 1.5H), 1.02-0.90 (m, 1.5H) ppm. MS: M/e 476 (M+1)⁺.

Compound A108a (the earlier peak): ¹H NMR (400 MHz, CD₃OD) δ 7.27-7.06 (m, 4H), 6.39-5.41 (m, 1H), 5.25-4.90 (m, 1H), 4.42-4.30 (m, 2H), 3.76 (s, 3H), 3.65-3.50 (m, 1H), 3.34-3.25 (m 3H), 2.95-2.76 (m, 4H), 1.54-1.38 (m, 6H), 1.36-1.21 (m, 9H), 0.87 (d, J=6.0 Hz, 3H) ppm. MS: M/e 476 (M+1)⁺.

Compound A108b (the later peak): ¹H NMR (400 MHz, CD₃OD) δ 7.30 (s, 1H), 7.20 (t, J=7.2 Hz, 1H), 7.13 (d, J=6.8 Hz, 1H), 7.09 (d, J=6.8 Hz, 1H), 6.15-5.65 (m, 1H), 5.27-4.95 (m, 1H), 4.45-4.30 (m, 2H), 3.76 (s, 3H), 3.66-3.54 (m, 1H), 3.50-3.39 (m, 1H), 3.34-3.25 (m 3H), 2.96-2.82 (m, 1H), 2.71-2.52 (m, 1H), 2.27-2.16 (m, 1H), 1.46 (t, J=7.2 Hz, 3H), 1.38-1.19 (m, 12H), 1.03 (d, J=5.6 Hz, 3H) ppm. MS: M/e 476 (M+1)⁺.

### Compound A110: 2-(6-((2S,5R)-4-(1-(3-(tert-butyl)phenyl)ethyl)-2,5-dimethylpiperazin-1-yl)-9-ethyl-3-methyl-2-oxo-3,9-dihydro-2H-purin-8-yl)acetonitrile

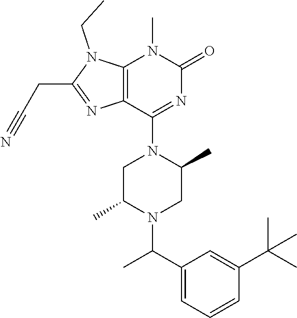 [SMILES: CCn1c(CC#N)nc2c(N3C[C@@H](C)N(C(C)c4cccc(C(C)(C)C)c4)C[C@@H]3C)nc(=O)n(C)c21]

### Step A: 1-(3-(tert-butyl)phenyl)ethan-1-ol

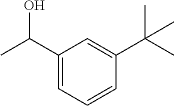

To a mixture of 1-bromo-3-(tert-butyl)benzene (0.64 g, 3 mmol) in THE (10 mL) at −70° C. was added n-BuLi (2 mL, 1.5 mol/L in toluene). After 1 hour, a solution of acetaldehyde (264 mg, 6 mmol) in THE (2 mL) was added. The reaction was stirred for 1 hour. The reaction was quenched with saturated NH₄Cl, extracted with EtOAc (60 mL×2), washed with brine, dried over Na₂SO₄, filtered and concentrated. The resulting residue was purified by flash column chromatography to give the titled compound (100 mg, 19%). ¹H NMR (400 MHz, DMSO-d6) δ 7.36 (s, 1H), 7.26-7.20 (m, 2H), 7.16-7.10 (m, 1H), 5.08 (d, J=4.0 Hz, 1H), 4.74-4.62 (m, 1H), 1.31 (d, J=6.4 Hz, 3H), 1.27 (s, 9H) ppm.

### Step B: 2-(6-((2S,5R)-4-(1-(3-(tert-butyl)phenyl)ethyl)-2,5-dimethylpiperazin-1-yl)-9-ethyl-3-methyl-2-oxo-3,9-dihydro-2H-purin-8-yl)acetonitrile

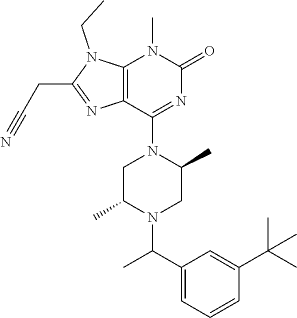 [SMILES: CCn1c(CC#N)nc2c(N3C[C@@H](C)N(C(C)c4cccc(C(C)(C)C)c4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol), 1-(3-(tert-butyl)phenyl)ethan-1-ol (100 mg, 0.56 mmol) and (cyanomethyl) trimethyl phosphonium iodide (218 mg, 0.9 mmol) in CH₃CN (4 mL) was added DIPEA (387 mg, 3 mmol). The mixture was sealed in a bottle and heated at 100° C. for 16 hours. The mixture was cooled to room temperature, diluted with water, extracted with EtOAc (60 mL×2), washed with brine, dried over Na₂SO₄, filtered, and concentrated to dryness. The resulting residue was purified by Prep-HPLC (Method A) to give the titled compound (65 mg, 44%). ¹H NMR (400 MHz, CD₃OD) δ 7.49 (d, J=8.0 Hz, 1H), 7.36-7.19 (m, 2H), 7.15 (d, J=7.6 Hz, 1H), 6.20-5.60 (m, 1H), 5.25-4.95 (m, 1H), 4.37 (q, J=7.2 Hz 2H), 4.28-4.16 (m, 1.5H), 3.86-3.73 (m, 3.5H), 3.71-3.55 (m, 1.5H), 3.35-3.30 (m, 1H), 3.10-2.89 (m, 1.5H), 2.80-2.66 (m, 0.5H), 2.34-2.24 (m, 0.5H), 1.53-1.36 (m, 7.5H), 1.33 (s, 4.5H), 1.32 (s, 4.5H), 1.31-1.24 (m, 1.5H), 1.12-1.06 (m, 1.5H), 1.02-0.90 (m, 1.5H) ppm. MS: M/e 490 (M+1)⁺.

### Compound A112: 2-(6-((2S,5R)-4-(1-(3-(difluoromethyl)-4-fluorophenyl)ethyl)-2,5-dimethylpiperazin-1-yl)-9-ethyl-3-methyl-2-oxo-3,9-dihydro-2H-purin-8-yl)acetonitrile

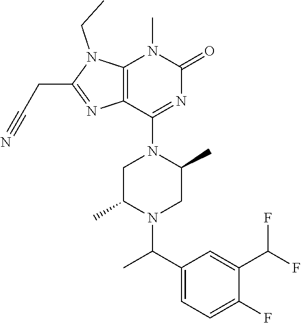 [SMILES: CCn1c(CC#N)nc2c(N3C[C@@H](C)N(C(C)c4ccc(F)c(C(F)F)c4)C[C@@H]3C)nc(=O)n(C)c21]

### Step A: 4-bromo-2-(difluoromethyl)-1-fluorobenzene

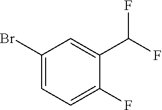 [SMILES: Fc1ccc(Br)cc1C(F)F]

A mixture of 5-bromo-2-fluorobenzaldehyde (1 g, 5 mmol), DAST (1.6 g, 10 mmol) in DCM (15 mL) was stirred at room temperature for 5 hours. The solution was diluted with DCM and washed with water. The organic layer was dried and purified by flash column chromatography to give the titled compound (1 g, 90%). MS: M/e 225 (M+1)⁺

### Step B: 1-(3-(difluoromethyl)-4-fluorophenyl)ethan-1-one

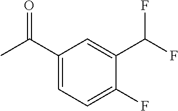

A mixture of 4-bromo-2-(difluoromethyl)-1-fluorobenzene (1 g, 4.5 mmol), tributyl(1-ethoxyvinyl)stannane (2.4 g, 6.7 mmol) and Pd(PPh₃)₂Cl₂ (312 mg, 0.45 mmol) in toluene (15 mL) was stirred at 100° C. under N₂ for 16 hours. The reaction mixture was diluted with HCl (4 mL, 3 M in ethyl acetate) in drops and the mixture was stirred at room temperature for 1 hour. The reaction was concentrated to dryness. The resulting residue was purified by flash column chromatography to give the titled compound (600 mg, 71%). MS: M/e 189 (M+1)⁺

### Step C: 1-(3-(difluoromethyl)-4-fluorophenyl)ethan-1-ol

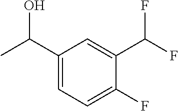

To a solution of 1-(3-(difluoromethyl)-4-fluorophenyl)ethan-1-one (600 mg, 3.1 mmol) in MeOH (10 mL) was added NaBH₄ (120 mg, 3.1 mmol) at room temperature and the resulting mixture was stirred at room temperature for 5 min. The reaction mixture was diluted with DCM and washed with water, dried over Na₂SO₄ and concentrated to give the titled compound (330 mg). MS: M/e 191 (M+1)⁺.

### Step D: 2-(6-((2S,5R)-4-(1-(3-(difluoromethyl)-4-fluorophenyl)ethyl)-2,5-dimethylpiperazin-1-yl)-9-ethyl-3-methyl-2-oxo-3,9-dihydro-2H-purin-8-yl)acetonitrile

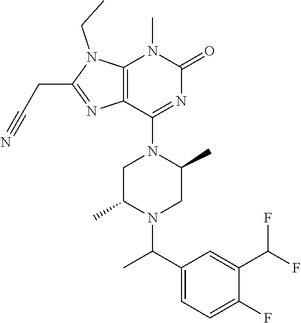 [SMILES: CCn1c(CC#N)nc2c(N3C[C@@H](C)N(C(C)c4ccc(F)c(C(F)F)c4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol) in CH₃CN (3 mL) and was added 1-(3-(difluoromethyl)-4-fluorophenyl)ethan-1-ol (116 mg, 0.6 mmol), (cyanomethyl)trimethylphosphonium iodide (219 mg, 0.9 mmol) and DIPEA (387 mg, 3 mmol). The resulting mixture was stirred at 105° C. overnight. The reaction solvent was removed under reduced pressure. The resulting residue was purified by flash column chromatography to give the titled compound (crude), which was further purified to give the titled compound (33 mg) by Prep-HPLC (Method A). ¹H NMR (400 MHz, CD₃OD) δ 7.73-7.52 (m, 2H), 7.25-7.14 (m, 1H), 7.13-6.82 (m, 1H), 6.28-4.98 (m, 2H), 4.37 (d, J=7.1 Hz, 2H), 4.23 (s, 2H), 3.76 (s, 3H), 3.74-3.36 (m, 2H), 3.11-2.75 (m, 2H), 2.75-2.06 (m, 1H), 1.49-1.42 (m, 4H), 1.39-1.21 (m, 5H), 1.11-0.85 (m, 3H) ppm. MS: M/e 502 (M+1)⁺.

### Compound A113: 2-(9-ethyl-6-((2S,5R)-4-(1-(4-fluoro-3-(trifluoromethyl)phenyl)ethyl)-2,5-dimethylpiperazin-1-yl)-3-methyl-2-oxo-3,9-dihydro-2H-purin-8-yl)acetonitrile

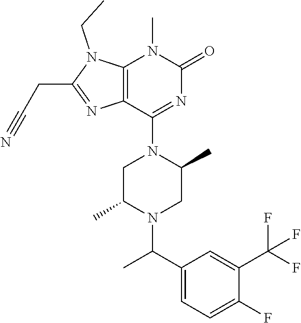 [SMILES: CCn1c(CC#N)nc2c(N3C[C@@H](C)N(C(C)c4ccc(F)c(C(F)(F)F)c4)C[C@@H]3C)nc(=O)n(C)c21]

### Step A: 1-(4-fluoro-3-(trifluoromethyl)phenyl)ethan-1-ol

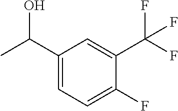

To a solution of 1-(4-fluoro-3-(trifluoromethyl)phenyl)ethan-1-one (100 mg, 0.48 mmol) in MeOH (2 ml) at 0° C. was added NaBH₄ (18 mg, 0.48 mmol) in some portions. After addition, the reaction was stirred at 0° C. for 5 min. The reaction was quenched by adding water and extracted with DCM, washed with brine, dried over Na₂SO₄, filtered, concentrated to give the titled compound (100 mg, crude). MS: M/e 191 (M+1-18)⁺.

### Step B: 2-(9-ethyl-6-((2S,5R)-4-(1-(4-fluoro-3-(trifluoromethyl)phenyl)ethyl)-2,5-dimethyl piperazin-1-yl)-3-methyl-2-oxo-3,9-dihydro-2H-purin-8-yl)acetonitrile

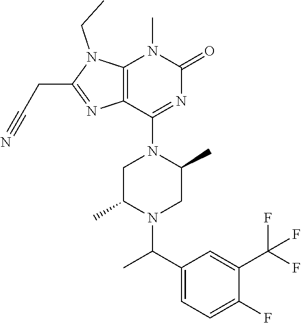 [SMILES: CCn1c(CC#N)nc2c(N3C[C@@H](C)N(C(C)c4ccc(F)c(C(F)(F)F)c4)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mol), 1-(4-fluoro-3-(trifluoromethyl)phenyl)ethan-1-ol (94 mg, 0.45 mol), (cyanomethyl)trimethylphosphonium iodide (218 mg, 0.9 mol) and DIPEA (387 mg, 3 mol) in CH₃CN (2 ml). Then the mixture solution was stirred at 105° C. for 16 hours. The reaction mixture was concentrated under reduced pressure and purified by flash column chromatography (DCM:MeOH) to give the titled compound (39.1 mg). ¹H NMR (400 MHz, CD₃OD) δ 7.80-7.61 (m, 2H), 7.35-7.22 (m, 1H), 6.50-4.99 (m, 2H), 4.37 (q, J=7.0 Hz, 2H), 4.22 (s, 1H), 3.76 (s, 3H), 3.74-3.65 (m, 1H), 3.60-3.48 (m, 1H), 3.30-3.21 (m, 1H), 2.99-2.73 (m, 2H), 2.70-2.03 (m, 1H), 1.46 (t, J=7.0 Hz, 4H), 1.37-1.21 (m, 5H), 1.25-0.78 (m, 3H) ppm. MS: M/e 520 (M+1)⁺.

### Compound A115: 2-(6-((2S,5R)-2,5-dimethyl-4-(1-(4-methyl-3-(trifluoromethyl)phenyl)ethyl) piperazin-1-yl)-9-ethyl-3-methyl-2-oxo-3,9-dihydro-2H-purin-8-yl)acetonitrile

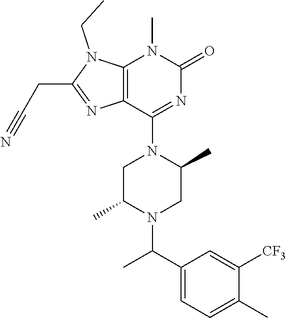 [SMILES: CCn1c(CC#N)nc2c(N3C[C@@H](C)N(C(C)c4ccc(C)c(C(F)(F)F)c4)C[C@@H]3C)nc(=O)n(C)c21]

### Step A: 1-(4-methyl-3-(trifluoromethyl)phenyl)ethan-1-one

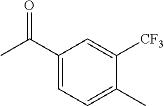

A solution of 4-bromo-1-methyl-2-(trifluoromethyl)benzene (500 mg, 2.09 mmol), Pd(PPh₃)₂Cl₂ (73 mg, 0.10 mmol) and tributyl(1-ethoxyvinyl)stannane (1.13 g, 3.13 mmol) in Tol. (10 ml) was stirred at 100° C. overnight. The mixture was cooled to RT and HCl (4M in dioxane, 2 ml) was added, stirred for 30 min. The mixture was diluted with EtOAc (20 ml), washed with brine (10 ml), dried and concentrated. The residue was purified by flash column chromatography with 0%-5% EtOAc in PE to give the titled compound (200 mg, 47%). ¹H NMR (400 MHz, CDCl₃) δ 8.20 (s, 1H), 8.01 (d, J=7.6 Hz, 1H), 7.40 (d, J=7.8 Hz, 1H), 2.62 (s, 3H), 2.56 (s, 3H) ppm.

### Step B: 1-(4-methyl-3-(trifluoromethyl)phenyl)ethan-1-ol

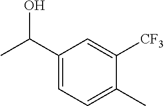

A solution of 1-(4-methyl-3-(trifluoromethyl)phenyl)ethan-1-one (200 mg, 0.99 mmol) and NaBH₄ (38 mg, 1.00 mmol) in MeOH (4 ml) was stirred at RT for 30 min. The solution was diluted with EtOAc (15 ml), washed with brine (10 ml), dried and evaporated to dryness to give the titled compound (200 mg, 100%), which was used directly for the next step. MS: M/e 205 (M+1)⁺.

### Step C: 2-(6-((2S,5R)-2,5-dimethyl-4-(1-(4-methyl-3-(trifluoromethyl)phenyl)ethyl)piperazin-1-yl)-9-ethyl-3-methyl-2-oxo-3,9-dihydro-2H-purin-8-yl)acetonitrile

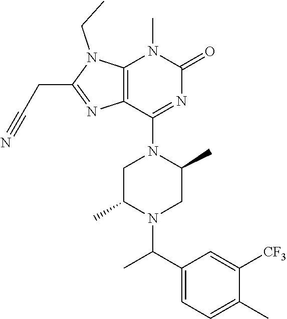 [SMILES: CCn1c(CC#N)nc2c(N3C[C@@H](C)N(C(C)c4ccc(C)c(C(F)(F)F)c4)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (108 mg, 0.33 mmol), 1-(4-methyl-3-(trifluoromethyl)phenyl)ethan-1-ol (200 mg, 0.98 mmol), (cyanomethyl)trimethylphosphonium iodide (239 mg, 0.98 mmol) and DIPEA (423 mg, 3.28 mmol) in MeCN (2 ml) was stirred at 100° C. overnight. The reaction was diluted with EtOAc (10 ml) and washed with brine (10 ml). The organic layer was concentrated under reduced pressure. The resulting residue was purified by flash column chromatography with 0-5% MeOH in DCM and then Prep-HPLC (Method A) to give the titled compound (44 mg). ¹H NMR (400 MHz, DMSO-d6) δ 7.68 (d, J=19.1 Hz, 1H), 7.56 (dd, J=22.6, 8.1 Hz, 1H), 7.44-7.36 (m, 1H), 6.15-5.35 (m, 1H), 5.20-4.55 (m, 1H), 4.41 (s, 2H), 4.26 (d, J=5.8 Hz, 2H), 3.68 (q, J=6.4 Hz, 1H), 3.61 (s, 3H), 3.52 (q, J=5.5 Hz, 1H), 3.32 (s, 1H), 2.75 (s, 1.5H), 2.43 (s, 3H), 2.05 (d, J=12.3 Hz, 0.5H), 1.34 (t, J=6.7 Hz, 4.5H), 1.25 (t, J=6.2 Hz, 3H), 1.16 (s, 1.5H), 0.96 (s, 1.5H), 0.83 (d, J=5.7 Hz, 1.5H) ppm. MS: M/e 516 (M+1)⁺.

### Compound A117: 2-(9-ethyl-6-((2S,5R)-4-(1-(6-isopropoxypyridin-2-yl)ethyl)-2,5-dimethyl piperazin-1-yl)-3-methyl-2-oxo-3,9-dihydro-2H-purin-8-yl)acetonitrile

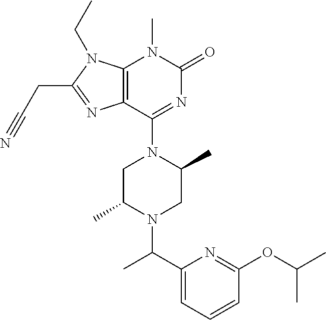 [SMILES: CCn1c(CC#N)nc2c(N3C[C@@H](C)N(C(C)c4cccc(OC(C)C)n4)C[C@@H]3C)nc(=O)n(C)c21]

### Step A: 1-(6-isopropoxyyridin-2-yl)ethan-1-ol

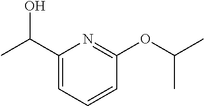

To a solution of 1-(6-isopropoxypyridin-2-yl)ethan-1-one (150 mg, 0.84 mmol) in MeOH (15 mL) was added NaBH₄ (32 mg, 0.84 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (100 mg, 66%). MS: M/e 182 (M+1)⁺.

### Step B: 2-(9-ethyl-6-((2S,5R)-4-(1-(6-isopropoxypyridin-2-yl)ethyl)-2,5-dimethylpiperazin-1-yl)-3-methyl-2-oxo-3,9-dihydro-2H-purin-8-yl)acetonitrile

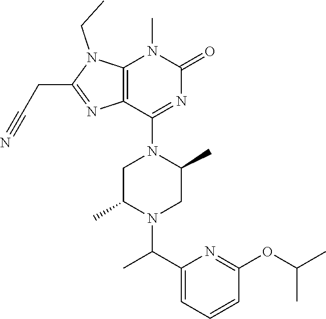 [SMILES: CCn1c(CC#N)nc2c(N3C[C@@H](C)N(C(C)c4cccc(OC(C)C)n4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol) in CH₃CN (3 mL) and was added 1-(6-isopropoxypyridin-2-yl)ethan-1-ol (28 mg, 0.15 mmol), (cyanomethyl)trimethylphosphonium iodide (110 mg, 0.46 mmol) and DIPEA (98 mg, 0.76 mmol). The resulting mixture was stirred at 105° C. overnight. The reaction solvent was removed under reduced pressure to dryness. The resulting residue was purified and separated into Compound A117a (26 mg) and Compound A117b (22 mg) by Prep-HPLC (Method A).

Compound A117a (the earlier peak): ¹H NMR (400 MHz, CD₃OD) δ 7.62 (t, J=7.9 Hz, 1H), 7.07 (d, J=7.4 Hz, 1H), 6.59 (d, J=8.2 Hz, 1H), 5.95 (s, 1H), 5.30-5.22 (m, 1H), 4.86 (s, 2H), 4.85-4.44 (m, 1H), 4.37 (q, J=7.2 Hz, 2H), 3.76 (s, 3H), 3.74-3.36 (m, 3H), 2.87 (s, 1H), 2.38 (d, J=11.8 Hz, 1H), 1.46 (t, J=7.2 Hz, 3H), 1.38 (d, J=6.5 Hz, 3H), 1.35-1.27 (m, 9H), 1.07 (d, J=6.1 Hz, 3H) ppm. MS: M/e 493 (M+1)⁺

Compound A117b (the later peak): ¹H NMR (400 MHz, CD₃OD) δ 7.62 (t, J=7.7 Hz, 1H), 7.02 (d, J=7.3 Hz, 1H), 6.59 (d, J=8.2 Hz, 1H), 5.87-5.32 (m, 1H), 5.30-5.24 (m, 1H), 4.86 (s, 2H), 4.85-4.48 (m, 1H), 4.37 (q, J=7.0 Hz, 2H), 3.79 (s, 1H), 3.76 (s, 3H), 3.43 (s, 1H), 3.03 (s, 2H), 2.83 (d, J=10.9 Hz, 1H), 1.46 (t, J=7.2 Hz, 3H), 1.39 (dd, J=10.1, 6.9 Hz, 6H), 1.33 (d, J=6.0 Hz, 6H), 1.01 (d, J=6.4 Hz, 3H) ppm. MS: M/e 493 (M+1)⁺

### Compound A123: 2-(9-ethyl-6-((2S,5R)-4-(1-(6-isopropylpyridin-2-yl)ethyl)-2,5-dimethylpiperazin-1-yl)-3-methyl-2-oxo-3,9-dihydro-2H-purin-8-yl)acetonitrile

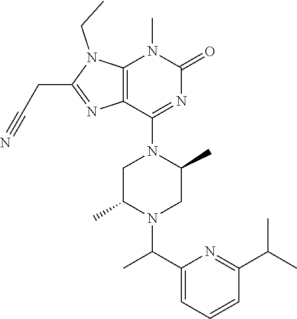 [SMILES: CCn1c(CC#N)nc2c(N3C[C@@H](C)N(C(C)c4cccc(C(C)C)n4)C[C@@H]3C)nc(=O)n(C)c21]

### Step A: 1-(6-isoproyloyridin-2-yl)ethan-1-ol

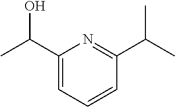

To a solution of 1-(6-isopropylpyridin-2-yl)ethan-1-one (150 mg, 0.92 mmol) in MeOH (15 mL) was added NaBH₄ (70 mg, 1.84 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (120 mg, 79%). MS: M/e 166 (M+1)⁺.

### Step B: 2-(9-ethyl-6-((2S,5R)-4-(1-(6-isopropylpyridin-2-yl)ethyl)-2,5-dimethylpiperazin-1-yl)-3-methyl-2-oxo-3,9-dihydro-2H-purin-8-yl)acetonitrile

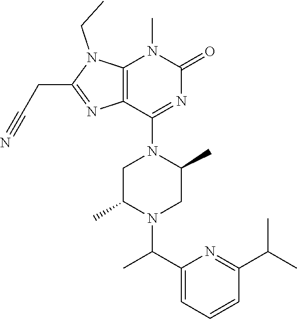 [SMILES: CCn1c(CC#N)nc2c(N3C[C@@H](C)N(C(C)c4cccc(C(C)C)n4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol) in CH₃CN (3 mL) and was added 1-(6-isopropylpyridin-2-yl)ethan-1-ol (25 mg, 0.15 mmol), (cyanomethyl)trimethylphosphonium iodide (110 mg, 0.46 mmol) and DIPEA (98 mg, 0.76 mmol). The resulting mixture was stirred at 105° C. overnight. The reaction solvent was removed under reduced pressure to dryness. The resulting residue was purified by Prep-HPLC (Method A) to give the titled compound (28 mg, 38%). ¹H NMR (400 MHz, CD₃OD) δ 7.77 (d, J=7.5 Hz, 1H), 7.41 (t, J=9.3 Hz, 1H), 7.31-7.20 (m, 1H), 6.45-4.89 (m, 2H), 4.37 (d, J=6.9 Hz, 2H), 4.25-4.02 (m, 2H), 3.83 (s, 1H), 3.77 (s, 3H), 3.62-3.31 (m, 1.5H), 3.16-2.99 (m, 3H), 2.50-2.20 (m, 0.5H), 1.51-1.44 (m, 6H), 1.43-1.28 (m, 9H), 1.25-1.03 (m, 3H) ppm. MS: M/e 477 (M+1)⁺

### Compound A124: 2-(9-ethyl-6-((2S,5R)-4-(1-(2-isopropoxypyridin-4-yl)ethyl)-2,5-dimethylpiperazin-1-yl)-3-methyl-2-oxo-3,9-dihydro-2H-purin-8-yl)acetonitrile

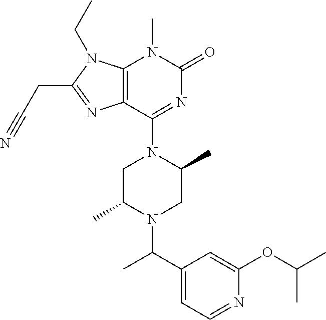 [SMILES: CCn1c(CC#N)nc2c(N3C[C@@H](C)N(C(C)c4ccnc(OC(C)C)c4)C[C@@H]3C)nc(=O)n(C)c21]

### Step A: 1-(2-isopropoxyyridin-4-yl)ethan-1-ol

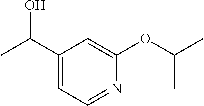

To a solution of 1-(2-isopropoxypyridin-4-yl)ethan-1-one (150 mg, 0.84 mmol) in MeOH (15 mL) was added NaBH₄ (32 mg, 0.84 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (110 mg, 73%). MS: M/e 182 (M+1)⁺.

### Step B: 2-(9-ethyl-6-((2S,5R)-4-(1-(2-isopropoxypyridin-4-yl)ethyl)-2,5-dimethylpiperazin-1-yl)-3-methyl-2-oxo-3,9-dihydro-2H-purin-8-yl)acetonitrile

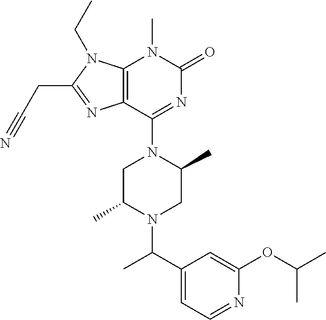 [SMILES: CCn1c(CC#N)nc2c(N3C[C@@H](C)N(C(C)c4ccnc(OC(C)C)c4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol) in CH₃CN (3 mL) and was added 1-(2-isopropoxypyridin-4-yl)ethan-1-ol (28 mg, 0.15 mmol), (cyanomethyl)trimethylphosphonium iodide (110 mg, 0.46 mmol) and DIPEA (98 mg, 0.76 mmol). The resulting mixture was stirred at 105° C. overnight. The reaction solvent was removed under reduced pressure to dryness. The resulting residue was further purified to give the titled compound (44 mg, 59%). ¹H NMR (400 MHz, CD₃OD) δ 8.03 (dd, J=10.4, 5.3 Hz, 1H), 6.99 (d, J=4.8 Hz, 1H), 6.76 (s, 1H), 6.40-5.25 (m, 2H), 5.22-5.14 (m, 1H), 4.41-4.33 (m, 2H), 4.23 (s, 2H), 3.76 (s, 3H), 3.64-3.43 (m, 2H), 3.30-3.27 (m, 0.5H), 2.92-2.67 (m, 2H), 2.30-2.10 (m, 0.5H), 1.49-1.31 (m, 12H), 1.29 (d, J=6.2 Hz, 3H), 1.07-0.89 (m, 3H) ppm. MS: M/e 493 (M+1)⁺

### Compound A125: 2-(6-((2S,5R)-4-(1-(3-cyclopropylphenyl)ethyl)-2,5-dimethylpiperazin-1-yl)-9-ethyl-3-methyl-2-oxo-3,9-dihydro-2H-purin-8-yl)acetonitrile

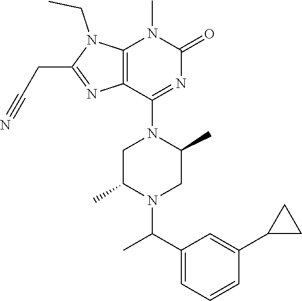 [SMILES: CCn1c(CC#N)nc2c(N3C[C@@H](C)N(C(C)c4cccc(C5CC5)c4)C[C@@H]3C)nc(=O)n(C)c21]

### Step A: 1-(3-cycloproylphenyl)ethan-1-ol

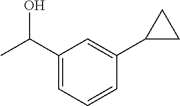

To a stirred solution of 1-bromo-3-cyclopropylbenzene (394 mg, 2 mmol) in THE (8 mL) was added dropwise n-BuLi (1.6 M, 1.25 mL, 2 mmol) at −78° C. After stirred for 30 min, a solution of acetaldehyde (176 mg, 4 mmol) in THE (2 mL) was added dropwise. After the addition, the reaction mixture was stirred for an hour. The reaction was quenched with aq.NH₄Cl, extracted with EtOAc (10 mL×3). The combined organic layers were washed with brine, dried over Na₂SO₄, concentrated and purified by flash column chromatography to give the titled compound (280 mg, 86%). ¹H NMR (400 MHz, DMSO-d6) δ 7.16 (t, J=7.5 Hz, 1H), 7.10-7.03 (m, 2H), 6.89 (d, J=7.6 Hz, 1H), 5.07 (d, J=4.0 Hz, 1H), 4.71-4.60 (m, 1H), 1.94-1.81 (m, 1H), 1.29 (d, J=6.4 Hz, 3H), 0.98-0.89 (m, 2H), 0.69-0.60 (m, 2H) ppm.

### Step B: 2-(6-((2S,5R)-4-(1-(3-cyclopropylphenyl)ethyl)-2,5-dimethylpiperazin-1-yl)-9-ethyl-3-methyl-2-oxo-3,9-dihydro-2H-purin-8-yl)acetonitrile

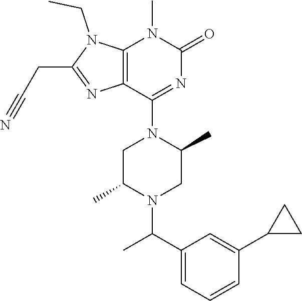 [SMILES: CCn1c(CC#N)nc2c(N3C[C@@H](C)N(C(C)c4cccc(C5CC5)c4)C[C@@H]3C)nc(=O)n(C)c21]

A mixture of 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol), 1-(3-cyclopropylphenyl)ethan-1-ol (84.1 mg, 0.45 mmol), (cyanomethyl)trimethylphosphonium iodide (218 mg, 0.9 mmol) and DIPEA (387 mg, 3 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to give the residue, which was purified by Prep-HPLC (Method A) to give the titled compound (55 mg). ¹H NMR (400 MHz, CD₃OD) δ 7.28-7.12 (m, 3H), 6.99 (d, J=7.2 Hz, 1H), 6.32-4.96 (m, 2H), 4.44-4.32 (m, 2H), 4.23 (s, 1H), 3.86-3.79 (m, 0.5H), 3.76 (s, 3H), 3.73-3.20 (m, 3H), 3.12-2.65 (m, 2H), 2.34 (d, J=12.4 Hz, 0.5H), 1.96-1.85 (m, 1H), 1.50-0.62 (m, 16H) ppm. MS: M/e 474 (M+1)⁺.

### Compound A126: 2-(6-((2S,5R)-2,5-dimethyl-4-(1-(3-(1-methylcyclopropyl)phenyl)ethyl) piperazin-1-yl)-9-ethyl-3-methyl-2-oxo-3,9-dihydro-2H-purin-8-yl)acetonitrile

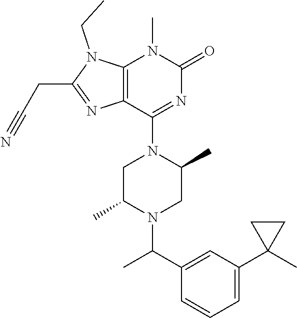 [SMILES: CCn1c(CC#N)nc2c(N3C[C@@H](C)N(C(C)c4cccc(C5(C)CC5)c4)C[C@@H]3C)nc(=O)n(C)c21]

### Step A: 1-(3-(prop-1-en-2-yl)phenyl)ethan-1-one

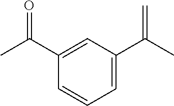

To a solution of 1-(3-bromophenyl)ethan-1-one (2 g, 10 mmol) and potassium isopropenyltrifluoroborate (1.5 g, 10 mmol) in IPA/H₂O (2/1, 20 mL) was added TEA (1.5 g, 15 mmol) and Pd(dppf)₂Cl₂ (292 mg, 0.4 mmol). The reaction mixture was protected by N₂ atmosphere and stirred at 80° C. overnight. The mixture was cooled down to RT, diluted with water, extracted with EtOAc (80 mL×2), washed with brine, dried over Na₂SO₄, filtered, and concentrated to dryness. The residue was purified by flash column chromatography to give the titled compound (1.3 g, 81%). ¹H NMR (400 MHz, CDCl₃) δ 8.05 (s, 1H), 7.86 (d, J=7.2 Hz, 1H), 7.67 (d, J=7.2 Hz, 1H), 7.43 (t, J=7.6 Hz, 1H), 5.43 (s, 1H), 5.17 (s, 1H), 2.63 (s, 3H), 2.19 (s, 3H) ppm.

### Step B: 1-(3-(1-methylcyclopropyl)phenyl)ethan-1-one

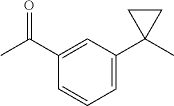

To a solution of Et₂Zn (4 mL, 1 mol/L in hexane) in DCM (4 mL) was added a solution of TFA (456 mg, 4 mmol) in DCM (4 mL) drop wise at ice-bath. After 20 min, a solution of CH₂I₂ (1.07 g, 4 mmol) in DCM (4 mL) was added drop wise. Then 20 min later, a solution of 1-(3-(prop-1-en-2-yl)phenyl)ethan-1-one (320 mg, 2 mmol) in DCM (4 mL) was added drop wise. Then the reaction mixture was warmed to room temperature overnight. The reaction mixture was diluted with H₂O, extracted with DCM (60 mL), washed with brine, dried over Na₂SO₄, filtered, and concentrated to dryness. The residue was purified by flash column chromatography to give the titled compound (130 mg, 37%). ¹H NMR (400 MHz, CDCl₃) δ 7.86 (s, 1H), 7.75 (d, J=7.6 Hz, 1H), 7.46 (d, J=7.2 Hz, 1H), 7.37 (t, J=7.2 Hz, 1H), 2.60 (s, 3H), 1.43 (s, 3H), 0.94-0.86 (m, 2H), 0.81-0.74 (m, 2H) ppm.

### Step C: 1-(3-(1-methylcyclopropyl)phenyl)ethan-1-ol

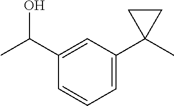

To a solution of 1-(3-(1-methylcyclopropyl)phenyl)ethan-1-one (130 mg, 0.75 mmol) in MeOH (5 mL) was added NaBH₄ (28 mg, 0.75 mmol) slowly at 0° C. The resulting mixture was stirred at room temperature for 0.5 hour. The reaction mixture was quenched with H₂O (10 mL) and extracted with EtOAc (80 mL), washed with brine, dried over Na₂SO₄, filtered, and concentrated to dryness. The residue was purified by flash column chromatography to give the product (125 mg, 96% yield). MS: M/e 177 (M+1)⁺.

### Step D: 2-(6-((2S,5R)-2,5-dimethyl-4-(1-(3-(1-methylcyclopropyl)phenyl)ethyl)piperazin-1-yl)-9-ethyl-3-methyl-2-oxo-3,9-dihydro-2H-purin-8-yl)acetonitrile

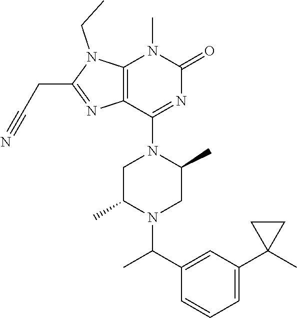 [SMILES: CCn1c(CC#N)nc2c(N3C[C@@H](C)N(C(C)c4cccc(C5(C)CC5)c4)C[C@@H]3C)nc(=O)n(C)c21]

To a solution of 1-(3-(1-methylcyclopropyl)phenyl)ethan-1-ol (120 mg, 0.68 mmol), 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol) and (cyanomethyl) trimethyl phosphonium iodide (218 mg, 0.9 mmol) in CH₃CN (4 mL) was added DIPEA (387 mg, 3 mmol). The mixture was sealed in a bottle and heated at 100° C. for 16 hours. The mixture was cooled to room temperature, diluted with water, extracted with EtOAc (80 mL×2), washed with brine, dried over Na₂SO₄, filtered, and concentrated to dryness. The resulting residue was purified by Prep-HPLC (Method A) to give the titled compound (62 mg, 42%). ¹H NMR (400 MHz, CD₃OD) δ 7.36-7.05 (m, 4H), 6.19-5.65 (m, 1H), 5.31-4.95 (m, 1H), 4.43-4.30 (m, 2H), 4.28-4.16 (m, 1.5H), 3.76 (s, 3H), 3.70-3.40 (m, 2H), 3.35-3.31 (m, 1H), 2.97-2.85 (m, 1.5H), 2.74-2.51 (m, 0.5H), 2.30-2.16 (m, 0.5H), 1.54-1.15 (m, 12H), 1.12-0.51 (m, 7H) ppm. MS: M/e 488 (M+1)⁺.

### Compound A129: 2-(6-((2S,5R)-4-(1-(2-(tert-butyl)pyridin-4-yl)ethyl)-2,5-dimethylpiperazin-1-yl)-9-ethyl-3-methyl-2-oxo-3,9-dihydro-2H-purin-8-yl)acetonitrile

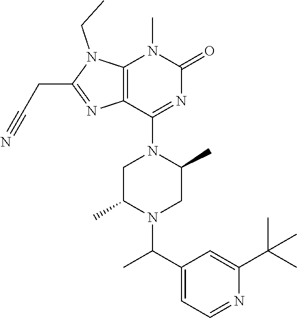 [SMILES: CCn1c(CC#N)nc2c(N3C[C@@H](C)N(C(C)c4ccnc(C(C)(C)C)c4)C[C@@H]3C)nc(=O)n(C)c21]

### Step A: 1-(2-(tert-butyl)pyridin-4-yl)ethan-1-one

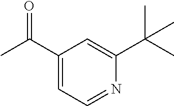

A mixture of 4-bromo-2-(tert-butyl)pyridine (214 mg, 1 mmol), tributyl(1-ethoxyvinyl)stannane (541 mg, 1.5 mmol) and Pd(PPh₃)₂Cl₂ (70 mg, 0.1 mmol) in toluene (5 mL) was stirred at 100° C. under N₂ overnight. To the resulting solution was added TFA (1 mL) in drops and the mixture was stirred at room temperature for 30 minutes. The mixture was diluted with EtOAc (20 mL), treated with saturated Na₂CO₃ aq. to pH ˜8, washed with brine (20 mL×3), dried and concentrated to dryness. The resulting residue was purified by flash column chromatography to give the titled compound (100 mg, 56%). MS: M/e 178 (M+1)⁺.

### Step B: 1-(2-(tert-butyl)pyridin-4-yl)ethan-1-ol

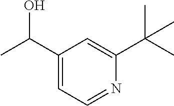

To a solution of 1-(2-(tert-butyl)pyridin-4-yl)ethan-1-one (100 mg, 0.56 mmol) in EtOH (2 mL) was added NaBH₄ (17 mg, 0.45 mmol) at room temperature and the mixture was stirred at room temperature for 1 hour. The mixture was treated with water, extracted with DCM. The combined organic layers were washed with brine, dried over Na₂SO₄ and concentrated to dryness. The resulting residue (80 mg, crude) was used in the next step directly. MS: M/e 180 (M+1)⁺.

### Step C: 2-(6-((2S,5R)-4-(1-(2-(tert-butyl)pyridin-4-yl)ethyl)-2,5-dimethylpiperazin-1-yl)-9-ethyl-3-methyl-2-oxo-3,9-dihydro-2H-purin-8-yl)acetonitrile

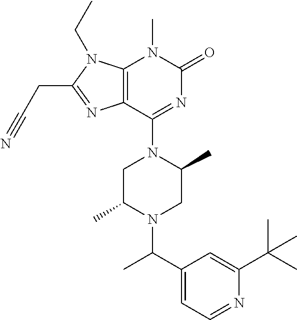 [SMILES: CCn1c(CC#N)nc2c(N3C[C@@H](C)N(C(C)c4ccnc(C(C)(C)C)c4)C[C@@H]3C)nc(=O)n(C)c21]

A mixture of 1-(2-(tert-butyl)pyridin-4-yl)ethan-1-ol (80 mg, 0.45 mmol), 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol), (cyanomethyl)trimethylphosphonium iodide (145 mg, 0.6 mmol) and DIPEA (155 mg, 1.2 mmol) in MeCN (3 mL) was stirred at 100° C. overnight. The reaction mixture was diluted with EtOAc, washed with brine, dried over Na₂SO₄ and concentrated to dryness. The resulting residue was purified by Prep-TLC (DCM/MeOH=13/1) to give the titled compound (19 mg, 13%). ¹H NMR (400 MHz, CD₃OD) δ 8.40 (d, J=8.5 Hz, 1H), 7.57 (d, J=18.7 Hz, 1H), 7.28 (d, J=21.4 Hz, 1H), 5.93 (s, 2H), 4.37 (d, J=6.9 Hz, 2H), 4.23 (s, 2H), 3.89-3.48 (m, 5H), 3.05-2.60 (m, 2H), 2.13 (d, J=11.7 Hz, 1H), 1.58-1.21 (m, 18H), 1.11-0.86 (m, 3H) ppm. MS: M/e 491 (M+1)⁺.

### Compound A134: 2-(3-(1-((2R,5S)-4-(8-(cyanomethyl)-9-ethyl-3-methyl-2-oxo-3,9-dihydro-2H-purin-6-yl)-2,5-dimethylpiperazin-1-yl)ethyl)phenyl)-2-methylpropanenitrile

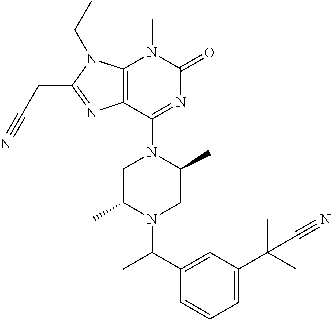 [SMILES: CCn1c(CC#N)nc2c(N3C[C@@H](C)N(C(C)c4cccc(C(C)(C)C#N)c4)C[C@@H]3C)nc(=O)n(C)c21]

### Step A: 2-(3-acetylphenyl)-2-methylpropanenitrile

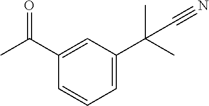

To a solution of 2-(3-bromophenyl)-2-methylpropanenitrile (336 mg, 1.5 mmol) in toluene (6 mL) was added tributyl(1-ethoxyvinyl)stannane (812 mg, 2.25 mmol) and Pd(PPh₃)₂Cl₂ (53 mg, 0.075 mmol). The reaction mixture was protected by N₂ atmosphere and stirred at 100° C. overnight. The mixture was cooled down to RT, added 2N HCl (4 ml) and stirred at room temperature for 30 mins. The mixture was concentrated in vacuo. The residue was added H₂O, extracted with EtOAc (80 mL×2), washed with brine, dried over Na₂SO₄, filtered, and concentrated to dryness. The residue was purified by flash column chromatography to give the titled compound (248 mg, 85%). ¹H NMR (400 MHz, CDCl₃) δ 8.05 (s, 1H), 7.91 (d, J=8.0 Hz, 1H), 7.74 (d, J=7.6 Hz, 1H), 7.52 (t, J=8.0 Hz, 1H), 2.64 (s, 3H), 1.77 (s, 6H) ppm.

### Step B: 2-(3-(1-hydroxyethyl)phenyl)-2-methylpropanenitrile

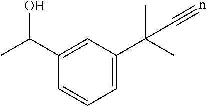

To a solution of 2-(3-acetylphenyl)-2-methylpropanenitrile (93 mg, 0.5 mmol) in MeOH (5 mL) was added NaBH₄ (19 mg, 0.5 mmol) slowly at 0° C. The resulting mixture was stirred at room temperature for 0.5 hour. The reaction mixture was quenched with H₂O (10 mL) and extracted with EtOAc (80 mL), washed with brine, dried over Na₂SO₄, filtered, and concentrated to dryness. The residue was purified by flash column chromatography to give the titled compound (92 mg, 96% yield). ¹H NMR (400 MHz, DMSO-d₆) δ 7.50 (s, 1H), 7.41-7.34 (m, 2H), 7.32-7.27 (m, 1H), 5.24 (s, 1H), 4.80-4.70 (m, 1H), 1.68 (s, 6H), 1.32 (d, J=6.4 Hz, 3H) ppm.

### Step C: 2-(3-(1-((2R,5S)-4-(8-(cyanomethyl)-9-ethyl-3-methyl-2-oxo-3,9-dihydro-2H-purin-6-yl)-2,5-dimethylpiperazin-1-yl)ethyl)phenyl)-2-methylpropanenitrile

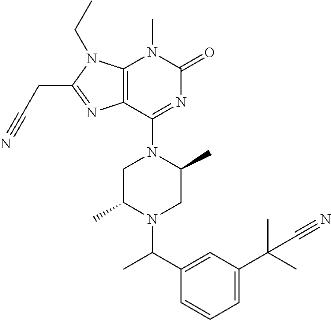 [SMILES: CCn1c(CC#N)nc2c(N3C[C@@H](C)N(C(C)c4cccc(C(C)(C)C#N)c4)C[C@@H]3C)nc(=O)n(C)c21]

To a solution of 2-(3-(1-hydroxyethyl)phenyl)-2-methylpropanenitrile (90 mg, 0.47 mmol), 2-(6-((2S,5R)-2,5-dimethylpiperazin-1-yl)-9-ethyl-3-methyl-2-oxo-3,9-dihydro-2H-purin-8-yl)acetonitrile (100 mg, 0.3 mmol) and (cyanomethyl) trimethyl phosphonium iodide (218 mg, 0.9 mmol) in CH₃CN (4 mL) was added DIPEA (387 mg, 3 mmol). The mixture was sealed in a bottle and heated at 100° C. for 16 hours. The mixture was cooled to room temperature, diluted with water, extracted with EtOAc (80 mL×2), washed with brine, dried over Na₂SO₄, filtered, and concentrated to dryness. The resulting residue was purified by Prep-HPLC (Method A) to give the titled compound (70 mg, 46%). ¹H NMR (400 MHz, CD₃OD) δ 7.67-7.60 (m, 1H), 7.46-7.30 (m, 3H), 6.25-5.62 (m, 1H), 5.31-4.95 (m, 1H), 4.43-4.30 (m, 2H), 4.28-4.16 (m, 2H), 3.85-3.50 (m, 5H), 3.35-3.31 (m, 1H), 3.00-2.85 (m, 1H), 2.75-2.60 (m, 0.5H), 2.26-2.16 (m, 0.5H), 1.74 (s, 6H), 1.54-1.23 (m, 9H), 1.07 (d, J=5.6 Hz, 1.5H), 0.93 (d, J=5.6 Hz, 1.5H) ppm. MS: M/e 501 (M+1)⁺.

### Compound A135, A136: 2-(9-ethyl-6-((2S,5R)-4-(1-(4-fluoro-3-(prop-1-en-2-yl)phenyl)ethyl)-2,5-dimethylpiperazin-1-yl)-3-methyl-2-oxo-3,9-dihydro-2H-purin-8-yl)acetonitrile & 2-(9-ethyl-6-((2S,5R)-4-(1-(4-fluoro-3-(1-methylcyclopropyl)phenyl)ethyl)-2,5-dimethylpiperazin-1-yl)-3-methyl-2-oxo-3,9-dihydro-2H-purin-8-yl)acetonitrile

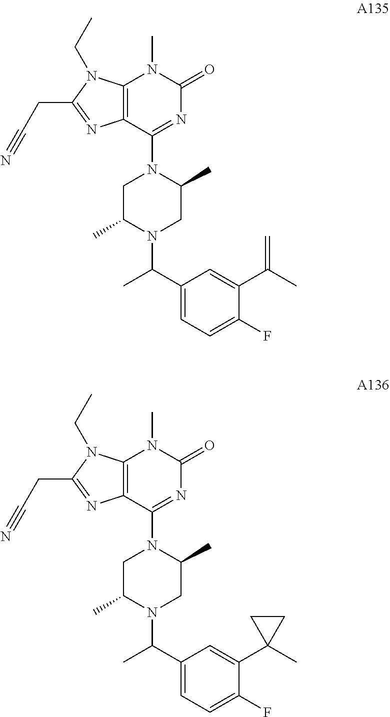 [SMILES: C=C(C)c1cc(C(C)N2C[C@H](C)N(c3nc(=O)n(C)c4c3nc(CC#N)n4CC)C[C@H]2C)ccc1F ; CCn1c(CC#N)nc2c(N3C[C@@H](C)N(C(C)c4ccc(F)c(C5(C)CC5)c4)C[C@@H]3C)nc(=O)n(C)c21]

### Step A: 1-(4-fluoro-3-(prop-1-en-2-yl)phenyl)ethan-1-one

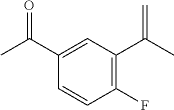

To a solution of 1-(3-bromo-4-fluorophenyl)ethan-1-one (2.17 g, 10 mmol) and K₂CO₃ (2.76 g, 20 mmol) in dioxane/H₂O (10/1, 20 mL) was added 4,4,5,5-tetramethyl-2-(prop-1-en-2-yl)-1,3,2-dioxaborolane (2 g, 12 mmol) and Pd(dppf)₂Cl₂·CH₂Cl₂ (818 mg, 1 mmol). The reaction mixture was protected by N₂ atmosphere and stirred at 100° C. overnight. The mixture was cooled down to RT, diluted with water, extracted with EtOAc (80 mL×2), washed with brine, dried over Na₂SO₄, filtered, and concentrated to dryness. The residue was purified by flash column chromatography to give the titled compound (1.6 g, 89%). ¹H NMR (400 MHz, CDCl₃) δ 8.00-7.81 (m, 2H), 7.15-7.105 (m, 1H), 5.34-5.25 (m, 2H), 2.60 (s, 3H), 2.16 (s, 3H) ppm.

### Step B: 1-(4-fluoro-3-(1-methylcyclopropyl)phenyl)ethan-1-one

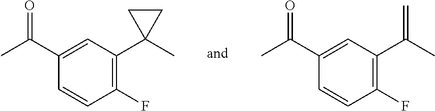

To a solution of Et₂Zn (3 mL, 1 mol/L in hexane) in DCM (8 mL) was added a solution of TFA (684 mg, 6 mmol) in DCM (4 mL) drop wise at ice-bath. After 20 min, a solution of CH₂I₂ (1.57 g, 6 mmol) in DCM (4 mL) was added drop wise. Then 20 min later, a solution of 1-(4-fluoro-3-(prop-1-en-2-yl)phenyl)ethan-1-one (534 mg, 3 mmol) in DCM (4 mL) was added drop wise. Then the reaction mixture was warmed to room temperature overnight. The reaction mixture was diluted with H₂O, extracted with DCM (60 mL), washed with brine, dried over Na₂SO₄, filtered, and concentrated to dryness. The residue was purified by flash column chromatography to give the titled compound (440 mg, a mixture of 1-(4-fluoro-3-(prop-1-en-2-yl)phenyl)ethan-1-one and 1-(4-fluoro-3-(1-methylcyclopropyl)phenyl)ethan-1-one, ˜3/1). ¹H NMR (400 MHz, CDCl₃) δ 8.01-7.75 (m, 2H), 7.10-7.00 (m, 1H), 2.60 (s, 3H), 1.36 (s, 3H), 0.91-0.62 (m, 4H) ppm.

### Step C: 1-(4-fluoro-3-(prop-1-en-2-yl)phenyl)ethan-1-ol and 1-(4-fluoro-3-(1-methylcyclopropyl) phenyl)ethan-1-ol

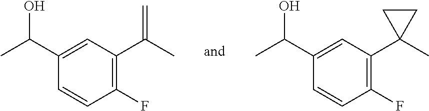

To a solution of 1-(4-fluoro-3-(prop-1-en-2-yl)phenyl)ethan-1-one and 1-(4-fluoro-3-(1-methylcyclopropyl)phenyl)ethan-1-one (440 mg, 2.29 mmol) in MeOH (6 mL) was added NaBH₄ (87 mg, 2.29 mmol) slowly at 0° C. The resulting mixture was stirred at room temperature for 0.5 hour. The reaction mixture was quenched with H₂O (10 mL) and extracted with EtOAc (80 mL), washed with brine, dried over Na₂SO₄, filtered, and concentrated to dryness. The residue was purified by flash column chromatography to give the desired mixture (260 mg, 59% yield). MS: M/e 181 and 195 (M+1)⁺.

### Step D: 2-(9-ethyl-6-((2S,5R)-4-(1-(4-fluoro-3-(prop-1-en-2-yl)phenyl)ethyl)-2,5-dimethylpiperazin-1-yl)-3-methyl-2-oxo-3,9-dihydro-2H-purin-8-yl)acetonitrile and 2-(9-ethyl-6-((2S,5R)-4-(1-(4-fluoro-3-(1-methylcyclopropyl)phenyl)ethyl)-2,5-dimethylpiperazin-1-yl)-3-methyl-2-oxo-3,9-dihydro-2H-purin-8-yl)acetonitrile

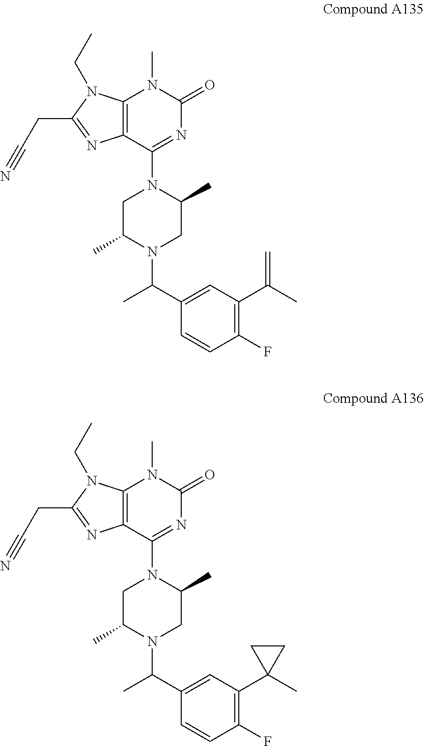 [SMILES: C=C(C)c1cc(C(C)N2C[C@H](C)N(c3nc(=O)n(C)c4c3nc(CC#N)n4CC)C[C@H]2C)ccc1F ; CCn1c(CC#N)nc2c(N3C[C@@H](C)N(C(C)c4ccc(F)c(C5(C)CC5)c4)C[C@@H]3C)nc(=O)n(C)c21]

To a solution of 1-(4-fluoro-3-(prop-1-en-2-yl)phenyl)ethan-1-ol and 1-(4-fluoro-3-(1-methylcyclopropyl)phenyl)ethan-1-ol (260 mg, 1.36 mmol), 2-(6-((2S,5R)-2,5-dimethylpiperazin-1-yl)-9-ethyl-3-methyl-2-oxo-3,9-dihydro-2H-purin-8-yl)acetonitrile (320 mg, 1 mmol) and (cyanomethyl) trimethyl phosphonium iodide (730 mg, 3 mmol) in CH₃CN (5 mL) was added DIPEA (1.29 g, 10 mmol). The mixture was sealed in a bottle and heated at 100° C. for 16 hours. The mixture was cooled to room temperature, diluted with water, extracted with EtOAc (80 mL×2), washed with brine, dried over Na₂SO₄, filtered, and concentrated to dryness. The resulting residue was purified and separated into Compound A135 (40 mg) and A136 (80 mg) by Prep-HPLC (Method A).

Compound A135: ¹H NMR (400 MHz, CD₃OD) δ 7.43-7.24 (m, 2H), 7.09-6.96 (m, 1H), 6.19-5.65 (m, 1H), 5.31-4.95 (m, 3H), 4.43-4.30 (m, 2H), 4.28-4.16 (m, 1.5H), 3.76 (s, 3H), 3.79-3.42 (m, 2H), 3.35-3.31 (m, 1H), 3.00-2.85 (m, 1.5H), 2.75-2.59 (m, 0.5H), 2.28-2.19 (m, 0.5H), 2.16-2.10 (m, 3H), 1.55-1.21 (m, 9H), 1.05 (d, J=6.0 Hz, 1.5H), 0.93 (d, J=6.4 Hz, 1.5H) ppm. MS: M/e 492 (M+1)⁺.

Compound A136: ¹H NMR (400 MHz, CD₃OD) δ 7.44-7.13 (m, 2H), 7.06-6.92 (m, 1H), 6.19-5.65 (m, 1H), 5.31-4.95 (m, 1H), 4.43-4.30 (m, 2H), 4.28-4.16 (m, 1.5H), 3.76 (s, 3H), 3.79-3.40 (m, 2H), 3.35-3.31 (m, 1H), 3.00-2.85 (m, 1.5H), 2.75-2.61 (m, 0.5H), 2.28-2.17 (m, 0.5H), 1.54-1.19 (m, 12H), 1.06 (d, J=6.8 Hz, 1.5H), 0.94 (d, J=6.4 Hz, 1.5H), 0.84-0.65 (m, 4H) ppm. MS: M/e 506 (M+1)⁺.

### Compound A137: 2-(6-((2S,5R)-2,5-dimethyl-4-(1-(3-methyl-4-(trifluoromethyl)phenyl) ethyl)piperazin-1-yl)-9-ethyl-3-methyl-2-oxo-3,9-dihydro-2H-purin-8-yl)acetonitrile

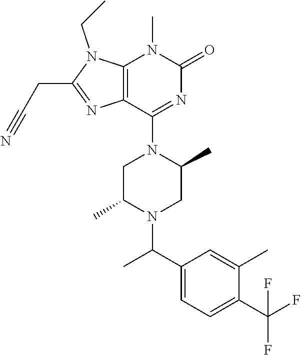 [SMILES: CCn1c(CC#N)nc2c(N3C[C@@H](C)N(C(C)c4ccc(C(F)(F)F)c(C)c4)C[C@@H]3C)nc(=O)n(C)c21]

### Step A: 1-(3-methyl-4-(trifluoromethyl)phenyl)ethan-1-ol

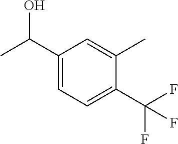

To a −70° C. solution of 4-bromo-2-methyl-1-(trifluoromethyl)benzene (1.0 g, 4.2 mmol) in THE (10 mL) was added n-BuLi (1.6 M, 3.2 mL, 5.1 mmol) in drops under N₂. After stirring for 20 minutes at −70° C., a solution of acetaldehyde (440 mg, 10 mmol) in THE (2 mL) was added. Then the mixture was allowed warm to room temperature and stirred for 16 hours. Aqueous solution of NH₄Cl (20 mL) was added and the mixture was extracted with EtOAc (10 mL×3), the organics were combined and washed with brine (10 mL×2), dried over Na₂SO₄, filtered and concentrated to dryness. The resulting residue was purified by column chromatography to give the titled compound (678 mg, 79%). ¹H NMR (400 MHz, DMSO-d6) δ 7.60 (d, J=8.0 Hz, 1H), 7.39 (s, 1H), 7.34 (d, J=8.0 Hz, 1H), 5.32 (d, J=4.4 Hz, 1H), 4.85-4.64 (m, 1H), 2.43 (s, 3H), 1.32 (d, J=6.4 Hz, 3H) ppm.

### Step B: 2-(6-((2S,5R)-2,5-dimethyl-4-(1-(3-methyl-4-(trifluoromethyl)phenyl) ethyl)piperazin-1-yl)-9-ethyl-3-methyl-2-oxo-3,9-dihydro-2H-purin-8-yl) acetonitrile

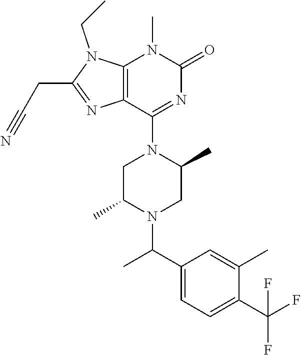 [SMILES: CCn1c(CC#N)nc2c(N3C[C@@H](C)N(C(C)c4ccc(C(F)(F)F)c(C)c4)C[C@@H]3C)nc(=O)n(C)c21]

To a mixture of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol), 1-(3-methyl-4-(trifluoromethyl)phenyl)ethan-1-ol (65 mg, 0.32 mmol) and (cyanomethyl)trimethylphosphonium iodide (120 mg, 0.50 mmol) in CH₃CN (1 mL) was added DIPEA (155 mg, 1.2 mmol). The mixture was stirred at 100° C. in a sealed tube for 16 hours. The mixture was cooled and diluted with EtOAc (20 mL), washed with brine (10 mL×3), dried over Na₂SO₄, filtered and concentrated under reduced pressure. The resulting residue was purified by flash column chromatography and Prep-HPLC (Method A) to give the titled compound (36 mg, 46%). ¹H NMR (400 MHz, CD₃OD) δ 7.64-7.52 (m, 1H), 7.48-7.30 (m, 2H), 6.29-4.87 (m, 2H), 4.68-4.04 (m, 4H), 3.76 (s, 3H), 3.73-3.44 (m, 2H), 3.08-2.03 (m, 6H), 1.54-1.17 (m, 9H), 1.11-0.84 (m, 3H) ppm. MS: M/e 516 (M+1)⁺.

### Compound A144: 2-(6-((2S,5R)-2,5-dimethyl-4-(1-(2-phenylpyridin-4-yl)ethyl)piperazin-1-yl)-9-ethyl-3-methyl-2-oxo-3,9-dihydro-2H-purin-8-yl)acetonitrile

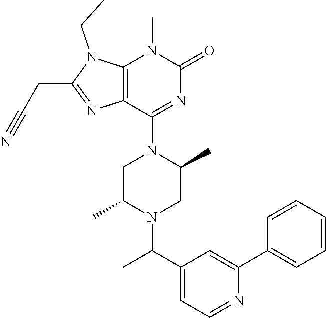 [SMILES: CCn1c(CC#N)nc2c(N3C[C@@H](C)N(C(C)c4ccnc(-c5ccccc5)c4)C[C@@H]3C)nc(=O)n(C)c21]

### Step A: 4-bromo-2-phenylpyridine

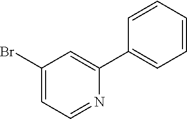 [SMILES: Brc1ccnc(-c2ccccc2)c1]

To a solution of 2,4-dibromopyridine (1.18 g, 5 mol) in MeCN (10 mL) was added 4,4,5,5-tetramethyl-2-phenyl-1,3,2-dioxaborolane (1 g, 5 mol), Pd(OAc)₂ (56 mg, 0.25 mmol), PPh₃ (262 mg, 1 mmol) and KOH (560 mg, 10 mmol). The reaction mixture was protected by N₂ atmosphere and stirred at 70° C. for 16 hrs. The mixture was cooled down to RT and added H₂O (20 ml), The mixture was extracted with EtOAc (20 mL×3), then concentrated by using a rotary evaporator to give a residue. The resulting residue was purified by flash column chromatography to give the titled compound (800 mg, 69%). MS: M/e 234 (M+1)⁺.

### Step B: 1-(2-phenylpyridin-4-yl)ethan-1-one

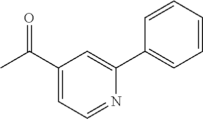

To a solution of 4-bromo-2-phenylpyridine (232 mg, 1 mol) in toluene (10 mL) was added Pd(PPh₃)₂Cl₂ (70 mg, 0.1 mol) and tributyl(1-ethoxyvinyl)stannane (541 mg, 1.5 mmol). The reaction mixture was protected by N₂ atmosphere and stirred at 90° C. for 16 hrs. The mixture was added 4N HCl in dioxane (0.5 ml) and stirred at RT for 15 mins. The mixture was added saturated NaHCO₃ aqueous solution (10 ml) and extracted with EtOAc (20 mL×3). The organic phase was dried with Na₂SO₄ and concentrated by using a rotary evaporator to give a residue. The resulting residue was purified by flash column chromatography to give the titled compound (40 mg, 20%). MS: M/e 198 (M+1)⁺.

### Step C: 1-(2-phenylpyridin-4-yl)ethan-1-ol

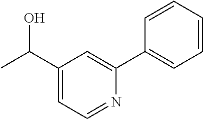

To a solution of 1-(2-phenylpyridin-4-yl)ethan-1-one (40 mg, 0.2 mol) in MeOH was added NaBH₄ (38 mg, 1 mmol). The reaction mixture was stirred at RT for 15 mins. The mixture was added H₂O (20 ml) and extracted with DCM (20 mL×3). The organic phase was dried with Na₂SO₄ and concentrated by using a rotary evaporator to give a residue. The resulting residue was purified by flash column chromatography to give the titled compound (40 mg, 100%). MS: M/e 200 (M+1)⁺.

### Step D: 2-(6-((2S,5R)-2,5-dimethyl-4-(1-(2-phenylpyridin-4-yl)ethyl)piperazin-1-yl)-9-ethyl-3-methyl-2-oxo-3,9-dihydro-2H-purin-8-yl)acetonitrile

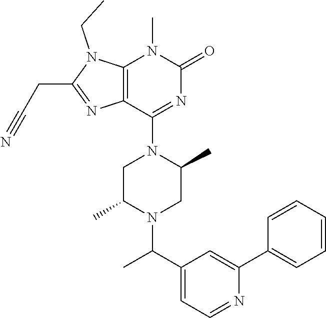 [SMILES: CCn1c(CC#N)nc2c(N3C[C@@H](C)N(C(C)c4ccnc(-c5ccccc5)c4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl) acetonitrile (30 mg, 0.1 mmol), 1-(2-phenylpyridin-4-yl)ethan-1-ol (40 mg, 0.2 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EtOAc (20 mL×3). The combined organic layers were washed with brine, dried over Na₂SO₄, filtered, and concentrated to dryness. The resulting residue was purified by Prep-HPLC (Method A) to give the titled compound (5 mg, 9%). ¹H NMR (400 MHz, CD₃OD) δ 8.55 (dd, J=11.8, 5.1 Hz, 1H), 7.92 (d, J=8.0 Hz, 3H), 7.48 (ddd, J=21.5, 11.8, 6.2 Hz, 4H), 5.95 (m, 1H), 5.09 (m, 1H), 4.36 (m, 2H), 4.22 (s, 2H), 3.78 (m, 4H), 3.63 (d, J=6.2 Hz, 1H), 2.89 (m, 2H), 2.75 (s, 0.5H), 2.23 (d, J=12.2 Hz, 0.5H), 1.46 (t, J=7.2 Hz, 4H), 1.38 (m, 5H), 1.08 (d, J=6.4 Hz, 1.5H), 0.97 (d, J=6.4 Hz, 1.5H) ppm. MS: M/e 507 (M+1)⁺.

### Compound A149: 2-(6-((2S,5R)-2,5-diethyl-4-(1-(4-(trifluoromethyl)phenyl) ethyl)piperazin-1-yl)-3,9-dimethyl-2-oxo-3,9-dihydro-2H-purin-8-yl)acetonitrile

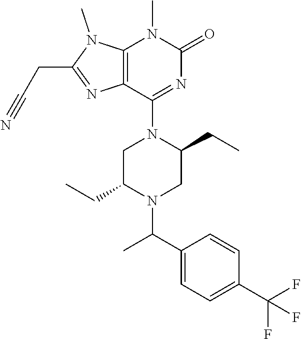 [SMILES: CC[C@H]1CN(C(C)c2ccc(C(F)(F)F)cc2)[C@H](CC)CN1c1nc(=O)n(C)c2c1nc(CC#N)n2C]

### Step A: tert-butyl (2R,5S)-2,5-diethyl-4-(1-methyl-6-(methylamino)-5-nitro-2-oxo-1,2-dihydropyrimidin-4-yl)piperazine-1-carboxylate

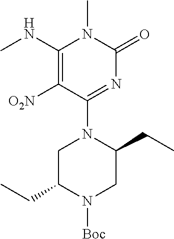 [SMILES: CC[C@@H]1CN(c2nc(=O)n(C)c(NC)c2[N+](=O)[O-])[C@@H](CC)CN1C(=O)OC(C)(C)C]

To a stirred mixture of 4-hydroxy-1-methyl-6-(methylamino)-5-nitropyrimidin-2 (1H)-one (600 mg, 3 mmol), tert-butyl (2R,5S)-2,5-diethylpiperazine-1-carboxylate (871.2 mg, 3.6 mmol) and BOP (2 g, 4.5 mmol) in DMF (10 mL) was added DBU (912 mg, 6 mmol). After the addition, the reaction mixture was stirred at 70° C. overnight. The reaction mixture was concentrated to give the residue, which was treated with H₂O (50 mL) and extracted with EtOAc (60 mL×3). The combined organic layers were washed with brine, dried over Na₂SO₄, concentrated to dryness. The resulting residue was purified by flash column chromatography to give the titled compound (1.0 g, 82%). MS: M/e 425 (M+1)⁺.

### Step B: tert-butyl (2R,5S)-4-(5-amino-1-methyl-6-(methylamino)-2-oxo-1,2-dihydropyrimidin-4-yl)-2,5-diethylpiperazine-1-carboxylate

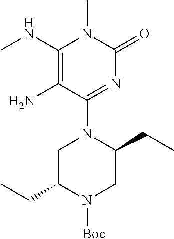 [SMILES: CC[C@@H]1CN(c2nc(=O)n(C)c(NC)c2N)[C@@H](CC)CN1C(=O)OC(C)(C)C]

To a stirred solution of tert-butyl (2R,5S)-2,5-diethyl-4-(1-methyl-6-(methylamino)-5-nitro-2-oxo-1,2-dihydropyrimidin-4-yl)piperazine-1-carboxylate (1.04 g, 2.45 mmol) in CH₂Cl₂/MeOH (8 mL/8 mL) was added NiCl₂ (637 mg, 4.9 mmol), followed by NaBH₄ (186.2 mg, 4.9 mmol) at 0° C. After stirred for 20 min, the reaction was quenched with aq.NH₄Cl, extracted with EtOAc (20 mL×4). The combined organic layers were washed with brine, dried over Na₂SO₄, concentrated to give the titled compound (960 mg, 100%). MS: M/e 395 (M+1)⁺.

### Step C: tert-butyl (2R,5S)-4-(5-(2-cyanoacetamido)-1-methyl-6-(methylamino)-2-oxo-1,2-dihydropyrimidin-4-yl)-2,5-diethylpiperazine-1-carboxylate

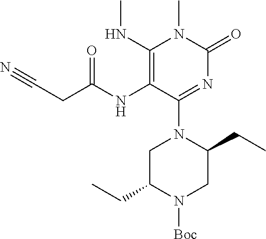 [SMILES: CC[C@@H]1CN(c2nc(=O)n(C)c(NC)c2NC(=O)CC#N)[C@@H](CC)CN1C(=O)OC(C)(C)C]

A mixture of tert-butyl (2R,5S)-4-(5-amino-1-methyl-6-(methylamino)-2-oxo-1,2-dihydropyrimidin-4-yl)-2,5-diethylpiperazine-1-carboxylate (500 mg, 1.27 mmol), 2-cyanoacetic acid (129 mg, 1.52 mmol), HATU (580 mg, 1.52 mmol) and DIPEA (327 mg, 2.54 mmol) in CH₂Cl₂ (10 mL) was stirred overnight. The reaction mixture was diluted with CH₂Cl₂ (10 mL), washed with brine, dried over Na₂SO₄, concentrated to dryness. The resulting residue was purified by flash column chromatography to give the titled compound (330 mg, 56%). MS: M/e 462 (M+1)⁺.

### Step D: 2-(6-((2S,5R)-2,5-diethylpiperazin-1-yl)-3,9-dimethyl-2-oxo-3,9-dihydro-2H-purin-8-yl)acetonitrile

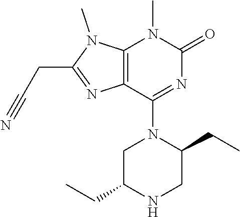 [SMILES: CC[C@@H]1CN(c2nc(=O)n(C)c3c2nc(CC#N)n3C)[C@@H](CC)CN1]

A solution of tert-butyl (2R,5S)-4-(5-(2-cyanoacetamido)-1-methyl-6-(methylamino)-2-oxo-1,2-dihydropyrimidin-4-yl)-2,5-diethylpiperazine-1-carboxylate (330 mg, 0.71 mmol) in AcOH (5 mL) was stirred at 100° C. overnight. The reaction mixture was concentrated to give the residue, which was dissolved in CH₂Cl₂ (10 mL) and EtOAc/HCl(g) (4.0 M, 5 mL) was added and stirred for 3 hours. The reaction mixture was concentrated, H₂O (1 mL) was added, then basified to pH=9˜10 with Na₂CO₃, concentrated to dryness. The resulting residue was purified by flash column chromatography to give the titled compound (109 mg, 45%). MS: M/e 344 (M+1)⁺.

### Step E: 2-(6-((2S,5R)-2,5-diethyl-4-(1-(4-(trifluoromethyl)phenyl)ethyl)piperazin-1-yl)-3,9-dimethyl-2-oxo-3,9-dihydro-2H-purin-8-yl)acetonitrile

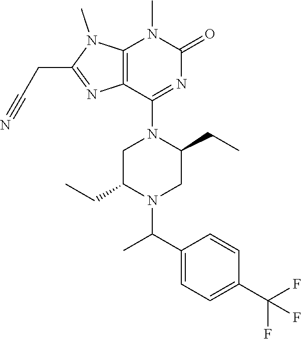 [SMILES: CC[C@H]1CN(C(C)c2ccc(C(F)(F)F)cc2)[C@H](CC)CN1c1nc(=O)n(C)c2c1nc(CC#N)n2C]

A mixture of 2-(6-((2S,5R)-2,5-diethylpiperazin-1-yl)-3,9-dimethyl-2-oxo-3,9-dihydro-2H-purin-8-yl)acetonitrile (34.3 mg, 0.1 mmol), 1-(4-(trifluoromethyl)phenyl)ethan-1-ol (28.5 mg, 0.15 mmol), (cyanomethyl)trimethylphosphonium iodide (72.9 mg, 0.3 mmol) and DIPEA (64.5 mg, 0.5 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to dryness. The resulting residue was purified and separated into Compound A149a (3 mg) and Compound A149b (5 mg) by Prep-HPLC (Method A).

Compound A149a (the earlier peak): ¹H NMR (400 MHz, CD₃OD) δ 7.67-7.54 (m, 4H), 6.02-5.08 (m, 2H), 3.92 (d, J=8.8 Hz, 3H), 3.85-3.79 (m, 1H), 3.78 (s, 3H), 3.48-3.36 (m, 1H), 3.05-2.65 (m, 3H), 2.42-1.91 (m, 4H), 1.53-1.26 (m, 2H), 1.33 (d, J=6.4 Hz, 3H), 0.93 (t, J=7.2 Hz, 3H), 0.75-0.63 (m, 3H) ppm. MS: M/e 516 (M+1)⁺.

Compound A149b (the later peak): ¹H NMR (400 MHz, CD₃OD) δ 7.64-7.56 (m, 4H), 6.19-5.08 (m, 2H), 4.28-4.12 (m, 1H), 3.92 (s, 3H), 3.78 (s, 3H), 3.70-3.59 (m, 1.5H), 3.20-3.11 (m, 1.5H), 2.75-2.51 (m, 1H), 2.29-2.21 (m, 1H), 1.94-1.45 (m, 5H), 1.31 (d, J=6.4 Hz, 3H), 1.06-0.96 (m, 3H), 0.66 (t, J=7.2 Hz, 3H) ppm. MS: M/e 516 (M+1)⁺.

### Compound A150: 2-(6-((2S,5R)-2,5-dimethyl-4-(1-(4-(trifluoromethyl)phenyl)ethyl)piperazin-1-yl)-3-methyl-2-oxo-3,9-dihydro-2H-purin-8-yl)acetonitrile

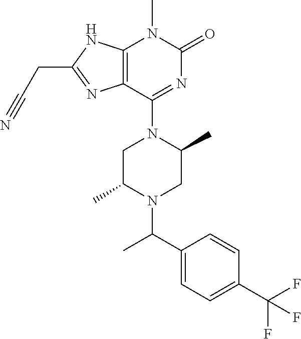 [SMILES: CC(c1ccc(C(F)(F)F)cc1)N1C[C@H](C)N(c2nc(=O)n(C)c3[nH]c(CC#N)nc23)C[C@H]1C]

### Step A: 6-chloro-1-methyl-5-nitropyrimidine-2,4 (1H,3H)-dione

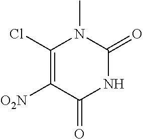

6-chloro-1-methylpyrimidine-2,4 (1H,3H)-dione (4.8 g, 30 mmol) was dissolved in conc. H₂SO₄ (6 mL), then fluming HNO₃ (6 mL) was added dropwise at 0° C. After the addition, the reaction mixture was stirred for 30 min. The reaction mixture was added to ice-H₂O dropwise, then filtered. The cake was collected, dried to give the titled compound (3.8 g, 62%). MS: M/e 206 (M+1)⁺.

### Step B: 6-amino-1-methyl-5-nitropyrimidine-2,4 (1H,3H)-dione

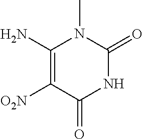

To a stirred suspension of 6-chloro-1-methyl-5-nitropyrimidine-2,4 (1H,3H)-dione (2.5 g, 12.2 mmol) in EtOH (20 mL) was added NH₃·H₂O (˜2 mL, 24.2 mmol). Then the reaction mixture was stirred for 4 hours at RT. The reaction mixture was filtered and the cake was collected, which was suspended in H₂O (30 mL) and acidified to pH=3˜4 with aq.citric acid, the reaction mixture was filtered again and the cake was collected, dried to give the titled compound (1.8 g, 79%). MS: M/e 187 (M+1)⁺.

### Step C: tert-butyl (2R,5S)-4-(6-amino-1-methyl-5-nitro-2-oxo-1,2-dihydropyrimidin-4-yl)-2,5-dimethylpiperazine-1-carboxylate

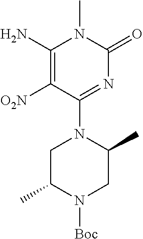 [SMILES: C[C@@H]1CN(c2nc(=O)n(C)c(N)c2[N+](=O)[O-])[C@@H](C)CN1C(=O)OC(C)(C)C]

To a stirred mixture of 6-amino-1-methyl-5-nitropyrimidine-2,4 (1H,3H)-dione (617 mg, 3.32 mmol), tert-butyl (2R,5S)-2,5-dimethylpiperazine-1-carboxylate (852 mg, 4 mmol) and BOP (2.2 g, 4.98 mmol) in DMF (10 mL) was added DBU (1 g, 6.64 mmol). After the addition, the reaction mixture was stirred at 70° C. overnight. The reaction mixture was concentrated to give the residue, which was treated with H₂O (50 mL) and extracted with EtOAc (60 mL×3). The combined organic layers were washed with brine, dried over Na₂SO₄, concentrated to dryness. The resulting residue was purified by flash column chromatography to give the titled crude compound (800 mg). MS: M/e 383 (M+1)⁺.

### Step D: 6-amino-4-((2S,5R)-2,5-dimethylpiperazin-1-yl)-1-methyl-5-nitropyrimidin-2 (1H)-one

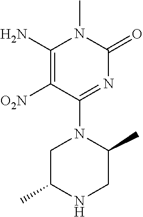 [SMILES: C[C@@H]1CN(c2nc(=O)n(C)c(N)c2[N+](=O)[O-])[C@@H](C)CN1]

To a stirred solution of tert-butyl (2R,5S)-4-(6-amino-1-methyl-5-nitro-2-oxo-1,2-dihydropyrimidin-4-yl)-2,5-dimethylpiperazine-1-carboxylate (800 mg, 2.09 mmol) in CH₂Cl₂ (5 mL) was added EtOAc/HCl (g) (4.0 M, 10 mL). After then, the reaction mixture was stirred for 3 hours. The reaction mixture was filtered and the cake was collected, basified to pH=9˜10 with aq.Na₂CO₃, then concentrated to dryness. The resulting residue was purified by flash column chromatography to give the titled compound (260 mg, 28% over 2 steps). MS: M/e 283 (M+1)⁺.

### Step E: 6-amino-4-((2S,5R)-2,5-dimethyl-4-(1-(4-(trifluoromethyl)phenyl)ethyl)piperazin-1-yl)-1-methyl-5-nitropyrimidin-2 (1H)-one

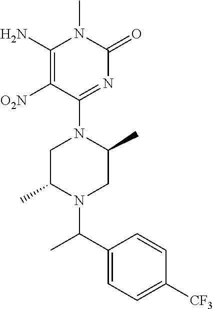 [SMILES: CC(c1ccc(C(F)(F)F)cc1)N1C[C@H](C)N(c2nc(=O)n(C)c(N)c2[N+](=O)[O-])C[C@H]1C]

A mixture of 6-amino-4-((2S,5R)-2,5-dimethylpiperazin-1-yl)-1-methyl-5-nitropyrimidin-2 (1H)-one (100 mg, 0.355 mmol), 1-(4-(trifluoromethyl)phenyl)ethan-1-ol (101 mg, 0.53 mmol), (cyanomethyl)trimethylphosphonium iodide (259 mg, 1.065 mmol) and DIPEA (228 mg, 1.775 mmol) in CH₃CN (5 mL) was stirred overnight at 100° C. in a sealed tube. The reaction mixture was diluted with EtOAc (20 mL), washed with H₂O, brine, dried over Na₂SO₄, concentrated to dryness. The resulting residue was purified by flash column chromatography to give the titled compound (106 mg, 66%). MS: M/e 455 (M+1)⁺.

### Step F: 5,6-diamino-4-((2S,5R)-2,5-dimethyl-4-(1-(4-(trifluoromethyl)phenyl)ethyl)piperazin-1-yl)-1-methylpyrimidin-2 (1H)-one

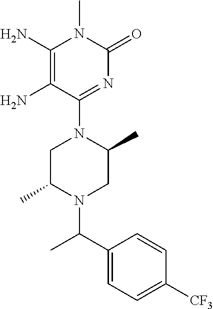 [SMILES: CC(c1ccc(C(F)(F)F)cc1)N1C[C@H](C)N(c2nc(=O)n(C)c(N)c2N)C[C@H]1C]

To a stirred solution of 6-amino-4-((2S,5R)-2,5-dimethyl-4-(1-(4-(trifluoromethyl)phenyl) ethyl)piperazin-1-yl)-1-methyl-5-nitropyrimidin-2 (1H)-one (30 mg, 0.066 mmol) in CH₂Cl₂/MeOH (4 mL/4 mL) was added NiCl₂ (17.2 mg, 0.132 mmol), followed by NaBH₄ (5 mg, 0.132 mmol) at 0° C. After stirred for 20 min, the reaction was quenched with aq.NH₄Cl, extracted with EtOAc (10 mL×2). The combined organic layers were washed with brine, dried over Na₂SO₄, concentrated to give the titled compound, which was directly used to the next step. MS: M/e 425 (M+1)⁺.

### Step G: N-(6-amino-4-((2S,5R)-2,5-dimethyl-4-(1-(4-(trifluoromethyl)phenyl)ethyl)piperazin-1-yl)-1-methyl-2-oxo-1,2-dihydropyrimidin-5-yl)-2-cyanoacetamide

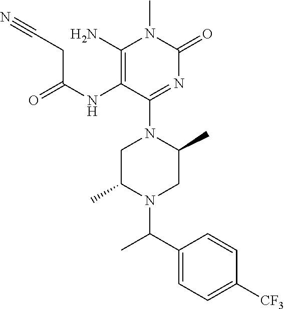 [SMILES: CC(c1ccc(C(F)(F)F)cc1)N1C[C@H](C)N(c2nc(=O)n(C)c(N)c2NC(=O)CC#N)C[C@H]1C]

A mixture of 5,6-diamino-4-((2S,5R)-2,5-dimethyl-4-(1-(4-(trifluoromethyl)phenyl)ethyl)piperazin-1-yl)-1-methylpyrimidin-2 (1H)-one (0.088 mmol), 2-cyanoacetic acid (7.48 mg, 0.088 mmol), HATU (40 mg, 0.105 mmol) and DIPEA (22.7 mg, 0.176 mmol) in CH₂Cl₂ (5 mL) was stirred overnight. The reaction mixture was diluted with CH₂Cl₂ (10 mL), washed with brine, dried over Na₂SO₄, concentrated and purified by Pre-TLC (CH₂Cl₂/MeOH=10:1) to give the titled compound (11 mg, 25%). MS: M/e 492 (M+1)⁺.

### Step H: 2-(6-((2S,5R)-2,5-dimethyl-4-(1-(4-(trifluoromethyl)phenyl)ethyl)piperazin-1-yl)-3-methyl-2-oxo-3,9-dihydro-2H-purin-8-yl)acetonitrile

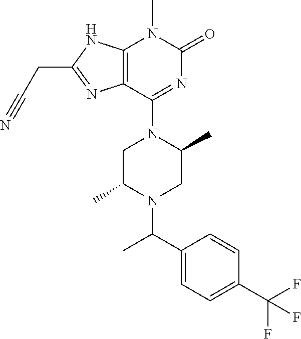 [SMILES: CC(c1ccc(C(F)(F)F)cc1)N1C[C@H](C)N(c2nc(=O)n(C)c3[nH]c(CC#N)nc23)C[C@H]1C]

A solution of N-(6-amino-4-((2S,5R)-2,5-dimethyl-4-(1-(4-(trifluoromethyl)phenyl)ethyl)piperazin-1-yl)-1-methyl-2-oxo-1,2-dihydropyrimidin-5-yl)-2-cyanoacetamide (11 mg, 0.022 mmol) in AcOH (3 mL) was stirred at 80° C. overnight. The reaction mixture was concentrated and purified by Prep-HPLC (Method A) to give the titled compound (4 mg). ¹H NMR (400 MHz, DMSO-d6) δ 7.75-7.58 (m, 4H), 5.92-4.31 (m, 2H), 4.14-4.02 (m, 2H), 3.76-3.50 (m, 2H), 3.37 (s, 3H), 2.88-2.57 (m, 2.5H), 2.06 (d, J=13.2 Hz, 0.5H), 1.46-1.17 (m, 6H), 1.03-0.81 (m, 3H) ppm. MS: M/e 474 (M+1)⁺.

### Compound A152: 8-(but-2-yn-1-yl)-6-((2S,5R)-2,5-diethyl-4-(1-(4-(trifluoromethyl)phenyl) ethyl)piperazin-1-yl)-3,9-dimethyl-3,9-dihydro-2H-purin-2-one

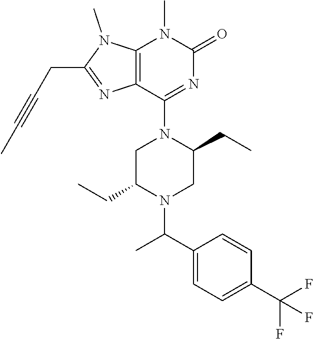 [SMILES: CC#CCc1nc2c(N3C[C@@H](CC)N(C(C)c4ccc(C(F)(F)F)cc4)C[C@@H]3CC)nc(=O)n(C)c2n1C]

### Step A: tert-butyl (2R,5S)-2,5-diethyl-4-(1-methyl-6-(methylamino)-2-oxo-5-(pent-3-ynamido)-1,2-dihydropyrimidin-4-yl)piperazine-1-carboxylate

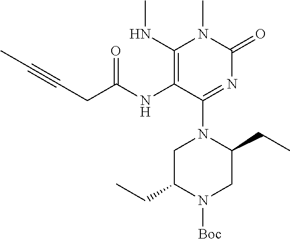 [SMILES: CC#CCC(=O)Nc1c(N2C[C@@H](CC)N(C(=O)OC(C)(C)C)C[C@@H]2CC)nc(=O)n(C)c1NC]

A mixture of tert-butyl (2R,5S)-4-(5-amino-1-methyl-6-(methylamino)-2-oxo-1,2-dihydropyrimidin-4-yl)-2,5-diethylpiperazine-1-carboxylate (460 mg, 1.17 mmol), pent-3-ynoic acid (137 mg, 1.4 mmol), HATU (535 mg, 1.4 mmol) and DIPEA (302 mg, 2.34 mmol) in CH₂Cl₂ (10 mL) was stirred overnight. The reaction mixture was diluted with CH₂Cl₂ (10 mL), washed with brine, dried over Na₂SO₄, concentrated to dryness. The resulting residue was purified by flash column chromatography to give the titled compound (153 mg, 27.5%) as a crude product, which was directly used to the next step. MS: M/e 475 (M+1)⁺.

### Step B: tert-butyl (2R,5S)-4-(8-(but-2-yn-1-yl)-3,9-dimethyl-2-oxo-3,9-dihydro-2H-purin-6-yl)-2,5-diethylpiperazine-1-carboxylate

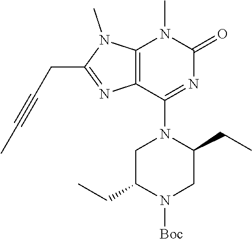 [SMILES: CC#CCc1nc2c(N3C[C@@H](CC)N(C(=O)OC(C)(C)C)C[C@@H]3CC)nc(=O)n(C)c2n1C]

A solution of tert-butyl (2R,5S)-2,5-diethyl-4-(1-methyl-6-(methylamino)-2-oxo-5-(pent-3-ynamido)-1,2-dihydropyrimidin-4-yl)piperazine-1-carboxylate (150 mg, 0.32 mmol) in AcOH (4 mL) was stirred at 80° C. for 6 hours. The reaction mixture was concentrated to give the residue, which was dissolved in CH₂Cl₂ (10 mL) and EtOAc/HCl(g) (4.0 M, 5 mL) was added and stirred for 3 hours. The reaction mixture was concentrated. The resulting residue was purified by Pre-TLC (CH₂Cl₂/MeOH=10:1) to give the titled compound (40 mg, 27%). MS: M/e 456 (M+1)⁺.

### Step C: 8-(but-2-yn-1-yl)-6-((2S,5R)-2,5-diethylpiperazin-1-yl)-3,9-dimethyl-3,9-dihydro-2H-purin-2-one

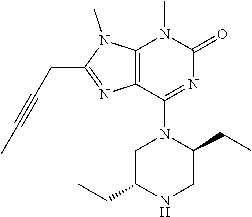 [SMILES: CC#CCc1nc2c(N3C[C@@H](CC)NC[C@@H]3CC)nc(=O)n(C)c2n1C]

To a stirred solution of tert-butyl (2R,5S)-4-(8-(but-2-yn-1-yl)-3,9-dimethyl-2-oxo-3,9-dihydro-2H-purin-6-yl)-2,5-diethylpiperazine-1-carboxylate (40 mg, 0.088 mmol) in CH₂Cl₂ (4 mL) was added EtOAc/HCl(g) (4.0 M, 2 mL) and stirred for 3 hours. The reaction mixture was concentrated. The resulting residue was basified with MeOH/NH₃ (g) and purified by Pre-TLC (CH₂Cl₂/MeOH(NH₃)=10:1) to give the titled compound (20 mg, 64%). MS: M/e 357 (M+1)⁺.

### Step D: 8-(but-2-yn-1-yl)-6-((2S,5R)-2,5-diethyl-4-(1-(4-(trifluoromethyl)phenyl)ethyl) piperazin-1-yl)-3,9-dimethyl-3,9-dihydro-2H-purin-2-one

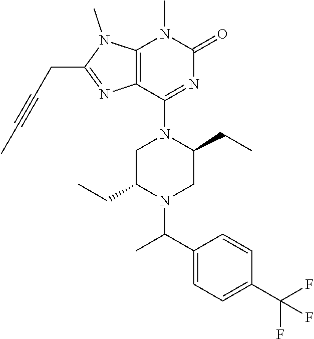 [SMILES: CC#CCc1nc2c(N3C[C@@H](CC)N(C(C)c4ccc(C(F)(F)F)cc4)C[C@@H]3CC)nc(=O)n(C)c2n1C]

A mixture of 8-(but-2-yn-1-yl)-6-((2S,5R)-2,5-diethylpiperazin-1-yl)-3,9-dimethyl-3,9-dihydro-2H-purin-2-one (20 mg, 0.056 mmol), 1-(4-(trifluoromethyl)phenyl)ethan-1-ol (21 mg, 0.112 mmol), (cyanomethyl)trimethylphosphonium iodide (40.8 mg, 0.168 mmol) and DIPEA (36 mg, 0.28 mmol) in CH₃CN (3 mL) was stirred overnight at 100° C. in a sealed tube. The reaction mixture was diluted with EtOAc (20 mL), washed with H₂O, brine, dried over Na₂SO₄, concentrated and purified by Pre-TLC (CH₂Cl₂/MeOH=10:1) to give crude product, which was further purified by Prep-HPLC (Method A) to give the titled compound (70% purity, 3 mg). ¹H NMR (500 MHz, CD₃OD) δ 7.71-7.51 (m, 4H), 6.19-5.06 (m, 2H), 4.03-3.95 (m, 2H), 3.95-3.88 (m, 1H), 3.84-3.76 (m, 3H), 3.76-3.61 (m, 3H), 3.20-2.94 (m, 2H), 2.89-2.51 (m, 1H), 2.43-2.20 (m, 1H), 2.16-1.90 (m, 2H), 1.83-1.72 (m, 3H), 1.53-1.41 (m, 2H), 1.40-1.27 (m, 3H), 1.07-0.86 (m, 3H), 0.74-0.60 (m, 3H) ppm. MS: M/e 529 (M+1)⁺.

### Compound A153: 2-(6-((2S,5R)-2,5-dimethyl-4-(1-(quinoxalin-2-yl)ethyl)piperazin-1-yl)-3,9-dimethyl-2-oxo-3,9-dihydro-2H-purin-8-yl)acetonitrile

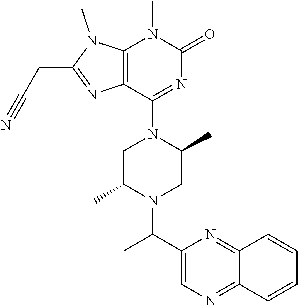 [SMILES: CC(c1cnc2ccccc2n1)N1C[C@H](C)N(c2nc(=O)n(C)c3c2nc(CC#N)n3C)C[C@H]1C]

### Step A: 2-(6-((2S,5R)-2,5-dimethyl-4-(1-(quinoxalin-2-yl)ethyl)piperazin-1-yl)-3,9-dimethyl-2-oxo-3,9-dihydro-2H-purin-8-yl)acetonitrile

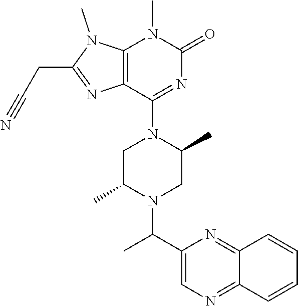 [SMILES: CC(c1cnc2ccccc2n1)N1C[C@H](C)N(c2nc(=O)n(C)c3c2nc(CC#N)n3C)C[C@H]1C]

To a solution of 2-(6-((2S,5R)-2,5-dimethylpiperazin-1-yl)-3,9-dimethyl-2-oxo-3,9-dihydro-2H-purin-8-yl)acetonitrile (50 mg, 0.15 mmol) in CH₃CN (5 mL) and was added 1-(quinoxalin-2-yl)ethan-1-ol (56 mg, 0.32 mmol), (cyanomethyl)trimethylphosphonium iodide (111 mg, 0.45 mmol) and DIPEA (205 mg, 1.57 mmol). The resulting mixture was stirred at 105° C. overnight. The reaction solvent was removed under reduced pressure. The resulting residue was purified by flash column chromatography to give the titled Compound A153 (crude), which was further purified and separated into Compound A153a (1 mg) and Compound A153b (2 mg) by Prep-HPLC (Method A).

Compound A153a (the earlier peak): ¹H NMR (400 MHz, CD₃OD) δ 9.17 (s, 1H), 8.08 (dd, J=12.9, 9.3 Hz, 2H), 7.85 (dd, J=9.5, 5.8 Hz, 2H), 6.18-4.88 (m, 2H), 4.10 (d, J=85.8 Hz, 2H), 3.93 (s, 3H), 3.78 (s, 3H), 3.73-3.33 (m, 3H), 2.84 (s, 1H), 2.11 (d, J=12.2 Hz, 1H), 1.54 (d, J=6.7 Hz, 3H), 1.28 (d, J=5.2 Hz, 3H), 1.12 (d, J=6.4 Hz, 3H) ppm. MS: M/e 472 (M+1)⁺.

Compound A153b (the later peak): ¹H NMR (400 MHz, CD₃OD) δ 9.15 (s, 1H), 8.08 (dd, J=9.3, 6.7 Hz, 2H), 7.83 (p, J=7.8 Hz, 2H), 6.28-4.91 (m, 2H), 4.28-4.08 (m, 2H), 3.93 (s, 3H), 3.78 (s, 3H), 3.71-3.33 (m, 2H), 3.10-2.75 (m, 3H), 1.51 (d, J=6.8 Hz, 3H), 1.41 (d, J=5.6 Hz, 3H), 1.04 (d, J=6.5 Hz, 3H) ppm. MS: M/e 472 (M+1)⁺.

### Compound A156: 2-(9-ethyl-6-((2S,5R)-4-(1-(7-fluoro-2,2-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile

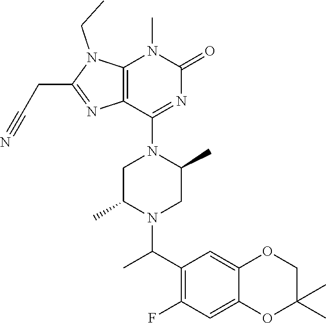 [SMILES: CCn1c(CC#N)nc2c(N3C[C@@H](C)N(C(C)c4cc5c(cc4F)OC(C)(C)CO5)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (33 mg, 0.10 mmol), 1-(7-fluoro-2,2-dimethyl-2,3-dihydrobenzo[b][1,4]dioxin-6-yl)ethan-1-ol (45 mg, 0.20 mmol), (cyanomethyl)trimethylphosphonium iodide (73 mg, 0.30 mmol) and DIPEA (129 mg, 1.00 mmol) in CH₃CN (2 ml) was stirred at 100° C. overnight. The reaction was diluted with EtOAc (10 ml) and washed with brine (10 ml). The organic layer was concentrated under reduced pressure. The resulting residue was purified by flash column chromatography to give the titled Compound A156, which was further separated into Compound A156a (5.99 mg) and Compound A156b (4.55 mg) by Prep-HPLC (Method A).

Compound A156a (the earlier peak): ¹H NMR (400 MHz, DMSO-d₆): δ 6.96 (d, J=6.8 Hz, 1H), 6.72 (d, J=10.9 Hz, 1H), 5.95-5.55 (m, 1H), 4.95-4.60 (m, 1H), 4.40 (s, 2H), 4.26 (q, J=8.0 Hz, 2H), 3.94 (s, 2H), 3.67 (q, J=6.4 Hz, 2H), 3.61 (s, 3H), 3.44 (s, 2H), 2.17 (d, J=11.9 Hz, 1H), 1.34 (t, J=7.0 Hz, 3H), 1.27-1.21 (m, 9H), 1.17 (s, 3H), 0.92 (s, 3H) ppm. MS: M/e 538 (M+1)⁺.

Compound A156b (the later peak): ¹H NMR (400 MHz, DMSO-d₆): δ 6.90 (d, J=7.0 Hz, 1H), 6.75 (d, J=10.7 Hz, 1H), 6.05-5.37 (m, 1H), 5.12-4.54 (m, 1H), 4.39 (s, 2H), 4.25 (s, 2H), 3.95 (s, 2H), 3.83 (q, J=6.4 Hz, 1H), 3.61 (s, 3H), 3.27-3.00 (m, 1H), 2.89-2.64 (m, 3H), 1.37-1.25 (m, 12H), 1.22 (d, J=6.6 Hz, 3H), 0.83 (d, J=6.3 Hz, 3H) ppm. MS: M/e 538 (M+1)⁺.

### Compound A157: 2-(9-ethyl-6-((2S,5R)-4-(1-(7-fluoro-3,3-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile

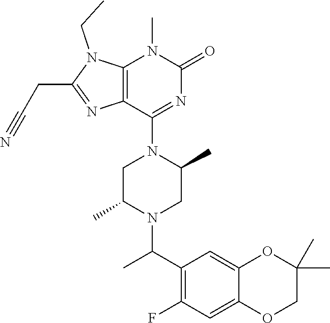 [SMILES: CCn1c(CC#N)nc2c(N3C[C@@H](C)N(C(C)c4cc5c(cc4F)OCC(C)(C)O5)C[C@@H]3C)nc(=O)n(C)c21]

### Step A: 7-fluoro-3,3-dimethyl-2,3-dihydrobenzo[b][1,4]dioxine-6-carbaldehyde

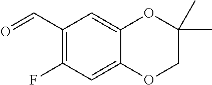

A solution of 7-fluoro-3,3-dimethyl-2,3-dihydrobenzo[b][1,4]dioxine-6-carbonitrile (300 mg, 1.45 mmol) and Dibal-H (1.5 M, 1.93 ml, 2.90 mmol) in THE (10 ml) was stirred at 0° C. under N₂ for 30 min and then RT for 2 hrs. The solution was extracted with EtOAc (15 ml), washed with brine (10 ml×2), dried and evaporated. The residue was purified by flash column chromatography with 0-20% EtOAc in PE to give the titled compound (180 mg, 59%). MS: M/e 211 (M+1)⁺.

### Step B: 1-(7-fluoro-3,3-dimethyl-2,3-dihydrobenzo[b][1,4]dioxin-6-yl)ethan-1-ol

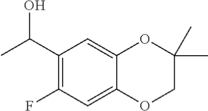 [SMILES: CC(O)c1cc2c(cc1F)OCC(C)(C)O2]

A solution of 7-fluoro-3,3-dimethyl-2,3-dihydrobenzo[b][1,4]dioxine-6-carbaldehyde (180 mg, 0.85 mmol) and MeMgBr (3M, 0.43 ml, 1.29 mmol) in THE (6 ml) was stirred at 0° C. under N₂ for 30 min and then RT for 1 h. The solution was extracted with EtOAc (15 ml), washed with brine (10 ml×2), dried and evaporated. The residue was purified by flash column chromatography with 0-25% EtOAc in PE to give the titled compound (170 mg, 88%). MS: M/e 227 (M+1)⁺.

### Step C: 2-(9-ethyl-6-((2S,5R)-4-(1-(7-fluoro-3,3-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile

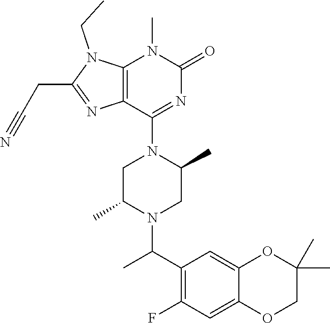 [SMILES: CCn1c(CC#N)nc2c(N3C[C@@H](C)N(C(C)c4cc5c(cc4F)OCC(C)(C)O5)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (146 mg, 0.44 mmol), 1-(7-fluoro-3,3-dimethyl-2,3-dihydrobenzo[b][1,4]dioxin-6-yl)ethan-1-ol (100 mg, 0.44 mmol), (cyanomethyl)trimethylphosphonium iodide (324 mg, 1.33 mmol) and DIPEA (572 mg, 4.43 mmol) in MeCN (4 ml) was stirred at 100° C. overnight. The reaction was diluted with EtOAc (20 ml) and washed with brine (10 ml). The organic layer was concentrated under reduced pressure. The resulting residue was purified by flash column chromatography with 0-5% MeOH in DCM to give the titled Compound A157, which was further separated into Compound A157a (27 mg) and Compound A157b (27 mg) by chiral prep-HPLC. The chiral separation conditions are shown below.

| | |
|---|---|
| Column | CHIRALPAK IH |
| Column Size | 2 cm × 25 cm, 5 um |
| Mobile Phase A | Hex:DCM |
| Mobile Phase B | IPA |
| Flow Rate | 20 mL/min |
| Wave Length | UV 220 nm |
| Temperature | 25° C. |
| Prep-HPLC Equipment | Prep-HPLC-Gilson |

Compound A157a (the earlier peak): ¹H NMR (500 MHz, DMSO-d6) δ 6.97 (d, J=6.9 Hz, 1H), 6.67 (d, J=10.9 Hz, 1H), 6.02-5.45 (m, 1H), 5.12-4.45 (m, 1H), 4.44-4.34 (m, 2H), 4.32-4.18 (m, 2H), 3.93 (s, 2H), 3.83 (q, J=6.6 Hz, 1H), 3.61 (s, 3H), 3.31-3.24 (m, 1H), 2.88-2.66 (m, 3H), 1.37-1.27 (m, 12H), 1.23 (d, J=6.4 Hz, 3H), 0.83 (d, J=5.9 Hz, 3H) ppm. MS: M/e 538 (M+1)⁺.

Compound A157b (the later peak): ¹H NMR (500 MHz, DMSO-d6) δ 7.03 (d, J=6.9 Hz, 1H), 6.64 (d, J=11.0 Hz, 1H), 5.90-5.65 (m, 1H), 4.95-4.60 (m, 1H), 4.46-4.34 (m, 2H), 4.33-4.20 (m, 2H), 3.91 (s, 2H), 3.68 (q, J=6.0 Hz, 1H), 3.61 (s, 3H), 3.50-3.45 (m, 1H), 3.32-3.20 (m, 1H), 2.62-2.52 (m, 1H), 2.18 (d, J=11.7 Hz, 1H), 1.34 (t, J=7.1 Hz, 3H), 1.28 (s, 6H), 1.25 (d, J=6.4 Hz, 3H), 1.18 (s, 3H), 0.94 (s, 3H) ppm. MS: M/e 538 (M+1)⁺.

### Compound A158: 2-(6-((2S,5R)-4-(1-(3,3-dimethyl-2,3-dihydro-[1,4]dioxino[2,3-b]pyridin-6-yl)ethyl)-5-ethyl-2-methylpiperazin-1-yl)-9-ethyl-3-methyl-2-oxo-3,9-dihydro-2H-purin-8-yl)acetonitrile

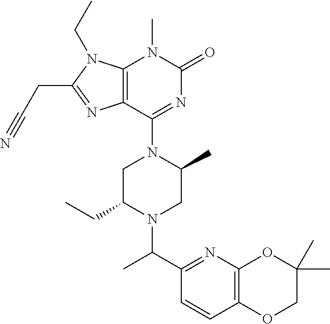 [SMILES: CC[C@@H]1CN(c2nc(=O)n(C)c3c2nc(CC#N)n3CC)[C@@H](C)CN1C(C)c1ccc2c(n1)OC(C)(C)CO2]

### Step A: tert-butyl (2S,5R)-4-(1-(3,3-dimethyl-2,3-dihydro-[1,4]dioxino[2,3-b]pyridin-6-yl)ethyl)-5-ethyl-2-methylpiperazine-1-carboxylate

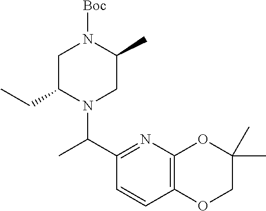 [SMILES: CC[C@@H]1CN(C(=O)OC(C)(C)C)[C@@H](C)CN1C(C)c1ccc2c(n1)OC(C)(C)CO2]

A mixture of tert-butyl (2S,5R)-5-ethyl-2-methylpiperazine-1-carboxylate (228 mg, 1 mmol), 1-(3,3-dimethyl-2,3-dihydro-[1,4]dioxino[2,3-b]pyridin-6-yl)ethan-1-ol (313.5 mg, 1.5 mmol), (cyanomethyl)trimethylphosphonium iodide (729 mg, 3 mmol) and DIPEA (1.29 g, 10 mmol) in CH₃CN (5 mL) was stirred overnight at 100° C. in a sealed tube. The reaction mixture was concentrated to give the residue, which was purified by flash column chromatography to give the titled compound (240 mg, 57%). MS: M/e 420 (M+1)⁺.

### Step B: 6-(1-((2R,5S)-2-ethyl-5-methylpiperazin-1-yl)ethyl)-3,3-dimethyl-2,3-dihydro-[1,4]dioxino[2,3-b]pyridine

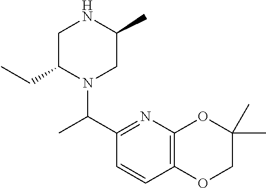 [SMILES: CC[C@@H]1CN[C@@H](C)CN1C(C)c1ccc2c(n1)OC(C)(C)CO2]

To a stirred solution of tert-butyl (2S,5R)-4-(1-(3,3-dimethyl-2,3-dihydro-[1,4]dioxino[2,3-b]pyridin-6-yl)ethyl)-5-ethyl-2-methylpiperazine-1-carboxylate (240 mg, 0.57 mmol) in CH₂Cl₂ (5 mL) was added TFA (2 mL). After the addition, the reaction mixture was stirred for 5 hours. The reaction mixture was concentrated, basified to pH=8˜9 with aq.Na₂CO₃, and extracted with CH₂Cl₂ (10 mL×3). The combined organic layers were washed with brine, dried over Na₂SO₄, concentrated to give the titled compound (180 mg, 98%) as colorless oil. MS: M/e 320 (M+1)⁺.

### Step C: 9-ethyl-6-hydroxy-8-(hydroxymethyl)-3-methyl-3,9-dihydro-2H-purin-2-one

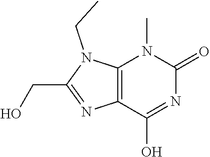 [SMILES: CCn1c(CO)nc2c(O)nc(=O)n(C)c21]

A mixture of 8-((benzyloxy)methyl)-9-ethyl-6-hydroxy-3-methyl-3,9-dihydro-2H-purin-2-one (4.4 g, 14 mmol) in MeOH/AcOH (300 mL/8 mL) was stirred at 70° C. for an hour to become a solution. Then Pd/C (0.5 g) was added, and the mixture was stirred at 40° C. overnight under H₂ (1 atm). The reaction mixture was filtered, and the filtrate was concentrated to give the titled compound (3 g, 95%). MS: M/e 225 (M+1)⁺.

### Step D: 8-(chloromethyl)-9-ethyl-6-hydroxy-3-methyl-3,9-dihydro-2H-purin-2-one

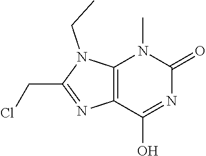 [SMILES: CCn1c(CCl)nc2c(O)nc(=O)n(C)c21]

To a stirred mixture of 9-ethyl-6-hydroxy-8-(hydroxymethyl)-3-methyl-3,9-dihydro-2H-purin-2-one (1 g, 4.46 mmol) in CH₂Cl₂ (20 mL) was added SOCl₂ (3.18 g, 26.78 mmol) at 40° C. overnight. The reaction mixture was filtered, and the cake was collected, dried to give the titled compound (1.15 g, 100%). MS: M/e 243 (M+1)⁺.

### Step E: 2-(9-ethyl-6-hydroxy-3-methyl-2-oxo-3,9-dihydro-2H-purin-8-yl)acetonitrile

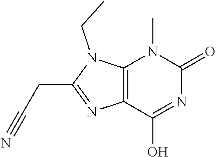 [SMILES: CCn1c(CC#N)nc2c(O)nc(=O)n(C)c21]

To a stirred mixture of 8-(chloromethyl)-9-ethyl-6-hydroxy-3-methyl-3,9-dihydro-2H-purin-2-one (1.15 g, 4.74 mmol) in CH₂Cl₂ (20 mL) was added TMSCN (0.94 g, 9.48 mmol), followed by TBAF (1.0M, 9.48 mL, 9.48 mmol). After the addition, the reaction was stirred overnight. The reaction mixture was filtered, and the cake was collected to give part 1 (338 mg), and the filtrate was concentrated and purified by flash column chromatography to give part 2 (222 mg), then combined to give the titled compound (560 mg, 51%). MS: M/e 234 (M+1)⁺.

### Step F: 2-(6-((2S,5R)-4-(1-(3,3-dimethyl-2,3-dihydro-[1,4]dioxino[2,3-b]pyridin-6-yl)ethyl)-5-ethyl-2-methylpiperazin-1-yl)-9-ethyl-3-methyl-2-oxo-3,9-dihydro-2H-purin-8-yl)acetonitrile

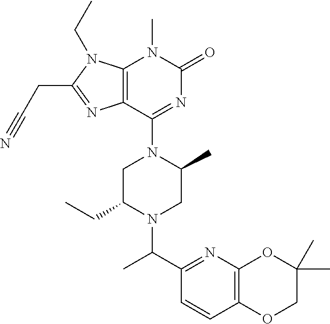 [SMILES: CC[C@@H]1CN(c2nc(=O)n(C)c3c2nc(CC#N)n3CC)[C@@H](C)CN1C(C)c1ccc2c(n1)OC(C)(C)CO2]

To a stirred mixture of 2-(9-ethyl-6-hydroxy-3-methyl-2-oxo-3,9-dihydro-2H-purin-8-yl)acetonitrile (70 mg, 0.3 mmol) in C₂H₄Cl₂ (4 mL) was added DIPEA (77.4 mg, 0.6 mmol), followed by POCl₃ (92.1 mg, 0.6 mmol). After then, the reaction mixture was stirred at 60° C. for 2 hours. The reaction mixture was concentrated to give the residue, which was dissolved in C₂H₄Cl₂ (10 mL) again, DIPEA (77.4 mg, 0.6 mmol) and 6-(1-((2R,5S)-2-ethyl-5-methylpiperazin-1-yl)ethyl)-3,3-dimethyl-2,3-dihydro-[1,4]dioxino[2,3-b]pyridine (95 mg, 0.3 mmol) were added and the mixture was stirred at 100° C. for 3 days. The reaction mixture was washed with H₂O, brine, extracted with CH₂Cl₂ (5 mL×3). The combined organic layers were washed with brine, dried over Na₂SO₄, concentrated. The resulting residue was purified by Prep-HPLC (Method A) to give the titled Compound A158 (17 mg), which was further separated into Compound A158a (7 mg) and Compound A158b (7 mg) by chiral Prep-HPLC. The chiral separation conditions are shown below.

| | |
|---|---|
| Column | CHIRALPAK IF |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A158: ¹H NMR (400 MHz, CD₃OD) δ 7.31 (t, J=7.2 Hz, 1H), 7.12 (dd, J=22.8, 8.0 Hz, 1H), 6.20-4.98 (m, 2H), 4.43-4.31 (m, 2H), 4.22 (s, 0.5H), 4.00-3.91 (m, 2H), 3.76 (s, 3H), 3.73-3.43 (m, 1.5H), 3.30-3.20 (m, 2H), 3.19-2.73 (m, 2H), 2.50-2.16 (m, 1H), 1.56-1.22 (m, 17H), 1.07-0.72 (m, 3H) ppm. MS: M/e 535 (M+1)⁺.

Compound A158a (the earlier peak): ¹H NMR (400 MHz, CD₃OD) δ 7.32 (d, J=8.0 Hz, 1H), 7.09 (d, J=8.0 Hz, 1H), 6.14-5.06 (m, 2H), 4.45-4.26 (m, 2H), 4.21 (s, 0.5H), 3.97 (s, 2H), 3.76 (s, 3H), 3.74-3.67 (m, 1H), 3.52-3.38 (m, 0.5H), 3.30-3.20 (m, 2H), 2.96-2.72 (m, 2H), 2.50-2.38 (m, 1H), 1.61-1.26 (m, 17H), 0.76 (t, J=6.6 Hz, 3H) ppm. MS: M/e 535 (M+1)⁺.

Compound A158b (the later peak): ¹H NMR (400 MHz, CD₃OD) δ 7.30 (d, J=8.0 Hz, 1H), 7.15 (d, J=8.0 Hz, 1H), 6.18-4.99 (m, 2H), 4.43-4.32 (m, 2H), 3.95 (s, 2H), 3.76 (s, 3H), 3.72-3.49 (m, 2H), 3.30-3.20 (m, 2H), 3.18-3.04 (m, 1H), 2.85-2.60 (m, 1H), 2.20 (d, J=11.2 Hz, 1H), 1.55-1.22 (m, 17H), 1.01 (t, J=6.4 Hz, 3H) ppm. MS: M/e 535 (M+1)⁺.

### Compound A159: 2-(6-((2S,5R)-4-(1-(3,3-dimethyl-2,3-dihydro-[1,4]dioxino[2,3-b]pyridin-6-yl)ethyl)-5-ethyl-2-methylpiperazin-1-yl)-3,9-dimethyl-2-oxo-3,9-dihydro-2H-purin-8-yl)acetonitrile

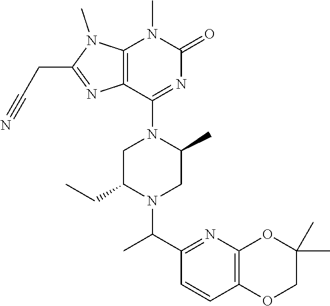 [SMILES: CC[C@@H]1CN(c2nc(=O)n(C)c3c2nc(CC#N)n3C)[C@@H](C)CN1C(C)c1ccc2c(n1)OC(C)(C)CO2]

### Step A: tert-butyl (2R,5S)-4-(8-((benzyloxy)methyl)-3,9-dimethyl-2-oxo-3,9-dihydro-2H-purin-6-yl)-2-ethyl-5-methylpiperazine-1-carboxylate

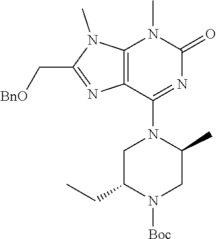 [SMILES: CC[C@@H]1CN(c2nc(=O)n(C)c3c2nc(COCc2ccccc2)n3C)[C@@H](C)CN1C(=O)OC(C)(C)C]

To a stirred mixture of 8-((benzyloxy)methyl)-6-hydroxy-3,9-dimethyl-3,9-dihydro-2H-purin-2-one (1.5 g, 5 mmol) and tert-butyl (2R,5S)-2-ethyl-5-methylpiperazine-1-carboxylate (1.37 g, 6 mmol) in DMF (10 mL) was added BOP (3.3 g, 7.5 mmol), followed by DBU (1.52 g, 10 mmol). After the addition, the reaction mixture was stirred overnight. The reaction mixture was concentrated to give the residue, which was treated with H₂O (20 mL), extracted with EtOAc (30 mL×3). The combined organic layers were washed with brine, dried over Na₂SO₄, concentrated and purified by flash column chromatography to give the titled compound (2.1 g, 82%). MS: M/e 511 (M+1)⁺.

### Step B: tert-butyl (2R,5S)-2-ethyl-4-(8-(hydroxymethyl)-3,9-dimethyl-2-oxo-3,9-dihydro-2H-purin-6-yl)-5-methylpiperazine-1-carboxylate

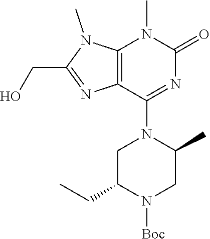 [SMILES: CC[C@@H]1CN(c2nc(=O)n(C)c3c2nc(CO)n3C)[C@@H](C)CN1C(=O)OC(C)(C)C]

To a stirred solution of tert-butyl (2R,5S)-4-(8-((benzyloxy)methyl)-3,9-dimethyl-2-oxo-3,9-dihydro-2H-purin-6-yl)-2-ethyl-5-methylpiperazine-1-carboxylate (2.1 g, 4.11 mmol) in MeOH/AcOH (150 mL/5 mL) was added Pd/C (300 mg). After the addition, the reaction mixture was stirred at 35° C. over a weekend under H₂ (1 atm). The reaction mixture was filtered, and the filtrate was concentrated to give the titled compound (1.7 g, 98%). MS: M/e 421 (M+1)⁺.

### Step C: tert-butyl (2R,5S)-4-(8-(cyanomethyl)-3,9-dimethyl-2-oxo-3,9-dihydro-2H-purin-6-yl)-2-ethyl-5-methylpiperazine-1-carboxylate

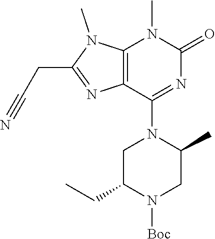 [SMILES: CC[C@@H]1CN(c2nc(=O)n(C)c3c2nc(CC#N)n3C)[C@@H](C)CN1C(=O)OC(C)(C)C]

To a stirred solution of tert-butyl (2R,5S)-2-ethyl-4-(8-(hydroxymethyl)-3,9-dimethyl-2-oxo-3,9-dihydro-2H-purin-6-yl)-5-methylpiperazine-1-carboxylate (1.7 g, 4 mmol) in CH₂Cl₂ (15 mL) was added SOCl₂ (0.96 g, 8 mmol). After the addition, the reaction mixture was stirred for 30 min. The reaction mixture was concentrated to give the residue, which was dissolved in CH₂Cl₂ (20 mL) again. TMSCN (0.8 g, 8 mmol) was added followed by TBAF (1.0 M, 8 mL, 8 mmol). After then, the reaction was stirred for 4 hours. The reaction mixture was washed with H₂O, brine, dried over Na₂SO₄, concentrated and purified by flash column chromatography to give the titled compound (400 mg, 23%). MS: M/e 430 (M+1)⁺.

### Step D: 2-(6-((2S,5R)-5-ethyl-2-methylpiperazin-1-yl)-3,9-dimethyl-2-oxo-3,9-dihydro-2H-purin-8-yl)acetonitrile

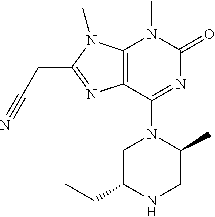 [SMILES: CC[C@@H]1CN(c2nc(=O)n(C)c3c2nc(CC#N)n3C)[C@@H](C)CN1]

To a stirred solution of tert-butyl (2R,5S)-4-(8-(cyanomethyl)-3,9-dimethyl-2-oxo-3,9-dihydro-2H-purin-6-yl)-2-ethyl-5-methylpiperazine-1-carboxylate (400 mg, 0.93 mmol) in CH₂Cl₂ (5 mL) was added EtOAc/HCl(g) (1.0 M, 10 mL). After then, the reaction mixture was stirred for 4 hours. The reaction mixture was concentrated to give the residue, which was treated with H₂O (2 mL), then Na₂CO₃ (0.5 g) was added, then washed with CH₃CN (15 mL), stirred for 10 min and filtered. The filtrate was dried over Na₂SO₄, concentrated to give the titled compound (300 mg, 98%). MS: M/e 330 (M+1)⁺.

### Step E: 2-(6-((2S,5R)-4-(1-(3,3-dimethyl-2,3-dihydro-[1,4]dioxino[2,3-b]pyridin-6-yl)ethyl)-5-ethyl-2-methylpiperazin-1-yl)-3,9-dimethyl-2-oxo-3,9-dihydro-2H-purin-8-yl)acetonitrile

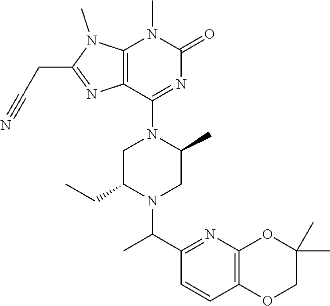 [SMILES: CC[C@@H]1CN(c2nc(=O)n(C)c3c2nc(CC#N)n3C)[C@@H](C)CN1C(C)c1ccc2c(n1)OC(C)(C)CO2]

A mixture of 2-(6-((2S,5R)-5-ethyl-2-methylpiperazin-1-yl)-3,9-dimethyl-2-oxo-3,9-dihydro-2H-purin-8-yl)acetonitrile (100 mg, 0.304 mmol), 1-(3,3-dimethyl-2,3-dihydro-[1,4]dioxino[2,3-b]pyridin-6-yl)ethan-1-ol (190 mg, 0.9 mmol), (cyanomethyl)trimethylphosphonium iodide (221 mg, 0.9 mmol) and DIPEA (196 mg, 1.52 mmol) in CH₃CN (4 mL) was stirred overnight at 100° C. in a sealed tube. The reaction mixture was diluted with EtOAc (20 mL), washed with H₂O, brine, dried over Na₂SO₄, concentrated to dryness. The resulting residue was purified by Prep-HPLC (Method A) to give the titled Compound A159 (28 mg), which was further separated into Compound A159a (5 mg) and Compound A159b (6 mg) by chiral Prep-HPLC. The chiral separation conditions are shown below.

| | |
|---|---|
| Column | CHIRALPAK ID |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A159: ¹H NMR (400 MHz, CD₃OD) δ 7.32 (t, J=6.8 Hz, 1H), 7.11 (t, J=8.4 Hz, 1H), 6.24-5.00 (m, 2H), 4.22 (s, 1H), 3.97 (d, J=2.4 Hz, 2H), 3.93 (s, 3H), 3.88-3.82 (m, 1H), 3.79 (s, 3H), 3.75-3.50 (m, 1H), 3.27-2.36 (m, 4H), 1.64-1.23 (m, 14H), 1.11-0.75 (m, 3H) ppm. MS: M/e 521 (M+1)⁺.

Compound A159a (the earlier peak): ¹H NMR (400 MHz, CD₃OD) δ 7.32 (d, J=8.0 Hz, 1H), 7.09 (d, J=8.0 Hz, 1H), 6.15-5.04 (m, 2H), 3.96 (s, 2H), 3.92 (s, 3H), 3.78 (s, 3H), 3.76-3.63 (m, 1H), 3.50-3.38 (m, 1H), 3.30-3.20 (m, 1H), 3.14-2.71 (m, 3H), 2.48-2.36 (m, 1H), 1.57-1.26 (m, 14H), 0.74 (t, J=6.8 Hz, 3H) ppm. MS: M/e 521 (M+1)⁺.

Compound A159b (the earlier peak): ¹H NMR (400 MHz, CD₃OD) δ 7.30 (d, J=8.0 Hz, 1H), 7.15 (d, J=8.0 Hz, 1H), 6.17-4.95 (m, 2H), 3.95 (s, 2H), 3.93 (s, 3H), 3.78 (s, 3H), 3.71-3.43 (m, 2H), 3.30-3.20 (m, 2H), 3.15-3.03 (m, 1H), 2.79-2.60 (m, 1H), 2.19 (d, J=11.2 Hz, 1H), 1.52-1.42 (m, 2H), 1.40 (s, 6H), 1.35-1.22 (m, 6H), 1.00 (t, J=6.4 Hz, 3H) ppm. MS: M/e 521 (M+1)⁺.

### Compound A161: 2-(9-ethyl-6-((2S,5R)-4-(1-(7-fluoro-3,3-dimethyl-2,3-dihydro-[1,4]dioxino[2,3-b]pyridin-6-yl)ethyl)-2,5-dimethylpiperazin-1-yl)-3-methyl-2-oxo-3,9-dihydro-2H-purin-8-yl)acetonitrile

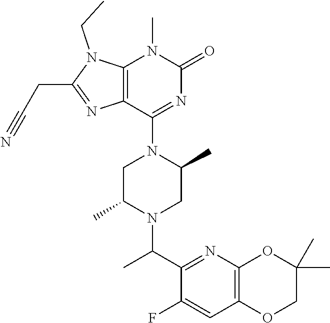 [SMILES: CCn1c(CC#N)nc2c(N3C[C@@H](C)N(C(C)c4nc5c(cc4F)OCC(C)(C)O5)C[C@@H]3C)nc(=O)n(C)c21]

### Step A: 2-chloro-5-fluoro-6-(hydroxymethyl)pyridin-3-ol

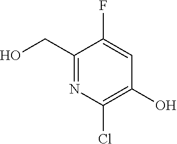 [SMILES: OCc1nc(Cl)c(O)cc1F]

A mixture of 2-chloro-5-fluoropyridin-3-ol (2 g, 13.5 mmol) in water (10 mL) was added NaHCO₃ (1.7 g, 20.3 mmol) at room temperature and the resulting mixture was stirred at room temperature for 15 mins. Then was added HCHO (5.5 mL in water, 37%) at room temperature and the resulting mixture was stirred at 90° C. under N₂ for 5 hours. Then the reaction mixture was treated with HCl (1M) to pH˜4, extracted with EtOAc dried over Na₂SO₄ and concentrated to dryness. The resulting residue was purified by flash column chromatography to give the titled compound (1.8 g, 75%). MS: M/e 178 (M+1)⁺.

### Step B: 1-((2-chloro-5-fluoro-6-(hydroxymethyl)pyridin-3-yl)oxy)propan-2-one

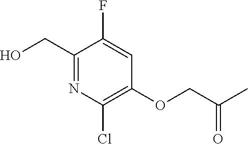

To a solution of 2-chloro-5-fluoro-6-(hydroxymethyl)pyridin-3-ol (1.8 g, 10.1 mmol) in CH₃CN (20 mL) was added K₂CO₃ (2.8 g, 20.3 mmol) and 1-chloropropan-2-one at (1.58 g, 16.9 mmol) room temperature and the resulting mixture was stirred at 60° C. for 15 hours. The reaction mixture was concentrated to dryness. The resulting residue was purified by flash column chromatography to give the titled compound (1.5 g, 63%). MS: M/e 234 (M+1)⁺.

### Step C: 6-chloro-3-fluoro-5-(2-oxopropoxy)picolinaldehyde

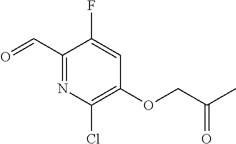

To a solution of 1-((2-chloro-5-fluoro-6-(hydroxymethyl)pyridin-3-yl)oxy)propan-2-one (1.5 g, 6.4 mmol) in DCM (50 mL) was added Dess Martin (4.1 g, 9.6 mmol) at room temperature and the resulting mixture was stirred at room temperature for 1.5 hours. The reaction mixture was quenched with saturated NaHCO₃ aq., extracted with EtOAc combined, washed brine, dried and concentrated to dryness. The resulting residue was purified by flash column chromatography to give the titled compound (800 mg, 53%). MS: M/e 232 (M+1)⁺.

### Step D: 1-((2-chloro-5-fluoro-6-(1-hydroxyethyl)pyridin-3-yl)oxy)-2-methylpropan-2-ol

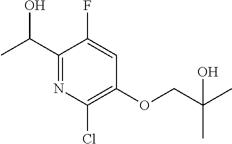 [SMILES: CC(O)c1nc(Cl)c(OCC(C)(C)O)cc1F]

To a solution of 6-chloro-3-fluoro-5-(2-oxopropoxy)picolinaldehyde (800 mg, 3.5 mmol) in THE (50 mL) was added CH₃MgBr (3M, 5.7 mL, 17.3 mmol) at room temperature and the resulting mixture was stirred at room temperature for 1 hour. The reaction mixture was quenched with saturated NH₄Cl aq. (50 mL), extracted with EtOAc washed brine, dried and concentrated to give the titled compound (820 mg, 89%). MS: M/e 264 (M+1)⁺.

### Step E: 1-(7-fluoro-3,3-dimethyl-2,3-dihydro-[1,4]dioxino[2,3-b]pyridin-6-yl)ethan-1-ol

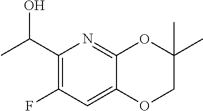 [SMILES: CC(O)c1nc2c(cc1F)OCC(C)(C)O2]

A mixture of 1-((2-chloro-5-fluoro-6-(1-hydroxyethyl)pyridin-3-yl)oxy)-2-methylpropan-2-ol (800 mg, 3 mmol), BINAP (379 mg, 0.6 mmol), Cs₂CO₃ (1.9 g, 6 mmol) and Pd(AcO)₂ (68 mg, 0.3 mmol) in toluene (50 mL) was stirred at 100° C. under N₂ for 15 hours. The reaction mixture was concentrated to dryness. The resulting residue was purified by flash column chromatography to give the titled compound (370 mg, 53%). MS: M/e 228 (M+1)⁺.

### Step F: 2-(9-ethyl-6-((2S,5R)-4-(1-(7-fluoro-3,3-dimethyl-2,3-dihydro-[1,4]dioxino[2,3-b]pyridin-6-yl)ethyl)-2,5-dimethylpiperazin-1-yl)-3-methyl-2-oxo-3,9-dihydro-2H-purin-8-yl)acetonitrile

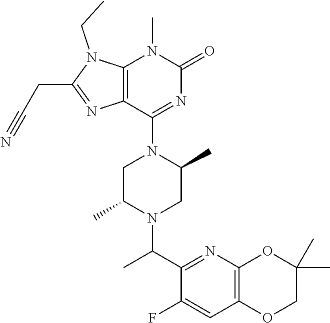 [SMILES: CCn1c(CC#N)nc2c(N3C[C@@H](C)N(C(C)c4nc5c(cc4F)OCC(C)(C)O5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (300 mg, 0.9 mmol) in CH₃CN (8 mL) and was added 1-(7-fluoro-3,3-dimethyl-2,3-dihydro-[1,4]dioxino[2,3-b]pyridin-6-yl)ethan-1-ol (310 mg, 1.35 mmol), (cyanomethyl)trimethylphosphonium iodide (875 mg, 3.6 mmol) and DIPEA (1.16 g, 9 mmol). The resulting mixture was stirred at 105° C. overnight. The reaction solvent was removed under reduced pressure and purified by flash column chromatography (DCM/MeOH) to give the titled Compound A161, which was further separated into Compound A161a (113 mg) and Compound A161b (115 mg) by chiral Prep-HPLC. The chiral separation conditions are shown below.

| | |
|---|---|
| Column | CHIRALPAK IA |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A161: ¹H NMR (400 MHz, DMSO-d6) δ 7.22-7.14 (m, 1H), 6.15-4.94 (m, 2H), 4.36 (d, J=7.4 Hz, 2H), 4.23 (s, 1H), 4.07 (s, 1H), 3.99 (s, 2H), 3.76 (s, 3H), 3.73-3.36 (m, 2H), 3.10-2.67 (m, 2H), 2.49-2.10 (m, 1H), 1.54-1.22 (m, 15H), 1.10-0.90 (m, 3H) ppm. MS: M/e 539 (M+1)⁺.

Compound A161a (the earlier peak): ¹H NMR (400 MHz, CD₃OD) δ 7.17 (d, J=9.6 Hz, 1H), 6.17-4.92 (m, 2H), 4.36 (d, J=7.0 Hz, 2H), 4.03 (d, J=6.6 Hz, 1H), 3.98 (s, 2H), 3.76 (s, 3H), 3.60-3.34 (m, 3H), 3.10-2.62 (m, 3H), 1.54-1.27 (m, 15H), 0.97 (d, J=6.2 Hz, 3H) ppm. MS: M/e 539 (M+1)⁺.

Compound A161b (the later peak): ¹H NMR (400 MHz, CD₃OD) δ 7.14 (d, J=9.4 Hz, 1H), 6.11-4.88 (m, 2H), 4.36 (d, J=7.3 Hz, 2H), 3.98 (s, 2H), 3.89 (s, 1H), 3.75 (s, 3H), 3.73-3.35 (m, 4H), 2.80 (s, 1H), 2.20 (d, J=11.4 Hz, 1H), 1.46 (t, J=7.0 Hz, 3H), 1.38 (d, J=7.9 Hz, 9H), 1.24 (s, 3H), 1.00 (d, J=5.6 Hz, 3H) ppm. MS: M/e 539 (M+1)⁺.

### Compound A162: 2-(6-((2S,5R)-4-(1-(7-fluoro-3,3-dimethyl-2,3-dihydro-[1,4]dioxino[2,3-b]pyridin-6-yl)ethyl)-2,5-dimethylpiperazin-1-yl)-3,9-dimethyl-2-oxo-3,9-dihydro-2H-purin-8-yl)acetonitrile

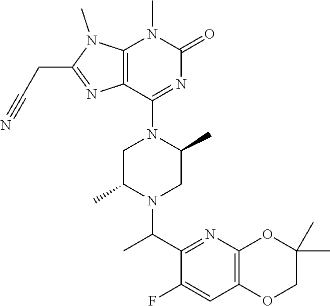 [SMILES: CC(c1nc2c(cc1F)OCC(C)(C)O2)N1C[C@H](C)N(c2nc(=O)n(C)c3c2nc(CC#N)n3C)C[C@H]1C]

To a solution of 2-(6-((2S,5R)-2,5-dimethylpiperazin-1-yl)-3,9-dimethyl-2-oxo-3,9-dihydro-2H-purin-8-yl)acetonitrile (50 mg, 0.16 mmol) in CH₃CN (8 mL) and was added 1-(7-fluoro-3,3-dimethyl-2,3-dihydro-[1,4]dioxino[2,3-b]pyridin-6-yl)ethan-1-ol (54 mg, 0.24 mmol), (cyanomethyl)trimethylphosphonium iodide (115 mg, 0.48 mmol) and DIPEA (204 mg, 9 mmol). The resulting mixture was stirred at 105° C. overnight. The reaction solvent was removed under reduced pressure and purified by flash column chromatography (DCM/MeOH) to give the titled compound (16 mg). ¹H NMR (400 MHz, CD₃OD) δ 7.21-7.11 (m, 1H), 6.01-4.91 (m, 2H), 4.21 (s, 1H), 4.04 (s, 1H), 3.98 (s, 2H), 3.93 (s, 3H), 3.78 (s, 3H), 3.70-3.36 (m, 2H), 3.05-2.09 (m, 3H), 1.43-1.22 (m, 12H), 1.04-0.85 (m, 3H) ppm. MS: M/e 525 (M+1)⁺.

### Compound A163: 2-(9-ethyl-6-((2S,5R)-4-(1-(8-fluoro-3,3-dimethyl-2,3-dihydro-[1,4]dioxino[2,3-b]pyridin-6-yl)ethyl)-2,5-dimethylpiperazin-1-yl)-3-methyl-2-oxo-3,9-dihydro-2H-purin-8-yl)acetonitrile

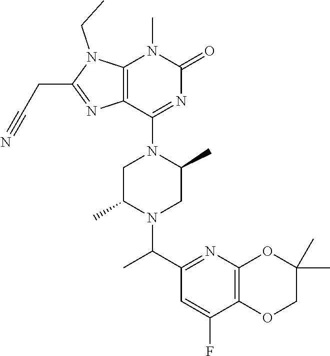 [SMILES: CCn1c(CC#N)nc2c(N3C[C@@H](C)N(C(C)c4cc(F)c5c(n4)OC(C)(C)CO5)C[C@@H]3C)nc(=O)n(C)c21]

### Step A: 2-chloro-4-fluoro-6-(hydroxymethyl)pyridin-3-ol

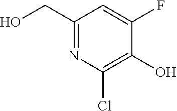 [SMILES: OCc1cc(F)c(O)c(Cl)n1]

A mixture of 2-chloro-4-fluoropyridin-3-ol (0.6 g, 4.05 mmol) in water (3 mL) was added NaHCO₃ (0.51 g, 6.08 mmol) at room temperature and the resulting mixture was stirred at room temperature for 15 mins. Then was added HCHO (8.2 mL, in water, 37%) at room temperature and the resulting mixture was stirred at 110° C. under N₂ for 3 days. Then the reaction mixture was treated with HCl (1M) to pH˜4, extracted with EtOAc dried over Na₂SO₄ and concentrated to dryness. The resulting residue was purified by flash column chromatography to give the titled compound (0.42 g, 58%). MS: M/e 178 (M+1)⁺.

### Step B: 1-((2-chloro-4-fluoro-6-(hydroxymethyl)pyridin-3-yl)oxy)propan-2-one

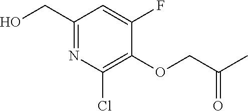

To a solution of 2-chloro-4-fluoro-6-(hydroxymethyl)pyridin-3-ol (0.7 g, 3.9 mmol) in CH₃CN (20 mL) was added K₂CO₃ (1.1 g, 7.9 mmol) and 1-chloropropan-2-one at (0.55 g, 5.9 mmol) room temperature and the resulting mixture was stirred at 60° C. for 15 hours. The reaction mixture was concentrated to dryness. The resulting residue was purified by flash column chromatography to give the titled compound (0.8 g, 86%). MS: M/e 234 (M+1)⁺.

### Step C: 6-chloro-4-fluoro-5-(2-oxopropoxy)picolinaldehyde

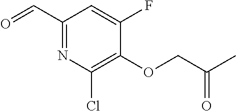

To a solution of 1-((2-chloro-4-fluoro-6-(hydroxymethyl)pyridin-3-yl)oxy)propan-2-one (0.8 g, 3.4 mmol) in DCM (50 mL) was added Dess-Martin periodinane (2.2 g, 51 mmol) at room temperature and the resulting mixture was stirred at room temperature for 1.5 hours. The reaction mixture was quenched with saturated NaHCO₃ aq., extracted with EtOAc combined, washed brine, dried and concentrated to dryness. The resulting residue was purified by flash column chromatography to give the titled compound (320 mg, 40%). MS: M/e 232 (M+1)⁺.

### Step D: 1-((2-chloro-4-fluoro-6-(1-hydroxyethyl)pyridin-3-yl)oxy)-2-methylpropan-2-ol

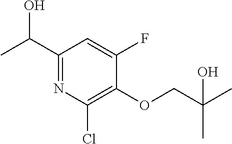 [SMILES: CC(O)c1cc(F)c(OCC(C)(C)O)c(Cl)n1]

To a solution of 6-chloro-4-fluoro-5-(2-oxopropoxy)picolinaldehyde (320 mg, 1.38 mmol) in THE (40 mL) was added CH₃MgBr (3M, 2.3 mL, 6.9 mmol) at room temperature and the resulting mixture was stirred at room temperature for 1 hour. The reaction mixture was quenched with saturated NH₄Cl aq. (50 mL), extracted with EtOAc washed brine, dried and concentrated to give the titled compound (350 mg, 96%). MS: M/e 264 (M+1)⁺.

### Step E: 1-(8-fluoro-3,3-dimethyl-2,3-dihydro-[1,4]dioxino[2,3-b]pyridin-6-yl)ethan-1-ol

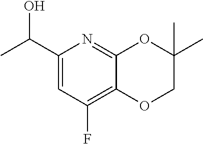 [SMILES: CC(O)c1cc(F)c2c(n1)OC(C)(C)CO2]

A mixture of 1-((2-chloro-4-fluoro-6-(1-hydroxyethyl)pyridin-3-yl)oxy)-2-methylpropan-2-ol (350 mg, 1.3 mmol), BINAP (165 mg, 0.25 mmol), Cs₂CO₃ (0.86 g, 2.6 mmol) and Pd(OAc)₂ (30 mg, 0.13 mmol) in toluene (10 mL) was stirred at 100° C. under N₂ for 15 hours. The reaction mixture was concentrated to dryness. The resulting residue was purified by flash column chromatography to give the titled compound (170 mg, 56%). MS: M/e 228 (M+1)⁺.

### Step F: 2-(9-ethyl-6-((2S,5R)-4-(1-(8-fluoro-3,3-dimethyl-2,3-dihydro-[1,4]dioxino[2,3-b]pyridin-6-yl)ethyl)-2,5-dimethylpiperazin-1-yl)-3-methyl-2-oxo-3,9-dihydro-2H-purin-8-yl)acetonitrile

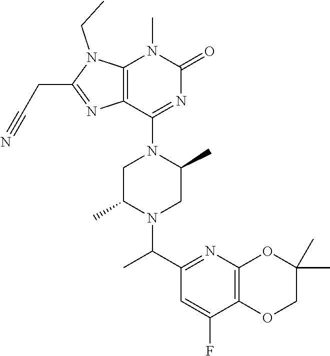 [SMILES: CCn1c(CC#N)nc2c(N3C[C@@H](C)N(C(C)c4cc(F)c5c(n4)OC(C)(C)CO5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (120 mg, 0.36 mmol) in CH₃CN (8 mL) and was added 1-(8-fluoro-3,3-dimethyl-2,3-dihydro-[1,4]dioxino[2,3-b]pyridin-6-yl)ethan-1-ol (124 mg, 0.54 mmol), (cyanomethyl)trimethylphosphonium iodide (353 mg, 1.4 mmol) and DIPEA (464 mg, 3.6 mmol). The resulting mixture was stirred at 105° C. overnight. The reaction solvent was removed under reduced pressure and purified by by flash column chromatography (DCM/MeOH) to give the titled Compound A163, which was further separated into Compound A163a (44 mg) and Compound A163b (42 mg) by chiral Prep-HPLC. The chiral separation conditions are shown below.

| | |
|---|---|
| Column | CHIRALPAK IG |
| Column Size | 2 cm × 25 cm, 5 um |
| Mobile Phase A | MtBE |
| Mobile Phase B | EtOH |
| Flow Rate | 20 mL/min |
| Wave Length | UV 220 nm |
| Temperature | 25° C. |
| Prep-HPLC Equipment | Prep-HPLC-Gilson |

Compound A163: ¹H NMR (400 MHz, CD₃OD) δ 7.05 (t, J=9.5 Hz, 1H), 6.12-4.99 (m, 2H), 4.37 (d, J=6.9 Hz, 2H), 4.22 (s, 1H), 4.01 (s, 2H), 3.76 (s, 3H), 3.69-3.33 (m, 3H), 3.07-2.10 (m, 3H), 1.51-1.39 (m, 10H), 1.37-1.23 (m, 5H), 1.07-0.89 (m, 3H) ppm. MS: M/e 539 (M+1)⁺.

Compound A163a (the earlier peak): ¹H NMR (400 MHz, CD₃OD) δ 7.04 (d, J=10.1 Hz, 1H), 6.28-4.87 (m, 2H), 4.52-4.15 (m, 3H), 4.01 (s, 2H), 3.76 (s, 3H), 3.69-3.32 (m, 3H), 2.89 (s, 2H), 2.78 (d, J=10.9 Hz, 1H), 1.54-1.37 (m, 12H), 1.26 (d, J=6.1 Hz, 3H), 0.94 (d, J=5.9 Hz, 3H) ppm. MS: M/e 539 (M+1)⁺.

Compound A163b (the later peak): ¹H NMR (400 MHz, CD₃OD) δ 7.06 (d, J=10.6 Hz, 1H), 6.14-4.91 (m, 2H), 4.58-4.09 (m, 3H), 4.01 (s, 2H), 3.76 (s, 3H), 3.74-3.64 (m, 1H), 3.46 (d, J=52.3 Hz, 3H), 2.72 (s, 1H), 2.17 (d, J=11.9 Hz, 1H), 1.54-1.25 (m, 15H), 1.01 (d, J=4.5 Hz, 3H) ppm. MS: M/e 539 (M+1)⁺.

### Compound A164: 2-(6-((2S,5R)-4-(1-(8-fluoro-3,3-dimethyl-2,3-dihydro-[1,4]dioxino[2,3-b]pyridin-6-yl)ethyl)-2,5-dimethylpiperazin-1-yl)-3,9-dimethyl-2-oxo-3,9-dihydro-2H-purin-8-yl)acetonitrile

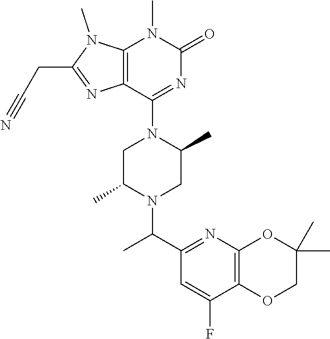 [SMILES: CC(c1cc(F)c2c(n1)OC(C)(C)CO2)N1C[C@H](C)N(c2nc(=O)n(C)c3c2nc(CC#N)n3C)C[C@H]1C]

To a solution of 2-(6-((2S,5R)-2,5-dimethylpiperazin-1-yl)-3,9-dimethyl-2-oxo-3,9-dihydro-2H-purin-8-yl)acetonitrile (50 mg, 0.16 mmol) in CH₃CN (8 mL) and was added 1-(8-fluoro-3,3-dimethyl-2,3-dihydro-[1,4]dioxino[2,3-b]pyridin-6-yl)ethan-1-ol (54 mg, 0.24 mmol), (cyanomethyl)trimethylphosphonium iodide (115 mg, 0.48 mmol) and DIPEA (204 mg, 9 mmol). The resulting mixture was stirred at 105° C. overnight. The reaction solvent was removed under reduced pressure and purified by flash column chromatography (DCM/MeOH) to give the titled compound (12 mg). ¹H NMR (400 MHz, CD₃OD) δ 7.05 (t, J=9.1 Hz, 1H), 6.18-4.95 (m, 2H), 4.21 (s, 1H), 4.01 (s, 2H), 3.93 (s, 3H), 3.79 (s, 3H), 3.73-3.33 (m, 3H), 3.10-2.03 (m, 3H), 1.42 (s, 6H), 1.36-1.24 (m, 6H), 1.05-0.90 (m, 3H) ppm. MS: M/e 525 (M+1)⁺.

### Compound A165: 2-(9-(2,2-difluoroethyl)-6-((2S,5R)-4-(1-(3,3-dimethyl-2,3-dihydro-[1,4]dioxino[2,3-b]pyridin-6-yl)ethyl)-2,5-dimethylpiperazin-1-yl)-3-methyl-2-oxo-3,9-dihydro-2H-purin-8-yl)acetonitrile

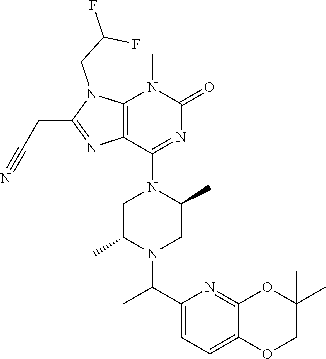 [SMILES: CC(c1ccc2c(n1)OC(C)(C)CO2)N1C[C@H](C)N(c2nc(=O)n(C)c3c2nc(CC#N)n3CC(F)F)C[C@H]1C]

### Step A: tert-butyl (2R,5S)-4-(8-((benzyloxy)methyl)-9-(2,2-difluoroethyl)-3-methyl-2-oxo-3,9-dihydro-2H-purin-6-yl)-2,5-dimethylpiperazine-1-carboxylate

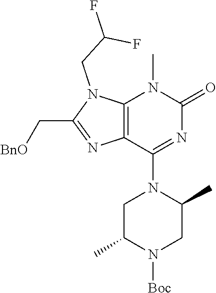 [SMILES: C[C@@H]1CN(c2nc(=O)n(C)c3c2nc(COCc2ccccc2)n3CC(F)F)[C@@H](C)CN1C(=O)OC(C)(C)C]

To a stirred mixture of 8-((benzyloxy)methyl)-9-(2,2-difluoroethyl)-6-hydroxy-3-methyl-3,9-dihydro-2H-purin-2-one (1.5 g, 4.28 mmol) and tert-butyl (2R,5S)-2,5-dimethylpiperazine-1-carboxylate (1.83 g, 8.57 mmol) in CH₃CN (15 mL) was added BOP (4.60 g, 8.57 mmol), followed by DBU (1.3 g, 8.57 mmol). After the addition, the reaction mixture was concentrated to give the residue, which was dissolved in EtOAc (50 mL), washed with H₂O (20 mL), brine, dried over Na₂SO₄, concentrated and purified by flash column chromatography to give the titled compound (1.8 g, 77%). MS: M/e 547 (M+1)⁺.

### Step B: tert-butyl (2R,5S)-4-(9-(2,2-difluoroethyl)-8-(hydroxymethyl)-3-methyl-2-oxo-3,9-dihydro-2H-purin-6-yl)-2,5-dimethylpiperazine-1-carboxylate

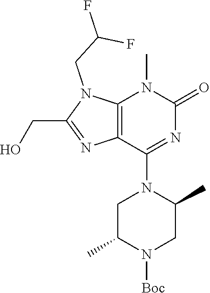 [SMILES: C[C@@H]1CN(c2nc(=O)n(C)c3c2nc(CO)n3CC(F)F)[C@@H](C)CN1C(=O)OC(C)(C)C]

To a stirred solution of tert-butyl (2R,5S)-4-(8-((benzyloxy)methyl)-9-(2,2-difluoroethyl)-3-methyl-2-oxo-3,9-dihydro-2H-purin-6-yl)-2,5-dimethylpiperazine-1-carboxylate (1.8 g, 3.3 mmol) in MeOH/AcOH (80 mL/2 mL) was added Pd/C (200 mg). After the addition, the reaction mixture was stirred over a weekend under H₂ (4 atm). The reaction mixture was filtered, and filtrate was concentrated to give the titled compound (1.22 g, 81%). MS: M/e 457 (M+1)⁺.

### Step C: tert-butyl (2R,5S)-4-(8-(cyanomethyl)-9-(2,2-difluoroethyl)-3-methyl-2-oxo-3,9-dihydro-2H-purin-6-yl)-2,5-dimethylpiperazine-1-carboxylate

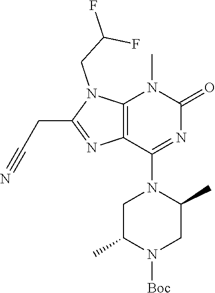 [SMILES: C[C@@H]1CN(c2nc(=O)n(C)c3c2nc(CC#N)n3CC(F)F)[C@@H](C)CN1C(=O)OC(C)(C)C]

To a stirred solution of tert-butyl (2R,5S)-4-(9-(2,2-difluoroethyl)-8-(hydroxymethyl)-3-methyl-2-oxo-3,9-dihydro-2H-purin-6-yl)-2,5-dimethylpiperazine-1-carboxylate (1.22 g, 2.68 mmol) in CH₂Cl₂ (50 mL) was added SOCl₂ (319 mg, 2.68 mmol). After stirred for 10 min, the reaction mixture was quenched with aq.NaHCO₃, washed with brine, dried over Na₂SO₄, concentrated to give the intermediate, dissolved in CH₂Cl₂ (10 mL), TBAF (1.0 M, 3.2 mL, 3.2 mmol) was added followed by TMSCN (0.32 g, 3.2 mmol). After the addition, the reaction mixture was stirred for 3 hours. The reaction mixture was concentrated to give the residue, treated with H₂O and extracted with EtOAc (15 mL×3). The combined organic layers were washed with brine, dried over Na₂SO₄, concentrated and purified by flash column chromatography to give the titled compound (900 mg, 72%). MS: M/e 466 (M+1)⁺.

### Step D: 2-(9-(2,2-difluoroethyl)-6-((2S,5R)-2,5-dimethylpiperazin-1-yl)-3-methyl-2-oxo-3,9-dihydro-2H-purin-8-yl)acetonitrile

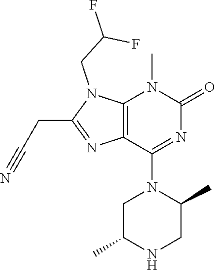 [SMILES: C[C@@H]1CN(c2nc(=O)n(C)c3c2nc(CC#N)n3CC(F)F)[C@@H](C)CN1]

To a stirred solution of tert-butyl (2R,5S)-4-(8-(cyanomethyl)-9-(2,2-difluoroethyl)-3-methyl-2-oxo-3,9-dihydro-2H-purin-6-yl)-2,5-dimethylpiperazine-1-carboxylate (900 mg, 1.93 mmol) in EtOAc (10 mL) was added dioxane/HCl (g) (4.0 M, 4 mL). After the addition, the reaction mixture was stirred for 4 hours. The reaction mixture was concentrated to give the residue, which was treated with CH₃CN/aq.NaHCO₃ (20 mL/3 mL), stirred for 10 min and filtered. The filtrate was concentrated, dried to give the titled compound (560 mg, 79%), which was directly used to the next step. MS: M/e 366 (M+1)⁺.

### Step E: 2-(9-(2,2-difluoroethyl)-6-((2S,5R)-4-(1-(3,3-dimethyl-2,3-dihydro-[1,4]dioxino[2,3-b]pyridin-6-yl)ethyl)-2,5-dimethylpiperazin-1-yl)-3-methyl-2-oxo-3,9-dihydro-2H-purin-8-yl)acetonitrile

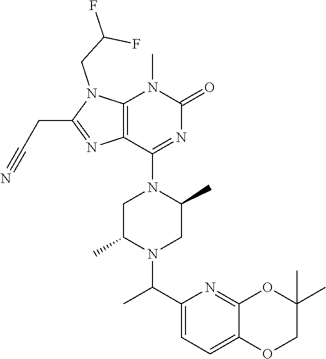 [SMILES: CC(c1ccc2c(n1)OC(C)(C)CO2)N1C[C@H](C)N(c2nc(=O)n(C)c3c2nc(CC#N)n3CC(F)F)C[C@H]1C]

To a solution of 2-(9-(2,2-difluoroethyl)-6-((2S,5R)-2,5-dimethylpiperazin-1-yl)-3-methyl-2-oxo-3,9-dihydro-2H-purin-8-yl)acetonitrile (50 mg, 0.14 mmol) in CH₃CN (3 mL) and was added 1-(3,3-dimethyl-2,3-dihydro-[1,4]dioxino[2,3-b]pyridin-6-yl)ethan-1-ol (29 mg, 0.14 mmol), (cyanomethyl)trimethylphosphonium iodide (100 mg, 0.41 mmol) and DIPEA (88 mg, 0.68 mmol). The resulting mixture was stirred at 105° C. overnight. The reaction solvent was removed under reduced pressure to dryness. The resulting residue was purified by Prep-HPLC (Method A) to give the titled compound (15 mg, 20%). ¹H NMR (400 MHz, CD₃OD) δ 7.31 (d, J=7.6 Hz, 1H), 7.12 (d, J=8.1 Hz, 1H), 6.33 (t, J=54.0 Hz, 1H), 5.93-5.06 (m, 2H), 4.93 (s, 2H), 4.21 (s, 1H), 3.96 (s, 2H), 3.72 (s, 3H), 3.70-3.32 (m, 3H), 3.05-2.26 (m, 3H), 1.40 (s, 6H), 1.35 (dd, J=16.3, 5.8 Hz, 6H), 1.13-0.96 (m, 3H) ppm. MS: M/e 557 (M+1)⁺.

### Compound A166: 2-(9-(2,2-difluoroethyl)-6-((2S,5R)-4-(1-(2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile

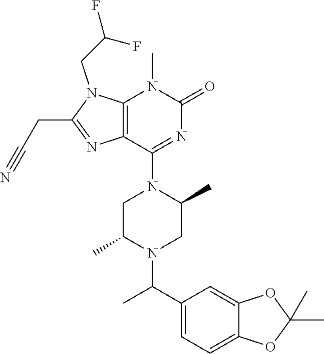 [SMILES: CC(c1ccc2c(c1)OC(C)(C)O2)N1C[C@H](C)N(c2nc(=O)n(C)c3c2nc(CC#N)n3CC(F)F)C[C@H]1C]

To a solution of 2-(9-(2,2-difluoroethyl)-6-((2S,5R)-2,5-dimethylpiperazin-1-yl)-3-methyl-2-oxo-3,9-dihydro-2H-purin-8-yl)acetonitrile (50 mg, 0.14 mmol) in CH₃CN (3 mL) and was added 1-(2,2-dimethylbenzo[d][1,3]dioxol-5-yl)ethan-1-ol (27 mg, 0.14 mmol), (cyanomethyl)trimethylphosphonium iodide (100 mg, 0.41 mmol) and DIPEA (88 mg, 0.68 mmol). The resulting mixture was stirred at 105° C. overnight. The reaction solvent was removed under reduced pressure to dryness. The resulting residue was purified by Prep-HPLC (Method A) to give the titled compound (12 mg, 16%). ¹H NMR (400 MHz, CD₃OD) δ 6.83 (s, 1H), 6.76 (s, 1H), 6.65 (t, J=8.9 Hz, 1H), 6.33 (t, J=53.8 Hz, 1H), 6.14-4.98 (m, 2H), 4.93 (s, 2H), 4.86-4.79 (m, 1H), 4.21 (s, 1H), 3.72 (s, 3H), 3.64-3.41 (m, 2H), 3.05-2.29 (m, 3H), 1.64 (s, 6H), 1.47-1.28 (m, 6H), 1.09-0.90 (m, 3H) ppm. MS: M/e 542 (M+1)⁺.

### Compound A167: 2-(6-((2S,5R)-4-(1-(2,2-difluorobenzo[d][1,3]dioxol-5-yl)ethyl)-2,5-dimethyl piperazin-1-yl)-3,9-dimethyl-2-oxo-3,9-dihydro-2H-purin-8-yl)acetonitrile

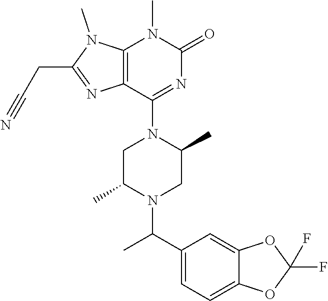 [SMILES: CC(c1ccc2c(c1)OC(F)(F)O2)N1C[C@H](C)N(c2nc(=O)n(C)c3c2nc(CC#N)n3C)C[C@H]1C]

A solution of 2-(6-((2S,5R)-2,5-dimethylpiperazin-1-yl)-3,9-dimethyl-2-oxo-3,9-dihydro-2H-purin-8-yl)acetonitrile (100 mg, 0.32 mmol), 1-(2,2-difluorobenzo[d][1,3]dioxol-5-yl)ethan-1-ol (64 mg, 0.32 mmol), (cyanomethyl)trimethylphosphonium iodide (230 mg, 0.95 mmol) and DIPEA (205 mg, 1.59 mmol) in MeCN (5 ml) was stirred at 100° C. overnight. After completed, the solution was concentrated under reduced pressure to dryness. The resulting residue containing Compound A167 was purified by Prep-HPLC (Method A) to give the titled Compound A167 (46 mg, 29%). ¹H NMR (400 MHz, CD₃OD) δ 7.29 (s, 1H), 7.21-7.08 (m, 2H), 6.18-4.89 (m, 2H), 4.21 (s, 1H), 3.93 (s, 3H), 3.78 (s, 3H), 3.68-3.48 (m, 2H), 3.29-2.52 (m, 4H), 1.59-1.32 (m, 3H), 1.31-1.21 (m, 3H), 1.05-0.88 (m, 3H) ppm. MS: M/e 500 (M+1)⁺

### Compound A168: 2-(9-ethyl-6-((2S,5R)-4-(1-(2-ethyl-2-methylbenzo[d][1,3]dioxol-5-yl)ethyl)-2,5-dimethylpiperazin-1-yl)-3-methyl-2-oxo-3,9-dihydro-2H-purin-8-yl)acetonitrile

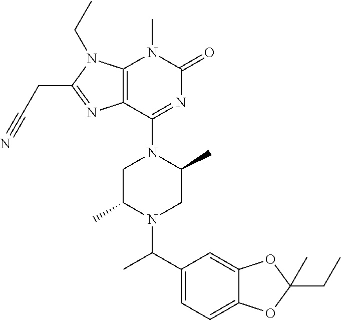 [SMILES: CCn1c(CC#N)nc2c(N3C[C@@H](C)N(C(C)c4ccc5c(c4)OC(C)(CC)O5)C[C@@H]3C)nc(=O)n(C)c21]

### Step A: 5-bromo-2-ethyl-2-methylbenzo[d][1,3]dioxole

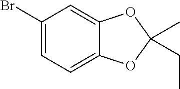

A solution of 4-bromobenzene-1,2-diol (0.5 g, 2.65 mmol), butan-2-one (381 mg, 5.29 mmol) and PBr₃ (287 mg, 1.06 mmol) in Tol. (6 ml) was stirred at 100° C. for 1 h. The solution was diluted with EtOAc (20 ml), washed with brine (10 ml), dried over Na₂SO₄ and evaporated to dryness to give the titled compound (0.64 g, 100%). ¹H NMR (400 MHz, CDCl₃) δ 6.92-6.83 (m, 2H), 6.59 (d, J=8.3 Hz, 1H), 1.94 (q, J=7.1 Hz, 2H), 1.60 (s, 3H), 1.00 (t, J=7.4 Hz, 3H) ppm.

### Step B: 1-(2-ethyl-2-methylbenzo[d][1,3]dioxol-5-yl)ethan-1-one

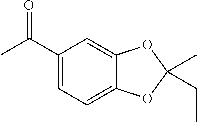

A solution of 5-bromo-2-ethyl-2-methylbenzo[d][1,3]dioxole (640 mg, 2.63 mmol), Pd(PPh₃)₂Cl₂ (92 mg, 0.13 mmol) and tributyl(1-ethoxyvinyl)stannane (1.43 g, 3.96 mmol) in Tol. (10 ml) was stirred at 100° C. overnight. The mixture was cooled to RT and HCl (4M in dioxane, 2 ml) was added, stirred for 30 min. The mixture was diluted with EtOAc (20 ml), washed with brine (10 ml), dried over Na₂SO₄ and concentrated. The residue was purified by flash column chromatography with 0%-10% EtOAc in PE to give the titled compound (160 mg, 29%).

¹H NMR (400 MHz, CDCl₃) δ 7.51 (d, J=8.8 Hz, 1H), 7.35 (s, 1H), 6.75 (d, J=7.7 Hz, 1H), 2.53 (s, 3H), 1.97 (q, J=7.3 Hz, 2H), 1.64 (s, 3H), 1.01 (t, J=7.3 Hz, 3H) ppm.

### Step C: 1-(2-ethyl-2-methylbenzo[d][1,3]dioxol-5-yl)ethan-1-ol

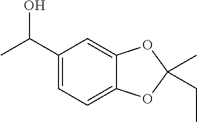

A solution of 1-(2-ethyl-2-methylbenzo[d][1,3]dioxol-5-yl)ethan-1-one (160 mg, 0.78 mmol) and NaBH₄ (29.5 mg, 0.78 mmol) in MeOH (3 ml) and THE (1 ml) was stirred at RT for 10 min. The solution was quenched with water (10 ml), diluted with EtOAc (15 ml), washed with brine (10 ml), dried and evaporated to dryness to give the titled compound (160 mg, 100%), which was used directly for the next step. MS: M/e 209 (M+1)⁺

### Step D: 2-(9-ethyl-6-((2S,5R)-4-(1-(2-ethyl-2-methylbenzo[d][1,3]dioxol-5-yl)ethyl)-2,5-dimethylpiperazin-1-yl)-3-methyl-2-oxo-3,9-dihydro-2H-purin-8-yl)acetonitrile

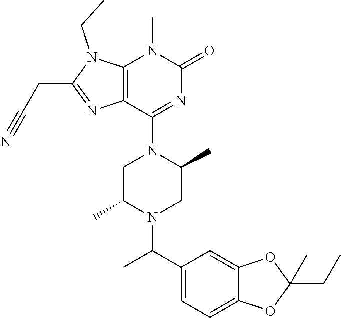 [SMILES: CCn1c(CC#N)nc2c(N3C[C@@H](C)N(C(C)c4ccc5c(c4)OC(C)(CC)O5)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (100 mg, 0.30 mmol), 1-(2-ethyl-2-methylbenzo[d][1,3]dioxol-5-yl)ethan-1-ol (160 mg, 0.77 mmol), (cyanomethyl)trimethylphosphonium iodide (222 mg, 0.91 mmol) and DIPEA (392 mg, 3.04 mmol) in MeCN (3 ml) was stirred at 100° C. overnight. The reaction was diluted with EtOAc (10 ml) and washed with brine (10 ml). The organic layer was concentrated under reduced pressure. The resulting residue was purified by flash column chromatography with 0-5% MeOH in DCM and then prep-HPLC (Method A) to give the titled compound (53 mg). ¹H NMR (400 MHz, CD₃OD) δ 6.97-6.79 (m, 2H), 6.78-6.68 (m, 1H), 6.34-5.65 (m, 1H), 5.49-5.01 (m, 1H), 4.86-4.85 (m, 3H), 4.37 (q, J=6.8 Hz, 2H), 4.24 (s, 1.5H), 3.77 (s, 4H), 3.48 (s, 1H), 3.11-2.90 (m, 0.5H), 1.96 (s, 2H), 1.60 (s, 3H), 1.56-1.40 (m, 7H), 1.35-0.96 (m, 8H) ppm. MS: M/e 520 (M+1)⁺.

### Compound A169: 2-(6-((2S,5R)-2,5-dimethyl-4-(1-(spiro[benzo[d][1,3]dioxole-2,1′-cyclobutan]-5-yl)ethyl)piperazin-1-yl)-9-ethyl-3-methyl-2-oxo-3,9-dihydro-2H-purin-8-yl)acetonitrile

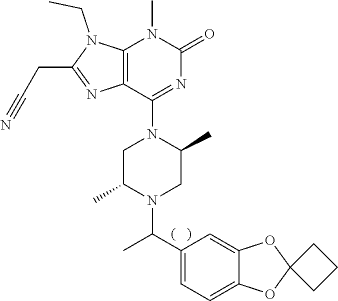

### Step A: 5-bromospiro[benzo[d][1,3]dioxole-2,1′-cyclobutane]

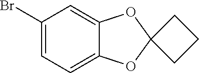 [SMILES: Brc1ccc2c(c1)OC1(CCC1)O2]

To a solution of 4-bromobenzene-1,2-diol (1.89 g, 10 mol) in toluene (8 mL) was added cyclobutanone (1.4 g, 20 mol) and PBr₃ (1.62 g, 6 mmol). The reaction mixture was sealed and stirred at 80° C. for overnight. The mixture was cooled down to RT and added H₂O. The mixture was extracted with EtOAc and then concentrated by using a rotary evaporator to give a residue. The resulting residue was purified by flash column chromatography to give the titled compound (1.7 g, 71%). MS: M/e 241 (M+1)⁺.

### Step B: 1-(spiro[benzo[d][1,3]dioxole-2,1′-cyclobutan]-5-yl)ethan-1-one

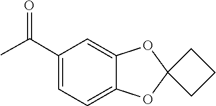

To a solution of 5-bromospiro[benzo[d][1,3]dioxole-2,1′-cyclobutane](570 mg, 2.4 mol) in toluene (10 mL) was added Pd(PPh₃)₂Cl₂ (210 mg, 0.3 mol) and tributyl(1-ethoxyvinyl)stannane (1.3 g, 3.6 mmol). The reaction mixture was protected by N₂ atmosphere and stirred at 100° C. for overnight. The mixture was added H₂O extracted with EtOAc. The organic phase was dried with Na₂SO₄ and concentrated by using a rotary evaporator to give a residue. The resulting residue was purified by flash column chromatography to give the titled compound (500 mg, 100%). MS: M/e 205 (M+1)⁺.

### Step C: 1-(spiro[benzo[d][1,3]dioxole-2,1′-cyclobutan]-5-yl)ethan-1-ol

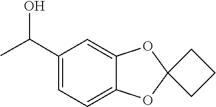

To a solution of 1-(spiro[benzo[d][1,3]dioxole-2,1′-cyclobutan]-5-yl)ethan-1-one (500 mg, 2.5 mol) in MeOH was added NaBH₄ (38 mg, 1 mmol). The reaction mixture was stirred at RT for 15 mins. The mixture was added H₂O and extracted with DCM. The organic phase was dried with Na₂SO₄ and concentrated by using a rotary evaporator to give a residue. The resulting residue was purified by flash column chromatography to give the titled compound (370 mg, 74%). MS: M/e 207 (M+1)⁺.

### Step D: 2-(6-((2S,5R)-2,5-dimethyl-4-(1-(spiro[benzo[d][1,3]dioxole-2,1′-cyclobutan]-5-yl)ethyl)piperazin-1-yl)-9-ethyl-3-methyl-2-oxo-3,9-dihydro-2H-purin-8-yl)acetonitrile

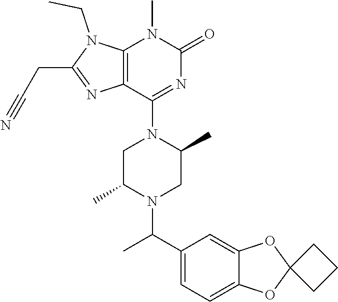 [SMILES: CCn1c(CC#N)nc2c(N3C[C@@H](C)N(C(C)c4ccc5c(c4)OC4(CCC4)O5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (480 mg, 1.5 mmol), 1-(spiro[benzo[d][1,3]dioxole-2,1′-cyclobutan]-5-yl)ethan-1-ol (450 mg, 2.2 mmol) and (cyanomethyl)trimethylphosphonium iodide (723 mg, 3 mmol) in CH₃CN (10 mL) was added DIPEA (774 mg, 6 mmol). The reaction mixture was sealed in a bottle and heated at 105° C. for 16 hours, and then cooled to room temperature, diluted with water, extracted with EtOAc. The combined organic layers were washed with brine, dried over Na₂SO₄, filtered, and concentrated to dryness. The resulting residue was purified by Prep-HPLC (Method A) to give the titled Compound A169 (200 mg, 26%), which was further separated into Compound A169a (65 mg) and Compound A169b (60 mg) by chiral Prep-HPLC. The chiral separation conditions are shown below.

| | |
|---|---|
| Column | CHIRALPAK IH |
| Column Size | 2 cm × 25 cm, 5 um |
| Mobile Phase A | MtBE |
| Mobile Phase B | EtOH |
| Flow Rate | 20 mL/min |
| Wave Length | UV 220 nm |
| Temperature | 25° C. |
| Prep-HPLC Equipment | Prep-HPLC-Gilson |

Compound A169: ¹H NMR (400 MHz, DMSO-d6) δ 6.89 (d, J=5.7 Hz, 1H), 6.79 (d, J=4.1 Hz, 2H), 6.25-5.30 (m, 1H), 5.30-4.50 (m, 1H), 4.40 (s, 2H), 4.27-4.23 (m, 2H), 3.61 (s, 3H), 3.50-3.45 (m, 1H), 3.30-3.28 (m, 1H), 2.70 (d, J=26.8 Hz, 2H), 2.60-2.55 (m, 4H), 2.33-2.18 (m, 1H), 1.79 (d, J=7.5 Hz, 2H), 1.34 (s, 4H), 1.26-1.13 (m, 5H), 0.93 (s, 1.5H), 0.81 (s, 1.5H) ppm. MS: M/e 518 (M+1)⁺.

Compound A169a (the earlier peak): ¹HNMR (400 MHz, CD₃OD) δ 6.85 (s, 1H), 6.77 (d, J=7.3 Hz, 1H), 6.68 (d, J=7.8 Hz, 1H), 6.1-5.64 (m, 1H), 5.21-4.60 (m, 1H), 4.36 (d, J=6.6 Hz, 2H), 3.76 (s, 3H), 3.60-3.40 (m, 2H), 3.30-3.13 (m, 2H), 3.00-2.65 (m, 3H), 2.62-2.58 (m, 4H), 1.90-1.80 (m, 2H), 1.46 (t, J=6.7 Hz, 6H), 1.27 (s, 3H), 0.88 (s, 3H) ppm. MS: M/e 518 (M+1)⁺.

Compound A169b (the later peak): ¹HNMR (400 MHz, CD₃OD) δ 6.85 (s, 1H), 6.76 (d, J=7.6 Hz, 1H), 6.66 (d, J=7.4 Hz, 1H), 6.10-5.60 (m, 1H), 5.25-4.50 (m, 1H), 4.37 (q, J=6.9 Hz, 2H), 3.76 (s, 4H), 3.56-3.37 (m, 2H), 3.20-3.00 (m, 1H), 2.59 (d, J=4.3 Hz, 6H), 2.26 (d, J=10.3 Hz, 1H), 1.90-1.80 (m, 2H), 1.46 (t, J=7.1 Hz, 3H), 1.29 (s, 6H), 1.02 (s, 3H) ppm. MS: M/e 518 (M+1)⁺.

### Compound A172: 2-(6-((2S,5R)-4-1-(4-chloro-2,2-dimethylbenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile

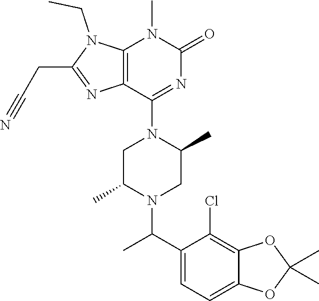 [SMILES: CCn1c(CC#N)nc2c(N3C[C@@H](C)N(C(C)c4ccc5c(c4Cl)OC(C)(C)O5)C[C@@H]3C)nc(=O)n(C)c21]

### Step A: 5-bromo-4-chloro-2,2-dimethylbenzo[d][1,3]dioxole

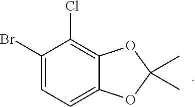

To a solution of 5-bromo-2,2-dimethylbenzo[d][1,3]dioxole (0.92 g, 4 mmol) in THE (10 mL) was added a solution of LDA in THE (2.6 ml, 5.2 mmol) drop wise at −70° C. After 1 h, a solution of 1,1,2-trichloro-1,2,2-trifluoroethane (1.12 g, 6 mmol) in THE (1 mL) was added. The reaction mixture was slowly warmed to room temperature over 16 h. The mixture was diluted with water, extracted with EtOAc (60 mL×2), washed with brine, dried over Na₂SO₄, filtered, and concentrated to dryness. The residue was purified by flash column chromatography to give the titled compound (1 g, 95%). ¹H NMR (400 MHz, CDCl₃) δ 7.07 (d, J=6.4 Hz, 1H), 6.56 (d, J=6.8 Hz, 1H), 1.74 (s, 6H) ppm.

### Step B: 1-(4-chloro-2,2-dimethylbenzo[d][1,3]dioxol-5-yl)ethan-1-one

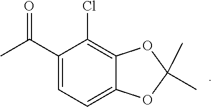

To a solution of 5-bromo-4-chloro-2,2-dimethylbenzo[d][1,3]dioxole (2.3 g, 8.77 mmol) in toluene (20 mL) was added tributyl(1-ethoxyvinyl)stannane (4.15 g, 13.2 mmol) and Pd(PPh₃)₂Cl₂ (310 mg, 0.44 mmol). The reaction mixture was protected by N₂ atmosphere and stirred at 100° C. overnight. The mixture was cooled down to RT, diluted with H₂O, extracted with EtOAc (80 mL×3), washed with brine, dried over Na₂SO₄, filtered, and concentrated to dryness. The residue was purified by flash column chromatography to give the titled compound (1.2 g, 60%). MS: M/e 227 (M+1)⁺.

### Step C: 1-(4-chloro-2,2-dimethylbenzo[d][1,3]dioxol-5-yl)ethan-1-ol

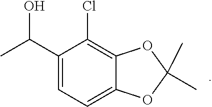

To a solution of 1-(4-chloro-2,2-dimethylbenzo[d][1,3]dioxol-5-yl)ethan-1-one (1.2 g, 5.3 mmol) in MeOH (15 mL) was added NaBH₄ (160 mg, 4.24 mmol) slowly at 0° C. The resulting mixture was stirred at room temperature for 0.5 hour. The reaction mixture was quenched with H₂O (10 mL) and extracted with EtOAc (100 mL×2), washed with brine, dried over Na₂SO₄, filtered, and concentrated to dryness. The residue was purified by flash column chromatography to give the titled compound (700 mg, 58%). ¹H NMR (400 MHz, DMSO-d6) δ 7.01 (d, J=8.0 Hz, 1H), 6.81 (d, J=8.4 Hz, 1H), 5.21 (s, 1H), 4.95-4.85 (m, 1H), 1.68 (s, 3H), 1.66 (s, 3H), 1.27 (d, J=6.0 Hz, 3H) ppm.

### Step D: 2-(6-((2S,5R)-4-1-(4-chloro-2,2-dimethylbenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile

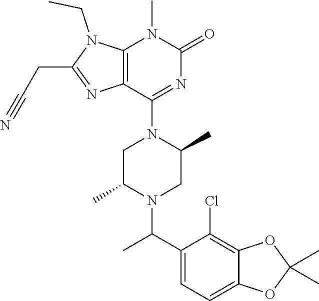 [SMILES: CCn1c(CC#N)nc2c(N3C[C@@H](C)N(C(C)c4ccc5c(c4Cl)OC(C)(C)O5)C[C@@H]3C)nc(=O)n(C)c21]

To a solution of 1-(4-chloro-2,2-dimethylbenzo[d][1,3]dioxol-5-yl)ethan-1-ol (340 mg, 1.5 mmol), 2-(6-((2S,5R)-2,5-dimethylpiperazin-1-yl)-9-ethyl-3-methyl-2-oxo-3,9-dihydro-2H-purin-8-yl)acetonitrile (450 mg, 1.5 mmol) and (cyanomethyl) trimethyl phosphonium iodide (1.09 g, 4.5 mmol) in CH₃CN (10 mL) was added DIPEA (1.9 g, 15 mmol). The mixture was sealed in a bottle and heated at 100° C. for 16 hours. The mixture was cooled to room temperature, diluted with water, extracted with EtOAc (80 mL×2), washed with brine, dried over Na₂SO₄, filtered, and concentrated to dryness. The resulting residue was purified by combi flash with 0-5% MeOH in DCM to give the titled Compound A172 (260 mg, 23%), which was further separated into Compound A172a (80 mg) and Compound A172b (73 mg) by chiral-Prep-HPLC. The chiral separation conditions are shown below.

| | |
|---|---|
| Column | CHIRALPAK IA |
| Column Size | 2 cm × 25 cm, 5 um |
| Mobile Phase A | MtBE |
| Mobile Phase B | MeOH:DCM |
| Flow Rate | 20 mL/min |
| Wave Length | UV 220 nm |
| Temperature | 25° C. |
| Prep-HPLC Equipment | Prep-HPLC-Gilson |

Compound A172a (the earlier peak): ¹H NMR (400 MHz, CD₃OD) δ 7.09 (d, J=8.0 Hz, 1H), 6.71 (d, J=8.0 Hz, 1H), 6.25-6.05 (m, 0.5H), 5.75-5.60 (m, 0.5H), 5.28-5.14 (m, 0.5H), 4.70-4.55 (m, 0.5H), 4.45-4.31 (m, 2H), 4.26-3.99 (m, 2H), 3.76 (s, 3H), 3.61-3.45 (m, 0.5H), 3.34-3.25 (m 1H), 3.27-3.14 (m, 0.5H), 3.04-2.72 (m 3H), 1.70 (s, 6H), 1.56-1.32 (m, 6H), 1.23 (d, J=6.4 Hz, 3H), 0.91 (d, J=4.8 Hz, 3H) ppm. MS: M/e 540 (M+1)⁺.

Compound A172a (the later peak): ¹H NMR (400 MHz, CD₃OD) δ 7.16 (d, J=8.4 Hz, 1H), 6.70 (d, J=8.0 Hz, 1H), 6.11-5.70 (m, 1H), 5.20-4.98 (m, 1H), 4.43-4.32 (m, 2H), 4.27-4.18 (m, 1H), 4.00-3.91 (m, 1H), 3.76 (m, 3H), 3.65-3.50 (m, 1H), 3.34-3.25 (m 2H), 2.79-2.60 (m, 1H), 2.31-2.21 (m 1H), 1.69 (s, 6H), 1.46 (t, J=6.8 Hz, 3H), 1.29 (d, J=6.4 Hz, 6H), 1.05 (d, J=5.6 Hz, 3H) ppm. MS: M/e 540 (M+1)⁺.

### Compound A173: 2-(9-ethyl-6-((2S,5R)-4-(1-(7-fluoro-2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile

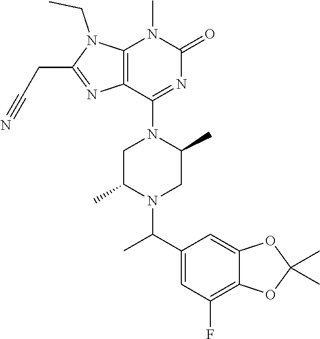 [SMILES: CCn1c(CC#N)nc2c(N3C[C@@H](C)N(C(C)c4cc(F)c5c(c4)OC(C)(C)O5)C[C@@H]3C)nc(=O)n(C)c21]

### Step A: 6-bromo-4-fluoro-2,2-dimethylbenzo[d][1,3]dioxole

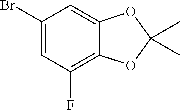

To a solution of 5-bromo-3-fluorobenzene-1,2-diol (1.65 g, 8 mmol) in toluene (10 mL) was added acetone (0.93 g, 16 mmol) and POBr₃ (2.28 g, 8 mmol). The reaction mixture was stirred at 80° C. in a sealed bottle overnight. The mixture was cooled down to RT. Weight 6 eq of Na₂CO₃, treated with ice and EtOAc. The reaction solution was added drop wise to keep the temperature below 10° C. (pH>8). extracted with EtOAc (80 mL×2), washed with brine, dried over Na₂SO₄, filtered, and concentrated to dryness. The residue was purified by flash column chromatography to give the titled compound (1.2 g, 61%). ¹H NMR (400 MHz, CDCl₃) δ 6.81 (d, J=9.6 Hz, 1H), 6.71 (s, 1H), 1.71 (s, 6H) ppm.

### Step B: 7-fluoro-2,2-dimethylbenzo[d][1,3]dioxole-5-carbaldehyde

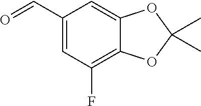

To a solution of 6-bromo-4-fluoro-2,2-dimethylbenzo[d][1,3]dioxole (1.2 g, 4.85 mmol) and 3-oxobenzo[d]isothiazole-2 (3H)-carbaldehyde 1,1-dioxide (1.54 g, 7.28 mmol) in DMF (20 mL) was added Pd(OAc)₂ (107 mg, 0.48 mmol), 1,4-Bis(diphenylphosphino)butane (413 mg, 0.97 mmol), Na₂CO₃ (1.02 g, 9.7 mmol) and Et₃SiH (675 mg, 5.82 mmol). The reaction was heated at 85° C. under N₂ overnight. Then the reaction mixture was cooled to room temperature, diluted with H₂O, extracted with EtOAc (60 mL×2), washed with brine, dried over Na₂SO₄, filtered, and concentrated to dryness. The residue was purified by flash column chromatography to give the titled compound (700 mg, crude). ¹H NMR (400 MHz, CDCl₃) δ 9.76 (s, 1H), 7.22 (d, J=8.8 Hz, 1H), 7.11 (s, 1H), 1.76 (s, 6H) ppm.

### Step C: 1-(7-fluoro-2,2-dimethylbenzo[d][1,3]dioxol-5-yl)ethan-1-ol

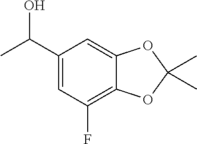

To a solution of 7-fluoro-2,2-dimethylbenzo[d][1,3]dioxole-5-carbaldehyde (670 mg, 3.4 mmol) in THE (7 mL) was added CH₃MgBr (1.2 mL, 3.6 mmol, 3 mol/L in hexane) slowly at 0° C. The resulting mixture was stirred at room temperature for 0.5 hour. The reaction mixture was quenched with H₂O (10 mL) and extracted with EtOAc (80 mL×2), washed with brine, dried over Na₂SO₄, filtered, and concentrated to dryness. The residue was purified by flash column chromatography to give the titled compound (530 mg, 51% yield for two steps). ¹H NMR (400 MHz, DMSO-d6) δ 6.72 (d, J=11.6 Hz, 1H), 6.69 (s, 1H), 5.17 (s, 1H), 4.66-4.54 (m, 1H), 1.67 (s, 6H), 1.26 (d, J=6.0 Hz, 3H) ppm.

### Step D: 2-(9-ethyl-6-((2S,5R)-4-(1-(7-fluoro-2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile

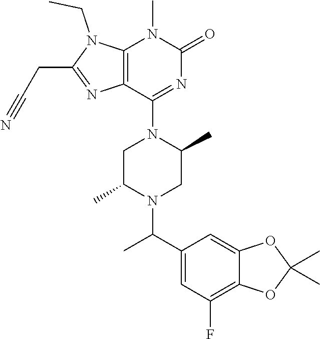 [SMILES: CCn1c(CC#N)nc2c(N3C[C@@H](C)N(C(C)c4cc(F)c5c(c4)OC(C)(C)O5)C[C@@H]3C)nc(=O)n(C)c21]

To a solution of 1-(7-fluoro-2,2-dimethylbenzo[d][1,3]dioxol-5-yl)ethan-1-ol (530 mg, 2.5 mmol), 2-(6-((2S,5R)-2,5-dimethylpiperazin-1-yl)-9-ethyl-3-methyl-2-oxo-3,9-dihydro-2H-purin-8-yl)acetonitrile (800 mg, 2.5 mmol) and (cyanomethyl) trimethyl phosphonium iodide (1.82 g, 7.5 mmol) in CH₃CN (10 mL) was added DIPEA (3.2 g, 25 mmol). The mixture was sealed in a bottle and heated at 100° C. for 16 hours. The mixture was cooled to room temperature, diluted with water, extracted with EtOAc (100 mL×2), washed with brine, dried over Na₂SO₄, filtered, and concentrated to dryness. The resulting residue was purified by combi flash (DCM/MeOH=20/1) to give the titled Compound A173 (300 mg, 23%), which was further separated into Compound A173a (110 mg) and Compound A173b (125 mg) by chiral-Prep-HPLC. The chiral separation conditions are shown below.

| | |
|---|---|
| Column | CHIRALPAK IH |
| Column Size | 2 cm × 25 cm, 5 um |
| Mobile Phase A | MtBE |
| Mobile Phase B | EtOH |
| Flow Rate | 20 mL/min |
| Wave Length | UV 220 nm |
| Temperature | 25° C. |
| Prep-HPLC Equipment | Prep-HPLC-Gilson |

Compound A173: ¹H NMR (400 MHz, CD₃OD) δ 6.74-6.62 (m, 2H), 6.19-5.65 (m, 1H), 5.31-4.95 (m, 1H), 4.43-4.31 (m, 2H), 4.26-4.18 (m, 1H), 3.76 (s, 3H), 3.60-3.44 (m, 2H), 3.35-3.31 (m, 1H), 3.01-2.57 (m, 2.5H), 2.30-2.16 (m, 0.5H), 1.68 (s, 6H), 1.53-1.39 (m, 4.5H), 1.33-1.22 (m, 4.5H), 1.21 (d, J=6.0 Hz, 1.5H), 0.90 (d, J=6.4 Hz, 1.5H) ppm. MS: M/e 524 (M+1)⁺.

Compound A173a (the earlier peak): ¹H NMR (400 MHz, DMSO-d6) δ 6.76 (d, J=11.3 Hz, 1H), 6.73 (s, 1H), 6.02-4.41 (m, 2H), 4.40-4.31 (m, 2H), 4.30-4.12 (m, 2H), 3.58 (s, 3H), 3.52-3.38 (m, 1H), 3.28-2.96 (m, 1H), 2.85-2.63 (m, 3H), 1.68-1.64 (m, 6H), 1.37-1.25 (m, 6H), 1.16 (d, J=6.4 Hz, 3H), 0.80 (d, J=6.0 Hz, 3H) ppm. MS: M/e 524 (M+1)⁺.

Compound A173b (the later peak): ¹H NMR (400 MHz, DMSO-d6) 6.75 (d, J=11.3 Hz, 1H), 6.70 (s, 1H), 5.85-4.45 (m, 2H), 4.38 (s, 2H), 4.28-4.18 (m, 2H), 3.58 (s, 3H), 3.48-3.34 (m, 2H), 3.29-3.20 (m, 1H), 2.60-2.53 (m, 1H), 2.20-2.07 (m, 1H), 1.65 (s, 6H), 1.31 (t, J=7.1 Hz, 3H), 1.24-1.07 (m, 6H), 0.95-0.85 (m, 3H) ppm. MS: M/e 524 (M+1)⁺.

### Compound A174: 2-(9-ethyl-6-((2S,5R)-4-(1-(6-fluoro-2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile

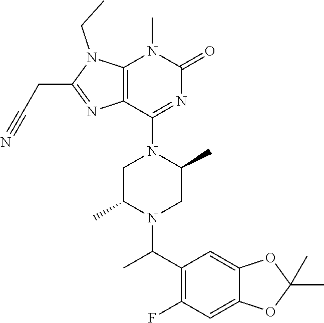 [SMILES: CCn1c(CC#N)nc2c(N3C[C@@H](C)N(C(C)c4cc5c(cc4F)OC(C)(C)O5)C[C@@H]3C)nc(=O)n(C)c21]

### Step A: 4-bromo-5-fluorobenzene-1,2-diol

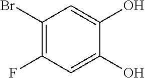

To a solution of 1-bromo-2-fluoro-4,5-dimethoxybenzene (235 mg, 1 mol) in DCM (15 mL) was added BBr₃ (1M, 4 mmol, 4 mL). The reaction mixture was stirred at RT for 2 hrs. The mixture was added H₂O and extracted with EtOAc and then concentrated by using a rotary evaporator to give a residue. The resulting residue was purified by flash column chromatography to give the titled compound (200 mg, 98%). MS: M/e 206 (M+1)⁺.

### Step B: 5-bromo-6-fluoro-2,2-dimethylbenzo[d][1,3]dioxole

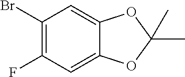

To a solution of 4-bromo-5-fluorobenzene-1,2-diol (501 mg, 2.43 mol) in toluene (5 mL) was added acetone (278 mg, 4.8 mol) and PCl₃ (137 mg, 0.97 mmol). The reaction mixture was sealed and stirred at 80° C. for overnight. The mixture was cooled down to RT and added NaHCO₃ aqueous solution. The mixture was extracted with EtOAc and then concentrated by using a rotary evaporator to give a residue. The resulting residue was purified by flash column chromatography to give the titled compound (248 mg, 42%). MS: M/e 247 (M+1)⁺.

### Step C: 1-(6-fluoro-2,2-dimethylbenzo[d][1,3]dioxol-5-yl)ethan-1-one

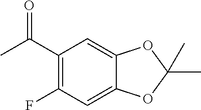

To a solution of 5-bromo-6-fluoro-2,2-dimethylbenzo[d][1,3]dioxole (120 mg, 0.5 mol) in toluene (10 mL) was added Pd(PPh₃)₂Cl₂ (7 mg, 0.01 mol) and tributyl(1-ethoxyvinyl)stannane (252 mg, 0.7 mmol). The reaction mixture was protected by N₂ atmosphere and stirred at 100° C. for overnight. The mixture was added 4N Hcl in dionxane and stirred at RT for 5 mins. The mixture was added saturated NaHCO₃ aqueous solution and extracted with EtOAc. The organic phase was dried with Na₂SO₄ and concentrated by using a rotary evaporator to give a residue. The resulting residue was purified by flash column chromatography to give the titled compound (30 mg, 29%). MS: M/e 211 (M+1)⁺.

### Step D: 1-(6-fluoro-2,2-dimethylbenzo[d][1,3]dioxol-5-yl)ethan-1-ol

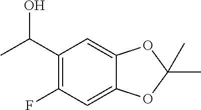

To a solution of 1-(6-fluoro-2,2-dimethylbenzo[d][1,3]dioxol-5-yl)ethan-1-one (30 mg, 0.14 mol) in MeOH was added NaBH₄ (38 mg, 1 mmol). The reaction mixture was stirred at RT for 15 mins. The mixture was added H₂O and extracted with DCM. The organic phase was dried with Na₂SO₄ and concentrated by using a rotary evaporator to give a residue. The resulting residue was purified by flash column chromatography to give the titled compound (10 mg, 33%). MS: M/e 213 (M+1)⁺.

### Step D: 2-(9-ethyl-6-((2S,5R)-4-(1-(6-fluoro-2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile

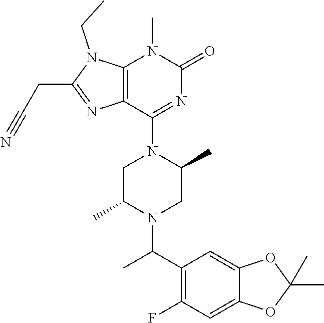 [SMILES: CCn1c(CC#N)nc2c(N3C[C@@H](C)N(C(C)c4cc5c(cc4F)OC(C)(C)O5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (30 mg, 0.14 mmol), 1-(6-fluoro-2,2-dimethylbenzo[d][1,3]dioxol-5-yl)ethan-1-ol (10 mg, 0.05 mmol) and (cyanomethyl)trimethylphosphonium iodide (120 mg, 0.5 mmol) in CH₃CN (5 mL) was added DIPEA (258 mg, 2 mmol). The reaction mixture was sealed in a bottle and heated at 105° C. for 16 hours, and then cooled to room temperature, diluted with water, extracted with EtOAc. The combined organic layers were washed with brine, dried over Na₂SO₄, filtered, and concentrated to dryness. The resulting residue was purified and separated into Compound A174a (0.7 mg) and Compound A174b (0.6 mg) by Prep-HPLC (Method B).

Compound A174a (the earlier peak): ¹H NMR (400 MHz, CD₃OD) δ 6.93 (d, J=5.2 Hz, 1H), 6.50 (d, J=9.7 Hz, 1H), 6.10-5.70 (m, 1H), 5.20-4.50 (m, 1H), 4.37 (d, J=7.1 Hz, 2H), 4.23 (s, 1H), 3.80-3.76 (m, 1H), 3.76 (s, 3H), 3.60-3.54 (m, 1H), 3.30-3.13 (m, 1H), 2.90-2.50 (m, 1H), 2.65 (s, 1H), 2.31 (d, J=12.4 Hz, 1H), 1.64 (d, J=6.4 Hz, 6H), 1.46 (t, J=6.8 Hz, 3H), 1.31 (d, J=6.4 Hz, 6H), 1.04 (d, J=5.2 Hz, 3H) ppm. MS: M/e 524 (M+1)⁺.

Compound A174b (the later peak): ¹H NMR (400 MHz, CD₃OD) δ 6.87 (d, J=5.4 Hz, 1H), 6.52 (d, J=9.5 Hz, 1H), 6.30-5.50 (m, 1H), 5.50-4.50 (m, 1H), 4.37 (d, J=7.0 Hz, 2H), 4.22 (s, 1H), 3.98 (d, J=5.8 Hz, 1H), 3.76 (s, 3H), 3.55-3.10 (m, 2H), 2.87 (d, J=36.8 Hz, 3H), 1.66 (d, J=8.7 Hz, 6H), 1.49-1.40 (m, 6H), 1.28 (d, J=6.2 Hz, 3H), 0.92 (d, J=5.5 Hz, 3H) ppm. MS: M/e 524 (M+1)⁺.

### Compound A175: 2-(9-ethyl-6-((2S,5R)-4-(1-(4-fluoro-2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile

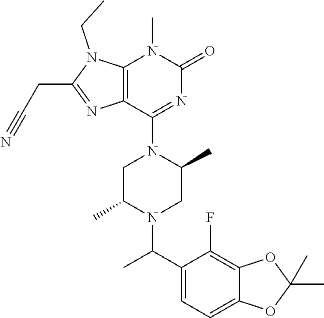 [SMILES: CCn1c(CC#N)nc2c(N3C[C@@H](C)N(C(C)c4ccc5c(c4F)OC(C)(C)O5)C[C@@H]3C)nc(=O)n(C)c21]

### Step A: 4-bromo-3-fluorobenzene-1,2-diol

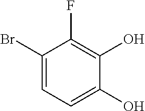

To a solution of 4-bromo-3-fluoro-2-methoxyphenol (442 mg, 2 mol) in DCM (15 mL) was added BBr₃ (1M, 4 mmol, 4 mL). The reaction mixture was stirred at RT for 2 hrs. The mixture was added H₂O and extracted with EtOAc and then concentrated by using a rotary evaporator to give a residue. The resulting residue was purified by flash column chromatography to give the titled compound (350 mg, 85%). MS: M/e 206 (M+1)⁺.

### Step B: 5-bromo-4-fluoro-2,2-dimethylbenzo[d][1,3]dioxole

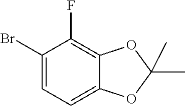

To a solution of 4-bromo-3-fluorobenzene-1,2-diol (280 mg, 1.35 mol) in toluene (5 mL) was added acetone (156 mg, 2.7 mol) and PCl₃ (95 mg, 0.72 mmol). The reaction mixture was sealed and stirred at 80° C. for overnight. The mixture was cooled down to RT and added NaHCO₃ aqueous solution. The mixture was extracted with EtOAc and then concentrated by using a rotary evaporator to give a residue. The resulting residue was purified by flash column chromatography to give the titled compound (180 mg, 54%). MS: M/e 247 (M+1)⁺.

### Step C: 1-(4-fluoro-2,2-dimethylbenzo[d][1,3]dioxol-5-yl)ethan-1-one

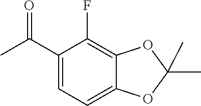

To a solution of 5-bromo-4-fluoro-2,2-dimethylbenzo[d][1,3]dioxole (180 mg, 0.7 mol) in toluene (10 mL) was added Pd(PPh₃)₂Cl₂ (49 mg, 0.07 mol) and tributyl(1-ethoxyvinyl)stannane (360 mg, 1 mmol). The reaction mixture was protected by N₂ atmosphere and stirred at 100° C. for overnight. The mixture was added 4N Hcl in dionxane and stirred at rt for 5 mins. The mixture was added saturated NaHCO₃ aqueous solution and extracted with EtOAc. The organic phase was dried with Na₂SO₄ and concentrated by using a rotary evaporator to give a residue. The resulting residue was purified by flash column chromatography to give the titled compound (120 mg, 82%). MS: M/e 211 (M+1)⁺.

### Step D: 1-(4-fluoro-2,2-dimethylbenzo[d][1,3]dioxol-5-yl)ethan-1-ol

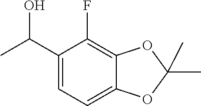

To a solution of 1-(4-fluoro-2,2-dimethylbenzo[d][1,3]dioxol-5-yl)ethan-1-one (120 mg, 0.57 mol) in MeOH was added NaBH₄ (38 mg, 1 mmol). The reaction mixture was stirred at rt for 15 mins. The mixture was added H₂O and extracted with DCM. The organic phase was dried with Na₂SO₄ and concentrated by using a rotary evaporator to give a residue. The resulting residue was purified by flash column chromatography to give the titled compound (80 mg, 67%). MS: M/e 213 (M+1)⁺.

### Step D: 2-(9-ethyl-6-((2S,5R)-4-(1-(4-fluoro-2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile

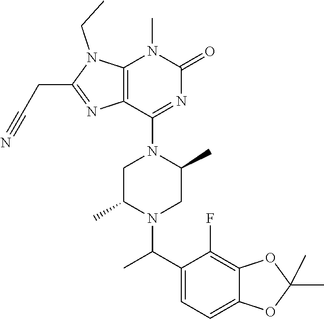 [SMILES: CCn1c(CC#N)nc2c(N3C[C@@H](C)N(C(C)c4ccc5c(c4F)OC(C)(C)O5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol), 1-(4-fluoro-2,2-dimethylbenzo[d][1,3]dioxol-5-yl)ethan-1-ol (80 mg, 0.37 mmol) and (cyanomethyl)trimethylphosphonium iodide (120 mg, 0.5 mmol) in CH₃CN (5 mL) was added DIPEA (258 mg, 2 mmol). The reaction mixture was sealed in a bottle and heated at 105° C. for 16 hours, and then cooled to room temperature, diluted with water, extracted with EtOAc. The combined organic layers were washed with brine, dried over Na₂SO₄, filtered, and concentrated to dryness. The resulting residue was purified and separated into Compound A175a (7 mg) and Compound A175b (5 mg) by Prep-HPLC (Method B).

Compound A175: ¹H NMR (400 MHz, CD₃OD) δ 6.89 (d, J=5.7 Hz, 1H), 6.79 (d, J=4.1 Hz, 1H), 5.98-5.47 (m, 1H), 5.06-4.64 (m, 1H), 4.40 (s, 2H), 4.27-4.24 (m, 2H), 3.94-3.84 (m, 1H) 3.61 (s, 3H), 3.48-3.46 (m, 1H), 3.30-3.28 (m, 1H), 2.80-2.60 (m, J=26.8 Hz, 2H), 2.23-2.18 (m, 1H), 1.79 (d, J=7.5 Hz, 6H), 1.34 (s, 4H), 1.20 (m, 5H), 0.93 (s, 1.5H), 0.81 (s, 1.5H) ppm. MS: M/e 524 (M+1)⁺.

Compound A175a (the earlier peak): ¹H NMR (400 MHz, CD₃OD) δ 6.99 (t, J=7.2 Hz, 1H), 6.57 (d, J=8.2 Hz, 1H), 6.00-5.50 (m, 1H), 5.50-5.00 (m, 1H), 4.37 (q, J=6.7 Hz, 2H), 4.23 (s, 2H), 3.84-3.82 (m, 1H), 3.76-3.74 (m, 4H), 3.58-3.56 (m, 1H), 2.70-2.68 (m, 1H), 2.33 (d, J=11.8 Hz, 1H), 1.69 (s, 6H), 1.46 (t, J=7.0 Hz, 3H), 1.35 (d, J=6.1 Hz, 3H), 1.28 (s, 3H), 1.05 (d, J=5.6 Hz, 3H) ppm. MS: M/e 524 (M+1)⁺.

Compound A175b (the later peak): ¹H NMR (400 MHz, CD₃OD) δ 6.93 (t, J=7.1 Hz, 1H), 6.59 (d, J=7.8 Hz, 1H), 6.30-5.50 (m, 1H), 5.50-4.50 (m, 1H), 4.36 (d, J=6.6 Hz, 2H), 4.22 (s, 2H), 3.94 (d, J=5.2 Hz, 1H), 3.76 (s, 3H), 3.52-3.46 (m, 1H), 2.91 (d, J=22.0 Hz, 2H), 2.78 (d, J=11.5 Hz, 1H), 1.69 (s, 6H), 1.44 (dd, J=17.1, 10.0 Hz, 6H), 1.32 (d, J=6.0 Hz, 3H), 0.92 (d, J=5.8 Hz, 3H) ppm. MS: M/e 524 (M+1)⁺.

### Compound A176: 2-(6-((2S,5R)-4-(1-(4,6-difluoro-2,2-dimethylbenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile

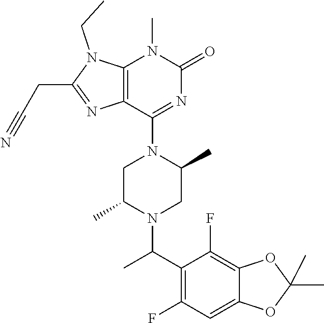 [SMILES: CCn1c(CC#N)nc2c(N3C[C@@H](C)N(C(C)c4c(F)cc5c(c4F)OC(C)(C)O5)C[C@@H]3C)nc(=O)n(C)c21]

### Step A: 3,5-difluorobenzene-1,2-diol

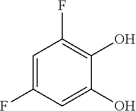

To a solution of 3,5-difluoro-2-hydroxybenzaldehyde (500 mg, 3.2 mmol) in THE (50 mL) was added dropwise aq. NaOH solution (152 mg, 3.8 mmol, 2 mL) at 0° C., and then 30% H₂O₂ solution (1.4 mL). The mixture was stirred for 2 hours at RT and followed by the addition of a second portion of 30% H₂O₂ (1.4 mL). After stirred for 4 hours, it was cooled to 0° C. and aq. NaOH solution (2 N) was added until pH=10˜11 was reached, and then the mixture was stirred for 0.5 h and quenched with cone. HCl at 0° C. to pH=2˜3. It was extracted with dichloromethane (50 mL×3) and washed with brine (30 mL×2), dried over Na₂SO₄, and concentrated to give residue. The resulting residue was purified by flash column chromatography (MeOH:DCM=0-10%) to give 3,5-difluorobenzene-1,2-diol (200 mg, 43%). MS: M/e 147 (M+1)⁺.

### Step B: 4,6-difluoro-2,2-dimethylbenzo[d][1,3]dioxole

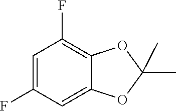

A solution of 3,5-difluorobenzene-1,2-diol (200 mg, 1.37 mmol), POBr₃ (392 mg, 1.37 mmol), acetone (159 mg, 2.74 mmol) in Tol (5 mL) was stirred at 85° C. overnight. The mixture was quenched with water (10 mL), extracted with dichloromethane (50 mL×3) and washed with brine (30 mL×2), dried over Na₂SO₄, and concentrated to give residue. The resulting residue was purified by flash column chromatography (MeOH:DCM=0-10%) to give 4,6-difluoro-2,2-dimethylbenzo[d][1,3]dioxole (50 mg, 20%). MS: M/e 187 (M+1)⁺.

### Step C: 5-bromo-4,6-difluoro-2,2-dimethylbenzo[d][1,3]dioxole

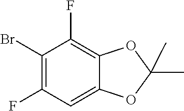

A solution of 4,6-difluoro-2,2-dimethylbenzo[d][1,3]dioxole (50 mg, 0.27 mmol), NBS (57 mg, 0.32 mmol) in DCM (5 mL) was stirred at RT overnight. The mixture was quenched with water (10 mL), extracted with dichloromethane (50 mL×3) and washed with brine (30 mL×2), dried over Na₂SO₄, and concentrated to give residue. The resulting residue was purified by flash column chromatography (MeOH:DCM=0-10%) to give 5-bromo-4,6-difluoro-2,2-dimethylbenzo[d][1,3]dioxole (70 mg, 98%). MS: M/e 265 (M+1)⁺.

### Step D: 1-(4,6-difluoro-2,2-dimethylbenzo[d][1,3]dioxol-5-yl)ethan-1-ol

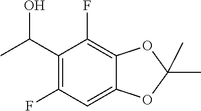 [SMILES: CC(O)c1c(F)cc2c(c1F)OC(C)(C)O2]

To a solution of 5-bromo-4,6-difluoro-2,2-dimethylbenzo[d][1,3]dioxole (70 mg, 0.26 mmol) in THE (20 mL) was added n-Butyllithium (0.2 mL, 0.32 mmol) at −78° C. and stirred for 1 h. Then acetaldehyde was added and stirred at −78° C. for 1 h. the mixture was quenched by water, extracted with dichloromethane (50 mL×3) and washed with brine (30 mL×2), dried over Na₂SO₄, and concentrated to give residue. The resulting residue was purified by flash column chromatography (MeOH:DCM=0-10%) to give 1-(4,6-difluoro-2,2-dimethylbenzo[d][1,3]dioxol-5-yl)ethan-1-ol (50 mg, 82%). MS: M/e 231 (M+1)⁺.

### Step E: 2-(6-((2S,5R)-4-(1-(4,6-difluoro-2,2-dimethylbenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile

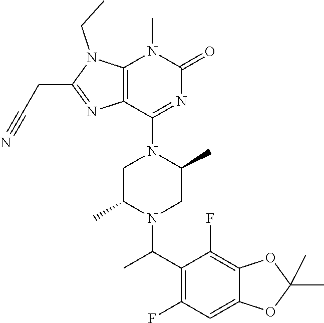 [SMILES: CCn1c(CC#N)nc2c(N3C[C@@H](C)N(C(C)c4c(F)cc5c(c4F)OC(C)(C)O5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol) in CH₃CN (3 mL) and was added 1-(4,6-difluoro-2,2-dimethylbenzo[d][1,3]dioxol-5-yl)ethan-1-ol (35 mg, 0.15 mmol), (cyanomethyl) trimethyl phosphonium iodide (111 mg, 0.46 mmol) and DIPEA (98 mg, 0.76 mmol). The resulting mixture was stirred at 105° C. overnight. The reaction solvent was removed under reduced pressure to dryness. The resulting residue was purified by Prep-TLC (DCM:MeOH=20:1) and further separated into Compound A176a (20 mg) and Compound A176b (14 mg) by Prep-HPLC (Method A).

Compound A176a (the earlier peak): ¹H NMR (400 MHz, CD₃OD) δ 6.44 (d, J=9.6 Hz, 1H), 6.14-4.89 (m, 2H), 4.37 (d, J=7.4 Hz, 2H), 4.23 (s, 1H), 3.99 (s, 1H), 3.76 (s, 3H), 3.53 (s, 1H), 3.30-3.13 (m, 2H), 2.76 (s, 1H), 2.27 (d, J=11.7 Hz, 1H), 1.69 (s, 6H), 1.47 (d, J=7.1 Hz, 6H), 1.25 (s, 3H), 0.99 (s, 3H) ppm. MS: M/e 542 (M+1)⁺.

Compound A176b (the later peak): ¹H NMR (400 MHz, CD₃OD) δ 6.47 (d, J=9.7 Hz, 1H), 5.93-5.04 (m, 2H), 4.35 (s, 2H), 4.23 (s, 1H), 4.10 (s, 1H), 3.76 (s, 3H), 3.47 (s, 2H), 2.89 (s, 2H), 2.75 (s, 1H), 1.70 (s, 6H), 1.45 (d, J=7.0 Hz, 6H), 1.37 (s, 3H), 0.95 (s, 3H) ppm. MS: M/e 542 (M+1)⁺.

### Compound A178: 2-(9-ethyl-6-((2S,5R)-4-(1-(2-isopropyl-1,3-dioxoisoindolin-5-yl)ethyl)-2,5-dimethylpiperazin-1-yl)-3-methyl-2-oxo-3,9-dihydro-2H-purin-8-yl)acetonitrile

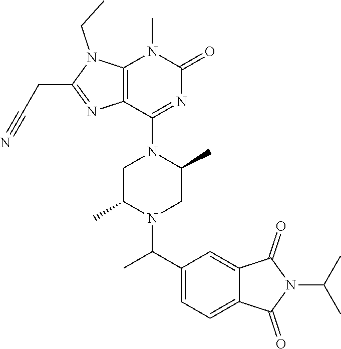 [SMILES: CCn1c(CC#N)nc2c(N3C[C@@H](C)N(C(C)c4ccc5c(c4)C(=O)N(C(C)C)C5=O)C[C@@H]3C)nc(=O)n(C)c21]

### Step A: 5-(1-hydroxyethyl)-2-isopropylisoindoline-1,3-dione

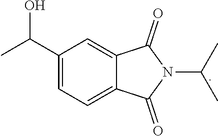 [SMILES: CC(O)c1ccc2c(c1)C(=O)N(C(C)C)C2=O]

To a solution of 5-acetyl-2-isopropylisoindoline-1,3-dione (150 mg, 0.65 mmol) in MeOH (15 mL) was added NaBH₄ (25 mg, 0.65 mmol) at room temperature and the resulting mixture was stirred at room temperature for 15 mins. The reaction mixture was diluted with DCM (200 mL). The organic layer was washed with water, dried over Na₂SO₄ and concentrated to give the titled compound (130 mg, 86%). MS: M/e 234 (M+1)⁺.

### Step B: 2-(9-ethyl-6-((2S,5R)-4-(1-(2-isopropyl-1,3-dioxoisoindolin-5-yl)ethyl)-2,5-dimethylpiperazin-1-yl)-3-methyl-2-oxo-3,9-dihydro-2H-purin-8-yl)acetonitrile

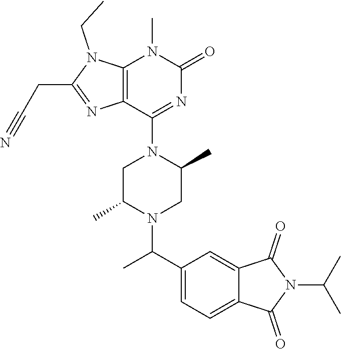 [SMILES: CCn1c(CC#N)nc2c(N3C[C@@H](C)N(C(C)c4ccc5c(c4)C(=O)N(C(C)C)C5=O)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol) in CH₃CN (3 mL) and was added 5-(1-hydroxyethyl)-2-isopropylisoindoline-1,3-dione (35 mg, 0.15 mmol), (cyanomethyl)trimethylphosphonium iodide (110 mg, 0.46 mmol) and DIPEA (98 mg, 0.76 mmol). The resulting mixture was stirred at 105° C. overnight. The reaction solvent was removed under reduced pressure to dryness. The resulting residue containing compound was purified by Prep-HPLC (Method A) to give the titled compound (32 mg, 39%). ¹H NMR (400 MHz, CD₃OD) δ 7.98 (s, 1H), 7.89 (s, 2H), 6.29-5.06 (m, 2H), 4.51 (s, 1H), 4.38 (d, J=7.1 Hz, 2H), 4.26 (s, 2H), 4.11-3.80 (m, 1H), 3.78 (s, 3H), 3.70-3.48 (m, 1H), 3.29-2.68 (m, 2H), 2.67-2.05 (m, 1H), 1.49 (s, 3H), 1.47 (d, J=6.6 Hz, 9H), 1.32 (s, 6H) ppm. MS: M/e 545 (M+1)⁺

### Compound A179: 2-(6-((2S,5R)-4-(1-(2,2-dimethyl-3-oxo-2,3-dihydrobenzofuran-6-yl)ethyl)-2,5-dimethylpiperazin-1-yl)-9-ethyl-3-methyl-2-oxo-3,9-dihydro-2H-purin-8-yl)acetonitrile

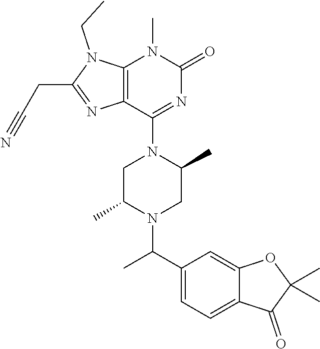 [SMILES: CCn1c(CC#N)nc2c(N3C[C@@H](C)N(C(C)c4ccc5c(c4)OC(C)(C)C5=O)C[C@@H]3C)nc(=O)n(C)c21]

### Step A: 1-(4-bromo-2-fluorophenyl)-2-methylpropan-2-ol

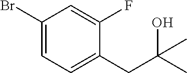

To a solution of methyl 2-(4-bromo-2-fluorophenyl) acetate (2 g, 8.10 mmol) in THE (20 ml) at −60° C. under N₂ was added MeMgBr (3M, 6.75 ml, 20.25 mmol). The solution was warmed to rt naturally and stirred for 1 h. The solution was diluted with EtOAc (30 ml), washed with brine (10 ml), dried and concentrated. The residue was purified by flash column chromatography with 0%-20% EtOAc in PE to give the titled compound (1.7 g, 85%). MS: M/e 247 (M+1)⁺.

### Step B: 6-bromo-2,2-dimethyl-2,3-dihydrobenzofuran

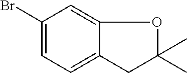

A solution of 1-(4-bromo-2-fluorophenyl)-2-methylpropan-2-ol (1.7 g, 6.91 mmol) and tBuOK (1M in THF, 8.2 ml, 8.2 mmol) in THE (20 ml) was stirred at 60° C. for 1 h. The solution was diluted with EtOAc (30 ml), washed with brine (10 ml), dried and evaporated to dryness to give the titled compound (1.4 g, 90%), which was used directly for the next step.

### Step C: 6-bromo-2,2-dimethylbenzofuran-3 (2H)-one

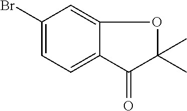

A solution of 6-bromo-2,2-dimethyl-2,3-dihydrobenzofuran (1.2 g, 5.29 mmol), K₂S₂O₈ (4.3 g, 15.93 mmol) and CuSO₄ (0.85 g, 5.31 mmol) in MeCN (12 ml) and H₂O (12 ml) was stirred at 100° C. for 1 h. The solution was diluted with EtOAc (30 ml), washed with brine (15 ml×2), died and then concentrated. The residue was purified by flash column chromatography with 0%-5% EtOAc in PE to give the titled compound (1 g, 78%). MS: M/e 241,243 (M+1)⁺.

### Step D: 6-acetyl-2,2-dimethylbenzofuran-3 (21H)-one

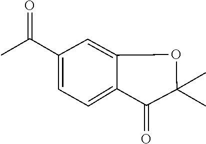

A solution of 6-bromo-2,2-dimethylbenzofuran-3 (2H)-one (500 mg, 2.07 mmol), Pd(PPh₃)₂Cl₂ (73 mg, 0.10 mmol) and tributyl(1-ethoxyvinyl)stannane (1.1 g, 3.05 mmol) in Tol. (8 ml) was stirred at 100° C. overnight. The mixture was cooled to RT and HCl (4M in dioxane, 2 ml) was added, stirred for 30 min. The mixture was diluted with EtOAc (20 ml), washed with brine (10 ml), dried and concentrated. The residue was purified by flash column chromatography with 0%-15% MEA in PE to give the titled compound (400 mg, 95%). ¹H NMR (400 MHz, CDCl₃) δ 7.75 (d, J=7.8 Hz, 1H), 7.63 (d, J=8.4 Hz, 2H), 2.65 (s, 3H), 1.49 (s, 6H) ppm.

### Step E: 6-(1-hydroxyethyl)-2,2-dimethylbenzofuran-3 (2H)-one

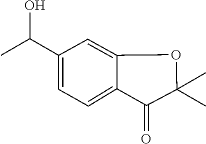

A solution of 6-acetyl-2,2-dimethylbenzofuran-3 (2H)-one (100 mg, 0.49 mmol) and NaBH₄ (9.3 mg, 0.24 mmol) in MeOH (2 ml) was stirred at RT for 10 min. The solution was quenched with water (10 ml), diluted with EtOAc (15 ml), washed with brine (10 ml), dried and evaporated to dryness to give the titled compound (100 mg, 100%), which was used directly for the next step. ¹H NMR (400 MHz, CDCl₃) δ 7.64 (d, J=7.3 Hz, 1H), 7.12 (s, 1H), 7.06 (d, J=7.8 Hz, 1H), 4.96 (q, J=6.4 Hz, 1H), 1.52 (d, J=6.1 Hz, 3H), 1.47 (s, 6H) ppm.

### Step F: 2-(6-((2S,5R)-4-(1-(2,2-dimethyl-3-oxo-2,3-dihydrobenzofuran-6-yl)ethyl)-2,5-dimethylpiperazin-1-yl)-9-ethyl-3-methyl-2-oxo-3,9-dihydro-2H-purin-8-yl)acetonitrile

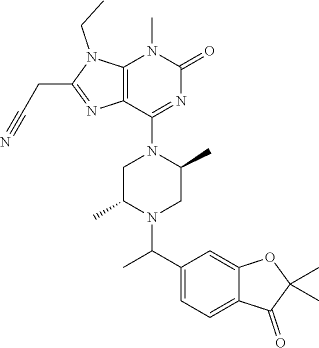 [SMILES: CCn1c(CC#N)nc2c(N3C[C@@H](C)N(C(C)c4ccc5c(c4)OC(C)(C)C5=O)C[C@@H]3C)nc(=O)n(C)c21]

A solution of 2-(6-((2S,5R)-2,5-dimethylpiperazin-1-yl)-9-ethyl-3-methyl-2-oxo-3,9-dihydro-2H-purin-8-yl)acetonitrile (100 mg, 0.30 mmol), 6-(1-hydroxyethyl)-2,2-dimethylbenzofuran-3 (2H)-one (100 mg, 0.48 mmol), (cyanomethyl)trimethylphosphonium iodide (222 mg, 0.91 mmol) and DIPEA (392 mg, 3.04 mmol) in MeCN (3 ml) was stirred at 100° C. overnight. The reaction was diluted with EtOAc (10 ml) and washed with brine (10 ml). The organic layer was concentrated under reduced pressure. The resulting residue was purified by flash column chromatography with 0-5% MeOH in DCM and then Prep-HPLC (Method A) to give the titled compound (14 mg). ¹H NMR (400 MHz, DMSO-d6) δ 7.62 (t, J=8.2 Hz, 1H), 7.23 (d, J=11.1 Hz, 2H), 6.13-5.46 (m, 1H), 5.20-4.47 (m, 1H), 4.43 (d, J=18.5 Hz, 2H), 4.26 (d, J=7.3 Hz, 2H), 3.71 (d, J=5.5 Hz, 0.5H), 3.61 (s, 3H), 3.59-3.47 (m, 1H), 3.31 (s, 1.5H), 2.80 (s, 1.5H), 2.13 (d, J=11.3 Hz, 0.5H), 1.40 (s, 6H), 1.34 (s, 4.5H), 1.26 (dd, J=13.7, 5.9 Hz, 3H), 1.20 (s, 1.5H), 0.97 (s, 1.5H), 0.86 (s, 1.5H) ppm. MS: M/e 518 (M+1)⁺.

### Compound A181: 2-(6-((2S,5R)-4-(1-(3,3-dimethyl-2,3-dihydrofuro[3,2-b]pyridin-5-yl)ethyl)-2,5-dimethylpiperazin-1-yl)-9-ethyl-3-methyl-2-oxo-3,9-dihydro-2H-purin-8-yl)acetonitrile

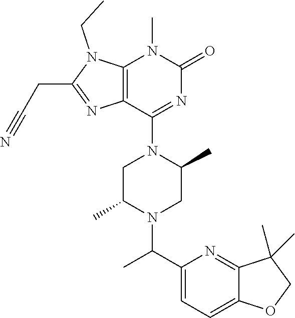 [SMILES: CCn1c(CC#N)nc2c(N3C[C@@H](C)N(C(C)c4ccc5c(n4)C(C)(C)CO5)C[C@@H]3C)nc(=O)n(C)c21]

### Step A: 5-hydroxy-6-iodopicolinic acid

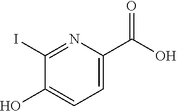 [SMILES: O=C(O)c1ccc(O)c(I)n1]

To a stirred solution of 5-hydroxypyridine-2-carboxylic acid (1 g, 6.8 mmol, 95%) in NH₃·H₂O (30 mL) were added a solution of KI (5.97 g, 34 mmol) and 12 (1.82 g, 6.8 mmol) in H₂O (40 mL) dropwise at room temperature. The resulting mixture was stirred for overnight at room temperature. The mixture was acidified to pH=3 with HCl (1M). The resulting mixture was extracted with EtOAc (3×150 mL). The combined water layers were concentrated under reduced pressure to give the titled compound (1 g, 55%). MS: M/e 266 (M+1)⁺.

### Step B: methyl 5-hydroxy-6-iodopicolinate

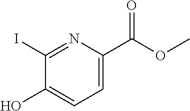

To a stirred solution of 5-hydroxy-6-iodopyridine-2-carboxylic acid (1 g, 3.7 mmol) in MeOH was added SOCl₂ (1.3 mL, 18 mmol) dropwise at 0° C. The resulting mixture was stirred for 2 hours at 70° C. The mixture was allowed to cool down to room temperature. The resulting mixture was concentrated under reduced pressure. The residue was purified by reverse flash chromatography to give the titled compound (600 mg, 53%). MS: M/e 280 (M+1)⁺.

### Step C: methyl 6-iodo-5-((2-methylallyl)oxy)picolinate

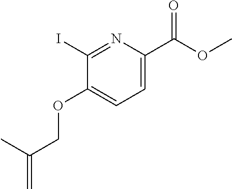 [SMILES: C=C(C)COc1ccc(C(=O)OC)nc1I]

A solution of methyl 5-hydroxy-6-iodopyridine-2-carboxylate (500 mg, 1.6 mmol), 3-bromo-2-methylprop-1-ene (250 mg, 1.8 mmol) and K₂CO₃ (609 mg, 4.1 mmol) in acetone was stirred at 60° C. overnight under nitrogen atmosphere. The resulting mixture was concentrated under reduced pressure. The residue was purified by flash column chromatography to give the titled compound (500 mg, 89%). MS: M/e 334 (M+1)⁺.

### Step D: methyl 3,3-dimethyl-2,3-dihydrofuro[3,2-b]pyridine-5-carboxylate

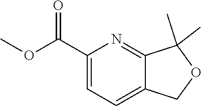

A solution of methyl 6-iodo-5-[(2-methylprop-2-en-1-yl)oxy]pyridine-2-carboxylate (500 mg, 1.5 mmol), n-Bu₃SnH (689 mg, 2.25 mmol) and AIBN (26 mg, 0.15 mmol) in benzene was stirred at 80° C. overnight under nitrogen atmosphere. The mixture was allowed to cool down to room temperature. The resulting mixture was added KF (10%, 5 mL) at room temperature. The resulting mixture was stirred for 3.5 hours at room temperature. The resulting mixture was extracted with EtOAc (3×50 mL). The combined organic layers were dried over anhydrous Na₂SO₄. After filtration, the filtrate was concentrated under reduced pressure. The residue was purified by flash column chromatography to give the titled compound (240 mg, 77%). MS: M/e 208 (M+1)⁺.

### Step E: 3,3-dimethyl-2,3-dihydrofuro[3,2-b]pyridine-5-carboxylic acid

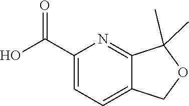

To a solution of methyl 3,3-dimethyl-2,3-dihydrofuro[3,2-b]pyridine-5-carboxylate (0.2 g, 0.97 mmol) in MeOH (4 mL)/water (1 mL) was added LiOH·H₂O (58 mg, 1.5 mmol). The mixture solution was stirred at room temperature for 5 hours. The reaction was quenched with water and acidified to pH=5˜6 with citric acid, extracted with DCM (20 mL×2). The combined organic layers were washed with brine, dried over Na₂SO₄ and concentrated to give the titled compound (0.18 g, 96%). MS: M/e 194 (M+1)⁺.

### Step F: N-methoxy-N, 3,3-trimethyl-2,3-dihydrofuro[3,2-b]pyridine-5-carboxamide

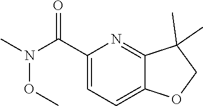

To a solution of 3,3-dimethyl-2,3-dihydrofuro[3,2-b]pyridine-5-carboxylic acid (0.14 g, 0.72 mmol) in DMF (2 mL) were added HATU (548 mg, 1.4 mmol), DIPEA (465 mg, 3.6 mol) and N,N-dimethyl-hydroxylamine hydrochloride salt (210 mg, 2.2 mol). The mixture was stirred at room temperature for overnight. The reaction was diluted with DCM and washed with water. The organic layer was separated, dried by Na₂SO₄, filtered, and concentrated to dryness. The resulting residue was purified by flash column chromatography to give the titled compound (120 mg, 70%). MS: M/e 237 (M+1)⁺.

### Step G: 1-(3,3-dimethyl-2,3-dihydrofuro[3,2-b]pyridin-5-yl)ethan-1-one

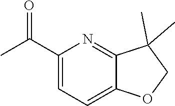

A mixture of N-methoxy-N, 3,3-trimethyl-2,3-dihydrofuro[3,2-b]pyridine-5-carboxamide (120 mg, 0.51 mmol) in THE (5 mL) at 0° C. was added methyl magnesium bromide (0.33 mL, 3M, 1.02 mol). The reaction mixture was stirred at room temperature for 30 mins. The reaction was quenched by adding aqueous NH₄Cl. The resulting mixture was extracted with EtOAc and then concentrated by using a rotary evaporator, to give the titled compound (110 mg). MS: M/e 192 (M+1)⁺.

### Step H: 1-(3,3-dimethyl-2,3-dihydrofuro[3,2-b]pyridin-5-yl)ethan-1-ol

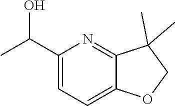

To a solution of 1-(3,3-dimethyl-2,3-dihydrofuro[3,2-b]pyridin-5-yl)ethan-1-one (110 mg, 0.57 mmol) in MeOH (5 mL) was added NaBH₄ (21 mg, 0.57 mmol) at room temperature and the resulting mixture was stirred at room temperature for 5 min. The reaction mixture was diluted with DCM and washed with water, dried over Na₂SO₄ and concentrated to give the titled compound (100 mg). MS: M/e 194 (M+1)⁺.

### Step I: 2-(6-((2S,5R)-4-(1-(3,3-dimethyl-2,3-dihydrofuro[3,2-b]pyridin-5-yl)ethyl)-2,5-dimethylpiperazin-1-yl)-9-ethyl-3-methyl-2-oxo-3,9-dihydro-2H-purin-8-yl)acetonitrile

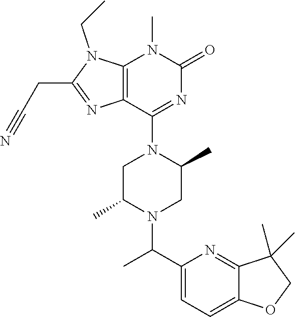 [SMILES: CCn1c(CC#N)nc2c(N3C[C@@H](C)N(C(C)c4ccc5c(n4)C(C)(C)CO5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol) in CH₃CN (2 mL) and was added 1-(3,3-dimethyl-2,3-dihydrofuro[3,2-b]pyridin-5-yl)ethan-1-ol (44 mg, 0.23 mmol), (cyanomethyl)trimethylphosphonium iodide (110 mg, 0.45 mmol) and DIPEA (194 mg, 1.5 mmol). The resulting mixture was stirred at 105° C. overnight. The reaction solvent was removed under reduced pressure. The resulting residue was purified to give the titled compound (7 mg) by Prep-HPLC (Method A). ¹H NMR (400 MHz, CD₃OD) δ 7.33 (d, J=8.4 Hz, 1H), 7.14 (dd, J=8.3, 3.5 Hz, 1H), 6.21-4.89 (m, 2H), 4.39 (d, J=7.0 Hz, 1H), 4.35 (s, 3H), 4.22 (s, 1H), 3.94-3.81 (m, 1H), 3.77 (s, 3H), 3.73-3.32 (m, 2H), 3.08-2.10 (m, 3H), 1.51-1.24 (m, 15H), 1.21-0.89 (m, 3H) ppm. MS: M/e 505 (M+1)⁺.

### Compound A183: 2-(6-((2S,5R)-4-(1-(3,3-dimethyl-2,3-dihydrobenzofuran-5-yl)ethyl)-2,5-dimethylpiperazin-1-yl)-9-ethyl-3-methyl-2-oxo-3,9-dihydro-2H-purin-8-yl)acetonitrile

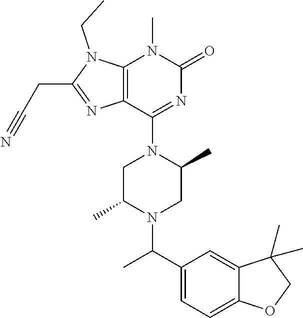 [SMILES: CCn1c(CC#N)nc2c(N3C[C@@H](C)N(C(C)c4ccc5c(c4)C(C)(C)CO5)C[C@@H]3C)nc(=O)n(C)c21]

### Step A: 1-bromo-2-((2-methylallyl)oxy)benzene

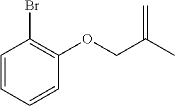

To a solution of 2-bromophenol (2.457 g, 14.2 mol) in actone (75 mL) was added 3-bromo-2-methylprop-1-ene (2 ml, 14.9 mol) and K₂CO₃ (4.9 g, 35 mmol). The reaction mixture was protected by N₂ atmosphere and stirred at 70° C. for overnight. The mixture was cooled down to RT and added H₂O. The mixture was extracted with EtOAc then concentrated by using a rotary evaporator to give a residue. The resulting residue was purified by flash column chromatography to give the titled compound (2.6 g, 81%). MS: M/e 227 (M+1)⁺.

### Step B: 3,3-dimethyl-2,3-dihydrobenzofuran

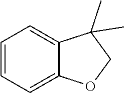

To a solution of 1-bromo-2-((2-methylallyl)oxy)benzene (1.1 g, 4.8 mol) in toluene (15 mL) was added Bu₃SnH (2.2 g, 7.6 mmol) and AIBN (80 mg, 0.48 mmol). The reaction mixture was protected by N₂ atmosphere and stirred at 100° C. for overnight. The mixture was cooled down to RT and added H₂O. The mixture was extracted with EtOAc then concentrated by using a rotary evaporator to give a residue. The resulting residue was purified by flash column chromatography to give the titled compound (0.7 g, 99%). MS: M/e 149 (M+1)⁺

### Step C: 5-bromo-3,3-dimethyl-2,3-dihydrobenzofuran

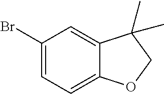

To a solution of 3,3-dimethyl-2,3-dihydrobenzofuran (0.7 g, 4.69 mol) in DCM (15 mL) was added NBS (1.7 g, 7 mmol). The reaction mixture was stirred at RT for 2 h. The mixture was added H₂O and extracted with EtOAc then concentrated by using a rotary evaporator to give a residue. The resulting residue was purified by flash column chromatography to give the titled compound (0.6 g, 60%). MS: M/e 227 (M+1)⁺

### Step D: 1-(3,3-dimethyl-2,3-dihydrobenzofuran-5-yl)ethan-1-one

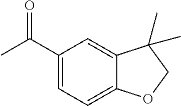

To a solution of 5-bromo-3,3-dimethyl-2,3-dihydrobenzofuran (200 mg, 0.88 mol) in toluene (10 mL) was added Pd(PPh₃)₂Cl₂ (70 mg, 0.1 mol) and tributyl(1-ethoxyvinyl)stannane (476 mg, 1.32 mmol). The reaction mixture was protected by N₂ atmosphere and stirred at 90° C. for overnight. The mixture was added 4N Hcl in dionxane and stirred at RT for 15 mins. The mixture was added saturated NaHCO₃ aqueous solution and extracted with EtOAc. The organic phase was dried with Na₂SO₄ and concentrated by using a rotary evaporator to give a residue. The resulting residue was purified by flash column chromatography to give the titled compound (100 mg, 59.8%). MS: M/e 191 (M+1)⁺.

### Step E: 1-(3,3-dimethyl-2,3-dihydrobenzofuran-5-yl)ethan-1-ol

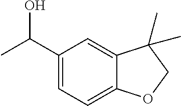

To a solution of 1-(3,3-dimethyl-2,3-dihydrobenzofuran-5-yl)ethan-1-one (100 mg, 0.5 mol) in MeOH was added NaBH₄ (38 mg, 1 mmol). The reaction mixture was stirred at RT for 15 mins. The mixture was added H₂O and extracted with DCM. The organic phase was dried with Na₂SO₄ and concentrated by using a rotary evaporator to give a residue. The resulting residue was purified by flash column chromatography to give the titled compound (50 mg, 50%). MS: M/e 193 (M+1)⁺.

### Step F: 2-(6-((2S,5R)-4-(1-(3,3-dimethyl-2,3-dihydrobenzofuran-5-yl)ethyl)-2,5-dimethylpiperazin-1-yl)-9-ethyl-3-methyl-2-oxo-3,9-dihydro-2H-purin-8-yl)acetonitrile

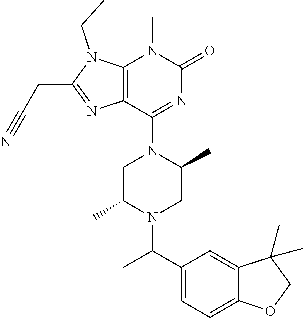 [SMILES: CCn1c(CC#N)nc2c(N3C[C@@H](C)N(C(C)c4ccc5c(c4)C(C)(C)CO5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.14 mmol), 1-(3,3-dimethyl-2,3-dihydrobenzofuran-5-yl)ethan-1-ol (50 mg, 0.26 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EtOAc. The combined organic layers were washed with brine, dried over Na₂SO₄, filtered, and concentrated to dryness. The resulting residue was purified by flash column chromatography to give the titled compound (16 mg, 24%). ¹H NMR (400 MHz, CD₃OD) δ 7.21 (d, J=13.7 Hz, 1H), 7.11 (dd, J=29.3, 8.1 Hz, 1H), 6.69 (dd, J=19.4, 8.1 Hz, 1H), 5.92 (s, 1H), 4.89 (s, 1H), 4.37 (q, J=7.1 Hz, 2H), 4.22 (d, J=4.6 Hz, 3H), 3.76 (s, 3H), 3.65 (s, 1H), 3.48 (s, 1H), 3.13-2.89 (m, 2H), 2.69 (s, 1H), 2.31 (s, 1H), 1.46 (t, J=7.1 Hz, 4H), 1.31 (m, 11H), 1.07 (d, J=6.3 Hz, 1.5H), 0.95 (d, J=5.4 Hz, 1.5H) ppm. MS: M/e 507 (M+1)⁺.

### Compound A184: 2-(6-((2S,5R)-4-(1-(3,3-dimethyl-2,3-dihydro-1H-inden-5-yl)ethyl)-2,5-dimethylpiperazin-1-yl)-9-ethyl-3-methyl-2-oxo-3,9-dihydro-2H-purin-8-yl)acetonitrile

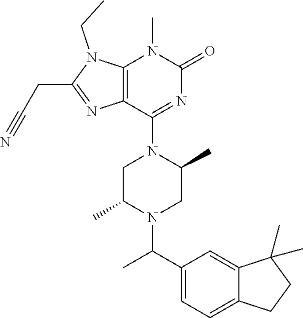 [SMILES: CCn1c(CC#N)nc2c(N3C[C@@H](C)N(C(C)c4ccc5c(c4)C(C)(C)CC5)C[C@@H]3C)nc(=O)n(C)c21]

### Step A: 1-(3,3-dimethyl-2,3-dihydro-1H-inden-5-yl)ethan-1-one

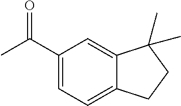

To a solution of 6-bromo-1,1-dimethyl-2,3-dihydro-1H-indene (220 mg, 1 mol) in toluene (10 mL) was added Pd(PPh₃)₂Cl₂ (70 mg, 0.1 mol) and tributyl(1-ethoxyvinyl)stannane (541 mg, 1.5 mmol). The reaction mixture was protected by N₂ atmosphere and stirred at 100° C. for overnight. The mixture was added 4N HCl in dionxane and stirred at RT for 15 mins. The mixture was added saturated NaHCO₃ aqueous solution and extracted with EtOAc. The organic phase was dried with Na₂SO₄ and concentrated by using a rotary evaporator to give a residue. The resulting residue was purified by flash column chromatography to give the titled compound (150 mg, 79%). MS: M/e 189 (M+1)⁺.

### Step B: 1-(3,3-dimethyl-2,3-dihydro-1H-inden-5-yl)ethan-1-ol

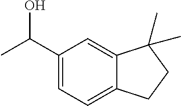

To a solution of 1-(3,3-dimethyl-2,3-dihydro-1H-inden-5-yl)ethan-1-one (150 mg, 0.79 mol) in MeOH was added NaBH₄ (38 mg, 1 mmol). The reaction mixture was stirred at RT for 15 mins. The mixture was added H₂O and extracted with DCM. The organic phase was dried with Na₂SO₄ and concentrated by using a rotary evaporator to give a residue. The resulting residue was purified by flash column chromatography to give the titled compound (70 mg, 47%). MS: M/e 191 (M+1)⁺.

### Step C: 2-(6-((2S,5R)-4-(1-(3,3-dimethyl-2,3-dihydro-1H-inden-5-yl)ethyl)-2,5-dimethylpiperazin-1-yl)-9-ethyl-3-methyl-2-oxo-3,9-dihydro-2H-purin-8-yl)acetonitrile

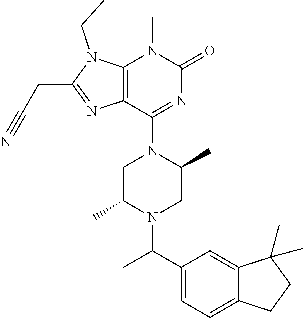 [SMILES: CCn1c(CC#N)nc2c(N3C[C@@H](C)N(C(C)c4ccc5c(c4)C(C)(C)CC5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.15 mmol), 1-(3,3-dimethyl-2,3-dihydro-1H-inden-5-yl)ethan-1-ol (100 mg, 0.5 mmol) and (cyanomethyl)trimethylphosphonium iodide (160 mg, 0.66 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EtOAc. The combined organic layers were washed with brine, dried over Na₂SO₄, filtered, and concentrated to dryness. The resulting residue was purified by flash column chromatography to give the titled compound (6 mg, 8%). ¹H NMR (400 MHz, DMSO-d6) δ 7.20 (d, J=21.1 Hz, 1H), 7.15 (s, 1H), 7.09 (s, 1H), 5.93 (s, 1H), 5.07 (s, 1H), 4.37 (q, J=7.1 Hz, 2H), 4.21 (s, 1H), 3.78 (d, J=14.2 Hz, 4H), 3.63 (s, 1H), 3.48 (s, 2H), 2.87 (dd, J=13.2, 6.8 Hz, 3H), 2.65-2.27 (m, 1H), 1.93 (dd, J=10.9, 6.9 Hz, 2H), 1.46 (t, J=7.1 Hz, 4H), 1.35 (s, 3H), 1.25 (d, J=7.8 Hz, 8H), 1.06 (d, J=5.4 Hz, 1.5H), 0.92 (s, 1.5H) ppm. MS: M/e 502 (M+1)⁺.

### Compound A185: 2-(9-ethyl-6-((2S,5R)-4-(1-(7-fluoro-2,2-dimethylbenzo[d][1,3]dioxol-4-yl)ethyl)-2,5-dimethylpiperazin-1-yl)-3-methyl-2-oxo-3,9-dihydro-2H-purin-8-yl)acetonitrile

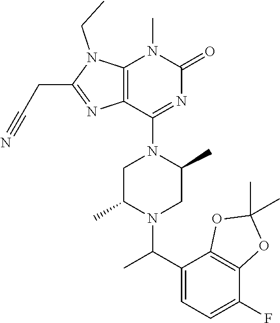 [SMILES: CCn1c(CC#N)nc2c(N3C[C@@H](C)N(C(C)c4ccc(F)c5c4OC(C)(C)O5)C[C@@H]3C)nc(=O)n(C)c21]

### Step A: 3-bromo-6-fluoro-2-hydroxybenzaldehyde

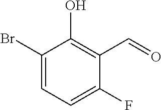 [SMILES: O=Cc1c(F)ccc(Br)c1O]

A mixture of 2-bromo-5-fluorophenol (19.1 g, 100 mmol), MgCl₂ (19.0 g, 200 mmol), Et₃N (20.2 g, 200 mmol) and paraformaldehyde (9.0 g, 300 mmol) in THF (150 mL) was stirred at 85° C. for 24 hours. An aqueous solution of HCl (2M) was added to pH˜3. The mixture was extracted with EtOAc (200 mL×3). The combined extracts was washed with brine (200 mL×3), dried over Na₂SO₄, filtered and concentrated to dryness. The resulted residue was purified by flash column chromatography to give a light yellow suspension. PE (30 mL) was added and stirred for 10 minutes. The suspension was filtered and the filter cake was washed with PE (20 mL), dried under high vacuum to give the titled compound (5.2 g, 24%). ¹H NMR (400 MHz, DMSO-d₆) δ 11.87 (s, 1H), 10.17 (s, 1H), 7.95 (t, J=7.2 Hz, 1H), 6.90 (t, J=9.6 Hz, 1H) ppm.

### Step B: 3-bromo-6-fluorobenzene-1,2-diol

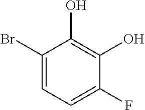

To a suspension of 3-bromo-6-fluoro-2-hydroxybenzaldehyde (5.2 g, 23.7 mmol) in H₂O (40 mL) was added H₂O₂ (3.5 g, 30%, 30.8 mmol) at 0° C. and followed by aqueous solution of NaOH (3M, 10.3 mL, 30.8 mmol) in drops. The resulted mixture was stirred at 0° C. for 30 min and room temperature for 30 min. The mixture was acidified with HCl (2.5 M) to Ph-3 and extracted with EtOAc (50 mL×2). The extracts were combined and washed with brine (50 mL×2), dried over Na₂SO₄, filtered and concentrated to dryness. The resulted residue was purified by flash column chromatography to give the titled compound (4.6 g, 94%). ¹H NMR (400 MHz, DMSO-d₆) δ 9.83 (s, 1H), 9.66 (s, 1H), 6.94 (t, J=6.8 Hz, 1H), 6.65 (t, J=9.6 Hz, 1H) ppm.

### Step C: 4-bromo-7-fluoro-2,2-dimethylbenzo[d][1,3]dioxole

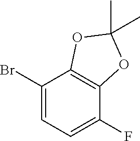

To a mixture of 3-bromo-6-fluorobenzene-1,2-diol (1.04 g, 5.0 mmol) and acetone (580 mg, 10.0 mmol) in toluene (8 mL) was added POBr₃ (1.43 g, 5.0 mmol) and the resulted mixture was stirred at 80° C. for 20 hours. The mixture was cooled and diluted with EtOAc (20 mL), basified with aqueous solution of NaHCO₃ to Ph-8, washed with brine (20 mL×2), dried over Na₂SO₄, filtered and concentrated to dryness. The resulted residue was purified by column chromatography to give the titled compound (618 mg, 50%). ¹H NMR (400 MHz, CDCl₃) δ 6.93-6.80 (m, 1H), 6.56 (t, J=9.2 Hz, 1H), 1.77 (s, 6H) ppm.

### Step D: 1-(7-fluoro-2,2-dimethylbenzo[d][1,3]dioxol-4-yl)ethan-1-ol

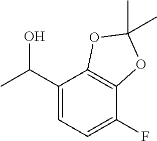

To a −70° C. solution of 4-bromo-7-fluoro-2,2-dimethylbenzo[d][1,3]dioxole (100 mg, 0.40 mmol) in THF (2 mL) was added n-BuLi (1.6 M, 0.35 mL, 1.56 mmol) in drops under N₂. After stirring for 10 minutes at −70° C., a solution of acetaldehyde (52 mg, 1.2 mmol) in THF (0.5 mL) was added. Then the mixture was allowed warm to room temperature and stirred for 2 hours. Aqueous solution of NH₄Cl (10 mL) was added and the mixture was extracted with EtOAc (5 mL×3), the organics were combined and washed with brine (10 mL×2), dried over Na₂SO₄, filtered and concentrated to dryness. The resulted residue was purified by column chromatography to give the titled compound (76 mg, 89%). ¹H NMR (400 MHz, DMSO-d₆) δ 6.97-6.90 (m, 1H), 6.80 (t, J=9.6 Hz, 1H), 5.25 (s, 1H), 4.85-4.75 (m, 1H), 1.74 (s, 6H), 1.34 (d, J=6.0 Hz, 3H) ppm.

### Step E: 2-(9-ethyl-6-((2S,5R)-4-(1-(7-fluoro-2,2-dimethylbenzo[d][1,3]dioxol-4-yl)ethyl)-2,5-dimethylpiperazin-1-yl)-3-methyl-2-oxo-3,9-dihydro-2H-purin-8-yl)acetonitrile

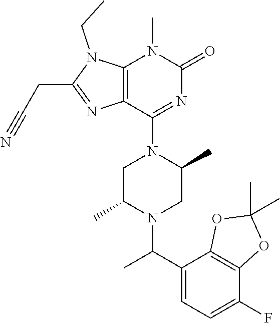 [SMILES: CCn1c(CC#N)nc2c(N3C[C@@H](C)N(C(C)c4ccc(F)c5c4OC(C)(C)O5)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (60 mg, 0.18 mmol), 1-(7-fluoro-2,2-dimethylbenzo[d][1,3]dioxol-4-yl)ethan-1-ol (60 mg, 0.28 mmol) and (cyanomethyl)trimethylphosphonium iodide (130 mg, 0.55 mmol) in CH₃CN (1 mL) was added DIPEA (93 mg, 0.72 mmol). The mixture was stirred at 100° C. in a sealed tube for 16 hours. The mixture was cooled and diluted with EtOAc (5 mL), washed with brine (5 mL×2), dried over Na₂SO₄, filtered and concentrated under reduced pressure to dryness. The resulting residue was purified by flash column chromatography and further separated into to give Compound 185a (3.5 mg) and Compound A185b (4.0 mg) by Prep-HPLC (Method A).

Compound A185a (the earlier peak): ¹H NMR (400 MHz, CD₃OD) δ 6.95-6.88 (m, 1H), 6.63 (t, J=9.4 Hz, 1H), 6.20-5.70 (m, 1H), 5.20-4.50 (m, 1H), 4.37 (q, J=6.8 Hz, 2H), 4.23 (s, 1H), 3.90-3.36 (m, 6H), 3.29-3.07 (m, 1H), 2.80-2.55 (m, 1H), 2.31 (d, J=11.6 Hz, 1H), 1.69 (s, 6H), 1.46 (t, J=6.8 Hz, 3H), 1.37-1.22 (m, 6H), 1.02 (d, J=5.2 Hz, 3H) ppm. MS: M/e 524 (M+1)⁺.

Compound 185b (the later peak): ¹H NMR (400 MHz, CD₃OD) δ 6.96-6.79 (m, 1H), 6.66 (t, J=9.6 Hz, 1H), 6.30-4.95 (m, 2H), 4.37 (d, J=7.6 Hz, 2H), 4.26-4.15 (m, 1H), 3.86-3.70 (m, 4H), 3.64-3.32 (m, 2H), 3.00-2.84 (m, 2H), 2.81-2.71 (m, 1H), 1.71 (s, 3H), 1.68 (s, 3H), 1.46 (t, J=6.8 Hz, 3H), 1.40 (d, J=5.6 Hz, 3H), 1.32 (d, J=6.0 Hz, 3H), 0.93 (d, J=6.0 Hz, 3H) ppm. MS: M/e 524 (M+1)⁺.

### Compound A188: 2-(7-((2S,5R)-4-(1-(2,2-dimethylbenzo[d][1,3]dioxol-5-yl)ethyl)-2,5-dimethylpiperazin-1-yl)-3-ethyl-6-fluoro-4-methyl-5-oxo-4,5-dihydro-3H-imidazo[4,5-b]pyridin-2-yl)acetonitrile

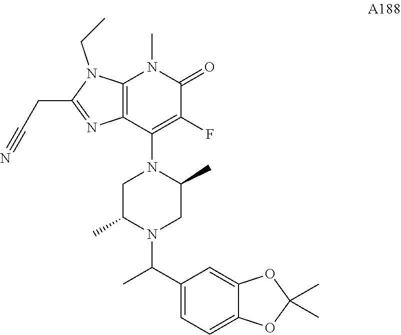 [SMILES: CCn1c(CC#N)nc2c(N3C[C@@H](C)N(C(C)c4ccc5c(c4)OC(C)(C)O5)C[C@@H]3C)c(F)c(=O)n(C)c21]

To a solution of 2-(7-((2S,5R)-2,5-dimethylpiperazin-1-yl)-3-ethyl-6-fluoro-4-methyl-5-oxo-4,5-dihydro-3H-imidazo[4,5-b]pyridin-2-yl)acetonitrile (50 mg, 0.14 mmol) in CH₃CN (2 mL) and was added 1-(2,2-dimethylbenzo[d][1,3]dioxol-5-yl)ethan-1-ol (54 mg, 0.28 mmol), (cyanomethyl)trimethylphosphonium iodide (110 mg, 0.45 mmol) and DIPEA (194 mg, 1.5 mmol). The resulting mixture was stirred at 105° C. overnight. The reaction solvent was removed under reduced pressure. The resulting residue was purified to give the titled compound (2 mg) by Prep-HPLC (Method A). ¹H NMR (400 MHz, CD₃OD) δ 6.85-6.72 (m, 2H), 6.69-6.61 (m, 1H), 4.75-4.55 (m, 1H), 4.45-4.37 (m, 2H), 4.08-3.89 (m, 1H), 3.88-3.86 (m, 3H), 3.70-3.65 (m, 2H), 3.55-3.34 (m, 2H), 3.17-2.76 (m, 2H), 2.75-2.20 (m, 1H), 1.66-1.59 (m, 6H), 1.48-1.43 (m, 3H), 1.40-1.25 (m, 6H), 1.21-0.99 (m, 3H) ppm. MS: M/e 523 (M+1)⁺.

### Compound A189: 2-(3-ethyl-6-fluoro-7-((2S,5R)-4-(1-(7-fluoro-2,2-dimethylbenzo[d][1,3]dioxol-5-yl)ethyl)-2,5-dimethylpiperazin-1-yl)-4-methyl-5-oxo-4,5-dihydro-3H-imidazo[4,5-b]pyridin-2-yl)acetonitrile

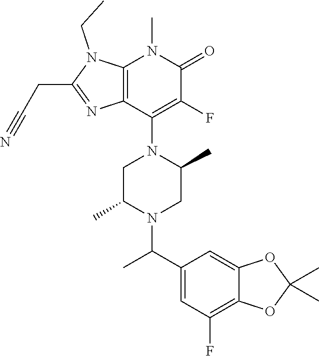 [SMILES: CCn1c(CC#N)nc2c(N3C[C@@H](C)N(C(C)c4cc(F)c5c(c4)OC(C)(C)O5)C[C@@H]3C)c(F)c(=O)n(C)c21]

To a solution of 2-(7-((2S,5R)-2,5-dimethylpiperazin-1-yl)-3-ethyl-6-fluoro-4-methyl-5-oxo-4,5-dihydro-3H-imidazo[4,5-b]pyridin-2-yl)acetonitrile (50 mg, 0.14 mmol) in CH₃CN (2 mL) and was added 1-(7-fluoro-2,2-dimethylbenzo[d][1,3]dioxol-5-yl)ethan-1-ol (61 mg, 0.28 mmol), (cyanomethyl)trimethylphosphonium iodide (105 mg, 0.43 mmol) and DIPEA (185 mg, 1.5 mmol). The resulting mixture was stirred at 105° C. overnight. The reaction solvent was removed under reduced pressure. The resulting residue was purified by flash column chromatography to give the titled compound (crude), which was further purified to give the titled compound (4 mg) by Prep-HPLC (Method A). ¹H NMR (400 MHz, CD₃OD) δ 6.75-6.63 (m, 2H), 4.79-4.55 (m, 1H), 4.45-4.37 (m, 2H), 4.26-4.20 (m, 1H), 4.10-3.89 (m, 1H), 3.87 (s, 3H), 3.79-3.40 (m, 3H), 3.10-2.73 (m, 2H), 2.65-2.17 (m, 1H), 1.73-1.65 (m, 6H), 1.50-1.37 (m, 4H), 1.33-1.23 (m, 5H), 1.20-0.99 (m, 3H) ppm. MS: M/e 541 (M+1)⁺.

### Compound A190: 2-(3-ethyl-6-fluoro-7-((2S,5R)-4-(1-(4-fluoro-2,2-dimethylbenzo[d][1,3]dioxol-5-yl)ethyl)-2,5-dimethylpiperazin-1-yl)-4-methyl-5-oxo-4,5-dihydro-3H-imidazo[4,5-b]pyridin-2-yl)acetonitrile

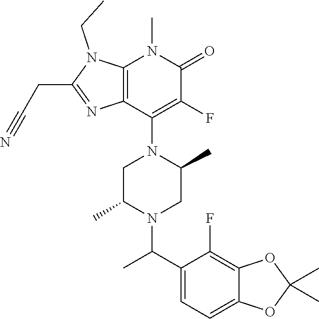 [SMILES: CCn1c(CC#N)nc2c(N3C[C@@H](C)N(C(C)c4ccc5c(c4F)OC(C)(C)O5)C[C@@H]3C)c(F)c(=O)n(C)c21]

To a solution of 2-(7-((2S,5R)-2,5-dimethylpiperazin-1-yl)-3-ethyl-6-fluoro-4-methyl-5-oxo-4,5-dihydro-3H-imidazo[4,5-b]pyridin-2-yl)acetonitrile (50 mg, 0.14 mmol) in CH₃CN (2 mL) and was added 1-(4-fluoro-2,2-dimethylbenzo[d][1,3]dioxol-5-yl)ethan-1-ol (61 mg, 0.28 mmol), (cyanomethyl)trimethylphosphonium iodide (110 mg, 0.45 mmol) and DIPEA (194 mg, 1.5 mmol). The resulting mixture was stirred at 105° C. overnight. The reaction solvent was removed under reduced pressure and purified by flash column chromatography (DCM:MeOH) to give the titled Compound A190, which was further separated into Compound A190a (22 mg) and Compound A190b (11 mg) by Prep-HPLC (Method A) and Prep-chiral HPLC. The chiral separation conditions are shown below.

| | |
|---|---|
| Column | IE |
| Column Size | 20.0 mm × 250 mm, 5 um |
| Mobile Phase A | Hex |
| Mobile Phase B | EtOH (0.2% 2M NH₃ MeOH) |
| Flow Rate | 18 mL/min |
| Gradient | Mobile Phase A:Mobile Phase B = 80:20(v/v) |
| Wave Length | UV 200 nm and 290 nm |
| Temperature | RT |
| Prep-HPLC Equipment | Prep-HPLC-Gilson |

Compound A190: ¹H NMR (400 MHz, CD₃OD) δ 7.01-6.91 (m, 1H), 6.65-6.51 (m, 1H), 4.74-4.53 (m, 1H), 4.45-4.38 (m, 2H), 3.98-4.10 (m, 1H), 3.91 (d, J=11.6 Hz, 1H), 3.87 (d, J=1.7 Hz, 3H), 3.81-3.74 (m, 1H), 3.49-3.40 (m, 2H), 3.18-2.78 (m, 2H), 2.69-2.21 (m, 1H), 1.74-1.65 (m, 6H), 1.49-1.25 (m, 9H), 1.22-1.01 (m, 3H) ppm. MS: M/e 541 (M+1)⁺.

Compound A190a (the earlier peak): ¹H NMR (400 MHz, CD₃OD) δ 6.97-6.88 (m, 1H), 6.58 (d, J=8.2 Hz, 1H), 4.74-4.65 (m, 1H), 4.42 (q, J=7.2 Hz, 2H), 4.05 (q, J=6.5 Hz, 1H), 3.87 (s, 3H), 3.75-3.67 (m, 2H), 3.37-3.33 (m, 2H), 3.10-3.01 (m, 1H), 2.88-2.79 (m, 1H), 2.67-2.57 (m, 1H), 1.69 (s, 6H), 1.45 (t, J=7.2 Hz, 3H), 1.35 (d, J=6.5 Hz, 6H), 1.04 (d, J=6.5 Hz, 3H) ppm. MS: M/e 541 (M+1)⁺.

Compound A190b (the later peak): ¹H NMR (400 MHz, CD₃OD) δ 7.03-6.93 (m, 1H), 6.56 (t, J=6.8 Hz, 1H), 4.67-4.57 (m, 1H), 4.41 (q, J=7.2 Hz, 2H), 4.03 (d, J=12.9 Hz, 1H), 3.951-3.88 (m, 2H), 3.87 (s, 3H), 3.47-3.33 (m, 3H), 2.85 (d, J=11.8 Hz, 1H), 2.24 (d, J=11.9 Hz, 1H), 1.68 (s, 6H), 1.45 (t, J=7.2 Hz, 3H), 1.33 (t, J=7.0 Hz, 3H), 1.28 (d, J=6.6 Hz, 3H), 1.14 (d, J=6.5 Hz, 3H) ppm. MS: M/e 541 (M+1)⁺.

### Compound A191: 2-(6-((2S,5R)-2,5-dimethyl-4-(1-(2-methyl-4-oxo-4H-chromen-7-yl)ethyl)piperazin-1-yl)-9-ethyl-3-methyl-2-oxo-3,9-dihydro-2H-purin-8-yl)acetonitrile

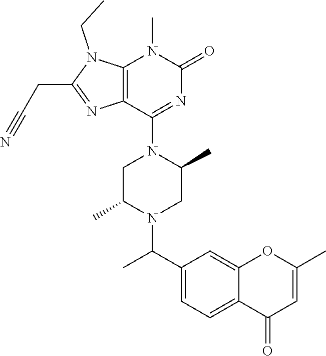 [SMILES: CCn1c(CC#N)nc2c(N3C[C@@H](C)N(C(C)c4ccc5c(=O)cc(C)oc5c4)C[C@@H]3C)nc(=O)n(C)c21]

### Step A: 1-(4-bromo-2-hydroxyphenyl)butane-1,3-dione

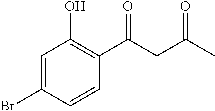

To a solution of 1-(4-bromo-2-hydroxyphenyl)ethan-1-one (2.15 g, 10 mol) in THE (15 mL) was added ethyl acetate (2.2 g, 25 mol) and NaH (60% in oil, 2 g, 50 mmol). The reaction mixture was protected by N₂ atmosphere and stirred at 70° C. for overnight. The mixture was cooled down to RT and added H₂O, The mixture was adjusted PH 2-3 with 1 N HCl solution and extracted with EtOAc then concentrated by using a rotary evaporator to give a residue. The resulting residue was purified by flash column chromatography to give the titled compound (1.8 g, 70%). MS: M/e 257 (M+1)⁺.

### Step B: 7-bromo-2-methyl-4H-chromen-4-one

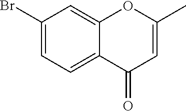

To a solution of 1-(4-bromo-2-hydroxyphenyl)butane-1,3-dione (1.8 g, 7 mol) in MeOH (15 mL) was added concentrated H₂SO₄ (1 mL). The reaction mixture was stirred at 50° C. for overnight. The mixture was cooled down to RT and added H₂O, The mixture was added H₂O and extracted with EtOAc then concentrated by using a rotary evaporator to give a residue. The resulting residue was purified by flash column chromatography to give the titled compound (1.5 g, 90%). MS: M/e 239 (M+1)⁺

### Step C: 7-acetyl-2-methyl-4H-chromen-4-one

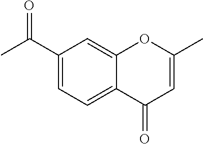

To a solution of 7-bromo-2-methyl-4H-chromen-4-one (240 mg, 1 mol) in toluene (10 mL) was added Pd(PPh₃)₂Cl₂ (70 mg, 0.1 mol) and tributyl(1-ethoxyvinyl)stannane (720 mg, 2 mmol). The reaction mixture was protected by N₂ atmosphere and stirred at 90° C. for overnight. The mixture was added 4N HCl in dionxane and stirred at RT for 15 mins. The mixture was added saturated NaHCO₃ aqueous solution and extracted with EtOAc. The organic phase was dried with Na₂SO₄ and concentrated by using a rotary evaporator to give a residue. The resulting residue was purified by flash column chromatography to give the titled compound (180 mg, 89%). MS: M/e 203 (M+1)⁺.

### Step D: 7-(1-hydroxyethyl)-2-methyl-4H-chromen-4-one

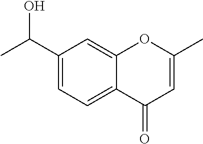

To a solution of 7-acetyl-2-methyl-4H-chromen-4-one (180 mg, 0.89 mol) in MeOH was added NaBH₄ (38 mg, 1 mmol). The reaction mixture was stirred at RT for 15 mins. The mixture was added H₂O and extracted with DCM. The organic phase was dried with Na₂SO₄ and concentrated by using a rotary evaporator to give a residue. The resulting residue was purified by flash column chromatography to give the titled compound (120 mg, 66%). MS: M/e 205 (M+1)⁺.

### Step E: 2-(6-((2S,5R)-2,5-dimethyl-4-(1-(2-methyl-4-oxo-4H-chromen-7-yl)ethyl)piperazin-1-yl)-9-ethyl-3-methyl-2-oxo-3,9-dihydro-2H-purin-8-yl)acetonitrile

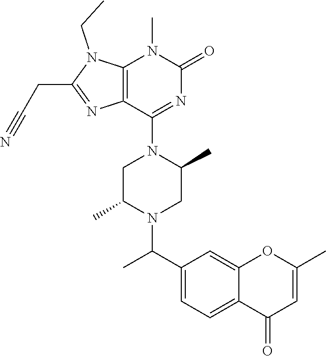 [SMILES: CCn1c(CC#N)nc2c(N3C[C@@H](C)N(C(C)c4ccc5c(=O)cc(C)oc5c4)C[C@@H]3C)nc(=O)n(C)c21]

To a solution of 2-(6-((2S,5R)-2,5-dimethylpiperazin-1-yl)-9-ethyl-3-methyl-2-oxo-3,9-dihydro-2H-purin-8-yl)acetonitrile (50 mg, 0.14 mmol), 7-(1-hydroxyethyl)-2-methyl-4H-chromen-4-one (60 mg, 0.29 mmol) and (cyanomethyl)trimethylphosphonium iodide (120 mg, 0.5 mmol) in CH₃CN (10 mL) was added DIPEA (258 mg, 2 mmol). The reaction mixture was sealed in a bottle and heated at 105° C. for 16 hours, and then cooled to room temperature, diluted with water, extracted with EtOAc. The combined organic layers were washed with brine, dried over Na₂SO₄, filtered, and concentrated to dryness. The resulting residue was purified by flash column chromatography to give the titled compound (16 mg, 24%). ¹H NMR (400 MHz, CD₃OD)) δ 8.07 (t, J=9.5 Hz, 1H), 7.61 (s, 1H), 7.56 (s, 1H), 6.24 (s, 1H), 6.10-5.50 (m, 1H), 5.50-5.00 (m, 1H), 4.37 (d, J=6.7 Hz, 2H), 4.23 (s, 2H), 3.79 (d, J=22.7 Hz, 4H), 3.68 (d, J=7.2 Hz, 1H), 2.90 (s, 2H), 2.73 (s, 0.5H), 2.45 (s, 3H), 2.19 (d, J=12.5 Hz, 0.5H), 1.47 (d, J=7.1 Hz, 4.5H), 1.38 (dd, J=12.1, 6.4 Hz, 3H), 1.30 (s, 1.5H), 1.08 (d, J=5.5 Hz, 1.5H), 0.94 (d, J=5.4 Hz, 1.5H) ppm. MS: M/e 507 (M+1)⁺.

### Compound A194: 2-(7-((2S,5R)-2,5-dimethyl-4-(1-(quinoxalin-6-yl)ethyl)piperazin-1-yl)-4-methyl-5-oxo-4,5-dihydrothiazolo[5,4-b]pyridin-2-yl)acetonitrile

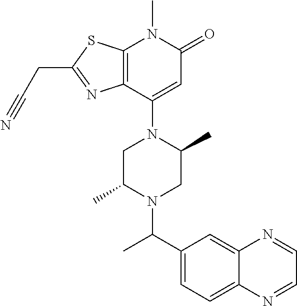 [SMILES: CC(c1ccc2nccnc2c1)N1C[C@H](C)N(c2cc(=O)n(C)c3sc(CC#N)nc23)C[C@H]1C]

### Step A: methyl 5-(methylamino)thiazole-4-carboxylate

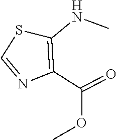

Methyl 2-isocyanoacetate (50 g, 0.5 mol) and isothiocyanatomethane (37 g, 0.5 mol) were dissolved in THF (800 ml) followed by addition of t-BuOK (1M in THF, 650 ml, 0.65 mol) dropwise. The mixture was stirred at rt overnight. The solvent was removed under reduced pressure and water was added to quench the reaction. The reaction was adjusted pH to 7 with saturated citric acid solution and extracted with EtOAc. The organic phase was washed with brine, dried (Na₂SO₄), and concentrated under reduced pressure. The resulting residue was purified by flash column chromatography (EtOAc:PE=0-70% in 50 minutes) to give the titled compound (43 g, 50%). MS: M/e 173 (M+1)⁺.

### Step B: methyl 2-bromo-5-(methylamino)thiazole-4-carboxylate

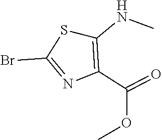

NBS (45 g, 0.25 mol) was added to the mixture of methyl 5-(methylamino)thiazole-4-carboxylate (43 g, 0.25 mol) in MeCN (300 ml). The reaction was stirred at rt for overnight. The reaction was purified by c.c (EtOAc:PE=0-50% in 20 minutes) to give the titled compound (48 g, 77%). ¹H NMR (400 MHz, CDCl₃) δ 7.38 (s, 1H), 3.94-3.81 (m, 3H), 3.03 (s, 3H) ppm. MS: M/e 251 (M+1)⁺.

### Step C: methyl 2-bromo-5-(N-methylacetamido)thiazole-4-carboxylate

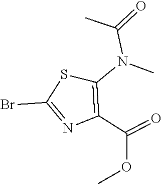

Methyl 2-bromo-5-(methylamino)thiazole-4-carboxylate (25 g, 0.1 mol) was dissolved in DCE (100 ml) followed by addition of acetic anhydride (51 g, 0.5 mol). The reaction was stirred at 100° C. for 3 days in a sealed tube. The reaction was quenched with NaHCO₃ (aq.) The aqueous phase was extracted with DCM. The combined organic layers were washed with brine, dried over MgSO₄, and concentrated to dryness. The resulting residue was slurried from PE to give the titled compound (27 g, 93%). ¹H NMR (400 MHz, CDCl₃) δ 3.94 (s, 3H), 3.33 (d, J=72.1 Hz, 3H), 2.37-1.94 (m, 3H) ppm. MS: M/e 293 (M+1)⁺.

### Step D: 2-bromo-7-hydroxy-4-methylthiazolo[5,4-b]pyridin-5 (4H)-one

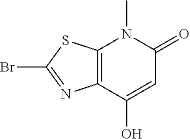

LiHMDS (1M in THF, 25 ml) was added to the mixture of methyl 2-bromo-5-(N-methylacetamido)thiazole-4-carboxylate (5 g, 17 mmol) in THF (200 ml) at −65° C. dropwise. The reaction was stirred at −60° C. to rt for 2 hours. Water was added to quench the reaction and extracted with EtOAc. The aqueous phase was adjusted pH to 3-4 with saturated citric acid solution and extracted with DCM. The organic phase was then washed with brine, dried (Na₂SO₄), and concentrated under reduced pressure to give the titled compound (1.5 g, crude). MS: M/e 261 (M+1)⁺.

### Step E: 2-bromo-4-methyl-5-oxo-4,5-dihydrothiazolo[5,4-b]pyridin-7-yl trifluoromethanesulfonate

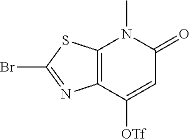 [SMILES: Cn1c(=O)cc(OS(=O)(=O)C(F)(F)F)c2nc(Br)sc21]

The 2-bromo-7-hydroxy-4-methylthiazolo[5,4-b]pyridin-5 (4H)-one (2.6 g, crude) was dissolved in the mixture of DMF/THF (20 ml/20 ml) and K₂CO₃ (2.7 g, 20 mmol) and N,N-bis(trifluoromethylsulfonyl) aniline (7.1 g, 20 mmol) was added to the solution. The reaction was stirred at rt overnight. Water was added to quench the reaction and extracted with EtOAc. The organic phase was then washed with brine, dried (Na₂SO₄), and concentrated under reduced pressure. The residue was purified by column chromatography on silica gel (petroleum ether/ethyl acetate, 0-80% in 20 minutes) to give the titled compound (600 mg, 9% over 2 steps). MS: M/e 393 (M+1)⁺.

### Step F: tert-butyl (2R,5S)-4-(2-bromo-4-methyl-5-oxo-4,5-dihydrothiazolo[5,4-b]pyridin-7-yl)-2,5-dimethylpiperazine-1-carboxylate

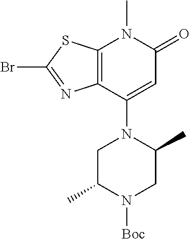 [SMILES: C[C@@H]1CN(c2cc(=O)n(C)c3sc(Br)nc23)[C@@H](C)CN1C(=O)OC(C)(C)C]

A sealed tube was charged with 2-bromo-4-methyl-5-oxo-4,5-dihydrothiazolo[5,4-b]pyridin-7-yl trifluoromethanesulfonate (700 mg, 1.8 mmol), tert-butyl (2R,5S)-2,5-dimethylpiperazine-1-carboxylate (458 mg, 2.1 mmol), DIPEA (464 mg, 3.6 mmol) and acetonitrile (10 ml). The mixture was stirred at 90° C. for overnight, cooled to rt. The reaction was purified by flash column chromatography (EtOAc:PE=0-80% in 20 minutes) to give the titled compound (650 mg, 80%). MS: M/e 457 (M+1)⁺.

### Step G: methyl 7-((2S,5R)-4-(tert-butoxycarbonyl)-2,5-dimethylpiperazin-1-yl)-4-methyl-5-oxo-4,5-dihydrothiazolo[5,4-b]pyridine-2-carboxylate

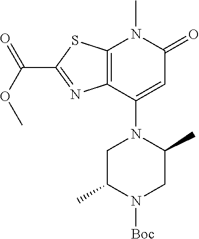 [SMILES: COC(=O)c1nc2c(N3C[C@@H](C)N(C(=O)OC(C)(C)C)C[C@@H]3C)cc(=O)n(C)c2s1]

To a mixture of tert-butyl (2R,5S)-4-(2-bromo-4-methyl-5-oxo-4,5-dihydrothiazolo[5,4-b]pyridin-7-yl)-2,5-dimethylpiperazine-1-carboxylate (650 mg, 1.4 mmol) was dissolved in MeOH (10 ml) added Pd(dppf)Cl₂ (208 mg, 0.28 mmol), Et₃N (283 mg, 2.8 mmol). The reaction was stirred at 90° C. for overnight under carbon monoxide (20 atm) atmosphere. The reaction was quenched with water and extracted with EtOAc. The combined organic layers were washed with brine, dried over Na₂SO₄, and concentrated. The residue was purified by flash column chromatography (EtOAc:PE=0-100% in 20 minutes) to give the titled compound (470 mg, 77%). MS: M/e 437 (M+1)⁺.

### Step H: tert-butyl (2R,5S)-4-(2-(hydroxymethyl)-4-methyl-5-oxo-4,5-dihydrothiazolo[5,4-b]pyridin-7-yl)-2,5-dimethylpiperazine-1-carboxylate

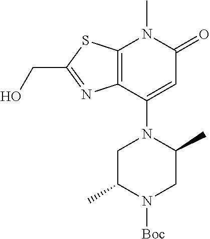 [SMILES: C[C@@H]1CN(c2cc(=O)n(C)c3sc(CO)nc23)[C@@H](C)CN1C(=O)OC(C)(C)C]

To a mixture of methyl 7-((2S,5R)-4-(tert-butoxycarbonyl)-2,5-dimethylpiperazin-1-yl)-4-methyl-5-oxo-4,5-dihydrothiazolo[5,4-b]pyridine-2-carboxylate (470 mg, 1.1 mmol) in THF (10 mL) was added NaBH₄ (164 mg, 4.3 mmol). The resulting mixture was stirred at 70° C. for overnight. The reaction was diluted with water, extracted with EtOAc, washed with brine, dried over Na₂SO₄, filtered, and concentrated. The residue was purified by flash column chromatography (MeOH:DCM=0-10% in 20 minutes) to give the titled compound (390 mg, 88%). ¹H NMR (400 MHz, CD₃OD) δ 5.74 (s, 1H), 5.49 (s, 1H), 4.89 (s, 1H), 4.83 (s, 2H), 4.45-4.10 (m, 2H), 3.76 (d, J=13.4 Hz, 1H), 3.63 (s, 3H), 3.57-3.39 (m, 2H), 1.49 (s, 9H), 1.26 (d, J=6.7 Hz, 3H), 1.18 (d, J=6.1 Hz, 3H) ppm. MS: M/e 409 (M+1)⁺.

### Step I: tert-butyl (2R,5S)-4-(2-(chloromethyl)-4-methyl-5-oxo-4,5-dihydrothiazolo[5,4-b]pyridin-7-yl)-2,5-dimethylpiperazine-1-carboxylate

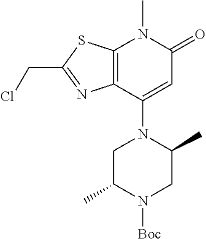 [SMILES: C[C@@H]1CN(c2cc(=O)n(C)c3sc(CCl)nc23)[C@@H](C)CN1C(=O)OC(C)(C)C]

To a mixture of tert-butyl (2R,5S)-4-(2-(hydroxymethyl)-4-methyl-5-oxo-4,5-dihydrothiazolo[5,4-b]pyridin-7-yl)-2,5-dimethylpiperazine-1-carboxylate (370 mg, 0.9 mmol) in DCM (5 mL) was added SOCl₂ (322 mg, 2.7 mmol). The resulting mixture was stirred at rt for 30 minutes. The reaction was diluted with NaHCO₃ (aq.), extracted with DCM, washed with brine, dried over Na₂SO₄, filtered, and concentrated. The resulting residue was used in the next step (400 mg, crude). MS: M/e 427 (M+1)⁺.

### Step G: tert-butyl (2R,5S)-4-(2-(cyanomethyl)-4-methyl-5-oxo-4,5-dihydrothiazolo[5,4-b]pyridin-7-yl)-2,5-dimethylpiperazine-1-carboxylate

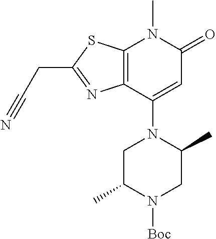 [SMILES: C[C@@H]1CN(c2cc(=O)n(C)c3sc(CC#N)nc23)[C@@H](C)CN1C(=O)OC(C)(C)C]

To a mixture of tert-butyl (2R,5S)-4-(2-(chloromethyl)-4-methyl-5-oxo-4,5-dihydrothiazolo[5,4-b]pyridin-7-yl)-2,5-dimethylpiperazine-1-carboxylate (400 mg, crude) in DCM (4 mL) was added TMSCN (278 mg, 2.81 mmol), TBAF (1M in THF, 2.8 ml, 2.8 mmol). The resulting mixture was stirred at rt for 2 hours. The reaction was diluted with water, extracted with DCM, washed with brine, dried over Na₂SO₄, filtered, and concentrated. The residue was purified by Combi-flash (EtOAc:PE=0-100% in 20 minutes) to give the titled compound (130 mg, 33%). MS: M/e 418 (M+1)⁺.

### Step K: 2-(7-((2S,5R)-2,5-dimethylpiperazin-1-yl)-4-methyl-5-oxo-4,5-dihydrothiazolo[5,4-b]pyridin-2-yl)acetonitrile

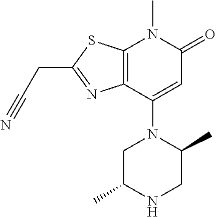 [SMILES: C[C@@H]1CN(c2cc(=O)n(C)c3sc(CC#N)nc23)[C@@H](C)CN1]

To tert-butyl (2R,5S)-4-(2-(cyanomethyl)-4-methyl-5-oxo-4,5-dihydrothiazolo[5,4-b]pyridin-7-yl)-2,5-dimethylpiperazine-1-carboxylate (130 mg) in DCM (4 mL) was added TFA (1.5 ml). The mixture was stirred at rt for 4 hours. The mixture was concentrated to dryness and diluted with water. The mixture was extracted with EtOAc. The aqueous phase was adjusted pH to 12-13 with saturated sodium carbonate solution and extracted with DCM. The combined organic layers were washed with brine, dried over Na₂SO₄, and concentrated. The residue 80 mg (yield=81%) was used in the next step. MS: M/e 318 (M+1)⁺.

### Step L: 2-(7-((2S,5R)-2,5-dimethyl-4-(1-(quinoxalin-6-yl)ethyl)piperazin-1-yl)-4-methyl-5-oxo-4,5-dihydrothiazolo[5,4-b]pyridin-2-yl)acetonitrile

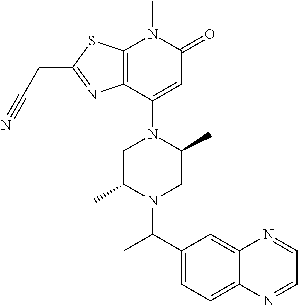 [SMILES: CC(c1ccc2nccnc2c1)N1C[C@H](C)N(c2cc(=O)n(C)c3sc(CC#N)nc23)C[C@H]1C]

To the 2-(7-((2S,5R)-2,5-dimethylpiperazin-1-yl)-4-methyl-5-oxo-4,5-dihydrothiazolo[5,4-b]pyridin-2-yl)acetonitrile (80 mg, 0.25 mmol) in MeCN (3 mL) was added 1-(quinoxalin-6-yl)ethan-1-ol (66 mg, 0.38 mmol), (cyanomethyl)trimethylphosphonium iodide (121 mg, 0.5 mmol) and DIPEA (129 mg, 1 mmol). The resulted mixture was stirred at 90° C. for overnight. The mixture was concentrated to dryness. The reaction was quenched with water, extracted with EtOAc, washed with brine, dried over Na₂SO₄, filtered, and concentrated. The resulting residue was purified by flash column chromatography (MeOH:DCM=0-10% in 20 minutes) to give the titled compound, which was further separated into Compound A194a (12 mg) and Compound A194b (11 mg) by chiral prep-HPLC. The chiral separation conditions are shown below.

| | |
|---|---|
| Column | CHIRALPAK IH |
| Column Size | 2 cm × 25 cm, 5 um |
| Mobile Phase A | MtBE (0.5% 2 mM NH3—MeOH) |
| Mobile Phase B | MeOH |
| Flow Rate | 20 mL/min |
| Wave Length | UV 220 nm |
| Temperature | 25° C. |
| Prep-HPLC Equipment | Prep-HPLC-Gilson |

Compound A194a (the earlier peak): ¹H NMR (400 MHz, CD₃OD) δ 8.87 (d, J=3.9 Hz, 2H), 8.14-8.02 (m, 3H), 5.71 (s, 1H), 4.39 (s, 1H), 3.98 (d, J=6.6 Hz, 1H), 3.60 (s, 3H), 3.49 (d, J=3.4 Hz, 1H), 3.33 (s, 1H), 3.14-3.05 (m, 1H), 2.93 (d, J=12.5 Hz, 4H), 1.45 (dd, J=11.7, 6.5 Hz, 6H), 1.09 (d, J=6.5 Hz, 3H) ppm. MS: M/e 474 (M+1)⁺.

Compound A194b (the later peak): ¹H NMR (400 MHz, CD₃OD) δ 8.87 (d, J=4.2 Hz, 2H), 8.06 (q, J=8.8 Hz, 3H), 5.71 (s, 1H), 4.68-4.35 (m, 2H), 3.83 (d, J=6.1 Hz, 1H), 3.69 (d, J=10.4 Hz, 2H), 3.60 (s, 3H), 3.32 (s, 1H), 2.87 (d, J=8.5 Hz, 2H), 2.20 (d, J=11.7 Hz, 1H), 1.47 (d, J=6.5 Hz, 3H), 1.26 (t, J=9.4 Hz, 6H) ppm. MS: M/e 474 (M+1)⁺.

### Compound A195: 2-(7-((2S,5R)-4-(1-(4-fluoro-2-(trifluoromethyl)phenyl)ethyl)-2,5-dimethylpiperazin-1-yl)-4-methyl-5-oxo-4,5-dihydrothiazolo[5,4-b]pyridin-2-yl)acetonitrile

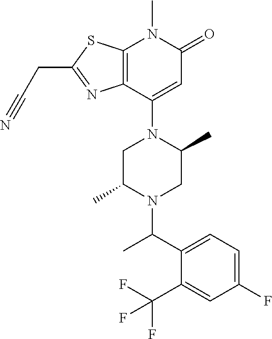 [SMILES: CC(c1ccc(F)cc1C(F)(F)F)N1C[C@H](C)N(c2cc(=O)n(C)c3sc(CC#N)nc23)C[C@H]1C]

### Step 1: tert-butyl (2S,5R)-4-(1-(4-fluoro-2-(trifluoromethyl)phenyl)ethyl)-2,5-dimethylpiperazine-1-carboxylate

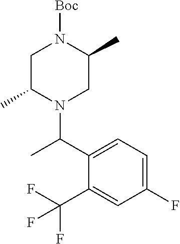 [SMILES: CC(c1ccc(F)cc1C(F)(F)F)N1C[C@H](C)N(C(=O)OC(C)(C)C)C[C@H]1C]

A sealed tube was charged with 1-(4-fluoro-2-(trifluoromethyl)phenyl)ethan-1-ol (1.56 g, 7.5 mmol), tert-butyl (2S,5R)-2,5-dimethylpiperazine-1-carboxylate (1.07 g, 5 mmol), (cyanomethyl)trimethylphosphonium iodide (2.4 g, 10 mmol), DIPEA (2.58 g, 20 mmol) and acetonitrile (15 ml). The mixture was stirred at 100° C. overnight, cooled to rt. The reaction was purified by flash column chromatography (EtOAc:PE=0-60% in 25 minutes) to give the titled compound (2.5 g, crude). MS: M/e 405 (M+1)⁺.

### Step 2: (2R,5S)-1-(1-(4-fluoro-2-(trifluoromethyl)phenyl)ethyl)-2,5-dimethylpiperazine

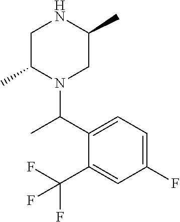 [SMILES: CC(c1ccc(F)cc1C(F)(F)F)N1C[C@H](C)NC[C@H]1C]

To tert-butyl (2S,5R)-4-(1-(4-fluoro-2-(trifluoromethyl)phenyl)ethyl)-2,5-dimethylpiperazine-1-carboxylate (2.5 g) in DCM (20 mL) was added TFA (7 ml). The mixture was stirred at rt for 4 hours. The mixture was concentrated to dryness and diluted with water. The mixture was extracted with EtOAc. The aqueous phase was adjusted pH to 12-13 with saturated sodium carbonate solution and extracted with DCM. The combined organic layers were washed with brine, dried over Na₂SO₄, and concentrated to give the titled compound (1.1 g, 59%). MS: M/e 305 (M+1)⁺.

### Step 3: 2-bromo-7-((2S,5R)-4-(1-(4-fluoro-2-(trifluoromethyl)phenyl)ethyl)-2,5-dimethylpiperazin-1-yl)-4-methylthiazolo[5,4-b]pyridin-5 (4H)-one

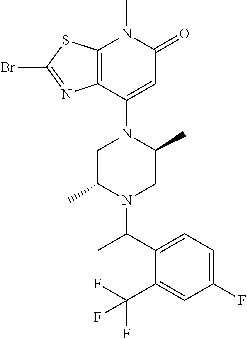 [SMILES: CC(c1ccc(F)cc1C(F)(F)F)N1C[C@H](C)N(c2cc(=O)n(C)c3sc(Br)nc23)C[C@H]1C]

To tert-butyl (2S,5R)-4-(1-(4-fluoro-2-(trifluoromethyl)phenyl)ethyl)-2,5-dimethylpiperazine-1-carboxylate (85 mg, crude) in MeCN (3 mL) was added 2-bromo-4-methyl-5-oxo-4,5-dihydrothiazolo[5,4-b]pyridin-7-yl trifluoromethanesulfonate (100 mg, 0.25 mmol) and DIPEA (129 mg, 1 mmol). The resulted mixture was stirred at 90° C. for overnight. The mixture was concentrated to dryness. The reaction was quenched with water, extracted with EtOAc, washed with brine, dried over Na₂SO₄, filtered, and concentrated. The resulting residue was purified by flash column chromatography (EtOAc:PE=0-60% in 20 minutes) to give the titled compound (100 mg, 72%). MS: M/e 547 (M+1)⁺.

### Step 4: methyl 7-((2S,5R)-4-(1-(4-fluoro-2-(trifluoromethyl)phenyl)ethyl)-2,5-dimethylpiperazin-1-yl)-4-methyl-5-oxo-4,5-dihydrothiazolo[5,4-b]pyridine-2-carboxylate

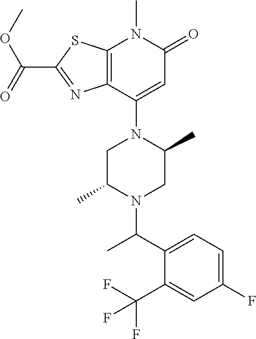 [SMILES: COC(=O)c1nc2c(N3C[C@@H](C)N(C(C)c4ccc(F)cc4C(F)(F)F)C[C@@H]3C)cc(=O)n(C)c2s1]

To a mixture of 2-bromo-7-((2S,5R)-4-(1-(4-fluoro-2-(trifluoromethyl)phenyl)ethyl)-2,5-dimethylpiperazin-1-yl)-4-methylthiazolo[5,4-b]pyridin-5 (4H)-one (100 mg, 0.18 mmol) was dissolved in MeOH (5 ml) added Pd(dppf)Cl₂ (30 mg, 0.01 mmol), Et₃N (36 mg, 0.36 mmol). The reaction was stirred at 90° C. for overnight under carbon monoxide (20 atm) atmosphere. The reaction was quenched with water and extracted with EtOAc. The combined organic layers were washed with brine, dried over Na₂SO₄, and concentrated. The residue was purified by flash column chromatography (EtOAc:PE=0-100% in 20 minutes) to give the titled compound (70 mg, 73%). MS: M/e 527 (M+1)⁺.

### Step 5: 7-((2S,5R)-4-(1-(4-fluoro-2-(trifluoromethyl)phenyl)ethyl)-2,5-dimethylpiperazin-1-yl)-2-(hydroxymethyl)-4-methylthiazolo[5,4-b]pyridin-5 (4H)-one

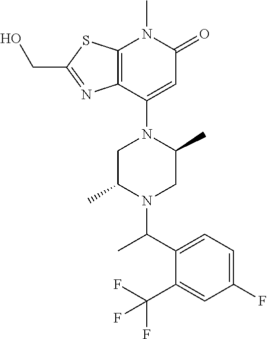 [SMILES: CC(c1ccc(F)cc1C(F)(F)F)N1C[C@H](C)N(c2cc(=O)n(C)c3sc(CO)nc23)C[C@H]1C]

To a mixture of methyl 7-((2S,5R)-4-(1-(4-fluoro-2-(trifluoromethyl)phenyl)ethyl)-2,5-dimethylpiperazin-1-yl)-4-methyl-5-oxo-4,5-dihydrothiazolo[5,4-b]pyridine-2-carboxylate (70 mg, 0.13 mmol) in THF (4 mL) was added NaBH₄ (20 mg, 0.53 mmol). The resulting mixture was stirred at 70° C. for overnight. The reaction was diluted with water, extracted with EtOAc washed with brine, dried over Na₂SO₄, filtered, and concentrated to dryness. The resulting residue was used in the next step (80 mg, crude). MS: M/e 499 (M+1)⁺.

### Step 6: 2-(chloromethyl)-7-((2S,5R)-4-(1-(4-fluoro-2-(trifluoromethyl)phenyl)ethyl)-2,5-dimethylpiperazin-1-yl)-4-methylthiazolo[5,4-b]pyridin-5 (4H)-one

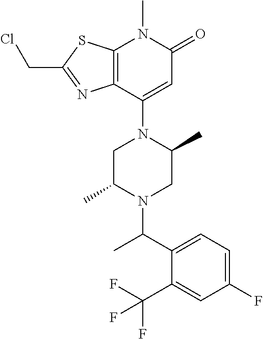 [SMILES: CC(c1ccc(F)cc1C(F)(F)F)N1C[C@H](C)N(c2cc(=O)n(C)c3sc(CCl)nc23)C[C@H]1C]

To a mixture of 7-((2S,5R)-4-(1-(4-fluoro-2-(trifluoromethyl)phenyl)ethyl)-2,5-dimethylpiperazin-1-yl)-2-(hydroxymethyl)-4-methylthiazolo[5,4-b]pyridin-5 (4H)-one (80 mg, crude) in DCM (4 mL) was added SOCl₂ (57 mg, 0.48 mmol). The resulting mixture was stirred at 0° C. for 30 minutes. The reaction was diluted with NaHCO₃ (aq.), extracted with DCM, washed with brine, dried over Na₂SO₄, filtered, and concentrated to dryness. The resulting residue was used in the next step (60 mg, crude). MS: M/e 517 (M+1)⁺.

### Step 7: 2-(7-((2S,5R)-4-(1-(4-fluoro-2-(trifluoromethyl)phenyl)ethyl)-2,5-dimethylpiperazin-1-yl)-4-methyl-5-oxo-4,5-dihydrothiazolo[5,4-b]pyridin-2-yl)acetonitrile

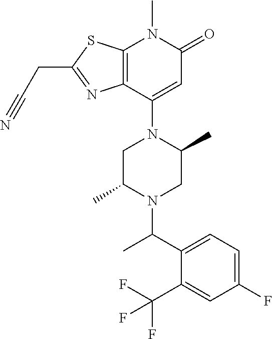 [SMILES: CC(c1ccc(F)cc1C(F)(F)F)N1C[C@H](C)N(c2cc(=O)n(C)c3sc(CC#N)nc23)C[C@H]1C]

To a mixture of 2-(chloromethyl)-7-((2S,5R)-4-(1-(4-fluoro-2-(trifluoromethyl)phenyl)ethyl)-2,5-dimethylpiperazin-1-yl)-4-methylthiazolo[5,4-b]pyridin-5 (4H)-one (60 mg, crude) in DCM (4 mL) was added TMSCN (23 mg, 0.23 mmol), TBAF (1M in THF, 0.23 ml, 0.23 mmol). The resulting mixture was stirred at rt for 2 hours. The reaction was diluted with water, extracted with DCM, washed with brine, dried over Na₂SO₄, filtered, and concentrated to dryness. The resulting residue was purified by Prep-TLC (MeOH:DCM=1:13) to give the titled compound (13 mg). ¹H NMR (400 MHz, CD₃OD) δ 8.14-8.01 (m, 1H), 7.40 (t, J=9.0 Hz, 2H), 5.70 (s, 1H), 4.40 (s, 1H), 4.01 (d, J=57.5 Hz, 1H), 3.65 (s, 1H), 3.61 (d, J=7.5 Hz, 3H), 3.48-3.33 (m, 1H), 2.99 (dd, J=42.5, 9.4 Hz, 1H), 2.82 (d, J=8.4 Hz, 2H), 2.00 (d, J=12.1 Hz, 1H), 1.48-1.30 (m, 4H), 1.23 (dd, J=28.7, 6.5 Hz, 5H), 1.04 (d, J=6.4 Hz, 1H) ppm. MS: M/e 508 (M+1)⁺.

The following compounds were prepared according to the similar procedures under appropriate conditions:

| | | | | Procedures | |
|---|---|---|---|---|---|
| | | | | similar | |
| | | | | to or | |
| Compound | Name | Structure | Yield | following | Characterization |
| A2 | 2-(7-((2S,5R)-4-(1-(4-fluoro-2- (trifluoromethyl)phenyl)ethyl)- 2,5-dimethylpiperazin-1-yl)-3,4- dimethyl-5-oxo-4,5-dihydro-3H- imidazo[4,5-b]pyridin-2- yl)acetonitrile | 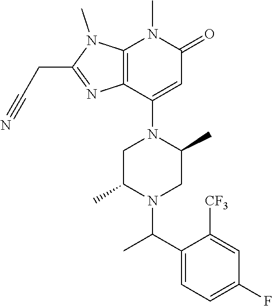 [SMILES: CC(c1ccc(F)cc1C(F)(F)F)N1C[C@H](C)N(c2cc(=O)n(C)c3c2nc(CC#N)n3C)C[C@H]1C] | 0.4 mg | Compound A1 | ¹H NMR (400 MHz, CD₃OD) δ 8.15-8.00 (m, 1H), 7.48-7.30 (m, 2H), 5.51 (s, 1H), 4.21 (s, 2H), 3.97 (s, 3H), 3.83 (s, 3H), 3.70- 3.53 (m, 1H), 3.50-3.10 (m, 2H), 2.80- 2.65 (m, 3H), 2.10-1.95 (m, 1H), 1.64- 1.20 (m, 6H), 1.19-1.01 (m, 3H) ppm. MS: M/e 505 (M + 1)⁺. |
| A3 | 2-(7-((2S,5R)-4-(1-(4- fluorophenyl)ethyl)-2,5- dimethylpiperazin-1-yl)-3,4- dimethyl-5-oxo-4,5-dihydro-3H- imidazo[4,5-b]pyridin-2- yl)acetonitrile | 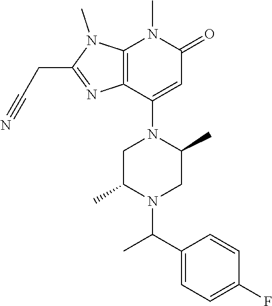 [SMILES: CC(c1ccc(F)cc1)N1C[C@H](C)N(c2cc(=O)n(C)c3c2nc(CC#N)n3C)C[C@H]1C] | 0.5 mg | Compound A1 | ¹H NMR (400 MHz, CD₃OD) δ 7.60-7.45 (m, 2H), 7.25-7.10 (m, 2H), 5.52 (s, 1H), 4.23 (s, 2H), 3.98 (s, 3H), 3.84 (s, 3H), 3.75- 3.53 (m, 2H), 2.85-2.65 (m, 2H), 2.10- 1.98 (m, 2H), 1.44-1.20 (m, 9H) ppm. MS: M/e 437 (M + 1)⁺. |
| A8 | 2-(7-((2S,5R)-2,5-dimethyl-4-(1- (4-methyl-2- (trifluoromethyl)phenyl)ethyl) piperazin-1-yl)-3,4-dimethyl-5- oxo-4,5-dihydro-3H- imidazo[4,5-b]pyridin-2- yl)acetonitrile | 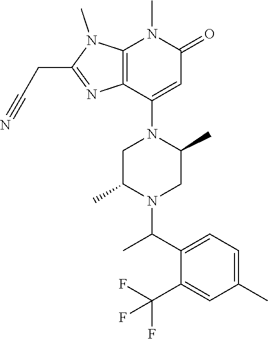 [SMILES: Cc1ccc(C(C)N2C[C@H](C)N(c3cc(=O)n(C)c4c3nc(CC#N)n4C)C[C@H]2C)c(C(F)(F)F)c1] | A8a (0.6 mg) A8b (0.5 mg) | Compound A1 | Compound A8a (the earlier peak): ¹H NMR (400 MHz, CD₃OD) δ 7.87 (d, J = 8.6 Hz, 1H), 7.47-7.41 (m, 2H), 5.51 (s, 1H), 4.26- 4.17 (m, 1H), 3.97 (s, 3H), 3.95-3.85 (m, 1H), 3.83 (s, 3H), 3.70-3.51 (m, 2H), 2.85- 3.55 (m, 4H), 2.39 (s, 3H), 2.01 (d, J = 11.9 Hz, 1H), 1.30 (d, J = 6.4 Hz, 3H), 1.18- 1.11 (m, 6H) ppm. MS: M/e 501 (M + 1)⁺. Compound A8b (the later peak): ¹H NMR (400 MHz, CD₃OD) δ 7.92 (d, J = 8.4 Hz, 1H), 7.46 (s, 2H), 5.51 (s, 1H), 4.23-4.16 (m, 1H), 4.05-3.99 (m, 1H), 3.97 (s, 3H), 3.83 (s, 3H), 3.37-3.33 (m, 2H), 3.04- 2.80 (m, 2H), 2.79-2.60 (m, 3H), 2.40 (s, 3H), 1.42 (d, J = 6.7 Hz, 3H), 1.24 (d, J = 6.4 Hz, 3H), 0.98 (d, J = 6.4 Hz, 3H) ppm. MS: M/e 501 (M + 1)⁺. |
| A15 | 2-(7-((2S,5R)-2,5-dimethyl-4-(1- (4-methyl-2- (trifluoromethyl)phenyl)ethyl) piperazin-1-yl)-6-fluoro-3,4- dimethyl-5-oxo-4,5-dihydro-3H- imidazo[4,5-b]pyridin-2- yl)acetonitrile | 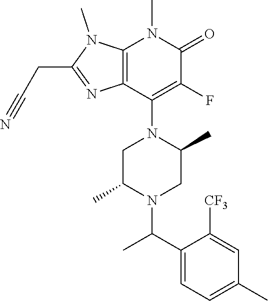 [SMILES: Cc1ccc(C(C)N2C[C@H](C)N(c3c(F)c(=O)n(C)c4c3nc(CC#N)n4C)C[C@H]2C)c(C(F)(F)F)c1] | A15a (4 mg) A15b (5 mg) | Compound A14 | Compound A15a (the earlier peak): ¹H NMR (400 MHz, CD₃OD) δ 7.88 (d, J = 8.5 Hz, 1H), 7.43 (s, 2H), 4.64 (br, 1H), 4.26- 4.10 (m, 2H), 4.03-3.95 (m, 4H), 3.94- 3.85 (m, 4H), 3.56 (br, 1H), 2.88 (d, J = 8.3 Hz, 1H), 2.83-2.67 (m, 1H), 2.39 (s, 3H), 1.99 (d, J = 11.6 Hz, 1H), 1.30 (d, J = 6.4 Hz, 3H), 1.25 (d, J = 6.6 Hz, 3H), 1.14 (d, J = 6.5 Hz, 3H) ppm. MS: M/e 519 (M + 1)⁺. Compound A15b (the later peak): ¹H NMR (400 MHz, CD₃OD) δ 7.94 (d, J = 8.1 Hz, 1H), 7.49-7.43 (m, 2H), 4.95 (br, 1H), 4.05 (s, 1H), 3.98 (s, 3H), 3.89 (s, 3H), 3.79 (d, J = 7.4 Hz, 2H), 3.11 (d, J = 11.1 Hz, 1H), 2.88 (d, J = 12.0 Hz, 1H), 2.86-2.70 (m, 3H), 2.40 (s, 3H), 1.51 (d, J = 6.7 Hz, 3H), 1.23 (d, J = 6.4 Hz, 3H), 0.98 (d, J = 6.5 Hz, 3H) ppm. MS: M/e 519 (M + 1)⁺. |
| A17 | 2-(7-((2S,5R)-2,5-dimethyl-4-(1- (3-methyl-2- (trifluoromethyl)phenyl)ethyl) piperazin-1-yl)-6-fluoro-3,4- dimethyl-5-oxo-4,5-dihydro-3H- imidazo[4,5-b]pyridin-2- yl)acetonitrile | 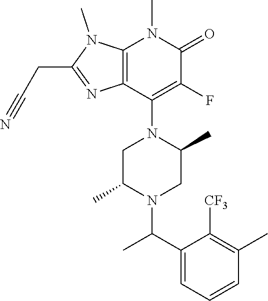 [SMILES: Cc1cccc(C(C)N2C[C@H](C)N(c3c(F)c(=O)n(C)c4c3nc(CC#N)n4C)C[C@H]2C)c1C(F)(F)F] | A17a (4 mg) A17b (5 mg) | Compound A14 | Compound A17a (the earlier peak): ¹H NMR (400 MHz, DMSO-d₆) δ 7.64 (d, J = 7.9 Hz, 1H), 7.41 (d, J = 12.7 Hz, 2H), 4.63 (s, 1H), 4.38 (s, 2H), 3.88 (s, 3H), 3.76 (s, 3H), 3.74 (s, 1H), 3.56 (dd, J = 44.2, 12.3 Hz, 2H), 2.97 (d, J = 7.8 Hz, 1H), 2.61 (d, J = 11.0 Hz, 2H), 2.46 (s, 3H), 1.31 (d, J = 6.5 Hz, 3H), 1.26 (d, J = 6.5 Hz, 3H), 0.94 (d, J = 6.4 Hz, 3H) ppm. MS: M/e 519 (M + 1)⁺. Compound A17b (the later peak): ¹H NMR (400 MHz, DMSO-d₆) δ 7.62 (d, J = 8.2 Hz, 1H), 7.50-7.35 (m, 2H), 4.41 (s, 1H), 4.37 (s, 2H), 3.88 (s, 3H), 3.82 (d, J = 14.3 Hz, 2H), 3.76 (s, 3H), 3.62 (d, J = 6.2 Hz, 1H), 3.36 (s, 1H), 2.70 (d, J = 7.7 Hz, 1H), 2.44 (s, 3H), 2.02 (d, J = 8.8 Hz, 1H), 1.26 (d, J = 6.3 Hz, 3H), 1.16 (d, J= 6.4 Hz, 3H), 1.07 (d, J = 6.4 Hz, 3H) ppm. MS: M/e 519 |
| | | | | | (M + 1)⁺. |
| A32 | 2-(6-((2S,5R)-2,5-dimethyl-4-(1- (4-methyl-2- (trifluoromethyl)phenyl)ethyl) piperazin-1-yl)-3,9-dimethyl-2- oxo-3,9-dihydro-2H-purin-8- yl)acetonitrile | 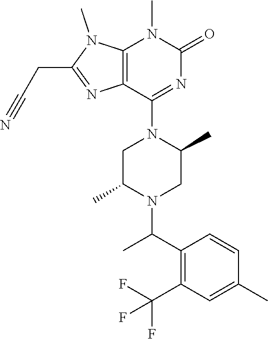 [SMILES: Cc1ccc(C(C)N2C[C@H](C)N(c3nc(=O)n(C)c4c3nc(CC#N)n4C)C[C@H]2C)c(C(F)(F)F)c1] | A32a (3 mg) A32b (3 mg) | Compound A24 | Compound A32a (the earlier peak): ¹H NMR (400 MHz, CD₃OD) δ 7.88 (d, J = 8.5 Hz, 1H), 7.44 (d, J = 6.0 Hz, 2H), 5.93-5.87 (m, 1H), 5.03 (s, 0.5H), 4.84-4.79 (m, 0.5H), 4.20 (d, J = 19.0 Hz, 1H), 3.93 (s, 3H), 3.89-3.83 (m, 1H), 3.81-3.73 (m, 4H), 3.68-3.62 (m, 1H), 3.46-3.42 (m, 0.5H), 2.81-2.62 (m, 1.5H), 2.39 (s, 3H), 2.01 (d, J = 12.2 Hz, 1H), 1.30 (d, J = 6.3 Hz, 3H), 1.22 (dd, J= 16.3, 6.5 Hz, 3H), 1.04 (d, J = 6.1 Hz, 3H) ppm. MS: M/e 502 (M + 1)⁺. Compound A32b (the later peak): ¹H NMR (400 MHz, CD₃OD) δ 7.92 (d, J = 8.5 Hz, 1H), 7.46 (d, J = 6.2 Hz, 2H), 6.18 (s, 0.5H), 5.62 (d, J = 13.2 Hz, 0.5H), 5.27 (s, 0.5H), 4.57 (d, J= 13.4 Hz, 0.5H), 4.20 (d, J = 29.2 Hz, 1H), 4.04-3.94 (m, 1H), 3.93 (d, J = 14.9 Hz, 3H), 3.78 (s, 3H), 3.56-3.51 (m, 0.5H), 3.22-3.17 (m, 0.5H), 2.94 (s, 1H), 2.91-2.85 (m, 1H), 2.84-2.60 (m, 2H), 2.40 (s, 3H), 1.50 (dd, J = 26.2, 6.6 Hz, 3H), |
| | | | | | 1.27-1.18 (m, 3H), 0.94-0.81 (m, 3H) ppm. |
| | | | | | MS: M/e 502(M + 1)⁺ |
| A38 | 2-(6-((2S,5R)-4-(1-(3- (difluoromethyl)-5- fluorophenyl)ethyl)-2,5-dimethyl- piperazin-1-yl)-9-ethyl-3- methyl-2-oxo-3,9-dihydro-2H- purin-8-yl)acetonitrile | 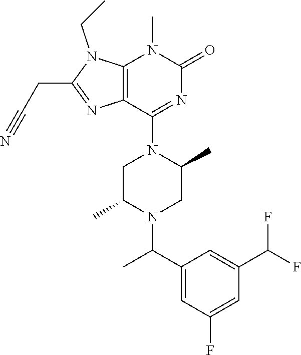 [SMILES: CCn1c(CC#N)nc2c(N3C[C@@H](C)N(C(C)c4cc(F)cc(C(F)F)c4)C[C@@H]3C)nc(=O)n(C)c21] | 42 mg | Compound A35 | ¹H NMR (400 MHz, CD₃OD) δ 7.43 (d, J = 8.4 Hz, 1H), 7.32 (s, 1H), 7.17 (t, J = 9.6 Hz, 1H), 6.78 (td, J = 56.2, 10.4 Hz, 1H), 6.50-4.50 (m, 2H), 4.37 (d, J = 6.9 Hz, 2H), 4.23 (s, 1H), 3.76 (s, 3H), 3.73-3.51 (m, 2H), 3.29-2.10 (m, 4H), 1.49-1.25 (m, 9H), 1.06-0.89 (m, 3H) ppm. MS: M/e 502 (M + 1)⁺ |
| A39 | 2-(6-((2S,5R)-4-(1-(3-(1,1- difluoroethyl)-4- methoxyphenyl)ethyl)-2,5- dimethylpiperazin-1-yl)-9-ethyl- 3-methyl-2-oxo-3,9-dihydro-2H- purin-8-yl)acetonitrile | 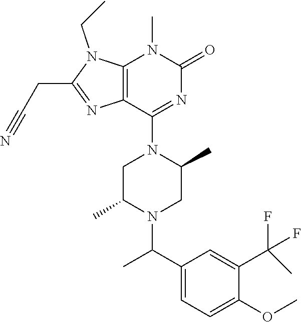 [SMILES: CCn1c(CC#N)nc2c(N3C[C@@H](C)N(C(C)c4ccc(OC)c(C(C)(F)F)c4)C[C@@H]3C)nc(=O)n(C)c21] | 22 mg, 14% | Compound A35 | ¹H NMR (400 MHz, DMSO-d6) δ 7.60- 7.44 (m, 2H), 7.21-7.12 (m, 1H), 5.96- 4.54 (m, 2H), 4.47 (s, 2H), 4.32 (s, 2H), 3.90 (d, J = 3.6 Hz, 3H), 3.74-3.48 (m, 5H), 2.89-2.60 (m, 2H), 2.39-2.17 (m, 1H), 2.03 (td, J = 19.0, 7.2 Hz, 3H), 1.45- 1.20 (m, 9H), 1.03-0.86 (m, 3H). ppm. MS: M/e 528 (M + 1)⁺. |
| A40 | 2-(6-((2S,5R)-2,5-dimethyl-4-(1- (4- (trifluoromethyl)phenyl)ethyl)pi- perazin-1-yl)-9-ethyl-3- methyl- 2-oxo-3,9-dihydro-2H-purin-8- yl)acetonitrile | 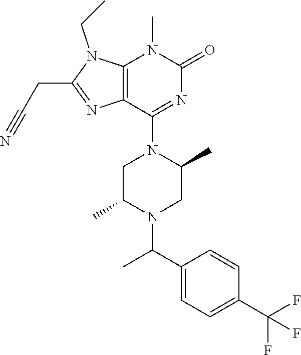 [SMILES: CCn1c(CC#N)nc2c(N3C[C@@H](C)N(C(C)c4ccc(C(F)(F)F)cc4)C[C@@H]3C)nc(=O)n(C)c21] | 15 mg, 16% | Compound A35 | ¹H NMR (400 MHz, MeOD) δ 7.66-7.61 (m, 4H), 5.97-5.91 (m, 1H), 5.31-5.24 (m, 1H), 4.38-4.36 (m, 2H), 4.23 (s, 2H), 3.76 (s, 3H), 3.75-3.70 (m, 1H), 3.65- 3.62 (m, 1H), 3.24-3.22 (m, 1H), 2.92- 2.82 (m, 2H), 1.49-1.44 (m, 3H), 1.38- 1.29 (m, 5H), 1.08-1.06 (m, 2H), 0.94- 0.90 (m, 2H) ppm. MS: M/e 502 (M + 1)⁺. |
| A45 | 2-(6-((2S,5R)-2,5-dimethyl-4-(1- (m-tolyl)ethyl)piperazin-1-yl)-9- ethyl-3-methyl-2-oxo-3,9- dihydro-2H-purin-8- yl)acetonitrile | 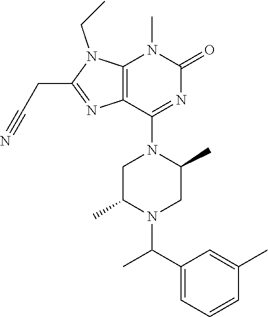 [SMILES: CCn1c(CC#N)nc2c(N3C[C@@H](C)N(C(C)c4cccc(C)c4)C[C@@H]3C)nc(=O)n(C)c21] | 30 mg | Compound A35 | ¹H NMR (400 MHz, CD₃OD) δ 7.23- 7.11 (m, 3H), 7.10-7.01 (m, 1H), 6.01- 4.95 (m, 2H), 4.63-4.09 (m, 3H), 3.76 (s, 3H), 3.75-3.69 (m, 1H), 3.68-3.35 (m, 2H), 2.98-2.76 (m, 2H), 2.75-2.10 (m, 1H), 2.33 (d, J = 7.9 Hz, 3H), 1.46 (t, J = 7.1 Hz, 3H), 1.37 (d, J = 6.3 Hz, 6H), 0.96 (dd, J = 62.1, 5.9 Hz, 3H) ppm. MS: M/e 448 (M + 1)⁺. |
| A46 | 2-(9-ethyl-6-((2S,5R)-4-(1-(3- methoxyphenyl)ethyl)-2,5- dimethylpiperazin-1-yl)-3- methyl-2-oxo-3,9-dihydro-2H- purin-8-yl)acetonitrile | 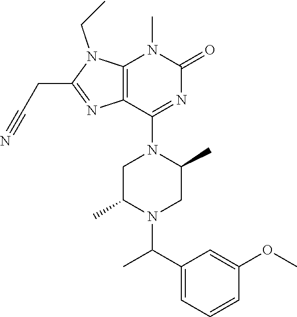 [SMILES: CCn1c(CC#N)nc2c(N3C[C@@H](C)N(C(C)c4cccc(OC)c4)C[C@@H]3C)nc(=O)n(C)c21] | 53 mg | Compound A35 | ¹H NMR (400 MHz, CD₃OD) δ 7.31- 7.11 (m, 1H), 6.71-6.89 (m, 2H), 6.86- 6.69 (m, 1H), 6.02-4.99 (m, 2H), 4.69- 4.04 (m, 3H), 3.86-3.67 (m, 7H), 3.65- 3.36 (m, 2H), 3.22-2.61 (m, 2H), 2.60- 2.10 (m,1H), 1.46 (t, J = 6.9 Hz, 3H), 1.36 (dd, J = 11.7, 5.6 Hz, 6H), 0.96 (dd, J = 58.3, 6.0 Hz, 3H) ppm. MS: M/e 464 (M + 1)⁺. |
| A47 | 2-(9-ethyl-6-((2S,5R)-4-(1-(2- methoxypyridin-4-yl)ethyl)-2,5- dimethyl piperazin-1-yl)-3- methyl-2-oxo-3,9-dihydro-2H- purin-8-yl)acetonitrile | 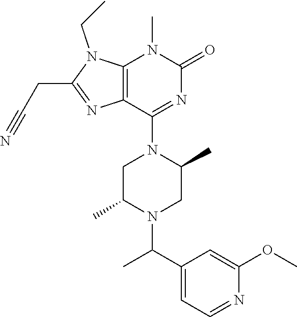 [SMILES: CCn1c(CC#N)nc2c(N3C[C@@H](C)N(C(C)c4ccnc(OC)c4)C[C@@H]3C)nc(=O)n(C)c21] | 50 mg, 71% | Compound A35 | ¹H NMR (400 MHz, CD₃OD) δ 8.06 (dd, J = 10.9, 5.4 Hz, 1H), 7.04 (t, J = 5.3 Hz, 1H), 6.84 (d, J = 6.7 Hz, 1H), 6.46-4.88 (m, 2H), 4.42-4.32 (m, 2H), 4.22 (s, 1H), 3.90 (d, J = 4.5 Hz, 3H), 3.76 (s, 3H), 3.65- 3.45 (m, 1H), 3.37-3.32 (m, 2H), 2.93- 2.00 (m, 3H), 1.57-1.32 (m, 6H), 1.31- 1.19 (m, 3H), 1.06-0.89 (m, 3H) ppm. MS: M/e 465 (M + 1)⁺ |
| A73 | 2-(6-((2S,5R)-4-(1-(7-chloro- 3,3-dimethyl-2,3- dihydrobenzo[b][1,4]dioxin-6- yl)ethyl)-2,5-dimethylpiperazin- 1-yl)-9-ethyl-3-methyl-2-oxo- 3,9-dihydro-2H-purin-8- yl)acetonitrile | 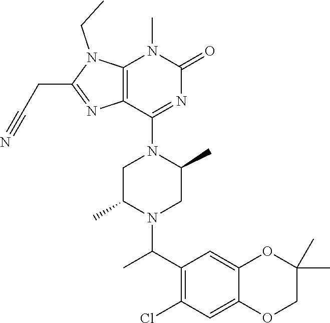 [SMILES: CCn1c(CC#N)nc2c(N3C[C@@H](C)N(C(C)c4cc5c(cc4Cl)OCC(C)(C)O5)C[C@@H]3C)nc(=O)n(C)c21] | 35 mg, 21% | Compound A35 | ¹H NMR (400 MHz, CD₃OD) δ 7.11 (d, J = 25.4 Hz, 1H), 6.85 (d, J = 10.4 Hz, 1H), 6.21-5.62 (m, 1H), 5.15 (d, J = 70.0 Hz, 0.5H), 4.73-4.61 (m, 0.5H), 4.37 (q, J = 7.0 Hz, 2H), 4.23 (s, 2H), 4.07 (d, J = 6.3 Hz, 0.5H), 3.98-3.87 (m, 2.5H), 3.76 (s, 3H), 3.62-3.43 (m, 1H), 3.13-2.68 (m, 2.5H), 2.21 (d, J = 12.1 Hz, 0.5H), 1.46 (t, J = 7.1 Hz, 4.5H), 1.33 (d, J = 11.3 Hz, 6.5H), 1.25 (dd, J = 20.3, 6.3 Hz, 4H), 0.99 (dd, J = 52.6, 5.2 Hz, 3H) ppm. MS: M/e 554 (M + 1)⁺ |
| A75 | 2-(6-((2S,5R)-4-(1-(2,2- dimethyl-2,3-dihydro- [1,4]dioxino[2,3-c]pyridin-7- yl)ethyl)-2,5-dimethylpiperazin- 1-yl)-9-ethyl-3-methyl-2-oxo- 3,9-dihydro-2H-purin-8- yl)acetonitrile | 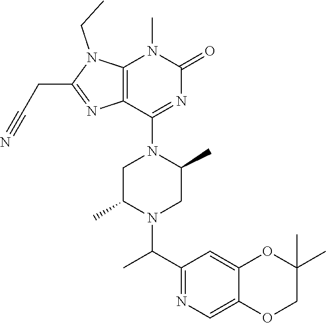 [SMILES: CCn1c(CC#N)nc2c(N3C[C@@H](C)N(C(C)c4cc5c(cn4)OCC(C)(C)O5)C[C@@H]3C)nc(=O)n(C)c21] | 25 mg, 32% | Compound A35 | ¹H NMR (400 MHz, CD₃OD) δ 7.98 (d, J = 6.0 Hz, 1H), 7.05 (d, J = 9.5 Hz, 1H), 6.05- 5.08 (m, 2H), 4.37 (d, J = 7.2 Hz, 2H), 4.23 (s, 1H), 4.00 (s, 2H), 3.76 (s, 3H), 3.69 (d, J = 6.4 Hz, 1H), 3.65-3.45 (m, 2H), 3.39- 3.32 (m, 0.5H), 2.93-2.72 (m, 2H), 2.30- 2.20 (m, 0.5H), 1.46 (t, J = 6.9 Hz, 6H), 1.36 (d, J = 14.6 Hz, 6H), 1.29 (d, J = 6.1 Hz, 3H), 1.18-0.80 (m, 3H) ppm. MS: M/e 521 (M + 1)⁺ |
| A76 | 2-(6-((2S,5R)-4-(1-(2,2- dimethyl-2,3-dihydro- [1,4]dioxino[2,3-b]pyridin-7- yl)ethyl)-2,5-dimethylpiperazin- 1-yl)-9-ethyl-3-methyl-2-oxo- 3,9-dihydro-2H-purin-8- yl)acetonitrile | 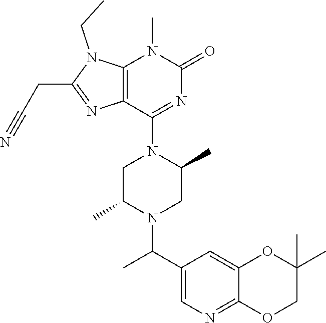 [SMILES: CCn1c(CC#N)nc2c(N3C[C@@H](C)N(C(C)c4cnc5c(c4)OC(C)(C)CO5)C[C@@H]3C)nc(=O)n(C)c21] | 29 mg, 37% | Compound A35 | ¹H NMR (400 MHz, CD₃OD) δ 7.67 (d, J = 4.9 Hz, 1H), 7.32 (s, 1H), 6.56-4.91 (m, 2H), 4.43-4.32 (m, 2H), 4.23 (s, 1H), 4.13 (d, J = 3.3 Hz, 2H), 3.76 (s, 3H), 3.68- 3.46 (m, 2H), 3.36-3.32 (m, 1H), 2.93- 2.60 (m, 2.5H), 2.30-2.20 (m, 0.5H), 1.51- 1.37 (m, 6H), 1.37-1.32 (m, 6H), 1.32- 1.21 (m, 3H), 1.06-0.90 (m, 3H) ppm. MS: M/e 521 (M + 1)⁺ |
| A82 | 2-(6-((2S,5R)-2,5-dimethyl-4-(1- (1-oxo-2-(2,2,2- trifluoroethyl)isoindolin-5- yl)ethyl)piperazin-1-yl)-9-ethyl- 3-methyl-2-oxo-3,9-dihydro-2H- purin-8-yl)acetonitrile | 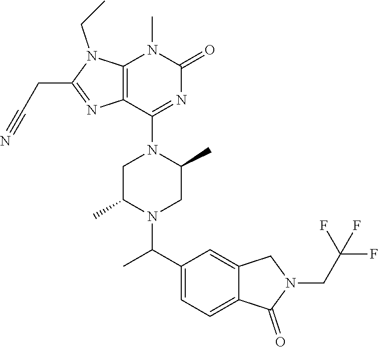 [SMILES: CCn1c(CC#N)nc2c(N3C[C@@H](C)N(C(C)c4ccc5c(c4)CN(CC(F)(F)F)C5=O)C[C@@H]3C)nc(=O)n(C)c21] | 27 mg, 20% | Compound A35 | ¹H NMR (400 MHz, DMSO-d6) δ 7.79 (t, J = 8.0 Hz, 1H), 7.72 (d, J = 9.7 Hz, 1H), 7.62 (d, J = 7.7 Hz, 1H), 6.35-4.88 (m, 2H), 4.68 (d, J = 8.8 Hz, 2H), 4.54-4.39 (m, 4H), 4.32 (s, 2H), 3.83-3.57 (m, 5H), 2.85-2.63 (m, 2H), 2.45-2.11 (m, 1H), 1.43-1.21 (m, 9H), 1.06-0.87 (m, 3H) ppm. MS: M/e 571 (M + 1)⁺. |
| A84 | 2-(6-((2S,5R)-2,5-dimethyl-4-(1- (2,3,3-trimethyl-1-oxoisoindolin- 5-yl)ethyl)piperazin-1-yl)-9- ethyl-3-methyl-2-oxo-3,9- dihydro-2H-purin-8- yl)acetonitrile | 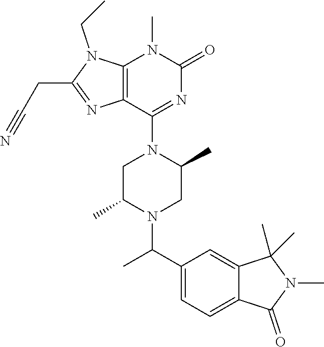 [SMILES: CCn1c(CC#N)nc2c(N3C[C@@H](C)N(C(C)c4ccc5c(c4)C(C)(C)N(C)C5=O)C[C@@H]3C)nc(=O)n(C)c21] | 45 mg, 34% | Compound A35 | ¹H NMR (400 MHz, CD₃OD) δ 7.80- 7.61 (m, 2H), 7.60-7.45 (m, 1H), 6.40- 4.45 (m, 2H), 4.43-4.30 (m, 2H), 4.22 (s, 2H), 3.86-3.49 (m, 5H), 3.04 (s, 3H), 2.98-2.05 (m, 3H), 1.62-1.19 (m, 15H), 1.10-0.85 (m, 3H) ppm. MS: M/e 531 (M + 1)⁺. |
| A89 | 2-(6-((2S,5R)-2,5-dimethyl-4-(1- (3-methyl-2,3-dihydro- [1,4]dioxino[2,3-b]pyridin-6- yl)ethyl)piperazin-1-yl)-9-ethyl- 3-methyl-2-oxo-3,9-dihydro-2H- purin-8-yl)acetonitrile | 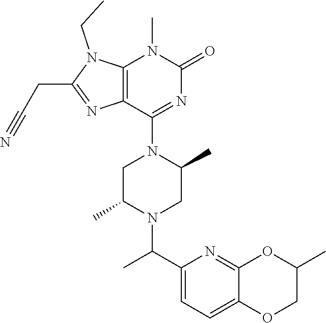 [SMILES: CCn1c(CC#N)nc2c(N3C[C@@H](C)N(C(C)c4ccc5c(n4)OC(C)CO5)C[C@@H]3C)nc(=O)n(C)c21] | 2 mg, 3% | Compound A35 | ¹H NMR (400 MHz, CD₃OD) δ 7.29 (dd, J = 7.7, 3.2 Hz, 1H), 7.11 (t, J = 7.4 Hz, 1H), 5.97 (s, 1H), 5.13 (s, 1H), 4.46 (d, J = 11.5 Hz, 1H), 4.35 (dt, J = 17.0, 7.6 Hz, 3H), 4.21 (s, 1H), 4.09-3.98 (m, 1H), 3.77 (s, 3H), 3.70 (s, 1H), 3.48 (s, 1H), 3.13-2.9 (m, 3H), 2.40-2.00 (m, 1H), 1.46 (t, J = 7.1 Hz, 4H), 1.38-1.29 (m, 8H), 1.14 (s, 1.5H), 1.00 (d, J = 6.0 Hz, 1.5H). ppm. MS: M/e 507 (M + 1)⁺. |
| A90 | 2-(6-((2S,5R)-2,5-dimethyl-4-(1- (2-methylquinolin-7- yl)ethyl)piperazin-1-yl)-9-ethyl- 3-methyl-2-oxo-3,9-dihydro-2H- purin-8-yl)acetonitrile | 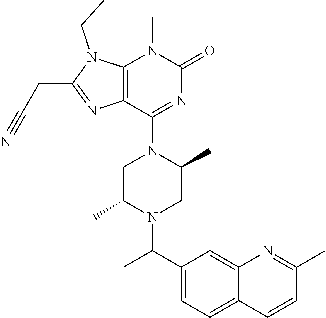 [SMILES: CCn1c(CC#N)nc2c(N3C[C@@H](C)N(C(C)c4ccc5ccc(C)nc5c4)C[C@@H]3C)nc(=O)n(C)c21] | 54 mg | Compound A35 | ¹H NMR (400 MHz, DMSO-d₆) δ 8.27- 8.17 (m, 1H), 7.92 (d, J = 8.5 Hz, 1H), 7.86 (d, J = 9.4 Hz, 1H), 7.69 (d, J = 8.4 Hz, 1H), 7.40 (dd, J = 8.4, 4.4 Hz, 1H), 6.23-4.89 (m, 2H), 4.37 (d, J = 6.9 Hz, 2H), 4.22 (s, 1H), 3.84 (s, 1H), 3.76 (s, 3H), 3.74-3.39 (m, 2H), 3.24 (s, 1H), 2.93 (s, 1H), 2.72 (s, 3H), 2.21 (d, J = 12.0 Hz, 1H), 1.53-1.37 (m, 6H), 1.29 (s, 3H), 1.01 (dd, J = 64.1, 6.2 Hz, 3H) ppm. MS: M/e 499 (M + 1)⁺. |
| A92 | 2-(6-((2S,5R)-2,5-dimethyl-4-(1- (2-methyl-1-oxo-1,2- dihydroisoquinolin-7- yl)ethyl)piperazin-1-yl)-9-ethyl- 3-methyl-2-oxo-3,9-dihydro-2H- purin-8-yl)acetonitrile | 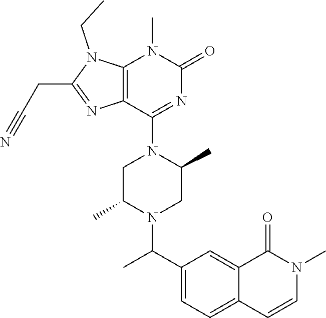 [SMILES: CCn1c(CC#N)nc2c(N3C[C@@H](C)N(C(C)c4ccc5ccn(C)c(=O)c5c4)C[C@@H]3C)nc(=O)n(C)c21] | 103 mg | Compound A35 | ¹H NMR (400 MHz, DMSO-d₆) δ 8.19 (d, J = 14.6 Hz, 1H), 7.74 (dd, J = 14.8, 8.6 Hz, 1H), 7.67-7.59 (m, 1H), 7.44 (t, J = 6.7 Hz, 1H), 6.66-6.51 (m, 1H), 5.75 (s, 1H), 4.86 (s, 1H), 4.41 (s, 2H), 4.25 (s, 2H), 3.74 (q, J = 6.4 Hz, 0.5H), 3.61 (s, 3H), 3.56 (s, 0.5H), 3.50 (s, 3H), 3.32 (s, 2H), 3.31-3.27 (m, 0.5H), 2.76 (s, 1H), 2.09 (d, J = 11.7 Hz, 0.5H), 1.31 (dd, J = 14.5, 8.0 Hz, 7.5H), 1.16 (s, 1.5H), 0.98 (s, 1.5H), 0.83 (s, 1.5H) ppm. MS: M/e 515 (M + 1)⁺. |
| A93 | 2-(6-((2S,5R)-2,5-dimethyl-4-(1- (2-methyl-1-oxo-1,2,3,4- tetrahydro isoquinolin-7- yl)ethyl)piperazin-1-yl)-9-ethyl- 3-methyl-2-oxo-3,9-dihydro-2H- purin-8-yl)acetonitrile | 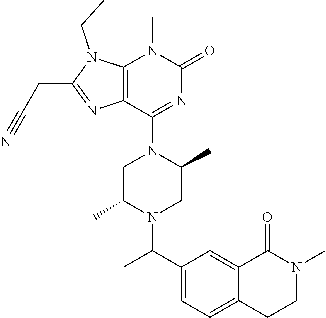 [SMILES: CCn1c(CC#N)nc2c(N3C[C@@H](C)N(C(C)c4ccc5c(c4)C(=O)N(C)CC5)C[C@@H]3C)nc(=O)n(C)c21] | 102 mg | Compound A35 | ¹H NMR (400 MHz, CD₃OD) δ 7.99 (d, J = 4.0 Hz, 1H), 7.53 (d, J = 7.7 Hz, 1H), 7.31- 7.22 (m, 1H), 6.30-5.57 (m, 1H), 5.50- 4.95 (m, 1H), 4.37 (q, J = 7.2 Hz, 2H), 4.23 (s, 2H), 3.77 (s, 0.5H), 3.76 (s, 3H), 3.63 (t, J = 6.2 Hz, 3H), 3.30-3.28 (m, 1.5H), 3.14 (s, 3H), 3.02 (q, J = 8.3 Hz, 2H), 2.95 (t, J = 8.3 Hz, 1H), 2.71 (d, J = 7.6 Hz, 0.5H), 2.25 (d, J = 12.3 Hz, 0.5H), 1.46 (t, J = 6.7 Hz, 4.5H), 1.37 (q, J = 2.7 Hz, 3H), 1.26 (d, J = 5.4 Hz, 1.5H), 1.08 (d, J = 6.2 Hz, 1.5H), 0.94 (d, J = 6.3 Hz, 1.5H) ppm. MS: M/e 517 (M + 1)⁺. |
| A100 | 2-(6-((2S,5R)-2,5-dimethyl-4-(1- (2-methyl-3-oxoisoindolin-5- yl)ethyl)piperazin-1-yl)-9-ethyl- 3-methyl-2-oxo-3,9-dihydro-2H- purin-8-yl)acetonitrile | 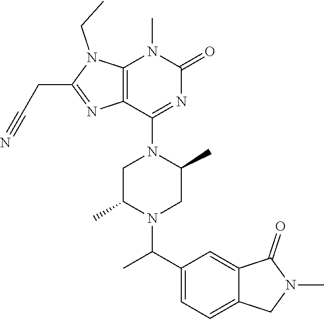 [SMILES: CCn1c(CC#N)nc2c(N3C[C@@H](C)N(C(C)c4ccc5c(c4)C(=O)N(C)C5)C[C@@H]3C)nc(=O)n(C)c21] | 66 mg | Compound A35 | ¹H NMR (400 MHz, DMSO-d₆) δ 7.69- 7.50 (m, 3H), 6.20-5.30 (m, 1H), 5.20-4.55 (m, 1H), 4.43 (s, 4H), 4.25 (s, 2H), 3.70 (q, J = 6.0 Hz, 0.5H), 3.60 (s, 3H), 3.58-3.50 (m, 1H), 3.33 (s, 2H), 3.07 (s, 3H), 2.78 (s, 1H), 2.07 (d, J = 11.1 Hz, 0.5H), 1.29 (dt, J = 18.5, 12.6 Hz, 7.5H), 1.14 (s, 1.5H), 0.97 (s, 1.5H), 0.82 (d, J = 5.9 Hz, 1.5H) ppm. MS: M/e 503 (M + 1)⁺. |
| A101 | 2-(6-((2S,5R)-2,5-dimethyl-4-(1- (2-methyl-1-oxoisoindolin-5- yl)ethyl) piperazin-1-yl)-9-ethyl- 3-methyl-2-oxo-3,9-dihydro-2H- purin-8-yl)acetonitrile | 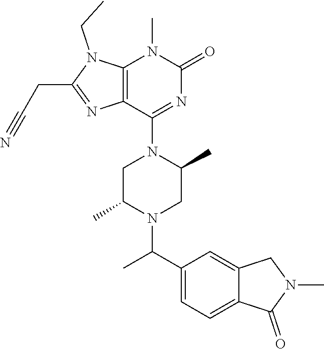 [SMILES: CCn1c(CC#N)nc2c(N3C[C@@H](C)N(C(C)c4ccc5c(c4)CN(C)C5=O)C[C@@H]3C)nc(=O)n(C)c21] | 70 mg | Compound A35 | ¹H NMR (400 MHz, DMSO-d₆) δ 7.61 (dd, J = 18.8, 10.3 Hz, 2H), 7.50 (t, J = 6.6 Hz, 1H), 6.10-5.40 (m, 1H), 5.20-4.55 (m, 1H), 4.53-4.33 (m, 4.5H), 4.25 (s, 2H), 3.69 (q, J = 6.6 Hz, 1H), 3.61 (s, 3H), 3.56 (q, J = 6.3 Hz, 1H), 3.33 (s, 1H), 3.06 (s, 3H), 2.78 (s, 1H), 2.08 (d, J = 12.2 Hz, 0.5H), 1.40-1.24 (m, 7.5H), 1.17 (s, 1.5H), 0.97 (s, 1.5H), 0.83 (d, J = 5.5 Hz, 1.5H) ppm. MS: M/e 503 (M + 1)⁺. |
| A104 | 2-(6-((2S,5R)-2,5-dimethyl-4-(1- (2-(trifluoromethyl)thiazol-4- yl)ethyl)piperazin-1-yl)-9-ethyl- 3-methyl-2-oxo-3,9-dihydro-2H- purin-8-yl)acetonitrile | 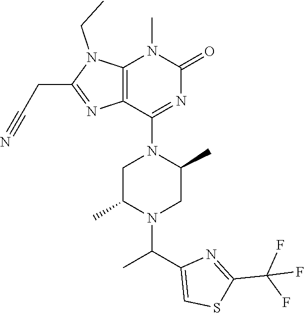 [SMILES: CCn1c(CC#N)nc2c(N3C[C@@H](C)N(C(C)c4csc(C(F)(F)F)n4)C[C@@H]3C)nc(=O)n(C)c21] | 44 mg | Compound A35 | ¹H NMR (400 MHz, CD₃OD) δ 7.86 (s, 1H), 6.15-4.93 (m, 2H), 4.37 (q, J = 7.2 Hz, 2H), 4.24 (s, 2H), 3.77 (s, 3H), 3.74- 3.37 (m, 2H), 3.25-2.75 (m, 2H), 2.73- 2.10 (m, 1H), 1.50-1.43 (m, 5H), 1.41- 1.25 (m, 4H), 1.22-0.93 (m, 3H) ppm. MS: M/e 509(M + 1)⁺. |
| A105 | 2-(6-((2S,5R)-2,5-dimethyl-4-(1- (2-(trifluoromethyl)thiazol-5- yl)ethyl)piperazin-1-yl)-9-ethyl- 3-methyl-2-oxo-3,9-dihydro-2H- purin-8-yl)acetonitrile | 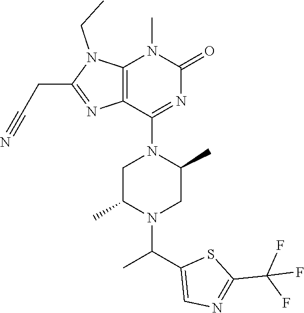 [SMILES: CCn1c(CC#N)nc2c(N3C[C@@H](C)N(C(C)c4cnc(C(F)(F)F)s4)C[C@@H]3C)nc(=O)n(C)c21] | 30 mg | Compound A35 | ¹H NMR (400 MHz, CD₃OD) δ 7.86 (s, 1H), 6.38-4.96 (m, 2H), 4.42-4.33 (m, 2H), 4.29-4.04 (m, 2H), 3.77 (s, 3H), 3.67-3.43 (m, 1H), 3.31-3.29 (m, 1H), 3.19-2.96 (m, 1H), 2.83-2.38 (m, 2H), 1.53-1.21 (m, 9H), 1.07 (d, J = 6.4 Hz, 3H) ppm. MS: M/e 509 (M + 1)⁺. |
| A106 | 2-(6-((2S,5R)-2,5-dimethyl-4-(1- (1-methyl-6-oxo-1,6- dihydropyridin-3- yl)ethyl)piperazin-1-yl)-9-ethyl- 3-methyl-2-oxo-3,9-dihydro-2H- purin-8-yl)acetonitrile | 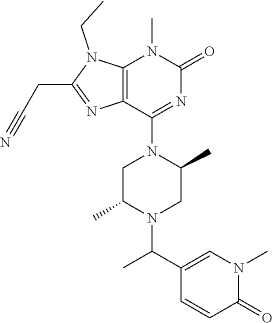 [SMILES: CCn1c(CC#N)nc2c(N3C[C@@H](C)N(C(C)c4ccc(=O)n(C)c4)C[C@@H]3C)nc(=O)n(C)c21] | 60 mg, 44% | Compound A35 | ¹H NMR (400 MHz, CD₃OD) δ 7.70 (t, J = 8.0 Hz, 1H), 7.67-7.57 (m, 1H), 6.59 (t, J = 10.0 Hz, 1H), 6.25-5.62 (m, 1H), 5.31- 4.95 (m, 1H), 4.44-4.30 (m, 2H), 4.28- 4.18 (m, 2H), 3.76 (s, 3H), 3.71-3.53 (m, 4.5H), 3.49-3.41 (m, 1H), 3.14-2.94 (m, 1H), 2.89-2.70 (m, 1H), 2.43-2.36 (m, 0.5H), 1.51-1.23 (m, 9H), 1.14-0.95 (m, 3H) ppm. MS: M/e 465 (M + 1)⁺. |
| A107 | 2-(6-((2S,5R)-2,5-dimethyl-4-(1- (1-methyl-2-oxo-1,2- dihydropyridin-4- yl)ethyl)piperazin-1-yl)-9-ethyl- 3-methyl-2-oxo-3,9-dihydro-2H- purin-8-yl)acetonitrile | 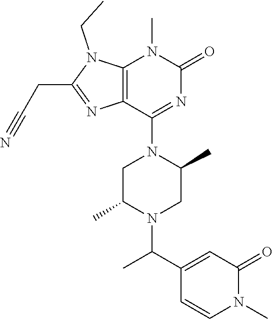 [SMILES: CCn1c(CC#N)nc2c(N3C[C@@H](C)N(C(C)c4ccn(C)c(=O)c4)C[C@@H]3C)nc(=O)n(C)c21] | 40 mg | Compound A35 | ¹H NMR (400 MHz, CD₃OD) δ 7.62 (t, J = 6.8 Hz, 1H), 6.59-6.51 (m, 2H), 6.26- 4.99 (m, 2H), 4.45-4.31 (m, 2H), 4.24 (s, 2H), 3.76 (s, 3H), 3.58-3.34 (m, 2.5H), 3.01-2.70 (m, 2H), 2.66 (s, 3H), 2.25 (d, J = 12.4 Hz, 0.5H), 1.53-1.24 (m, 9H), 0.98 (dd, J = 36.4, 6.4 Hz, 3H) ppm. MS: M/e 465 (M + 1)⁺. |
| A109 | 2-(6-((2S,5R)-4-(1-(3- (difluoromethyl)phenyl)ethyl)- 2,5-dimethylpiperazin-1-yl)-9- ethyl-3-methyl-2-oxo-3,9- dihydro-2H-purin-8- yl)acetonitrile | 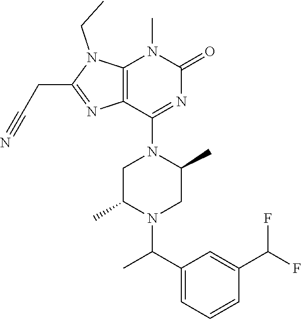 [SMILES: CCn1c(CC#N)nc2c(N3C[C@@H](C)N(C(C)c4cccc(C(F)F)c4)C[C@@H]3C)nc(=O)n(C)c21] | 10 mg | Compound A35 | ¹H NMR (400 MHz, CD₃OD) δ 7.64- 7.37 (m, 4H), 6.75 (td, J = 56.3, 10.3 Hz, 1H), 6.12-5.01 (m, 2H), 4.43-4.31 (m, 2H), 4.22 (s, 2H), 3.76 (s, 3H), 3.74-3.45 (m, 2H), 3.24 (s, 1H), 3.01-2.78 (m, 1H), 2.68-2.03 (m, 1H), 1.56-1.19 (m, 9H), 1.12-0.85 (m, 3H) ppm. MS: M/e 484 (M + 1)⁺. |
| A111 | 2-(9-ethyl-6-((2S,5R)-4-(1-(3-(2- hydroxypropan-2- yl)phenyl)ethyl)-2,5- dimethylpiperazin-1-yl)-3- methyl-2-oxo-3,9-dihydro-2H- purin-8-yl)acetonitrile | 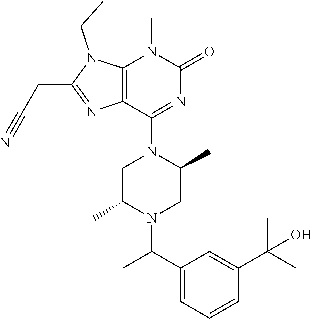 [SMILES: CCn1c(CC#N)nc2c(N3C[C@@H](C)N(C(C)c4cccc(C(C)(C)O)c4)C[C@@H]3C)nc(=O)n(C)c21] | 70 mg, 47% | Compound A35 | ¹H NMR (400 MHz, CD₃OD) δ 7.60 (d, J = 9.2 Hz, 1H), 7.50-7.24 (m, 3H), 6.20- 5.70 (m, 1H), 5.25-4.95 (m, 1H), 4.37 (q, J = 7.2 Hz 2H), 4.28-4.17 (m, 1.5H), 4.00-3.90 (m, 0.5H), 3.82-3.71 (m, 4H), 3.60-3.41 (m, 0.5H), 3.35-3.30 (m, 1H), 3.21-3.00 (m, 1.5H), 2.92-2.80 (m, 0.5H), 2.65 (brs, 1H), 2.44-2.34 (m, 0.5H), 1.60-1.40 (m, 13.5H), 1.35-0.99 (m, 4.5H) ppm. MS: M/e 492 (M + 1)⁺. |
| A114 | 2-(9-ethyl-6-((2S,5R)-4-(1-(2- fluoro-5- (trifluoromethyl)phenyl)ethyl)- 2,5-dimethylpiperazin-1-yl)-3- methyl-2-oxo-3,9-dihydro-2H- purin-8-yl)acetonitrile | 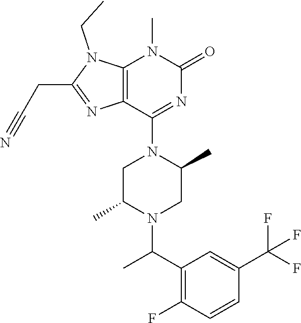 [SMILES: CCn1c(CC#N)nc2c(N3C[C@@H](C)N(C(C)c4cc(C(F)(F)F)ccc4F)C[C@@H]3C)nc(=O)n(C)c21] | 27 mg | Compound A35 | ¹H NMR (400 MHz, CD₃OD) δ 8.09- 7.88 (m, 1H), 7.63-7.55 (m, 1H), 7.33- 7.19 (m, 1H), 6.40-4.89 (m, 2H), 4.37 (q, J = 7.1 Hz, 2H), 4.22 (s, 1H), 4.17-3.82 (m, 1H), 3.76 (s, 3H), 3.68-3.39 (m, 1H), 3.31-3.14 (m, 1H), 3.10-2.74 (m, 2H), 2.72-2.03 (m, 1H), 1.49-1.41 (m, 4H), 1.40-1.25 (m, 5H), 1.09-0.90 (m, 3H) ppm. MS: M/e 520(M + 1)⁺. |
| A116 | 2-(6-((2S,5R)-2,5-dimethyl-4-(1- (4-methyl-2- (trifluoromethyl)phenyl)ethyl)pi- perazin-1-yl)-9-ethyl-3-methyl- 2-oxo-3,9-dihydro-2H-pyrin-8- yl)acetonitrile | 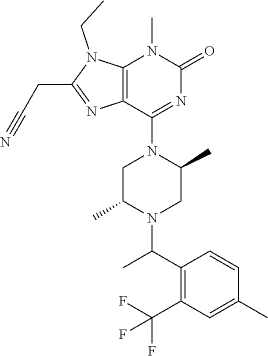 [SMILES: CCn1c(CC#N)nc2c(N3C[C@@H](C)N(C(C)c4ccc(C)cc4C(F)(F)F)C[C@@H]3C)nc(=O)n(C)c21] | A116 (37 mg) A116a (20 mg) A116b (16 mg) | Compound 35 | Compound A116: ¹H NMR (400 MHz, CD₃OD) δ 7.99-7.76 (m, 1H), 7.51-7.28 (m, 2H), 6.26-4.53 (m, 2H), 4.37 (dd, J = 14.7, 7.2 Hz, 2H), 4.22 (d, J = 23.3 Hz, 1H), 4.12-3.79 (m, 1H), 3.76 (s, 3H), 3.69- 3.41 (m, 1H), 3.30-3.07 (m, 1H), 3.02- 2.58 (m, 2H), 2.39 (s, 3H), 2.07-1.52 (m, 1H), 1.51-1.40 (m, 4H), 1.35-1.16 (m, 5H), 1.11-0.80 (m, 3H) ppm. MS: M/e 516 (M + 1)⁺. Compound A116a (the earlier peak): ¹H NMR (400 MHz, CD₃OD) δ 7.88 (d, J = 7.8 Hz, 1H), 7.43 (s, 2H), 5.92 (d, J = 13.2 Hz, 1H), 4.97 (d, J = 51.1 Hz, 1H), 4.83 (s, 1H), 4.37 (d, J = 6.9 Hz, 2H), 4.26-3.78 (m, 2H), 3.76 (s, 3H), 3.71-3.37 (m, 2H), 2.81- 2.58 (m, 1H), 2.39 (s, 3H), 2.02 (d, J = 11.9 Hz, 1H), 1.46 (t, J = 6.8 Hz, 3H), 1.38- 1.14 (m, 6H), 1.04 (s, 3H) ppm. MS: M/e 516 (M + 1)⁺. Compound A116b (the later peak): ¹H NMR (400 MHz, CD₃OD) δ 7.93 (d, J = |
| | | | | | 8.1 Hz, 1H), 7.46 (s, 2H), 6.30-4.58 (m, |
| | | | | | 2H), 4.47-4.31 (m, 2H), 4.28-3.81 (m, |
| | | | | | 2H), 3.76 (s, 3H), 3.54 (d, J = 13.7 Hz, |
| | | | | | 1H), 3.27-3.06 (m, 1H), 3.01-2.71 (m, |
| | | | | | 3H), 2.40 (s, 3H), 1.59-1.39 (m, 6H), |
| | | | | | 1.24 (s, 3H), 0.88 (s, 3H) ppm. MS: M/e |
| | | | | | 516 (M + 1)⁺. |
| A118 | 2-(6-((2S,5R)-2,5-dimethyl-4-(1- (6-(trifluoromethyl)pyridin-2- yl)ethyl)piperazin-1-yl)-9-ethyl- 3-methyl-2-oxo-3,9-dihydro-2H- purin-8-yl)acetonitrile | 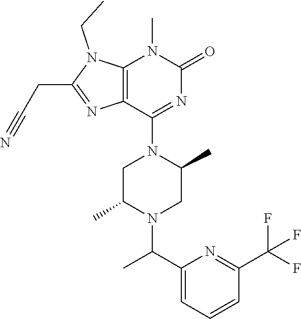 [SMILES: CCn1c(CC#N)nc2c(N3C[C@@H](C)N(C(C)c4cccc(C(F)(F)F)n4)C[C@@H]3C)nc(=O)n(C)c21] | A118a (38 mg) A118b (63 mg) | Compound A35 | Compound A118a (the earlier peak): ¹H NMR (400 MHz, DMSO-d₆) δ 8.10 (t, J = 7.7 Hz, 1H), 7.87 (d, J = 8.0 Hz, 1H), 7.78 (d, J = 7.6 Hz, 1H), 5.73 (s, 1H), 4.89 (s, 1H), 4.40 (s, 2H), 4.26 (q, J = 7.0 Hz, 2H), 3.67 (q, J = 6.6 Hz, 2H), 3.61 (s, 3H), 3.51 (s, 1H), 2.67 (s, 1H), 2.04 (d, J = 11.8 Hz, 1H), 1.33 (dd, J = 18.1, 6.8 Hz, 6H), 1.19 (s, 3H), 0.96 (d, J = 5.3 Hz, 3H) ppm. MS: M/e 503 (M + 1)⁺. Compound A118b (the later peak): ¹H NMR (400 MHz, DMSO-d₆) δ 8.10 (t, J = 7.7 Hz, 1H), 7.88 (d, J = 8.0 Hz, 1H), 7.78 (d, J = 7.6 Hz, 1H), 6.23 - 4.52 (m, 2H), 4.39 (s, 2H), 4.26 (q, J = 6.6 Hz, 2H), 3.87 (q, J = 6.6 Hz, 1H), 3.61 (s, 3H), 3.32 (s, 1H), 2.83 (d, J = 20.6 Hz, 2H), 2.72 (s, 1H), 1.33 (dd, J = 16.7, 6.9 Hz, 9H), 0.89 (d, J = 6.2 Hz, 3H) ppm. MS: M/e 503 (M + 1)⁺. |
| A119 | 2-(6-((2S,5R)-2,5-dimethyl-4-(1- (2-(trifluoromethyl)pyridin-4- yl)ethyl)piperazin-1-yl)-9-ethyl- 3-methyl-2-oxo-3,9-dihydro-2H- purin-8-yl)acetonitrile | 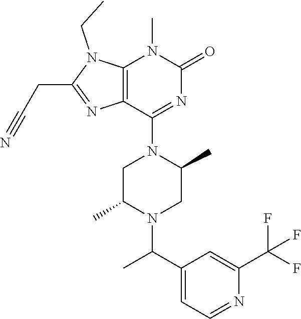 [SMILES: CCn1c(CC#N)nc2c(N3C[C@@H](C)N(C(C)c4ccnc(C(F)(F)F)c4)C[C@@H]3C)nc(=O)n(C)c21] | 126 mg | Compound A35 | ¹H NMR (400 MHz, DMSO-d₆) δ 8.72 (t, J = 5.8 Hz, 1H), 7.92 (s, 1H), 7.75 (dd, J = 22.0, 4.9 Hz, 1H), 6.10-4.60 (m, 2H), 4.40 (s, 2H), 4.26 (q, J = 6.7 Hz, 2H), 3.86 (q, J = 6.6 Hz, 0.5H), 3.66 (q, J = 6.7 Hz, 1H), 3.61 (s, 3H), 3.52 (s, 0.5H), 3.33 (s, 0.5H), 2.79 (s, 1H), 2.67 (s, 1H), 2.03 (s, 0.5H), 1.34 (t, J = 7.0 Hz, 4.5H), 1.28 (t, J = 6.4 Hz, 3H), 1.19 (s, 1.5H), 0.97 (d, J = 5.5 Hz, 1.5H), 0.89 (d, J = 5.8 Hz, 1.5H) ppm. MS: M/e 503 (M + 1)⁺. |
| A120 | 2-(6-((2S,5R)-2,5-dimethyl-4-(1- (4-(trifluoromethyl)pyridin-2- yl)ethyl)piperazin-1-yl)-9-ethyl- 3-methyl-2-oxo-3,9-dihydro-2H- purin-8-yl)acetonitrile | 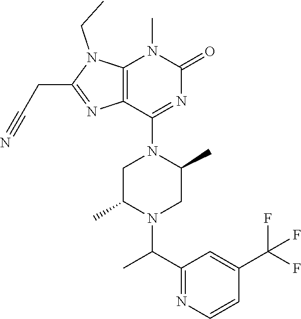 [SMILES: CCn1c(CC#N)nc2c(N3C[C@@H](C)N(C(C)c4cc(C(F)(F)F)ccn4)C[C@@H]3C)nc(=O)n(C)c21] | 108 mg | Compound A35 | ¹H NMR (400 MHz, DMSO-d₆) δ 8.78 (dd, J = 15.1, 5.0 Hz, 1H), 7.88 (d, J = 5.8 Hz, 1H), 7.66 (d, J = 4.7 Hz, 1H), 6.10-5.30 (m, 1H), 5.20-4.50 (m, 1H), 4.40 (s, 2H), 4.26 (q, J = 7.1 Hz, 2H), 3.96 (q, J = 6.3 Hz, 0.5H), 3.68 (q, J = 6.8 Hz, 0.5H), 3.61 (s, 3H), 3.53 (s, 0.5H), 3.33-3.27 (m, 1H), 2.88 (s, 1H), 2.67 (s, 1H), 2.02 (d, J = 12.1 Hz, 0.5H), 1.38-1.27 (m, 7.5H), 1.19 (s, 1.5H), 1.01-0.89 (m, 3H) ppm. MS: M/e 503 (M + 1)⁺. |
| A121 | 2-(6-((2S,5R)-4-(1-(2-(azetidin- 1-yl)pyridin-4-yl)ethyl)-2,5- dimethyl piperazin-1-yl)-9-ethyl- 3-methyl-2-oxo-3,9-dihydro-2H- purin-8-yl)acetonitrile | 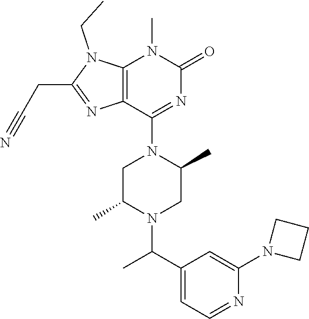 [SMILES: CCn1c(CC#N)nc2c(N3C[C@@H](C)N(C(C)c4ccnc(N5CCC5)c4)C[C@@H]3C)nc(=O)n(C)c21] | 16 mg, 25% | Compound A35 | ¹H NMR (400 MHz, CD₃OD) δ 7.92- 7.84 (m, 1H), 6.81 (s, 1H), 6.57 (d, J = 16.2 Hz, 1H), 6.02 (m, 1H), 5.32-4.99 (m, 1H), 4.37 (d, J = 7.6 Hz, 2H), 4.23 (s, 2H), 4.12 (s, 4H), 3.76 (s, 3H), 3.60 (m, 1H), 3.47 (s, 1H), 2.87 (m, 2H), 2.75- 2.65 (m, 0.5H), 2.46 (s, 2H), 2.22 (d, J = 11.4 Hz, 0.5H), 1.46 (t, J = 6.8 Hz, 4H), 1.31 (dd, J = 12.5, 6.5 Hz, 5H), 1.04 (d, J = 6.6 Hz, 1.5H), 0.94 (d, J = 6.4 Hz, 1.5H) ppm. MS: M/e 490 (M + 1)⁺. |
| A122 | 2-(9-ethyl-6-((2S,5R)-4-(1-(2- (isopropylamino)pyridin-4- yl)ethyl)-2,5-dimethylpiperazin- 1-yl)-3-methyl-2-oxo-3,9- dihydro-2H-purin-8- yl)acetonitrile | 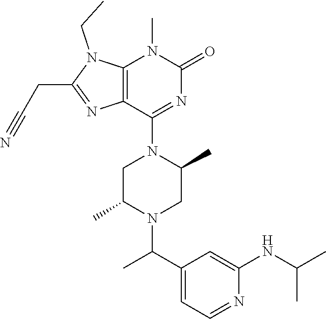 | 19 mg, 30% | Compound A35 | ¹H NMR (400 MHz, CD₃OD) δ 7.86-7.79 (m, 1H), 6.73 (s, 1H), 6.70 (s, 1H), 5.90 (m, 1H), 5.37-4.98 (m, 1H), 4.37 (d, J = 7.0 Hz, 2H), 4.23 (s, 1H), 3.92 (dd, J = 12.9, 6.4 Hz, 1H), 3.76 (s, 3H), 3.55 (d, J = 6.1 Hz, 1H), 3.41 (d, J = 6.6 Hz, 1H), 3.32 (s, 1H), 2.88 (d, J = 47.2 Hz, 2H), 2.68 (s, 0.5H), 2.24 (d, J = 12.5 Hz, 0.5H), 1.46 (t, J = 7.0 Hz, 4.5H), 1.35-1.23 (m, 10.5H), 1.03 (d, J = 6.2 Hz, 1.5H), 0.93 (d, J = 6.2 Hz, 1.5H) ppm. MS: M/e 507 (M + 1)⁺. |
| A127 | 2-(6-((2S,5R)-2,5-dimethyl-4-(1- (3-(oxetan-3- yl)phenyl)ethyl)piperazin-1-yl)- 9-ethyl-3-methyl-2-oxo-3,9- dihydro-2H-purin-8- yl)acetonitrile | 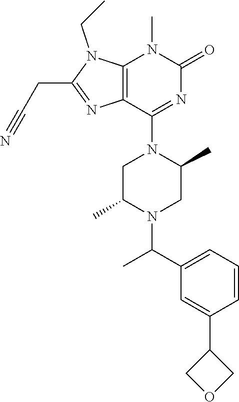 [SMILES: CCn1c(CC#N)nc2c(N3C[C@@H](C)N(C(C)c4cccc(C5COC5)c4)C[C@@H]3C)nc(=O)n(C)c21] | 73 mg, 50% | Compound A35 | ¹H NMR (400 MHz, CD₃OD) δ 7.56-7.25 (m, 4H), 6.39-5.45 (m, 1H), 5.26-4.95 (m, 2H), 4.79-4.72 (m, 2H), 4.45-4.17(m, 4H), 3.95-3.65 (m, 5H), 3.57-3.42 (m, 0.5H), 3.35-3.31 (m, 1H), 3.16-2.90 (m, 1.5H), 2.88-2.76 (m, 0.5H), 2.68-2.63 (m, 1H), 2.42-2.27 (m, 0.5H), 1.53-1.40 (m, 8H), 1.35-0.95 (m, 4H) ppm. MS: M/e 490 (M + 1)⁺. |
| A128 | 2-(6-((2S,5R)-2,5-dimethyl-4-(1- (6-(trifluoromethoxy)pyridin-2- yl)ethyl)piperazin-1-yl)-9-ethyl- 3-methyl-2-oxo-3,9-dihydro-2H- purin-8-yl)acetonitrile | 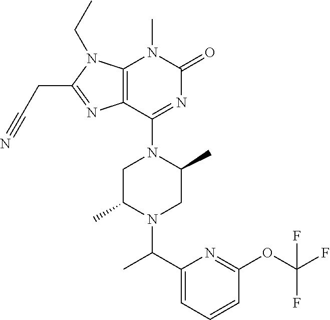 [SMILES: CCn1c(CC#N)nc2c(N3C[C@@H](C)N(C(C)c4cccc(OC(F)(F)F)n4)C[C@@H]3C)nc(=O)n(C)c21] | 40 mg, 51% | Compound A35 | ¹H NMR (400 MHz, CD₃OD) δ 7.92 (s, 1H), 7.55 (dd, J = 20.6, 7.2 Hz, 1H), 7.02 (d, J = 8.2 Hz, 1H), 6.34-4.89 (m, 2H), 4.37 (d, J = 7.0 Hz, 2H), 4.23 (s, 1H), 3.84 (d, J = 7.2 Hz, 1H), 3.76 (s, 3H), 3.71- 3.32 (m, 3H), 3.03-2.74 (m, 2H), 1.46 (t, J = 6.8 Hz, 3H), 1.40-1.28 (m, 6H), 1.08- 0.95 (m, 3H) ppm. MS: M/e 519 (M + 1)⁺. |
| A130 | 4-(1-((2R,5S)-4-(8- (cyanomethyl)-9-ethyl-3-methyl- 2-oxo-3,9-dihydro-2H-purin-6- yl)-2,5-dimethylpiperazin-1- yl)ethyl)-N-methylpicolinamide | 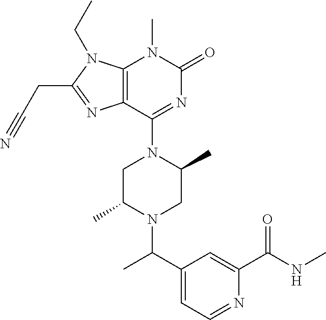 [SMILES: CCn1c(CC#N)nc2c(N3C[C@@H](C)N(C(C)c4ccnc(C(=O)NC)c4)C[C@@H]3C)nc(=O)n(C)c21] | 55 mg, 37% | Compound A35 | ¹H NMR (400 MHz, CD₃OD) δ 8.55 (d, J = 6.4 Hz, 1H), 8.16 (s, 1H), 7.59 (s, 1H), 6.40-5.50 (m, 1H), 5.40-4.50 (m, 1H), 4.45-4.30 (m, 2H), 4.28-3.44 (m, 7H), 2.99-2.06 (m, 6H), 1.49-1.27 (m, 9H), 1.12-0.88 (m, 3H) ppm. MS: M/e 492 (M + 1)⁺ |
| A131 | 4-(1-((2R,5S)-4-(8- (cyanomethyl)-9-ethyl-3-methyl- 2-oxo-3,9-dihydro-2H-purin-6- yl)-2,5-dimethylpiperazin-1- yl)ethyl)-N,N- dimethylpicolinamide | 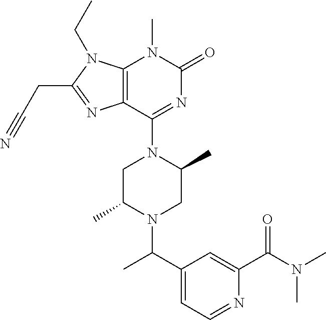 [SMILES: CCn1c(CC#N)nc2c(N3C[C@@H](C)N(C(C)c4ccnc(C(=O)N(C)C)c4)C[C@@H]3C)nc(=O)n(C)c21] | 52 mg, 34% | Compound A35 | ¹H NMR (400 MHz, CD₃OD) δ 8.52 (s, 1H), 7.64 (s, 1H), 7.58 (s, 1H), 6.45-5.45 (m, 1H), 5.40-4.45 (m, 1H), 4.45-4.30 (m, 2H), 4.23 (s, 2H), 3.82-3.54 (m, 5H), 3.14 (s, 3H), 3.04-2.10 (m, 6H), 1.50- 1.27 (m, 9H), 1.10-0.90 (m, 3H) ppm. MS: M/e 506 (M + 1)⁺. |
| A132 | 5-(1-((2R,5S)-4-(8- (cyanomethyl)-9-ethyl-3-methyl- 2-oxo-3,9-dihydro-2H-purin-6- yl)-2,5-dimethylpiperazin-1- yl)ethyl)-2-fluoro-N- methylbenzamide | 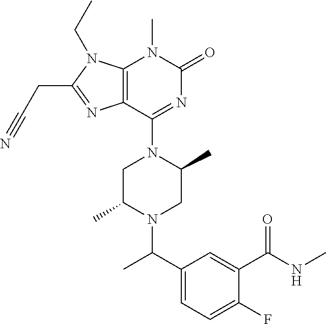 [SMILES: CCn1c(CC#N)nc2c(N3C[C@@H](C)N(C(C)c4ccc(F)c(C(=O)NC)c4)C[C@@H]3C)nc(=O)n(C)c21] | 42 mg | Compound A35 | ¹H NMR (400 MHz, CD₃OD) δ 7.79 (s, 1H), 7.58 (s, 1H), 7.20 (q, J = 10.0 Hz, 1H), 6.10-5.00 (m, 2H), 4.45-4.30 (m, 2H), 4.23 (s, 2H), 3.92-3.14 (m, 6H), 3.05-2.20 (m, 6H), 1.49-1.24 (m, 9H), 1.13-0.90 (m, 3H) ppm. MS: M/e 509 (M + 1)⁺ |
| A133 | 2-(9-ethyl-6-((2S,5R)-4-(1-(3-(1- hydroxy-2-methylpropan-2- yl)phenyl)ethyl)-2,5- dimethylpiperazin-1-yl)-3- methyl-2-oxo-3,9-dihydro-2H- purin-8-yl)acetonitrile | 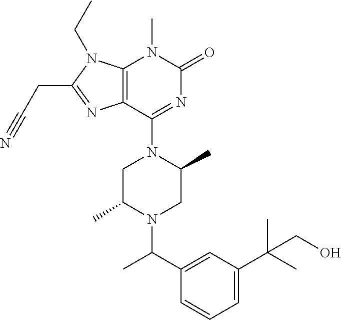 [SMILES: CCn1c(CC#N)nc2c(N3C[C@@H](C)N(C(C)c4cccc(C(C)(C)CO)c4)C[C@@H]3C)nc(=O)n(C)c21] | 38 mg, 25% | Compound A35 | ¹H NMR (400 MHz, CD₃OD) δ 7.49 (s, 1H), 7.41-7.15 (m, 3H), 6.41-5.45 (m, 1H), 5.41-4.92 (m, 1H), 4.43-4.31 (m, 2H), 4.27-4.18 (m, 1H), 3.91-3.80 (m, 1H), 3.76 (s, 3H), 3.74-3.61 (m, 1.5H), 3.67-3.55 (m, 2H), 3.35-3.31 (m, 1H), 3.15-2.90 (m, 1.5H), 2.82-2.71 (m, 0.5H), 2.41-2.30 (m, 0.5H), 1.54-1.37 (m, 7.5H), 1.32 (s, 6H), 1.31-1.24 (m, 1.5H), 1.11 (d, J = 5.6 Hz, 1.5H), 0.99 (d, J = 5.6 Hz, 1.5H) ppm. MS: M/e 506 (M + 1)⁺. |
| A138 | 2-(6-((2S,5R)-4-(1-(5,6- dimethylpyridin-2-yl)ethyl)-2,5- dimethylpiperazin-1-yl)-9-ethyl- 3-methyl-2-oxo-3,9-dihydro-2H- purin-8-yl)acetonitrile | 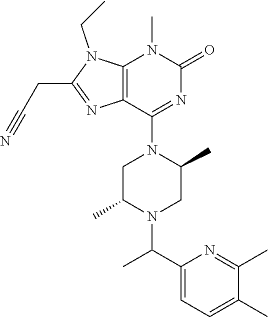 [SMILES: CCn1c(CC#N)nc2c(N3C[C@@H](C)N(C(C)c4ccc(C)c(C)n4)C[C@@H]3C)nc(=O)n(C)c21] | 35 mg | Compound A35 | ¹H NMR (400 MHz, CD₃OD) δ 7.56 (s, 1H), 7.39 (s, 1H), 6.12-4.98 (m, 2H), 4.37 (d, J = 6.9 Hz, 2H), 3.76 (s, 3H), 3.74-3.33 (m, 4H), 3.03-2.59 (m, 2H), 2.48 (d, J = 4.4 Hz, 3H), 2.30 (s, 3H), 2.11 (s, 1H), 1.57- 1.19 (m, 9H), 1.11-0.87 (m, 3H) ppm. MS: M/e 463 (M + 1)⁺. |
| A139 | 2-(6-((2S,5R)-2,5-dimethyl-4-(1- (4- (morpholinomethyl)phenyl)ethyl) piperazin-1-yl)-9-ethyl-3- methyl-2-oxo-3,9-dihydro-2H- purin-8-yl)acetonitrile | 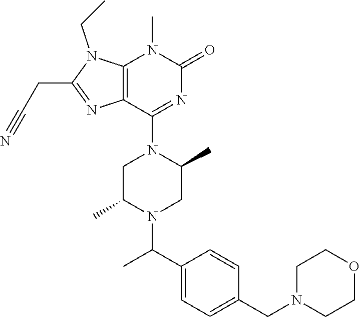 [SMILES: CCn1c(CC#N)nc2c(N3C[C@@H](C)N(C(C)c4ccc(CN5CCOCC5)cc4)C[C@@H]3C)nc(=O)n(C)c21] | 51 mg | Compound A35 | ¹H NMR (400 MHz, DMSO-d₆): δ 7.29 (dt, J = 16.8, 7.9 Hz, 4H), 6.10-5.40 (m, 1H), 5.15-4.55 (m, 1H), 4.40 (s, 2H), 4.26 (d, J = 7.2 Hz, 2H), 3.61 (s, 3H), 3.57 (t, J = 7.2 Hz, 4H), 3.54-3.47 (m, 1H), 3.43 (d, J = 7.2 Hz, 2H), 3.42-3.38 (m, 0.5H), 3.33 (s, 2H), 2.76 (s, 1H), 2.34 (s, 4H), 2.12 (d, J = 12.4 Hz, 0.5H), 1.41-1.12 (m, 9H), 0.94 (s, 1.5H), 0.80 (d, J = 6.2 Hz, 1.5H) ppm. MS: M/e 533 (M + 1)⁺. |
| A140 | 2-(6-((2S,5R)-4-(1-(3-(2-oxa-6- azaspiro[3.3]heptan-6- yl)phenyl)ethyl)-2,5- dimethylpiperazin-1-yl)-9-ethyl- 3-methyl-2-oxo-3,9-dihydro-2H- purin-8-yl)acetonitrile | 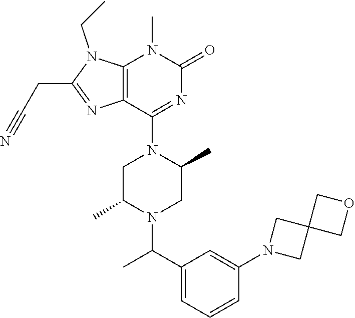 [SMILES: CCn1c(CC#N)nc2c(N3C[C@@H](C)N(C(C)c4cccc(N5CC6(COC6)C5)c4)C[C@@H]3C)nc(=O)n(C)c21] | 8 mg | Compound A35 | ¹H NMR (400 MHz, DMSO-d₆): δ 7.11 (dt, J = 18.4, 7.7 Hz, 1H), 6.69 (dd, J = 12.9, 7.2 Hz, 1H), 6.45 (d, J = 15.3 Hz, 1H), 6.30 (t, J = 8.7 Hz, 1H), 6.05-5.44 (m, 1H), 5.15-4.80 (m, 1H), 4.71 (s, 4H), 4.41 (s, 2H), 4.25 (s, 2H), 4.00-3.90 (m, 4H), 3.68-3.59 (m, 3.5H), 3.53-3.40 (m, 2H), 3.26-3.10 (m, 1H), 2.85-2.72 (m, 1H), 2.19 (d, J = 11.2 Hz, 0.5H), 1.34 (t, J = 7.6 Hz, 4.5H), 1.21 (t, J = 7.9 Hz, 4.5H), 0.94 (s, 1.5H), 0.80 (d, J = 6.6 Hz, 1.5H) ppm. MS: M/e 531 (M + 1)⁺. |
| A141 | 2-(6-((2S,5R)-2,5-dimethyl-4-(1- (3- morpholinophenyl)ethyl)piperazin- 1-yl)-9-ethyl-3-methyl-2-oxo- 3,9-dihydro-2H-purin-8- yl)acetonitrile | 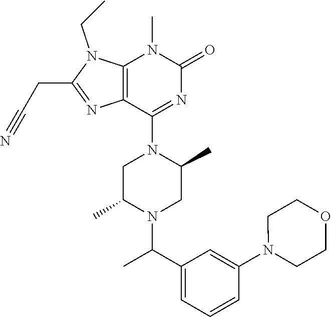 [SMILES: CCn1c(CC#N)nc2c(N3C[C@@H](C)N(C(C)c4cccc(N5CCOCC5)c4)C[C@@H]3C)nc(=O)n(C)c21] | 19 mg | Compound A35 | ¹H NMR (400 MHz, DMSO-d₆): δ 7.23- 7.13 (m, 1H), 6.96 (d, J = 10.1 Hz, 1H), 6.82 (dd, J = 15.5, 7.6 Hz, 2H), 6.10-5.40 (m, 1H), 5.20-4.60 (m, 1H), 4.41 (s, 2H), 4.25 (s, 2H), 3.74 (q, J = 4.4 Hz, 4H), 3.61 (s, 3H), 3.50 (q, J = 6.4 Hz, 1H), 3.43-3.36 (m, 2.5H), 3.15-3.05 (m, 4H), 2.85-2.72 (m, 1H), 2.18 (d, J = 12.4 Hz, 0.5H), 1.44- 1.12 (m, 9H), 0.95 (s, 1.5H), 0.81 (d, J = 6.4 Hz, 1.5H) ppm. MS: M/e 519 (M + 1)⁺. |
| A142 | 2-(6-((2S,5R)-2,5-dimethyl-4-(1- (3- (morpholinomethyl)phenyl)ethyl) piperazin-1-yl)-9-ethyl-3- methyl-2-oxo-3,9-dihydro-2H- purin-8-yl)acetonitrile | 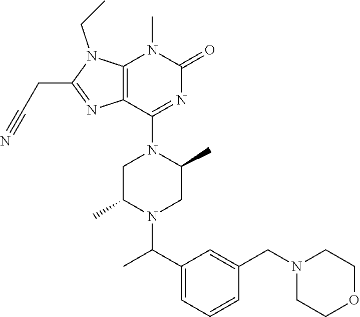 [SMILES: CCn1c(CC#N)nc2c(N3C[C@@H](C)N(C(C)c4cccc(CN5CCOCC5)c4)C[C@@H]3C)nc(=O)n(C)c21] | 58 mg | Compound A35 | ¹H NMR (400 MHz, DMSO-d₆): δ 7.35 (s, 1H), 7.23 (tdd, J = 21.1, 13.6, 7.3 Hz, 3H), 6.10-5.40 (m, 1H), 5.20-4.50 (m, 1H), 4.41 (s, 2H), 4.26 (q, J = 6.9 Hz, 2H), 3.70- 3.34 (m, 12H), 3.30-3.22 (m, 0.5H), 2.76 (s, 1H), 2.35 (s, 4H), 2.11 (d, J = 11.1 Hz, 0.5H), 1.49-1.11 (m, 9H), 1.01-0.73 (m, 3H) ppm. MS: M/e 533 (M + 1)⁺. |
| A143 | 2-(6-((2S,5R)-2,5-dimethyl-4-(1- (4- morpholinophenyl)ethyl)piperazin- 1-yl)-9-ethyl-3-methyl-2-oxo- 3,9-dihydro-2H-purin-8- yl)acetonitrile | 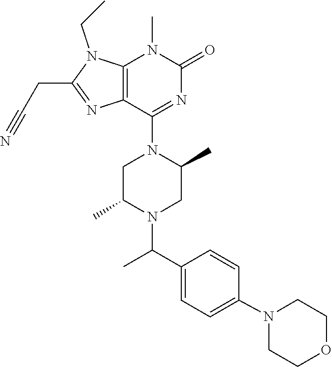 [SMILES: CCn1c(CC#N)nc2c(N3C[C@@H](C)N(C(C)c4ccc(N5CCOCC5)cc4)C[C@@H]3C)nc(=O)n(C)c21] | 17 mg | Compound A35 | ¹H NMR (400 MHz, DMSO-d₆) δ 7.24- 7.18 (m, 2H), 6.90 (t, J = 8.5 Hz, 2H), 6.15-5.40 (m, 1H), 5.23-4.42 (m, 1H), 4.40 (s, 2H), 4.26 (d, J = 7.6 Hz, 2H), 3.73 (s, 4H), 3.61 (s, 3H), 3.53-3.43 (m, 1H), 3.39- 3.35 (m, 0.5H), 3.32-3.29 (m, 2H), 3.12- 3.04 (m, 4H), 2.86-2.68 (m, 1H), 2.17 (d, J = 11.2 Hz, 0.5H), 1.34 (t, J = 7.0 Hz, 4H), 1.22 (d, J = 6.0 Hz, 3H), 1.16 (s, 2H), 0.93 (s, 2H), 0.80 (d, J = 5.9 Hz, 1H) ppm. MS: M/e 519 (M + 1)⁺. |
| A145 | 2-(6-((2S,5R)-2,5-dimethyl-4-(1- (p-tolyl)but-2-yn-1-yl)piperazin- 1-yl)-9-ethyl-3-methyl-2-oxo- 3,9-dihydro-2H-purin-8- yl)acetonitrile | 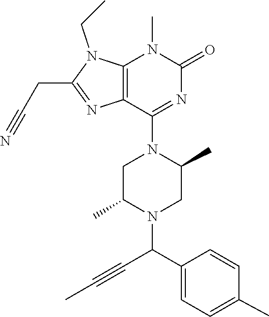 [SMILES: CC#CC(c1ccc(C)cc1)N1C[C@H](C)N(c2nc(=O)n(C)c3c2nc(CC#N)n3CC)C[C@H]1C] | 15 mg, 21% | Compound A35 | ¹H NMR (400 MHz, DMSO-d₆) δ 7.44 (d, J = 7.9 Hz, 1H), 7.37 (d, J = 7.9 Hz, 1H), 7.14 (d, J = 7.0 Hz, 2H), 5.73 (s, 1H), 5.19 (s, 1H), 4.37 (dd, J = 14.8, 7.6 Hz, 2H), 4.23 (s, 2H), 3.76 (s, 3H), 3.55 (d, J = 4.9 Hz, 1H), 3.18 (s, 1H), 3.02 (d, J = 8.9 Hz, 1H), 2.59 (m, 1H), 2.36 (m, 4H), 1.88 (dd, J = 7.7, 2.0 Hz, 3H), 1.46 (t, J = 7.1 Hz, 3H), 1.23 (dd, J = 21.3, 6.4 Hz, 3H), 1.09 (dd, J = 9.4, 6.7 Hz, 3H) ppm. MS: M/e 472 (M + 1)⁺. |
| A146 | 2-(6-((2S,5R)-4-(1-(5- (difluoromethyl)pyridin-2- yl)ethyl)-2,5-dimethyl piperazin- 1-yl)-3,9-dimethyl-2-oxo-3,9- dihydro-2H-purin-8- yl)acetonitrile | 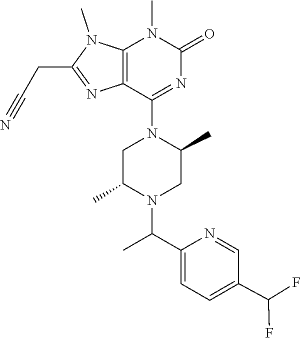 [SMILES: CC(c1ccc(C(F)F)cn1)N1C[C@H](C)N(c2nc(=O)n(C)c3c2nc(CC#N)n3C)C[C@H]1C] | A146a (3 mg) A146b (3 mg) | Compound A24 | Compound A146a (the earlier peak): ¹H NMR (400 MHz, CD₃OD) δ 8.63 (s, 1H), 8.02 (d, J = 8.0 Hz, 1H), 7.79 (d, J = 8.0 Hz, 1H), 6.91 (t, J = 55.6 Hz, 1H), 6.09- 4.99 (m, 2H), 4.26-4.16 (m, 1H), 3.93 (s, 3H), 3.79 (s, 3H), 3.77-3.72 (m, 1H), 3.67-3.58 (m, 1H), 3.33-3.29 (m, 2H), 2.91-2.74 (m, 1H), 2.13 (d, J = 12.0 Hz, 1H), 1.40 (d, J = 5.2 Hz, 3H), 1.30 (d, J = 3.2 Hz, 3H), 1.08 (d, J = 5.2 Hz, 3H) ppm. MS: M/e 471 (M + 1)⁺. Compound A146b (the later peak): ¹H NMR (400 MHz, CD₃OD) δ 8.64 (s, 1H), 8.02 (d, J = 8.0 Hz, 1H), 7.79 (d, J = 8.0 Hz, 1H), 6.91 (t, J = 55.6 Hz, 1H), 6.30-5.06 (m, 2H), 4.23-4.17 (m, 1H), 3.93 (s, 4H), 3.78 (s, 3H), 3.33-3.29 (m, 2H), 3.06-2.77 (m, 3H), 1.52-1.41 (m, 3H), 1.36 (d, J = 6.0 Hz, 3H), 0.96 (d, J = 6.0 Hz, 3H) ppm. MS: M/e 471 (M + 1)⁺. |
| A147 | 2-(6-((2S,5R)-2,5-dimethyl-4-(1- (5-methylpyridin-2- yl)ethyl)piperazin-1-yl)-3,9- dimethyl-2-oxo-3,9-dihydro-2H- purin-8-yl)acetonitrile | 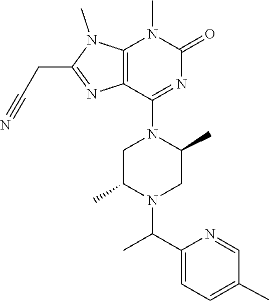 [SMILES: Cc1ccc(C(C)N2C[C@H](C)N(c3nc(=O)n(C)c4c3nc(CC#N)n4C)C[C@H]2C)nc1] | 8 mg | Compound A24 | ¹H NMR (400 MHz, CD₃OD) δ 8.35 (d, J = 7.6 Hz, 1H), 7.70 (d, J = 7.2 Hz, 1H), 7.51 (t, J = 9.2 Hz, 1H), 6.29-5.06 (m, 2H), 3.94 (s, 4H), 3.77 (s, 3.5H), 3.67- 3.40 (m, 1H), 3.33-3.29 (m, 2H), 3.08- 2.91 (m, 2H), 2.37 (s, 3H), 2.33-2.29 (m, 0.5H), 1.52-1.27 (m, 6H), 1.21-0.95 (m, 3H) ppm. MS: M/e 435 (M + 1)⁺. |
| A148 | 2-(6-((2S,5R)-2,5-dimethyl-4-(1- (5-(trifluoromethyl)pyridin-2- yl)ethyl)piperazin-1-yl)-3,9- dimethyl-2-oxo-3,9-dihydro-2H- purin-8-yl)acetonitrile | 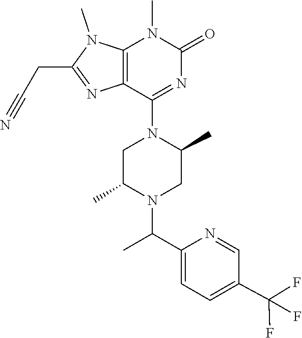 [SMILES: CC(c1ccc(C(F)(F)F)cn1)N1C[C@H](C)N(c2nc(=O)n(C)c3c2nc(CC#N)n3C)C[C@H]1C] | A148a (3 mg) A148b (2 mg) | Compound A24 | Compound A148a (the earlier peak): ¹H NMR (400 MHz, CD₃OD) δ 8.78 (s, 1H), 8.14 (d, J = 8.4 Hz, 1H), 7.87 (d, J = 8.4 Hz, 1H), 6.21-5.03 (m, 2H), 4.24-4.17 (m, 1H), 3.93 (s, 3H), 3.79 (s, 3H), 3.77- 3.71 (m, 1.5H), 3.65-3.57 (m, 1.5H), 3.33-3.29 (m, 1H), 2.89-2.74 (m, 1H), 2.11 (d, J = 12.4 Hz, 1H), 1.40 (d, J = 6.0 Hz, 3H), 1.30 (d, J = 4.2 Hz, 3H), 1.07 (d, J = 6.0 Hz, 3H) ppm. MS: M/e 489 (M + 1)⁺. Compound A148b (the later peak): ¹H NMR (400 MHz, CD₃OD) δ 8.79 (s, 1H), 8.14 (d, J = 8.0 Hz, 1H), 7.86 (d, J = 8.0 Hz, 1H), 6.33-5.05 (m, 2H), 4.24-4.16 (m, 1H), 3.99-3.95 (m, 1H), 3.93 (s, 3H), 3.78 (s, 3H), 3.33-3.29 (m, 2H), 3.04- 2.76 (m, 3H), 1.45 (d, J = 4.2 Hz, 3H), 1.36 (d, J = 6.4 Hz, 3H), 0.96 (d, J = 6.4 Hz, 3H) ppm. MS: M/e 489 (M + 1)⁺. |
| A151 | 2-(6-((2S,5R)-2,5-diethyl-4-(1- (4-(trifluoromethyl)phenyl)ethyl) piperazin-1-yl)-3-methyl-2-oxo- 3,9-dihydro-2H-purin-8- yl)acetonitrile | 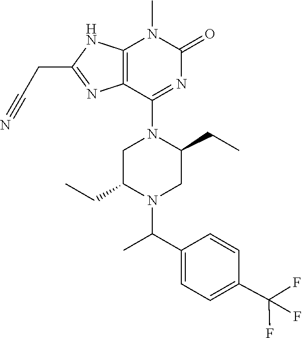 [SMILES: CC[C@H]1CN(C(C)c2ccc(C(F)(F)F)cc2)[C@H](CC)CN1c1nc(=O)n(C)c2[nH]c(CC#N)nc12] | A15la (3 mg) A151b (10 mg) | Compound A150 | Compound A15la (the earlier peak): ¹H NMR (400 MHz, CD₃OD) δ 7.70-7.53 (m, 4H), 6.02-5.07 (m, 2H), 3.95-3.88 (m, 3H), 3.85-3.79 (m, 1H), 3.78 (s, 3H), 3.49-3.37 (m, 1H), 3.06-2.96 (m, 1H), 2.40-2.29 (m, 1H), 2.12-1.91 (m, 2H), 1.57-1.42 (m, 2H), 1.33 (d, J = 6.4 Hz, 3H), 0.93 (t, J = 7.2 Hz, 3H), 0.73-0.63 (m, 3H) ppm. MS: M/e 502 (M + 1)⁺. Compound A151b (the later peak): ¹H NMR (400 MHz, CD₃OD) δ 7.66-7.54 (m, 4H), 6.21-5.08 (m, 2H), 4.27-4.14 (m, 1H), 3.94-3.89 (m, 2H), 3.78 (s, 3H), 3.68-3.60 (m, 1H), 3.24-3.12 (m, 1H), 2.67-2.51 (m, 1H), 2.30-2.21 (m, 1H), 1.90-1.71 (m, 2H), 1.57-1.44 (m, 2H), 1.31 (d, J = 6.4 Hz, 3H), 1.06-0.97 (m, 3H), 0.66 (t, J = 7.2 Hz, 3H) ppm. MS: M/e 502 (M + 1)⁺. |
| A154 | 2-(6-((2S,5R)-4-(1-(isoquinolin- 3-yl)ethyl)-2,5- dimethylpiperazin-1-yl)-3,9- dimethyl-2-oxo-3,9-dihydro-2H- purin-8-yl)acetonitrile | 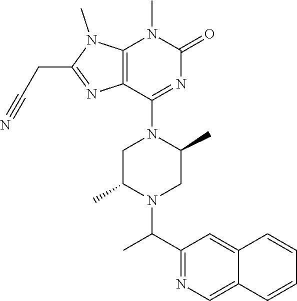 [SMILES: CC(c1cc2ccccc2cn1)N1C[C@H](C)N(c2nc(=O)n(C)c3c2nc(CC#N)n3C)C[C@H]1C] | 15 mg | Compound A24 | ¹H NMR (400 MHz, CD₃OD) δ 9.23 (d, J = 3.6 Hz, 1H), 8.10 (d, J = 8.2 Hz, 1H), 7.93 (t, J = 10.1 Hz, 2H), 7.78 (t, J = 7.5 Hz, 1H), 7.66 (t, J = 7.5 Hz, 1H), 6.18- 4.87 (m, 2H), 4.20 (s, 1H), 4.03 (q, J = 6.4 Hz, 1H), 3.94 (s, 3H), 3.78 (s, 3H), 3.74- 3.38 (m, 2H), 3.10-2.23 (m, 3H), 1.60- 1.24 (m, 6H), 1.07 (dd, J = 59.0, 6.4 Hz, 3H) ppm. MS: M/e 471 (M + 1)⁺. |
| A155 | 2-(6-((2S,5R)-4-(1-(1,2- dimethyl-4-oxo-1,4- dihydroquinolin-7-yl)ethyl)-2,5- dimethylpiperazin-1-yl)-9-ethyl- 3-methyl-2-oxo-3,9-dihydro-2H- purin-8-yl)acetonitrile | 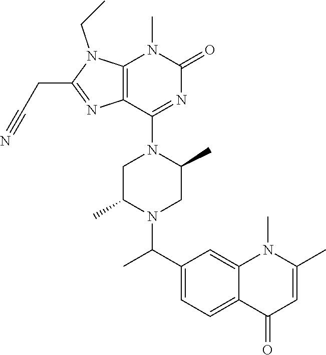 [SMILES: CCn1c(CC#N)nc2c(N3C[C@@H](C)N(C(C)c4ccc5c(=O)cc(C)n(C)c5c4)C[C@@H]3C)nc(=O)n(C)c21] | 25 mg | Compound A35 | ¹H NMR (400 MHz, CD₃OD) δ 7.92- 7.78 (m, 1H), 7.64 (d, J = 14.9 Hz, 1H), 7.45 (dd, J = 16.6, 8.1 Hz, 1H), 6.56 (s, 1H), 6.20-4.99 (m, 2H), 4.37 (d, J = 6.9 Hz, 2H), 4.22 (s, 2H), 3.83 (s, 1H), 3.81- 3.46 (m, 8H), 2.92 (s, 1H), 2.75-2.60 (m, 0.5H), 2.53 (s, 3H), 2.30-2.10 (m, 0.5H), 1.54-1.25 (m, 9H), 1.02 (dd, J = 57.1, 5.3 Hz, 3H) ppm. MS: M/e 529 (M + 1)⁺. |
| A160 | 2-(6-((2S,5R)-4-(1-(7-chloro- 3,3-dimethyl-2,3-dihydro- [1,4]dioxino[2,3-b]pyridin-6- yl)ethyl)-2,5-dimethylpiperazin- 1-yl)-9-ethyl-3-methyl-2-oxo- 3,9-dihydro-2H-purin-8- yl)acetonitrile | 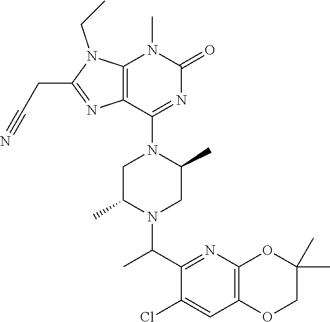 [SMILES: CCn1c(CC#N)nc2c(N3C[C@@H](C)N(C(C)c4nc5c(cc4Cl)OCC(C)(C)O5)C[C@@H]3C)nc(=O)n(C)c21] | 14 mg | Compound A35 | ¹H NMR (400 MHz, CD₃OD) δ 7.36 (s, 1H), 6.15-4.99 (m, 1H), 4.85-4.81 (m, 1H), 4.37 (d, J = 6.7 Hz, 2H), 4.23 (s, 2H), 4.00 (s, 2H), 3.76 (s, 3H), 3.73-3.36 (m, 1H), 3.31-2.62 (m, 3H), 2.30 (s, 1H), 1.52-1.22 (m, 15H), 1.19-0.96 (m, 3H) ppm. MS: M/e 555 (M + 1)⁺. |
| A170 | 2-(6-((2S,5R)-4-(1-(6-chloro- 2,2- dimethylbenzo[d][1,3]dioxol-5- yl)ethyl)-2,5-dimethylpiperazin- 1-yl)-9-ethyl-3-methyl-2-oxo- 3,9-dihydro-2H-purin-8- yl)acetonitrile | 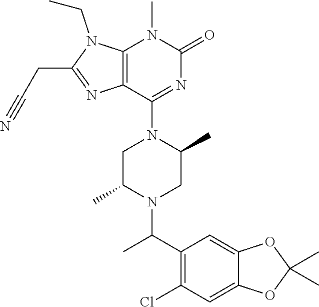 [SMILES: CCn1c(CC#N)nc2c(N3C[C@@H](C)N(C(C)c4cc5c(cc4Cl)OC(C)(C)O5)C[C@@H]3C)nc(=O)n(C)c21] | 37 mg, 23% | Compound A35 | ¹H NMR (400 MHz, CD₃OD) δ 7.08 (s, 0.5H), 7.03 (s, 0.5H), 6.73 (s, 0.5H), 6.71 (s, 0.5H), 6.30-5.51 (m, 1H), 5.30-5.01 (m, 1H), 4.45-4.30 (m, 2H), 4.28-4.16 (m, 1H), 4.11-3.90 (m, 1H), 3.85-3.73 (s, 3.5H), 3.65-3.50 (m, 1H), 3.35-3.31 (m, 1H), 3.01-2.60 (m, 2H), 2.24-2.14 (m, 0.5H), 1.75-1.60 (m, 6H), 1.52-1.19 (m, 9H), 1.10-0.85 (m, 3H) ppm. MS: M/e 540 (M + 1)⁺. |
| A171 | 2-(6-((2S,5R)-4-(1-(7-chloro- 2,2- dimethylbenzo[d][1,3]dioxol-5- yl)ethyl)-2,5-dimethylpiperazin- 1-yl)-9-ethyl-3-methyl-2-oxo- 3,9-dihydro-2H-purin-8- yl)acetonitrile | 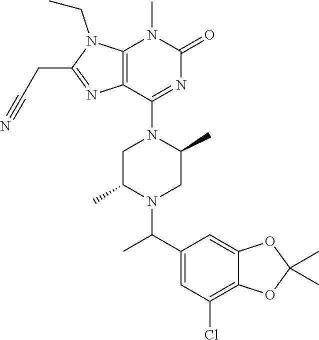 [SMILES: CCn1c(CC#N)nc2c(N3C[C@@H](C)N(C(C)c4cc(Cl)c5c(c4)OC(C)(C)O5)C[C@@H]3C)nc(=O)n(C)c21] | 5 mg, 10% | Compound A35 | ¹H NMR (400 MHz, CD₃OD) δ 6.86- 6.72 (m, 2H), 6.23-5.75 (m, 1H), 5.31- 4.95 (m, 1H), 4.44-4.31 (m, 2H), 4.26- 4.17 (m, 1H), 3.76 (s, 3H), 3.62-3.44 (m, 2H), 3.35-3.31 (m, 1H), 3.01-2.57 (m, 2.5H), 2.30-2.16 (m, 0.5H), 1.69 (s, 6H), 1.54-1.39 (m, 4.5H), 1.36-1.22 (m, 4.5H), 1.02 (d, J = 4.8 Hz, 1.5H), 0.91 (d, J = 5.6 Hz, 1.5H) ppm. MS: M/e 540 (M + 1)⁺. |
| A177 | 2-(6-((2S,5R)-2,5-dimethyl-4-(1- (2,2,7- trimethylbenzo[d][1,3]dioxol-5- yl)ethyl)piperazin-1-yl)-9-ethyl- 3-methyl-2-oxo-3,9-dihydro-2H- purin-8-yl)acetonitrile | 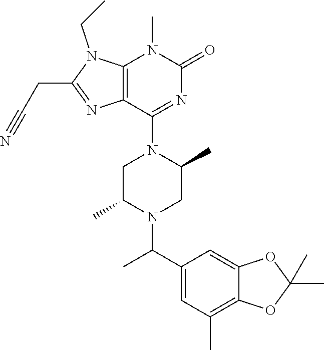 [SMILES: CCn1c(CC#N)nc2c(N3C[C@@H](C)N(C(C)c4cc(C)c5c(c4)OC(C)(C)O5)C[C@@H]3C)nc(=O)n(C)c21] | 52 mg, 33% | Compound A35 | ¹H NMR (400 MHz, CD₃OD) δ 6.67 (s, 1H), 6.62 (s, 1H), 6.40-4.90 (m, 2H), 4.45-4.30 (m, 2H), 4.23 (s, 1H), 3.76 (s, 3H), 3.68-3.39 (m, 2H), 3.21-2.26 (m, 4H), 2.15 (d, J = 6.0 Hz, 3H), 1.64 (s, 6H), 1.52-1.20 (m, 9H), 1.13-0.89 (m, 3H) ppm. MS: M/e 520 (M + 1)⁺. |
| A180 | 2-(6-((2S,5R)-4-(1-(2,2- dimethyl-2,3-dihydrofuro[2,3- b]pyridin-6-yl)ethyl)-2,5- dimethylpiperazin-1-yl)-9-ethyl- 3-methyl-2-oxo-3,9-dihydro-2H- purin-8-yl)acetonitrile | 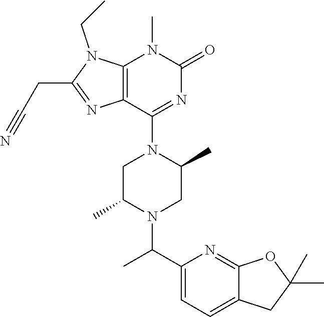 [SMILES: CCn1c(CC#N)nc2c(N3C[C@@H](C)N(C(C)c4ccc5c(n4)OC(C)(C)C5)C[C@@H]3C)nc(=O)n(C)c21] | 27 mg | Compound A35 | ¹H NMR (400 MHz, DMSO-d₆) δ 7.30 (t, J = 6.8 Hz, 1H), 6.24-6.16 (m, 1H), 6.01- 5.31 (m, 1H), 5.23-4.50 (m, 1H), 4.40 (s, 2H), 4.26 (q, J = 6.6 Hz, 2H), 3.70-3.51 (m, 4.5H), 3.50-3.35 (m, 3.5H), 3.02 (s, 0.5H), 2.80-2.60 (m, 1H), 2.17 (d, J = 11.3 Hz, 0.5H), 1.86 (s, 3H), 1.79 (s, 3H), 1.37- 1.20 (m, 9H), 0.90 (s, 3H) ppm. MS: M/e 505 (M + 1)⁺. |
| A182 | 2-(6-((2S,5R)-4-(3,3-dimethyl- 2,3-dihydrofuro[3,2-b]pyridine- 5-carbonyl)-2,5- dimethylpiperazin-1-yl)-9-ethyl- 3-methyl-2-oxo-3,9-dihydro-2H- purin-8-yl)acetonitrile | 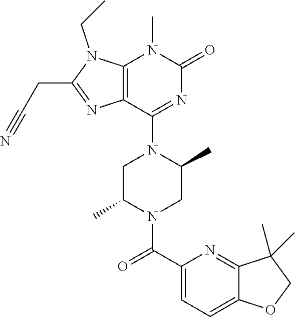 [SMILES: CCn1c(CC#N)nc2c(N3C[C@@H](C)N(C(=O)c4ccc5c(n4)C(C)(C)CO5)C[C@@H]3C)nc(=O)n(C)c21] | 7 mg | Compound A35 and A181 | ¹H NMR (400 MHz, CD₃OD) δ 7.44 (dd, J = 19.8, 7.9 Hz, 1H), 7.21 (dd, J = 8.3, 5.6 Hz, 1H), 6.51-4.89 (m, 2H), 4.45 (d, J = 3.3 Hz, 2H), 4.44-4.18 (m, 4H), 3.94- 3.80 (m, 1H), 3.78 (s, 3H), 3.72-3.36 (m, 2H), 2.65 (s, 1H), 1.52-1.44 (m, 3H), 1.43-1.37 (m, 9H), 1.35-1.28 (m, 3H) ppm. MS: M/e 505 (M + 1)⁺. |
| A186 | 2-(6-((2S,5R)-4-(1-(2,2- dimethylbenzo[d][1,3]dioxol-4- yl)ethyl)-2,5-dimethylpiperazin- 1-yl)-9-ethyl-3-methyl-2-oxo- 3,9-dihydro-2H-purin-8- yl)acetonitrile | 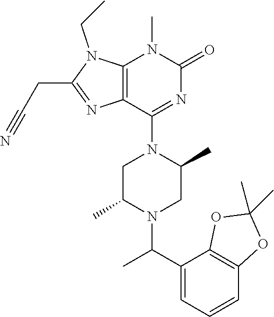 [SMILES: CCn1c(CC#N)nc2c(N3C[C@@H](C)N(C(C)c4cccc5c4OC(C)(C)O5)C[C@@H]3C)nc(=O)n(C)c21] | 42 mg, 28% | Compound A35 | ¹H NMR (400 MHz, CD₃OD) δ 6.97- 6.86 (m, 1H), 6.84-6.72 (m, 1H), 6.67- 6.57 m, 1H), 6.20-5.40 (m, 1H), 5.36- 4.60 (m, 1H), 4.45-4.31 (m, 2H), 4.23 (s, 2H), 3.95-3.51 (m, 5H), 3.08-2.91 (m, 1H), 2.87-2.66 (m, 1H), 2.45-2.30 (m, 1H), 1.70-1.60 (m, 6H), 1.50-1.20 (m, 9H), 1.10-0.91 (m, 3H) ppm. MS: M/e 506 (M + 1)⁺. |
| A187 | 2-(6-((2S,5R)-4-(1-(2,2- difluorobenzo[d][1,3]dioxol-4- yl)ethyl)-2,5-dimethylpiperazin- 1-yl)-9-ethyl-3-methyl-2-oxo- 3,9-dihydro-2H-purin-8- yl)acetonitrile | 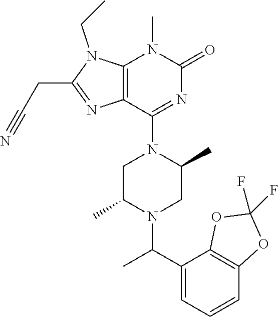 [SMILES: CCn1c(CC#N)nc2c(N3C[C@@H](C)N(C(C)c4cccc5c4OC(F)(F)O5)C[C@@H]3C)nc(=O)n(C)c21] | 55 mg, 36% | Compound A35 | ¹H NMR (400 MHz, CD₃OD) δ 7.33- 6.98 (m, 3H), 6.20-5.50 (m, 2H), 4.44- 4.30 (m, 2H), 4.23 (s, 2H), 3.97-3.50 (m, 6H), 2.98-2.15 (m, 2H), 1.51-1.20 (m, 9H), 1.10-0.90 (m, 3H) ppm. MS: M/e 514 (M + 1)⁺. |
| A192 | 2-(6-((2S,5R)-2,5-dimethyl-4-(1- (3-methyl-4-oxo-3,4-dihydro- 2H-benzo[e][1,3]oxazin-6- yl)ethyl)piperazin-1-yl)-9-ethyl- 3-methyl-2-oxo-3,9-dihydro-2H- purin-8-yl)acetonitrile | 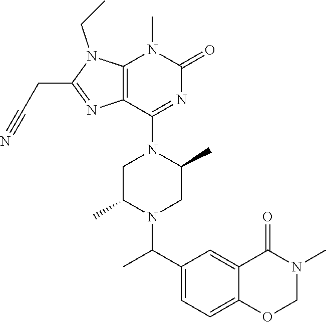 [SMILES: CCn1c(CC#N)nc2c(N3C[C@@H](C)N(C(C)c4ccc5c(c4)C(=O)N(C)CO5)C[C@@H]3C)nc(=O)n(C)c21] | 63 mg | Compound A35 | ¹H NMR (400 MHz, DMSO-d₆) δ 7.79 (d, J = 13.6 Hz, 1H), 7.52 (dd, J = 15.4, 8.4 Hz, 1H), 7.03 (t, J = 9.5 Hz, 1H), 6.12- 5.42 (m, 1H), 5.28 (d, J = 3.4 Hz, 2H), 5.15-4.50 (m, 1H), 4.41 (s, 2H), 4.25 (s, 2H), 3.61 (s, 4H), 3.55-3.30 (m, 2H), 2.98 (s, 3H), 2.76 (s, 1.5H), 2.10 (d, J = 12.6 Hz, 0.5H), 1.34 (t, J = 5.8 Hz, 4.5H), 1.25 (t, J = 5.8 Hz, 3H), 1.16 (s, 1.5H), 0.96 (s, 1.5H), 0.82 (d, J = 5.5 Hz, 1.5H) ppm. MS: M/e 519 (M + 1)⁺. |
| A193 | 2-(6-((2S,5R)-4-(1-(1,3- dimethyl-4-oxo-1,4- dihydroquinolin-6-yl)ethyl)-2,5- dimethylpiperazin-1-yl)-9-ethyl- 3-methyl-2-oxo-3,4,5,9- tetrahydro-2H-purin-8- yl)acetonitrile | 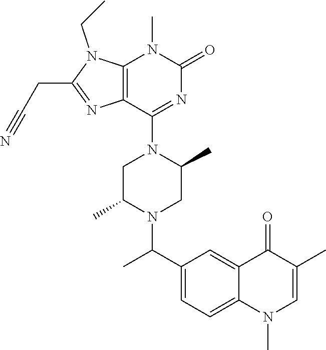 [SMILES: CCn1c(CC#N)nc2c(N3C[C@@H](C)N(C(C)c4ccc5c(c4)c(=O)c(C)cn5C)C[C@@H]3C)nc(=O)n(C)c21] | 6 mg | Compound A35 | ¹H NMR (400 MHz, CD₃OD) δ 8.34 (s, 1H), 8.00 (d, J = 5.5 Hz, 1H), 7.91 (d, J = 8.4 Hz, 1H), 7.73 (t, J = 9.9 Hz, 1H), 6.50- 5.00 (m, 2H), 4.37 (d, J = 6.4 Hz, 2H), 4.22 (s, 2H), 3.95 (s, 3H), 3.88 (s, 1H), 3.76 (s, 3H), 3.73-3.43 (m, 1H), 3.07- 2.19 (m, 3H), 2.15 (s, 3H), 1.55-1.20 (m, 9H), 1.03 (d, J = 62.6 Hz, 3H) ppm. MS: M/e 529 (M + 1)⁺. |
| A196 | 2-(7-((2S,5R)-2,5-dimethyl-4-(1- (4-methyl-2- (trifluoromethyl)phenyl) ethyl)piperazin-1-yl)-4-methyl- 5-oxo-4,5-dihydrothiazolo[5,4- b]pyridin-2-yl)acetonitrile | 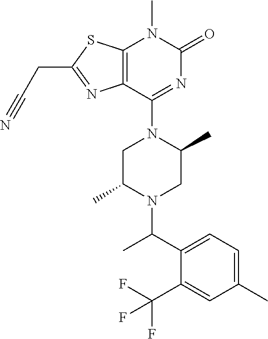 [SMILES: Cc1ccc(C(C)N2C[C@H](C)N(c3nc(=O)n(C)c4sc(CC#N)nc34)C[C@H]2C)c(C(F)(F)F)c1] | 2 mg, 8% | Compound A194 and A195 | ¹H NMR (400 MHz, CDCl₃) δ 7.77 (dd, J = 19.5, 7.9 Hz, 1H), 7.40-7.24 (m, 2H), 5.65 (s, 1H), 4.01 (s, 2H), 3.98-3.84 (m, 1H), 3.52 (s, 4H), 3.49-3.24 (m, 1H), 2.98-2.69 (m, 2H), 2.41-2.25 (m, 4H), 1.98-1.87 (m, 1H), 1.35 (d, J = 6.6 Hz, 2H), 1.22 (d, J = 6.3 Hz, 2H), 1.15 (s, 1H), 1.08 (t, J = 5.8 Hz, 2H), 0.93 (d, J = 6.4 Hz, 2H) ppm. MS: m/e 504 (M + 1)⁺. |

### Assay

### Biochemical DGK IC50 Assays

Enzymatic reactions of DGKζ, DGKα and DGKδ were performed using ADP-Glo assay with lipid micelle substrate. Full length DGKζ (in-house protein M1-V929 with SEQ ID No: 2) was expressed in baculovirus expression system. Full length DGKα (D21-10BG, SignalChem) and full length DGKδ (D23-10G, SignalChem) were purchased. Lipid micelle was prepared by dissolving DAG (Sigma, 317505-10MG) and PS (Sigma, P7769-100MG) with chloroform which was furtherly removed by rotary evaporation. The obtained residue was resuspended in buffer containing 25 mM HEPES pH 7.0, 0.5 mM EDTA and 160 mM Octyl β-D glucopyranoside by vigorous mixing and ultrasonic (IID, Scientz).

Final concentration in reaction

| Enzyme | Enzyme (nM) | ATP (μM) | DAG (μM) |
|---|---|---|---|
| DGKα | 7.5 | 140 | 80 |
| DGKδ | 10 | 140 | 80 |
| DGKζ | 0.5 | 140 | 80 |

The inhibition activities testing for the compound disclosed herein were carried out at room temperature in assay buffer containing 50 mM HEPES, 10 mM MgCl₂, 0.01% BSA, 0.1 mM Na₃VO₄, 0.005% Tween-20 and 0.01 mM CaCl₂. Compounds in DMSO were dispensed into wells of a black 384 well plate (Corning 4514) using D300e digital dispenser (Tecan). The ranges of compounds final concentration were 1.55˜10000 nM or 23.3˜150000 nM. 3 μL 2× enzyme solution was added to wells. After incubation for 1 hour, 3 μL 2× substates solution containing 160 μM DAG and 280 μM ATP was added to the wells to initiate reaction. After 1 hour reaction, 5 μL ADP-Glo reagent (Promga V9101) was added and incubated for 40 minutes. 10 μL Kinase Detection reagent was added and incubated for 30 minutes. Luminescence was measured on a microplate reader (PHERAstar FSX, BMG labtech). The IC₅₀s are calculated based on inhibition of enzyme activity in the presence of increasing concentrations of compounds. Selected compounds had no inhibitory activity on DGKδ. IC₅₀s of the compounds disclosed herein for DGKζ and DGKα are shown in Table 1.

### Baculovirus Expression of Human DGKζ

Human His-TEV-DGK-zeta-pFastBacl and human baculovirus samples was generated using the Bac-to-Bac baculovirus expression system (Invitrogen) according to the manufacturer's protocol. The DNA used for expression of DGK-zeta, have SEQ ID Nos: 1. Baculovirus amplification was achieved using infected SF9 cells at 1:2000 virus/cells ratios, and grown for 96 hours at 27° C. post-transfected.

The expression scaled up for each protein was carried out in the flask 3 L from CORNING. 4 L of 3×10⁶ cells/mL Sf9 cells (Expression System, Invitrogen) grown in SF900™ II SFM insect medium (Expression System) was infected with virus stock at 1:200 virus/cells ratio, and grown for 48 hours at 27° C. post-transfection. The infected cell culture was harvested by centrifugation at 6000 rpm for 15 min 4° C. in a SORVALL LYNX6000 centrifuge. The cell pellets were stored at −80° C.

### Purification of Human DGK-zeta

Full length human DGKζ produced as described above, was purified from Sf9 baculovirus-infected insect cell paste. The cells were lysed using sonication method, and the lysates were clarified by centrifugation. The clarified lysates were purified to ˜90% homogeneity, using two successive column chromatography steps on an AKTA Purifier system. The two steps column chromatography included nickel affinity resin capture (i.e. Ni-NTA Agarose, Qiagen), followed by size exclusion chromatography (i.e. Hiload16/60 Superdex200 prep grade, GE Healthcare. The protein was delivered and stored at −80° C. The formulation buffers were identical for the protein: 25 mM Tris, 150 mmol/L NaCl, 2 mM DTT, pH8.0.

SEQ ID No: 1 is Nucleotide sequence encoding His-TEV-hDGKζ-(M1-V929):
ATGCATCACCATCACCATCACCATCACGAGAACCTGTACTTCCAGGGATCCATGGA
ACCCCGTGATGGTAGCCCCGAAGCTCGTAGCTCCGATTCCGAGTCCGCCAGCGCTTCCTCCT
CCGGTAGCGAACGTGACGCTGGTCCCGAGCCCGACAAAGCTCCCCGTCGTCTGAATAAGCG
CCGTTTTCCCGGTCTCCGTCTGTTCGGCCACCGCAAGGCCATCACTAAGTCCGGTCTCCAGCA
TCTGGCTCCTCCTCCTCCTACCCCCGGTGCTCCTTGCTCCGAATCCGAGCGCCAGATTCGCTC
CACTGTGGATTGGTCCGAAAGCGCCACCTATGGTGAGCATATCTGGTTCGAGACCAACGTCT
CCGGCGACTTCTGTTATGTCGGTGAGCAATACTGTGTGGCTCGTATGCTGCAGAAGTCCGTG
TCCCGCCGTAAATGCGCCGCTTGCAAAATCGTGGTCCATACCCCTTGCATCGAGCAACTGGA
GAAAATCAACTTCCGCTGCAAGCCCAGCTTTCGTGAGTCCGGTTCCCGCAACGTGCGCGAAC
CTACTTTCGTGCGCCACCACTGGGTGCATCGTCGTCGCCAAGACGGCAAATGCCGCCACTGC
GGCAAAGGTTTTCAGCAGAAATTCACCTTCCACAGCAAGGAGATCGTCGCCATCAGCTGCAG
CTGGTGCAAACAAGCTTACCATTCCAAAGTGAGCTGCTTCATGCTCCAGCAGATCGAAGAGC
CTTGCTCTCTGGGTGTGCATGCTGCTGTCGTGATTCCCCCTACTTGGATTCTGCGTGCTCGCC
GTCCCCAGAACACTCTGAAGGCCTCCAAAAAGAAGAAGCGCGCCAGCTTCAAGCGTAAGAG
CTCCAAAAAGGGTCCCGAAGAGGGCCGTTGGCGTCCCTTCATCATCCGCCCTACTCCTTCCC
CTCTGATGAAGCCTCTGCTGGTCTTCGTCAACCCTAAGAGCGGCGGCAACCAAGGTGCTAAA
ATCATCCAGTCCTTCCTCTGGTATCTGAACCCTCGTCAAGTGTTCGACCTCAGCCAAGGCGGT
CCTAAGGAGGCTCTGGAGATGTACCGCAAGGTCCACAATCTGCGCATCCTCGCTTGTGGTGG
CGATGGCACCGTGGGCTGGATTCTGTCCACTCTGGACCAACTGCGTCTGAAACCTCCCCCCC
CCGTGGCTATTCTGCCTCTCGGTACCGGCAACGATCTGGCTCGTACTCTGAATTGGGGTGGT
GGCTACACCGATGAGCCCGTGTCCAAGATTCTGTCCCACGTCGAAGAAGGCAATGTCGTCCA
ACTGGACCGTTGGGACCTCCACGCCGAACCCAACCCCGAGGCTGGCCCCGAGGACCGTGAC
GAGGGCGCTACTGACCGTCTGCCCCTCGACGTCTTCAATAATTACTTCTCTCTGGGCTTTGAC
GCTCACGTGACTCTGGAATTTCATGAAAGCCGCGAGGCCAACCCCGAGAAGTTCAATTCCCG
TTTCCGCAACAAGATGTTCTACGCTGGCACCGCCTTCAGCGACTTCCTCATGGGCTCCAGCA
AGGACCTCGCTAAGCATATCCGCGTGGTGTGCGATGGCATGGATCTGACCCCTAAGATCCAA
GATCTGAAGCCCCAATGTGTCGTGTTTCTGAACATCCCCCGCTACTGCGCTGGTACTATGCCT
TGGGGCCATCCCGGTGAACACCATGACTTCGAACCTCAGCGTCATGATGACGGCTATCTGGA
GGTGATCGGTTTCACCATGACCTCCCTCGCTGCTCTGCAAGTGGGTGGCCACGGCGAACGTC
TGACTCAATGCCGCGAGGTGGTGCTGACCACCAGCAAAGCCATCCCCGTCCAAGTGGATGGT
GAGCCTTGCAAGCTGGCCGCCTCCCGTATCCGTATCGCTCTCCGCAATCAAGCTACCATGGT
CCAGAAGGCCAAACGCCGCAGCGCTGCTCCTCTCCACAGCGACCAACAACCCGTCCCCGAA
CAGCTGCGCATCCAAGTGTCCCGTGTCAGCATGCATGACTACGAGGCTCTGCACTACGACAA
GGAACAGCTGAAGGAAGCCAGCGTGCCTCTGGGTACTGTGGTCGTGCCCGGTGACAGCGAT
CTGGAGCTCTGCCGTGCCCACATCGAGCGTCTGCAGCAAGAGCCCGACGGTGCTGGTGCCAA
GAGCCCTACTTGCCAAAAACTCTCCCCCAAGTGGTGTTTCCTCGACGCTACCACCGCCAGCC
GCTTCTACCGCATTGATCGCGCCCAAGAGCATCTGAACTATGTCACCGAGATCGCTCAAGAC
GAGATCTACATCCTCGACCCCGAACTCCTCGGTGCTAGCGCCCGTCCCGACCTCCCCACTCCT
ACCTCCCCTCTGCCCACTTCCCCTTGTTCCCCCACCCCTCGTAGCCTCCAAGGTGATGCTGCC
CCTCCTCAAGGTGAGGAGCTCATTGAGGCCGCTAAGCGTAACGATTTCTGCAAGCTCCAAGA
GCTGCATCGTGCTGGTGGCGACCTCATGCACCGCGATGAGCAGAGCCGCACTCTGCTGCACC
ACGCTGTGTCCACTGGTAGCAAGGACGTGGTGCGCTATCTGCTGGACCACGCTCCTCCCGAG
ATCCTCGACGCTGTGGAAGAAAACGGCGAGACTTGCCTCCACCAAGCTGCTGCTCTGGGTCA
ACGTACCATCTGCCACTACATCGTCGAAGCTGGTGCTTCTCTGATGAAGACCGACCAGCAAG
GTGATACTCCCCGTCAGCGCGCCGAGAAAGCCCAAGACACCGAACTGGCTGCCTATCTGGA
GAACCGTCAGCACTACCAGATGATTCAGCGTGAAGACCAAGAGACCGCCGTGTAA
SEQ ID No: 2 is Amino acid sequence of His-TEV-hDGKζ-(M1-V929):
MEPRDGSPEARSSDSESASASSSGSERDAGPEPDKAPRRLNKRRFPGLRLFGHRKAITKS
GLQHLAPPPPTPGAPCSESERQIRSTVDWSESATYGEHIWFETNVSGDFCYVGEQYCVARMLQKS
VSRRKCAACKIVVHTPCIEQLEKINFRCKPSFRESGSRNVREPTFVRHHWVHRRRQDGKCRHCG
KGFQQKFTFHSKEIVAISCSWCKQAYHSKVSCFMLQQIEEPCSLGVHAAVVIPPTWILRARRPQN
TLKASKKKKRASFKRKSSKKGPEEGRWRPFIIRPTPSPLMKPLLVFVNPKSGGNQGAKIIQSFLWY
LNPRQVFDLSQGGPKEALEMYRKVHNLRILACGGDGTVGWILSTLDQLRLKPPPPVAILPLGTGN
DLARTLNWGGGYTDEPVSKILSHVEEGNVVQLDRWDLHAEPNPEAGPEDRDEGATDRLPLDVF
NNYFSLGFDAHVTLEFHESREANPEKFNSRFRNKMFYAGTAFSDFLMGSSKDLAKHIRVVCDGM
DLTPKIQDLKPQCVVFLNIPRYCAGTMPWGHPGEHHDFEPQRHDDGYLEVIGFTMTSLAALQVG
GHGERLTQCREVVLTTSKAIPVQVDGEPCKLAASRIRIALRNQATMVQKAKRRSAAPLHSDQQP
VPEQLRIQVSRVSMHDYEALHYDKEQLKEASVPLGTVVVPGDSDLELCRAHIERLQQEPDGAGA
KSPTCQKLSPKWCFLDATTASRFYRIDRAQEHLNYVTEIAQDEIYILDPELLGASARPDLPTPTSPL
PTSPCSPTPRSLQGDAAPPQGEELIEAAKRNDFCKLQELHRAGGDLMHRDEQSRTLLHHAVSTGS
KDVVRYLLDHAPPEILDAVEENGETCLHQAAALGQRTICHYIVEAGASLMKTDQQGDTPRQRAE
KAQDTELAAYLENRQHYQMIQREDQETAV*

TABLE 1

| Compound | DGKζ ADP-Glo | DGKα ADP-Glo |
|---|---|---|
| No. | assay IC50(nM) | assay IC50(nM) |
| A1 | 19 | 20,200 |
| A1a | 9 | 14,900 |
| A1b | 2,350 | >10,000 |
| A2 | 92 | >150,000 |
| A3 | 398 | 822 |
| A4a | 566 | >10,000 |
| A4b | 64 | >10,000 |
| A5a | 878 | 5,770 |
| A5b | 88 | 616 |
| A6a | 528 | >10,000 |
| A6b | 13 | >10,000 |
| A7a | 3,110 | >10,000 |
| A7b | 35 | 97,600 |
| A8a | 1,480 | >10,000 |
| A8b | 97 | >150,000 |
| A9 | 262 | >10,000 |
| A9a | 8,590 | >10,000 |
| A9b | 169 | >10,000 |
| A10 | 25 | >10,000 |
| A10a | 405 | >10,000 |
| A10b | 14 | >10,000 |
| A11a | 764 | 57,800 |
| A11b | 14 | 11,900 |
| A12a | 2.7 | 18,800 |
| A12b | 57 | 27,400 |
| A13a | 5,060 | >150,000 |
| A13b | 54 | >150,000 |
| 14a | 1,420 | >10,000 |
| A14b | 180 | >10,000 |
| A15a | 1,410 | >10,000 |
| A15b | 291 | >10,000 |
| A16 | 24 | 58,200 |
| A17a | 133 | 1,460 |
| A17b | 409 | 1,580 |
| A18a | 19 | 23,600 |
| A18b | 327 | 6,380 |
| A19a | 2.8 | 27,500 |
| A19b | 82 | 6,940 |
| A20 | 5 | 93,000 |
| A21 | 61 | >150,000 |
| A22a | 26 | >10,000 |
| A23a | 8 | 63,200 |
| A23b | 94 | 16,200 |
| A24a | 975 | >10,000 |
| A24b | 42 | 131,000 |
| A25 | 15 | 36,900 |
| A26 | 74 | 4,240 |
| A27a | 569 | >10,000 |
| A27b | 15 | 99,500 |
| A28 | 3 | 96,900 |
| A29a | 437 | >150,000 |
| A29b | 4 | 136,000 |
| A30 | 14 | 44,300 |
| A31 | 4 | 110,000 |
| A32a | 3,740 | >10,000 |
| A32b | 138 | 5,590 |
| A33a | 804 | >10,000 |
| A33b | 3 | >10,000 |
| A34a | 392 | >10,000 |
| A34b | 9 | >10,000 |
| A35a | 2,760 | >150,000 |
| A35b | 24 | >150,000 |
| A36 | 17 | 104,000 |
| A37 | 18 | 128,000 |
| A38 | 165 | 143,000 |
| A39 | 211 | >150,000 |
| A40 | 398 | 25,900 |
| A41 | 16 | 9,700 |
| A42 | 42 | 61,900 |
| A42a | 24 | 78,700 |
| A42b | 285 | 118,000 |
| A43 | 79 | 101,000 |
| A44 | 34 | 51,200 |
| A45 | 410 | 79,300 |
| A46 | 301 | >150,000 |
| A47 | 518 | >150,000 |
| A48 | 29 | 44,600 |
| A49 | 35 | 123,000 |
| A49a | 6 | 77,900 |
| A49b | 400 | >150,000 |
| A50a | 4 | 24,700 |
| A50b | 332 | 13,600 |
| A51a | 3 | 14,000 |
| A51b | 265 | 22,300 |
| A52a | 32 | >150,000 |
| A52b | 1,030 | >150,000 |
| A53a | 24 | 60,300 |
| A53b | 662 | >150,000 |
| A54 | 37 | >150,000 |
| A55 | 31 | 68,700 |
| A56a | 810 | 108,000 |
| A56b | 23 | 50,900 |
| A57 | 3,360 | >150,000 |
| A58 | 40 | >10,000 |
| A58a | 20 | 13,300 |
| A58b | 1,660 | 90,800 |
| A59a | 238 | 40,300 |
| A59b | 2.3 | 14,200 |
| A60a | 3.3 | 72,500 |
| A60b | 206 | >150,000 |
| A61a | 2 | 10,100 |
| A61b | 420 | 37,900 |
| A62 | 279 | >150,000 |
| A63a | 2 | 10,300 |
| A63b | 22 | 7,400 |
| A64a | 0.88 | 5,280 |
| A64b | 14 | 4,750 |
| A65 | 2.3 | 24,200 |
| A66a | 217 | 15,800 |
| A66b | 1 | 6,860 |
| A67 | 10 | 118,000 |
| A68 | 7 | 43,800 |
| A68a | 2 | 20,300 |
| A68b | 199 | 72,800 |
| A69 | 6 | 3,070 |
| A69a | 2 | 1,190 |
| A69b | 147 | 6,090 |
| A70 | 8 | 9,690 |
| A71 | 48 | 124,000 |
| A72 | 64 | >150,000 |
| A73 | 639 | >150,000 |
| A74 | 45 | >150,000 |
| A74a | 46 | >150,000 |
| A74b | 4,870 | >150,000 |
| A75 | 253 | >150,000 |
| A76 | 419 | >150,000 |
| A77 | 6 | 19,700 |
| A78 | 7 | 102,000 |
| A79 | 16 | 16,400 |
| A79a | 38 | 7,280 |
| A79b | 529 | 14,800 |
| A80 | 31 | 95,000 |
| A80a | 7 | 65,600 |
| A80b | 664 | >150000 |
| A81 | 7 | >150,000 |
| A82 | 201 | 9,520 |
| A83 | 40 | >150,000 |
| A84 | 232 | >150,000 |
| A85 | 25 | 103,000 |
| A85a | 1,250 | 148,247 |
| A85b | 14 | 88,617 |
| A86 | 67 | >150,000 |
| A87 | 2 | 21,900 |
| A88 | 4 | 111,000 |
| A89 | 334 | >150,000 |
| A90 | 213 | >150,000 |
| A91 | 20 | >150,000 |
| A92 | 123 | >150,000 |
| A93 | 414 | >150,000 |
| A94 | 14 | 23,200 |
| A94a | 6 | 16,600 |
| A94b | 532 | >150,000 |
| A95 | 42 | 50,500 |
| A95a | 11 | 24,700 |
| A95b | 555 | 108,000 |
| A96 | 63 | 49,600 |
| A97 | 11 | 51,100 |
| A98 | 5 | 11,600 |
| A98a | 5 | 14,300 |
| A98b | 262 | 19,700 |
| A99 | 128 | 55,600 |
| A99a | 71 | 34,500 |
| A99b | 536 | 46,500 |
| A100 | 4,110 | >150,000 |
| A101 | 4,230 | >150,000 |
| A102 | 37 | >150,000 |
| A103a | 524 | >150,000 |
| A103b | 52 | >150,000 |
| A104 | 839 | >150,000 |
| A105 | 767 | >150,000 |
| A106 | >10,000 | >150,000 |
| A107 | >10,000 | >150,000 |
| A108 | 32 | 95,100 |
| A108a | 7 | 65,200 |
| A108b | 822 | 95,800 |
| A109 | 161 | 52,400 |
| A110 | 7 | >150,000 |
| A111 | 771 | >150,000 |
| A112 | 35 | >150,000 |
| A113 | 18 | >150,000 |
| A114 | 246 | 135,000 |
| A115 | 19 | >150,000 |
| A116 | 350 | >150,000 |
| A116a | 7,760 | >150,000 |
| A116b | 228 | >150,000 |
| A117a | 3,300 | >150,000 |
| A117b | 75 | 145,000 |
| A118a | 4,820 | >150,000 |
| A118b | 103 | 65,600 |
| A119 | 172 | >150,000 |
| A120 | 1,280 | >150,000 |
| A121 | 212 | >150,000 |
| A122 | 834 | >150,000 |
| A123 | 49 | 128,000 |
| A124 | 48 | 111,000 |
| A125 | 42 | >150,000 |
| A126 | 24 | 124,000 |
| A127 | 428 | >150,000 |
| A128 | 203 | 94,000 |
| A129 | 4 | >150,000 |
| A130 | 3,900 | >150,000 |
| A131 | 5,850 | >150,000 |
| A132 | 1,580 | >150,000 |
| A133 | 111 | >150,000 |
| A134 | 22 | 113,000 |
| A135 | 14 | >150,000 |
| A136 | 2 | >150,000 |
| A137 | 64 | 48,700 |
| A138 | 949 | >150,000 |
| A139 | 2,190 | 120,000 |
| A140 | 594 | >150,000 |
| A141 | 1,290 | >150,000 |
| A142 | 6,300 | >150,000 |
| A143 | >10,000 | >150,000 |
| A144 | 60 | 74,100 |
| A145 | 480 | 5,620 |
| A146a | 4,190 | 9,150 |
| A146b | 290 | 610 |
| A147 | 1,100 | 2,580 |
| A148a | 1,100 | 3,630 |
| A148b | 176 | 666 |
| A149a | 37 | 1,620 |
| A149b | 15 | 308 |
| A150 | 789 | 1,180 |
| A151a | 187 | 113 |
| A151b | 1,950 | 529 |
| A152 | 707 | 2,690 |
| A153a | 1,420 | 1,490 |
| A153b | 21 | 1,270 |
| A154 | 178 | 2,430 |
| A155 | 1,210 | 101,000 |
| A156a | 496 | 68,600 |
| A156b | 6 | 14,100 |
| A157a | 28 | 11,600 |
| A157b | 346 | 55,700 |
| A158 | 33 | 122,000 |
| A158a | 8 | 61,900 |
| A158b | 407 | >150,000 |
| A159 | 11 | 31,500 |
| A159a | 25 | 27,400 |
| A159b | 691 | 63,400 |
| A160 | 331 | >150,000 |
| A161 | 81 | >150,000 |
| A16la | 24 | 71,800 |
| A161b | 2,630 | >150,000 |
| A162 | 33 | 60,500 |
| A163 | 48 | >150,000 |
| A163a | 13 | >150,000 |
| A163b | 2,070 | >150,000 |
| A164 | 31 | 125,000 |
| A165 | 69 | >150,000 |
| A166 | 14 | 26,100 |
| A167 | 90 | 19,000 |
| A168 | 26 | 64,300 |
| A169 | 31 | 28,500 |
| A169a | 10 | 31,700 |
| A169b | 455 | 78,200 |
| A170 | 1,180 | >150,000 |
| A171 | 107 | 65,800 |
| A172a | 170 | 132,000 |
| A172b | 8 | 116,000 |
| A173 | 44 | 143,000 |
| A173a | 12 | 51,000 |
| A173b | 412 | 83,300 |
| A174a | 1,030 | 51,200 |
| A174b | 69 | 30,600 |
| A175 | 5 | 22,200 |
| A175a | 291 | 37,200 |
| A175b | 4 | 21,400 |
| A176a | 40 | 13,100 |
| A176b | 7 | 7,190 |
| A177 | 168 | 72,600 |
| A178 | 15 | 92,400 |
| A179 | 6 | 69,000 |
| A180 | 4,440 | >150,000 |
| A181 | 17 | >150,000 |
| A182 | 499 | >150,000 |
| A183 | 4 | >150,000 |
| A184 | 6 | 88,500 |
| A185a | 806 | 121,000 |
| A185b | 20 | 82,500 |
| A186 | 156 | 143,000 |
| A187 | 407 | 115,000 |
| A188 | 74 | >150,000 |
| A189 | 34 | >150,000 |
| A190 | 13 | 106,000 |
| A190a | 3 | >150,000 |
| A190b | 41 | 38,200 |
| A191 | 78 | 40,900 |
| A192 | 119 | >150,000 |
| A193 | >10,000 | >150,000 |
| A194 | 7 | 4,080 |
| A194a | 7 | 3,950 |
| A194b | 873 | 7,020 |
| A195 | 25 | >150,000 |
| A196 | 749 | >10,000 |

### Cell Culture and DGKα/Knockout Jurkat Cell Line Construction

Jurkat cells and human PBMC were maintained in RPMI 1640 medium (Gibco) supplemented with 10% fetal bovine serum (FBS, Thermo Scientific), 100 units/mL penicillin and 0.1 mg/mL streptomycin (Gibco) in a humidified 37° C. environment with 500 COz. HepG2-OS8 cells, which express the single chain variable fragment (scFv) of an anti-human CD₃ mAb OKT3 fused to the C-terminal domain (113-220) of mouse CD₈a which includes hinge, transmembrane and cytoplasmic domains, were maintained in MEM medium (Gibco) supplemented with 10% fetal bovine serum (FBS, Thermo Scientific), 100 units/mL penicillin and 0.1 mg/mL streptomycin (Gibco) in a humidified 37° C. environment with 5% CO₂.

Jurkat cells were infected with the lentivirus expressing spCas9 and sgRNA targeting human DGKα or DGKζ. Cell clones that stably knockout with DGKα/ζ were established and maintained in the RPMI 1640 complete medium. Knockout efficiency of eSPCas9-Lenticrispr DGKα or DGK sgRNA in single cell clone was determined using genomic sequencing and immunoblotting method. Selected compounds did not induce DGKα or DGK independent IL-2 production in DGKα/ζ KO jurkat cells.

### Non-Stimulated Phosphorylated ERK Detection Assay

Cellular non-stimulated phospho-ERK were measured using a AlphaLISA-based method (Beaudet, Lucille, et al. Nature Methods. 2008, 5.12: an8-an9). Jurkat cells were subcultured in T75 flasks. The next day, growth medium was replaced to serum free RPMI 1640 for 4 hours or overnight. The cells were then seeded into 96-well plates and treated with compounds. After 2 h compound treatment, lysis buffer (PerkinElmer) was added to each well. Plates were then incubated at room temperature with shaking for 30 minutes. A total of 10 μL of cell lysate from each well of a 96-well plate was transferred to a 384-well white assay plate. Phosphor-ERK was quantitated using the AlphaLISA kit (Cat #ALSU-PERK-A10K) as described by the manufacturer manual (PerkinElmer). AlphaLISA signals were measured using a PHERAstar FSX reader (BMG Labtech). Selected compounds did not elevate ERK phosphorylation in Jurkat cells without TCR activation.

### IL-2 Production Assay in Human PBMC

Frozen human PBMC were thawed in RPMI 1640 medium and incubated at 37° C. overnight. OS8 overexpressing HepG2 cells were seeded into 384-well plates overnight. The next day, PBMC were added into the 384-well plates and then treated with compounds. PBMC and HepG2-OS8 cells were co-cultured for 48 hours at 37° C. Culture supernatant was collected for subsequent measurement of IL-2 concentration by a TR-FRET-based method (Degorce, Frangois, et al. Current chemical genomics. 2009, 3: 22) as described by the manufacturer manual (Cisbio). FRET signals were measured using a PHERAstar FSX reader (BMG Labtech). Selected compounds showed good potency in Human PBMC assay.

### Explorative Acute Toxicity Study in BALB/c Mice

The test compound was dissolved in vehicle formulation (DMA: 30% solutol HS-15 (w/v): saline=20:20:60) and injected through tail vein into BALB/c mice at the doses of 2 and/or 10 mg/kg. Continuous clinical observation within 2 hours post injection was performed. Selected compounds were well tolerant at the doses of 2 and/or 10 mg/kg.

It is to be understood that, if any prior art publication is referred to herein, such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

The disclosures of all publications, patents, patent applications and published patent applications referred to herein by an identifying citation are hereby incorporated herein by reference in their entirety.

What is claimed is:

1. A compound of formula (I), 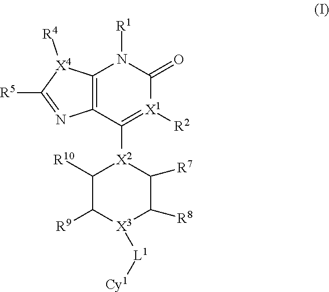
or a pharmaceutically acceptable salt, a tautomer, a stereoisomer, an enantiomer, an isotopologue, or a prodrug thereof,
wherein
X¹ is C or N,
each of X² and X³ is independently selected from —N— or —CH—;
X₄ is N, O or S;
R¹ is hydrogen, deuterium, halogen, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl;
R² is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl, provided that R² is absent when X¹ is N;
R⁴ is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl, provided that R⁴ is absent when X⁴ is O or S;
R⁵ is hydrogen, halogen, cyano, substituted or unsubstituted alkyl, substituted or unsubstituted alkoxyl, substituted or unsubstituted alkenyl, substituted or unsubstituted alkynyl, substituted or unsubstituted cycloalkyl, substituted or unsubstituted aryl, substituted or unsubstituted heterocyclyl, or substituted or unsubstituted heteroaryl, R^{5a}—C(O)—, R^{5a}—C(O)O—, R^{5a}—O—C(O)—, R^{5a}—C(O)NR^{5b}—, R^{5a}—NR^{5b}—C(O)—, or R^{5a}—SO₂—, wherein R^{5a} and R^{5b} are each independently hydrogen, alkyl, or cycloalkyl;
each of R⁷, R⁹, R⁸, and R¹⁰ is independently hydrogen, cyano, substituted or unsubstituted alkyl, substituted or unsubstituted alkoxyl, substituted or unsubstituted cycloalkyl, —C(O)R^{7a}, or -alkyl-C(O)R^{7a}, and wherein R^{7a} is hydrogen, alkyl, or alkoxy, provided that at least one of R⁷ and R⁹ is not hydrogen;
or R⁷ and R⁹ are each hydrogen and R⁸ and R¹⁰ together form a bridge containing at least one —CH₂-moiety in addition to the two bridgehead atoms; or R⁸ and R¹⁰ are each hydrogen and R⁷ and R⁹ together form a bridge containing at least one —CH₂— moiety in addition to the two bridgehead atoms;
L¹ is a direct bond, —O—, —N(R^{L})—, substituted or unsubstituted alkyl, -alkylene or —C(O)—, wherein R^{L} is hydrogen or alkyl;
Cy¹ is unsubstituted or substituted cycloalkyl, unsubstituted or substituted aryl, unsubstituted or substituted heterocyclyl, or unsubstituted or substituted heteroaryl, wherein Cy¹ is optionally substituted with 1 to 5 substituents R^{3a},
wherein R^{3a} is selected from deuterium, alkoxy, alkyl, halogen, oxo, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, cycloalkyl or heterocyclyl;
optionally wherein two R^{3a} connect to the same carbon and together form a spirocyclic ring;
optionally wherein two R^{3a} form a fused ring with Cy¹, wherein R^{3a} is unsubstituted or substituted with cyano, alkoxy, alkyl, halogen, or hydroxy; and
wherein R^{3b} and R^{3c} are each independently hydrogen or alkyl.

2. The compound according to claim 1, which is a compound of any one of formulas (IA1), (IB1) or (IC1): 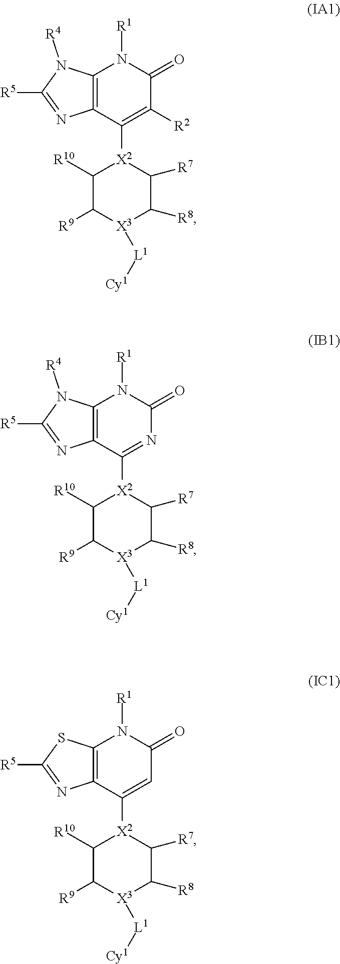
wherein the variables are defined as herein.

3. The compound according to claim 1, which is a compound of any one of formula (IA2), (IB2) or (IC2): 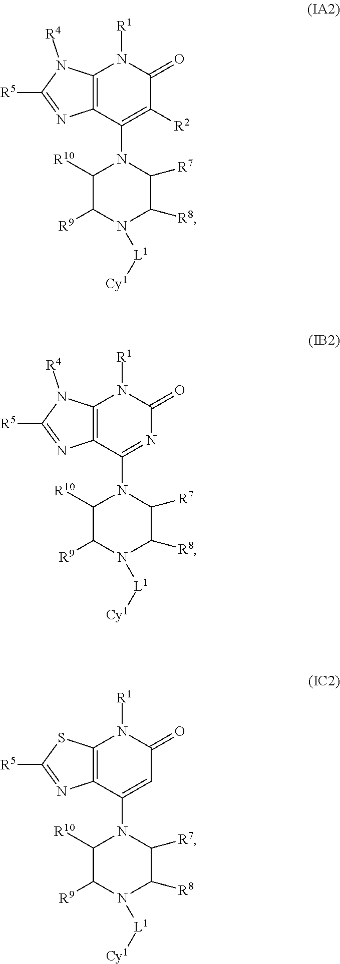
wherein the variables are defined as herein.

4. The compound according to claim 1, which is a compound of any one of formula (IA3), (IB3) or (IC3): 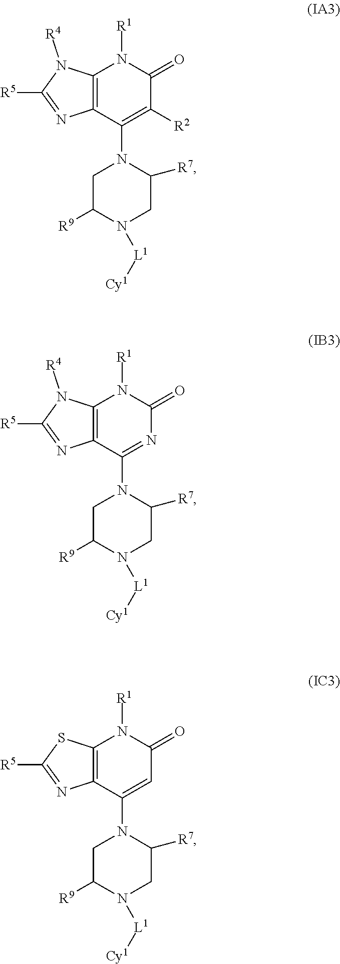
wherein the variables are defined as herein.

5. The compound according to claim 1, which is a compound of any one of formula (IA4), (IB4) or (IC4): 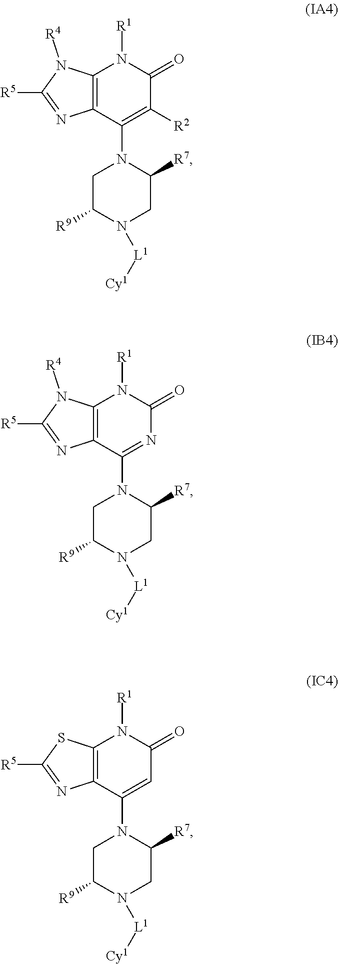
wherein the variables are defined as herein.

6. The compound according to claim 1, which is a compound of formula (II): 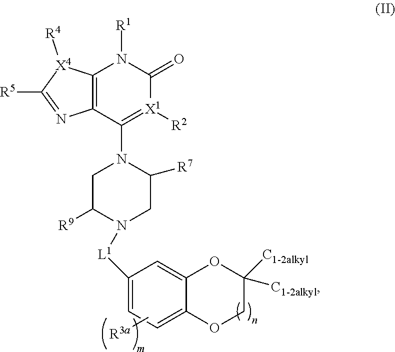
or a pharmaceutically acceptable salt, a tautomer, a stereoisomer, an enantiomer, an isotopologue, or a prodrug thereof,
wherein
X¹ is C or N,
X⁴ is N, O or S;
R¹ is hydrogen, deuterium, halogen, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl;
R² is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl, provided that R² is absent when X¹ is N;
R⁴ is hydrogen, halogen, cyano, substituted or unsubstituted alkyl, substituted or unsubstituted alkoxyl, substituted or unsubstituted cycloalkyl, substituted or unsubstituted aryl, substituted or unsubstituted heterocyclyl, or substituted or unsubstituted heteroaryl, provided that R⁴ is absent when X⁴ is O or S;
R is hydrogen, halogen, cyano, substituted or unsubstituted alkyl, substituted or unsubstituted alkoxyl, substituted or unsubstituted alkenyl, substituted or unsubstituted alkynyl, substituted or unsubstituted cycloalkyl, substituted or unsubstituted aryl, substituted or unsubstituted heterocyclyl, or substituted or unsubstituted heteroaryl, R^{5a}—C(O)—, R^{5a}—C(O)O—, R^{5a}—O—C(O)—, R^{5a}—C(O)NR^{5b}—, R^{5a}—NR^{5b}—C(O)—, or R^{5a}—SO₂—, wherein R^{5a} and R^{5b} are each independently hydrogen, alkyl, or cycloalkyl;
each of R⁷, and R⁹ is independently hydrogen, cyano, substituted or unsubstituted alkyl, substituted or unsubstituted alkoxyl, substituted or unsubstituted cycloalkyl, —C(O)R^{7a}, or -alkyl-C(O)R^{7a}, and wherein R^{7a} is hydrogen, alkyl, or alkoxy, provided that at least one of R⁷ and R⁹ is not hydrogen;
or R⁷ and R⁹ together form a bridge containing at least one —CH₂— moiety in addition to the two bridgehead atoms;
L¹ is a direct bond, —O—, —N(R^{L})—, substituted or unsubstituted alkyl, -alkylene or —C(O)—, wherein R^{L} is hydrogen or alkyl;
m is 0, 1, 2 or 3,
n is 0 or 1
wherein R^{3a} is selected from deuterium, alkoxy, alkyl, halogen, oxo, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, cycloalkyl or heterocyclyl;
optionally wherein two R^{3a} connect to the same carbon and together form a spirocyclic ring;
optionally wherein two R^{3a} form a fused ring with Cy¹, wherein R^{3a} is unsubstituted or substituted with cyano, alkoxy, alkyl, halogen, or hydroxy; and
wherein R^{3b} and R^{3c} are each independently hydrogen or alkyl.

7. The compound according to any one of claims 1 to 6, wherein R¹ is hydrogen, or substituted or unsubstituted alkyl.

8. The compound according to any one of claims 1 to 7, wherein R¹ is hydrogen, or C₁₋₄alkyl optionally substituted with deuterium, halogen, hydroxy, alkoxy or cycloalkyl; preferably R¹ is hydrogen, or C₁₋₄alkyl optionally substituted with deuterium, halogen, hydroxy, alkoxy or cycloalkyl; more preferably R¹ is hydrogen, or C₁₋₃alkyl optionally substituted with deuterium, or halogen; even more preferably R¹ is hydrogen, methyl, methyl-d3, ethyl, isopropyl, 2-hydroxyethyl, 2-methoxyethyl, 2,2,2-trifluoroethyl, 2,2-difluoroethyl, or cyclopropylmethyl.

9. The compound according to claim 8, wherein R² is hydrogen, halogen, C₁₋₄alkyl, C₁₋₄ alkoxyl or cyano; preferably R² is hydrogen, F, Br, Cl or CN.

10. The compound according to any one of claims 1 to 9, wherein R⁴ is hydrogen, halogen or alkyl, wherein the alkyl is optionally substituted with deuterium, halogen or —OR^{4a}, wherein R^{4a} is hydrogen, alkyl, alkenyl, alkynyl, cycloalkyl or heterocycloalkyl, wherein alkyl, alkenyl, alkynyl, cycloalkyl or heterocycloalkyl is optionally substituted with —C₁₋₆alkyl, —C₁₋₆alkoxy or —C₃₋₈cycloalkyl; preferably R⁴ is hydrogen, or C₁₋₃alkyl optionally substituted with deuterium, or halogen; more preferably R⁴ is hydrogen, fluoro, chloro, bromo, methyl, methyl-d3, trifluoromethyl, ethyl, 2,2-difluoroethyl, 2,2,2-trifluoroethyl, propyl, cyclopropyl.

11. The compound according to any one of claims 1 to 10, wherein R⁵ is hydrogen, alkyl, alkenyl or alkynyl, wherein said alkyl is unsubstituted or substituted with cyano; preferably R⁵ is C₁₋₄alkyl, C₂₋₄ alkenyl or C₂₋₄alkynyl, wherein said alkyl is substituted with cyano; more preferably R⁵ is C₁₋₄alkyl, wherein said alkyl is substituted with cyano.

12. The compound according to any one of claims 1 to 11, wherein each of R⁷ and R⁹ is independently hydrogen, alkyl, or —C(O)R^{7a}, wherein said alkyl is unsubstituted or substituted with halogen, cyano, hydroxy, or alkoxy, and wherein R^{7a} is hydrogen, alkyl, or alkoxy; preferably each of R⁷ and R⁹ is independently C₁₋₂alkyl.

13. The compound according to claim 12, wherein R⁷ and R⁹ are each independently hydrogen, methyl, ethyl, methoxymethyl, 1-hydroxyethyl, 2-methoxyethyl, cyanomethyl, hydroxyethyl, hydroxymethyl, methoxycarbonyl, difluoromethyl, provided that at least one of R⁷ and R⁹ is not hydrogen.

14. The compound according to any one of claims 1 to 13, wherein R⁸ and R¹⁰ are each hydrogen; R⁷ is methyl; and R⁹ is methyl; R⁸ and R¹⁰ are each hydrogen; R⁷ is ethyl; and R⁹ is ethyl; R⁸ and R¹⁰ are each hydrogen; R⁷ is methyl; and R⁹ is ethyl; or R⁸ and R¹⁰ are each hydrogen; R⁷ is ethyl, and R⁹ is methyl; R⁸ and R¹⁰ are each hydrogen; R⁷ is methoxymethyl, and R⁹ is methyl, ethyl or methoxymethyl.

15. The compound according to any one of claims 1 to 14, wherein L¹ is a direct bond, —O—, —N(R^{L})—, -alkylene- or —C(O)—, wherein R^{L} is hydrogen or alkyl and wherein said -alkylene- is unsubstituted or substituted with deuterium, halogen, alkoxy, alkynyl or heterocyclyl; preferably L¹ is a direct bond, —O—, —N(R^{L})—, -alkylene- or —C(O)—, wherein R^{L} is hydrogen or alkyl; preferably L¹ is C₁₋₄alkylene, preferably C₁₋₂alkylene; more preferably L¹ is a direct bond, —CH₂—, —CH(CH₃)—, —CH(CD₃)—, —CH(CH₂CH₃)—, —CH(CHF₂)—, —CH(prop-1-ynyl)-, —N(H)—, —N(CH₃)—, —O—, —CH(C(O)—NHCH₂CH₂OCH₃)—or —C(CH₃)₂—.

16. The compound according to any one of claims 1 to 5 and 7 to 15, wherein Cy¹ is aryl, heterocyclyl, heterocyclyl, heteroaryl, or cycloalkyl, each of which is unsubstituted or substituted with one, two, three, or four substituents R^{3a}, wherein R^{3a} is selected from deuterium, alkoxy, alkyl, halogen, R^{3b}—SO₂—, cycloalkyl, cyano, R^{3b}-C(O)—N(R^{3c})—, N(R^{3b}R^{3c})—C(O)—, N(R^{3b}R^{3c}), R^{3b}O—C(O)—, or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl.

17. The compound according to claim 16, wherein Cy¹ is unsubstituted or substituted cycloalkyl, unsubstituted or substituted aryl, unsubstituted or substituted heterocyclyl, or unsubstituted or substituted heteroaryl; preferably Cy¹ is optionally substituted with one, two or three substituents R^{3a}, wherein R^{3a} is selected from deuterium, fluoro, bromo, chloro, methyl, difluoromethyl, trifluoromethoxy, methoxy, methoxymethyl, trifluoromethyl, methylsulfonyl, difluoro, ethoxy, isopropyl, isopropoxy, difluoromethoxy, cyclopropyl, 2,2-difluorocyclopropyl, 1-fluorocyclopropyl, 2-fluorocyclopropyl, cyclobutyl, 1-hydroxyethyl, 2-hydroxyethyl, ethyl, 1,1-difluoroethyl, cyano, dimethoxy, dichloro, acetamido, 1-methoxyethyl, cyanomethyl, carbamoyl, methoxycarbonyl, dimethylcarbamoyl, (difluoromethoxy)methyl, amino, 1-(difluoromethoxy)ethyl, azetidin-1-yl, 2-methoxypropan-2-yl, 1-methoxycyclopropyl, oxetan-3-yl, 1-methylazetidin-3-yl, 1-hydroxyazetidin-3-yl, 2-cyanoethyl, 2-cyanopropan-2-yl, hydroxymethyl, 3-hydroxypropyl, 2-hydroxypropan-2-yl, or 2-fluoropropan-2-yl.

18. The compound according to claim 16, wherein Cy¹ is aryl, which is unsubstituted or substituted with one, two or three substituents R^{3a}, wherein R^{3a} is selected from alkoxy, halogen-substituted alkoxy, alkoxyalkyl-, alkyl, alkenyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, heterocyclylalkyl- or heterocyclyl, said cycloalkyl or heterocyclyl is unsubstituted or substituted with alkoxy, alkyl, halogen, or hydroxy, wherein R^{3b} and R^{3c} are each independently hydrogen or alkyl.

19. The compound according to claim 18, wherein Cy¹ is phenyl, which is substituted with one or two R^{3a}, wherein R^{3a} is fluoro, chloro, bromo; methyl, difluoromethyl, trifluoromethyl, 1,1-difluoroethyl, methoxymethyl, 1-methoxyethyl, ethyl, propyl, isopropyl, tert-butyl, 2-hydroxypropan-2-yl, 1-hydroxy-2-methylpropan-2-yl, 2-cyanopropan-2-yl, morpholinomethyl; prop-1-en-2-yl; cyclopropyl, 1-methylcyclopropyl; methoxy, difluoromethoxy, trifluoromethoxy, 1,1-difluoroethoxy, isopropoxy; oxetan-3-yl, morpholino, 2-oxa-6-azaspiro[3.3]heptan-6-yl; or methylcarbamoyl.

20. The compound according to claim 16, wherein Cy¹ is a monocyclic 5- to 9-membered heterocyclyl or a bicyclic 7- to 10-membered heterocyclyl which is unsubstituted or substituted with one, two, three or four R^{3a} (provided that the valency theory has been met), wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, oxo or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably, R^{3a} is selected from alkoxy, halogen-substituted alkoxy, alkoxyalkyl-, alkyl, halogen-substituted alkyl, halogen, R^{3b}—SO₂—, cycloalkyl, hydroxyalkyl-, cyano, R^{3b}—C(O)—N(R^{3c})—, cyano-substituted alkyl, N(R^{3b}R^{3c})—C(O)—, R^{3b}—O—C(O)—, or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; preferably, said monocyclic 5- to 9-membered heterocyclyl is tetrahydrofuranyl, tetrahydropyranyl, 1,4-dioxanyl, piperidinyl, piperazinyl, or dihydropyridinyl, each of which is unsubstituted or substituted with one, two or three R^{3a} as disclosed herein; preferably Cy¹ is tetrahydrofuran-2-yl, tetrahydrofuran-3-yl, tetrahydro-2H-pyran-2-yl, tetrahydro-2H-pyran-3-yl, tetrahydro-2H-pyran-4-yl, 1,4-dioxan-2-yl, 1,4-dioxan-3-yl, piperidin-1-yl, piperidin-2-yl, piperidin-3-yl, piperidin-4-yl, piperazin-1-yl, piperazin-2-yl, piperazin-3-yl, piperazin-4-yl, 1,2-dihydropyridin-3-yl, 1,2-dihydropyridin-4-yl, 1,2-dihydropyridin-5-yl, or 1,2-dihydropyridin-6-yl.

21. The compound according to claim 16, wherein Cy¹ is dihydrobenzodioxinyl, benzodioxolyl, chromanyl, or chromenyl, which is unsubstituted or substituted with one, two, three or three R^{3a}, wherein R^{3a} is selected from alkoxy, alkyl, halogen, and oxo; preferably, Cy¹ is 2,3-dihydrobenzo[b][1,4]dioxin-5-yl, 2,3-dihydrobenzo[b][1,4]dioxin-6-yl, benzo[d][1,3]dioxol-4-yl, benzo[d][1,3]dioxol-5-yl, chroman-2-yl, chroman-3-yl, chroman-4-yl, chroman-5-yl, chroman-6-yl, chroman-7-yl, chromen-2-yl, chromen-3-yl, chromen-4-yl, chromen-5-yl, chromen-6-yl, chromen-7-yl, each of which is unsubstituted or substituted with one, two, three or three R^{3a}, wherein R^{3a} is selected from alkoxy, alkyl, halogen, and oxo; preferably R^{3a} is methyl, ethyl, propyl, isopropyl, fluoro, chloro, bromo or oxo.

22. The compound according to claim 16, wherein Cy¹ is a monocyclic 5- to 9-membered heteroaryl or a bicyclic 7- to 10-membered heteroaryl which is unsubstituted or substituted with one, two or three R^{3a}, wherein R^{3a} is selected from deuterium, alkoxy, alkyl, halogen, R^{3b}—SO₂—, cycloalkyl, cyano, R^{3b}—C(O)—N(R^{3c})—, N(R^{3b}R^{3c})—C(O)—, N(R^{3b}R^{3c}), R^{3b}—O—C(O)—, oxo, phenyl or heterocyclyl, wherein said alkyl moiety in the group alkyl or alkoxy is unsubstituted or substituted with halogen, alkoxy, hydroxy, or cyano; said cycloalkyl or heterocyclyl is unsubstituted or substituted with alkoxy, alkyl, halogen, or hydroxy, wherein R^{3b} and R^{3c} are each independently hydrogen or alkyl; preferably, R^{3a} is selected from deuterium, alkoxy, halogen-substituted alkoxy, alkoxyalkyl-, alkyl, halogen-substituted alkyl, halogen, oxo, hydroxyalkyl-, cyano, cyano-substituted alkyl, N(R^{3b}R^{3c})—C(O)—, N(R^{3b}R^{3c})—, R^{3b}—O—C(O)—, oxo, phenyl or heterocyclyl, said cycloalkyl or heterocyclyl is unsubstituted or substituted with alkoxy, alkyl, halogen, or hydroxy, wherein R^{3b} and R^{3c} are each independently hydrogen or alkyl; more preferably R^{3a} is selected from deuterium, fluoro, bromo, chloro, methyl, difluoromethyl, trifluoromethyl, trifluoromethoxy, methoxy, methoxymethyl, methylsulfonyl, ethoxy, isopropoxy, isopropyl, tert-butyl, cyclopropyl, 1-hydroxyethyl, ethyl, 1,1-difluoroethyl, cyano, cyclopropyl-, acetamido, 1-methoxyethyl, cyanomethyl, carbamoyl, methoxycarbonyl, methylcarbamoyl, dimethylcarbamoyl, (difluoromethoxy)methyl, amino, isopropylamino, (difluoromethoxy)ethyl, azetidin-1-yl, 2-methoxypropan-2-yl, 1-methoxycyclopropyl, oxetan-3-yl, 1-methylazetidin-3-yl, 1-hydroxyazetidin-3-yl, phenyl or oxo.

23. The compound according to claim 16, wherein Cy¹ is pyrazolyl, imidazolyl, thiazolyl, pyridinyl, pyrimidinyl, pyrazinyl or pyridazinyl, each of which is unsubstituted or substituted with one, two or three R^{3a} as disclosed herein; preferably Cy¹ is 1H-pyrazol-1-yl, 1H-pyrazol-3-yl, 1H-pyrazol-4-yl, thiazol-4-yl, thiazol-5-yl, 1H-pyrazol-5-yl, 1H-imidazol-2-yl, 1H-imidazol-4-yl, 1H-imidazol-5-yl, pyridin-2-yl, pyridin-3-yl, pyridin-4-yl, pyrimidin-2-yl, pyrimidin-4-yl, pyrimidin-5-yl, 1H-indol-2-yl, 1H-indol-3-yl, 1H-indol-4-yl, 1H-indol-5-yl, 1H-indol-6-yl, 1H-indol-7-yl, pyrazin-2-yl, or pyridazin-4-yl, each of which is unsubstituted or substituted with one, two or three R^{3a} as disclosed herein; preferably R^{3a} is selected from deuterium, fluoro, bromo, chloro, methyl, difluoromethyl, trifluoromethyl, trifluoromethoxy, methoxy, ethoxy, isopropoxy, isopropyl, tert-butyl, carbamoyl, methylcarbamoyl, dimethylcarbamoyl, isopropylamino, azetidin-1-yl, phenyl or oxo.

24. The compound according to claim 16, wherein Cy¹ is indolyl, triazolopyridinyl, imidazopyridinyl, benzooxazolyl, benzo[d]thiazolyl, quinolinyl, isoquinolinyl, naphthyridinyl, dioxinopyridineyl, quinoxalinyl, benzo[d]imidazolyl, benzofuranyl, benzoxazinyl, imidazo[4,5-b]pyridinyl, thiazolo[5,4-b]pyridinyl, thiazolo[4,5-b]pyridinyl, thieno[2,3-b]pyridinyl, dihydrodioxinopyridinyl, dioxinopyridinyl, dihydro-2H-pyranopyridinyl, 2H-pyranopyridinyl, dihydrofuropyridinyl, furopyridinyl, benzo[d]thiazolyl, isoindolinyl or thieno[3,2-b]pyridinyl, each of which is unsubstituted or substituted with one, two or three R^{3a} as disclosed herein; preferably Cy¹ is quinoxalin-6-yl, quinoxalin-2-yl, isoquinolin-3-yl, isoquinolin-7-yl, quinolin-6-yl, 1H-benzo[d]imidazol-5-yl, benzofuran-6-yl, benzofuran-5-yl, benzo[e][1,3]oxazin-6-yl, 2,3-dihydro-[1,4]dioxino[2,3-b]pyridin-6-yl, 2,3-dihydro-[1,4]dioxino[2,3-b]pyridin-7-yl, 3,4-dihydro-2H-pyrano[3,2-b]pyridin-6-yl, 3,4-dihydro-2H-pyrano[3,2-b]pyridin-8-yl, 2,3-dihydrofuro[2,3-b]pyridin-6-yl, 2,3-dihydrofuro[3,2-b]pyridin-5-yl, benzo[d]thiazol-5-yl, benzo[d]thiazol-6-yl, isoindolin-5-yl, 1H-benzo[d]imidazol-2-yl, 1H-benzo[d]imidazol-4-yl, 1H-benzo[d]imidazol-6-yl, 1H-benzo[d]imidazol-7-yl, [1,2,4]triazolo[1,5-a]pyridin-2-yl, [1,2,4]triazolo[1,5-a]pyridin-5-yl, [1,2,4]triazolo[1,5-a]pyridin-6-yl, [1,2,4]triazolo[1,5-a]pyridin-7-yl, [1,2,4]triazolo[1,5-a]pyridin-8-yl, 3H-imidazo[4,5-b]pyridine-2-yl, 3H-imidazo[4,5-b]pyridine-5-yl, 3H-imidazo[4,5-b]pyridine-6-yl, 3H-imidazo[4,5-b]pyridine-7-yl, 1H-imidazo[4,5-b]pyridin-2-yl, 1H-imidazo[4,5-b]pyridin-5-yl, 1H-imidazo[4,5-b]pyridin-6-yl, 1H-imidazo[4,5-b]pyridin-7-yl, benzo[d]oxazol-2-yl, benzo[d]oxazol-4-yl, benzo[d]oxazol-5-yl, benzo[d]oxazol-6-yl, benzo[d]oxazol-7-yl, benzo[d]thiazol-2-yl, benzo[d]thiazol-4-yl, benzo[d]thiazol-7-yl, quinolin-2-yl, quinolin-3-yl, quinolin-4-yl, quinolin-5-yl, quinolin-7-yl, isoquinolin-1-yl, isoquinolin-4-yl, isoquinolin-5-yl, isoquinolin-6-yl, isoquinolin-8-yl, quinoxalin-3-yl, quinoxalin-4-yl, quinoxalin-5-yl, quinoxalin-7-yl, quinoxalin-8-yl, 1,8-naphthyridin-2-yl, 1,8-naphthyridin-3-yl, 1,8-naphthyridin-4-yl, 2,3-dihydro-[1,4]dioxino[2,3-b]pyridine-6-yl, 2,3-dihydro-[1,4]dioxino[2,3-b]pyridine-7-yl, 2,3-dihydro-[1,4]dioxino[2,3-b]pyridine-8-yl, quinoxalin-6-yl-2,3-d2, 1H-indol-2-yl, 1-methyl-1H-benzo[d]imidazol-6-yl, 3H-imidazo[4,5-b]pyridin-2-yl, 4,5,6,7-tetrahydro-1H-benzo[d]imidazol-2-yl, thiazolo[5,4-b]pyridin-2-yl, thiazolo[5,4-b]pyridin-5-yl, thiazolo[5,4-b]pyridin-6-yl, thiazolo[5,4-b]pyridin-7-yl, thiazolo[4,5-b]pyridin-2-yl, thiazolo[4,5-b]pyridin-5-yl, thiazolo[4,5-b]pyridin-6-yl, thiazolo[4,5-b]pyridin-7-yl, thieno[2,3-b]pyridin-2-yl, thieno[2,3-b]pyridin-3-yl, thieno[2,3-b]pyridin-4-yl, thieno[2,3-b]pyridin-5-yl, thieno[2,3-b]pyridin-6-yl, thieno[3,2-b]pyridin-2-yl, thieno[3,2-b]pyridin-3-yl, thieno[3,2-b]pyridin-5-yl, thieno[3,2-b]pyridin-6-yl, thieno[3,2-b]pyridin-7-yl, each of which is unsubstituted or substituted with one, two or three R^{3a} as disclosed herein.

25. The compound according to claim 16, wherein Cy¹ is quinoxalin-6-yl, quinoxalin-2-yl, benzo[d]thiazol-5-yl, benzo[d]thiazol-6-yl, isoindolin-4-yl, indolin-5-yl, 1H-benzo[d]imidazol-5-yl, benzofuran-6-yl, benzofuran-5-yl, or benzo[e][1,3]oxazin-6-yl, isoquinolin-3-yl, isoquinolin-7-yl, quinolin-6-yl, 2,3-dihydro-[1,4]dioxino[2,3-b]pyridin-6-yl, 2,3-dihydro-[1,4]dioxino[2,3-b]pyridin-7-yl, 3-dihydro-[1,4]dioxino[2,3-c]pyridin-7-yl, 3,4-dihydro-2H-pyrano[3,2-b]pyridin-6-yl, 3,4-dihydro-2H-pyrano[3,2-b]pyridin-8-yl, 2,3-dihydrofuro[2,3-b]pyridin-6-yl, 2,3-dihydrofuro[3,2-b]pyridin-5-yl, each of which is unsubstituted or substituted with one, two or three R^{3a}, wherein R^{3a} is deuterium, methyl, ethyl, isopropyl, cyanomethyl, 2-cyanoethyl, 2-cyanopropan-2-yl, hydroxymethyl, 2-hydroxyethyl, 1-hydroxyethyl, 3-hydroxypropyl, 2-hydroxypropan-2-yl, difluoromethyl, trifluoromethyl, 2,2,2-trifluoroethyl, 2-fluoropropan-2-yl, methoxy, isopropoxy, difluoromethoxy, fluoro, chloro, bromo, oxo, cyano, amino, cyclopropyl, 2,2-difluorocyclopropyl, 1-fluorocyclopropyl, 2-fluorocyclopropyl, or cyclobutyl; preferably deuterium, methyl, ethyl, trifluoromethyl, methoxy, isopropoxy, difluoromethoxy, fluoro, chloro, cyano, amino, or cyclopropyl.

26. The compound according to claim 16, wherein Cy¹ is
4-fluoro-2-methoxyphenyl, p-tolyl, 4-fluoro-2-(trifluoromethyl)phenyl, 2-(difluoromethoxy)-4-fluorophenyl, 4-fluoro-2-(trifluoromethoxy)phenyl, 4-fluoro-2-(methoxymethyl)phenyl, 4-fluoro-2-(1-methoxyethyl)phenyl, 1-(difluoromethoxy-4-fluorophenyl, 2-(difluoromethyl)-4-fluorophenyl, 2-(1,1-difluoroethoxy)-4-fluorophenyl, 3-(trifluoromethyl)phenyl, 3-isopropoxyphenyl, 3-(trifluoromethoxy)phenyl, 3-isopropylphenyl, 3-(tert-butyl)phenyl, 3-(2-hydroxypropan-2-yl)phenyl, 3-(difluoromethyl)-4-fluorophenyl, 4-fluoro-3-(trifluoromethyl)phenyl, 2-fluoro-5-(trifluoromethyl)phenyl, 4-methyl-3-(trifluoromethyl)phenyl, 4-methyl-2-(trifluoromethyl)phenyl, 3-cyclopropylphenyl, 3-(1-methylcyclopropyl)phenyl, 3-(oxetan-3-yl)phenyl, 4-fluoro-3-(methylcarbamoyl)phenyl, 3-(1-hydroxy-2-methylpropan-2-yl)phenyl, 3-(2-cyanopropan-2-yl)phenyl, 4-fluoro-3-(prop-1-en-2-yl)phenyl, 4-fluoro-3-(1-methylcyclopropyl)phenyl, 3-methyl-4-(trifluoromethyl)phenyl, 4-(morpholinomethyl)phenyl, 3-(2-oxa-6-azaspiro[3.3]heptan-6-yl)phenyl, 3-morpholinophenyl, 3-(morpholinomethyl)phenyl, 4-morpholinophenyl, 4-(trifluoromethyl)phenyl, 4-fluorophenyl, 3-methyl-2-(trifluoromethyl)phenyl, 3-(difluoromethyl)-5-fluorophenyl, 3-(1,1-difluoroethyl)-4-methoxyphenyl, m-tolyl, 3-methoxyphenyl, or 3-(difluoromethyl)phenyl; or
3,3-dimethyl-2,3-dihydro-1H-inden-5-yl; or
4,4-difluorochroman-6-yl, 2,2-dimethyl-4-oxochroman-7-yl, 3-methyl-4-oxo-4H-chromen-6-yl, 2,2-dimethylchroman-7-yl, or 2-methyl-4-oxo-4H-chromen-7-yl; or
2,3-dihydrobenzo[b][1,4]dioxin-6-yl, 3,3-dimethyl-2,3-dihydrobenzo[b][1,4]dioxin-6-yl, 8-fluoro-3,3-dimethyl-2,3-dihydrobenzo[b][1,4]dioxin-6-yl, 7-fluoro-3,3-dimethyl-2,3-dihydrobenzo[b][1,4]dioxin-6-yl, 5-fluoro-3,3-dimethyl-2,3-dihydrobenzo[b][1,4]dioxin-6-yl, 8-chloro-3,3-dimethyl-2,3-dihydrobenzo[b][1,4]dioxin-6-yl, 2,2,3,3-tetrafluoro-2,3-dihydrobenzo[b][1,4]dioxin-6-yl, 3,3-difluoro-2,3-dihydrobenzo[b][1,4]dioxin-6-yl, 7-fluoro-2,2-dimethyl-2,3-dihydrobenzo[b][1,4]dioxin-6-yl, or 7-chloro-3,3-dimethyl-2,3-dihydrobenzo[b][1,4]dioxin-6-yl; or
2,2-dimethylbenzo[d][1,3]dioxol-5-yl, 2,2-difluorobenzo[d][1,3]dioxol-5-yl, 2-ethyl-2-methylbenzo[d][1,3]dioxol-5-yl, spiro[benzo[d][1,3]dioxole-2,1′-cyclobutan]-5-yl, 6-chloro-2,2-dimethylbenzo[d][1,3]dioxol-5-yl, 7-chloro-2,2-dimethylbenzo[d][1,3]dioxol-5-yl, 4-chloro-2,2-dimethylbenzo[d][1,3]dioxol-5-yl, 7-fluoro-2,2-dimethylbenzo[d][1,3]dioxol-5-yl, 4-fluoro-2,2-dimethylbenzo[d][1,3]dioxol-5-yl, 4,6-difluoro-2,2-dimethylbenzo[d][1,3]dioxol-5-yl, 2,2,7-trimethylbenzo[d][1,3]dioxol-5-yl, 7-fluoro-2,2-dimethylbenzo[d][1,3]dioxol-4-yl, 2,2-dimethylbenzo[d][1,3]dioxol-4-yl, 2,2-difluorobenzo[d][1,3]dioxol-4-yl, 4-fluoro-2,2-dimethylbenzo[d][1,3]dioxol-5-yl, or 2,2-dimethylbenzo[d][1,3]dioxol-5-yl; or
2-(trifluoromethyl)pyridin-3-yl, 6-isopropoxypyridin-2-yl, 6-(trifluoromethyl)pyridin-2-yl, 2-(trifluoromethyl)pyridin-4-yl, 4-(trifluoromethyl)pyridin-2-yl, 2-(azetidin-1-yl)pyridin-4-yl, 2-(isopropylamino)pyridin-4-yl, 6-isopropylpyridin-2-yl, 2-isopropoxypyridin-4-yl, 6-(trifluoromethoxy)pyridin-2-yl, 2-(tert-butyl)pyridin-4-yl, 2-(methylcarbamoyl)pyridin-4-yl, 2-(2,2-dimethylcarbamoyl)pyridin-4-yl, 5,6-dimethylpyridin-2-yl, 2-phenylpyridin-4-yl, 5-(difluoromethyl)pyridin-2-yl, 5-methylpyridin-2-yl, 5-(trifluoromethyl)pyridin-2-yl, 2-methoxypyridin-4-yl, 1-methyl-6-oxo-1,6-dihydropyridin-3-yl, 1-methyl-2-oxo-1,2-dihydropyridin-4-yl, 2-(trifluoromethyl)thiazol-4-yl, or 1-methyl-5-(trifluoromethyl)-1H-pyrazol-4-yl; or
quinoxalin-6-yl, 3-methylquinoxalin-6-yl, 3-(trifluoromethyl)quinoxalin-6-yl, or quinoxalin-2-yl; or
isoquinolin-3-yl, 2-methyl-1-oxo-1,2-dihydroisoquinolin-7-yl, 2-methyl-1-oxo-1,2,3,4-tetrahydroisoquinolin-7-yl, 1,2-dimethyl-4-oxo-1,4-dihydroquinolin-7-yl, 1,3-dimethyl-4-oxo-1,4-dihydroquinolin-6-yl, or 2-methylquinolin-7-yl; or
2-methyl-1H-benzo[d]imidazol-5-yl, 2,2-dimethyl-2,3-dihydrobenzofuran-6-yl, 2,2-dimethyl-3-oxo-2,3-dihydrobenzofuran-6-yl, 3,3-dimethyl-2,3-dihydrobenzofuran-5-yl, or 3-methyl-4-oxo-3,4-dihydro-2H-benzo[e][1,3]oxazin-6-yl;
2,3-dihydro-[1,4]dioxino[2,3-b]pyridin-6-yl, 3,3-dimethyl-2,3-dihydro-[1,4]dioxino[2,3-b]pyridin-6-yl, 4,4-difluoro-3,4-dihydro-2H-pyrano[3,2-b]pyridin-6-yl, 4,4-difluoro-3,4-dihydro-2H-pyrano[3,2-b]pyridin-8-yl,
7-chloro-3,3-dimethyl-2,3-dihydro-[1,4]dioxino[2,3-b]pyridin-6-yl, 7-fluoro-3,3-dimethyl-2,3-dihydro-[1,4]dioxino[2,3-b]pyridin-6-yl, 8-fluoro-3,3-dimethyl-2,3-dihydro-[1,4]dioxino[2,3-b]pyridin-6-yl, 2,2-dimethyl-2,3-dihydrofuro[2,3-b]pyridin-6-yl, 3,3-dimethyl-2,3-dihydrofuro[3,2-b]pyridin-5-yl, 3,3-dimethyl-2,3-dihydrofuro[3,2-b]pyridine-5-yl, 2,2-dimethyl-2,3-dihydro-[1,4]dioxino[2,3-c]pyridin-7-yl, 2,2-dimethyl-2,3-dihydro-[1,4]dioxino[2,3-b]pyridin-7-yl, 3-methyl-2,3-dihydro-[1,4]dioxino[2,3-b]pyridin-6-yl, or 3,3-dimethyl-2,3-dihydro-[1,4]dioxino[2,3-b]pyridin-6-yl; or
2-methylbenzo[d]thiazol-5-yl, 2-methylbenzo[d]thiazol-6-yl, 2-ethylbenzo[d]thiazol-5-yl, 2-isopropylbenzo[d]thiazol-5-yl, or 2-(trifluoromethyl)thiazol-5-yl; or
2-methyl-1,3-dioxoisoindolin-5-yl, 2-isopropyl-1,3-dioxoisoindolin-5-yl, 1-oxo-2-(2,2,2-trifluoroethyl)isoindolin-5-yl, 2,3,3-trimethyl-1-oxoisoindolin-5-yl, 2-methyl-3-oxoisoindolin-5-yl, or 2-methyl-1-oxoisoindolin-5-yl.

27. The compound of claim 6, wherein the moiety 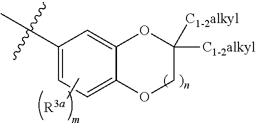
is 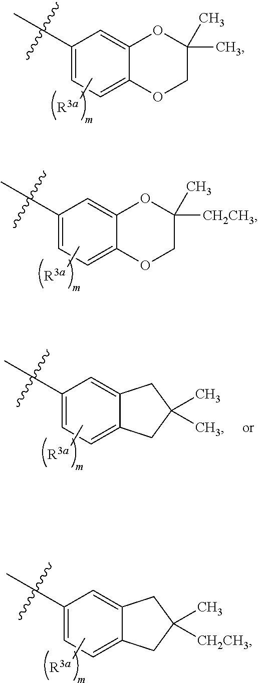
wherein m is 0, 1, 2 or 3 and R^{3a} is alkoxy, alkyl, halogen, and oxo.

28. The compound of claim 27, wherein the moiety 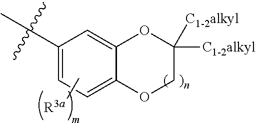
is 2,2-dimethylbenzo[d][1,3]dioxol-5-yl or 3,3-dimethyl-2,3-dihydrobenzo[b][1,4]dioxin-6-yl, wherein m is 0, 1, 2 or 3 and R^{3a} is alkoxy, alkyl, halogen, and oxo.

29. The compound of claim 27, wherein the moiety 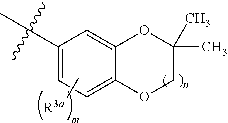
is 2,2-dimethylbenzo[d][1,3]dioxol-5-yl, 2,2-difluorobenzo[d][1,3]dioxol-5-yl, 2-ethyl-2-methylbenzo[d][1,3]dioxol-5-yl, spiro[benzo[d][1,3]dioxole-2,1′-cyclobutan]-5-yl, 6-chloro-2,2-dimethylbenzo[d][1,3]dioxol-5-yl, 7-chloro-2,2-dimethylbenzo[d][1,3]dioxol-5-yl, 4-chloro-2,2-dimethylbenzo[d][1,3]dioxol-5-yl, 7-fluoro-2,2-dimethylbenzo[d][1,3]dioxol-5-yl, 4-fluoro-2,2-dimethylbenzo[d][1,3]dioxol-5-yl, 4,6-difluoro-2,2-dimethylbenzo[d][1,3]dioxol-5-yl, 2,2,7-trimethylbenzo[d][1,3]dioxol-5-yl, 2,2-difluorobenzo[d][1,3]dioxol-4-yl, 4-fluoro-2,2-dimethylbenzo[d][1,3]dioxol-5-yl, 2,2-dimethylbenzo[d][1,3]dioxol-5-yl; 2,3-dihydrobenzo[b][1,4]dioxin-6-yl, 3,3-dimethyl-2,3-dihydrobenzo[b][1,4]dioxin-6-yl, 8-fluoro-3,3-dimethyl-2,3-dihydrobenzo[b][1,4]dioxin-6-yl, 7-fluoro-3,3-dimethyl-2,3-dihydrobenzo[b][1,4]dioxin-6-yl, 5-fluoro-3,3-dimethyl-2,3-dihydrobenzo[b][1,4]dioxin-6-yl, 8-chloro-3,3-dimethyl-2,3-dihydrobenzo[b][1,4]dioxin-6-yl, 2,2,3,3-tetrafluoro-2,3-dihydrobenzo[b][1,4]dioxin-6-yl, 3,3-difluoro-2,3-dihydrobenzo[b][1,4]dioxin-6-yl, 7-fluoro-2,2-dimethyl-2,3-dihydrobenzo[b][1,4]dioxin-6-yl, or 7-chloro-3,3-dimethyl-2,3-dihydrobenzo[b][1,4]dioxin-6-yl.

30. The compound of claim 1, wherein the compound is selected from:
2-(7-((2S,5R)-2,5-dimethyl-4-(1-(quinoxalin-6-yl)ethyl)piperazin-1-yl)-3,4-dimethyl-5-oxo-4,5-dihydro-3H-imidazo[4,5-b]pyridin-2-yl)acetonitrile (A1);
2-(7-((2S,5R)-4-(1-(4-fluoro-2-methoxyphenyl)ethyl)-2,5-dimethylpiperazin-1-yl)-3,4-dimethyl-5-oxo-4,5-dihydro-3H-imidazo[4,5-b]pyridin-2-yl)acetonitrile (A4);
2-(7-((2S,5R)-2,5-dimethyl-4-(1-(p-tolyl)ethyl)piperazin-1-yl)-3,4-dimethyl-5-oxo-4,5-dihydro-3H-imidazo[4,5-b]pyridin-2-yl)acetonitrile (A5);
2-(7-((2S,5R)-2,5-dimethyl-4-(1-(1-methyl-5-(trifluoromethyl)-1H-pyrazol-4-yl)ethyl)piperazin-1-yl)-3,4-dimethyl-5-oxo-4,5-dihydro-3H-imidazo[4,5-b]pyridin-2-yl)acetonitrile (A6);
2-(7-((2S,5R)-2,5-dimethyl-4-(1-(2-(trifluoromethyl)pyridin-3-yl)ethyl)piperazin-1-yl)-3,4-dimethyl-5-oxo-4,5-dihydro-3H-imidazo[4,5-b]pyridin-2-yl)acetonitrile (A7);
2-(7-((2S,5R)-4-(1-(4-fluoro-2-(trifluoromethyl)phenyl)ethyl)-2,5-dimethylpiperazin-1-yl)-4-methyl-5-oxo-4,5-dihydro-3H-imidazo[4,5-b]pyridin-2-yl)acetonitrile (A9);
2-(6-((2R,5R)-4-(1-(4-fluoro-2-(trifluoromethyl)phenyl)ethyl)-2-(methoxymethyl)-5-methylpiperazin-1-yl)-3,9-dimethyl-2-oxo-3,9-dihydro-2H-purin-8-yl)acetonitrile (A10);
2-(6-((2R,5R)-4-(1-(2,3-dihydrobenzo[b][1,4]dioxin-6-yl)ethyl)-2-(methoxymethyl)-5-methylpiperazin-1-yl)-3,9-dimethyl-2-oxo-3,9-dihydro-2H-purin-8-yl)acetonitrile (A11);
2-(6-((2R,5R)-4-(1-(3,3-dimethyl-2,3-dihydrobenzo[b][1,4]dioxin-6-yl)ethyl)-2-(methoxymethyl)-5-methylpiperazin-1-yl)-3,9-dimethyl-2-oxo-3,9-dihydro-2H-purin-8-yl)acetonitrile (A12);
2-(6-((2R,5R)-2-(methoxymethyl)-5-methyl-4-(1-(3-methylquinoxalin-6-yl)ethyl)piperazin-1-yl)-3,9-dimethyl-2-oxo-3,9-dihydro-2H-purin-8-yl)acetonitrile (A13);
2-(6-fluoro-7-((2S,5R)-4-(1-(4-fluoro-2-(trifluoromethyl)phenyl)ethyl)-2,5-dimethylpiperazin-1-yl)-3,4-dimethyl-5-oxo-4,5-dihydro-3H-imidazo[4,5-b]pyridin-2-yl)acetonitrile (A14);
2-(6-fluoro-7-((2S,5R)-4-(1-(4-fluoro-2-methoxyphenyl)ethyl)-2,5-dimethylpiperazin-1-yl)-3,4-dimethyl-5-oxo-4,5-dihydro-3H-imidazo[4,5-b]pyridin-2-yl)acetonitrile (A16);
2-(7-((2S,5R)-4-(1-(2,3-dihydrobenzo[b][1,4]dioxin-6-yl)ethyl)-2,5-dimethylpiperazin-1-yl)-6-fluoro-3,4-dimethyl-5-oxo-4,5-dihydro-3H-imidazo[4,5-b]pyridin-2-yl)acetonitrile (A18);
2-(7-((2S,5R)-4-(1-(3,3-dimethyl-2,3-dihydrobenzo[b][1,4]dioxin-6-yl)ethyl)-2,5-dimethylpiperazin-1-yl)-6-fluoro-3,4-dimethyl-5-oxo-4,5-dihydro-3H-imidazo[4,5-b]pyridin-2-yl)acetonitrile (A19);
2-(7-((2S,5R)-4-(1-(3,3-dimethyl-2,3-dihydrobenzo[b][1,4]dioxin-6-yl)ethyl)-2,5-dimethylpiperazin-1-yl)-3-ethyl-6-fluoro-4-methyl-5-oxo-4,5-dihydro-3H-imidazo[4,5-b]pyridin-2-yl)acetonitrile (A20);
2-(7-((2S,5R)-4-(1-(3,3-dimethyl-2,3-dihydro-[1,4]dioxino[2,3-b]pyridin-6-yl)ethyl)-2,5-dimethylpiperazin-1-yl)-3-ethyl-6-fluoro-4-methyl-5-oxo-4,5-dihydro-3H-imidazo[4,5-b]pyridin-2-yl)acetonitrile (A21);
2-(7-((2S,5R)-2,5-dimethyl-4-(1-(quinoxalin-6-yl)ethyl)piperazin-1-yl)-6-fluoro-3,4-dimethyl-5-oxo-4,5-dihydro-3H-imidazo[4,5-b]pyridin-2-yl)acetonitrile (A22);
2-(7-((2S,5R)-2,5-dimethyl-4-(1-(3-methylquinoxalin-6-yl)ethyl)piperazin-1-yl)-6-fluoro-3,4-dimethyl-5-oxo-4,5-dihydro-3H-imidazo[4,5-b]pyridin-2-yl)acetonitrile (A23);
2-(6-((2S,5R)-4-(1-(4-fluoro-2-methoxyphenyl)ethyl)-2,5-dimethylpiperazin-1-yl)-3,9-dimethyl-2-oxo-3,9-dihydro-2H-purin-8-yl)acetonitrile (A24);
2-(6-((2S,5R)-4-(1-(2-(difluoromethoxy)-4-fluorophenyl)ethyl)-2,5-dimethylpiperazin-1-yl)-3,9-dimethyl-2-oxo-3,9-dihydro-2H-purin-8-yl)acetonitrile (A25);
2-(6-((2S,5R)-4-(1-(4-fluoro-2-(trifluoromethoxy)phenyl)ethyl)-2,5-dimethylpiperazin-1-yl)-3,9-dimethyl-2-oxo-3,9-dihydro-2H-purin-8-yl)acetonitrile (A26);
2-(6-((2S,5R)-4-(1-(4-fluoro-2-(methoxymethyl)phenyl)ethyl)-2,5-dimethylpiperazin-1-yl)-3,9-dimethyl-2-oxo-3,9-dihydro-2H-purin-8-yl)acetonitrile (A27);
2-(6-((2S,5R)-4-(1-(4-fluoro-2-(1-methoxyethyl)phenyl)ethyl)-2,5-dimethylpiperazin-1-yl)-3,9-dimethyl-2-oxo-3,9-dihydro-2H-purin-8-yl)acetonitrile (A28);
2-(6-((2S,5R)-4-(1-(2-(1-(difluoromethoxy)ethyl)-4-fluorophenyl)ethyl)-2,5-dimethylpiperazin-1-yl)-3,9-dimethyl-2-oxo-3,9-dihydro-2H-purin-8-yl)acetonitrile (A29);
2-(6-((2S,5R)-4-(1-(2-(difluoromethyl)-4-fluorophenyl)ethyl)-2,5-dimethylpiperazin-1-yl)-3,9-dimethyl-2-oxo-3,9-dihydro-2H-purin-8-yl)acetonitrile (A30);
2-(6-((2S,5R)-4-(1-(2-(1,1-difluoroethoxy)-4-fluorophenyl)ethyl)-2,5-dimethylpiperazin-1-yl)-3,9-dimethyl-2-oxo-3,9-dihydro-2H-purin-8-yl)acetonitrile (A31);
2-(6-((2S,5R)-4-(1-(4-fluoro-2-(trifluoromethyl)phenyl)ethyl)-2,5-dimethylpiperazin-1-yl)-3,9-dimethyl-2-oxo-3,9-dihydro-2H-purin-8-yl)acetonitrile (A33);
2-(6-((2S,5R)-2,5-diethyl-4-(1-(4-fluoro-2-(trifluoromethyl)phenyl)ethyl)piperazin-1-yl)-3,9-dimethyl-2-oxo-3,9-dihydro-2H-purin-8-yl)acetonitrile (A34);
2-(9-ethyl-6-((2S,5R)-4-(1-(4-fluoro-2-(trifluoromethyl)phenyl)ethyl)-2,5-dimethylpiperazin-1-yl)-3-methyl-2-oxo-3,9-dihydro-2H-purin-8-yl)acetonitrile (A35);
2-(6-((2S,5R)-4-(1-(4-fluoro-2-(trifluoromethyl)phenyl)ethyl)-2,5-dimethylpiperazin-1-yl)-3,9-bis(methyl-d3)-2-oxo-3,9-dihydro-2H-purin-8-yl)acetonitrile (A36);
2-(6-((2S,5R)-4-(1-(4-fluoro-2-(trifluoromethyl)phenyl)ethyl)-2,5-dimethylpiperazin-1-yl)-3-methyl-9-(methyl-d3)-2-oxo-3,9-dihydro-2H-purin-8-yl)acetonitrile (A37);
2-(6-((2S,5R)-2,5-dimethyl-4-(1-(3-(trifluoromethyl)phenyl)ethyl)piperazin-1-yl)-3,9-dimethyl-2-oxo-3,9-dihydro-2H-purin-8-yl)acetonitrile (A41);
2-(6-((2S,5R)-2,5-dimethyl-4-(1-(3-(trifluoromethyl)phenyl)ethyl)piperazin-1-yl)-9-ethyl-3-methyl-2-oxo-3,9-dihydro-2H-purin-8-yl)acetonitrile (A42);
2-(9-ethyl-6-((2S,5R)-4-(1-(3-isopropoxyphenyl)ethyl)-2,5-dimethylpiperazin-1-yl)-3-methyl-2-oxo-3,9-dihydro-2H-purin-8-yl)acetonitrile (A43);
2-(6-((2S,5R)-2,5-dimethyl-4-(1-(3-(trifluoromethoxy)phenyl)ethyl)piperazin-1-yl)9-ethyl-3-methyl-2-oxo-3,9-dihydro-2H-purin-8-yl)acetonitrile (A44);
2-(6-((2S,5R)-2,5-dimethyl-4-(1-(quinoxalin-6-yl)ethyl)piperazin-1-yl)-3,9-dimethyl-2-oxo-3,9-dihydro-2H-purin-8-yl)acetonitrile (A48);
2-(6-((2S,5R)-2,5-dimethyl-4-(1-(3-methylquinoxalin-6-yl)ethyl)piperazin-1-yl)-3,9-dimethyl-2-oxo-3,9-dihydro-2H-purin-8-yl)acetonitrile (A49);
2-(6-((2S,5R)-5-ethyl-2-methyl-4-(1-(3-methylquinoxalin-6-yl)ethyl)piperazin-1-yl)-3,9-dimethyl-2-oxo-3,9-dihydro-2H-purin-8-yl)acetonitrile (A50);
2-(6-((2S,5R)-2,5-diethyl-4-(1-(3-methylquinoxalin-6-yl)ethyl)piperazin-1-yl)-3,9-dimethyl-2-oxo-3,9-dihydro-2H-purin-8-yl)acetonitrile (A51);
2-(6-((2S,5R)-2,5-dimethyl-4-(1-(3-methylquinoxalin-6-yl)ethyl)piperazin-1-yl)-9-ethyl-3-methyl-2-oxo-3,9-dihydro-2H-purin-8-yl)acetonitrile (A52);
2-(6-((2S,5R)-2,5-dimethyl-4-(1-(3-methylquinoxalin-6-yl)ethyl)piperazin-1-yl)-3-ethyl-9-methyl-2-oxo-3,9-dihydro-2H-purin-8-yl)acetonitrile (A53);
2-(6-((2S,5R)-2,5-dimethyl-4-(1-(3-(trifluoromethyl)quinoxalin-6-yl)ethyl)piperazin-1-yl)-9-ethyl-3-methyl-2-oxo-3,9-dihydro-2H-purin-8-yl)acetonitrile (A54);
2-(6-((2S,5R)-2,5-dimethyl-4-(1-(2-methylbenzo[d]thiazol-5-yl)ethyl)piperazin-1-yl)-9-ethyl-3-methyl-2-oxo-3,9-dihydro-2H-purin-8-yl)acetonitrile (A55);
2-(6-((2S,5R)-2,5-dimethyl-4-(1-(2-methylbenzo[d]thiazol-6-yl)ethyl)piperazin-1-yl)-9-ethyl-3-methyl-2-oxo-3,9-dihydro-2H-purin-8-yl)acetonitrile (A56);
2-(6-((2S,5R)-2,5-dimethyl-4-(1-(2-methyl-1H-benzo[d]imidazol-5-yl)ethyl)piperazin-1-yl)-9-ethyl-3-methyl-2-oxo-3,9-dihydro-2H-purin-8-yl)acetonitrile (A57);
2-(6-((2S,5R)-4-(1-(2,3-dihydrobenzo[b][1,4]dioxin-6-yl)ethyl)-2,5-dimethylpiperazin-1-yl)-3,9-dimethyl-2-oxo-3,9-dihydro-2H-purin-8-yl)acetonitrile (A58);
2-(6-((2S,5R)-4-(1-(3,3-dimethyl-2,3-dihydrobenzo[b][1,4]dioxin-6-yl)ethyl)-2,5-dimethylpiperazin-1-yl)-3,9-dimethyl-2-oxo-3,9-dihydro-2H-purin-8-yl)acetonitrile (A59);
2-(6-((2S,5R)-4-(1-(3,3-dimethyl-2,3-dihydrobenzo[b][1,4]dioxin-6-yl)ethyl)-2,5-dimethylpiperazin-1-yl)-9-ethyl-3-methyl-2-oxo-3,9-dihydro-2H-purin-8-yl)acetonitrile (A60);
2-(6-((2S,5R)-4-(1-(3,3-dimethyl-2,3-dihydrobenzo[b][1,4]dioxin-6-yl)ethyl)-2,5-dimethylpiperazin-1-yl)-3-ethyl-9-methyl-2-oxo-3,9-dihydro-2H-purin-8-yl)acetonitrile (A61);
2-(6-((2S,5R)-4-(1-(3,3-dimethyl-2,3-dihydrobenzo[b][1,4]dioxin-6-yl)ethyl)-2,5-dimethylpiperazin-1-yl)-3,9-diethyl-2-oxo-3,9-dihydro-2H-purin-8-yl)acetonitrile (A62);
2-(6-((2S,5R)-4-(1-(3,3-dimethyl-2,3-dihydrobenzo[b][1,4]dioxin-6-yl)ethyl)-5-ethyl-2-methylpiperazin-1-yl)-3,9-dimethyl-2-oxo-3,9-dihydro-2H-purin-8-yl)acetonitrile (A63);
2-(6-((2S,5R)-4-(1-(3,3-dimethyl-2,3-dihydrobenzo[b][1,4]dioxin-6-yl)ethyl)-2,5-diethylpiperazin-1-yl)-3,9-dimethyl-2-oxo-3,9-dihydro-2H-purin-8-yl)acetonitrile (A64);
2-(6-((2S,5R)-4-(1-(3,3-dimethyl-2,3-dihydrobenzo[b][1,4]dioxin-6-yl)ethyl)-5-ethyl-2-methylpiperazin-1-yl)-9-ethyl-3-methyl-2-oxo-3,9-dihydro-2H-purin-8-yl)acetonitrile (A65);
2-(6-((2S,5R)-4-(1-(3,3-dimethyl-2,3-dihydrobenzo[b][1,4]dioxin-6-yl)ethyl)-5-ethyl-2-methylpiperazin-1-yl)-3-ethyl-9-methyl-2-oxo-3,9-dihydro-2H-purin-8-yl)acetonitrile (A66);
2-(6-((2S,5R)-4-(1-(3,3-dimethyl-2,3-dihydrobenzo[b][1,4]dioxin-6-yl)ethyl)-2,5-diethylpiperazin-1-yl)-9-ethyl-3-methyl-2-oxo-3,9-dihydro-2H-purin-8-yl)acetonitrile (A67);
2-(6-((2S,5R)-4-(1-(8-fluoro-3,3-dimethyl-2,3-dihydrobenzo[b][1,4]dioxin-6-yl)ethyl)-2,5-dimethylpiperazin-1-yl)-3,9-dimethyl-2-oxo-3,9-dihydro-2H-purin-8-yl)acetonitrile (A68);
2-(6-((2S,5R)-4-(1-(7-fluoro-3,3-dimethyl-2,3-dihydrobenzo[b][1,4]dioxin-6-yl)ethyl)-2,5-dimethylpiperazin-1-yl)-3,9-dimethyl-2-oxo-3,4,5,9-tetrahydro-2H-purin-8-yl)acetonitrile (A69);
2-(6-((2S,5R)-4-(1-(5-fluoro-3,3-dimethyl-2,3-dihydrobenzo[b][1,4]dioxin-6-yl)ethyl)-2,5-dimethylpiperazin-1-yl)-3,9-dimethyl-2-oxo-3,9-dihydro-2H-purin-8-yl)acetonitrile (A70);
2-(9-ethyl-6-((2S,5R)-4-(1-(5-fluoro-3,3-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile (A71);
2-(6-((2S,5R)-4-(1-(8-chloro-3,3-dimethyl-2,3-dihydrobenzo[b][1,4]dioxin-6-yl)ethyl)-2,5-dimethylpiperazin-1-yl)-9-ethyl-3-methyl-2-oxo-3,9-dihydro-2H-purin-8-yl)acetonitrile (A72);
2-(6-((2S,5R)-4-(1-(3,3-dimethyl-2,3-dihydro-[1,4]dioxino[2,3-b]pyridin-6-yl)ethyl)-2,5-dimethylpiperazin-1-yl)-9-ethyl-3-methyl-2-oxo-3,9-dihydro-2H-purin-8-yl)acetonitrile (A74);
2-(6-((2S,5R)-4-(1-(4,4-difluorochroman-6-yl)ethyl)-2,5-dimethylpiperazin-1-yl)-3,9-dimethyl-2-oxo-3,9-dihydro-2H-purin-8-yl)acetonitrile (A77);
2-(6-((2S,5R)-4-(1-(4,4-difluorochroman-6-yl)ethyl)-2,5-dimethylpiperazin-1-yl)-9-ethyl-3-methyl-2-oxo-3,9-dihydro-2H-purin-8-yl)acetonitrile (A78);
2-(6-((2S,5R)-2,5-dimethyl-4-(1-(2,2,3,3-tetrafluoro-2,3-dihydrobenzo[b][1,4]dioxin-6-yl)ethyl)piperazin-1-yl)-3,9-dimethyl-2-oxo-3,9-dihydro-2H-purin-8-yl)acetonitrile (A79);
2-(6-((2S,5R)-4-(1-(3,3-difluoro-2,3-dihydrobenzo[b][1,4]dioxin-6-yl)ethyl)-2,5-dimethylpiperazin-1-yl)-9-ethyl-3-methyl-2-oxo-3,9-dihydro-2H-purin-8-yl)acetonitrile (A80);
2-(6-((2S,5R)-4-(1-(2,2-dimethyl-4-oxochroman-7-yl)ethyl)-2,5-dimethylpiperazin-1-yl)-9-ethyl-3-methyl-2-oxo-3,9-dihydro-2H-purin-8-yl)acetonitrile (A81);
2-(6-((2S,5R)-2,5-dimethyl-4-(1-(2-methyl-1,3-dioxoisoindolin-5-yl)ethyl)piperazin-1-yl)-9-ethyl-3-methyl-2-oxo-3,9-dihydro-2H-purin-8-yl)acetonitrile (A83);
2-(6-((2S,5R)-4-(1-(2,2-dimethylbenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile (A85);
2-(9-cyclopropyl-6-((2S,5R)-4-(1-(3,3-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile (A86);
2-(9-(2,2-difluoroethyl)-6-((2S,5R)-4-(1-(3,3-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile (A87);
2-(6-((2S,5R)-4-(1-(3,3-dimethyl-2,3-dihydrobenzo[b][1,4]dioxin-6-yl)ethyl)-2,5-dimethylpiperazin-1-yl)-3-methyl-2-oxo-9-propyl-3,9-dihydro-2H-purin-8-yl)acetonitrile (A88);
2-(6-((2S,5R)-2,5-dimethyl-4-(1-(3-methyl-4-oxo-4H-chromen-6-yl)ethyl)piperazin-1-yl)-9-ethyl-3-methyl-2-oxo-3,9-dihydro-2H-purin-8-yl)acetonitrile (A91);
2-(6-((2S,5R)-4-(1-(2,2-dimethylchroman-7-yl)ethyl)-2,5-dimethylpiperazin-1-yl)-9-ethyl-3-methyl-2-oxo-3,9-dihydro-2H-purin-8-yl)acetonitrile (A94);
2-(9-ethyl-6-((2S,5R)-4-(1-(2-ethylbenzo[d]thiazol-5-yl)ethyl)-2,5-dimethylpiperazin-1-yl)-3-methyl-2-oxo-3,9-dihydro-2H-purin-8-yl)acetonitrile (A95);
2-(9-ethyl-6-((2S,5R)-4-(1-(2-isopropylbenzo[d]thiazol-5-yl)ethyl)-2,5-dimethylpiperazin-1-yl)-3-methyl-2-oxo-3,9-dihydro-2H-purin-8-yl)acetonitrile (A96);
2-(6-((2S,5R)-4-(1-(2,2-dimethyl-2,3-dihydrobenzofuran-6-yl)ethyl)-2,5-dimethylpiperazin-1-yl)-9-ethyl-3-methyl-2-oxo-3,9-dihydro-2H-purin-8-yl)acetonitrile (A97);
2-(6-((2S,5R)-4-(1-(2,2-dimethylbenzo[d][1,3]dioxol-5-yl)ethyl)-2,5-dimethylpiperazin-1-yl)-3,9-dimethyl-2-oxo-3,9-dihydro-2H-purin-8-yl)acetonitrile (A98);
2-(6-((2S,5R)-4-(1-(2,2-difluorobenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile (A99);
2-(6-((2S,5R)-4-(1-(4,4-difluoro-3,4-dihydro-2H-pyrano[3,2-b]pyridin-6-yl)ethyl)-2,5-dimethylpiperazin-1-yl)-9-ethyl-3-methyl-2-oxo-3,9-dihydro-2H-purin-8-yl)acetonitrile (A102);
2-(6-((2S,5R)-4-(1-(4,4-difluoro-3,4-dihydro-2H-pyrano[3,2-b]pyridin-8-yl)ethyl)-2,5-dimethylpiperazin-1-yl)-9-ethyl-3-methyl-2-oxo-3,9-dihydro-2H-purin-8-yl)acetonitrile (A103);
2-(9-ethyl-6-((2S,5R)-4-(1-(3-isopropylphenyl)ethyl)-2,5-dimethylpiperazin-1-yl)-3-methyl-2-oxo-3,9-dihydro-2H-purin-8-yl)acetonitrile (A108);
2-(6-((2S,5R)-4-(1-(3-(tert-butyl)phenyl)ethyl)-2,5-dimethylpiperazin-1-yl)-9-ethyl-3-methyl-2-oxo-3,9-dihydro-2H-purin-8-yl)acetonitrile (A110);
2-(9-ethyl-6-((2S,5R)-4-(1-(3-(2-hydroxypropan-2-yl)phenyl)ethyl)-2,5-dimethylpiperazin-1-yl)-3-methyl-2-oxo-3,9-dihydro-2H-purin-8-yl)acetonitrile (A111);
2-(6-((2S,5R)-4-(1-(3-(difluoromethyl)-4-fluorophenyl)ethyl)-2,5-dimethylpiperazin-1-yl)-9-ethyl-3-methyl-2-oxo-3,9-dihydro-2H-purin-8-yl)acetonitrile (A112);
2-(9-ethyl-6-((2S,5R)-4-(1-(4-fluoro-3-(trifluoromethyl)phenyl)ethyl)-2,5-dimethylpiperazin-1-yl)-3-methyl-2-oxo-3,9-dihydro-2H-purin-8-yl)acetonitrile (A113);
2-(9-ethyl-6-((2S,5R)-4-(1-(2-fluoro-5-(trifluoromethyl)phenyl)ethyl)-2,5-dimethylpiperazin-1-yl)-3-methyl-2-oxo-3,9-dihydro-2H-purin-8-yl)acetonitrile (A114);
2-(6-((2S,5R)-2,5-dimethyl-4-(1-(4-methyl-3-(trifluoromethyl)phenyl)ethyl)piperazin-1-yl)-9-ethyl-3-methyl-2-oxo-3,9-dihydro-2H-purin-8-yl)acetonitrile (A115);
2-(6-((2S,5R)-2,5-dimethyl-4-(1-(4-methyl-2-(trifluoromethyl)phenyl)ethyl)piperazin-1-yl)-9-ethyl-3-methyl-2-oxo-3,9-dihydro-2H-purin-8-yl)acetonitrile (A116);
2-(9-ethyl-6-((2S,5R)-4-(1-(6-isopropoxypyridin-2-yl)ethyl)-2,5-dimethylpiperazin-1-yl)-3-methyl-2-oxo-3,9-dihydro-2H-purin-8-yl)acetonitrile (A117);
2-(6-((2S,5R)-2,5-dimethyl-4-(1-(6-(trifluoromethyl)pyridin-2-yl)ethyl)piperazin-1-yl)-9-ethyl-3-methyl-2-oxo-3,9-dihydro-2H-purin-8-yl)acetonitrile (A118);
2-(6-((2S,5R)-2,5-dimethyl-4-(1-(2-(trifluoromethyl)pyridin-4-yl)ethyl)piperazin-1-yl)-9-ethyl-3-methyl-2-oxo-3,9-dihydro-2H-purin-8-yl)acetonitrile (A119);
2-(6-((2S,5R)-2,5-dimethyl-4-(1-(4-(trifluoromethyl)pyridin-2-yl)ethyl)piperazin-1-yl)-9-ethyl-3-methyl-2-oxo-3,9-dihydro-2H-purin-8-yl)acetonitrile (A120);
2-(6-((2S,5R)-4-(1-(2-(azetidin-1-yl)pyridin-4-yl)ethyl)-2,5-dimethylpiperazin-1-yl)-9-ethyl-3-methyl-2-oxo-3,9-dihydro-2H-purin-8-yl)acetonitrile (A121);
2-(9-ethyl-6-((2S,5R)-4-(1-(2-(isopropylamino)pyridin-4-yl)ethyl)-2,5-dimethylpiperazin-1-yl)-3-methyl-2-oxo-3,9-dihydro-2H-purin-8-yl)acetonitrile (A122);
2-(9-ethyl-6-((2S,5R)-4-(1-(6-isopropylpyridin-2-yl)ethyl)-2,5-dimethylpiperazin-1-yl)-3-methyl-2-oxo-3,9-dihydro-2H-purin-8-yl)acetonitrile (A123);
2-(9-ethyl-6-((2S,5R)-4-(1-(2-isopropoxypyridin-4-yl)ethyl)-2,5-dimethylpiperazin-1-yl)-3-methyl-2-oxo-3,9-dihydro-2H-purin-8-yl)acetonitrile (A124);
2-(6-((2S,5R)-4-(1-(3-cyclopropylphenyl)ethyl)-2,5-dimethylpiperazin-1-yl)-9-ethyl-3-methyl-2-oxo-3,9-dihydro-2H-purin-8-yl)acetonitrile (A125);
2-(6-((2S,5R)-2,5-dimethyl-4-(1-(3-(1-methylcyclopropyl)phenyl)ethyl)piperazin-1-yl)-9-ethyl-3-methyl-2-oxo-3,9-dihydro-2H-purin-8-yl)acetonitrile (A126);
2-(6-((2S,5R)-2,5-dimethyl-4-(1-(3-(oxetan-3-yl)phenyl)ethyl)piperazin-1-yl)-9-ethyl-3-methyl-2-oxo-3,9-dihydro-2H-purin-8-yl)acetonitrile (A127);
2-(6-((2S,5R)-2,5-dimethyl-4-(1-(6-(trifluoromethoxy)pyridin-2-yl)ethyl)piperazin-1-yl)-9-ethyl-3-methyl-2-oxo-3,9-dihydro-2H-purin-8-yl)acetonitrile (A128);
2-(6-((2S,5R)-4-(1-(2-(tert-butyl)pyridin-4-yl)ethyl)-2,5-dimethylpiperazin-1-yl)-9-ethyl-3-methyl-2-oxo-3,9-dihydro-2H-purin-8-yl)acetonitrile (A129);
4-(1-((2R,5S)-4-(8-(cyanomethyl)-9-ethyl-3-methyl-2-oxo-3,9-dihydro-2H-purin-6-yl)-2,5-dimethylpiperazin-1-yl)ethyl)-N-methylpicolinamide (A130);
4-(1-((2R,5S)-4-(8-(cyanomethyl)-9-ethyl-3-methyl-2-oxo-3,9-dihydro-2H-purin-6-yl)-2,5-dimethylpiperazin-1-yl)ethyl)-N,N-dimethylpicolinamide (A131);
5-(1-((2R,5S)-4-(8-(cyanomethyl)-9-ethyl-3-methyl-2-oxo-3,9-dihydro-2H-purin-6-yl)-2,5-dimethylpiperazin-1-yl)ethyl)-2-fluoro-N-methylbenzamide (A132);
2-(9-ethyl-6-((2S,5R)-4-(1-(3-(1-hydroxy-2-methylpropan-2-yl)phenyl)ethyl)-2,5-dimethylpiperazin-1-yl)-3-methyl-2-oxo-3,9-dihydro-2H-purin-8-yl)acetonitrile (A133);
2-(3-(1-((2R,5S)-4-(8-(cyanomethyl)-9-ethyl-3-methyl-2-oxo-3,9-dihydro-2H-purin-6-yl)-2,5-dimethylpiperazin-1-yl)ethyl)phenyl)-2-methylpropanenitrile (A134);
2-(9-ethyl-6-((2S,5R)-4-(1-(4-fluoro-3-(prop-1-en-2-yl)phenyl)ethyl)-2,5-dimethylpiperazin-1-yl)-3-methyl-2-oxo-3,9-dihydro-2H-purin-8-yl)acetonitrile (A135);
2-(9-ethyl-6-((2S,5R)-4-(1-(4-fluoro-3-(1-methylcyclopropyl)phenyl)ethyl)-2,5-dimethylpiperazin-1-yl)-3-methyl-2-oxo-3,9-dihydro-2H-purin-8-yl)acetonitrile (A136);
2-(6-((2S,5R)-2,5-dimethyl-4-(1-(3-methyl-4-(trifluoromethyl)phenyl)ethyl)piperazin-1-yl)-9-ethyl-3-methyl-2-oxo-3,9-dihydro-2H-purin-8-yl)acetonitrile (A137);
2-(6-((2S,5R)-4-(1-(5,6-dimethylpyridin-2-yl)ethyl)-2,5-dimethylpiperazin-1-yl)-9-ethyl-3-methyl-2-oxo-3,9-dihydro-2H-purin-8-yl)acetonitrile (A138);
2-(6-((2S,5R)-2,5-dimethyl-4-(1-(4-(morpholinomethyl)phenyl)ethyl)piperazin-1-yl)-9-ethyl-3-methyl-2-oxo-3,9-dihydro-2H-purin-8-yl)acetonitrile (A139);
2-(6-((2S,5R)-4-(1-(3-(2-oxa-6-azaspiro[3.3]heptan-6-yl)phenyl)ethyl)-2,5-dimethylpiperazin-1-yl)-9-ethyl-3-methyl-2-oxo-3,9-dihydro-2H-purin-8-yl)acetonitrile (A140);
2-(6-((2S,5R)-2,5-dimethyl-4-(1-(3-morpholinophenyl)ethyl)piperazin-1-yl)-9-ethyl-3-methyl-2-oxo-3,9-dihydro-2H-purin-8-yl)acetonitrile (A141);
2-(6-((2S,5R)-2,5-dimethyl-4-(1-(3-(morpholinomethyl)phenyl)ethyl)piperazin-1-yl)-9-ethyl-3-methyl-2-oxo-3,9-dihydro-2H-purin-8-yl)acetonitrile (A142);
2-(6-((2S,5R)-2,5-dimethyl-4-(1-(4-morpholinophenyl)ethyl)piperazin-1-yl)-9-ethyl-3-methyl-2-oxo-3,9-dihydro-2H-purin-8-yl)acetonitrile (A143);
2-(6-((2S,5R)-2,5-dimethyl-4-(1-(2-phenylpyridin-4-yl)ethyl)piperazin-1-yl)-9-ethyl-3-methyl-2-oxo-3,9-dihydro-2H-purin-8-yl)acetonitrile (A144);
2-(6-((2S,5R)-2,5-dimethyl-4-(1-(p-tolyl)but-2-yn-1-yl)piperazin-1-yl)-9-ethyl-3-methyl-2-oxo-3,9-dihydro-2H-purin-8-yl)acetonitrile (A145);
2-(6-((2S,5R)-4-(1-(5-(difluoromethyl)pyridin-2-yl)ethyl)-2,5-dimethylpiperazin-1-yl)-3,9-dimethyl-2-oxo-3,9-dihydro-2H-purin-8-yl)acetonitrile (A146);
2-(6-((2S,5R)-2,5-dimethyl-4-(1-(5-methylpyridin-2-yl)ethyl)piperazin-1-yl)-3,9-dimethyl-2-oxo-3,9-dihydro-2H-purin-8-yl)acetonitrile (A147);
2-(6-((2S,5R)-2,5-dimethyl-4-(1-(5-(trifluoromethyl)pyridin-2-yl)ethyl)piperazin-1-yl)-3,9-dimethyl-2-oxo-3,9-dihydro-2H-purin-8-yl)acetonitrile (A148);
2-(6-((2S,5R)-2,5-diethyl-4-(1-(4-(trifluoromethyl)phenyl)ethyl)piperazin-1-yl)-3,9-dimethyl-2-oxo-3,9-dihydro-2H-purin-8-yl)acetonitrile (A149);
2-(6-((2S,5R)-2,5-dimethyl-4-(1-(4-(trifluoromethyl)phenyl)ethyl)piperazin-1-yl)-3-methyl-2-oxo-3,9-dihydro-2H-purin-8-yl)acetonitrile (A150);
2-(6-((2S,5R)-2,5-diethyl-4-(1-(4-(trifluoromethyl)phenyl)ethyl)piperazin-1-yl)-3-methyl-2-oxo-3,9-dihydro-2H-purin-8-yl)acetonitrile (A151);
8-(but-2-yn-1-yl)-6-((2S,5R)-2,5-diethyl-4-(1-(4-(trifluoromethyl)phenyl)ethyl)piperazin-1-yl)-3,9-dimethyl-3,9-dihydro-2H-purin-2-one (A152);
2-(6-((2S,5R)-2,5-dimethyl-4-(1-(quinoxalin-2-yl)ethyl)piperazin-1-yl)-3,9-dimethyl-2-oxo-3,9-dihydro-2H-purin-8-yl)acetonitrile (A153);
2-(6-((2S,5R)-4-(1-(isoquinolin-3-yl)ethyl)-2,5-dimethylpiperazin-1-yl)-3,9-dimethyl-2-oxo-3,9-dihydro-2H-purin-8-yl)acetonitrile (A154);
2-(6-((2S,5R)-4-(1-(1,2-dimethyl-4-oxo-1,4-dihydroquinolin-7-yl)ethyl)-2,5-dimethylpiperazin-1-yl)-9-ethyl-3-methyl-2-oxo-3,9-dihydro-2H-purin-8-yl)acetonitrile (A155);
2-(9-ethyl-6-((2S,5R)-4-(1-(7-fluoro-2,2-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile (A156);
2-(9-ethyl-6-((2S,5R)-4-(1-(7-fluoro-3,3-dimethyl-2,3-dihydrobenzo[b][1,4]dioxin-6-yl)ethyl)-2,5-dimethylpiperazin-1-yl)-3-methyl-2-oxo-3,9-dihydro-2H-purin-8-yl)acetonitrile (A157);
2-(6-((2S,5R)-4-(1-(3,3-dimethyl-2,3-dihydro-[1,4]dioxino[2,3-b]pyridin-6-yl)ethyl)-5-ethyl-2-methylpiperazin-1-yl)-9-ethyl-3-methyl-2-oxo-3,9-dihydro-2H-purin-8-yl)acetonitrile (A158);
2-(6-((2S,5R)-4-(1-(3,3-dimethyl-2,3-dihydro-[1,4]dioxino[2,3-b]pyridin-6-yl)ethyl)-5-ethyl-2-methylpiperazin-1-yl)-3,9-dimethyl-2-oxo-3,9-dihydro-2H-purin-8-yl)acetonitrile (A159);
2-(6-((2S,5R)-4-(1-(7-chloro-3,3-dimethyl-2,3-dihydro-[1,4]dioxino[2,3-b]pyridin-6-yl)ethyl)-2,5-dimethylpiperazin-1-yl)-9-ethyl-3-methyl-2-oxo-3,9-dihydro-2H-purin-8-yl)acetonitrile (A160);
2-(9-ethyl-6-((2S,5R)-4-(1-(7-fluoro-3,3-dimethyl-2,3-dihydro-[1,4]dioxino[2,3-b]pyridin-6-yl)ethyl)-2,5-dimethylpiperazin-1-yl)-3-methyl-2-oxo-3,9-dihydro-2H-purin-8-yl)acetonitrile (A161);
2-(6-((2S,5R)-4-(1-(7-fluoro-3,3-dimethyl-2,3-dihydro-[1,4]dioxino[2,3-b]pyridin-6-yl)ethyl)-2,5-dimethylpiperazin-1-yl)-3,9-dimethyl-2-oxo-3,9-dihydro-2H-purin-8-yl)acetonitrile (A162);
2-(9-ethyl-6-((2S,5R)-4-(1-(8-fluoro-3,3-dimethyl-2,3-dihydro-[1,4]dioxino[2,3-b]pyridin-6-yl)ethyl)-2,5-dimethylpiperazin-1-yl)-3-methyl-2-oxo-3,9-dihydro-2H-purin-8-yl)acetonitrile (A163);
2-(6-((2S,5R)-4-(1-(8-fluoro-3,3-dimethyl-2,3-dihydro-[1,4]dioxino[2,3-b]pyridin-6-yl)ethyl)-2,5-dimethylpiperazin-1-yl)-3,9-dimethyl-2-oxo-3,9-dihydro-2H-purin-8-yl)acetonitrile (A164);
2-(9-(2,2-difluoroethyl)-6-((2S,5R)-4-(1-(3,3-dimethyl-2,3-dihydro-[1,4]dioxino[2,3-b]pyridin-6-yl)ethyl)-2,5-dimethylpiperazin-1-yl)-3-methyl-2-oxo-3,9-dihydro-2H-purin-8-yl)acetonitrile (A165);
2-(9-(2,2-difluoroethyl)-6-((2S,5R)-4-(1-(2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile (A166);
2-(6-((2S,5R)-4-(1-(2,2-difluorobenzo[d][1,3]dioxol-5-yl)ethyl)-2,5-dimethylpiperazin-1-yl)-3,9-dimethyl-2-oxo-3,9-dihydro-2H-purin-8-yl)acetonitrile (A167);
2-(9-ethyl-6-((2S,5R)-4-(1-(2-ethyl-2-methylbenzo[d][1,3]dioxol-5-yl)ethyl)-2,5-dimethylpiperazin-1-yl)-3-methyl-2-oxo-3,9-dihydro-2H-purin-8-yl)acetonitrile (A168);
2-(6-((2S,5R)-2,5-dimethyl-4-(1-(spiro[benzo[d][1,3]dioxole-2,1′-cyclobutan]-5-yl)ethyl)piperazin-1-yl)-9-ethyl-3-methyl-2-oxo-3,9-dihydro-2H-purin-8-yl)acetonitrile (A169);
2-(6-((2S,5R)-4-(1-(6-chloro-2,2-dimethylbenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile (A170);
2-(6-((2S,5R)-4-(1-(7-chloro-2,2-dimethylbenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile (A171);
2-(6-((2S,5R)-4-1-(4-chloro-2,2-dimethylbenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile (A172);
2-(9-ethyl-6-((2S,5R)-4-(1-(7-fluoro-2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile (A173);
2-(9-ethyl-6-((2S,5R)-4-(1-(6-fluoro-2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile (A174);
2-(9-ethyl-6-((2S,5R)-4-(1-(4-fluoro-2,2-dimethylbenzo[d][1,3]dioxol-5-yl)ethyl)-2,5-dimethylpiperazin-1-yl)-3-methyl-2-oxo-3,9-dihydro-2H-purin-8-yl)acetonitrile (A175);
2-(6-((2S,5R)-4-(1-(4,6-difluoro-2,2-dimethylbenzo[d][1,3]dioxol-5-yl)ethyl)-2,5-dimethylpiperazin-1-yl)-9-ethyl-3-methyl-2-oxo-3,9-dihydro-2H-purin-8-yl)acetonitrile (A176);
2-(6-((2S,5R)-2,5-dimethyl-4-(1-(2,2,7-trimethylbenzo[d][1,3]dioxol-5-yl)ethyl)piperazin-1-yl)-9-ethyl-3-methyl-2-oxo-3,9-dihydro-2H-purin-8-yl)acetonitrile (A177);
2-(9-ethyl-6-((2S,5R)-4-(1-(2-isopropyl-1,3-dioxoisoindolin-5-yl)ethyl)-2,5-dimethylpiperazin-1-yl)-3-methyl-2-oxo-3,9-dihydro-2H-purin-8-yl)acetonitrile (A178);
2-(6-((2S,5R)-4-(1-(2,2-dimethyl-3-oxo-2,3-dihydrobenzofuran-6-yl)ethyl)-2,5-dimethylpiperazin-1-yl)-9-ethyl-3-methyl-2-oxo-3,9-dihydro-2H-purin-8-yl)acetonitrile (A179);
2-(6-((2S,5R)-4-(1-(2,2-dimethyl-2,3-dihydrofuro[2,3-b]pyridin-6-yl)ethyl)-2,5-dimethylpiperazin-1-yl)-9-ethyl-3-methyl-2-oxo-3,9-dihydro-2H-purin-8-yl)acetonitrile (A180);
2-(6-((2S,5R)-4-(1-(3,3-dimethyl-2,3-dihydrofuro[3,2-b]pyridin-5-yl)ethyl)-2,5-dimethylpiperazin-1-yl)-9-ethyl-3-methyl-2-oxo-3,9-dihydro-2H-purin-8-yl)acetonitrile (A181);
2-(6-((2S,5R)-4-(3,3-dimethyl-2,3-dihydrofuro[3,2-b]pyridine-5-carbonyl)-2,5-dimethylpiperazin-1-yl)-9-ethyl-3-methyl-2-oxo-3,9-dihydro-2H-purin-8-yl)acetonitrile (A182);
2-(6-((2S,5R)-4-(1-(3,3-dimethyl-2,3-dihydrobenzofuran-5-yl)ethyl)-2,5-dimethylpiperazin-1-yl)-9-ethyl-3-methyl-2-oxo-3,9-dihydro-2H-purin-8-yl)acetonitrile (A183);
2-(6-((2S,5R)-4-(1-(3,3-dimethyl-2,3-dihydro-1H-inden-5-yl)ethyl)-2,5-dimethylpiperazin-1-yl)-9-ethyl-3-methyl-2-oxo-3,9-dihydro-2H-purin-8-yl)acetonitrile (A184);
2-(9-ethyl-6-((2S,5R)-4-(1-(7-fluoro-2,2-dimethylbenzo[d][1,3]dioxol-4-yl)ethyl)-2,5-dimethylpiperazin-1-yl)-3-methyl-2-oxo-3,9-dihydro-2H-purin-8-yl)acetonitrile (A185);
2-(6-((2S,5R)-4-(1-(2,2-dimethylbenzo[d][1,3]dioxol-4-yl)ethyl)-2,5-dimethylpiperazin-1-yl)-9-ethyl-3-methyl-2-oxo-3,9-dihydro-2H-purin-8-yl)acetonitrile (A186);
2-(6-((2S,5R)-4-(1-(2,2-difluorobenzo[d][1,3]dioxol-4-yl)ethyl)-2,5-dimethylpiperazin-1-yl)-9-ethyl-3-methyl-2-oxo-3,9-dihydro-2H-purin-8-yl)acetonitrile (A187);
2-(3-ethyl-6-fluoro-7-((2S,5R)-4-(1-(4-fluoro-2,2-dimethylbenzo[d][1,3]dioxol-5-yl)ethyl)-2,5-dimethylpiperazin-1-yl)-4-methyl-5-oxo-4,5-dihydro-3H-imidazo[4,5-b]pyridin-2-yl)acetonitrile (A190);
2-(7-((2S,5R)-4-(1-(2,2-dimethylbenzo[d][1,3]dioxol-5-yl)ethyl)-2,5-dimethylpiperazin-1-yl)-3-ethyl-6-fluoro-4-methyl-5-oxo-4,5-dihydro-3H-imidazo[4,5-b]pyridin-2-yl)acetonitrile (A188);
2-(3-ethyl-6-fluoro-7-((2S,5R)-4-(1-(7-fluoro-2,2-dimethylbenzo[d][1,3]dioxol-5-yl)ethyl)-2,5-dimethylpiperazin-1-yl)-4-methyl-5-oxo-4,5-dihydro-3H-imidazo[4,5-b]pyridin-2-yl)acetonitrile (A189);
2-(3-ethyl-6-fluoro-7-((2S,5R)-4-(1-(4-fluoro-2,2-dimethylbenzo[d][1,3]dioxol-5-yl)ethyl)-2,5-dimethylpiperazin-1-yl)-4-methyl-5-oxo-4,5-dihydro-3H-imidazo[4,5-b]pyridin-2-yl)acetonitrile (A190);
2-(6-((2S,5R)-2,5-dimethyl-4-(1-(2-methyl-4-oxo-4H-chromen-7-yl)ethyl)piperazin-1-yl)-9-ethyl-3-methyl-2-oxo-3,9-dihydro-2H-purin-8-yl)acetonitrile (A191);
2-(6-((2S,5R)-2,5-dimethyl-4-(1-(3-methyl-4-oxo-3,4-dihydro-2H-benzo[e][1,3]oxazin-6-yl)ethyl)piperazin-1-yl)-9-ethyl-3-methyl-2-oxo-3,9-dihydro-2H-purin-8-yl)acetonitrile (A192);
2-(6-((2S,5R)-4-(1-(1,3-dimethyl-4-oxo-1,4-dihydroquinolin-6-yl)ethyl)-2,5-dimethylpiperazin-1-yl)-9-ethyl-3-methyl-2-oxo-3,4,5,9-tetrahydro-2H-purin-8-yl)acetonitrile (A193);
2-(7-((2S,5R)-4-(1-(4-fluoro-2-(trifluoromethyl)phenyl)ethyl)-2,5-dimethylpiperazin-1-yl)-3,4-dimethyl-5-oxo-4,5-dihydro-3H-imidazo[4,5-b]pyridin-2-yl)acetonitrile (A2);
2-(7-((2S,5R)-4-(1-(4-fluorophenyl)ethyl)-2,5-dimethylpiperazin-1-yl)-3,4-dimethyl-5-oxo-4,5-dihydro-3H-imidazo[4,5-b]pyridin-2-yl)acetonitrile (A3);
2-(7-((2S,5R)-2,5-dimethyl-4-(1-(4-methyl-2-(trifluoromethyl)phenyl)ethyl)piperazin-1-yl)-3,4-dimethyl-5-oxo-4,5-dihydro-3H-imidazo[4,5-b]pyridin-2-yl)acetonitrile (A8);
2-(7-((2S,5R)-2,5-dimethyl-4-(1-(4-methyl-2-(trifluoromethyl)phenyl)ethyl)piperazin-1-yl)-6-fluoro-3,4-dimethyl-5-oxo-4,5-dihydro-3H-imidazo[4,5-b]pyridin-2-yl)acetonitrile (A15);
2-(7-((2S,5R)-2,5-dimethyl-4-(1-(3-methyl-2-(trifluoromethyl)phenyl)ethyl)piperazin-1-yl)-6-fluoro-3,4-dimethyl-5-oxo-4,5-dihydro-3H-imidazo[4,5-b]pyridin-2-yl)acetonitrile (A17);
2-(6-((2S,5R)-2,5-dimethyl-4-(1-(4-methyl-2-(trifluoromethyl)phenyl)ethyl)piperazin-1-yl)-3,9-dimethyl-2-oxo-3,9-dihydro-2H-purin-8-yl)acetonitrile (A32);
2-(6-((2S,5R)-4-(1-(3-(difluoromethyl)-5-fluorophenyl)ethyl)-2,5-dimethylpiperazin-1-yl)-9-ethyl-3-methyl-2-oxo-3,9-dihydro-2H-purin-8-yl)acetonitrile (A38);
2-(6-((2S,5R)-4-(1-(3-(1,1-difluoroethyl)-4-methoxyphenyl)ethyl)-2,5-dimethylpiperazin-1-yl)-9-ethyl-3-methyl-2-oxo-3,9-dihydro-2H-purin-8-yl)acetonitrile (A39);
2-(6-((2S,5R)-2,5-dimethyl-4-(1-(4-(trifluoromethyl)phenyl)ethyl)piperazin-1-yl)-9-ethyl-3-methyl-2-oxo-3,9-dihydro-2H-purin-8-yl)acetonitrile (A40);
2-(6-((2S,5R)-2,5-dimethyl-4-(1-(m-tolyl)ethyl)piperazin-1-yl)-9-ethyl-3-methyl-2-oxo-3,9-dihydro-2H-purin-8-yl)acetonitrile (A45);
2-(9-ethyl-6-((2S,5R)-4-(1-(3-methoxyphenyl)ethyl)-2,5-dimethylpiperazin-1-yl)-3-methyl-2-oxo-3,9-dihydro-2H-purin-8-yl)acetonitrile (A46);
2-(9-ethyl-6-((2S,5R)-4-(1-(2-methoxypyridin-4-yl)ethyl)-2,5-dimethylpiperazin-1-yl)-3-methyl-2-oxo-3,9-dihydro-2H-purin-8-yl)acetonitrile (A47);
2-(6-((2S,5R)-4-(1-(7-chloro-3,3-dimethyl-2,3-dihydrobenzo[b][1,4]dioxin-6-yl)ethyl)-2,5-dimethylpiperazin-1-yl)-9-ethyl-3-methyl-2-oxo-3,9-dihydro-2H-purin-8-yl)acetonitrile (A73);
2-(6-((2S,5R)-4-(1-(2,2-dimethyl-2,3-dihydro-[1,4]dioxino[2,3-c]pyridin-7-yl)ethyl)-2,5-dimethylpiperazin-1-yl)-9-ethyl-3-methyl-2-oxo-3,9-dihydro-2H-purin-8-yl)acetonitrile (A75);
2-(6-((2S,5R)-4-(1-(2,2-dimethyl-2,3-dihydro-[1,4]dioxino[2,3-b]pyridin-7-yl)ethyl)-2,5-dimethylpiperazin-1-yl)-9-ethyl-3-methyl-2-oxo-3,9-dihydro-2H-purin-8-yl)acetonitrile (A76);
2-(6-((2S,5R)-2,5-dimethyl-4-(1-(1-oxo-2-(2,2,2-trifluoroethyl)isoindolin-5-yl)ethyl)piperazin-1-yl)-9-ethyl-3-methyl-2-oxo-3,9-dihydro-2H-purin-8-yl)acetonitrile (A82);
2-(6-((2S,5R)-2,5-dimethyl-4-(1-(2,3,3-trimethyl-1-oxoisoindolin-5-yl)ethyl)piperazin-1-yl)-9-ethyl-3-methyl-2-oxo-3,9-dihydro-2H-purin-8-yl)acetonitrile (A84);
2-(6-((2S,5R)-2,5-dimethyl-4-(1-(3-methyl-2,3-dihydro-[1,4]dioxino[2,3-b]pyridin-6-yl)ethyl)piperazin-1-yl)-9-ethyl-3-methyl-2-oxo-3,9-dihydro-2H-purin-8-yl)acetonitrile (A89);
2-(6-((2S,5R)-2,5-dimethyl-4-(1-(2-methylquinolin-7-yl)ethyl)piperazin-1-yl)-9-ethyl-3-methyl-2-oxo-3,9-dihydro-2H-purin-8-yl)acetonitrile (A90);
2-(6-((2S,5R)-2,5-dimethyl-4-(1-(2-methyl-1-oxo-1,2-dihydroisoquinolin-7-yl)ethyl)piperazin-1-yl)-9-ethyl-3-methyl-2-oxo-3,9-dihydro-2H-purin-8-yl)acetonitrile (A92);
2-(6-((2S,5R)-2,5-dimethyl-4-(1-(2-methyl-1-oxo-1,2,3,4-tetrahydroisoquinolin-7-yl)ethyl)piperazin-1-yl)-9-ethyl-3-methyl-2-oxo-3,9-dihydro-2H-purin-8-yl)acetonitrile (A93);
2-(6-((2S,5R)-2,5-dimethyl-4-(1-(2-methyl-3-oxoisoindolin-5-yl)ethyl)piperazin-1-yl)-9-ethyl-3-methyl-2-oxo-3,9-dihydro-2H-purin-8-yl)acetonitrile (A100);
2-(6-((2S,5R)-2,5-dimethyl-4-(1-(2-methyl-1-oxoisoindolin-5-yl)ethyl)piperazin-1-yl)-9-ethyl-3-methyl-2-oxo-3,9-dihydro-2H-purin-8-yl)acetonitrile (A101);
2-(6-((2S,5R)-2,5-dimethyl-4-(1-(2-(trifluoromethyl)thiazol-4-yl)ethyl)piperazin-1-yl)-9-ethyl-3-methyl-2-oxo-3,9-dihydro-2H-purin-8-yl)acetonitrile (A104);
2-(6-((2S,5R)-2,5-dimethyl-4-(1-(2-(trifluoromethyl)thiazol-5-yl)ethyl)piperazin-1-yl)-9-ethyl-3-methyl-2-oxo-3,9-dihydro-2H-purin-8-yl)acetonitrile (A105);
2-(6-((2S,5R)-2,5-dimethyl-4-(1-(1-methyl-6-oxo-1,6-dihydropyridin-3-yl)ethyl)piperazin-1-yl)-9-ethyl-3-methyl-2-oxo-3,9-dihydro-2H-purin-8-yl)acetonitrile (A106);
2-(6-((2S,5R)-2,5-dimethyl-4-(1-(1-methyl-2-oxo-1,2-dihydropyridin-4-yl)ethyl)piperazin-1-yl)-9-ethyl-3-methyl-2-oxo-3,9-dihydro-2H-purin-8-yl)acetonitrile (A107);
2-(6-((2S,5R)-4-(1-(3-(difluoromethyl)phenyl)ethyl)-2,5-dimethylpiperazin-1-yl)-9-ethyl-3-methyl-2-oxo-3,9-dihydro-2H-purin-8-yl)acetonitrile (A109);
2-(7-((2S,5R)-2,5-dimethyl-4-(1-(quinoxalin-6-yl)ethyl)piperazin-1-yl)-4-methyl-5-oxo-4,5-dihydrothiazolo[5,4-b]pyridin-2-yl)acetonitrile (A194);
2-(7-((2S,5R)-4-(1-(4-fluoro-2-(trifluoromethyl)phenyl)ethyl)-2,5-dimethylpiperazin-1-yl)-4-methyl-5-oxo-4,5-dihydrothiazolo[5,4-b]pyridin-2-yl)acetonitrile (A195); or
2-(7-((2S,5R)-2,5-dimethyl-4-(1-(4-methyl-2-(trifluoromethyl)phenyl) ethyl)piperazin-1-yl)-4-methyl-5-oxo-4,5-dihydrothiazolo[5,4-b]pyridin-2-yl)acetonitrile (A196).

31. A pharmaceutical composition comprising one or more compounds of any one of claims 1-30, or a stereoisomer or pharmaceutically acceptable salt thereof, and a pharmaceutically acceptable excipient.

32. A method of treating a disease, comprising administering to a patient in need of such treatment a therapeutically effective amount of a compound of any one of claims 1-30, wherein the disease is cancer.
